(19) United States
(12) Patent Application Publication
(10) Pub. No.: US 2024/0173267 A1
(43) Pub. Date: May 30, 2024

(54) COMPOUNDS, COMPOSITIONS, AND METHODS OF USING THEREOF

(71) Applicant: Oncorus, Inc., Cambridge, MA (US)

(72) Inventors: Jessica Deterling, Cambridge, MA (US); Sean Essex, Cambridge, MA (US); Lorena Lerner, Cambridge, MA (US); Qi-Ying Hu, Needham, MA (US); Christophe Quéva, Cambridge, MA (US)

(73) Assignee: Oncorus, Inc., Cambridge, MA (US)

(21) Appl. No.: 18/276,527

(22) PCT Filed: Jan. 6, 2022
(86) PCT No.: PCT/US2022/011463

§ 371 Date: Aug. 9, 2023

Related U.S. Application Data
(60) Provisional application No. 63/181,899, filed on Apr. 29, 2021.
(60) Provisional application No. 63/181,917, filed on Apr. 29, 2021.
(60) Provisional application No. 63/147,959, filed on Feb. 10, 2021.

(51) Int. Cl. A61K 9/51; A61K 35/768; A61K 48/00; A61P 35/00; C07C 333/04; C07D 205/04; C07D 207/09; C07D 211/54; C07D 223/06; C07D 295/15; C12N 15/86

(52) U.S. Cl. CPC A61K 9/5123; A61K 9/5146; A61K 35/768; A61K 48/0025; A61P 35/00; C07C 333/04; C07D 205/04; C07D 207/09; C07D 211/54; C07D 223/06; C07D 295/15; C12N 15/86; C12N 2770/32033; C12N 2770/32043; C12N 2770/32071

(57) ABSTRACT

The present disclosure includes, among other things, lipids, compositions, and methods useful for delivering a polynucleotide or oligonucleotide, e.g., viral genome.

### CROSS-REFERENCE TO RELATED APPLICATIONS

This application claims priority to and the benefit of U.S. Provisional Patent Application No. 63/147,959, filed on Feb. 10, 2021; U.S. Provisional Patent Application No. 63/181,899, filed on Apr. 29, 2021; and U.S. Provisional Patent Application No. 63/181,917, filed on Apr. 29, 2021, the contents of each of which are incorporated herein by reference in their entireties.

### BACKGROUND

Lipids are used as materials for nucleic acid (NA) delivery owing to their ability to form lipid-NA nanoparticles that encapsulate nucleic acid-based therapeutics, e.g., siRNA or mRNA, for delivery to target cells upon parenteral administration (Zimmermann, 2006, Nature, doi: 10.1038/nature04688; August at al., 2021, Nat Med, doi: 10.1038/s41591-021-01573-6).

The delivery of nucleic acids for treating and immunizing subjects has been a goal for several years. Various approaches have been tested, including the use of DNA or RNA, e.g., DNA or RNA of viral or non-viral delivery vehicles (or even no delivery vehicle, in a “naked” vaccine), of replicating or non-replicating vectors, or of viral or non-viral vectors.

There remains a need for further and improved nucleic acid-based treatments and vaccines and, in particular, for improved ways of delivering nucleic acid therapeutics.

### SUMMARY

The present application provides lipids, compositions, and methods useful for delivering a polynucleotide or oligonucleotide.

Accordingly, in one aspect, provided herein are compounds of Formula (I):

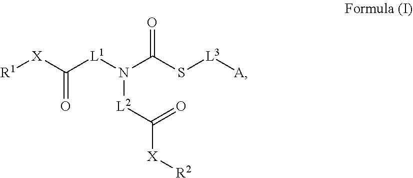

or a pharmaceutically acceptable salt or solvate thereof, wherein:

A is —N(CH₂R^{N1})(CH₂R^{N2}) or a 4-7-membered heterocyclyl ring containing at least one N, wherein the 4-7-membered heterocyclyl ring is optionally substituted with 0-6 R³;
each X is independently —O—, —N(R¹)—, or —N(R²)—
R¹ is selected from the group consisting of optionally substituted C₁-C₃₁ aliphatic and steroidyl;
R² is selected from the group consisting of optionally substituted C₁-C₃₁ aliphatic and steroidyl;
R³ is optionally substituted C₁-C₆ aliphatic;
R^{N1} and R^{N2} are each independently hydrogen, hydroxy-C₁-C₆ alkyl, C₂-C₆ alkenyl, or a C₃-C₇ cycloalkyl;
L¹ is selected from the group consisting of an optionally substituted C₁-C₂₀ alkylene chain and a bivalent optionally substituted C₂-C₂₀ alkenylene chain;
L² is selected from the group consisting of an optionally substituted C₁-C₂₀ alkylene chain and a bivalent optionally substituted C₂-C₂ alkenylene chain; and
L³ is a bond, an optionally substituted C₁-C₆ alkylene chain, or a bivalent optionally substituted C₃-C₇ cycloalkylene; and
with the proviso that when A is —N(CH₃)(CH₃) and X is O, L³ is not an C₁-C₆ alkylene chain.

In some embodiments, the compound is a compound of Formula (I-a):

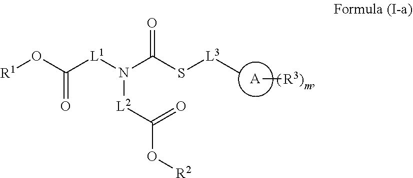

or a pharmaceutically acceptable salt or solvate thereof, wherein: m is 0, 1, 2, 3, 4, 5, or 6.

In some embodiments, A contains one or more S. In some embodiments, A is an optionally substituted 4-7-membered heterocyclyl ring containing exactly one N. In some embodiments, A is an optionally substituted 5-6-membered heterocyclyl ring. In some embodiments, A is an optionally substituted 6 membered heterocyclyl ring containing exactly one N. In some embodiments, A is a tertiary amine.

In some embodiments, the compound is a compound of Formula (I-b):

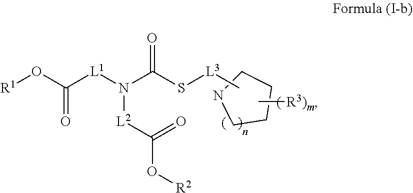

or a pharmaceutically acceptable salt or solvate thereof, wherein: n is 0, 1, 2, or 3; and m is 0, 1, 2, 3, 4, 5, or 6.

In some embodiments, the compound is a compound of Formula (I-bii):

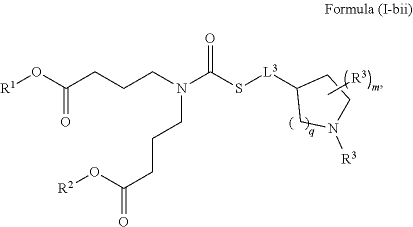

or a pharmaceutically acceptable salt or solvate thereof, wherein: m is 0, 1, 2, or 3; and p and q are each independently 0, 1, 2, or 3, wherein q+p is less than or equal to 3.

In some embodiments, n is 1. In some embodiments, n is 2. In some embodiments, n is 3. In some embodiments, m is 0. In some embodiments, m is 1.

In some embodiments, the compound is a compound of Formula (I-c):

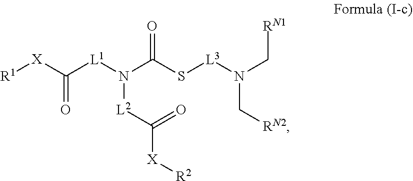

or a pharmaceutically acceptable salt or solvate thereof.

In some embodiments, X is O. In some embodiments, X is NR¹ or NR².

In some embodiments, R¹ and R² are each independently optionally substituted C₁-C₃₁ alkyl or optionally substituted C₂-C₃₁ alkenyl. In some embodiments, R¹ and R² are the same. In some embodiments, R¹ and R² are each independently optionally substituted C₁₀-C₂₀ alkyl. In some embodiments, R¹ and R² are each independently branched C₁₀-C₂₀ alkyl. In some embodiments, R¹ and R² are the different. In some embodiments, R¹ is optionally substituted C₆-C₂₀ alkenyl and R² is optionally substituted C₁₀-C₂₀ alkyl. In some embodiments, R¹ is C₆-C₂₀ alkenyl and R² is branched C₁₀-C₂₀ alkyl.

In some embodiments, L¹ is an optionally substituted C₁-C₁₀ alkylene chain and L² is an optionally substituted C₁-C₁₀ alkylene chain. In some embodiments, L¹ is an optionally substituted C₁-C₅ alkylene chain and L² is an optionally substituted C₁-C₅ alkylene chain. In some embodiments, L¹ is an optionally substituted C₁-C₃ alkylene chain and L² is an optionally substituted C₁-C₃ alkylene chain. In some embodiments, L¹ and L² are each —CH₂CH₂CH₂—.

In some embodiments, L³ is a C₁-C₃ alkylene chain. In some embodiments, L³ is a bond. In some embodiments, L³ is a bivalent C₃-C₇ cycloalkylene. In some embodiments, L³ is a bond. In some embodiments, L³ is —CH₂—.

In some embodiments, the number of carbon atoms between the S of the thiolate and the closest N comprised in A is 2-10. In some embodiments, the number of carbon atoms between the S of the thiolate and the closest N comprised in A is 2-8. In some embodiments, the number of carbon atoms between the S of the thiolate and the closest N comprised in A is 2-5. In some embodiments, the number of carbon atoms between the S of the thiolate and the closest N comprised in A is 2-4. In some embodiments, the number of carbon atoms between the S of the thiolate and the closest N comprised in A is 3.

In some embodiments, R³ is C₁-C₆ alkyl or C₁-C₆ alkenyl, wherein each C₁-C₆ alkyl or C₁-C₆ alkenyl is optionally substitute with 1-3 C₃-C₆ cycloalkyl or —OH. In some embodiments, R³ is C₁-C₃ alkyl. In some embodiments, R³ is —CH₃.

In some embodiments, RNi and R^{N2} are each independently selected from hydrogen, hydroxy-C₁-C₃ alkyl, C₂-C₄ alkenyl, or C₃-C₄ cycloalkyl. In some embodiments, RNi and R^{N2} are each independently selected from hydrogen, —CH₂CH═CH₂, —CH₂CH₂OH, or

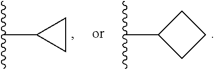

In some embodiments, R^{N1} and R^{N2} are the same. In some embodiments, RNi and R^{N2} are different. In some embodiments, one of RNi and R^{N2} is hydrogen and the other one is

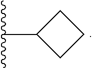 [SMILES: *C1CCC1]

In another aspect, provided herein are compounds selected from the group consisting of

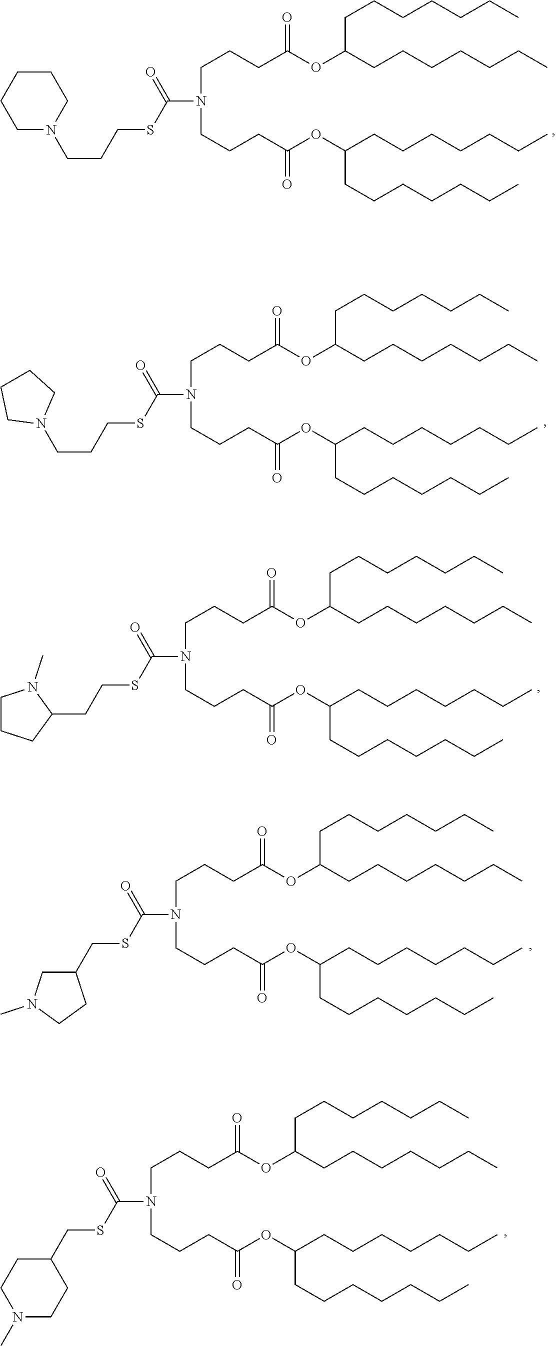

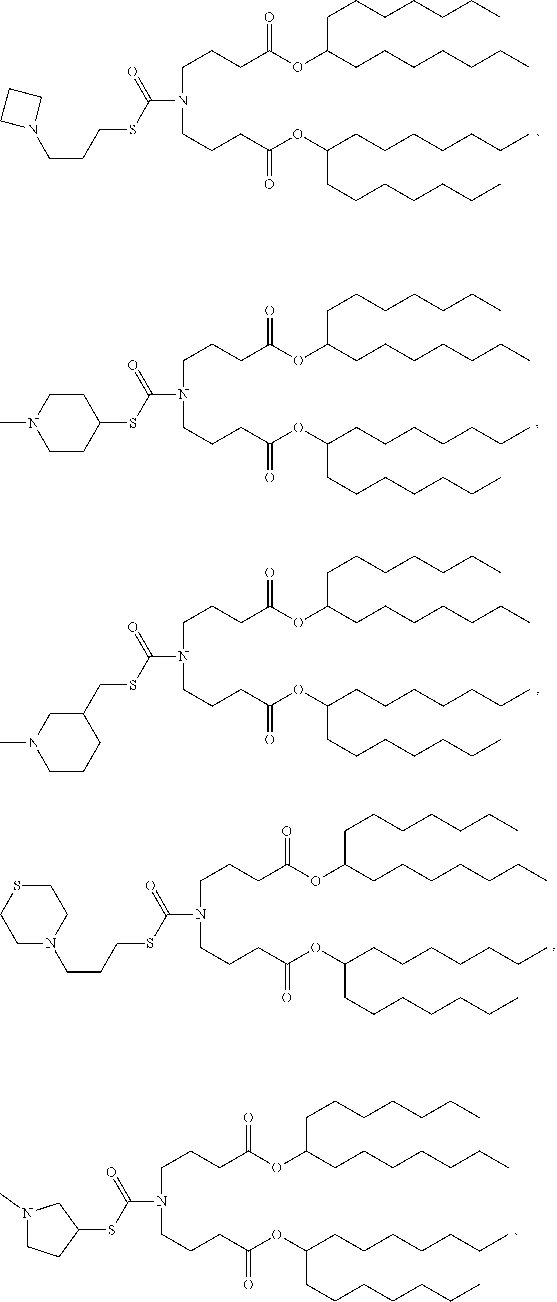

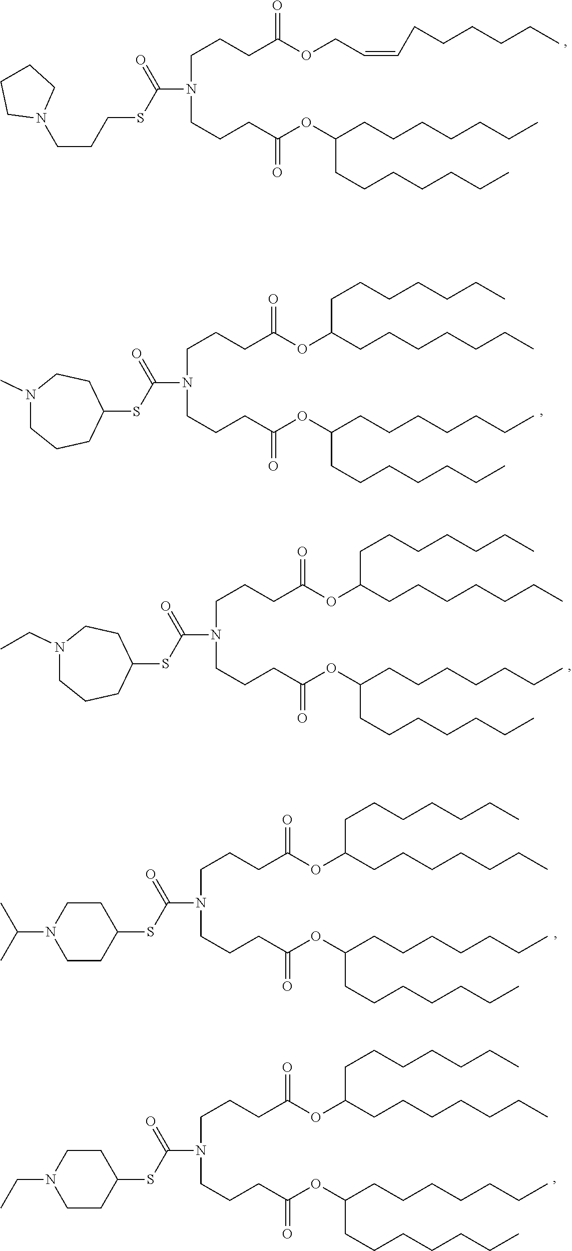

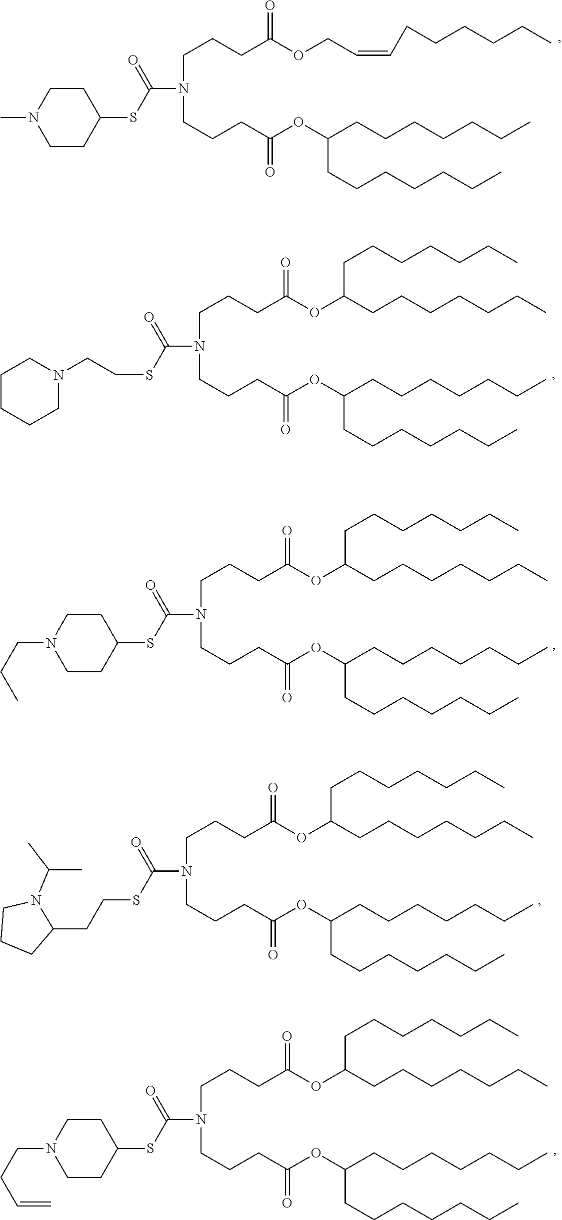 [SMILES: CCCCCC/C=C\COC(=O)CCCN(CCCC(=O)OC(CCCCCCC)CCCCCCC)C(=O)SC1CCN(C)CC1 ; CCCCCCCC(CCCCCCC)OC(=O)CCCN(CCCC(=O)OC(CCCCCCC)CCCCCCC)C(=O)SCCN1CCCCC1 ; CCCCCCCC(CCCCCCC)OC(=O)CCCN(CCCC(=O)OC(CCCCCCC)CCCCCCC)C(=O)SC1CCN(CCC)CC1 ; CCCCCCCC(CCCCCCC)OC(=O)CCCN(CCCC(=O)OC(CCCCCCC)CCCCCCC)C(=O)SCCC1CCCN1C(C)C ; C=CCCN1CCC(SC(=O)N(CCCC(=O)OC(CCCCCCC)CCCCCCC)CCCC(=O)OC(CCCCCCC)CCCCCCC)CC1]

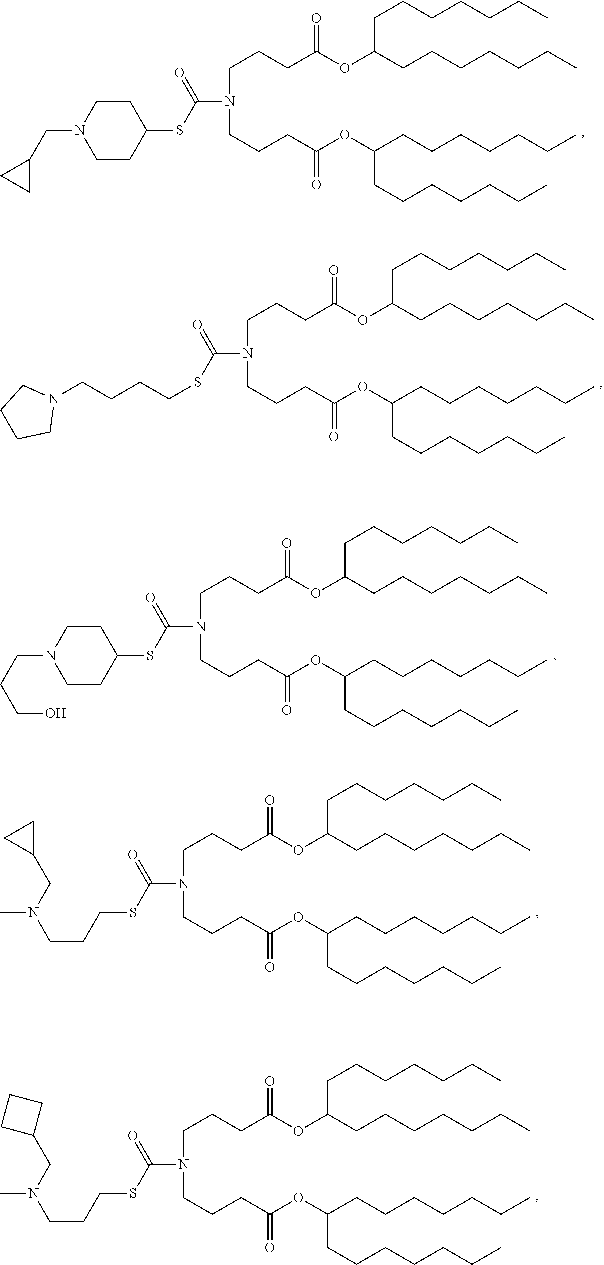

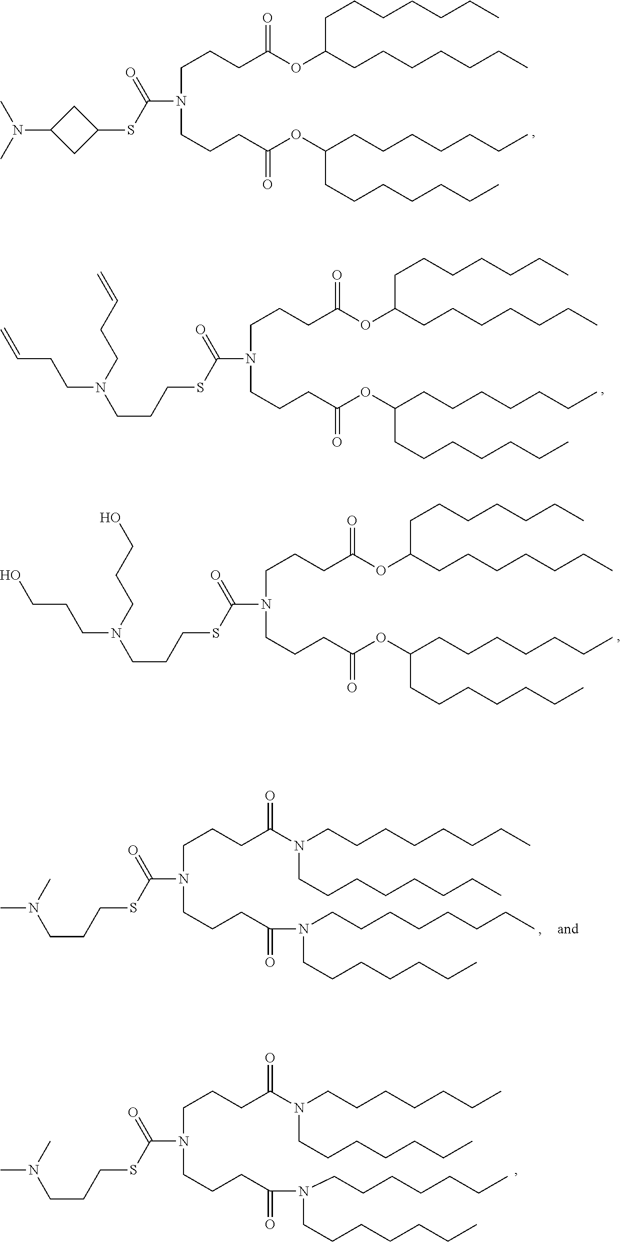

or a pharmaceutically acceptable salt or solvate thereof.

In some embodiments, the compound is

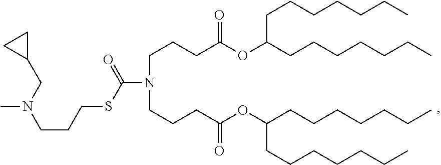 [SMILES: CCCCCCCC(CCCCCCC)OC(=O)CCCN(CCCC(=O)OC(CCCCCCC)CCCCCCC)C(=O)SCCCN(C)CC1CC1]

or a pharmaceutically acceptable salt or solvate thereof.

In some embodiments, the compound is

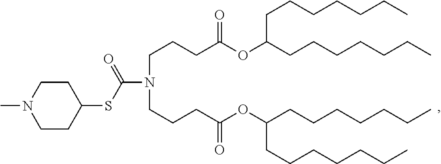 [SMILES: CCCCCCCC(CCCCCCC)OC(=O)CCCN(CCCC(=O)OC(CCCCCCC)CCCCCCC)C(=O)SC1CCN(C)CC1]

or a pharmaceutically acceptable salt or solvate thereof.

In some embodiments, the compound is

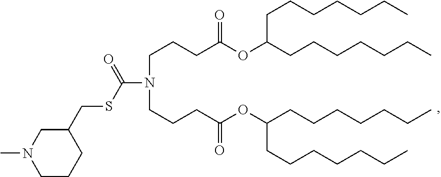 [SMILES: CCCCCCCC(CCCCCCC)OC(=O)CCCN(CCCC(=O)OC(CCCCCCC)CCCCCCC)C(=O)SCC1CCCN(C)C1]

or a pharmaceutically acceptable salt or solvate thereof.

In another aspect, provided herein are compounds selected from

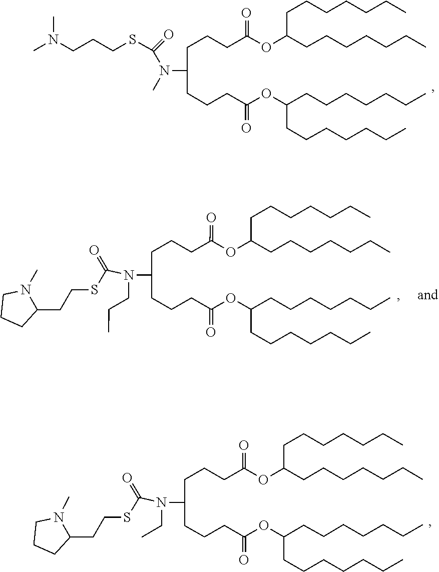 [SMILES: CCCCCCCC(CCCCCCC)OC(=O)CCCC(CCCC(=O)OC(CCCCCCC)CCCCCCC)N(C)C(=O)SCCCN(C)C ; CCCCCCCC(CCCCCCC)OC(=O)CCCC(CCCC(=O)OC(CCCCCCC)CCCCCCC)N(CCC)C(=O)SCCC1CCCN1C ; CCCCCCCC(CCCCCCC)OC(=O)CCCC(CCCC(=O)OC(CCCCCCC)CCCCCCC)N(CC)C(=O)SCCC1CCCN1C]

or a pharmaceutically acceptable salt or solvate thereof.

In another aspect, provided herein are compounds of Formula (A)

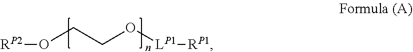

or a pharmaceutically acceptable salt thereof, wherein:
n is an integer between 10 to 200, inclusive of all endpoints;
L^{P1} is —[(CH₂)₀₋₃—C(O)O]₁₋₃—, —(CH₂)₀₋₃—C(O)O—(CH₂)₁₋₃—OC(O)—, or —C(O)N(H)—;
R^{P1} is C₅-C₂₅ alkyl or C₅-C₂₅ alkenyl; and
R^{P2} is hydrogen or —CH₃,
with the proviso that Formula (A) is not HO—(CH₂CH₂O)ₙ—C(O)N(H)—(CH₂)₁₇CH₃.

In some embodiments, L^{P1} is —CH₂C(O)O—, —CH₂CH₂C(O)O—, —CH₂C(O)OCH₂C(O)O—, —CH₂C(O)OCH₂CH₂OC(O)—, or —C(O)N(H)—. In some embodiments, the compound is a compound of Formula (A-a), Formula (A-b), Formula (A-c), Formula (A-d), or Formula (A-e):

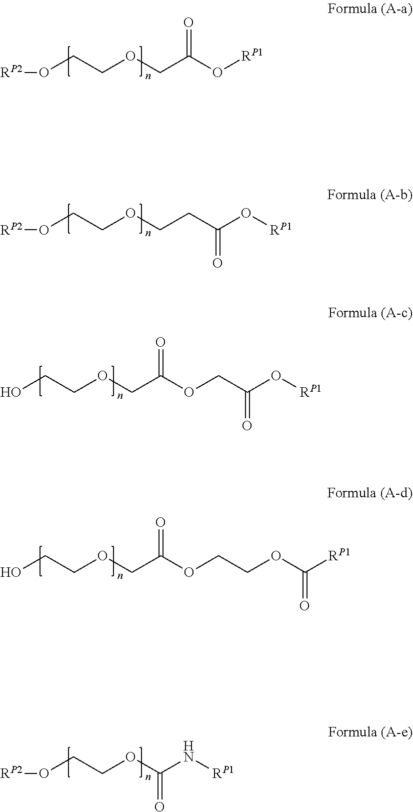

or a pharmaceutically acceptable salt thereof.

In some embodiments, R¹ is C₁₄-C₁₈ alkyl or C₁₄-Cis alkenyl. In some embodiments, R¹ is C₁₄ alkyl, C₁₆ alkyl, or Cis alkyl.

In some embodiments, n is on average about 20, about 40, about 45, about 50, about 68, about 75, or about 100.

In some embodiments, the compound selected from the group consisting of:
HO—(CH₂CH₂O)ₙ—CH₂C(O)O—(CH₂)₁₇CH₃, n is on average about 45;
H₃CO—(CH₂CH₂O)ₙ—CH₂C(O)O—(CH₂)₁₇CH₃, n is on average about 45;
HO—(CH₂CH₂O)ₙ—CH₂C(O)O—(CH₂)₁₅CH₃, n is on average about 45;
HO—(CH₂CH₂O)ₙ—CH₂C(O)O—(CH₂)₁₃CH₃, n is on average about 45; and
HO—(CH₂CH₂O)ₙ—C(O)N(H)—(CH₂)₁₇CH₃, n is on average about 45;
or a pharmaceutically acceptable salt thereof.

Also provided herein are lipid nanoparticle (LNP) comprising a compound disclosed herein, e.g., a compound of Formula (I). In some embodiments, the LNP comprises a helper lipid, a structural lipid, and a polyethyleneglycol (PEG)-lipid, such as a PEG-lipid disclosed herein. In some embodiments, the PEG-lipid is a compound of Formula (A′):

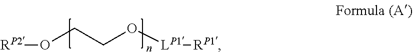

or a pharmaceutically acceptable salt thereof, wherein:
n is an integer between 10 to 200, inclusive of all endpoints;
L^{P1′} is a bond, —C(O)—, —[(CH₂)₀₋₃—C(O)O]₁₋₃—, —(CH₂)₀₋₃—C(O)O—(CH₂)₁₋₃—OC(O)—, or —C(O)N(H)—;
R^{P1′} is C₅-C₂₅ alkyl or C₅-C₂₅ alkenyl; and
R^{P2′} is hydrogen or —CH₃.

In some embodiments, the PEG-lipid is a compound selected from the group consisting of:
HO—(CH₂CH₂₀)ₙ—CH₂C(O)O—(CH₂)₁₇CH₃, n is on average about 45;
H₃CO—(CH₂CH₂₀)ₙ—CH₂C(O)O—(CH₂)₁₇CH₃, n is on average about 45;
HO—(CH₂CH₂₀)ₙ—CH₂C(O)O—(CH₂)₁₅CH₃, n is on average about 45;
HO—(CH₂CH₂₀)ₙ—CH₂C(O)O—(CH₂)₁₃CH₃, n is on average about 45; and
HO—(CH₂CH₂₀)ₙ—C(O)N(H)—(CH₂)₁₇CH₃, n is on average about 45;
or a pharmaceutically acceptable salt thereof.

In some embodiments, the PEG-lipid is a compound selected from the group consisting of:
HO—(CH₂CH₂₀)ₙ—(CH₂)₁₇CH₃, n is on average about 100;
HO—(CH₂CH₂₀)ₙ—(CH₂)₁₇CH₃, n is on average about 20;
HO—(CH₂CH₂₀)ₙ—(CH₂)₁₅CH₃, n is on average about 20; and
HO—(CH₂CH₂₀)ₙ—C₁₈H₃₅, n is on average about 20;
or a pharmaceutically acceptable salt thereof.

In some embodiments, the PEG-lipid is a compound selected from the group consisting of:
HO—(CH₂CH₂₀)ₙ—C(O)—(CH₂)₁₄CH₃, n is on average about 100;
HO—(CH₂CH₂₀)ₙ—C(O)—(CH₂)₁₄CH₃, n is on average about 50;
HO—(CH₂CH₂₀)ₙ—C(O)—(CH₂)₁₄CH₃, n is on average about 40;
HO—(CH₂CH₂₀)ₙ—C(O)—(CH₂)₁₆CH₃, n is on average about 100;
HO—(CH₂CH₂₀)ₙ—C(O)—(CH₂)₁₆CH₃, n is on average about 50; and
HO—(CH₂CH₂₀)ₙ—C(O)—(CH₂)₁₆CH₃, n is on average about 40;
   or a pharmaceutically acceptable salt thereof.

In some embodiments, the PEG-lipid is DMG-PEG(2000) or DPG-PEG(2000).

In another aspect, provided herein are LNPs comprising a polyethyleneglycol (PEG)-lipid, an ionizable lipid, a helper lipid, and a structural lipid, wherein the LNP has a molar ratio of about 0.001% to about 5% PEG-lipid, and wherein the PEG-lipid is a compound of Formula (A″):

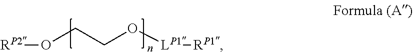

or a pharmaceutically acceptable salt thereof, wherein:
n is an integer between 10 to 200, inclusive of all endpoints;
L^{P1″} is a bond, —[(CH₂)₀₋₃—C(O)O]₁₋₃—, —(CH₂)₀₋₃—C(O)O—(CH₂)₁₋₃—OC(O)—, or —C(O)N(H)—;
R^{P1″} is C₅-C₂₅ alkyl or C₅-C₂₅ alkenyl; and
R^{P2″} is hydrogen or —CH₃.

In some embodiments, L^{P1″} is a bond, —CH₂C(O)O—, —CH₂CH₂C(O)O—, —CH₂C(O)OCH₂C(O)O—, —CH₂C(O)OCH₂CH₂₀C(O)—, or —C(O)N(H)—. In some embodiments, the PEG-lipid is a compound of Formula (A″-a), Formula (A″-b), Formula (A″-c), Formula (A″-cd), Formula (A″-e), or Formula (A″-f):

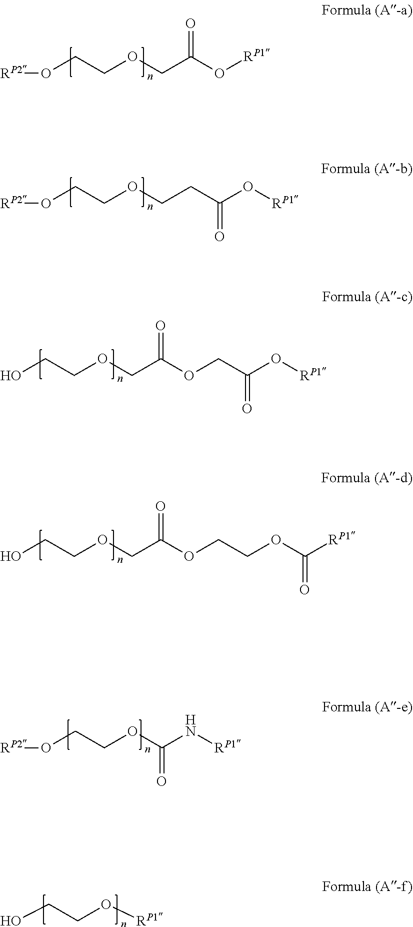

or a pharmaceutically acceptable salt thereof.

In some embodiments, R^{P1″} is C₁₄-C₁₈ alkyl or C₁₄-C₁₈ alkenyl. In some embodiments, R^{P1″} is C₁₄ alkyl, C₁₆ alkyl, or Cis alkyl.

In some embodiments, the PEG-lipid is a compound of Formula (A″-f1), Formula (A″-f2), or Formula (A″-f3):

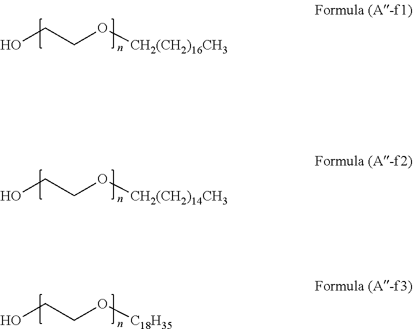

or a pharmaceutically acceptable salt thereof.

In another aspect, provided herein are LNPs comprising a polyethyleneglycol (PEG)-lipid, an ionizable lipid, a helper lipid, a structural lipid, and a nucleic acid molecule encoding a viral genome, wherein the LNP has a molar ratio of about 0.001% to about 5% PEG-lipid, and wherein the PEG-lipid is a compound of Formula (B):

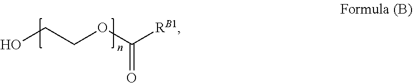

or a pharmaceutically acceptable salt thereof, wherein: n is an integer between 10 to 200, inclusive of all endpoints; and R^{B1} IS C₅-C₂₅ alkyl or C₅-C₂₅ alkenyl. In some embodiments, R^{B1} is C₁₅-C₁₇ alkyl or C₁₅-C₁₇ alkenyl. In some embodiments, the PEG-lipid is a compound of Formula (B-a) or Formula (B-b):

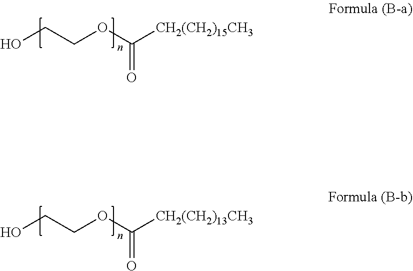

or a pharmaceutically acceptable salt thereof.

In some embodiments, n is on average about 20, about 40, about 45, about 50, about 68, about 75, or about 100. In some embodiments, the PEG-lipid comprises a PEG moiety having an average molecular weight of about 200 daltons to about 10,000 daltons, about 500 daltons to about 7,000 daltons, about 800 daltons to about 6,000 daltons, about 1,000 daltons to about 5,000 daltons, or about 1,500 to about 3,500 daltons. In some embodiments, the PEG-lipid comprises a PEG moiety having an average molecular weight of about 800, about 900, about 1,000, about 1,500, about 1,750, about 2,000, about 2,250, about 2,500, about 2,750, about 3,000, about 3,250, about 3,500, about 3,750, about 4,000, about 4,500, or about 5,000 daltons. In some embodiments, he PEG-lipid comprises a PEG moiety having an average molecular weight of about 800, about 900, about 1,000 daltons, about 1,500, about 2,000, about 2,500, about 3,000, about 3,500, about 4,000, about 4,500, or about 5,000 daltons.

In some embodiments, the PEG-lipid is selected from the group consisting of: HO—(CH₂CH₂₀)ₙ—(CH₂)₁₇CH₃, n is on average about 100; HO—(CH₂CH₂₀)ₙ—(CH₂)₁₇CH₃, n is on average about 20; HO—(CH₂CH₂₀)ₙ—(CH₂)₁₅CH₃, n is on average about 20; and HO—(CH₂CH₂₀)ₙ—C₁₈H₃₅, n is on average about 20.

In some embodiments, the PEG-lipid is a compound selected from the group consisting of: HO—(CH₂CH₂₀)ₙ—CH₂C(O)O—(CH₂)₁₇CH₃, n is on average about 45; H₃CO—(CH₂CH₂₀)ₙ—CH₂C(O)O—(CH₂)₁₇CH₃, n is on average about 45; HO—(CH₂CH₂₀)ₙ—CH₂C(O)O—(CH₂)₁₅CH₃, n is on average about 45; HO—(CH₂CH₂₀)ₙ—CH₂C(O)O—(CH₂)₁₃CH₃, n is on average about 45; and HO—(CH₂CH₂₀)ₙ—C(O)N(H)—(CH₂)₁₇CH₃, n is on average about 45.

In some embodiments, the PEG-lipid is selected from the group consisting of: HO—(CH₂CH₂₀)ₙ—C(O)—(CH₂)₁₄CH₃, n is on average about 100; HO—(CH₂CH₂₀)ₙ—C(O)—(CH₂)₁₄CH₃, n is on average about 50; HO—(CH₂CH₂₀)ₙ—C(O)—(CH₂)₁₄CH₃, n is on average about 40; HO—(CH₂CH₂₀)ₙ—C(O)—(CH₂)₁₆CH₃, n is on average about 100; HO—(CH₂CH₂₀)ₙ—C(O)—(CH₂)₁₆CH₃, n is on average about 50; and HO—(CH₂CH₂₀)ₙ—C(O)—(CH₂)₁₆CH₃, n is on average about 40.

In some embodiments, the ionizable lipid is selected from DLinDMA, DLin-KC2-DMA, DLin-MC3-DMA (MC3), COATSOME® SS-LC (former name: SS-18/4PE-13), COATSOME® SS-EC (former name: SS-33/4PE-15), COATSOME® SS—OC, COATSOME® SS—OP, Di((Z)-non-2-en-1-yl)₉-((4-dimethylamino)butanoyl)oxy)heptadecanedioate (L-319), N-(2,3-dioleoyloxy)propyl)-N,N,N-trimethylammonium chloride (DOTAP), or a mixture thereof.

In some embodiments, the ionizable lipid is a compound of Formula (II-1):

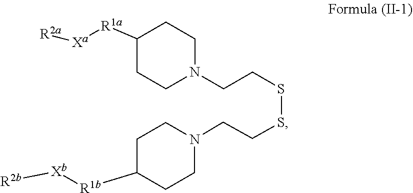

or a pharmaceutically acceptable salt or solvate thereof, wherein:
R^{1a} and R^{1b} are each independently C₁-C₈ aliphatic or —O(C₁-C₈ aliphatic)-, wherein the O atom, when present, is bonded to the piperidine ring;
X^{a} and X^{b} are each independently —C(O)O—*, —OC(O)—*, —C(O)N(Rₓ¹)—*, —N(Rₓ¹)C(O)—*, —O(C═O)N(Rₓ¹)—*, —N(Rₓ¹)(C═O)O—*, or —O—, wherein -* indicates the attachment point to R^{2a} or R^{2b}, respectively, and wherein each occurrence of Rₓ¹ is independently selected from hydrogen and optionally substituted C₁-C₄ alkyl; and
R^{2a} and R^{2b} are each independently a sterol residue, a liposoluble vitamin residue, or an C₁₃-C₂₃ aliphatic.

In some embodiments, the ionizable lipid is a compound of Formula (II-2):

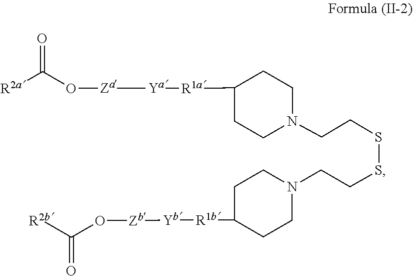

or a pharmaceutically acceptable salt or solvate thereof, wherein: R^{1a′} and R^{1b′} are each independently C₁-C₈ alkylene or —O(C₁-C₈ alkylene), wherein the O atom, when present, is bonded to the piperidine ring; Y^{a′} and Y^{b′} are each independently —C(O)O—*, —OC(O)—*, —C(O)N(Rₓ¹)—*, —N(Rₓ¹)C(O)—*, —O(C═O)N(Rₓ¹)—*, —N(Rₓ¹)(C═O)O—*, —N(Rₓ¹)C(O)N(Rₓ¹)—, or —O—, wherein -* indicates the attachment point to R^{2a} or R^{2b}, and wherein each occurrence of Rₓ¹ is independently selected from hydrogen and optionally substituted C₁-C₄ alkyl; Z^{a′} and Z^{b′} are each independently optionally substituted arylene-C₀-C₈ alkylene or optionally substituted arylene-C₀-C₈ heteroalkylene, wherein the alkylene or heteroalkylene group is bonded to Y^{a′} and Y^{b′}, respectively; R^{2a′} and R^{2b′} are each independently a sterol residue, a liposoluble vitamin residue, or an C₁₂-C₂₂ aliphatic.

In some embodiments, the ionizable lipid is a compound of Formula (II-1a):

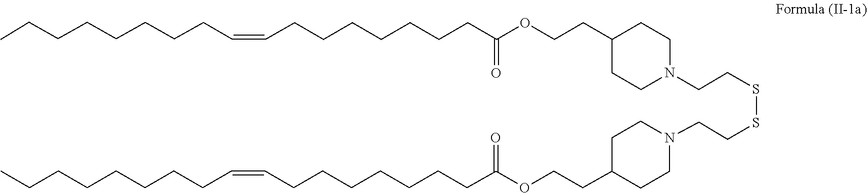 [SMILES: CCCCCCCC/C=C\CCCCCCCC(=O)OCCC1CCN(CCSSCCN2CCC(CCOC(=O)CCCCCCC/C=C\CCCCCCCC)CC2)CC1]

In some embodiments, the ionizable lipid is a compound of Formula (II-2a):

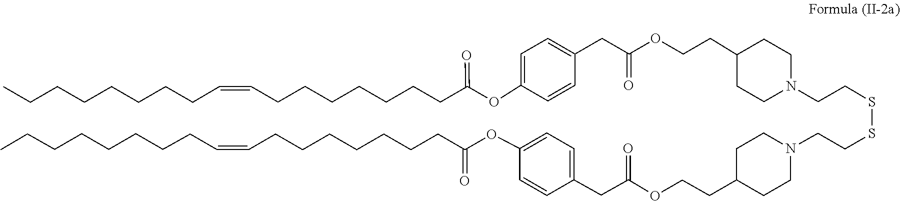 [SMILES: CCCCCCCC/C=C\CCCCCCCC(=O)Oc1ccc(CC(=O)OCCC2CCN(CCSSCCN3CCC(CCOC(=O)Cc4ccc(OC(=O)CCCCCCC/C=C\CCCCCCCC)cc4)CC3)CC2)cc1]

In some embodiments, the ionizable lipid is a compound disclosed herein, e.g., a compound of Formula (I).

In some embodiments, the helper lipid is selected from distearoyl-sn-glycero-phosphoethanolamine, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoylphosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), monomethyl-phosphatidylethanolamine, dimethylphosphatidylethanolamine, 18-1-trans PE,1-stearoyl-2-oleoylphosphatidyethanolamine (SOPE), hydrogenated soy phosphatidylcholine (HSPC), egg phosphatidylcholine (EPC), dioleoylphosphatidylserine (DOPS), sphingomyelin (SM), dimyristoyl phosphatidylcholine (DMPC), dimyristoyl phosphatidylglycerol (DMPG), distearoylphosphatidylglycerol (DSPG), dierucoylphosphatidylcholine (DEPC), palmitoyloleyolphosphatidylglycerol (POPG), dielaidoyl-phosphatidylethanolamine (DEPE), lecithin, phosphatidylethanolamine, lysolecithin, lysophosphatidylethanolamine, phosphatidyl serine, phosphatidylinositol, sphingomyelin, egg sphingomyelin (ESM), cephalin, cardiolipin, phosphatidicacid, cerebrosides, dicetylphosphate, lysophosphatidylcholine, dilinoleoylphosphatidylcholine, or a mixture thereof. In some embodiments, the helper lipid is DSPC.

In some embodiments, the structural lipid is a steroid. In some embodiments, the structural lipid is cholesterol.

In some embodiments, the LNP induces a reduced immune response in vivo as compared to a control LNP lacking a PEG-lipid of Formula (A″) or an ionizable lipid disclosed herein (e.g., an ionizable lipid of Formula (I)). In some embodiments, the immune response is accelerated blood clearance (ABC) of the LNP. In some embodiments, the immune response is an IgM response.

In some embodiments, a LNP provided herein comprises a compound of Formula (I), a structural lipid that is cholesterol, a helper lipid that is DSPC, and a PEG-lipid that is a compound of Formula (A″). In some embodiments, the compound of Formula (I) is selected from the group consisting of:

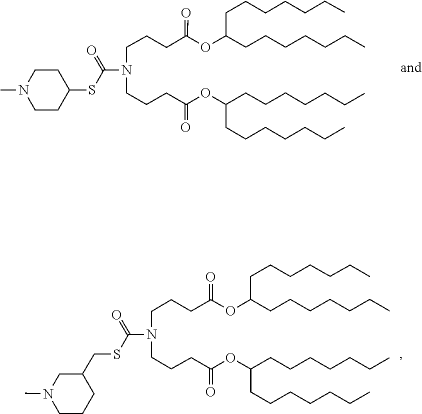

or a pharmaceutically acceptable salt thereof. In some embodiments, the PEG-lipid is a compound of selected from the group consisting of: HO—(CH₂CH₂₀)ₙ—(CH₂)₁₇CH₃, n is on average about 100; HO—(CH₂CH₂₀)ₙ—CH₂C(O)O—(CH₂)₁₃CH₃, n is on average about 45; and HO—(CH₂CH₂₀)ₙ—CH₂C(O)O—(CH₂)₁₇CH₃, n is on average about 45.

In some embodiments, a LNP provided herein comprises a compound of Formula (II-1a), a structural lipid that is cholesterol, a helper lipid that is DSPC, and a PEG-lipid that is a compound of Formula (A″). In some embodiments, the PEG-lipid is selected from the group consisting of: HO—(CH₂CH₂₀)ₙ—CH₂C(O)O—(CH₂)₁₇CH₃, n is on average about 45; H₃CO—(CH₂CH₂₀)ₙ—CH₂C(O)O—(CH₂)₁₇CH₃, n is on average about 45; HO—(CH₂CH₂₀)ₙ—CH₂C(O)O—(CH₂)₁₅CH₃, n is on average about 45; HO—(CH₂CH₂₀)ₙ—CH₂C(O)O—(CH₂)₁₃CH₃, n is on average about 45; and HO—(CH₂CH₂₀)ₙ—C(O)N(H)—(CH₂)₁₇CH₃, n is on average about 45. In some embodiments, the PEG-lipid is HO—(CH₂CH₂₀)ₙ—(CH₂)₁₇CH₃, n is on average about 100.

In some embodiments, a LNP provided herein comprises a compound of Formula (II-1a), a structural lipid that is cholesterol, a helper lipid that is DSPC, and a PEG-lipid that is a compound of Formula (B). In some embodiments, the PEG-lipid is selected from the group consisting of: HO—(CH₂CH₂₀)ₙ—C(O)—(CH₂)₁₆CH₃, n is on average about 100; HO—(CH₂CH₂₀)ₙ—C(O)—(CH₂)₁₆CH₃, n is on average about 50; and HO—(CH₂CH₂₀)ₙ—C(O)—(CH₂)₁₆CH₃, n is on average about 40.

In some embodiments, a LNP provided herein comprises a molar ratio of about 40% to about 70%. such as about 45% to about 55%, or about 49% to about 64% of an ionizable lipid disclosed herein, e.g., a compound of Formula (I). In some embodiments, the LNP comprises a molar ratio of about 40%, about 45%, about 50% about 55%, about 58%, or about 60% of an ionizable lipid disclosed herein, e.g., a compound of Formula (1).

In some embodiments, a LNP provided herein comprises a molar ratio of about 0.1% to about 4%, such as about 0.2% to about 0.8 mol %, about 0.4% to about 0.6 mol %, about 0.7% to about 1.3%, about 1.2% to about 1.8%, or about 1% to about 3.5 mol % PEG-lipid. In some embodiments, the LNP comprises a molar ratio of about 0.25%, about 0.5%, about 1.5%, or about 3% PEG-lipid.

In some embodiments, a LNP provided herein comprises a molar ratio of about 5% to about 50%, such as about 5% to about 10%, about 25% to about 35%, or about 35% to about 50% structural lipid. In some embodiments, the LNP comprises a molar ratio of about 20%, about 22.5%, about 25%, about 27.5%, about 30%, about 3²0.5%, about 35%, about 37.5%, about 40%, about 42.5%, about 45%, or about 50% structural lipid.

In some embodiments, a LNP provided herein comprises a molar ratio of about 5% to about 50%, such as about 5% to about 10%, about 10% to about 25%, or about 25% to about 50% helper lipid. In some embodiments, the LNP comprises a molar ratio of about 5%, about 7%, about 9%, about 12%, about 15%, about 20%, about 25%, or about 30% helper lipid.

In some embodiments, a LNP provided herein comprises a molar ratio of about 45% to about 55% of ionizable lipid, about 5% to about 9% helper lipid, about 36% to about 44% structural lipid, and about 2.5% to about 3.5% PEG-lipid.

In some embodiments, a LNP provided herein comprises a molar ratio of about 45% to about 55% of an ionizable lipid disclosed herein, e.g., a compound of Formula (I), about 5% to about 9% DSPC, about 36% to about 44% cholesterol, and about 2.5% to about 3.5% DMG-PEG(2000).

In some embodiments, a LNP provided herein comprises a molar ratio of about 49% to about 60% of an ionizable lipid disclosed herein, e.g., a compound of Formula (I), about 18% to about 22% helper lipid, about 22% to about 28% structural lipid, and about 0.2% to about 0.8% PEG-lipid, e.g., selected from the group consisting of: HO—(CH₂CH₂₀)ₙ—CH₂C(O)O—(CH₂)₁₇CH₃, n is on average about 45; H₃CO—(CH₂CH₂₀)ₙ—CH₂C(O)O—(CH₂)₁₇CH₃, n is on average about 45; HO—(CH₂CH₂₀)ₙ—CH₂C(O)O—(CH₂)₁₅CH₃, n is on average about 45; HO—(CH₂CH₂₀)ₙ—CH₂C(O)O—(CH₂)₁₃CH₃, n is on average about 45; and HO—(CH₂CH₂₀)ₙ—C(O)N(H)—(CH₂)₁₇CH₃, n is on average about 45.

In some embodiments, a LNP provided herein comprises a molar ratio of about 44% to about 54% of an ionizable lipid disclosed herein, e.g., a compound of Formula (II-1a), about 19% to about 25% helper lipid, about 25% to about 33% structural lipid, and about 0.2% to about 0.8% PEG-lipid, e.g., selected from the group consisting of: HO—(CH₂CH₂₀)ₙ—(CH₂)₁₇CH₃, n is on average about 100; HO—(CH₂CH₂₀)ₙ—(CH₂)₁₇CH₃, n is on average about 20; HO—(CH₂CH₂₀)ₙ—(CH₂)₁₅CH₃, n is on average about 20; HO—(CH₂CH₂₀)ₙ—C₁₈H₃₅, n is on average about 20; HO—(CH₂CH₂₀)ₙ—C(O)—(CH₂)₁₄CH₃, n is on average about 100; HO—(CH₂CH₂₀)ₙ—C(O)—(CH₂)₁₄CH₃, n is on average about 50; HO—(CH₂CH₂₀)ₙ—C(O)—(CH₂)₁₄CH₃, n is on average about 40; HO—(CH₂CH₂₀)ₙ—C(O)—(CH₂)₁₆CH₃, n is on average about 100; HO—(CH₂CH₂₀)ₙ—C(O)—(CH₂)₁₆CH₃, n is on average about 50; and HO—(CH₂CH₂₀)ₙ—C(O)—(CH₂)₁₆CH₃, n is on average about 40.

In some embodiments, a LNP provided herein comprises a molar ratio of about 44% to about 54% of an ionizable lipid disclosed herein, e.g., a compound of Formula (II-1a), about 19% to about 25% helper lipid, about 24% to about 32% structural lipid, and about 1.2% to about 1.8% PEG-lipid, e.g., selected from the group consisting of: HO—(CH₂CH₂₀)ₙ—(CH₂)₁₇CH₃, n is on average about 100; HO—(CH₂CH₂₀)ₙ—(CH₂)₁₇CH₃, n is on average about 20; HO—(CH₂CH₂₀)ₙ—(CH₂)₁₅CH₃, n is on average about 20; HO—(CH₂CH₂₀)ₙ—C₁₈H₃₅, n is on average about 20; HO—(CH₂CH₂₀)ₙ—C(O)—(CH₂)₁₄CH₃, n is on average about 100; HO—(CH₂CH₂₀)ₙ—C(O)—(CH₂)₁₄CH₃, n is on average about 50; HO—(CH₂CH₂₀)ₙ—C(O)—(CH₂)₁₄CH₃, n is on average about 40; HO—(CH₂CH₂₀)ₙ—C(O)—(CH₂)₁₆CH₃, n is on average about 100; HO—(CH₂CH₂₀)ₙ—C(O)—(CH₂)₁₆CH₃, n is on average about 50; and HO—(CH₂CH₂₀)ₙ—C(O)—(CH₂)₁₆CH₃, n is on average about 40.

In some embodiments, a LNP provided herein comprises a molar ratio of about 44% to about 54% ionizable lipid of an ionizable lipid disclosed herein, e.g., a compound of Formula (II-1a), about 8% to about 14% helper lipid, about 35% to about 43% structural lipid, and about 1.2% to about 1.8% PEG-lipid, e.g., selected from the group consisting of: HO—(CH₂CH₂₀)ₙ—(CH₂)₁₇CH₃, n is on average about 100; HO—(CH₂CH₂₀)ₙ—(CH₂)₁₇CH₃, n is on average about 20; HO—(CH₂CH₂₀)ₙ—(CH₂)₁₅CH₃, n is on average about 20; HO—(CH₂CH₂₀)ₙ—C₁₈H₃₅, n is on average about 20; HO—(CH₂CH₂₀)ₙ—C(O)—(CH₂)₁₄CH₃, n is on average about 100; HO—(CH₂CH₂₀)ₙ—C(O)—(CH₂)₁₄CH₃, n is on average about 50; HO—(CH₂CH₂₀)ₙ—C(O)—(CH₂)₁₄CH₃, n is on average about 40; HO—(CH₂CH₂₀)ₙ—C(O)—(CH₂)₁₆CH₃, n is on average about 100; HO—(CH₂CH₂₀)ₙ—C(O)—(CH₂)₁₆CH₃, n is on average about 50; and HO—(CH₂CH₂₀)ₙ—C(O)—(CH₂)₁₆CH₃, n is on average about 40.

In some embodiments, the LNP encapsulates a payload molecule. In some embodiments, the payload molecule comprises one or more of nucleic acids, anionic proteins, anionic peptides, or a combination thereof. In some embodiments, the payload molecule comprises a nucleic acid molecule. In some embodiments, the nucleic acid molecule comprises single-stranded RNA (ssRNA), an siRNA, a microRNA, an mRNA, a circular RNA, a small activating RNA, a guide RNA for CRISPR, a self-amplifying RNA, a viral RNA (vRNA), a single-stranded DNA (ssDNA), a double-stranded DNA (dsDNA), a complementary DNA (cDNA), a closed circular DNA (ccDNA), a replicon, or a combination thereof. In some embodiments, the nucleic acid molecule comprises a nucleotide sequence encoding one or more therapeutic proteins. In some embodiments, the therapeutic protein is a cytokine (e.g., erythropoietin), a coagulation factor, an antibody, a bispecific T cell engager, or a combination thereof. In some embodiments, the nucleic acid molecule comprises a nucleotide sequence derived from a viral genome. In some embodiments, the viral genome is a positive single-stranded RNA viral genome a positive single-stranded RNA viral genome. In some embodiments, the viral genome encodes an oncolytic virus (e.g., Coxsackievirus A21 (CVA21), Seneca Valley virus (SVV), Togaviridae, or Alphavirus (e.g., Sindbis virus, Semliki Forest virus, Ross River virus, or Chikungunya virus)). In some embodiments, the payload molecule comprises a synthetic RNA viral genome encoding a coxsackievirus, and optionally wherein the coxsackievirus is a CVA21 strain. In some embodiments, the payload molecule comprises a synthetic RNA viral genome encoding an SVV. In some embodiments, the payload molecule further encodes an exogenous protein, wherein the exogenous protein is a fluorescent protein, an enzymatic protein, a cytokine, a chemokine, an antigen-binding molecule capable of binding to a cell surface receptor, or a ligand for a cell-surface receptor. In some embodiments, the viral genome is a positive single-stranded RNA viral genome. In some embodiments, the viral genome encodes an oncolytic virus (e.g., Coxsackievirus A21 (CVA21) or Seneca Valley virus (SVV), Togaviridae, or Alphavirus (e.g., Sindbis virus, Semliki Forest virus, Ross River virus, or Chikungunya virus)). In some embodiments, the viral genome is a synthetic RNA viral genome encoding a coxsackievirus, and optionally wherein the coxsackievirus is a CVA21 strain. In some embodiments, the viral genome is a synthetic RNA viral genome encoding an SVV. In some embodiments, the viral genome further comprises an exogenous protein, wherein the exogenous protein is a fluorescent protein, an enzymatic protein, a cytokine, a chemokine, an antigen-binding molecule capable of binding to a cell surface receptor, or a ligand for a cell-surface receptor.

In some embodiments, the LNP has a lipid-nitrogen-to-phosphate (N:P) ratio of about 1 to about 25. In some embodiments, the LNP has a N:P ratio of about 14. In some embodiments, the LNP has a N:P ratio of about 9.

Also provided herein are pharmaceutical compositions comprising a compound disclosed herein or a LNP disclosed herein and pharmaceutically acceptable excipient, carrier or diluent.

Also provided herein are pharmaceutical compositions comprising: (1) a payload molecule; and (2) a LNP disclosed herein. In some embodiments, the payload molecule comprises a nucleic acid molecule. In some embodiments, the payload molecule comprises a synthetic RNA viral genome encoding a Coxsackievirus or an SVV. In some embodiments, the viral genome comprised in the LNP is a synthetic RNA viral genome encoding a Coxsackievirus or an SVV. In some embodiments, the pharmaceutical composition further comprises a pharmaceutically acceptable carrier.

In some embodiments, the pharmaceutical composition of the disclosure has a half-life in vivo comparable to that of a pre-determined threshold value. In some embodiments, the pharmaceutical composition of the disclosure has a half-life in vivo greater than that of a pre-determined threshold value. In some embodiments, the pharmaceutical composition of the disclosure has a half-life in vivo shorter than that of a pre-determined threshold value. In some embodiments, the pharmaceutical composition has an AUC in vivo comparable to that of a pre-determined threshold value. In some embodiments, the pharmaceutical composition has an AUC in vivo greater than that of a pre-determined threshold value. In some embodiments, the pharmaceutical composition has an AUC in vivo less than that of a pre-determined threshold value. In some embodiments, the pre-determined threshold value is determined in a control composition comprising the same payload molecule and LNP except that the LNP lacks a PEG-lipid of Formula (A′) or an ionizable lipid disclosed herein (e.g., an ionizable lipid of Formula (I)).

In some embodiments, the LNP has an average diameter of about 50 nm, about 60 nm, about 70 nm, about 80 nm, about 90 nm, about 100 nm, about 110 nm, about 120 nm, or about 125 nm. In some embodiments, the encapsulation efficiency of the payload molecule by the LNP is about 70%, about 75%, about 80%, about 85%, about 90%, about 91%, about 92%, about 93%, about 94%, about 95%, about 96%, about 97%, about 98%, about 99%, or 100%. In some embodiments, the pharmaceutical composition has a total lipid concentration of about 10 mM, about 20 mM, about 30 mM, about 40 mM, or about 50 mM. In some embodiments, the pharmaceutical composition is formulated at a pH of about 2.5, about 3, about 3.5, about 4, about 4.5, about 5, about 5.5, or about 6.

In some embodiments, the pharmaceutical composition is formulated for multiple administrations. In some embodiments, a subsequent administration is administered at least 3 days, at least 5 days, at least 7 days, at least 9 days, at least 11 days, at least 14 days, or at least 21 days after a first administration.

Also provided herein are methods of treating a disease or disorder, comprising administering to a patient in need thereof a LNP disclosed herein or a pharmaceutical composition disclosed herein.

In some embodiments, the disease or disorder is cancer. In some embodiments, the cancer is selected is selected from the group consisting of lung cancer, breast cancer, ovarian cancer, cervical cancer, prostate cancer, testicular cancer, colorectal cancer, colon cancer, pancreatic cancer, liver cancer, renal cell carcinoma, gastric cancer, head and neck cancer, thyroid cancer, malignant glioma, glioblastoma, melanoma, B-cell chronic lymphocytic leukemia, multiple myeloma, monoclonal gammopathy of undetermined significance (MGUS), Merkel cell carcinoma, diffuse large B-cell lymphoma (DLBCL), sarcoma, a neuroblastoma, a neuroendocrine cancer, a rhabdomyosarcoma, a medulloblastoma, a bladder cancer, and marginal zone lymphoma (MZL). In some embodiments, the cancer is selected from the groups consisting of lung cancer, breast cancer, colon cancer, pancreatic cancer, bladder cancer, renal cell carcinoma, ovarian cancer, gastric cancer, and liver cancer. In some embodiments, the cancer is renal cell carcinoma, lung cancer, or liver cancer. In some embodiments, the lung cancer is small cell lung cancer or non-small cell lung cancer (e.g., squamous cell lung cancer or lung adenocarcinoma). In some embodiments, the liver cancer is hepatocellular carcinoma (HCC) (e.g., Hepatitis B virus associated HCC). In some embodiments, the prostate cancer is treatment-emergent neuroendocrine prostate cancer. In some embodiments, the cancer is lung cancer, liver cancer, prostate cancer (e.g., CRPC-NE), bladder cancer, pancreatic cancer, colon cancer, gastric cancer, breast cancer, neuroblastoma, renal cell carcinoma, ovarian cancer, rhabdomyosarcoma, medulloblastoma, neuroendocrine cancer, Merkel cell carcinoma, or melanoma. In some embodiments, the cancer is small cell lung cancer (SCLC) or neuroblastoma.

In some embodiments, the administration of the pharmaceutical composition delivers a payload into tumor cells. In some embodiments, the administration of the pharmaceutical composition inhibits the tumor growth.

In some embodiments, the LNP or pharmaceutical composition is administered parenterally. In some embodiments, the LNP or pharmaceutical composition is administered is administered intratumorally and/or intravenously.

### BRIEF DESCRIPTION OF THE DRAWINGS

FIG. 1A is a graph depicting the results of a dynamic light scattering experiment of LNP compositions spiked with different cryo-protectants. FIG. 1B is a graph depicting the encapsulation efficiency of these LNP compositions measured by RiboGreen.

FIG. 2A is a graph depicting the results of a dynamic light scattering experiment of LNP compositions post-concentration or post-dialysis. FIG. 2B is a graph depicting the encapsulation efficiency of these LNP compositions measured by RiboGreen.

FIG. 3A is a graph depicting the results of a PK study in mice of LNP compositions comprising PEG2k-DPG as PEG-lipid. FIG. 3B is a graph depicting the results of a PK study in mice of LNP compositions comprising Brij S100 as PEG-lipid.

FIG. 4A is a graph depicting the results of a dynamic light scattering experiment of LNP compositions comprising PEG-lipid of the disclosure. FIG. 4B is a graph depicting the encapsulation efficiency of these LNP compositions measured by RiboGreen.

FIG. 5A is a graph depicting the results of a H446 mouse tumor model showing the growth of tumor upon repeat dose of the LNP compositions of the disclosure. FIG. 5B is a graph depicting the body weight change of the H446 mouse tumor model upon administration of the LNP composition.

FIG. 6A is a graph depicting the results of a H446 mouse tumor model showing the growth of tumor upon repeat dose of the LNP compositions of the disclosure. FIG. 6B is a graph depicting the body weight change of the H446 mouse tumor model upon administration of the LNP composition.

FIG. 7A is a graph depicting the results of a dynamic light scattering experiment of LNP compositions comprising Brij S100 or Myrj S40. FIG. 7B is a graph depicting the encapsulation efficiency of these LNP compositions measured by RiboGreen.

FIG. 8A and FIG. 8C depict the results of a SK-MEL-28 mouse tumor model showing the growth of tumor upon repeat dose of the LNP compositions of the disclosure. FIG. 8B and FIG. 8D depict the body weight change of the SK-MEL-28 mouse tumor model upon administration of the LNP composition of the disclosure. FIG. 8E shows Rt-qPCR measurements for CVA21 replication.

FIG. 9 shows a schematic representation of LNP/picornavirus RNA composition and mode of action. LNP/picornavirus RNA is systemically administered, and picornavirus RNA genomes are delivered to permissive tumor cells where they replicate and produce picornavirus virions. Picomavirus infection then spreads to neighboring tumor cells eliciting oncolysis and antiviral immune responses.

FIG. 10A and FIG. 10B depict the particle sizes (FIG. 10A) and polydispersity index (FIG. 10B) determined in a dynamic light scattering experiment of LNP compositions. FIG. 10C depicts the encapsulation efficiency of these LNP compositions measured by RiboGreen.

FIG. 11A and FIG. 11B depict the particle sizes (FIG. 11A) and polydispersity index (FIG. 11B) determined in a dynamic light scattering experiment of LNPs encapsulating SVV-RNA purified either via tangential flow filtration (TFF) or via oligo-dT chromatography and reverse phase chromatography. FIG. 11C depicts the encapsulation efficiency of these LNP compositions measured by RiboGreen.

FIG. 12A and FIG. 12B depict the particle sizes (FIG. 12A) and polydispersity index (FIG. 12B) determined in a dynamic light scattering experiment of CAT4 and CAT5 LNP compositions made with various RNA acidifying buffers. FIG. 12C depicts the encapsulation efficiency of these LNP compositions measured by RiboGreen.

FIG. 13A and FIG. 13B depict the particle sizes (FIG. 13A) and polydispersity index (FIG. 13B) determined in a dynamic light scattering experiment of LNP compositions. FIG. 13C depicts the encapsulation efficiency of these LNP compositions measured by RiboGreen.

FIG. 14A depicts the particle sizes determined in a dynamic light scattering experiment (left) and encapsulation efficiency measured by RiboGreen (right) of LNP compositions stored at −20° C.

FIG. 14B depicts the particle sizes determined in a dynamic light scattering experiment (left) and encapsulation efficiency measured by RiboGreen (right) of LNP compositions stored at −80° C.

FIG. 15 shows a schematic representation of the formulation process for the LNP formulations.

FIG. 16A, FIG. 16B, and FIG. 16C depict the RNA levels measured by the luminescence produced by NanoLuc luciferase activation 96 h post-dose of the respective LNP formulations.

FIG. 16D, FIG. 16E, and FIG. 16F depict the RNA levels measured by the luminescence produced by NanoLuc luciferase activation 72 h post-dose of the respective LNP formulations.

FIGS. 17A-17E depict the tumor volume (left) and body weight change (right) over the days of treatment of the mice treated with the respective LNP formulations.

FIG. 18A depicts the RNA levels measured by the luminescence produced by NanoLuc luciferase activation 72 h post-dose of the respective LNP formulations. FIG. 18B depicts the tumor volume (right) and body weight change (left) over the days of treatment of the mice treated with the respective LNP formulations.

FIGS. 19A-19E depict the concentration of the ionizable lipid comprised in the LNPs (SS—OC) in the plasma of the treated mice measured by LC-MS.

FIGS. 20A-20D depict the concentration of the ionizable lipid comprised in the LNPs (SS—OC) in the plasma of the treated mice measured by LC-MS.

FIGS. 21A-21F depict the concentration of the ionizable lipid comprised in the LNPs (SS—OC or CAT7) in the plasma of the treated mice measured by LC-MS.

FIGS. 22A-22E depict the concentration of the ionizable lipid comprised in the LNPs (SS—OC, CAT7, or CAT11) in the plasma of the treated mice measured by LC-MS.

FIG. 23A and FIG. 23B depict the IgM levels at the indicated timepoints of the mice treated with the respective LNP formulations measured by an ELISA assay.

FIG. 24A and FIG. 24B depict the IgG levels at the indicated timepoints of the mice treated with the respective LNP formulations measured by an ELISA assay.

FIG. 25A and FIG. 25B depict the plasma levels of the mRNA BiTE (FIG. 25A) or hEPO (FIG. 25B) measure by ECL assays.

FIG. 26 depicts an A-optimal design of screening experiments for LNPs comprising CAT7.

FIG. 27 shows the prediction profilers modeled based on the design of experiment runs for LNPs comprising CAT7 and using the Self-Validated Ensemble Modeling method.

### DETAILED DESCRIPTION

### Definitions

### Chemical Definitions

The term “aliphatic” or “aliphatic group,” as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “cycloaliphatic,” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

The term “alkyl” as used herein is a branched or unbranched saturated hydrocarbon group having a specified number of carbon atoms. In some embodiments, alkyl refers to a branched or unbranched saturated hydrocarbon group having three carbon atoms (C₃). In some embodiments, alkyl refers to a branched or unbranched saturated hydrocarbon group having six carbon atoms (C₆). In some embodiments, the term “alkyl” includes, but is not limited to, methyl, ethyl, n-propyl, isopropyl, n-butyl, isobutyl, s-butyl, t-butyl, n-pentyl, isopentyl, s-pentyl, neopentyl, and hexyl.

As used herein, the term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “aryl” used alone or as part of a larger moiety as in “aralkyl,” “aralkoxy,” or “aryloxyalkyl,” refers to monocyclic and bicyclic ring systems having a total of five to fourteen ring members, wherein at least one ring in the system is aromatic and wherein each ring in the system contains three to seven ring members. The term “aryl” may be used interchangeably with the term “aryl ring”. In certain embodiments of the present disclosure, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14 μL electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-,” as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

The term “haloaliphatic” refers to an aliphatic group that is substituted with one or more halogen atoms.

The term “haloalkyl” refers to a straight or branched alkyl group that is substituted with one or more halogen atoms.

The term “halogen” means F, Cl, Br, or I.

As used herein, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in TV-substituted pyrrolidinyl). A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl, where the radical or point of attachment is on the heterocyclyl ring. A heterocyclyl group may be mono- or bicyclic. The term “heterocyclylalkyl” refers to an alkyl group substituted by a heterocyclyl, wherein the alkyl and heterocyclyl portions independently are optionally substituted.

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl, where the radical or point of attachment is on the heterocyclyl ring. A heterocyclyl group may be mono- or bicyclic. The term “heterocyclylalkyl” refers to an alkyl group substituted by a heterocyclyl, wherein the alkyl and heterocyclyl portions independently are optionally substituted.

As described herein, compounds of the disclosure may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this disclosure are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{o}; —(CH₂)₀₋₄OR^{o}; —O(CH₂)₀₋₄R^{o}, —O—(CH₂)₀₋₄C(O)OR^{o}; —(CH₂)₀₋₄CH(OR^{o})₂; —(CH₂)₀₋₄SR^{o}; —(CH₂)₀₋₄Ph, which may be substituted with R^{o}; —(CH₂)₀₋₄O(CH₂)₀₋₁Ph which may be substituted with R^{o}; —CH═CHPh, which may be substituted with R^{o}; —(CH₂)₀₋₄O(CH₂)₀₋₁-pyridyl which may be substituted with R^{o}; —NO₂; —CN; —N₃; —(CH₂)₀₋₄N(R^{o})₂; —(CH₂)₀₋₄N(R^{o})C(O)R^{o}; —N(R^{o})C(S)R^{o}; —(CH₂)₀₋₄N(R^{o})C(O)NR^{o}2; —N(R^{o})C(S)NR^{o}2; —(CH₂)₀₋₄N(R^{o})C(O)OR^{o}; —N(R^{o})N(R^{o})C(O)R^{o}; —N(R^{o})N(R^{o})C(O)NR^{o}2; —N(R^{o})N(R^{o})C(O)OR^{o}; —(CH₂)₀₋₄C(O)R^{o}; —C(S)R^{o}; —(CH₂)₀₋₄C(O)OR^{o}; —(CH₂)₀₋₄C(O)SR^{o}; —(CH₂)₀₋₄C(O)OSiR^{o}₃; —(CH₂)₀₋₄OC(O)R^{o}; —OC(O)(CH₂)₀₋₄SR^{o}, SC(S)SR^{o}; —(CH₂)₀₋₄SC(O)R^{o}; —(CH₂)₀₋₄C(O)NR^{o}2; —C(S)NR^{o}2; —C(S)SR^{o}; —SC(S)SR^{o}, —(CH₂)₀₋₄OC(O)NR^{o}2; —C(O)N(OR^{o})R^{o}; —C(O)C(O)R^{o}; —C(O)CH₂C(O)R^{o}; —C(NOR^{o})R^{o}; —(CH₂)₀₋₄SSR^{o}; —(CH₂)₀₋₄S(O)₂R^{o}; —(CH₂)₀₋₄S(O)₂OR^{o}; —(CH₂)₀₋₄OS(O)₂R^{o}; —S(O)₂NR^{o}2; —(CH₂)₀₋₄S(O)R^{o}; —N(R^{o})S(O)₂NR^{o}2; —N(R^{o})S(O)₂R^{o}; —N(OR^{o})R^{o}; —C(NH)NR^{o}2; —P(O)₂R^{o}; —P(O)R^{o}2; —OP(O)R^{o}2; —OP(O)(OR^{o})₂; SiR^{o}3; —(C₁₋₄ straight or branched alkylene)O—N(R^{o})₂; or —(C₁-4 straight or branched alkylene)C(O)O—N(R^{o})₂, wherein each R^{o} may be substituted as defined below and is independently hydrogen, C₁-6 aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{o}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{o} (or the ring formed by taking two independent occurrences of R^{o} together with their intervening atoms), are independently halogen, —(CH₂)₀₋₂R*, ˜(haloR*), —(CH₂)₀₋₂OH, —(CH₂)₀₋₂OR*, —(CH₂)₀₋₂CH(OR*)₂; —O(haloR*), —CN, —N₃, —(CH₂)₀₋₂C(O)R*, —(CH₂)₀₋₂C(O)OH, —(CH₂)₀₋₂C(O)OR*, —(CH₂)₀₋₂SR*, —(CH₂)₀₋₂SH, —(CH₂)₀₋₂NH₂, —(CH₂)₀₋₂NHR*, —(CH₂)₀₋₂NR′ 2, —NO₂, —SiR*₃, —OSiR*₃, —C(O)SR*, —(C₁₋₄ straight or branched alkylene)C(O)OR*, or —SSR′ wherein each R′ is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{o} include ═O and ═S.

Suitable divalent substituents on a saturated carbon atom of an “optionally substituted” group include the following: ═O, ═S, ═NNR*2, ═NNHC(O)R*, ═NNHC(O)OR*, ═NNHS(O)₂R*, ═NR*, NOR*, —O(C(R*2))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*2)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R*, -(haloR*), —OH, —OR*, —O(haloR*), —CN, —C(O)OH, —C(O)OR*, —NH₂, —NHR*, —NR*₂, or —NO₂, wherein each R* is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{t}, —NR^{t}₂, —C(O)R^{t}, —C(O)OR^{t}, —C(O)C(O)R^{t}, —C(O)CH₂C(O)R^{t}, —S(O)₂R, —S(O)₂NR^{t}₂, —C(S)NR^{t}₂, —C(NH)NR^{t}₂, or —N(R^{t})S(O)₂R; wherein each R^{t} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{t}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R are independently halogen, —R*, -(haloR*), —OH, —OR*, —O(haloR*), —CN, —C(O)OH, —C(O)OR*, —NH₂, —NHR*, —NR*₂, or —NO₂, wherein each R* is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

As used herein, the term “partially unsaturated” refers to a ring moiety that includes at least one double or triple bond. The term “partially unsaturated” is intended to encompass rings having multiple sites of unsaturation but is not intended to include aryl or heteroaryl moieties, as herein defined.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

A “pharmaceutically acceptable derivative” means any non-toxic salt, ester, salt of an ester or other derivative of a compound of this disclosure that, upon administration to a recipient, is capable of providing, either directly or indirectly, a compound of this disclosure or an active metabolite or residue thereof.

The term “tertiary amine” is used to describe an amine (nitrogen atom) which is attached to three carbon-containing groups, each of the groups being covalently bonded to the amine group through a carbon atom within the group. A tertiary amine may be protonated or form a complex with a Lewis acid.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof.

Unless otherwise stated, structures depicted herein are also meant to include all enantiomeric, diastereomeric, and geometric (or conformational) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the present disclosure. Unless otherwise stated, all tautomeric forms of the compounds of the present disclosure are within the scope of the present disclosure.

### Other Definitions

As used in this specification and the appended claims, the singular forms “a,” “an,” and “the” include plural references unless the content clearly dictates otherwise.

The term “about” is used herein to mean approximately, in the region of, roughly, or around. When the term “about” is used in conjunction with a numerical range, it modifies that range by extending the boundaries above and below the numerical values set forth. Accordingly, the term “about” means a quantity, level, value, number, frequency, percentage, dimension, size, amount, weight, or length that varies by acceptable levels in the art. In some embodiments, such variation may be as much as 30, 25, 20, 15, 10, 9, 8, 7, 6, 5, 4, 3, 2, or 1% to a reference quantity, level, value, number, frequency, percentage, dimension, size, amount, weight or length. In some embodiments, such variation may be as much as 10% to a reference quantity, level, value, number, frequency, percentage, dimension, size, amount, weight or length.

The term “accelerated blood clearance” or “ABC” refers to a phenomenon in which certain pharmaceutical agents (e.g., PEG-containing LNPs) are rapidly cleared from the blood upon second and subsequent administrations. ABC has been observed for many lipid-delivery vehicles, including liposomes and LNPs.

The term “administration” refers herein to introducing a composition into a subject or contacting a composition with a cell and/or tissue.

As used in this specification, the term “and/or” is used in this disclosure to either “and” or “or” unless indicated otherwise.

The term “antibody” refers to an immunoglobulin (Ig) molecule capable of binding to a specific target, such as a carbohydrate, polynucleotide, lipid, or polypeptide, through at least one epitope recognition site located in the variable region of the Ig molecule. As used herein, the term encompasses intact polyclonal or monoclonal antibodies and antigen-binding fragments thereof. For example, a native immunoglobulin molecule is comprised of two heavy chain polypeptides and two light chain polypeptides. Each of the heavy chain polypeptides associate with a light chain polypeptide by virtue of interchain disulfide bonds between the heavy and light chain polypeptides to form two heterodimeric proteins or polypeptides (i.e., a protein comprised of two heterologous polypeptide chains). The two heterodimeric proteins then associate by virtue of additional interchain disulfide bonds between the heavy chain polypeptides to form an immunoglobulin protein or polypeptide.

The term “cancer” refers to or describes the physiological condition in mammals that is typically characterized by unregulated cell growth.

As used herein, the term “combination,” “combined,” and related terms refers to the simultaneous or sequential administration of therapeutic agents in accordance with this disclosure. For example, a compound of the present disclosure may be administered with another therapeutic agent simultaneously or sequentially in separate unit dosage forms or together in a single unit dosage form. Accordingly, the present disclosure provides a single unit dosage form comprising a provided compound, an additional therapeutic agent, and a pharmaceutically acceptable carrier, adjuvant, or vehicle.

The term “biological sample,” as used herein, includes, without limitation, cell cultures or extracts thereof; biopsied material obtained from a mammal or extracts thereof; and blood, saliva, urine, feces, semen, tears, or other body fluids or extracts thereof. Examples of such purposes include, but are not limited to, blood transfusion, organ transplantation, biological specimen storage, and biological assays.

The expression “dosage unit form” as used herein refers to a physically discrete unit of agent appropriate for the patient to be treated. It will be understood, however, that total daily usage of compounds and compositions of the present disclosure will be decided by the attending physician within the scope of sound medical judgment. Specific effective dose level for any particular patient or organism will depend upon a variety of factors including disorder being treated and severity of the disorder; activity of specific compound employed; specific composition employed; age, body weight, general health, sex and diet of the patient; time of administration, route of administration, and rate of excretion of a specific compound employed; duration of treatment; drugs used in combination or coincidental with a specific compound employed, and like factors well known in the medical arts.

The term “encapsulation efficiency” or “EE %” refers to the percentage of payload that is successfully entrapped into LNP. In some embodiments, encapsulation efficiency may be calculated using the formula:

(EE %)=(Wt/Wi)×100%

where Wt is the total amount of drug in the LNP suspension and Wi is the total quantity of drug added initially during preparation. As an illustrative example, if 97 mg of the payload molecule are entrapped into LNPs out of a total 100 mg of the payload molecule initially provided to the composition, the encapsulation efficiency may be given as 97%.

The term “half-life” refers to a pharmacokinetic property of a payload molecule (e.g., a payload molecule encapsulated in a lipid nanoparticle). Half-life can be expressed as the time required to eliminate through biological processes (e.g., metabolism, excretion, accelerated blood clearance, etc.) fifty percent (50%) of a known quantity of the payload molecules in vivo, following their administration, from the subject's body (e.g., human patient or other mammal) or a specific compartment thereof, for example, as measured in serum, i.e., circulating half-life, or in other tissues. In general, an increase in half-life results in an increase in mean residence time (MRT) in circulation for the payload molecule administered.

The term “lipid-nitrogen-to-phosphate ratio” or “(N:P)” refers to the ratio of positively-chargeable lipid amine groups to nucleic acid phosphate groups in a lipid nanoparticle.

As used herein, “nucleic acid” means a polynucleotide or oligonucleotide and includes a single or a double-stranded polymer or oligomer of deoxyribonucleotide or ribonucleotide bases. Nucleic acids may also include fragments and modified nucleotides. Thus, the terms “polynucleotide,” “oligonucleotide,” “nucleic acid sequence,” “nucleotide sequence” and “nucleic acid fragment” are used interchangeably to denote a polymer or oligomer of RNA and/or DNA that is single- or double-stranded, optionally containing synthetic, non-natural, or altered nucleotide bases. Nucleotides (usually found in their 5′-monophosphate form) may be referred to by their single letter designation as commonly known in the art.

As used herein, a polypeptide or polynucleotide from which another polypeptide or polynucleotide is derived from is referred to as the “parental” or “reference” polynucleotide or polypeptide.

As used herein, the term “pharmaceutically acceptable” refers to molecular entities and compositions that do not generally produce allergic or other serious adverse reactions when administered using routes well known in the art. Molecular entities and compositions approved by a regulatory agency of the Federal or a state government or listed in the U.S. Pharmacopeia or other generally recognized pharmacopeia for use in animals, and more particularly in humans are considered to be “pharmaceutically acceptable.”

The term “pharmaceutically acceptable carrier, adjuvant, or vehicle” refers to a non-toxic carrier, adjuvant, or vehicle that does not destroy the pharmacological activity of the compound(s) with which it is formulated. Pharmaceutically acceptable carriers, adjuvants or vehicles that may be used in the compositions of the compounds disclosed herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The term “polynucleotide” as referred to herein means single-stranded or double-stranded nucleic acid polymers. In some embodiments, the nucleotides comprising the polynucleotide can be RNA or DNA or a modified form of either type of nucleotide, including a modified messenger RNA, transfer RNA, and small RNA. Said modifications may include, but are not limited to, base modifications such as bromouridine, ribose modifications such as arabinoside and 2′,3′-dideoxyribose and internucleotide linkage modifications such as phosphorothioate, phosphorodithioate, phosphoroselenoate, phosphorodiselenoate, phosphoroanilothioate, phoshoraniladate and phosphoroamidate. The term “polynucleotide” also includes single and double stranded forms when refers to DNA.

The terms “polypeptide” and “protein” are used interchangeably herein and refer to a single, linear, and contiguous arrangement of covalently linked amino acids. Polypeptides can form one or more intrachain disulfide bonds.

As used herein, “prophylaxis” can mean complete prevention of the symptoms of a disease, a delay in onset of the symptoms of a disease, or a lessening in the severity of subsequently developed disease symptoms. Typically, but not necessarily, since a prophylactic dose is used in subjects prior to or at an earlier stage of disease, the prophylactically effective amount is less than the therapeutically effective amount.

A “response” to a method of treatment can include a decrease in or amelioration of negative symptoms, a decrease in the progression of a disease or symptoms thereof, an increase in beneficial symptoms or clinical outcomes, a lessening of side effects, stabilization of disease, partial or complete remedy of disease, among others.

As used herein, the term “sequence identity” refers to a relationship between two or more polynucleotide sequences or between two or more polypeptide sequences. When a position in one sequence is occupied by the same nucleic acid base or amino acid residue in the corresponding position of the comparator sequence, the sequences are said to be “identical” at that position. The percentage sequence identity is calculated by determining the number of positions at which the identical nucleic acid base or amino acid residue occurs in both sequences to yield the number of identical positions. The number of identical positions is then divided by the total number of positions in the comparison window and multiplied by 100 to yield the percentage of sequence identity. Percentage of sequence identity is determined by comparing two optimally aligned sequences over a comparison window. The comparison window for polynucleotide sequences can be, for instance, at least 20, 30, 40, 50, 60, 70, 80, 90, 100, 110, 120, 130, 140, 150, 160, 170, 180, 190, 200, 300, 400, 500, 600, 700, 800, 900 or 1000 or more nucleic acids in length. The comparison window for polypeptide sequences can be, for instance, at least 20, 30, 40, 50, 60, 70, 80, 90, 100, 110, 120, 130, 140, 150, 160, 170, 180, 190, 200, 300 or more amino acids in length. In order to optimally align sequences for comparison, the portion of a polynucleotide or polypeptide sequence in the comparison window can comprise additions or deletions termed gaps while the reference sequence is kept constant. An optimal alignment is that alignment which, even with gaps, produces the greatest possible number of “identical” positions between the reference and comparator sequences. Percentage “sequence identity” between two sequences can be determined using the version of the program “BLAST 2 Sequences” which was available from the National Center for Biotechnology Information as of Sep. 1, 2004, which program incorporates the programs BLASTN (for nucleotide sequence comparison) and BLASTP (for polypeptide sequence comparison), which programs are based on the algorithm of Karlin and Altschul (Proc. Natl. Acad. Sci. USA 90(12):5873-5877, 1993). When utilizing “BLAST 2 Sequences,” parameters that were default parameters as of Sep. 1, 2004, can be used for word size (3), open gap penalty (11), extension gap penalty (1), gap dropoff (50), expect value (10) and any other required parameter including but not limited to matrix option. Two nucleotide or amino acid sequences are considered to have “substantially similar sequence identity” or to be “substantially identical” if the two sequences have at least 80%, at least 85%, at least 90%, at least 95%, at least 96%, at least 97%, at least 98%, or at least 99% sequence identity relative to each other.

The term “subject” or “patient” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or other primates (e.g., cynomolgus monkeys, rhesus monkeys); mammals, including commercially relevant mammals such as cattle, pigs, horses, sheep, goats, cats, and/or dogs; and/or birds, including commercially relevant birds such as chickens, ducks, geese, quail, and/or turkeys. Preferred subjects are humans.

As used herein, a “therapeutically effective amount” means an amount of a substance (e.g., a therapeutic agent, composition, and/or formulation) that elicits a desired biological response. In some embodiments, a therapeutically effective amount of a substance is an amount that is sufficient, when administered as part of a dosing regimen to a subject suffering from or susceptible to a disease, disorder, and/or condition, to treat and/or diagnose the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the therapeutically effective amount of the LNP and compositions thereof described herein will depend on the condition to be treated the severity and course of the condition, whether the LNP or the composition thereof is administered for preventive or therapeutic purposes, previous therapy, the subject's clinical history and response to the LNP or the composition thereof used, and the discretion of the attending physician. In some embodiments, the effective amount of provided LNPs or compositions thereof to treat a disease, disorder, and/or condition is the amount that alleviates, ameliorates, relieves, reduces severity of and/or reduces incidence of one or more symptoms or features of the disease, disorder, and/or condition. In some embodiments, a “therapeutically effective amount” is at least a minimal amount of a provided compound, or composition containing a provided compound, which is sufficient for treating one or more symptoms of a disease or disorder.

The terms “treat,” “treatment” or “treating” mean to decrease, suppress, attenuate, diminish, arrest, or stabilize the development or progression of a disease (e.g., a disease or disorder delineated herein), lessen the severity of the disease or improve the symptoms associated with the disease. Treatment includes treating a symptom of a disease, disorder or condition. If it is administered prior to clinical manifestation of the unwanted condition (e.g., disease or other unwanted state of the subject) then the treatment is prophylactic (i.e., it protects the subject against developing the unwanted condition), whereas if it is administered after manifestation of the unwanted condition, the treatment is therapeutic, (i.e., it is intended to diminish, ameliorate, or stabilize the existing unwanted condition or side effects thereof).

The term “variant” or “variants” as used herein refers to a polynucleotide or polypeptide with a sequence differing from that of a reference polynucleotide or polypeptide, but retaining essential properties of the parental polynucleotide or polypeptide. Generally, variant polynucleotide or polypeptide sequences are overall closely similar, and, in many regions, identical to the parental polynucleotide or polypeptide. For instance, a variant polynucleotide or polypeptide may exhibit at least 70%, at least 80%, at least 90%, at least 91%, at least 92%, at least 93%, at least 94%, at least 95%, at least 96%, at least 97%, at least 98% at least 99%, or at least 99.5% sequence identity compared to the parental polynucleotide or polypeptide.

### Compounds

### Compounds of Formula (I)

In various embodiments provided herein are compounds of Formula (I):

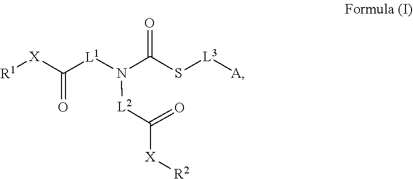

or a pharmaceutically acceptable salt or solvate thereof, wherein:
A is —N(CH₂R^{N1})(CH₂R^{N2}) or a 4-7-membered heterocyclyl ring containing at least one N, wherein the 4-7-membered heterocyclyl ring is optionally substituted with 0-6 R³;
each X is independently —O—, —N(R′)—, or —N(R²)—
R¹ is selected from the group consisting of optionally substituted C₁-C₃₁ aliphatic and steroidyl;
R² is selected from the group consisting of optionally substituted C₁-C₃₁ aliphatic and steroidyl;
R³ is optionally substituted C₁-C₆ aliphatic;
R^{N1} and R^{N2} are each independently hydrogen, hydroxy-C₁-C₆ alkyl, C₂-C₆ alkenyl, or a C₃-C₇ cycloalkyl;
L¹ is selected from the group consisting of an optionally substituted C₁-C₂₀ alkylene chain and a bivalent optionally substituted C₂-C₂₀ alkenylene chain;
L² is selected from the group consisting of an optionally substituted C₁-C₂₀ alkylene chain and a bivalent optionally substituted C₂-C₂₀ alkenylene chain;
L³ is a bond, an optionally substituted C₁-C₆ alkylene chain, or a bivalent optionally substituted C₃-C₇ cycloalkylene.

In some embodiments, when A is —N(CH₃)(CH₃) and X is 0, L³ is not a C₁-C₆ alkylene chain.

In some embodiments, the present disclosure includes a compound of Formula (I-a):

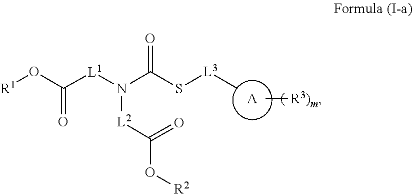

or a pharmaceutically acceptable salt or solvate thereof, wherein m is 0, 1, 2, 3, 4, 5, or 6.

In some embodiments, the present disclosure includes a compound of Formula (I-b):

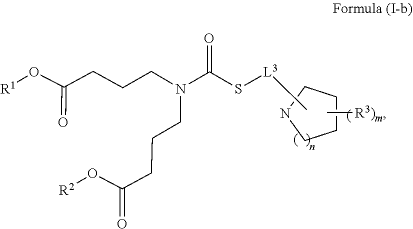

or a pharmaceutically acceptable salt or solvate thereof, wherein n is 0, 1, 2, or 3; and m is 0, 1, 2, 3, 4, 5, or 6.

In some embodiments, the present disclosure includes a compound of Formula (I-bi):

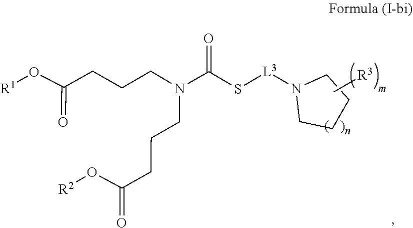

or a pharmaceutically acceptable salt or solvate thereof.

In some embodiments, the present disclosure includes a compound of Formula (I-bii):

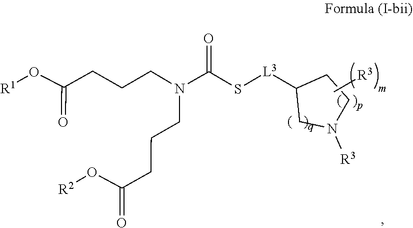

or a pharmaceutically acceptable salt or solvate thereof, wherein m is 0, 1, 2, or 3; and p and q are each 0, 1, 2, or 3, and wherein q+p is less than or equal to 3.

In some embodiments, the present disclosure includes a compound of Formula (I-biii):

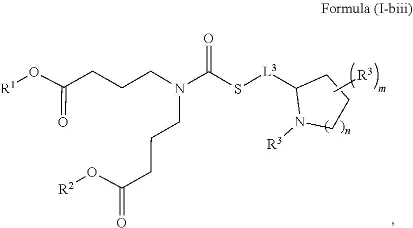

or a pharmaceutically acceptable salt or solvate thereof.

In some embodiments, the present disclosure includes a compound of Formula (I-c):

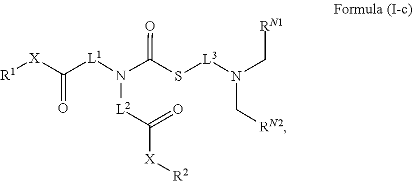

or a pharmaceutically acceptable salt or solvate thereof.

In some embodiments, A is —N(CH₂R^{N1})(CH₂R^{N2}) or an optionally substituted 4-7-membered heterocyclyl ring containing at least one N.

In some embodiments, A is —N(CH₂R^{N1})(CH₂R^{N2}). In some embodiments, R^{N1} and R^{N2} are each independently selected from hydrogen, hydroxy-C₁-C₃ alkylene, C₂-C₄ alkenyl, or C₃-C₄ cycloalkyl.).

In some embodiments, R^{N1} and R^{N2} are each independently selected from hydrogen, —CH₂CH═CH₂, —CH₂CH₂OH

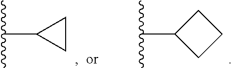

In some embodiments, R^{N1} and R^{N2} are the same. In some embodiments, R^{N1} and R^{N2} are each hydrogen. In some embodiments, R^{N1} and R^{N2} are each C₂-C₄ alkenyl, e.g., —CH₂CH═CH₂. In some embodiments, R^{N1} and R^{N2} are each hydroxy-C₁-C₃ alkylene, e.g., —CH₂CH₂OH. In some embodiments, R^{N1} and R^{N2} are different. In some embodiments, one of R^{N1} and R^{N2} is hydrogen and the other one is C₃-C₄ cycloalkyl. In some embodiments, one of R^{N1} and R^{N2} is hydrogen and the other one is

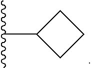 [SMILES: *C1CCC1]

In some embodiments, A is an optionally substituted 4-7-membered heterocyclyl ring containing at least one N. In some embodiments, A is an optionally substituted 4-7-membered heterocyclyl ring containing exactly one N. In some embodiments, A is an unsubstituted 4-7-membered heterocyclyl ring containing at least one N. In some embodiments, A is unsubstituted 4-7-membered heterocyclyl ring containing exactly one N. In some embodiments, A is an optionally substituted 5-6-membered heterocyclyl ring containing at least one N. In some embodiments, A is unsubstituted 5-6-membered heterocyclyl ring containing at least one N.

In some embodiments, A is an optionally substituted 4-7-membered heterocyclyl ring containing at least one N, and the N atom of A is a tertiary amine.

In some embodiments, A is an optionally substituted 4-7-membered heterocyclyl ring containing at least one N, further containing one or more S. In some embodiments, A is an optionally substituted 4-7-membered heterocyclyl ring containing at least one N, further containing exactly one S.

In some embodiments, A is selected from the group consisting of azetidine, pyrrolidine, piperidine, azepane, and thiomorpholine. In some embodiments, A is selected from the group consisting of pyrrolidine and piperidine.

In some embodiments, L¹ is selected from the group consisting of an optionally substituted C₁-C₂₀ alkylene chain and a bivalent optionally substituted C₁-C₂₀ alkenylene chain. In some embodiments, L² is selected from the group consisting of an optionally substituted C₁—C₂₀ alkylene chain and a bivalent optionally substituted C₁-C₂₀ alkenylene chain. In some embodiments, L¹ is an optionally substituted C₁-C₂₀ alkylene chain. In some embodiments, L² is an optionally substituted C₁-C₂₀ alkylene chain.

In some embodiments, L¹ and L² are the same. In some embodiments, L¹ and L² are different.

In some embodiments, L¹ is an optionally substituted C₁-C₁₀ alkylene chain. In some embodiments, L² is an optionally substituted C₁-C₁₀ alkylene chain. In some embodiments, L¹ is an optionally substituted C₁-C₅ alkylene chain. In some embodiments, L² is an optionally substituted C₁-C₅ alkylene chain.

In some embodiments, L¹ and L² are each —CH₂CH₂CH₂CH₂—. In some embodiments, L¹ and L² are each —CH₂CH₂CH₂—. In some embodiments, L¹ and L² are each —CH₂CH₂—.

In some embodiments, L³ is a bond, an optionally substituted C₁-C₆ alkylene chain, or a bivalent optionally substituted C₃-C₆ cycloalkylene. In some embodiments, L³ is a bond. In some embodiments, L³ is an optionally substituted C₁-C₆ alkylene chain. In some embodiments, L³ is an optionally substituted C₁-C₃ alkylene chain. In some embodiments, L³ is an unsubstituted C₁-C₃ alkylene chain. In some embodiments, L³ is —CH₂—. In some embodiments, L³ is —CH₂CH₂—. In some embodiments, L³ is —CH₂CH₂CH₂—. In some embodiments, L³ is a bivalent C₃-C₆ cyclcoalkylene. In some embodiments, L³ is

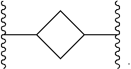 [SMILES: *C1CC(*)C1]

In some embodiments, the number of carbon atoms between the S of the thiolate of Formula (I) and the N of A is 2-10. In some embodiments, the number of carbon atoms between the S of the thiolate of Formula (I) and the N of A is 2-8. In some embodiments, the number of carbon atoms between the S of the thiolate of Formula (I) and the N of A is 2-5. In some embodiments, the number of carbon atoms between the S of the thiolate of Formula (I) and the N of A is 2-4. In some embodiments, the number of carbon atoms between the S of the thiolate of Formula (I) and the N of A is 2. In some embodiments, the number of carbon atoms between the S of the thiolate of Formula (I) and the N of A is 3. In some embodiments, the number of carbon atoms between the S of the thiolate of Formula (I) and the N of A is 4.

In some embodiments, R¹ is selected from the group consisting of optionally substituted C₁-C₃₁ aliphatic and optionally substituted steroidyl. In some embodiments, R² is selected from the group consisting of optionally substituted C₁-C₃₁ aliphatic and optionally substituted steroidyl. In some embodiments, R¹ is optionally substituted C₁-C₃₁ alkyl. In some embodiments, R² is optionally substituted C₁-C₃₁ alkyl. In some embodiments, R¹ is optionally substituted C₅-C₂₅ alkyl. In some embodiments, R² is optionally substituted C₅-C₂₅ alkyl. In some embodiments, R¹ is optionally substituted C₁₀-C₂₀ alkyl. In some embodiments, R² is optionally substituted C₁₀-C₂₀ alkyl. In some embodiments, R¹ is optionally substituted C₁₀-C₂₀ alkyl. In some embodiments, R² is optionally substituted C₁₀-C₂₀ alkyl. In some embodiments, R¹ is unsubstituted C₁₀-C₂₀ alkyl. In some embodiments, R² is unsubstituted C₁₀-C₂₀ alkyl.

In some embodiments, R¹ is optionally substituted C₁₄-C₁₆ alkyl. In some embodiments, R² is optionally substituted C₁₄-C₁₆ alkyl. In some embodiments, R¹ is unsubstituted C₁₄-C₁₆ alkyl. In some embodiments, R² is unsubstituted C₁₄-C₁₆ alkyl.

In some embodiments, R¹ is optionally substituted branched C₃-C₃₁ alkyl. In some embodiments, R² is optionally substituted branched C₃-C₃₁ alkyl. In some embodiments, R¹ is optionally substituted branched C₁₀-C₂₀ alkyl. In some embodiments, R² is optionally substituted branched C₁₀-C₂₀ alkyl. In some embodiments, R¹ is optionally substituted branched C₁₄-C₁₆ alkyl. In some embodiments, R² is optionally substituted branched C₁₄-C₁₆ alkyl. In some embodiments, R¹ is substituted branched C₃-C₃₁ alkyl. In some embodiments, R² is substituted branched C₃-C₃₁ alkyl. In some embodiments, R¹ is substituted branched C₁₀-C₂₀ alkyl. In some embodiments, R² is substituted branched C₁₀-C₂₀ alkyl. In some embodiments, R¹ is substituted branched C₁₄-C₁₆ alkyl. In some embodiments, R² is substituted branched C₁₄-C₁₆ alkyl.

In some embodiments, R¹ and R² are the same.

In some embodiments, R¹ and R² are different. In some embodiments, R¹ is optionally substituted C₆-C₂₀ alkenyl and R² is optionally substituted C₁₀-C₂₀ alkyl. In some embodiments, R¹ is C₆-C₂₀ alkenyl and R² is branched C₁₀-C₂₀ alkyl.

In some embodiments, A is 4-7-membered heterocyclyl ring containing at least one N and optionally substituted with 0-6 R³. In some embodiments, R³ is optionally substituted C₁-C₆ aliphatic. In some embodiments, R³ is optionally substituted C₁-C₃ aliphatic. In some embodiments, R³ is optionally substituted C₁-C₆ alkyl. In some embodiments, R³ is optionally substituted C₁-C₃ alkyl. In some embodiments, R³ is unsubstituted C₁-C₆ alkyl. In some embodiments, R³ is unsubstituted C₁-C₃ alkyl. In some embodiments, R³ is optionally substituted C₁-C₆ alkenyl. In some embodiments, R³ is optionally substituted C₁-C₃ alkenyl. In some embodiments, R³ is unsubstituted C₁-C₆ alkenyl. In some embodiments, R³ is unsubstituted C₁-C₃ alkenyl.

In some embodiments, R³ is substitute with 1-3 C₃-C₆ cycloalkyl. In some embodiments, R³ is substitute with 1 C₃-C₆ cycloalkyl. In some embodiments, R³ is substitute with a cyclopropanyl. In some embodiments, R³ is substitute with 1-3 —OH. In some embodiments, R³ is substitute with 1 —OH.

In some embodiments, m is 0, 1, 2, 3, 4, 5, or 6. In some embodiments m is 0 or 1. In some embodiments, m is 0. In some embodiments, m is 1. In some embodiments, m is 2. In some embodiments, m is 3. In some embodiments, m is 4. In some embodiments, m is 5. In some embodiments, m is 6.

In some embodiments, n is 0, 1, 2, or 3. In some embodiments n is 1 or 2. In some embodiments, n is 0. In some embodiments, n is 1. In some embodiments, n is 2. In some embodiments, n is 3.

In some embodiments, a compound of Formula (I) is a compound selected from Table 1, or a pharmaceutically acceptable salt or solvate thereof.

TABLE 1

| Compound | |
|---|---|
| No. | Structure |
| CAT1 | 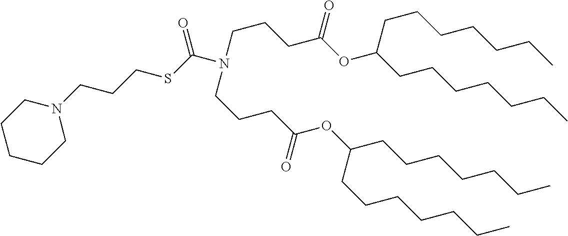 [SMILES: CCCCCCCC(CCCCCCC)OC(=O)CCCN(CCCC(=O)OC(CCCCCCC)CCCCCCC)C(=O)SCCCN1CCCCC1] |
| CAT2 | 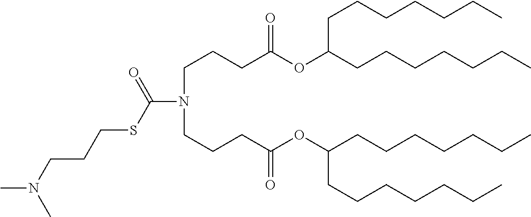 [SMILES: CCCCCCCC(CCCCCCC)OC(=O)CCCN(CCCC(=O)OC(CCCCCCC)CCCCCCC)C(=O)SCCCN(C)C] |
| CAT3 | 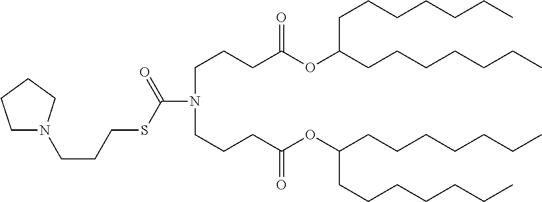 [SMILES: CCCCCCCC(CCCCCCC)OC(=O)CCCN(CCCC(=O)OC(CCCCCCC)CCCCCCC)C(=O)SCCCN1CCCC1] |
| CAT4 | 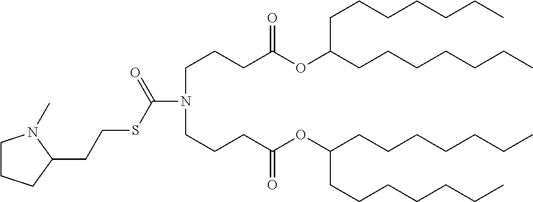 [SMILES: CCCCCCCC(CCCCCCC)OC(=O)CCCN(CCCC(=O)OC(CCCCCCC)CCCCCCC)C(=O)SCCC1CCCN1C] |
| CAT5 | 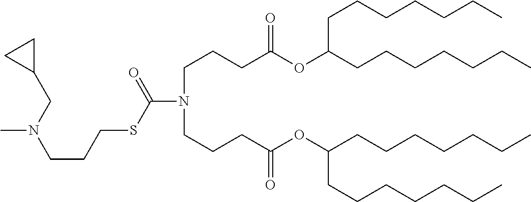 [SMILES: CCCCCCCC(CCCCCCC)OC(=O)CCCN(CCCC(=O)OC(CCCCCCC)CCCCCCC)C(=O)SCCCN(C)CC1CC1] |
| CAT6 | 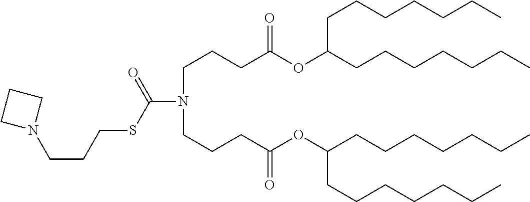 [SMILES: CCCCCCCC(CCCCCCC)OC(=O)CCCN(CCCC(=O)OC(CCCCCCC)CCCCCCC)C(=O)SCCCN1CCC1] |
| CAT7 | 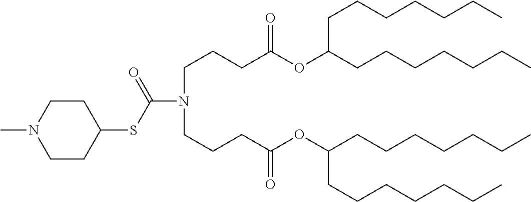 [SMILES: CCCCCCCC(CCCCCCC)OC(=O)CCCN(CCCC(=O)OC(CCCCCCC)CCCCCCC)C(=O)SC1CCN(C)CC1] |
| CAT8 | 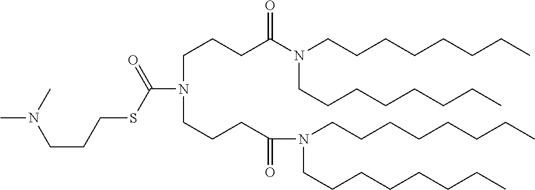 [SMILES: CCCCCCCCN(CCCCCCCC)C(=O)CCCN(CCCC(=O)N(CCCCCCCC)CCCCCCCC)C(=O)SCCCN(C)C] |
| CAT9 | 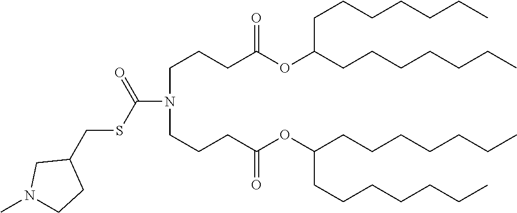 [SMILES: CCCCCCCC(CCCCCCC)OC(=O)CCCN(CCCC(=O)OC(CCCCCCC)CCCCCCC)C(=O)SCC1CCN(C)C1] |
| CAT10 | 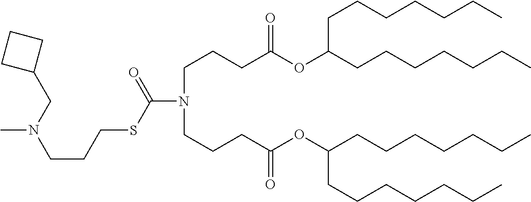 [SMILES: CCCCCCCC(CCCCCCC)OC(=O)CCCN(CCCC(=O)OC(CCCCCCC)CCCCCCC)C(=O)SCCCN(C)CC1CCC1] |
| CAT11 | 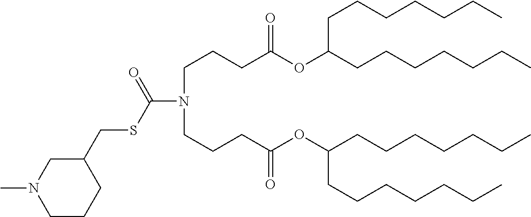 [SMILES: CCCCCCCC(CCCCCCC)OC(=O)CCCN(CCCC(=O)OC(CCCCCCC)CCCCCCC)C(=O)SCC1CCCN(C)C1] |
| CAT12 | 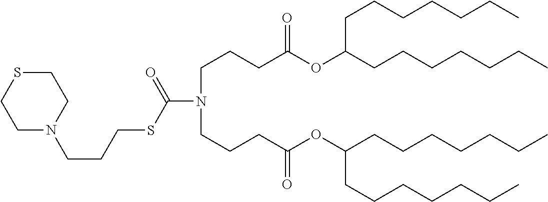 [SMILES: CCCCCCCC(CCCCCCC)OC(=O)CCCN(CCCC(=O)OC(CCCCCCC)CCCCCCC)C(=O)SCCCN1CCSCC1] |
| CAT13 | 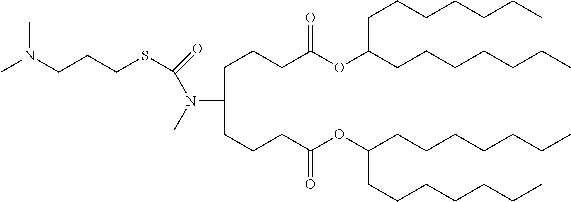 [SMILES: CCCCCCCC(CCCCCCC)OC(=O)CCCC(CCCC(=O)OC(CCCCCCC)CCCCCCC)N(C)C(=O)SCCCN(C)C] |
| CAT14 | 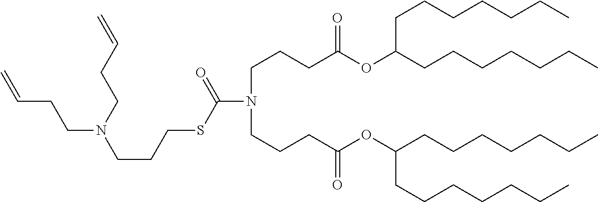 [SMILES: C=CCCN(CCC=C)CCCSC(=O)N(CCCC(=O)OC(CCCCCCC)CCCCCCC)CCCC(=O)OC(CCCCCCC)CCCCCCC] |
| CAT15 | 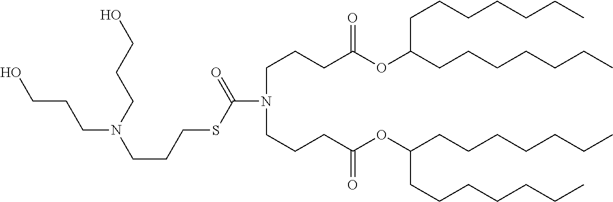 [SMILES: CCCCCCCC(CCCCCCC)OC(=O)CCCN(CCCC(=O)OC(CCCCCCC)CCCCCCC)C(=O)SCCCN(CCCO)CCCO] |
| CAT16 | 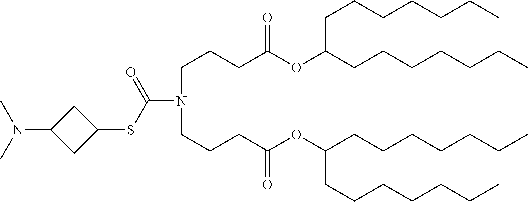 [SMILES: CCCCCCCC(CCCCCCC)OC(=O)CCCN(CCCC(=O)OC(CCCCCCC)CCCCCCC)C(=O)SC1CC(N(C)C)C1] |
| CAT17 | 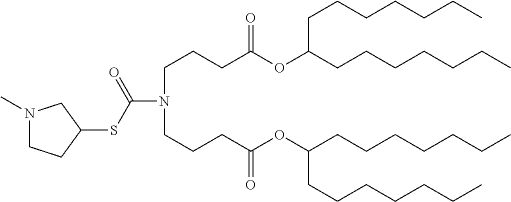 [SMILES: CCCCCCCC(CCCCCCC)OC(=O)CCCN(CCCC(=O)OC(CCCCCCC)CCCCCCC)C(=O)SC1CCN(C)C1] |
| CAT18 | 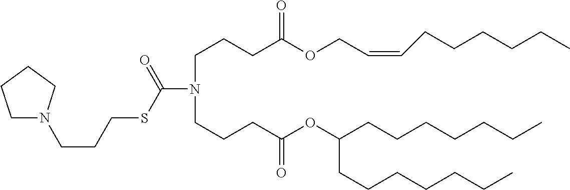 [SMILES: CCCCCC/C=C\COC(=O)CCCN(CCCC(=O)OC(CCCCCCC)CCCCCCC)C(=O)SCCCN1CCCC1] |
| CAT19 | 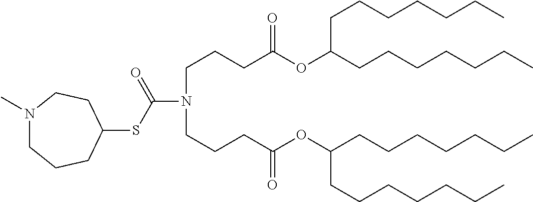 [SMILES: CCCCCCCC(CCCCCCC)OC(=O)CCCN(CCCC(=O)OC(CCCCCCC)CCCCCCC)C(=O)SC1CCCN(C)CC1] |
| CAT20 | 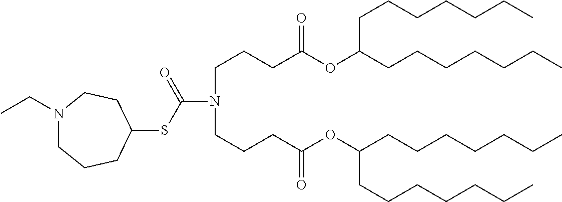 [SMILES: CCCCCCCC(CCCCCCC)OC(=O)CCCN(CCCC(=O)OC(CCCCCCC)CCCCCCC)C(=O)SC1CCCN(CC)CC1] |
| CAT21 | 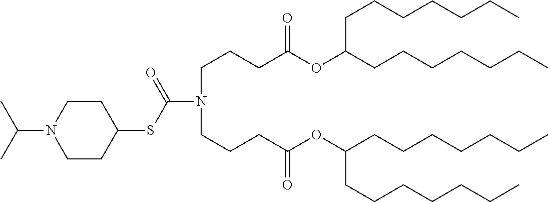 [SMILES: CCCCCCCC(CCCCCCC)OC(=O)CCCN(CCCC(=O)OC(CCCCCCC)CCCCCCC)C(=O)SC1CCN(C(C)C)CC1] |
| CAT22 | 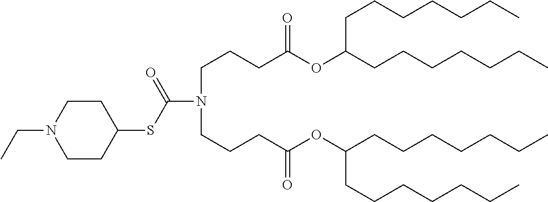 [SMILES: CCCCCCCC(CCCCCCC)OC(=O)CCCN(CCCC(=O)OC(CCCCCCC)CCCCCCC)C(=O)SC1CCN(CC)CC1] |
| CAT23 | 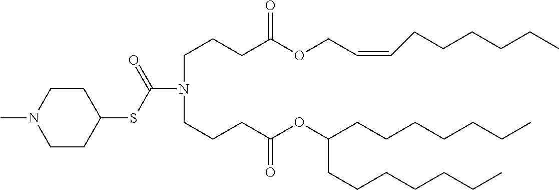 [SMILES: CCCCCC/C=C\COC(=O)CCCN(CCCC(=O)OC(CCCCCCC)CCCCCCC)C(=O)SC1CCN(C)CC1] |
| CAT24 | 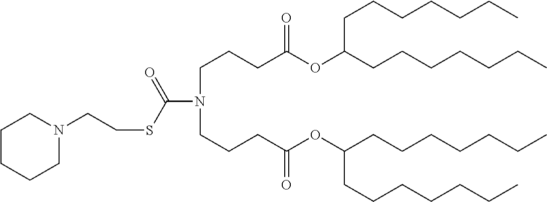 [SMILES: CCCCCCCC(CCCCCCC)OC(=O)CCCN(CCCC(=O)OC(CCCCCCC)CCCCCCC)C(=O)SCCN1CCCCC1] |
| CAT25 | 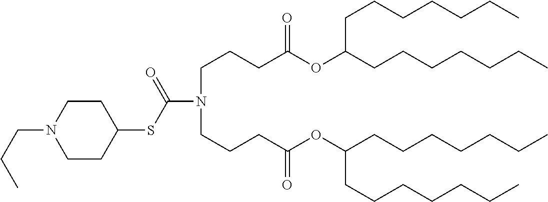 [SMILES: CCCCCCCC(CCCCCCC)OC(=O)CCCN(CCCC(=O)OC(CCCCCCC)CCCCCCC)C(=O)SC1CCN(CCC)CC1] |
| CAT26 | 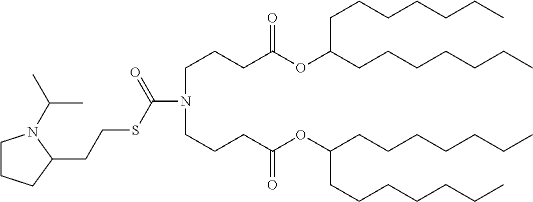 [SMILES: CCCCCCCC(CCCCCCC)OC(=O)CCCN(CCCC(=O)OC(CCCCCCC)CCCCCCC)C(=O)SCCC1CCCN1C(C)C] |
| CAT27 | 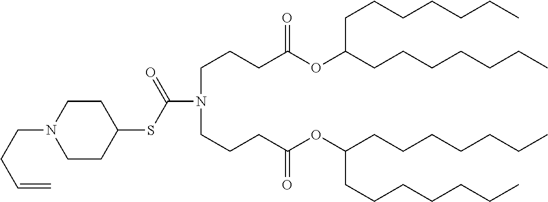 [SMILES: C=CCCN1CCC(SC(=O)N(CCCC(=O)OC(CCCCCCC)CCCCCCC)CCCC(=O)OC(CCCCCCC)CCCCCCC)CC1] |
| CAT28 | 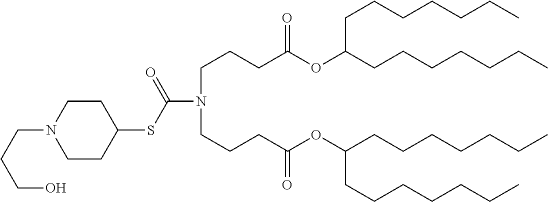 [SMILES: CCCCCCCC(CCCCCCC)OC(=O)CCCN(CCCC(=O)OC(CCCCCCC)CCCCCCC)C(=O)SC1CCN(CCCO)CC1] |
| CAT29 | 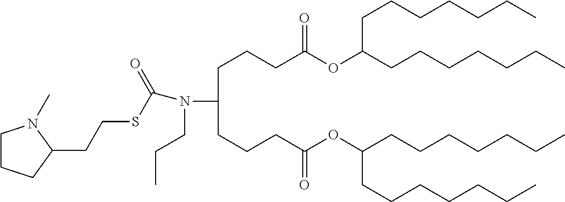 [SMILES: CCCCCCCC(CCCCCCC)OC(=O)CCCC(CCCC(=O)OC(CCCCCCC)CCCCCCC)N(CCC)C(=O)SCCC1CCCN1C] |
| CAT30 | 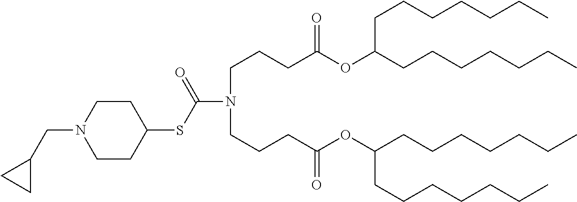 [SMILES: CCCCCCCC(CCCCCCC)OC(=O)CCCN(CCCC(=O)OC(CCCCCCC)CCCCCCC)C(=O)SC1CCN(CC2CC2)CC1] |
| CAT31 | 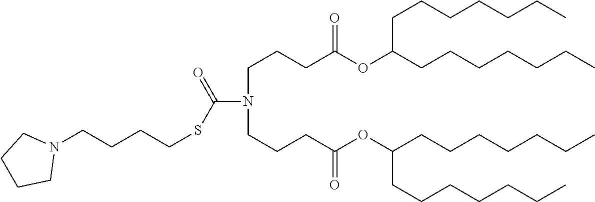 [SMILES: CCCCCCCC(CCCCCCC)OC(=O)CCCN(CCCC(=O)OC(CCCCCCC)CCCCCCC)C(=O)SCCCCN1CCCC1] |
| CAT32 | 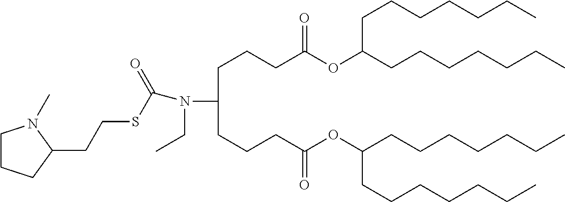 [SMILES: CCCCCCCC(CCCCCCC)OC(=O)CCCC(CCCC(=O)OC(CCCCCCC)CCCCCCC)N(CC)C(=O)SCCC1CCCN1C] |
| CAT33 | 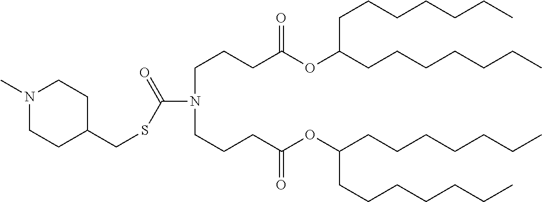 [SMILES: CCCCCCCC(CCCCCCC)OC(=O)CCCN(CCCC(=O)OC(CCCCCCC)CCCCCCC)C(=O)SCC1CCN(C)CC1] |
| CAT34 | 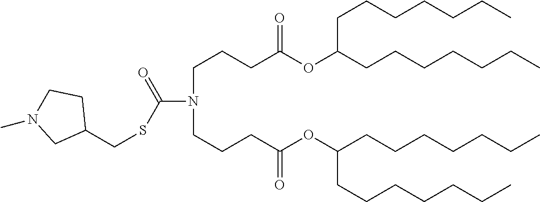 [SMILES: CCCCCCCC(CCCCCCC)OC(=O)CCCN(CCCC(=O)OC(CCCCCCC)CCCCCCC)C(=O)SCC1CCN(C)C1] |
| CAT35 | 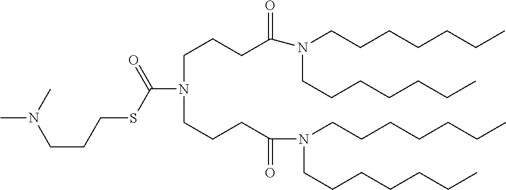 [SMILES: CCCCCCCN(CCCCCCC)C(=O)CCCN(CCCC(=O)N(CCCCCCC)CCCCCCC)C(=O)SCCCN(C)C] |

### Compounds of Formula (A)

In various embodiments, provided herein are compounds of Formula (A):

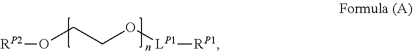

or a pharmaceutically acceptable salt thereof, wherein:
n is an integer between 10 to 200, inclusive of all endpoints;
L^{P1} is —[(CH₂)₀₋₃—C(O)O]₁₋₃—, —(CH₂)₀₋₃—C(O)O—(CH₂)₁₋₃—OC(O)—, or —C(O)N(H)—;
R¹ is C₅-C₂₅ alkyl or C₅-C₂₅ alkenyl; and
R^{P2} is hydrogen or —CH₃.

In some embodiments, Formula (A) is not HO—(CH₂CH₂₀)ₙ—C(O)N(H)—(CH₂)₁₇CH₃.

In some embodiments, L^{P1} is —CH₂C(O)O—, —CH₂CH₂C(O)O—, —CH₂C(O)OCH₂C(O)O—, —CH₂C(O)OCH₂CH₂₀C(O)—, or —C(O)N(H)—.

In some embodiments, the PEG-lipid is a compound of Formula (A-a), Formula (A-b), Formula (A-c), Formula (A-d), or Formula (A-e):

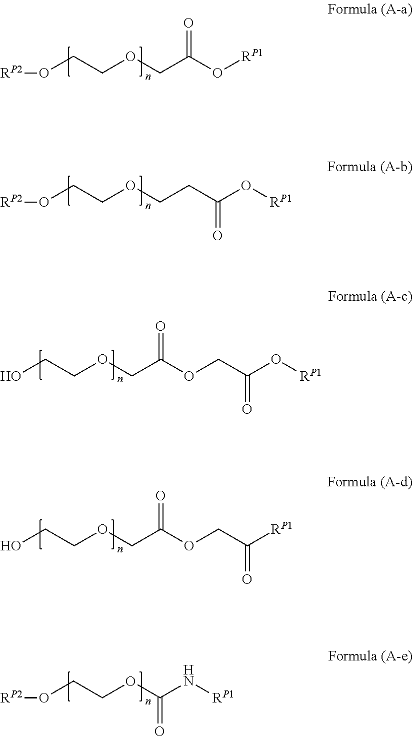

or a pharmaceutically acceptable salt thereof.

In some embodiments, R¹ is C₆-C₂₄, C₁₀-C₂₀, C₁₀-C₁₈, C₁₀-C₁₆, C₁₀-C₁₄, C₁₀-C₁₂, C₁₂-C₂₀, C₁₂-C₁₈, C₁₂-C₁₆, C₁₂-C₁₄, C₁₄-C₂₀, C₁₄-C₁₈, C₁₄-C₁₆, C₁₆-C₂₀, C₁₆-C₁₈, or C₁₈-C₂₀ alkyl. In some embodiments, R¹ is C₁₄-Cis alkyl. In some embodiments, R¹ is C₁₄-C₁₆ alkyl. In some embodiments, R¹ is C₁₅-C₁₇ alkyl. In some embodiments, R¹ is C₁₆-Cis alkyl. In some embodiments, R¹ is C₆, C₇, C₈, C₉, C₁₀, C₁₁, C₁₂, C₁₃, C₁₄, C₁₅, C₁₆, C₁₇, Cis, C₁₉, C₂₀, C₂₁, C₂₂, C₂₃, or C₂₄ alkyl. In some embodiments, R¹ is C₆-C₂₄, C₁₀-C₂₀, C₁₀-Cis, C₁₀-C₁₆, C₁₀-C₁₄, C₁₀-C₁₂, C₁₂-C₂₀, C₁₂-Cis, C₁₂-C₁₆, C₁₂-C₁₄, C₁₄-C₂₀, C₁₄-Cis, C₁₄-C₁₆, C₁₆-C₂₀, C₁₆-C₁₈, or C₁₈-C₂₀ alkenyl. In some embodiments, R^{P1} is C₁₄-Cis alkenyl. In some embodiments, R^{P1} is C₁₄-16 alkenyl. In some embodiments, R¹ is C₁₅-C₁₇ alkenyl. In some embodiments, R^{P1} is C₁₆₋₁₈ alkenyl. In some embodiments, R¹ is C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, or C₂₄ alkenyl.

In some embodiments, R^{P2} is hydrogen. In some embodiments, R² is —CH₃.

In some embodiments, n is, on average, 10 to 200, 10 to 180, 10 to 160, 10 to 140, 10 to 120, 10 to 100, 10 to 80, 10 to 60, 10 to 40, 10 to 20, 20 to 200, 20 to 180, 20 to 160, 20 to 140, 20 to 120, 20 to 100, 20 to 80, 20 to 60, 20 to 40, 40 to 200, 40 to 180, 40 to 160, 40 to 140, 40 to 120, 40 to 100, 40 to 80, 40 to 60, 60 to 200, 60 to 180, 60 to 160, 60 to 140, 60 to 120, 60 to 100, 60 to 80, 80 to 200, 80 to 180, 80 to 160, 80 to 140, 80 to 120, 80 to 100, 100 to 200, 100 to 180, 100 to 160, 100 to 140, 100 to 120, 120 to200, 120 to 180, 120 to 160, 120 to 140, 140 to 200, 140 to 180, 140 to 160, 160 to 200, 160 to 180, or 180 to 200. In some embodiments, n is, on average, 10, 20, 30, 40, 50, 60, 70, 80, 90, 100, 110, 120, 130, 140, 150, 160, 170, 180, 190, or 200. In some embodiments, n is on average about 20. In some embodiments, n is on average about 40. In some embodiments, n is on average about 45. In some embodiments, n is on average about 50. In some embodiments, n is on average about 68. In some embodiments, n is on average about 75. In some embodiments, n is on average about 100.

In some embodiments, a compound of Formula (A) is a compound selected from the group consisting of:
HO—(CH₂CH₂₀)ₙ—CH₂C(O)O—(CH₂)₁₇CH₃, n is on average about 45;
H₃CO—(CH₂CH₂₀)ₙ—CH₂C(O)O—(CH₂)₁₇CH₃, n is on average about 45;
HO—(CH₂CH₂O)ₙ—CH₂C(O)O—(CH₂)₁₅CH₃, n is on average about 45;
HO—(CH₂CH₂O)ₙ—CH₂C(O)O—(CH₂)₁₃CH₃, n is on average about 45; and
HO—(CH₂CH₂O)ₙ—C(O)N(H)—(CH₂)₁₇CH₃, n is on average about 45; or a pharmaceutically acceptable salt thereof.

### Alternative Embodiments

In an alternative embodiment, compounds described herein may also comprise one or more isotopic substitutions. For example, hydrogen may be ²H (D or deuterium) or ³H (T or tritium); carbon may be, for example, ¹³C or ¹⁴C; oxygen may be, for example, 180; nitrogen may be, for example, ¹⁵N, and the like. In other embodiments, a particular isotope (e.g., ³H, ¹³C ¹C, ¹⁸O, or ¹⁵N) can represent at least 1%, at least 5%, at least 10%, at least 15%, at least 20%, at least 25%, at least 30%, at least 35%, at least 40%, at least 45%, at least 50%, at least 60%, at least 65%, at least 70%, at least 75%, at least 80%, at least 85%, at least 90%, at least 95%, at least 99%, or at least 99.9% of the total isotopic abundance of an element that occupies a specific site of the compound.

### Lipid Nanoparticles

In some embodiments, compounds of the present disclosure are used to form a nanoparticle. In some embodiments, the nanoparticle is a lipid nanoparticle (LNP). In some embodiments, an LNP comprises a PEG-lipid, an ionizable lipid, a helper lipid, and a structural lipid. In some embodiments, LNPs described herein are formulated for delivery of therapeutic agents to a subject in need thereof. In some embodiments, LNPs described herein are formulated for delivery of nucleic acid molecules to a subject in need thereof.

The formulation of lipids in an LNP significantly impacts the therapeutic use and efficacy of a particular LNP. For example, LNP formulations such as SS—OC/Cholesterol/DSPC/PEG2k-DPG typically display increased clearance rate upon repeat intravenous (IV) administration, e.g., in mice, non-human primates (NHPs), and/or humans and a much shorter circulation time in vivo post-second dose than post-first dose. The shortened circulation time can negatively impact the delivery efficiency of the LNPs, likely due to less exposure of the LNPs to the target. Therefore, while such formulations may be useful in delivering agents that do not require multiple administrations, their use for delivery of agents that require subsequent administration may be constrained by this shortened circulation time.

There remains a need for LNP formulations that demonstrate tunable circulation and exposure to target cells, e.g., sustained circulation and consistent exposure, in vivo upon repeat dosing. The present disclosure provides such LNP formulations by incorporating ionizable lipid and/or PEG-lipid of the disclosure into the lipid formulation of the LNP.

### Cationic Lipid

In some embodiments, the LNP provided herein comprises one or more cationic lipids. “Cationic lipid” and “ionizable lipid” are used interchangeably herein.

Cationic lipids refer to any of a number of lipid species that carry a net positive charge at a selected pH, such as physiological pH. Such lipids include, but are not limited to 1,2-DiLinoleyloxy-N,N-dimethylaminopropane (DLinDMA), 1,2-Dilinolenyloxy-N,N-dimethylaminopropane (DLenDMA), dioctadecyldimethylammonium (DODMA), distearyldimethylammonium (DSDMA), N,N-dioleyl-N,N-dimethylammonium chloride (DODAC); N-(2,3-dioleyloxy)propyl)-N,N,N-trimethylammonium chloride (DOTMA); N,N-distearyl-N,N-dimethylammonium bromide (DDAB); N-(2,3-dioleoyloxy)propyl)-N,N,N-trimethylammonium chloride (DOTAP); 3-(N—(N′,N′-dimethylaminoethane)—carbamoyl)cholesterol (DC-Chol), and N-(1,2-dimyristyloxyprop-3-yl)-N,N-dimethyl-N-hydroxyethyl ammonium bromide (DMRIE). For example, cationic lipids that have a positive charge at below physiological pH include, but are not limited to, DODAP, DODMA, and DMDMA. In some embodiments, the cationic lipids comprise C18 alkyl chains, ether linkages between the head group and alkyl chains, and 0 to 3 double bonds. Such lipids include, e.g., DSDMA, DLinDMA, DLenDMA, and DODMA.

In some embodiments, the cationic lipids comprise a protonatable tertiary amine head group. Such lipids are referred to herein as ionizable lipids. Ionizable lipids refer to lipid species comprising an ionizable amine head group and typically comprising a pKa of less than about 7. Therefore, in environments with an acidic pH, the ionizable amine head group is protonated such that the ionizable lipid preferentially interacts with negatively charged molecules (e.g., nucleic acids such as the recombinant polynucleotides described herein) thus facilitating nanoparticle assembly and encapsulation. Therefore, in some embodiments, ionizable lipids can increase the loading of nucleic acids into lipid nanoparticles. In environments where the pH is greater than about 7 (e.g., physiologic pH of ˜7.4), the ionizable lipid comprises a neutral charge. When particles comprising ionizable lipids are taken up into the low pH environment of an endosome (e.g., pH<7), the ionizable lipid is again protonated and associates with the anionic endosomal membranes, promoting release of the contents encapsulated by the particle. In some embodiments, the LNP comprises an ionizable lipid, e.g., a 7.SS-cleavable and pH-responsive Lipid Like Material (such as the COATSOME@ SS-Series).

In some embodiments, the ionizable lipid is selected from DLinDMA, DLin-KC2-DMA, DLin-MC3-DMA (MC3), COATSOME® SS-LC (former name: SS-18/4PE-13), COATSOME® SS-EC (former name: SS-33/4PE-15), COATSOME® SS—OC, COATSOME® SS—OP, Di((Z)-non-2-en-1-yl)9-((4-dimethylamino)butanoyl)oxy) heptadecanedioate (L-319), N-(2,3-dioleoyloxy)propyl)-N,N,N-trimethylammonium chloride (DOTAP), or a mixture thereof.

In some embodiments the cationic lipid of the LNP is a compound of Formula (I):

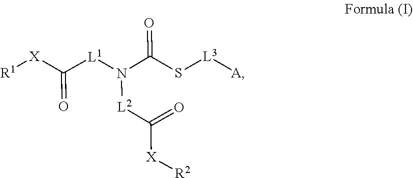

or a pharmaceutically acceptable salt or solvate thereof, wherein the variables are defined herein.

In some embodiments, cationic lipid of the disclosure is a compound selected from Table 1 or a pharmaceutically acceptable salt thereof.

In some embodiments, the cationic lipid of the LNP is a compound of Formula (II-1):

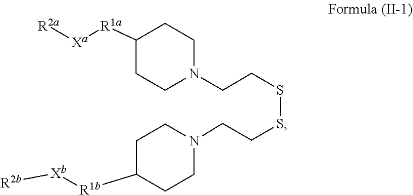

or a pharmaceutically acceptable salt or solvate thereof, wherein: R^{1a} and R^{1b} are each independently C₁-C₈ aliphatic or —O(C₁-C₈ aliphatic)-, wherein the O atom, when present, is bonded to the piperidine ring; X^{a} and X^{b} are each independently —C(O)O—*, —OC(O)—*, —C(O)N(Rₓ¹)—*, —N(Rₓ¹)C(O)—*, —O(C═O)N(Rₓ¹)—*, —N(Rₓ)(C═O)O—*, or —O—, wherein -* indicates the attachment point to R^{2a} or R^{2b}, respectively and wherein each occurrence of Rₓ¹ is independently selected from hydrogen and optionally substituted C₁-C₄ alkyl; and R^{2a} and R^{2b} are each independently a sterol residue, a liposoluble vitamin residue, or an C₁₃-C₂₃ aliphatic.

In some embodiments, the cationic lipid of the LNP is a compound of Formula (II-2):

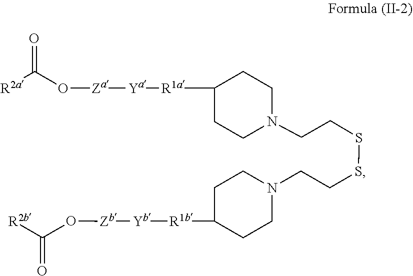

or a pharmaceutically acceptable salt or solvate thereof, wherein:
R^{1a′} and R^{1b′} are each independently C₁-C₈ alkylene or —O(C₁-C₈ alkylene), wherein the 0 atom, when present, is bonded to the piperidine ring;
Y^{a′} and Y^{b′} are each independently —C(O)O—*, —OC(O)—*, —C(O)N(Rₓ¹)—*, —N(Rₓ¹)C(O)—*, —O(C═O)N(Rₓ¹)—*, —N(Rₓ¹)(C═O)₀—*, —N(Rₓ¹)C(O)N(Rₓ¹)—, or —O—, wherein -* indicates the attachment point to R^{2a} or R^{2b}, and wherein each occurrence of Rₓ¹ is independently selected from hydrogen and optionally substituted C₁-C₄ alkyl;
Z^{a′} and Z^{b′} are each independently optionally substituted arylene-C₀-C₈ alkylene or optionally substituted arylene-C₀-C₈ heteroalkylene, wherein the alkylene or heteroalkylene group is bonded to Y^{a′} and Y^{b′}, respectively;
R^{2a′} and R^{2b′} are each independently a sterol residue, a liposoluble vitamin residue, or an C₁₂-C₂₂ aliphatic.

In some embodiments, the cationic lipid of the LNP is a compound of Formula (II-1a) (COATSOME® SS—OC) or Formula (II-2a) (COATSOME® SS—OP):

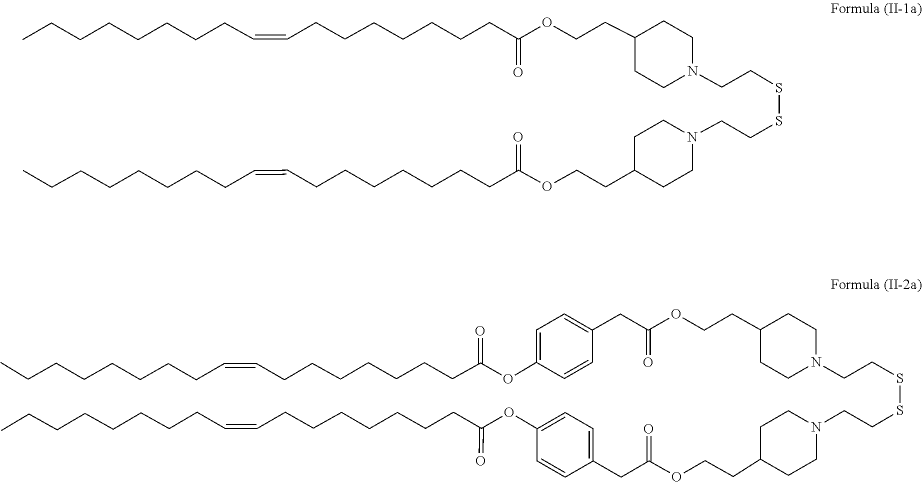 [SMILES: CCCCCCCC/C=C\CCCCCCCC(=O)OCCC1CCN(CCSSCCN2CCC(CCOC(=O)CCCCCCC/C=C\CCCCCCCC)CC2)CC1 ; CCCCCCCC/C=C\CCCCCCCC(=O)Oc1ccc(CC(=O)OCCC2CCN(CCSSCCN3CCC(CCOC(=O)Cc4ccc(OC(=O)CCCCCCC/C=C\CCCCCCCC)cc4)CC3)CC2)cc1]

In some embodiments, the cationic lipid of the LNP is a compound of Formula (II-1a) (COATSOME® SS—OC). COATSOME® SS—OC is also known as SS-18/4PE-16.

In some embodiments, the cationic lipid of the LNP is a compound of Formula (II-2a) (COATSOME® SS—OP).

In some embodiments, the cationic lipid of the LNP is 1,2-dioleoyl-3-trimethylammonium-propane (DOTAP).

### Helper Lipid

In some embodiments, the LNP described herein comprises one or more helper lipids. The term “helper lipid” refers to a lipid capable of increasing the delivery of the LNP to a target, e.g., into a cell. Without wishing to be bound by any particular theory, it is contemplated that a helper lipid may enhance the stability and/or membrane fusogenicity of the lipid nanoparticle. In some embodiments, the helper lipid is a phospholipid. In some embodiments, the helper lipid is a phospholipid substitute or replacement. In some embodiments the helper lipid is an alkyl resorcinol.

In some embodiments, the helper lipid is a phosphatidyl choline (PC). In some embodiments, the helper lipid is not a phosphatidyl choline (PC). In some embodiments the helper lipid is a phospholipid or a phospholipid substitute. In some embodiments, the phospholipid or phospholipid substitute can be, for example, one or more saturated or (poly)unsaturated phospholipids, or phospholipid substitutes, or a combination thereof. In general, phospholipids comprise a phosphate head group and one or more fatty acid tails. In some embodiments, a phospholipid may include one or more multiple (e.g., double or triple) bonds (i.e., one or more unsaturations). In some embodiments, the helper lipid is non-cationic.

A phosphate head group can be selected, for example, from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin.

A fatty acid tail can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid.

Phospholipids include, but are not limited to, glycerophospholipids such as phosphatidylcholines, phosphatidylethanolamines, phosphatidylserines, phosphatidylinositols, phosphatidy glycerols, and phosphatidic acids. Phospholipids also include phosphosphingolipid, such as sphingomyelin.

In some embodiments, the non-cationic helper lipid is a DSPC analog, a DSPC substitute, oleic acid, or an oleic acid analog.

In some embodiments, a non-cationic helper lipid is a non-phosphatidyl choline (PC) zwitterionic lipid, a DSPC analog, oleic acid, an oleic acid analog, or a 1,2-distearoyl-sn-glycero-3-phosphocholine (DSPC) substitute.

In some embodiments, the phospholipids may facilitate fusion to a membrane. For example, a cationic phospholipid may interact with one or more negatively charged phospholipids of a membrane (e.g., a cellular or intracellular membrane). Fusion of a phospholipid to a membrane may allow one or more elements of a lipid-containing composition to pass through the membrane permitting, e.g., delivery of the one or more elements to a cell.

In some embodiments, a phosphate head group can be selected from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin. A fatty acid tail can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid.

In some embodiments, the LNPs comprise one or more non-cationic helper lipids (e.g., neutral lipids). Exemplary neutral helper lipids include (1,2-dilauroyl-sn-glycero-3-phosphoethanolamine) (DLPE), 1,2-diphytanoyl-sn-glycero-3-phosphoethanolamine (DiPPE), 1,2-distearoyl-sn-glycero-3-phosphocholine (DSPC), 1,2-dipalmitoyl-sn-glycero-3-phosphocholine (DPPC), 1,2-dioleyl-sn-glycero-3-phosphoethanolamine (DOPE), 1,2-dipalmitoyl-sn-glycero-3-phosphoethanolamine (DPPE), 1,2-dimyristoyl-sn-glycero-3-phosphoethanolamine (DMPE), (1,2-dioleoyl-sn-glycero-3-phospho-(1′-rac-glycerol) (DOPG), 1,2-dioleoyl-sn-glycero-3-phosphocholine (DOPC), 1,2-distearoyl-sn-glycero-3-phosphoethanolamine (DSPE), ceramides, and sphingomyelins. In some embodiments, the one or more helper lipids are selected from 1,2-distearoyl-sn-glycero-3-phosphocholine (DSPC); 1,2-dilauroyl-sn-glycero-3-phosphoethanolamine (DLPE); 1,2-dioleoyl-sn-glycero-3-phosphocholine (DOPC); and 1,2-dioleoyl-sn-glycero-3-phosphoethanolamine (DOPE). In some embodiments, the help lipid of the LNPs comprises, consists essentially of, or consist of 1,2-Dilauroyl-sn-glycero-3-phosphoethanolamine (DLPE) or 1,2-Dioleoyl-sn-glycero-3-phosphoethanolamine (DOPE). In some embodiments, the LNP comprises DSPC. In some embodiments, the LNP comprises DOPC. In some embodiments, the LNP comprises DLPE. In some embodiments, the LNP comprises DOPE.

In some embodiments, the phospholipid is selected from the non-limiting group consisting of 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C₁₆ Lyso PC), 1,2-dilinolenoyl-sn-glycero-3-phosphocholine (18:3 (cis) PC), 1,2-diarachidonoyl-sn-glycero-3-phosphocholine (DAPC), 1,2-didocosahexaenoyl-sn-glycero-3-phosphocholine (22:6 (cis) PC) 1,2-diphytanoyl-sn-glycero-3-phosphoethanolamine (4ME 16.0 PE), 1,2-distearoyl-sn-glycero-3-phosphoethanolamine (DSPE), 1,2-dilinoleoyl-sn-glycero-3-phosphoethanolamine (PE(18:2/18:2), 1,2-dilinolenoyl-sn-glycero-3-phosphoethanol amine (PE 18:3 (9Z, 12Z, 15Z), 1,2-diarachidonoyl-sn-glycero-3-phosphoethanolamine (DAPE 18:3 (9Z, 12Z, 15Z), 1,2-didocosahexaenoyl-sn-glycero-3-phosphoethanolamine (22:6 (cis) PE), 1,2-dioleoyl-sn-glycero-3-phospho-rac-(1-glycerol) sodium salt (DOPG), and sphingomyelin.

In some embodiments, a helper lipid is selected from the group consisting of distearoyl-sn-glycero-phosphoethanolamine, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoylphosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), monomethyl-phosphatidylethanolamine, dimethylphosphatidylethanolamine, 18-1-trans PE, 1-stearoyl-2-oleoylphosphatidyethanolamine (SOPE), hydrogenated soy phosphatidylcholine (HSPC), egg phosphatidylcholine (EPC), dioleoylphosphatidylserine (DOPS), sphingomyelin (SM), dimyristoyl phosphatidylcholine (DMPC), dimyristoyl phosphatidylglycerol (DMPG), distearoylphosphatidylglycerol (DSPG), dierucoylphosphatidylcholine (DEPC), palmitoyloleyolphosphatidylglycerol (POPG), dielaidoyl-phosphatidylethanolamine (DEPE), lecithin, phosphatidylethanolamine, lysolecithin, lysophosphatidylethanolamine, phosphatidyl serine, phosphatidylinositol, sphingomyelin, egg sphingomyelin (ESM), cephalin, cardiolipin, phosphatidicacid, cerebrosides, dicetylphosphate, lysophosphatidylcholine, and dilinoleoylphosphatidylcholine.

In some embodiments, the helper lipid of the disclosure is DSPC.

In some embodiments, an LNP includes DSPC. In some embodiments, an LNP includes DOPE. In some embodiments, an LNP includes DMPE. In some embodiments, an LNP includes both DSPC and DOPE.

In some embodiments, a helper lipid is selected from the group consisting of DSPC, DMPE, and DOPC or combinations thereof.

In some embodiments of the disclosure, the helper lipid is

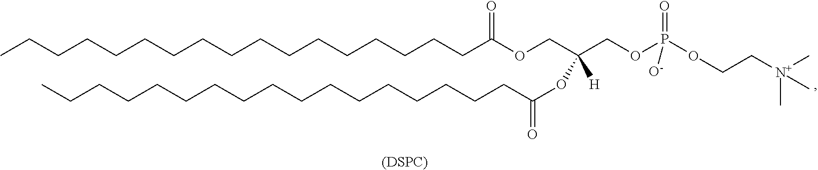

having a CAS number of 816-94-4, a linear formula of C44H88NO8P. DSPC is also known as 1,2-distearoyl-sn-glycero-3-phosphocholine.

In some embodiments, a phospholipid of the disclosure comprises a modified tail. In some embodiments, the phospholipid is DSPC (1,2-dioctadecanoyl-sn-glycero-3-phosphocholine), or analog thereof, with a modified tail. As described herein, a “modified tail” may be a tail with shorter or longer aliphatic chains, aliphatic chains with branching introduced, aliphatic chains with substituents introduced, aliphatic chains wherein one or more methylenes are replaced by cyclic or heteroatom groups, or any combination thereof.

In some embodiments, the helper lipid of the disclosure is an alternative lipid that is not a phospholipid.

In some embodiments, a phospholipid useful in the present disclosure comprises a modified tail. In some embodiments, a phospholipid useful in the present disclosure is DSPC, or analog thereof, with a modified tail. As described herein, a “modified tail” may be a tail with shorter or longer aliphatic chains, aliphatic chains with branching introduced, aliphatic chains with substituents introduced, aliphatic chains wherein one or more methylenes are replaced by cyclic or heteroatom groups, or any combination thereof.

In some embodiments, a phospholipid useful in the present disclosure comprises a modified phosphocholine moiety, wherein the alkyl chain linking the quaternary amine to the phosphoryl group is not ethylene (e.g., n is not 2).

In some embodiments, the LNP of the disclosure comprises an oleic acid or an oleic acid analog as the helper lipid. In some embodiments, an oleic acid analog comprises a modified oleic acid tail, a modified carboxylic acid moiety, or both. In some embodiments, an oleic acid analog is a compound wherein the carboxylic acid moiety of oleic acid is replaced by a different group.

In some embodiments, the LNP of the disclosure comprises a different zwitterionic group in place of a phospholipid as the helper lipid.

In some embodiments, the helper lipid of the disclosure is a naturally occurring membrane lipid. In some embodiments, the helper lipid of the disclosure is 1,2-Dipalmitoyl-sn-glycero-3-O—4′—(N,N,N-trimethyl)-homoserine (DGTS), Monogalactosyldiacylglycerol (MGDG), Digalactosyldiacylglycerol (DGDG), Sulfoquinovosyldiacylglycerol (SQDG), 1-Palmitoyl-2-cis-9,10-methylenehexadecanoyl-sn-glycero-3-phosphocholine (Cyclo PC), or a combination thereof. In some embodiments, the LNP of the disclosure comprises a combination of helper lipids. In some embodiments, the combination of helper lipids does not comprise DSPC. In some embodiments, the combination of helper lipid comprises DSPC. In some embodiments, the LNP comprising one or more naturally occurring membrane lipids (e.g., DGTS) has improved liver transfection/delivery of the payload molecule encapsulated in the LNP as compared to the LNP comprising DSPC as the only helper lipid.

In some embodiments, the helper lipid of disclosure is 5-heptadecylresorcinol or a derivative thereof.

### Structural Lipid

In some embodiments, the LNP of the disclosure comprises one or more structural lipids. Incorporation of structural lipids in the lipid nanoparticle may help mitigate aggregation of other lipids in the particle. Structural lipids may be, but are not limited to, sterols or lipids containing sterol moieties.

In some embodiments, the structural lipid of the LNP is a sterol (e.g., phytosterols or zoosterols). In some embodiments, the sterol is cholesterol, or an analog or a derivative thereof. In some embodiments, the sterol is cholesterol. In some embodiments, the sterol is cholesterol, P-sitosterol, fecosterol, ergosterol, sitosterol, campesterol, stigmasterol, brassicasterol, ergosterol, tomatidine, tomatine, ursolic acid, alpha-tocopherol, including analogs, salts or esters thereof, alone or in combination.

In some embodiments, the structural lipid of the LNP is a cholesterol, a corticosteroid (such as, for example, prednisolone, dexamethasone, prednisone, and hydrocortisone), or a combination thereof.

In some embodiments, the structural lipid of the LNP is a pytosterol. In some embodiments, the phytosterol is a sitosterol, a stigmasterol, a campesterol, a sitostanol, a campestanol, a brassicasterol, a fucosterol, beta-sitosterol, stigmastanol, beta-sitostanol, ergosterol, lupeol, cycloartenol, A5-avenaserol, A7-avenaserol or a A7-stigmasterol, including analogs, salts or esters thereof, alone or in combination.

In some embodiments, the LNP comprises one or more phytosterols. In some embodiments, the phytosterol component of the LNP is a single phytosterol. In some embodiments, the phytosterol component of the LNP of the disclosure is a mixture of different phytosterols (e.g. 2, 3, 4, 5 or 6 different phytosterols). In some embodiments, the phytosterol component of the LNP of the disclosure is a blend of one or more phytosterols and one or more zoosterols, such as a blend of a phytosterol (e.g., a sitosterol, such as beta-sitosterol) and cholesterol.

In some embodiments of the disclosure, the structural lipid of the LNP is cholesterol:

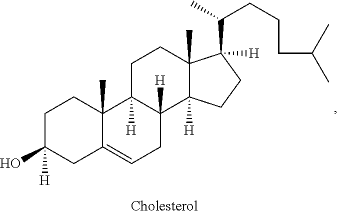

having a CAS number of 57-88-5, a linear formula of C₂₇H460.

### PEG-Lipid

In some embodiments, a PEG-lipid of the disclosure comprises a hydrophilic head group and a hydrophobic lipid tail. In some embodiments, the hydrophilic head group is a PEG moiety. In some embodiments, PEG-lipid of the disclosure comprises a mono lipid tail. In some embodiments, PEG-lipid of the disclosure comprises a mono alkyl lipid tail, a mono alkenyl lipid tail, a mono alkynyl lipid tail, or a mono acyl lipid tail. In some embodiments, the mono lipid tail comprises an ether group, a carbonyl group, or an ester group. In some embodiments, the PEG-lipid of the disclosure may contain a polyoxyethylene alkyl ether, a polyoxyethylene alkenyl ether, or a polyoxyethylene alkynyl ether (such molecules are also known as BRIJ™ molecules). In some embodiments, the PEG-lipid of the disclosure may contain a polyoxyethylene alkyl ester, a polyoxyethylene alkenyl ester, or a polyoxyethylene alkynyl ester (such molecules are also known as MYRJ™ molecules).

In some embodiments, a PEG-lipid may contain di-acyl lipid tails.

In some embodiments, the PEG-lipid is a compound of Formula (A)

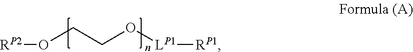

or a pharmaceutically acceptable salt or solvate thereof, wherein the variables are defined herein.

In some embodiments, the PEG-lipid is a compound of Formula (A′):

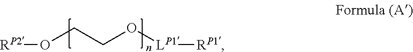

or a pharmaceutically acceptable salt thereof, wherein:
n is an integer between 10 to 200, inclusive of all endpoints;
L^{P1′} is a bond, —C(O)—, —[(CH₂)₀₋₃—C(O)O]₁₋₃—, —(CH₂)₀₋₃—C(O)O—(CH₂)₁-3-OC(O)—, or —C(O)N(H)—;
R^{P1′} is C₅-C₂₅ alkyl or C₅-C₂₅ alkenyl; and
R^{P2′} is hydrogen or —CH₃.

In some embodiments, L^{P1′} is a bond, —C(O)—, —CH₂C(O)O—, —CH₂CH₂C(O)O—, —CH₂C(O)OCH₂C(O)O—, —CH₂C(O)OCH₂CH₂OC(O)—, or —C(O)N(H)—. In some embodiments, R^{1′} is R^{P1}. In some embodiments, R^{P2′} is R^{P2}.

In some embodiments, the PEG-lipid is a compound of Formula (A″):

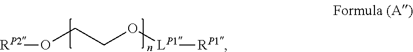

or a pharmaceutically acceptable salt thereof, wherein:
n is an integer between 10 to 200, inclusive of all endpoints;
L^{P1′} is a bond, —[(CH₂)₀₋₃—C(O)O]₁₋₃—, —(CH₂)₀₋₃—C(O)O—(CH₂)₁₋₃—OC(O)—, or —C(O)N(H)—;
R^{P1′} is C₅-C₂₅ alkyl or C₅-C₂₅ alkenyl; and
R^{P2′} is hydrogen or —CH₃.

In some embodiments, L^{P1′} is a bond, —CH₂C(O)O—, —CH₂CH₂C(O)O—, —CH₂C(O)OCH₂C(O)O—, —CH₂C(O)OCH₂CH₂OC(O)—, or —C(O)N(H)—.

In some embodiments, the PEG-lipid is a compound of Formula (A″-a), Formula (A″-b), Formula (A″-c), Formula (A″-cd), Formula (A″-e), or Formula (A″-f):

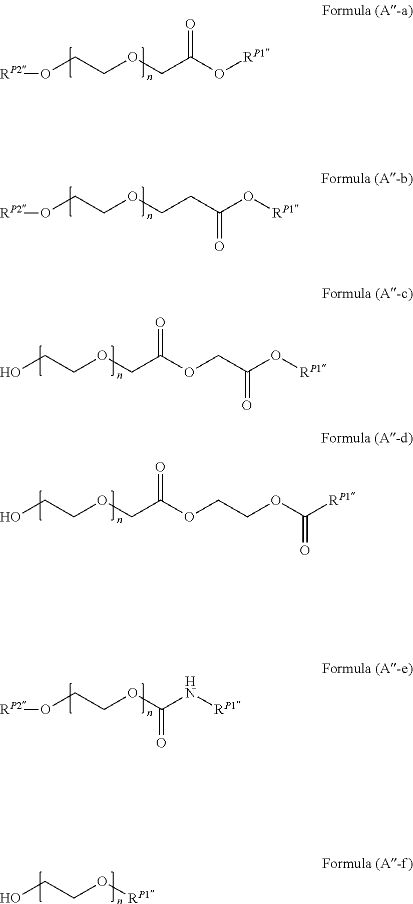

or a pharmaceutically acceptable salt thereof.

In some embodiments, R^{P1′} is R¹. In some embodiments, R^{P2′} is R^{P2}.

In some embodiments, the PEG-lipid is a compound of Formula (A″-f1):

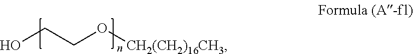

or a pharmaceutically acceptable salt thereof.

In some embodiments, the PEG-lipid is a compound of Formula (A″-f2):

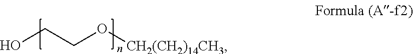

or a pharmaceutically acceptable salt thereof.

In some embodiments, the PEG-lipid is a compound of Formula (A″-f3):

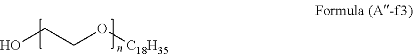

or a pharmaceutically acceptable salt thereof.

In some embodiments, a PEG-lipid of the disclosure is a compound of Formula (B):

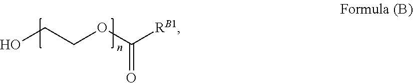

or a pharmaceutically acceptable salt thereof, wherein:
n is an integer between 10 to 200, inclusive of all endpoints; and
R^{B1} is C₅-C₂₅ alkyl or C₅-C₂₅ alkenyl.

In some embodiments, R^{B1} is R^{P1}.

In some embodiments, the PEG-lipid is a compound of Formula (B-a):

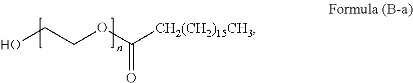

or a pharmaceutically acceptable salt thereof.

In some embodiments, the PEG-lipid is a compound of Formula (B-b):

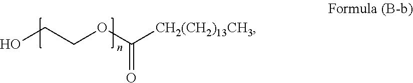

or a pharmaceutically acceptable salt thereof.

In some embodiments, n is, on average, 10 to 200, 10 to 180, 10 to 160, 10 to 140, 10 to 120, 10 to 100, 10 to 80, 10 to 60, 10 to 40, 10 to 20, 20 to 200, 20 to 180, 20 to 160, 20 to 140, 20 to 120, 20 to 100, 20 to 80, 20 to 60, 20 to 40, 40 to 200, 40 to 180, 40 to 160, 40 to 140, 40 to 120, 40 to 100, 40 to 80, 40 to 60, 60 to 200, 60 to 180, 60 to 160, 60 to 140, 60 to 120, 60 to 100, 60 to 80, 80 to 200, 80 to 180, 80 to 160, 80 to 140, 80 to 120, 80 to 100, 100 to 200, 100 to 180, 100 to 160, 100 to 140, 100 to 120, 120 to200, 120 to 180, 120 to 160, 120 to 140, 140 to 200, 140 to 180, 140 to 160, 160 to 200, 160 to 180, or 180 to 200. In some embodiments, n is, on average, 10, 20, 30, 40, 50, 60, 70, 80, 90, 100, 110, 120, 130, 140, 150, 160, 170, 180, 190, or 200. In some embodiments, n is on average about 20. In some embodiments, n is on average about 40. In some embodiments, n is on average about 45. In some embodiments, n is on average about 50. In some embodiments, n is on average about 68. In some embodiments, n is on average about 75. In some embodiments, n is on average about 100.

In some embodiments, the PEG-lipid comprises a PEG moiety having an average molecular weight of about 500 to about 10,000 daltons. In some embodiments, the PEG-lipid comprises a PEG moiety having an average molecular weight of about 500 to about 5,000 daltons, about 500 to about 4,000 daltons, about 500 to about 3,000 daltons, about 500 to about 2,000 daltons, about 500 to about 1,000 daltons, about 500 to about 800 daltons, about 500 to about 600 daltons, about 600 to about 5,000 daltons, about 600 to about 4,000 daltons, about 600 to about 3,000 daltons, about 600 to about 2,000 daltons, about 600 to about 1,000 daltons, about 600 to about 800 daltons, about 800 to about 5,000 daltons, about 800 to about 4,000 daltons, about 800 to about 3,000 daltons, about 800 to about 2,000 daltons, about 800 to about 1,000 daltons, about 1,000 to about 5,000 daltons, about 1,000 to about 4,000 daltons, about 1,000 to about 3,000 daltons, about 1,000 to about 2,000 daltons, about 2,000 to about 5,000 daltons, about 2,000 to about 4,000 daltons, about 2,000 to about 3,000 daltons, about 3,000 to about 5,000 daltons, about 3,000 to about 4,000 daltons, about 5,000 to about 10,000 daltons, about 5,000 to about 7,500 daltons, or about 7,500 to about 10,000 daltons. In some embodiments, the PEG moiety of the PEG-lipid has an average molecular weight of about 1,500 to about 2,500 daltons. In some embodiments, the PEG moiety of the PEG-lipid has an average molecular weight of about 1,000 to about 5,000 daltons. In some embodiments, the PEG-lipid comprises a PEG moiety having an average molecular weight of about 500, about 600, about 800, about 1,000, about 1,500, about 2,000, about, 2500, about 3,000, about 3,500, about 4,000, about 4,500, about 5,000, about 6,000, about 7,000, about 8,000, about 9,000, or about 10,000 daltons. In some embodiments, the PEG-lipid comprises a PEG moiety having an average molecular weight of at least 500, at least 1,000, at least 1,500, at least 2,000, at least 2,500, at least 3,000, at least 3,500, at least 4,000, at least 4,500, at least 5,000, at least 6,000, at least 7,000, at least 8,000, at least 9,000, or at least 10,000 daltons. In some embodiments, the PEG-lipid comprises a PEG moiety having an average molecular weight of no more than 500, no more than 1,000, no more than 1,500, no more than 2,000, no more than 2,500, no more than 3,000, no more than 3,500, no more than 4,000, no more than 4,500, no more than 5,000, no more than 6,000, no more than 7,000, no more than 8,000, no more than 9,000, or no more than 10,000 daltons. All values are inclusive of all endpoints.

In some embodiments, the PEG-lipid is polyoxyethylene (100) stearyl ether, polyoxyethylene (20) cetyl ether, polyoxyethylene (20) oleyl ether, polyoxyethylene (20) stearyl ether, or a mixture thereof. In some embodiments, the PEG-lipid is polyoxyethylene (100) stearate, polyoxyethylene (50) stearate, polyoxyethylene (40) stearate, polyoxyethylene palmitate, or a mixture thereof.

In some embodiments of the disclosure, the PEG-lipid is

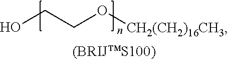

having a CAS number of 9005-00, a linear formula of C₁₈H₃₇(OCH₂CH₂)ₙOH wherein n is 100. BRIJ™ S100 is also known, generically, as polyoxyethylene (100) stearyl ether. Accordingly, in some embodiments, the PEG-lipid is HO-PEG100-CH₂(CH₂)₁₆CH₃.

In some embodiments of the disclosure, the PEG-lipid is

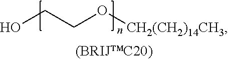

having a CAS number of 9004-95-9, a linear formula of C₁₆H₃₃(OCH₂CH₂)ₙOH wherein n is 20. BRIJ™ C₂₀ is also known as BRIJ™ 58, and, generically, as polyethylene glycol hexadecyl ether, polyoxyethylene (20) cetyl ether. Accordingly, in some embodiments, the PEG-lipid is HO-PEG20-CH₂(CH₂)₁₄CH₃.

In some embodiments of the disclosure, the PEG-lipid is

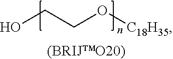

having a CAS number of 9004-98-2, a linear formula of C₁₈H₃₅(OCH₂CH₂)ₙOH wherein n is 20. BRIJ™ 020 is also known, generically, as polyoxyethylene (20) oleyl ether. Accordingly, in some embodiments, the PEG-lipid is HO-PEG20-C₁₈H₃₅.

In some embodiments of the disclosure, the PEG-lipid is

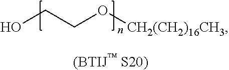

having a CAS number of 9005-00-9, a linear formula of C₁₈H₃₇(OCH₂CH₂)ₙOH wherein n is 20. BRIJ™ S20 is also known, generically, as polyethylene glycol octadecyl ether or polyoxyethylene (20) stearyl ether. Accordingly, in some embodiments, the PEG-lipid is HO-PEG20-CH₂(CH₂)₁₆CH₃.

In some embodiments of the disclosure, the PEG-lipid is

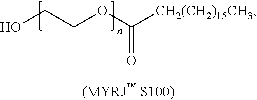

having a CAS number of 9004-99-3, a linear formula of C₁₇H₃₅C(O)(OCH₂CH₂)ₙOH wherein n is 100. MYRJ™ 5100 is also known, generically, as polyoxyethylene (100) stearate. Accordingly, in some embodiments, the PEG-lipid is HO—PEG100-CH₂(CH₂)₁₅CH₃.

In some embodiments of the disclosure, the PEG-lipid is

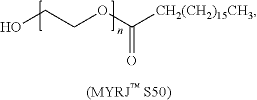

having a CAS number of 9004-99-3, a linear formula of C₁₇H₃₅C(O)(OCH₂CH₂)ₙOH wherein n is 50. MYRJ™ S50 is also known, generically, as polyoxyethylene (50) stearate. Accordingly, in some embodiments, the PEG-lipid is HO-PEG50-CH₂(CH₂)₁₅CH₃.

In some embodiments of the disclosure, the PEG-lipid is 0

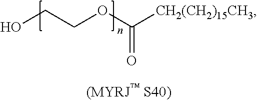

having a CAS number of 9004-99-3, a linear formula of C₁₇H35C(O)(OCH₂CH₂)ₙOH wherein n is 40. MYRJ™ S40 is also known, generically, as polyoxyethylene (40) stearate. Accordingly, in some embodiments, the PEG-lipid is HO—PEG40-CH₂(CH₂)₁₅CH₃.

In some embodiments of the disclosure, the PEG-lipid is

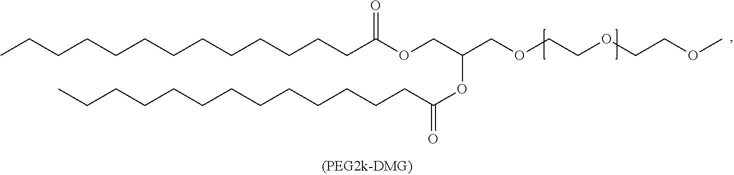

having a CAS number of 1607430-62-04, a linear formula of C122H242050. PEG2k-DMG is also known as 1,2-dimyristoyl-rac-glycero-3-methoxypolyethylene glycol-2000.

In some embodiments of the disclosure, the PEG-lipid is:

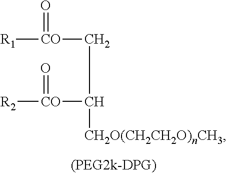

having an alkyl composition of R₁COO═C16:0, R₂COO═C16:0. PEG2k-DPG is also known, generically, as 1,2-Dipalmitoyl-rac-glycero-3-methylpolyoxyethylene.

In some embodiments of the disclosure, the PEG-lipid may be PEG-dilauroylglycerol, PEG-dimyristoylglycerol (PEG-DMG), PEG-dipalmitoylglycerol, PEG-distearoylglycerol (PEG-DSPE), PEG-dilaurylglycamide, PEG-dimyristylglycamide, PEG-dipalmitoylglycamide, PEG-distearoylglycamide, PEG-cholesterol (1-[8′-(Cholest-5-en-3[beta]-oxy)carboxamido-3′,6′-dioxaoctanyl]carbamoyl-[omega]-methyl-poly(ethylene glycol), PEG-DMB (3,4-ditetradecoxylbenzyl-[omega]-methyl-poly(ethylene glycol)ether), 1,2-dimyristoyl-sn-glycero-3-phosphoethanolamine-N-[methoxy(polyethylene glycol)-2000](PEG2k-DMG), 1,2-distearoyl-sn-glycero-3-phosphoethanolamine-N-[methoxy(polyethylene glycol)-2000] (PEG2k-DSPE), 1,2-distearoyl-snglycerol, methoxypolyethylene glycol (PEG2k-DSG), poly(ethylene glycol)-2000-dimethacrylate (PEG2k-DMA), or 1,2-distearyloxypropyl-3-amine-N-[methoxy(polyethylene glycol)-2000] (PEG2k-DSA). In some embodiments, the PEG-lipid may be PEG2k-DMG. In some embodiments, the PEG-lipid may be PEG2k-DSG. In other embodiments, the PEG-lipid may be PEG2k-DSPE. In some embodiments, the PEG-lipid may be PEG2k-DMA. In yet other embodiments, the PEG-lipid may be PEG2k-C-DMA. In some embodiments, the PEG-lipid may be PEG2k-DSA. In other embodiments, the PEG-lipid may be PEG2k-C11. In some embodiments, the PEG-lipid may be PEG2k-C14. In some embodiments, the PEG-lipid may be PEG2k-C16. In some embodiments, the PEG-lipid may be PEG2k-C18.

In some embodiments, a PEG-lipid having single lipid tail of the disclosure (e.g., PEG-lipid of Formula (A), (A′), (A″), or (B)) may reduce accelerated blood clearance (ABC) upon administration and/or repeat administration of an LNP composition of the disclosure. In some embodiments, a PEG-lipid having single lipid tail of the disclosure may reduce or deplete PEG-specific antibodies (e.g., anti-PEG IgM) generated by a subject's immune system upon administration and/or repeat administration of an LNP composition of the disclosure.

### Lipid Molar Ratio in the LNP Composition

In some embodiments, the LNP of the disclosure comprises between 40 mol % and 70 mol % of the cationic lipid, up to 50 mol % of the helper lipid, between 10 mol % and 50 mol % of the structural lipid, and between 0.001 mol % and 5 mol % of the PEG-lipid, inclusive of all endpoints. In some embodiments, the total mol % of the cationic lipid, the helper lipid, the structural lipid and the PEG-lipid is 100%.

In some embodiments, the mol % of the cationic lipid in the LNP is 40-70 mol %, 40-55 mol %, 40-50 mol %, 40-45 mol %, 44-54 mol %, 45-60 mol %, 45-55 mol %, 45-50 mol %, 50-60 mol %, 49-64 mol %, 50-55 mol %, or 55-60 mol %. In some embodiments, the mol % of the cationic lipid in the LNP is 44-54 mol %. In some embodiments, the mol % of the cationic lipid in the LNP is 40, 41, 42, 43, 44, 45, 46, 47, 48, 49, 50, 51, 52, 53, 54, 55, 56, 57, 58, 59, or 60 mol %. In some embodiments, the mol % of the cationic lipid in the LNP is about 40, about 41, about 42, about 43, about 44, about 45, about 46, about 47, about 48, about 49, about 50, about 51, about 52, about 53, about 54, about 55, about 56, about 57, about 58, about 59, or about 60 mol %. All values are inclusive of all endpoints.

In some embodiments, the mol % of the structural lipid in the LNP is 10-60 mol %, 10-30 mol %, 15-35 mol %, 20-40 mol %, 20-45 mol %, 25-33 mol %, 24-32 mol %, 25-45 mol %, 30-50 mol %, 35-43 mol %, 35-55 mol %, or 40-60 mol %. In some embodiments, the mol % of the structural lipid in the LNP is 20-45 mol %. In some embodiments, the mol % of the structural lipid in the LNP is 24-32 mol %. In some embodiments, the mol % of the structural lipid in the LNP is 25-33 mol %. In some embodiments, the mol % of the structural lipid in the LNP is 22-28 mol %. In some embodiments, the mol % of the structural lipid in the LNP is 35-45 mol %. In some embodiments, the mol % of the structural lipid in the LNP is 35-43 mol %. In some embodiments, the mol % of the structural lipid in the LNP is 10-60 mol %. In some embodiments, the mol % of the structural lipid in the LNP is 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 mol %. In some embodiments, the mol % of the structural lipid in the LNP is about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, or about 60 mol %. In some embodiments, the structural lipid is cholesterol. All values are inclusive of all endpoints.

In some embodiments, the mol % of the helper lipid in the LNP is 1-50 mol %. In some embodiments, the mol % of the helper lipid in the LNP is up to 29 mol %. In some embodiments, the mol % of the helper lipid in the LNP is 1-10 mol %, 5-9 mol %, 5-15 mol %, 8-14 mol %, 18-22%, 19-25 mol %, 10-20 mol %, 10-25 mol %, 15-25 mol %, 20-30 mol %, 25-35 mol %, 30-40 mol %, or 35-50 mol %. In some embodiments, the mol % of the helper lipid in the LNP is 10-25 mol %. In some embodiments, the mol % of the helper lipid in the LNP is 5-9 mol %. In some embodiments, the mol % of the helper lipid in the LNP is 8-14 mol %. In some embodiments, the mol % of the helper lipid in the LNP is 18-22 mol %. In some embodiments, the mol % of the helper lipid in the LNP is 19-25 mol %. In some embodiments, the mol % of the helper lipid in the LNP is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 mol %. In some embodiments, the mol % of the helper lipid in the LNP is about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, or about 40 mol %. In some embodiments, the helper lipid is DSPC. All values are inclusive of all endpoints.

In some embodiments, the mol % of the PEG-lipid in the LNP is greater than 0 mol % and up to 5 mol % of the total lipid present in the LNP. In some embodiments, the mol % of the PEG-lipid is 0.1 mol %, 0.2 mol %, 0.25 mol %, 0.3 mol %, 0.4 mol %, 0.5 mol %, 0.6 mol %, 0.7 mol %, 0.8 mol %, 0.9 mol %, 1.0 mol %, 1.1 mol %, 1.2 mol %, 1.3 mol %, 1.4 mol %, 1.5 mol %, 1.6 mol %, 1.7 mol %, 1.8 mol %, 1.9 mol %, 2.0 mol %, 2.1 mol %, 2.2 mol %, 2.3 mol %, 2.4 mol %, 2.5 mol %, 2.6 mol %, 2.7 mol %, 2.8 mol %, 2.9 mol %, 3.0 mol %, 3.1 mol %, 3.2 mol %, 3.3 mol %, 3.4 mol %, 3.5 mol %, 4.0 mol %, 4.5 mol %, or 5 mol % of the total lipid present in the LNP. In some embodiments, the mol % of the PEG-lipid is about 0.1 mol %, about 0.2 mol %, about 0.25 mol %, about 0.3 mol %, about 0.4 mol %, about 0.5 mol %, about 0.6 mol %, about 0.7 mol %, about 0.8 mol %, about 0.9 mol %, about 1.0 mol %, about 1.1 mol %, about 1.2 mol %, about 1.3 mol %, about 1.4 mol %, about 1.5 mol %, about 1.6 mol %, about 1.7 mol %, about 1.8 mol %, about 1.9 mol %, about 2.0 mol %, about 2.1 mol %, about 2.2 mol %, about 2.3 mol %, about 2.4 mol %, about 2.5 mol %, about 2.6 mol %, about 2.7 mol %, about 2.8 mol %, about 2.9 mol %, about 3.0 mol %, about 3.1 mol %, about 3.2 mol %, about 3.3 mol %, about 3.4 mol %, about 3.5 mol %, about 4.0 mol %, about 4.5 mol %, or about 5 mol % of the total lipid present in the LNP. In some embodiments, the mol % of the PEG-lipid is at least 0.1 mol %, at least 0.2 mol %, at least 0.25 mol %, at least 0.3 mol %, at least 0.4 mol %, at least 0.5 mol %, at least 0.6 mol %, at least 0.7 mol %, at least 0.8 mol %, at least 0.9 mol %, at least 1.0 mol %, at least 1.1 mol %, at least 1.2 mol %, at least 1.3 mol %, at least 1.4 mol %, at least 1.5 mol %, at least 1.6 mol %, at least 1.7 mol %, at least 1.8 mol %, at least 1.9 mol %, at least 2.0 mol %, at least 2.1 mol %, at least 2.2 mol %, at least 2.3 mol %, at least 2.4 mol %, at least 2.5 mol %, at least 2.6 mol %, at least 2.7 mol %, at least 2.8 mol %, at least 2.9 mol %, at least 3.0 mol %, at least 3.1 mol %, at least 3.2 mol %, at least 3.3 mol %, at least 3.4 mol %, at least 3.5 mol %, at least 4.0 mol %, at least 4.5 mol %, or at least 5 mol % of the total lipid present in the LNP. In some embodiments, the mol % of the PEG-lipid is at most 0.1 mol %, at most 0.2 mol %, at most 0.25 mol %, at most 0.3 mol %, at most 0.4 mol %, at most 0.5 mol %, at most 0.6 mol %, at most 0.7 mol %, at most 0.8 mol %, at most 0.9 mol %, at most 1.0 mol %, at most 1.1 mol %, at most 1.2 mol %, at most 1.3 mol %, at most 1.4 mol %, at most 1.5 mol %, at most 1.6 mol %, at most 1.7 mol %, at most 1.8 mol %, at most 1.9 mol %, at most 2.0 mol %, at most 2.1 mol %, at most 2.2 mol %, at most 2.3 mol %, at most 2.4 mol %, at most 2.5 mol %, at most 2.6 mol %, at most 2.7 mol %, at most 2.8 mol %, at most 2.9 mol %, at most 3.0 mol %, at most 3.1 mol %, at most 3.2 mol %, at most 3.3 mol %, at most 3.4 mol %, at most 3.5 mol %, at most 4.0 mol %, at most 4.5 mol %, or at most 5 mol % of the total lipid present in the LNP. In some embodiments, the mol % of the PEG-lipid is between 0.1-4 mol % of the total lipid present in the LNP. In some embodiments, the mol % of the PEG-lipid is between 0.1-2 mol % of the total lipid present in the LNP. In some embodiments, the mol % of the PEG-lipid is between 0.2-0.8 mol %, 0.4-0.6 mol %, 0.7-1.3 mol %, 1.2-1.8 mol %, or 1-3.5 mol % of the total lipid present in the LNP. In some embodiments, the mol % of the PEG-lipid is 0.1-0.7 mol %, 0.2-0.8 mol %, 0.3-0.9 mol %, 0.4-0.8 mol %, 0.4-0.6 mol %, 0.4-1 mol %, 0.5-1.1 mol %, 0.6-1.2 mol %, 0.7-1.3 mol %, 0.8-1.4 mol %, 0.9-1.5 mol %, 1-3.5 mol % 1-1.6 mol %, 1.1-1.7 mol %, 1.2-1.8 mol %, 1.3-1.9 mol %, 1.4-2 mol %, 1.5-2.1 mol %, 1.6-2.2 mol %, 1.7-2.3 mol %, 1.8-2.4 mol %, 1.9-2.5 mol %, 2-2.6 mol %, 2.4-3.8 mol %, or 2.6-3.4 mol % of the total lipid present in the LNP. All values are inclusive of all endpoints.

In some embodiments, the LNP of the disclosure comprises 44-60 mol % of the cationic lipid, 19-25 mol % of the helper lipid, 25-33 mol % of the structural lipid, and 0.2-0.8 mol % of the PEG-lipid, inclusive of the endpoints. In some embodiments, the LNP of the disclosure comprises 44-54 mol % of the cationic lipid, 19-25 mol % of the helper lipid, 24-32 mol % of the structural lipid, and 1.2-1.8 mol % of the PEG-lipid, inclusive of the endpoints. In some embodiments, the LNP of the disclosure comprises 44-54 mol % of the cationic lipid, 8-14 mol % of the helper lipid, 35-43 mol % of the structural lipid, and 1.2-1.8 mol % of the PEG-lipid, inclusive of the endpoints. In some embodiments, the LNP of the disclosure comprises 45-55 mol % of the cationic lipid, 5-9 mol % of the helper lipid, 36-44 mol % of the structural lipid, and 2.5-3.5 mol % of the PEG-lipid, inclusive of the endpoints.

In some embodiments, the LNP of the disclosure comprises one or more of the cationic lipids of the disclosure, one or more helper lipids of the disclosure, one or more structural lipids of the disclosure, and one or more PEG-lipid of the disclosure at a mol % of total lipid (or the mol % range of total lipid) in the LNP according to Table 2 below. In some embodiments, the total mol % of these four lipid components equals 100%. In some embodiments, the total mol % of these four lipid components is less than 100%. In some embodiments, the cationic lipid is a compound of Formula (I) or a compound selected from Table 1. In some embodiments, the structural lipid is cholesterol. In some embodiments, the helper lipid is DSPC. In some embodiments, the PEG-lipid is of Formula (A), Formula (A′), or Formula (A″).

TABLE 2
Mol % of the Lipid Components in the LNP

| Cationic Lipid | Structural Lipid | Helper Lipid | PEG-lipid |
|---|---|---|---|
| (mol %) | (mol %) | (mol %) | (mol %) |
| 49 | 28.5 | 22 | 0.5 |
| 47-52 | 27-30 | 21-23 | 0.4-0.6 |
| 44-54 | 25-32 | 19-25 | 0.2-0.8 |
| 44-54 | 25-33 | 19-25 | 0.2-0.8 |
| 49 | 27.5 | 22 | 1.5 |
| 47-52 | 26-29 | 21-23 | 1.3-1.7 |
| 44-54 | 24-31 | 19-25 | 1.1-1.9 |
| 44-54 | 24-32 | 19-25 | 1.2-1.8 |
| 49 | 39.5 | 11 | 0.5 |
| 47-52 | 38-41 | 11-13 | 0.4-0.6 |
| 44-54 | 36-43 | 9-15 | 0.2-0.8 |
| 49 | 38.5 | 11 | 1.5 |
| 47-52 | 37-40 | 11-13 | 1.3-1.7 |
| 44-54 | 35-42 | 9-15 | 1.1-1.9 |
| 44-54 | 35-43 | 8-14 | 1.2-1.8 |
| 20-60 | 10-60 | ≥20 | 0.5 |
| 20-60 | 10-60 | ≥20 | 0.3-0.7 |
| 20-60 | 10-60 | ≥20 | 0.1-0.9 |
| 20-60 | 10-60 | 10 | 1.5 |
| 20-60 | 10-60 | 8-12 | 1.3-1.7 |
| 20-60 | 10-60 | 6-14 | 1.1-1.9 |
| 50 | 40 | 7 | 3 |
| 45-55 | 35-45 | 5-9 | 2.5-3.5 |
| 54.5 | 25 | 20 | 0.5 |
| 50-60 | 22-28 | 18-22 | 0.3-0.7 |
| 54.6 | 25.1 | 20.1 | 0.25 |
| 44.5 | 50 | 5 | 0.5 |
| 40 | 50 | 8.75 | 1.25 |
| 60 | 25 | 14.5 | 0.5 |
| 60 | 34.3 | 5 | 0.7 |
| 50 | 42.5 | 7 | 0.5 |
| 58 | 33.5 | 7 | 1.5 |
| 58 | 34.5 | 7 | 0.5 |
| 35-65 | 25-55 | 5-25 | 0.3-3 |
| 49 | 28.5 | 22 | 0.5 |
| 40 | 39.5 | 20 | 0.5 |
| 40 | 50 | 8.75 | 1.25 |
| 60 | 34.3 | 5 | 0.7 |
| 54.6 | 25.1 | 20.1 | 0.25 |
| 50.1 | 42.6 | 7 | 0.24 |

### Properties of LNP Composition

The disclosure provides compositions (e.g., pharmaceutical compositions) comprising a plurality of LNPs as described herein. Also provided herein are compositions comprising LNPs as described herein and encapsulate payload molecules.

In some embodiments, the LNP of the present disclosure may reduce immune response in vivo as compared to a control LNP.

In some embodiments, the control LNP is an LNP comprising a PEG-lipid that is not of Formula (A), Formula (A′), or Formula (A″). In some embodiments, the PEG-lipid of the control LNP is PEG2k-DPG. In some embodiments, the PEG-lipid of the control LNP is PEG2k-DMG. In some embodiments, the control LNP has the same molar ratio of the PEG-lipid as the LNP of the present disclosure. In some embodiments, the control LNP is identical to an LNP of the present disclosure except that the control LNP comprises a PEG-lipid that is not of Formula (A), Formula (A′), or Formula (A″) (e.g., the control LNP may comprise PEG2k-DPG or PEG2k-DMG as PEG-lipid).

In some embodiments, the control LNP is an LNP comprising a cationic lipid that is not of Formula (I). In some embodiments, the cationic lipid of the control LNP is SS—OC. In some embodiments, the control LNP has the same molar ratio of the cationic lipid as the LNP of the present disclosure. In some embodiments, the control LNP is identical to an LNP of the present disclosure except that the control LNP comprises a cationic lipid that is not of Formula (I) (e.g., the control LNP may comprise SS—OC as cationic lipid).

In some embodiments, the reduced immune response may be a reduction in accelerated blood clearance (ABC). In some embodiments, the ABC is associated with the secretion of natural IgM and/or anti-PEG IgM. The term “natural IgM,” as used herein, refers to circulating IgM in the serum that exists independent of known immune exposure (e.g., the exposure to a LNP of the disclosure). The term “reduction of ABC” refers to any reduction in ABC in comparison to a control LNP. In some embodiments, a reduction in ABC may be a reduced clearance of the LNP upon a second or subsequent dose, relative to a control LNP. In some embodiments, the reduction may be at least 10%, 15%, 20%, 25%, 30%, 35%, 40%, 45%, 50%, 55%, 60%, 65%, 70%, 75%, 80%, 85%, 90%, 95%, 98%, or 100%. In some embodiments, the reduction is about 10% to about 100%, about 10 to about 50%, about 20 to about 100%, about 20 to about 50%, about 30 to about 100%, about 30 to about 50%, about 40% to about 100%, about 40 to about 80%, about 50 to about 90%, or about 50 to about 100%. In some embodiments, a reduction in ABC may be measured by an increase in or a sustained detectable level of an encapsulated payload following a second or subsequent administration. In some embodiments, a reduction in ABC may result in an increase (e.g., a 2-fold, a 3-fold, a 4-fold, a 5-fold, or higher fold increase) in the level of the encapsulated payload relative to the level of encapsulated payload following administration of a control LNP. In some embodiments, the reduced ABC is associated with a lower serum level of anti-PEG IgM.

In some embodiments, the LNP of the present disclosure may delay clearance of the LNP and components thereof upon repeat dosing compared to a control LNP, which may be cleared prior to payload release. Accordingly, the LNP of the present disclosure may increase the delivery efficiency of the encapsulated payload (e.g., RNA) in subsequent doses.

In some embodiments, the LNPs have an average size (i.e., average outer diameter) have an average size of about 50 nm to about 150 nm. In some embodiments, the disclosure provides a therapeutic composition comprising a plurality of lipid nanoparticles, wherein the plurality of LNPs have an average size of about 60 nm to about 130 nm. In some embodiments, the disclosure provides a therapeutic composition comprising a plurality of lipid nanoparticles, wherein the plurality of LNPs have an average size of about 70 nm to about 120 nm. In some embodiments, the disclosure provides a therapeutic composition comprising a plurality of lipid nanoparticles, wherein the plurality of LNPs have an average size of about 70 nm. In some embodiments, the disclosure provides a therapeutic composition comprising a plurality of lipid nanoparticles, wherein the plurality of LNPs have an average size of about 80 nm. In some embodiments, the disclosure provides a therapeutic composition comprising a plurality of lipid nanoparticles, wherein the plurality of LNPs have an average size of about 90 nm. In some embodiments, the disclosure provides a therapeutic composition comprising a plurality of lipid nanoparticles, wherein the plurality of LNPs have an average size of about 100 nm. In some embodiments, the disclosure provides a therapeutic composition comprising a plurality of lipid nanoparticles, wherein the plurality of LNPs have an average size of about 110 nm. All values are inclusive of end points.

In some embodiments, the encapsulation efficiency of the payload molecule by the LNP is about 70%, about 75%, about 80%, about 85%, about 90%, about 91%, about 92%, about 93%, about 94%, about 95%, about 96%, about 97%, about 98%, about 99%, or 100%. In some embodiments, about 70%, about 75%, about 80%, about 90%, about 95%, about 97%, about 98%, or about 99% of the plurality of LNPs comprises an encapsulated payload molecule. In some embodiments, the encapsulation efficiency of the payload molecule by the LNP is at least 70%, at least 75%, at least 80%, at least 85%, at least 90%, at least 91%, at least 92%, at least 93%, at least 94%, at least 95%, at least 96%, at least 97%, at least 98%, at least 99%, or 100%. In some embodiments, at least 70%, at least 75%, at least 80%, at least 90%, at least 95%, at least 97%, at least 98%, or at least 99% of the plurality of LNPs comprises an encapsulated payload molecule. In some embodiments, about 70% to 100%, about 75% to 100%, about 80% to 100%, about 85% to 100%, about 90% to 100%, about 91% to 100%, about 92% to 100%, about 93% to 100%, about 94% to 100%, about 95% to 100%, about 96% to 100%, about 97% to 100%, about 98% to 100%, about 99% to 100% of the plurality of LNPs comprises an encapsulated payload molecule.

In some embodiments, the LNPs have a neutral charge (e.g., an average zeta-potential of between about 0 mV and 1 mV). In some embodiments, the LNPs have an average zeta-potential of between about 40 mV and about −40 mV. In some embodiments, the LNPs have an average zeta-potential of between about 40 mV and about 0 mV. In some embodiments, the LNPs have an average zeta-potential of between about 35 mV and about 0 mV, about 30 mV and about 0 mV, about 25 mV to about 0 mV, about 20 mV to about 0 mV, about 15 mV to about 0 mV, about 10 mV to about 0 mV, or about 5 mV to about 0 mV. In some embodiments, the LNPs have an average zeta-potential of between about 20 mV and about −40 mV. In some embodiments, the LNPs have an average zeta-potential of between about 20 mV and about −20 mV. In some embodiments, the LNPs have an average zeta-potential of between about 10 mV and about −20 mV. In some embodiments, the LNPs have an average zeta-potential of between about 10 mV and about −10 mV. In some embodiments, the LNPs have an average zeta-potential of about 10 mV, about 9 mV, about 8 mV, about 7 mV, about 6 mV, about 5 mV, about 4 mV, about 3 mV, about 2 mV, about 1 mV, about 0 mV, about −1 mV, about −2 mV, about −3 mV, about −4 mV, about −5 mV, about −6 mV, about −7 mV, about −8 mV, about −9 mV, about −9 mV or about −10 mV.

In some embodiments, the LNPs have an average zeta-potential of between about 0 mV and −20 mV. In some embodiments, the LNPs have an average zeta-potential of less than about −20 mV. For example, in some embodiments, the LNPs have an average zeta-potential of less than about less than about −30 mV, less than about 35 mV, or less than about −40 mV. In some embodiments, the LNPs have an average zeta-potential of between about −50 mV to about −20 mV, about −40 mV to about −20 mV, or about −30 mV to about −20 mV. In some embodiments, the LNPs have an average zeta-potential of about 0 mV, about −1 mV, about −2 mV, about −3 mV, about −4 mV, about −5 mV, about −6 mV, about −7 mV, about −8 mV, about −9 mV, about −10 mV, about −11 mV, about −12 mV, about −13 mV, about −14 mV, about −15 mV, about −16 mV, about −17 mV, about −18 mV, about −19 mV, about −20 mV, about −21 mV, about −22 mV, about −23 mV, about −24 mV, about −25 mV, about −26 mV, about −27 mV, about −28 mV, about −29 mV, about −30 mV, about −31 mV, about −32 mV, about −33 mV, about −34 mV, about −35 mV, about −36 mV, about −37 mV, about −38 mV, about −39 mV, or about −40 mV. In some embodiments, the LNPs have an average zeta-potential of less than about −20 mV, less than about −30 mV, less than about 35 mV, or less than about −40 mV.

In some embodiments, the LNP comprises a synthetic RNA viral genome encoding an oncolytic virus, wherein the encoded oncolytic virus is capable of reducing the size of a tumor that is remote from the site of LNP administration to a subject. For example, in some embodiments, intravenous administration of the LNPs of the disclosure results in viral replication in tumor tissue and reduction of tumor size for tumors or cancerous tissues that are remote from the site of LNP administration. Such effects enable the use of the LNP-encapsulated oncolytic viruses described herein in the treatment of tumors that are not easily accessible and therefore not suitable for intratumoral delivery of treatment.

### Payload

The LNP of the disclosure may comprise one or more payload molecules. A payload molecule may be any molecule desired to be delivered to a target cell or subject. For example, payload molecules may be nucleic acids, polypeptides, small molecules, carbohydrates, enzymes, dyes, fluorochromes, or a combination thereof.

In some embodiments, the LNP described herein may comprise one or more payloads linked to an inner and/or outer surface of the LNP. In some embodiments, the LNP described herein may comprise one or more payload molecules integrated within one or more lipid layers, a hydrophobic compartment, a hydrophilic compartment, or an encapsulated volume of the LNP. In some embodiments, the LNP described herein comprises one or more encapsulated payload molecules.

### Nucleic Acid Molecules

In some embodiments, the disclosure provides LNPs comprising a nucleic acid payload molecule. In some embodiments, the LNP fully encapsulates the nucleic acid molecule.

In some embodiments, the LNPs comprises a DNA, a RNA, a locked nucleic acid, a protein nucleic acid (PNA), a modified nucleic acid, a nucleic acid analog, a synthetic nucleic acid, or a plasmid capable of expressing a DNA or an RNA. In some embodiments, the LNP comprises an RNA. In some embodiments, the nucleic acid molecule comprises a single-stranded RNA (ssRNA), an siRNA, a microRNA, an mRNA, or a guide RNA (gRNA). In some embodiments, the nucleic acid molecule comprises a single-stranded RNA (ssRNA). In some embodiments, the nucleic acid molecule comprises a single-stranded DNA (ssDNA) or a double-stranded DNA (dsDNA). In some embodiments, the nucleic acid molecule comprises at least one modified nucleotide. In some embodiments, the nucleic acid molecule comprises at least one 2′-O-methyl (2′-OMe) nucleotide.

In some embodiments, the nucleic acid payload is a plasmid comprising a sequence encoding a replication-competent viral genome. In an aspect, the present disclosure provides a polynucleotide sequence encoding a replication-competent viral genome, wherein the polynucleotide sequence encoding the replication-competent virus is non-viral in origin, and wherein the polynucleotide is capable of producing a replication-competent virus when introduced into a cell by a non-viral delivery vehicle.

In some embodiments, the nucleic acid payload is a recombinant DNA or RNA molecule comprising a polynucleotide sequence encoding a replication-competent viral genome, wherein the polynucleotide sequence is operably linked to promoter sequence capable of binding a mammalian RNA polymerase II (Pol II) and is flanked by a 3′ ribozyme-encoding sequence and a 5′ ribozyme-encoding sequence, wherein the polynucleotide encoding the replication-competent viral genome is non-viral in origin. In some embodiments, the nucleic acid payload is capable of producing an infectious, lytic virus when introduced into a cell by a non-viral delivery vehicle.

In some embodiments, the recombinant DNA or RNA polynucleotide further comprises one or more micro RNA (miRNA) target sequence (miR-TS) cassettes inserted into the polynucleotide encoding the replication-competent viral genome, wherein the miR-TS cassette comprises one or more miRNA target sequences, and wherein expression of one or more of the corresponding miRNAs in a cell inhibits replication of the encoded virus in the cell.

In some embodiments, the nucleic acid molecule is 1,000 to 20,000 nucleotides in length. In some embodiments, the nucleic acid molecule is 1,000 to 20,000 nucleotides, 3,000 to 20,000 nucleotides, 5,000 to 20,000 nucleotides, 7,000 to 20,000 nucleotides, 10,000 to 20,000 nucleotides, 15,000 to 20,000 nucleotides, 1,000 to 15,000 nucleotides, 3,000 to 15,000 nucleotides, 5,000 to 15,000 nucleotides, 7,000 to 15,000 nucleotides, 10,000 to 15,000 nucleotides, 1,000 to 10,000 nucleotides, 3,000 to 10,000 nucleotides, 5,000 to 10,000 nucleotides, 7,000 to 10,000 nucleotides, 1,000 to 7,000 nucleotides, 3,000 to 7,000 nucleotides, 5,000 to 7,000 nucleotides, 1,000 to 5,000 nucleotides, 3,000 to 5,000 nucleotides, or 1,000 to 3,000 nucleotides, in length. In some embodiments, the nucleic acid molecule is 6,000 to 9,000 nucleotides in length. In some embodiments, the nucleic acid molecule is 7,000 to 8,000 nucleotides in length.

In some embodiments, the LNP has a lipid (L) to nucleic acid molecule (N) mass ratio of between 10:1 and 60:1, between 20:1 and 60:1, between 30:1 and 60:1, between 40:1 and 60:1, between 50:1 and 60:1, between 10:1 and 50:1, between 20:1 and 50:1, between 30:1 and 50:1, between 40:1 and 50:1, between 10:1 and 40:1, between 20:1 and 40:1, between 30:1 and 40:1, between 10:1 and 30:1, between 20:1 and 30:1, or between 10:1 and 20:1, inclusive of all endpoints. In some embodiments, the LNP has a lipid: nucleic acid molecule mass ratio of between 30:1 and 40:1. In some embodiments, the LNP has a lipid: nucleic acid molecule mass ratio of between 30:1 and 36:1.

In some embodiments, the LNP comprises a recombinant nucleic acid molecule described herein and has a mass ratio of lipid (L) to nucleic acid (N) of about 10:1 to about 60:1. In some embodiments, the LNP comprises a recombinant nucleic acid molecule described herein and has a mass ratio of lipid (L) to nucleic acid (N) of about 20:1. In some embodiments, the LNP comprises a recombinant nucleic acid molecule described herein and has a mass ratio of lipid (L) to nucleic acid (N) of about 30:1. In some embodiments, the LNP comprises a recombinant nucleic acid molecule described herein and has a mass ratio of lipid (L) to nucleic acid (N) of about 40:1. In some embodiments, the LNP comprises a recombinant nucleic acid molecule described herein and has an L:N mass ratio of about 15:1, about 16:1, about 17:1, about 18:1, about 19:1, about 20:1, about 21:1, about 22:1, about 23:1, about 24:1, about 25:1, about 26:1, about 27:1, about 28:1, about 29:1, about 30:1, about 31:1, about 32:1, about 33:1, about 34:1, about 35:1, about 36:1, about 237:1, about 28:1, about 39:1, about 40:1, about 41:1, about 42:1, about 43:1, about 44:1, or about 45:1.

In some embodiments, the LNP comprises a nucleic acid molecule and has a lipid-nitrogen-to-phosphate ratio (N:P) of between 1 to 25. In some embodiments, the N:P is between 1 to 25, between 1 to 20, between 1 to 15, between 1 to 10, between 1 to 5, between 5 to 25, between 5 to 20, between 5 to 15, between 5 to 10, between 10 to 25, between 10 to 20, between 10 to 15, between 15 to 25, between 15 to 20, or between 20 to 25. In some embodiments, the N:P is about 1, about 2, about 3, about 4, about 5, about 6, about 7, about 8, about 9, about 10, about 11, about 12, about 13, about 14, about 15, about 16, about 17, about 18, about 19, 20, about 21, about 22, about 23, about 24, or about 25. In some embodiments, the N:P is about 8.5. In some embodiments, the N:P is about 9.

### Synthetic RNA Viral Genomes

In some embodiments, the nucleic acid payload molecule is a polynucleotide encoding for a virus. In some embodiments, the polynucleotide comprises a partial genome of a virus. In some embodiments, a replication-competent viral genome is a genome of a DNA virus or a genome of an RNA virus. In some embodiments, a replication-competent viral genome is a genome of adenovirus. In some embodiments, a DNA genome or RNA genome is a double-stranded or a single-stranded virus. In some embodiments, a replication-competent virus is selected from the group consisting of an adenovirus, a coxsackievirus, an equine herpes virus, a herpes simplex virus, an influenza virus, a lassa virus, a maraba virus, a measles virus, a murine leukemia virus, a myxoma virus, a newcastle disease virus, a orthomyxovirus, a parvovirus, a polio virus (including a chimeric polio virus such as PVS-RIPO), a reovirus, a seneca valley virus (e.g., Senecavirus A), an alphavirus, including a sindbis virus, a chikungunya virus, a Venezuelan Equine Encephalitis virus and a semliki forest virus, a vaccinia virus, and a vesicular stomatitis virus. In some embodiments, an encoded virus is a single-stranded RNA (ssRNA) virus. In some embodiments, an ssRNA virus is a positive sense ((+)-sense) or a negative-sense ((−)-sense) ssRNA virus. In some embodiments, an (+)-sense ssRNA virus is a Picomavirus. In some embodiments, a Picornavirus is a Seneca Valley Virus (SVV) or a Coxsackievirus. In some embodiments, an encoded virus is Coxsackievirus A21 (CVA21). In some embodiments, an encoded virus is selected from the group consisting of a hybrid virus (e.g., a pseudotyped virus), alphavirus (e.g., Sindbis virus, chikungunya virus, Venezuelan Equine Encephalitis virus and Semliki Forest virus) and replicon of picorna and alphavirus. In some embodiments, the polynucleotide is a modified virus RNA encoding for virus and/or proinflammatory molecules (e.g., cytokines, chemokines, antibodies, bispecific, viral and cancer antigen encoding nucleotides). In some embodiments, a polynucleotide further comprises a polynucleotide sequence encoding an exogenous payload protein. In some embodiments, a polynucleotide is an mRNA encoding for a viral antigen, a tumor antigen, a cytokine, an antibody or a bispecific antibody. In some embodiments, an exogenous payload protein is a fluorescent protein, an enzymatic protein, a cytokine, a chemokine, a ligand for a cell-surface receptor, or an antigen-binding molecule capable of binding to a cell surface receptor.

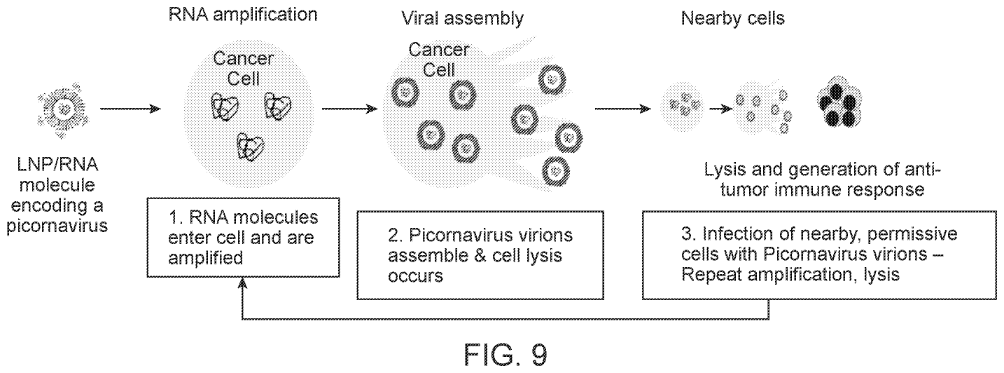

In some embodiments, the nucleic acid payload molecule is a recombinant RNA molecule encoding an oncolytic virus (e.g., an RNA genome) viral genome. Such recombinant RNA molecules are referred to herein as “synthetic viral genomes” or “synthetic RNA viral genomes”. In such embodiments, the synthetic RNA viral genome is capable of producing an infectious, lytic virus when introduced into a cell by a non-viral delivery vehicle and does not require additional exogenous genes or proteins to be present in the cell in order to replicate and produce an infectious virus. Rather, the endogenous translational mechanisms in the host cell mediate expression of the viral proteins from the synthetic RNA viral genome. The expressed viral proteins then mediate viral replication and assembly into an infectious viral particle (which may comprise a capsid protein, an envelope protein, and/or a membrane protein) comprising the RNA viral genome. As such, the RNA polynucleotides described herein (i.e., the synthetic RNA viral genomes), when introduced into a host cell, produce a virus that is capable of infecting another host cell. In some embodiments, the oncolytic virus is a picomavirus (see schematic in FIG. 9). In some embodiments, the picomavirus is a CVA21. In some embodiments, the picomavirus is an SVV.

In some embodiments, the synthetic RNA viral genome is a replicon, a RNA viral genome encoding a transgene, an mRNA molecule, or a circular RNA molecule (circRNA). In some embodiments, the synthetic RNA viral genome comprises a single stranded RNA (ssRNA) viral genome. In some embodiments, the single-stranded genome may be a positive sense or negative sense genome.

The synthetic RNA viral genomes described herein encode an oncolytic virus. Examples of oncolytic viruses are known in the art including, but not limited to a picomavirus (e.g., a coxsackievirus), a polio virus, a measles virus, a vesicular stomatitis virus, an orthomyxovirus, and a maraba virus. In some embodiments, the oncolytic virus encoded by the synthetic RNA viral genome is a virus in the family Picornaviridae family such as a coxsackievirus, a polio virus (including a chimeric polio virus such as PVS—RIPO and other chimeric Picomaviruses), or a Seneca valley virus, or any virus of chimeric origin from any multitude of picomaviruses, a virus in the Arenaviridae family such a lassa virus, a virus in the Retroviridae family such as a murine leukemia virus, a virus in the family Orthomyxoviridae such as influenza A virus, a virus in the family Paramyxoviridae such as Newcastle disease virus or measles virus, a virus in the Reoviridae family such as mammalian orthoreovirus, a virus in the Togaviridae family such as sindbis virus, or a virus in the Rhabdoviridae family such as vesicular stomatitis virus (VSV) or a maraba virus.

In some embodiments, the synthetic RNA viral genomes described herein encode a single-stranded RNA (ssRNA) viral genome. In some embodiments, the ssRNA virus is a positive-sense, ssRNA (+sense ssRNA) virus. Exemplary+sense ssRNA viruses include members of the Picornaviridae family (e.g. coxsackievirus, poliovirus, and Seneca Valley virus (SVV), including SVV-A), the Coronaviridae family (e.g., Alphacoronaviruses such as HCoV-229E and HCoV-NL63, Betacoronoaviruses such as HCoV-HKU1, HCoV-OC3, and MERS-CoV), the Retroviridae family (e.g., Murine leukemia virus), and the Togaviridae family (e.g., Sindbis virus). Additional exemplary genera and species of positive-sense, ssRNA viruses are shown below in Table 3.

TABLE 3
Positive-sense ssRNA Viruses

| Family/ | | | |
|---|---|---|---|
| Subfamily | Genus | Natural Host | Species |
| Picornaviridae | Cardiovirus | Human | |
| | Cosavirus | Human | |
| | Enterovirus | Human | Coxsackievirus |
| | | Human | Poliovirus |
| | Hepatovirus | Human | |
| | Kobuvirus | Human | |
| | Parechovirus | Human | |
| | Rosavirus | Human | |
| | Salivirus | Human | |
| | Pasivirus | Pigs | |
| | Senecavirus | Pigs | Seneca Valley |
| | | | Virus A |
| Caliciviridae | Sapovirus | Human | |
| | Norovirus | Human | |
| | Nebovirus | Bovine | |
| | Vesivirus | Felines/Swine | |
| Hepeviridae | Orthohepevirus | | |
| Astroviridae | Mamastrovirus | Human | |
| | Avastrovirus | Birds | |
| Flaviviridae | Hepacivirus | Human | |
| | Flavivirus | Arthropod | |
| | Pegivirus | | |
| | Pestivirus | Mammals | |
| Coronaviridae/ | Alphacoronavirus | | HCoV-229E |
| Coronavirinae | | | HCoV-NL63 |
| | Betacoronavirus | | HCoV-HKU1 |
| | | | HCoV-OC3 |
| | | | MERS-CoV |
| | Deltacoronavirus | | |
| | Gammacoronavirus | | |
| Coronaviridae/ | Bafinivirus | | |
| Torovirinae | Torovirus | | |
| Retroviridae | Gammaretrovirus | | Murine leukemia |
| | | | virus |
| Togaviridae | Alphavirus | | Sindbis virus |
| | | | Semliki Forest |
| | | | virus |

In some embodiments, the recombinant RNA molecules described herein encode a Picornavirus selected from a coxsackievirus, poliovirus, and Seneca Valley virus (SVV). In some embodiments, the recombinant RNA molecules described herein encode a coxsackievirus.

In some embodiments, the synthetic RNA viral genome described herein encode a Seneca Valley virus (SVV).

In some embodiments, the synthetic RNA viral genomes described herein encode a coxsackievirus. In some embodiments, the coxsackievirus is selected from CVB3, CVA21, and CVA9. The nucleic acid sequences of exemplary coxsackieviruses are provided GenBank Reference No. M33854.1 (CVB3), GenBank Reference No. KT161266.1 (CVA21), and GenBank Reference No. D00627.1 (CVA9).

In some embodiments, the payload molecule encodes an oncolytic virus. In some embodiments, the oncolytic virus is, or is derived from, Coxsackievirus, Seneca Valley virus, Togaviridae, or Alphavirus (such as Sindbis virus, Semliki Forest virus, Ross River virus, or Chikungunya virus). In some embodiments, the oncolytic virus is, or is derived from, Coxsackievirus A21 (CVA21). In some embodiments, the oncolytic virus is, or is derived from, Seneca Valley virus (SVV).

### Other Payload Molecules

The LNP of the disclosure may comprise a payload molecule selected from the group consisting of a nucleic acid, a polypeptide, a small molecule, a carbohydrate, an enzyme, a dye, a fluorochrome, and a combination thereof. In some embodiments, the LNP of the disclosure comprises a combination of payload molecules. The combination of payload molecules may be covalently linked, non-covalently associated, or have no association. Non-limiting examples of combinations of payload molecules include an antibody-drug conjugate and a Cas protein/gRNA complex.

In some embodiments, the payload molecule may be a Cas protein/gRNA complex. CRISPR (Clustered Regularly Interspaced Short Palindromic Repeats)/Cas (CRISPR Associated) nuclease system is an engineered nuclease system based on a bacterial system that can be used for mammalian genome engineering. Generally, the system comprises a Cas protein (Cas nuclease) and a guide RNA (gRNA). The gRNA is comprised of two parts; a crispr-RNA (crRNA) that is specific for a target genomic DNA sequence, and a tracr RNA (trRNA) that facilitates Cas binding. The crRNA and trRNA may be present as separate RNA oligonucleotides, or may be present in the same RNA oligonucleotide, referred to as a single guide-RNA (sgRNA). As used herein, the term “guide RNA” or “gRNA” refers to either the combination of an individual trRNA and an individual crRNA or an sgRNA. See, e.g., Jinek et al. (2012) Science 337:816-821; Cong et al. (2013) Science 339:819-823; and Ran et al. (2013) Nature Protocols 8(11):2281-2308; U.S. Patent Publication Nos. 2010-0093617, 2013-0011828, 2010-0257638, 2010-0076057, 2011-0217739, 2011-0300538, 2013-0288251, and 2012-0277120; and U.S. Pat. No. 8,546,553, each of which is incorporated herein by reference in its entirety.

In some embodiments, the payload molecule may be a base editing enzyme (e.g., cytidine deaminase or adenosine deaminase). In some embodiments, the base editing enzyme is fused to a CRISPR protein. In some embodiments, the CRISPR protein is bound to a guide RNA.

### Pharmaceutical Compositions

In some embodiments, the present disclosure includes a pharmaceutical composition comprising a compound of Formula (I) and a pharmaceutically acceptable carrier, adjuvant, or vehicle. In some embodiments, the present disclosure includes a pharmaceutical composition comprising a compound selected from Table 1 and a pharmaceutically acceptable carrier, adjuvant, or vehicle. In some embodiments, the present disclosure includes a pharmaceutical composition comprising a lipid nanoparticle (LNP) comprising a compound of Formula (I). In some embodiments, the present disclosure includes a pharmaceutical composition comprising a LNP comprising a compound of selected from Table 1. In some embodiments, the present disclosure includes a pharmaceutical composition comprising a lipid nanoparticle (LNP) comprising a compound of Formula (A), (A′), or (A″). In some embodiments, the present disclosure includes a pharmaceutical composition comprising a LNP of the present disclosure and a pharmaceutically acceptable excipient, carrier or diluent. In some embodiments, a pharmaceutical composition may comprise: (i) an LNP of the disclosure and, optionally, a payload molecule; and (ii) a pharmaceutically acceptable carrier, diluent or excipient.

A pharmaceutical composition can be formulated according to known methods to prepare pharmaceutically useful compositions, whereby the therapeutic molecule is combined in a mixture with a pharmaceutically acceptable carrier, diluent, or excipient. A carrier is said to be a “pharmaceutically acceptable carrier” if its administration can be tolerated by a recipient subject. Sterile phosphate-buffered saline is one example of a pharmaceutically acceptable carrier. Other suitable carriers, diluents, or excipients are well-known to those in the art. (See, e.g., Gennaro (ed.), Remington's Pharmaceutical Sciences (Mack Publishing Company, 19th ed. 1995).) Formulations can further include one or more excipients, preservatives, solubilizers, buffering agents, albumin to prevent protein loss on vial surfaces, etc.

A pharmaceutical composition comprising LNPs of the disclosure may be formulated in a dosage form selected from the group consisting of: an oral unit dosage form, an intravenous unit dosage form, an intranasal unit dosage form, a suppository unit dosage form, an intradermal unit dosage form, an intramuscular unit dosage form, an intraperitoneal unit dosage form, a subcutaneous unit dosage form, an epidural unit dosage form, a sublingual unit dosage form, and an intracerebral unit dosage form. The oral unit dosage form may be selected from the group consisting of: tablets, pills, pellets, capsules, powders, lozenges, granules, solutions, suspensions, emulsions, syrups, elixirs, sustained-release formulations, aerosols, and sprays.

A pharmaceutical composition may be administered to a subject in a therapeutically effective amount. In prophylactic applications, pharmaceutical compositions comprising an LNP and optionally a payload molecule of the disclosure are administered to a subject susceptible to, or otherwise at risk of, a particular disorder in an amount sufficient to eliminate or reduce the risk or delay the onset of the disorder. In therapeutic applications, compositions comprising an LNP and optionally a payload molecule of the disclosure are administered to a subject suspected of, or already suffering from such a disorder in an amount sufficient to cure, or at least partially arrest, the symptoms of the disorder and its complications. An amount adequate to accomplish this is referred to as a therapeutically effective dose or amount. In both prophylactic and therapeutic regimes, payload molecules can be administered in several dosages until a sufficient response has been achieved. Typically, the response is monitored and repeated dosages are given if the desired response starts to fade.

According to the methods of the disclosure, a composition can be administered to subjects by a variety of administration modes, including, for example, by intramuscular, subcutaneous, intravenous, intra-atrial, intra-articular, parenteral, intranasal, intrapulmonary, transdermal, intrapleural, intrathecal, intratumoral, and oral routes of administration. For prevention and treatment purposes, a composition can be administered to a subject in a single bolus delivery, via continuous delivery (e.g., continuous transdermal delivery) over an extended time period, or in a repeated administration protocol (e.g., on an hourly, daily, weekly, or monthly basis).

Administration can occur by injection, irrigation, inhalation, consumption, electro-osmosis, hemodialysis, iontophoresis, and other methods known in the art. The route of administration will vary, naturally, with the location and nature of the disease being treated, and may include, for example auricular, buccal, conjunctival, cutaneous, dental, endocervical, endosinusial, endotracheal, enteral, epidural, interstitial, intra-articular, intra-arterial, intra-abdominal, intraauricular, intrabiliary, intrabronchial, intrabursal, intracavemous, intracerebral, intracisternal, intracomeal, intracronal, intracoronary, intracranial, intradermal, intradiscal, intraductal, intraduodenal, intraduodenal, intradural, intraepicardial, intraepidermal, intraesophageal, intragastric, intragingival, intrahepatic, intraileal, intralesional, intralingual, intraluminal, intralymphatic, intramammary, intramedulleray, intrameningeal, instramuscular, intranasal, intranodal, intraocular, intraomentum, intraovarian, intraperitoneal, intrapericardial, intrapleural, intraprostatic, intrapulmonary, intraruminal, intrasinal, intraspinal, intrasynovial, intratendinous, intratesticular, intratracheal, intrathecal, intrathoracic, intratubular, intratumoral, intratympanic, intrauterine, intraperitoneal, intravascular, intraventricular, intravesical, intravestibular, intravenous, intravitreal, larangeal, nasal, nasogastric, oral, ophthalmic, oropharyngeal, parenteral, percutaneous, periarticular, peridural, perineural, periodontal, respiratory, retrotubular, rectal, spinal, subarachnoid, subconjunctival, subcutaneous, subdermal, subgingival, sublingual, submucosal, subretinal, topical, transdermal, transendocardial, transmucosal, transplacental, trantracheal, transtympanic, ureteral, urethral, and/or vaginal perfusion, lavage, direct injection, and oral administration.

In some embodiments, the pharmaceutical composition is formulated for systemic administration. In some embodiments, the systemic administration comprises intravenous administration, intra-arterial administration, intraperitoneal administration, intramuscular administration, intradermal administration, subcutaneous administration, intranasal administration, oral administration, or a combination thereof. In some embodiments, the pharmaceutical composition is formulated for intravenous administration. In some embodiments, the pharmaceutical composition is formulated for local administration. In some embodiments, the pharmaceutical composition is formulated for intratumoral administration.

Effective doses of the compositions of the disclosure vary depending upon many different factors, including means of administration, target site, physiological state of the subject, whether the subject is human or an animal, other medications administered, whether treatment is prophylactic or therapeutic, as well as the specific activity of the composition itself and its ability to elicit the desired response in the individual. In some embodiments, the subject is a human. In some embodiments, the subject can be a nonhuman mammal. Typically, dosage regimens are adjusted to provide an optimum therapeutic response, i.e., to optimize safety and efficacy.

Determination of effective dosages in this context is typically based on animal model studies followed up by human clinical trials and is guided by determining effective dosages and administration protocols that significantly reduce the occurrence or severity of the subject disorder in model subjects. Compositions of the disclosure may be suitably administered to the subject at one time or over a series of treatments and may be administered to the subject at any time from diagnosis onwards. Compositions of the disclosure may be administered as the sole treatment, as a monotherapy, or in conjunction with other drugs or therapies, as a combinatorial therapy, useful in treating the condition in question.

Dosage of the pharmaceutical composition can be varied by the attending clinician to maintain a desired concentration at a target site. Higher or lower concentrations can be selected based on the mode of delivery. Dosage should also be adjusted based on the release rate of the administered formulation.

In some embodiments, the pharmaceutical composition of the disclosure is administered to a subject for multiple times (e.g., multiple doses). In some embodiments, the pharmaceutical composition is administered two or more times, three or more times, four or more times, etc. In some embodiments, administration of the pharmaceutical composition may be repeated once, twice, 3, 4, 5, 6, 7, 8, 9, 10, or more times. The pharmaceutical composition may be administered chronically or acutely, depending on its intended purpose.

In some embodiments, the therapeutically effective amount of a composition of the disclosure is between about 1 ng/kg body weight to about 100 mg/kg body weight. In some embodiments, the range of a composition of the disclosure administered is from about 1 ng/kg body weight to about 1 μg/kg body weight, about 1 ng/kg body weight to about 100 ng/kg body weight, about 1 ng/kg body weight to about 10 ng/kg body weight, about 10 ng/kg body weight to about 1 μg/kg body weight, about 10 ng/kg body weight to about 100 ng/kg body weight, about 100 ng/kg body weight to about 1 μg/kg body weight, about 100 ng/kg body weight to about 10 μg/kg body weight, about 1 μg/kg body weight to about 10 μg/kg body weight, about 1 μg/kg body weight to about 100 μg/kg body weight, about 10 μg/kg body weight to about 100 μg/kg body weight, about 10 μg/kg body weight to about 1 mg/kg body weight, about 100 μg/kg body weight to about 10 mg/kg body weight, about 1 mg/kg body weight to about 100 mg/kg body weight, or about 10 mg/kg body weight to about 100 mg/kg body weight. Dosages within this range can be achieved by single or multiple administrations, including, e.g., multiple administrations per day or daily, weekly, bi-weekly, or monthly administrations. Compositions of the disclosure may be administered, as appropriate or indicated, as a single dose by bolus or by continuous infusion, or as multiple doses by bolus or by continuous infusion. Multiple doses may be administered, for example, multiple times per day, once daily, every 2, 3, 4, 5, 6 or 7 days, weekly, every 2, 3, 4, 5 or 6 weeks or monthly. In some embodiments, a composition of the disclosure is administered weekly. In some embodiments, a composition of the disclosure is administered biweekly. In some embodiments, a composition of the disclosure is administered every three weeks. However, other dosage regimens may be useful. The progress of this therapy is easily monitored by conventional techniques.

For administration to a human adult subject, the therapeutically effective amount may be administered in doses in the range of 0.0006 mg to 1000 mg per dose, including but not limited to 0.0006 mg per dose, 0.001 mg per dose, 0.003 mg per dose, 0.006 mg per dose, 0.01 mg per dose, 0.03 mg per dose, 0.06 mg per dose, 0.1 mg per dose, 0.3 mg per dose, 0.6 mg per dose, 1 mg per dose, 3 mg per dose, 6 mg per dose, 10 mg per dose, 30 mg per dose, 60 mg per dose, 100 mg per dose, 300 mg per dose, 600 mg per dose and 1000 mg per dose, and multiple, usually consecutive daily doses may be administered in a course of treatment. In some embodiments, a composition of the disclosure is administered at a dose level of about 0.001 mg/kg/dose to about 10 mg/kg/dose, about 0.001 mg/kg/dose to about 6 mg/kg/dose, about 0.001 mg/kg/dose to about 3 mg/kg/dose, about 0.001 mg/kg/dose to about 1 mg/kg/dose, about 0.001 mg/kg/dose to about 0.6 mg/kg/dose, about 0.001 mg/kg/dose to about 0.3 mg/kg/dose, about 0.001 mg/kg/dose to about 0.1 mg/kg/dose, about 0.001 mg/kg/dose to about 0.06 mg/kg/dose, about 0.001 mg/kg/dose to about 0.03 mg/kg/dose, about 0.001 mg/kg/dose to about 0.01 mg/kg/dose, about 0.001 mg/kg/dose to about 0.006 mg/kg/dose, about 0.001 mg/kg/dose to about 0.003 mg/kg/dose, about 0.003 mg/kg/dose to about 10 mg/kg/dose, about 0.003 mg/kg/dose to about 6 mg/kg/dose, about 0.003 mg/kg/dose to about 3 mg/kg/dose, about 0.003 mg/kg/dose to about 1 mg/kg/dose, about 0.003 mg/kg/dose to about 0.6 mg/kg/dose, about 0.003 mg/kg/dose to about 0.3 mg/kg/dose, about 0.003 mg/kg/dose to about 0.1 mg/kg/dose, about 0.003 mg/kg/dose to about 0.06 mg/kg/dose, about 0.003 mg/kg/dose to about 0.03 mg/kg/dose, about 0.003 mg/kg/dose to about 0.01 mg/kg/dose, about 0.003 mg/kg/dose to about 0.006 mg/kg/dose, about 0.006 mg/kg/dose to about 10 mg/kg/dose, about 0.006 mg/kg/dose to about 6 mg/kg/dose, about 0.006 mg/kg/dose to about 3 mg/kg/dose, about 0.006 mg/kg/dose to about 1 mg/kg/dose, about 0.006 mg/kg/dose to about 0.6 mg/kg/dose, about 0.006 mg/kg/dose to about 0.3 mg/kg/dose, about 0.006 mg/kg/dose to about 0.1 mg/kg/dose, about 0.006 mg/kg/dose to about 0.06 mg/kg/dose, about 0.006 mg/kg/dose to about 0.03 mg/kg/dose, about 0.006 mg/kg/dose to about 0.01 mg/kg/dose, about 0.01 mg/kg/dose to about 10 mg/kg/dose, about 0.01 mg/kg/dose to about 6 mg/kg/dose, about 0.01 mg/kg/dose to about 3 mg/kg/dose, about 0.01 mg/kg/dose to about 1 mg/kg/dose, about 0.01 mg/kg/dose to about 0.6 mg/kg/dose, about 0.01 mg/kg/dose to about 0.3 mg/kg/dose, about 0.01 mg/kg/dose to about 0.1 mg/kg/dose, about 0.01 mg/kg/dose to about 0.06 mg/kg/dose, about 0.01 mg/kg/dose to about 0.03 mg/kg/dose, about 0.03 mg/kg/dose to about 10 mg/kg/dose, about 0.03 mg/kg/dose to about 6 mg/kg/dose, about 0.03 mg/kg/dose to about 3 mg/kg/dose, about 0.03 mg/kg/dose to about 1 mg/kg/dose, about 0.03 mg/kg/dose to about 0.6 mg/kg/dose, about 0.03 mg/kg/dose to about 0.3 mg/kg/dose, about 0.03 mg/kg/dose to about 0.1 mg/kg/dose, about 0.03 mg/kg/dose to about 0.06 mg/kg/dose, about 0.06 mg/kg/dose to about 10 mg/kg/dose, about 0.06 mg/kg/dose to about 6 mg/kg/dose, about 0.06 mg/kg/dose to about 3 mg/kg/dose, about 0.06 mg/kg/dose to about 1 mg/kg/dose, about 0.06 mg/kg/dose to about 0.6 mg/kg/dose, about 0.06 mg/kg/dose to about 0.3 mg/kg/dose, about 0.06 mg/kg/dose to about 0.1 mg/kg/dose, about 0.1 mg/kg/dose to about 10 mg/kg/dose, about 0.1 mg/kg/dose to about 6 mg/kg/dose, about 0.1 mg/kg/dose to about 3 mg/kg/dose, about 0.1 mg/kg/dose to about 1 mg/kg/dose, about 0.1 mg/kg/dose to about 0.6 mg/kg/dose, about 0.1 mg/kg/dose to about 0.3 mg/kg/dose, about 0.3 mg/kg/dose to about 10 mg/kg/dose, about 0.3 mg/kg/dose to about 6 mg/kg/dose, about 0.3 mg/kg/dose to about 3 mg/kg/dose, about 0.3 mg/kg/dose to about 1 mg/kg/dose, about 0.3 mg/kg/dose to about 0.6 mg/kg/dose, about 0.6 mg/kg/dose to about 10 mg/kg/dose, about 0.6 mg/kg/dose to about 6 mg/kg/dose, about 0.6 mg/kg/dose to about 3 mg/kg/dose, about 0.6 mg/kg/dose to about 1 mg/kg/dose, about 1 mg/kg/dose to about 10 mg/kg/dose, about 1 mg/kg/dose to about 6 mg/kg/dose, about 1 mg/kg/dose to about 3 mg/kg/dose, about 3 mg/kg/dose to about 10 mg/kg/dose, about 3 mg/kg/dose to about 6 mg/kg/dose, or about 6 mg/kg/dose to about 10 mg/kg/dose. In some embodiments, a composition of the disclosure is administered at a dose level of about 0.001 mg/kg/dose, about 0.003 mg/kg/dose, about 0.006 mg/kg/dose, about 0.01 mg/kg/dose, about 0.03 mg/kg/dose, about 0.06 mg/kg/dose, about 0.1 mg/kg/dose, about 0.3 mg/kg/dose, about 0.6 mg/kg/dose, about 1 mg/kg/dose, about 3 mg/kg/dose, about 6 mg/kg/dose, or about 10 mg/kg/dose. Compositions of the disclosure can be administered at different times of the day. In one embodiment the optimal therapeutic dose can be administered in the evening. In another embodiment the optimal therapeutic dose can be administered in the morning. As expected, the dosage will be dependent on the condition, size, age, and condition of the subject.

Dosage of the pharmaceutical composition can be varied by the attending clinician to maintain a desired concentration at a target site. Higher or lower concentrations can be selected based on the mode of delivery. Dosage should also be adjusted based on the release rate of the administered formulation.

In some embodiments, the pharmaceutical composition of the disclosure is administered to a subject for multiple times (e.g., multiple doses). In some embodiments, the pharmaceutical composition is administered two or more times, three or more times, four or more times, etc. In some embodiments, administration of the pharmaceutical composition may be repeated once, twice, 3, 4, 5, 6, 7, 8, 9, 10, or more times. The pharmaceutical composition may be administered chronically or acutely, depending on its intended purpose.

In some embodiments, the interval between two consecutive doses of the pharmaceutical composition is less than 4, less than 3, less than 2, or less than 1 weeks. In some embodiments, the interval between two consecutive doses is less than 3 weeks. In some embodiments, the interval between two consecutive doses is less than 2 weeks. In some embodiments, the interval between two consecutive doses is less than 1 week. In some embodiments, the interval between two consecutive doses is less than 28, 27, 26, 25, 24, 23, 22, 21, 20, 19, 18, 17, 16, 15, 14, 13, 12, 11, 10, 9, 8, 7, 6, 5, 4, 3, 2 or 1 days. In some embodiments, the interval between two consecutive doses of the pharmaceutical composition is at least 4, at least 3, at least 2, or at least 1 weeks. In some embodiments, the interval between two consecutive doses of the pharmaceutical composition of the disclosure is at least 3 weeks. In some embodiments, the interval between two consecutive doses of the pharmaceutical composition of the disclosure is at least 2 weeks. In some embodiments, the interval between two consecutive doses of the pharmaceutical composition of the disclosure is at least 1 week. In some embodiments, the interval between two consecutive doses of the pharmaceutical composition of the disclosure is at least 28, 27, 26, 25, 24, 23, 22, 21, 20, 19, 18, 17, 16, 15, 14, 13, 12, 11, 10, 9, 8, 7, 6, 5, 4, 3, 2 or 1 days. In some embodiments, the subject is administered a dose of the pharmaceutical composition of the disclosure once daily, every 2, 3, 4, 5, 6, 7, 8, 9, 10, 11, 12, 13, 14, 15, 16, 17, 18, 19, 20, 21, 22, 23, 24, 25, 26, 27, or 28 days. In some embodiments, the subject is administered a dose of the pharmaceutical composition of the disclosure once every 4, 5, 6, 7, 8, 9, 10, 11, or 12 weeks. In some embodiments, the subject is administered a dose of the pharmaceutical composition of the disclosure once every 1, 2, 3, 4, 5, 6, 7, 8, 9, 10, 11, or 12 months.

In some embodiments, the pharmaceutical composition of the disclosure is administered multiple times, wherein the serum half-life of the LNP in the subject following the second and/or subsequent administration is at least 40%, 50%, 60%, 70%, 80%, 85%, 90%, or 95% of the serum half-life of the LNP following the first administration.

In some embodiments, the second and subsequent doses of the pharmaceutical composition comprising an payload molecule may maintain an activity of the payload molecule of at least 50% of the activity of the first dose, or at least 60% of the first dose, or at least 70% of the first dose, or at least 75% of the first dose, or at least 80% of the first dose, or at least 85% of the first dose, or at least 90% of the first dose, or at least 95% of the first dose, or more, for at least 1 day, 2 days, 3 days, 4 days, 5 days, 6 days, or 7 days after second administration or subsequent administration.

In some embodiments, the pharmaceutical composition of the disclosure has an duration of therapeutic effect in vivo of about 1 hour or longer, about 2 hours or longer, about 3 hours or longer, about 4 hours or longer, about 5 hours or longer, about 6 hours or longer, about 7 hours or longer, about 8 hours or longer, about 9 hours or longer, about 10 hours or longer, about 12 hours or longer, about 14 hours or longer, about 16 hours or longer, about 18 hours or longer, about 20 hours or longer, about 25 hours or longer, about 30 hours or longer, about 35 hours or longer, about 40 hours or longer, about 45 hours or longer, or about 50 hours or longer. In some embodiments, the pharmaceutical composition of the disclosure has an duration of therapeutic effect in vivo of at least 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 1.5 days, 2 days, 3 days, 4 days, 5 days, 6 days, 7 days, 8 days, 9 days, or 10 days.

In some embodiments, the pharmaceutical composition of the disclosure has a half-life in vivo comparable to that of a pre-determined threshold value. In some embodiments, the pharmaceutical composition of the disclosure has a half-life in vivo greater than that of a pre-determined threshold value. In some embodiments, the pharmaceutical composition of the disclosure has a half-life in vivo shorter than that of a pre-determined threshold value. In some embodiments, the pre-determined threshold value is the half-life of a control composition comprising the same payload molecule and LNP except that the LNP comprises (i) a PEG-lipid that is not of Formula (A), (A′), or (A″) (for example, the PEG-lipid of the LNP in the control composition may be PEG2k-DPG); or (ii) a cationic lipid that is not of Formula (I).

In some embodiments, the pharmaceutical composition of the disclosure has an AUC (area under the blood concentration-time curve) following a repeat dose that is at least 30%, at least 40%, at least 50%, at least 60%, at least 70%, at least 80%, at least 90%, or at least 100% of the AUC following the previous dose. In some embodiments, the pharmaceutical composition has an AUC that is at least 60% of the AUC following the previous dose. In some embodiments, following a repeat dose, AUC of the pharmaceutical composition decreases less than 70%, less than 60%, less than 60%, less than 40%, less than 30%, less than 20%, less than 10%, or less than 5% compared to the AUC following the previous dose. In some embodiments, following a repeat dose, AUC of the pharmaceutical composition decreases less than 40% compared to the AUC following the previous dose.

In some embodiments, the pharmaceutical composition of the disclosure comprises a nucleic acid molecule encoding viral genome of an oncolytic virus, and wherein administration of the pharmaceutical composition to a subject bearing a tumor delivers the nucleic acid molecule into tumor cells. In some embodiments, the nucleic acid molecule is a RNA molecule. In some embodiments, administration of the pharmaceutical composition results in replication of the oncolytic virus in tumor cells. In some embodiments, administration of the pharmaceutical composition to a subject bearing a tumor results in selective replication of the oncolytic virus in tumor cells as compared to normal cells.

In some embodiments, administration of the pharmaceutical composition of the disclosure to a subject bearing a tumor inhibits growth of the tumor. In some embodiments, administration of the pharmaceutical composition inhibits growth of the tumor for at least 1 week, at least 1 month, at least 2 months, at least 3 months, at least 4 months, at least 6 months, at least 9 months, at least 12 months, at least 2 years, or longer. In some embodiments, inhibiting growth of the tumor means controlling the size of the tumor within 100% of the size of the tumor just before administration of the pharmaceutical composition for a specified time period. In some embodiments, inhibiting growth of the tumor means controlling the size of the tumor within 110%, within 120%, within 130%, within 140%, or within 150%, of the size of the tumor just before administration of the pharmaceutical composition.

In some embodiments, administration of the pharmaceutical composition to a subject bearing a tumor leads to tumor shrinkage or elimination. In some embodiments, administration of the pharmaceutical composition leads to tumor shrinkage or elimination for at least 1 week, at least 1 month, at least 2 months, at least 3 months, at least 4 months, at least 6 months, at least 9 months, at least 12 months, at least 2 years, or longer. In some embodiments, administration of the pharmaceutical composition leads to tumor shrinkage or elimination within 1 week, within 2 weeks, within 3 weeks, within 4 weeks, within 1 month, within 2 months, within 3 months, within 4 months, within 6 months, within 9 months, within 12 months, or within 2 years. In some embodiments, tumor shrinkage means reducing the size of the tumor by at least 10%, at least 20%, at least 30%, at least 40%, at least 50%, at least 60%, at least 70%, at least 80%, at least 90%, or at least 95%, compared to the size of the tumor just before administration of the pharmaceutical composition. In some embodiments, tumor shrinkage means reducing the size of the tumor at least 30% compared to the size of the tumor just before administration of the pharmaceutical composition.

Pharmaceutical compositions can be supplied as a kit comprising a container that comprises the pharmaceutical composition as described herein. A pharmaceutical composition can be provided, for example, in the form of an injectable solution for single or multiple doses, or as a sterile powder that will be reconstituted before injection. Alternatively, such a kit can include a dry-powder disperser, liquid aerosol generator, or nebulizer for administration of a pharmaceutical composition. Such a kit can further comprise written information on indications and usage of the pharmaceutical composition

### Methods of Use

In some embodiments, the disclosure provides methods of treating a disease or disorder in a subject in need thereof, comprising administering to the subject a therapeutically effective amount of a composition (e.g., pharmaceutical composition) of the disclosure. In some embodiments, the present disclosure includes a method of treating a disease or disorder comprising administering to a patient in need thereof the lipid nanoparticle described herein. In some embodiments, the disease or disorder comprises a cancer.

The method may be a method of treating a subject having or at risk of having a condition that benefits from the payload molecule, particularly if the payload molecule is a therapeutic agent. Alternatively, the method may be a method of diagnosing a subject, in which case the payload molecule may be is a diagnostic agent.

In some embodiments, the instant disclosure includes a method of delivering a payload to a cell, comprising administering to a subject in need thereof a lipid particle or pharmaceutical composition described herein. In some embodiments, the instant disclosure includes a method a delivering a polynucleotide to a cell, comprising administering to a subject in need thereof a lipid particle or a pharmaceutical composition comprising (i) a compound of Formula (I); (ii) a compound selected from Table 1, or (iii) a compound of Formula (A), (A′), or (A″). In some embodiments, a polynucleotide encodes a polypeptide or a functional variant or fragment thereof, such that expression of the polypeptide or the functional variant or fragment thereof is increased. In another embodiment, a polynucleotide encodes an immunotherapeutic or a functional variant or fragment thereof. In some embodiments, a polynucleotide that encodes an immunotherapeutic or a functional variant or fragment thereof. In some embodiments, the present disclosure includes a polynucleotide that comprises a viral genome or a functional variant or fragment thereof. In some embodiments, a polynucleotide encodes an antigen, a protein, a CAS9 protein, or a base editing enzyme or a fusion protein thereof (e.g., a base editing enzyme fused to a CRISPR protein bound to a guide RNA). In some embodiments, the polynucleotide comprise a siRNA, saRNA, miRNA, or guide RNA.

In yet a further related embodiment, the present disclosure includes a method of treating a disease or disorder characterized by overexpression of a polypeptide in a subject, comprising providing to the subject a lipid particle or pharmaceutical composition of the present disclosure, wherein the therapeutic agent is polynucleotide.

In another related embodiment, the present disclosure includes a method of treating a disease or disorder characterized by under expression of a polypeptide in a subject.

In some embodiments, a disease or disorder is cancer. In some embodiments, cancer selected from the group consisting of lung cancer, breast cancer, ovarian cancer, cervical cancer, prostate cancer, testicular cancer, colorectal cancer, colon cancer, pancreatic cancer, liver cancer, gastric cancer, head and neck cancer, thyroid cancer, malignant glioma, glioblastoma, melanoma, Merkel cell carcinoma, B-cell lymphoma, multiple myeloma, leukemia, renal cell carcinoma, and neuroblastoma. In some embodiments, cancer is lung cancer. In some embodiments, lung cancer is small cell lung cancer or non-small cell lung cancer. In some embodiments, cancer is liver cancer. In some embodiments, liver cancer is hepatocellular carcinoma (HCC). In some embodiments, renal cancer is renal clear cell cancer (RCC). In some embodiments, renal cell carcinoma is selected from the group consisting of clear cell renal cell carcinoma, papillary renal cell carcinoma, and chromophobe renal cell carcinoma. In some embodiments, cancer is B-cell lymphoma. In some embodiments, B-cell lymphoma is selected from the group consisting of diffuse large B-cell lymphoma, follicular lymphoma, marginal zone lymphoma, and mantle cell lymphoma. In some embodiments, cancer is leukemia. In some embodiments, leukemia is selected from the group consisting of B-cell leukemia, T-cell leukemia, acute myeloid leukemia, and chronic myeloid leukemia.

In yet another embodiment, the present disclosure includes a method of treating a subject, comprising administering the pharmaceutical composition comprising polynucleotide encoding a viral, bacterial or fungal protein to the subject in an amount sufficient to cause production of antibody in serum of the subject. In some embodiments, amount of a composition administered is sufficient to produce circulating antibodies; or to produce viral-specific CD8+ T cells in a subject; or to produce antigen-specific antibody.

In other embodiments, administration is parenterally. In some embodiments, administration is by subcutaneous injection, intradermal injection, or intramuscular injection; or a pharmaceutical composition is administered at least twice. In another embodiment, a method further comprising a step of measuring antibody titer or CD8+ T cells.

In some embodiments, a pharmaceutical composition described herein comprises a nucleic acid that encodes an antibody. In some embodiments, the antibody is capable of binding a cell-associated or secreted protein or a fragment or variant of a human protein. In another embodiment, an antibody is capable of binding to a viral, bacterial or fungal particle. Another aspect of the description is a method of treating a subject, comprising administering the pharmaceutical composition comprising a nucleic acid encoding an antibody to a subject to the subject in an amount sufficient to cause production of the antibody in serum of the subject.

In various embodiments, the disclosure relates to a method of treating cancer in a subject in need thereof, comprising administering a therapeutically effective amount of a composition as described herein to the subject.

In some embodiments, the disclosure provides methods of delivering a payload molecule to a cell, the method comprising contacting the cell with the LNP or pharmaceutical composition thereof, wherein the LNP comprises the payload molecule. In some embodiments, the payload molecule is a nucleic acid molecule encoding a virus, and wherein contacting the cell with the LNP results in production of viral particles by the cell, and wherein the viral particles are infectious and lytic.

In some embodiments, the disclosure provides methods of delivering an LNP to a subject, comprising administering the LNP or the pharmaceutical composition thereof of the disclosure to the subject. In some embodiments, the method comprises multiple administrations. In some embodiments, the interval between two consecutive administrations of the pharmaceutical composition is less than 4, less than 3, less than 2, or less than 1 weeks. In some embodiments, the interval between two consecutive administrations is less than 2 weeks. In some embodiments, the interval between two consecutive administrations is less than 1 week. In some embodiments, the interval between two consecutive administrations is less than 28, 27, 26, 25, 24, 23, 22, 21, 20, 19, 18, 17, 16, 15, 14, 13, 12, 11, 10, 9, 8, 7, 6, 5, 4, 3, 2 or 1 days. In some embodiments, the interval between two consecutive administrations of the pharmaceutical composition is at least 4, at least 3, at least 2, or at least 1 weeks. In some embodiments, the interval between two consecutive administrations of the pharmaceutical composition of the disclosure is at least 2 weeks. In some embodiments, the interval between two consecutive administrations of the pharmaceutical composition of the disclosure is at least 1 week. In some embodiments, the interval between two consecutive administrations of the pharmaceutical composition of the disclosure is at least 28, 27, 26, 25, 24, 23, 22, 21, 20, 19, 18, 17, 16, 15, 14, 13, 12, 11, 10, 9, 8, 7, 6, 5, 4, 3, 2 or 1 days. In some embodiments, the method comprises administering to a subject the pharmaceutical composition of the disclosure every 1, 2, 3, 4, 5, 6, 7, 8, 9, 10, 11, 12, 13, 14, 15, 16, 17, 18, 19, 20, 21, 22, 23, 24, 25, 26, 27, or 28 days. In some embodiments, the method comprises administering to a subject the pharmaceutical composition of the disclosure once every 4, 5, 6, 7, 8, 9, 10, 11, or 12 weeks. In some embodiments, the method comprises administering to a subject the pharmaceutical composition of the disclosure once every 1, 2, 3, 4, 5, 6, 7, 8, 9, 10, 11, or 12 months.

In some embodiments, the disclosure provides methods of delivering an LNP to a subject, comprising administering the LNP or the pharmaceutical composition thereof of the disclosure to the subject, wherein the method comprises multiple administrations. In some embodiments, serum half-life of the LNP in the subject following the second and/or subsequent administration of the method is at least 40%, 50%, 60%, 70%, 80%, 85%, 90%, or 95% of the serum half-life of the LNP following the first administration.

In some embodiments, the LNP has an AUC following a repeat dose that is at least 30%, at least 40%, at least 50%, at least 60%, at least 70%, at least 80%, at least 90%, or at least 100% of the AUC following the previous dose. In some embodiments, the LNP has an AUC that is at least 60% of the AUC following the previous dose. In some embodiments, following a repeat dose, AUC of the LNP decreases less than 70%, less than 60%, less than 60%, less than 40%, less than 30%, less than 20%, less than 10%, or less than 5% compared to the AUC following the previous dose. In some embodiments, following a repeat dose, AUC of the LNP decreases less than 40% compared to the AUC following the previous dose.

In some embodiments, the disclosure provides methods of delivering an LNP to a subject, comprising administering the LNP or the pharmaceutical composition thereof of the disclosure to the subject, wherein the LNP comprises a nucleic acid molecule encoding a viral genome of an oncolytic virus, wherein the subject has a tumor, and wherein administration of the LNP delivers the nucleic acid molecule into tumor cells. In some embodiments, administration of the LNP results in replication of the oncolytic virus in tumor cells. In some embodiments, administration of the LNP results in selective replication of the oncolytic virus in tumor cells as compared to normal cells.

In some embodiments, the disclosure provides methods of delivering an LNP to a subject, comprising administering the LNP or the pharmaceutical composition thereof of the disclosure to the subject, wherein administration of the LNP to a subject bearing a tumor inhibits growth of the tumor. In some embodiments, the method inhibits growth of the tumor for at least 1 week, at least 1 month, at least 2 months, at least 3 months, at least 4 months, at least 6 months, at least 9 months, at least 12 months, at least 2 years, or longer. In some embodiments, inhibiting growth of the tumor means controlling the size of the tumor within 100% of the size of the tumor just before administration of the pharmaceutical composition for a specified time period. In some embodiments, inhibiting growth of the tumor means controlling the size of the tumor within 110%, within 120%, within 130%, within 140%, or within 150%, of the size of the tumor just before administration of the pharmaceutical composition.

In some embodiments, the disclosure provides methods of delivering an LNP to a subject, comprising administering the LNP or the pharmaceutical composition thereof of the disclosure to the subject, wherein administration of the LNP to a subject bearing a tumor leads to tumor shrinkage or elimination. In some embodiments, the method results in tumor shrinkage or elimination for at least 1 week, at least 1 month, at least 2 months, at least 3 months, at least 4 months, at least 6 months, at least 9 months, at least 12 months, at least 2 years, or longer. In some embodiments, the method results in tumor shrinkage or elimination within 1 week, within 2 weeks, within 3 weeks, within 4 weeks, within 1 month, within 2 months, within 3 months, within 4 months, within 6 months, within 9 months, within 12 months, or within 2 years. In some embodiments, tumor shrinkage means reducing the size of the tumor by at least 10%, at least 20%, at least 30%, at least 40%, at least 50%, at least 60%, at least 70%, at least 80%, at least 90%, or at least 95%, compared to the size of the tumor just before administration of the pharmaceutical composition. In some embodiments, tumor shrinkage means reducing the size of the tumor at least 30% compared to the size of the tumor just before administration of the pharmaceutical composition.

In some embodiments, the disclosure provides methods of delivering an LNP to a subject, comprising administering the LNP or the pharmaceutical composition thereof of the disclosure to the subject, wherein administration of the LNP to a subject bearing a tumor inhibits the metastasis of the cancer.

In some embodiments, the subject is a mammal. In some embodiments, the subject is a human. In some embodiments, the subject has a cancer, and wherein the method inhibits or slows the growth and/or metastasis of the cancer.

In some embodiments, the disclosure provides methods of delivering an LNP to a subject, comprising systemically administering the LNP or pharmaceutical composition thereof. In some embodiments, the administration is intravenous, intra-arterial, intraperitoneal, intramuscular, intradermal, subcutaneous, intranasal, oral, or a combination thereof.

In some embodiments, the disclosure provides methods of delivering an LNP to a subject, comprising locally administering the LNP or pharmaceutical composition thereof. In some embodiments, the administration is intratumoral.

In some embodiments, the cancer is a lung cancer, a liver cancer, a prostate cancer, a bladder cancer, a pancreatic cancer, a gastric cancer, a breast cancer, a neuroblastoma, a rhabdomyosarcoma, a medullablastoma, or a melanoma. In some embodiments, the cancer is a neuroendocrine cancer.

Examples of cancer include but are not limited to carcinoma, lymphoma, blastoma, sarcoma (including liposarcoma, osteogenic sarcoma, angiosarcoma, endotheliosarcoma, leiomyosarcoma, chordoma, lymphangiosarcoma, lymphangioendotheliosarcoma, rhabdomyosarcoma, fibrosarcoma, myxosarcoma, chondrosarcoma), neuroendocrine tumors, mesothelioma, synovioma, schwannoma, meningioma, adenocarcinoma, melanoma, and leukemia or lymphoid malignancies. More particular examples of such cancers include squamous cell cancer (e.g., epithelial squamous cell cancer), lung cancer including small-cell lung cancer, non-small cell lung cancer, adenocarcinoma of the lung and squamous carcinoma of the lung, small cell lung carcinoma, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulvar cancer, thyroid cancer, hepatic carcinoma, anal carcinoma, penile carcinoma, testicular cancer, esophageal cancer, tumors of the biliary tract, Ewing's tumor, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, testicular tumor, lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, leukemia, lymphoma, multiple myeloma, Waldenstrom's macroglobulinemia, myelodysplastic disease, heavy chain disease, neuroendocrine tumors, Schwannoma, and other carcinomas, as well as head and neck cancer. In some embodiments, the cancer is selected from small cell lung cancer (SCLC), small cell bladder cancer, large cell neuroendocrine carcinoma (LCNEC), castration-resistant small cell neuroendocrine prostate cancer (CRPC-NE), carcinoid (e.g., pulmonary carcinoid), and glioblastoma multiforme-IDH mutant (GBM-IDH mutant).

### Method of LNP Preparation

In some embodiments, the disclosure provides methods for preparing a composition of lipid nanoparticles (LNPs) containing a nucleic acid molecule, comprising the steps of
(a) diluting the nucleic acid molecule to a desired concentration in an aqueous solution;
(b) mixing organic lipid phase comprising all lipid components of the LNPs with the aqueous phase containing the nucleic acid molecule using microfluidic flow to form the LNPs;
(c) dialyzing the LNPs against a buffer to remove the organic solvent;
(d) concentrating the LNPs to a target volume; and
(e) optionally, filtered through a sterile filter.

In some embodiments, the organic lipid phase and the aqueous phase are mixed at a ratio of between 1:1 (v:v) and 1:10 (v:v). In some embodiments, the organic lipid phase and the aqueous phase are mixed at a ratio of 1:1 (v:v), 1:2 (v:v), 1:3 (v:v), 1:4 (v:v), 1:5 (v:v), 1:6 (v:v), 1:7 (v:v), 1:8 (v:v), 1:9 (v:v), or 1:10 (v:v). In some embodiments, the organic lipid phase and the aqueous phase are mixed at a ratio of between 1:1 (v:v) and 1:3 (v:v), between 1:2 (v:v) and 1:4 (v:v), between 1:3 (v:v) and 1:5 (v:v), between 1:4 (v:v) and 1:6 (v:v), between 1:5 (v:v) and 1:7 (v:v), between 1:6 (v:v) and 1:8 (v:v), between 1:7 (v:v) and 1:9 (v:v), or between 1:8 (v:v) and 1:10 (v:v). In some embodiments, the organic lipid phase and the aqueous phase are mixed at a ratio of between 1:3 (v:v) and 1:5 (v:v). In some embodiments, the organic lipid phase and the aqueous phase are mixed at a ratio of 1:3 (v:v). In some embodiments, the organic lipid phase and the aqueous phase are mixed at a ratio of 1:5 (v:v).

In some embodiments, the total flow rate of the microfluidic flow is 5-20 m/min. In some embodiments, the total flow rate of the microfluidic flow is 5, 6, 7, 8, 9, 10, 11, 12, 13, 14, 15, 16, 17, 18, 19, or 20 mL/min. In some embodiments, the total flow rate of the microfluidic flow is 9-20 mL/min. In some embodiments, the total flow rate of the microfluidic flow is 11-13 mL/min.

In some embodiments, the solvent in the organic lipid phase in step (b) is ethanol. In some embodiments, heat is applied to the organic lipid phase in step (b). In some embodiments, about 40, 45, 50, 55, 60, 65, 70, 75, or 80° C. is applied to the organic lipid phase in step (b). In some embodiments, 60° C. heat is applied to the organic lipid phase in step (b). In some embodiments, no heat is applied to the organic lipid phase in step (b).

In some embodiments, the aqueous solution in step (a) has a pH of between 1 and 7. In some embodiments, the aqueous solution in step (a) has a pH of between 1 and 3, between 2 and 4, between 3 and 5, between 4 and 6, or between 5 and 7. In some embodiments, the aqueous solution in step (a) has a pH of 1, 1.5, 2, 2.5, 3, 3.5, 4, 4.5, 5, 5.5, 6, 6.5, or 7. In some embodiments, the aqueous solution in step (a) has a pH of 3. In some embodiments, the aqueous solution in step (a) has a pH of 5.

In some embodiments, the total lipid concentration is between 5 mM and 80 mM. In some embodiments, the total lipid concentration is about 5, 10, 15, 20, 25, 30, 35, 40, 45, 50, 55, 60, 65, 70, 75, or 80 mM. In some embodiments, the total lipid concentration is about 20 mM. In some embodiments, the total lipid concentration is about 40 mM.

In some embodiments, the LNP generated by the method has a lipid-nitrogen-to-phosphate ratio (N:P) of between 1 to 25. In some embodiments, the N:P is 1, 2, 3, 4, 5, 6, 7, 8, 9, 10, 11, 12, 13, 14, 15, 16, 17, 18, 19, 20, 21, 22, 23, 24, or 25. In some embodiments, the N:P is between 1 to 25, between 1 to 20, between 1 to 15, between 1 to 10, between 1 to 5, between 5 to 25, between 5 to 20, between 5 to 15, between 5 to 10, between 10 to 25, between 10 to 20, between 10 to 15, between 15 to 25, between 15 to 20, or between 20 to 25. In some embodiments, the LNP comprises a nucleic acid molecule and has a lipid-nitrogen-to-phosphate ratio (N:P) of 14.

In some embodiments, the buffer in step (c) has a neutral pH (e.g., 1×PBS, pH 7.2). In some embodiments, step (d) uses centrifugal filtration for concentrating.

In some embodiments, the encapsulation efficiency of the method of the disclosure is at least 70%, at least 75%, at least 75%, at least 80%, at least 90%, at least 95%, at least 97%, at least 98%, or at least 99%. In some embodiments, the encapsulation efficiency of the method of the disclosure is at least 90%. In some embodiments, the encapsulation efficiency of the method of the disclosure is at least 95%. In some embodiments, the encapsulation efficiency is determined by RiboGreen.

In some embodiments, the LNPs produced by the method of the disclosure have an average size (i.e., average outer diameter) of about 50 nm to about 500 nm. In some embodiments, the LNPs have an average size of about 50 nm to about 200 nm, about 100 nm to about 200 nm, about 150 nm to about 200 nm, about 50 nm to about 100 nm, about 50 nm to about 150 nm, about 100 nm to about 150 nm, about 200 nm to about 250 nm, about 250 nm to about 300 nm, about 300 nm to about 400 nm, about 150 nm to about 500 nm, about 200 nm to about 500 nm, about 300 nm to about 500 nm, about 350 nm to about 500 nm, about 400 nm to about 500 nm, about 425 nm to about 500 nm, about 450 nm to about 500 nm, or about 475 nm to about 500 nm. In some embodiments, the plurality of LNPs have an average size of about 50 nm, 60 nm, 70 nm, 80 nm, 90 nm, 100 nm, 110 nm, about 120, or about 125 nm. In some embodiments, the plurality of LNPs have an average size of about 100 nm. In some embodiments, the plurality of LNPs have an average size of 50 nm to 150 nm. In some embodiments, the plurality of LNPs have an average size (average outer diameter) of 50 nm to 150 nm, 50 nm to 125 nm, 50 nm to 100 nm, 50 nm to 75 nm, 75 nm to 150 nm, 75 nm to 125 nm, 75 nm to 100 nm, 100 nm to 150 nm, 100 nm to 125 nm, or 125 nm to 150 nm. In some embodiments, the plurality of LNPs have an average size of 70 nm to 90 nm, 80 nm to 100 nm, 90 nm to 110 nm, 100 nm to 120 nm, 110 nm to 130 nm, 120 nm to 140 nm, or 130 nm to 150 nm. In some embodiments, the plurality of LNPs have an average size of 90 nm to 110 nm.

In some embodiments, the polydispersity index of the plurality of LNPs is between 0.01 and 0.3. In some embodiments, the polydispersity index of the plurality of LNPs is between 0.1 and 0.15. In some embodiments, the polydispersity index of the plurality of LNPs is about 0.01, about 0.02, about 0.03, about 0.04, about 0.05, about 0.06, about 0.07, about 0.08, about 0.09, about 0.10, about 0.11, about 0.12, about 0.13, about 0.14, about 0.15, about 016, about 0.17, about 0.18, about 0.19, about 0.20, about 0.21, about 0.22, about 0.23, about 0.24, about 0.25, about 0.26, about 0.27, about 0.28, about 0.29, or about 0.30. In some embodiments, the polydispersity index of the plurality of LNPs is about 0.10, about 0.11, about 0.12, about 0.13, about 0.14, or about 0.15. In some embodiments, the average diameter and/or the polydispersity is determined via dynamic light scattering.

### Exemplification

### Abbreviations:

Bn: benzyl
DCM: dichloromethane
DMAP: 4-Dimethylaminopyridine
EtOAc: ethyl acetate
EDCI 1-ethyl-3-(3-dimethylaminopropyl)carbodiimide
HPLC: high performance liquid chromatography
LCMS: liquid chromatography-mass spectrometry
Ns: nosylate
TBAI: tetrabutylammonium iodide
TEA: triethylamine (NEt₃)
THF: tetrahydrofuran
TFA: trifluoroacetic acid
Ts: tosyl
   Pharmacokinetic parameters
AUC (area under the curve): the integral of the concentration-time curve
Cₘₐₓ: the peak plasma concentration of a drug after administration
Co: amount of drug in a given volume of plasma
CL (clearance): the volume of plasma cleared of the drug per unit time
t_{1/2} (elimination half-life): the time required for the concentration of the drug to reach half of its original value
tₘₐₓ: time to reach Cₘₐₓ
Vₛₛ (steady state volume of distribution): the apparent volume in which a drug is distributed at steady state

### Example 1: Synthesis of Ionizable Lipids

### Synthesis of Intermediate A: Route 1

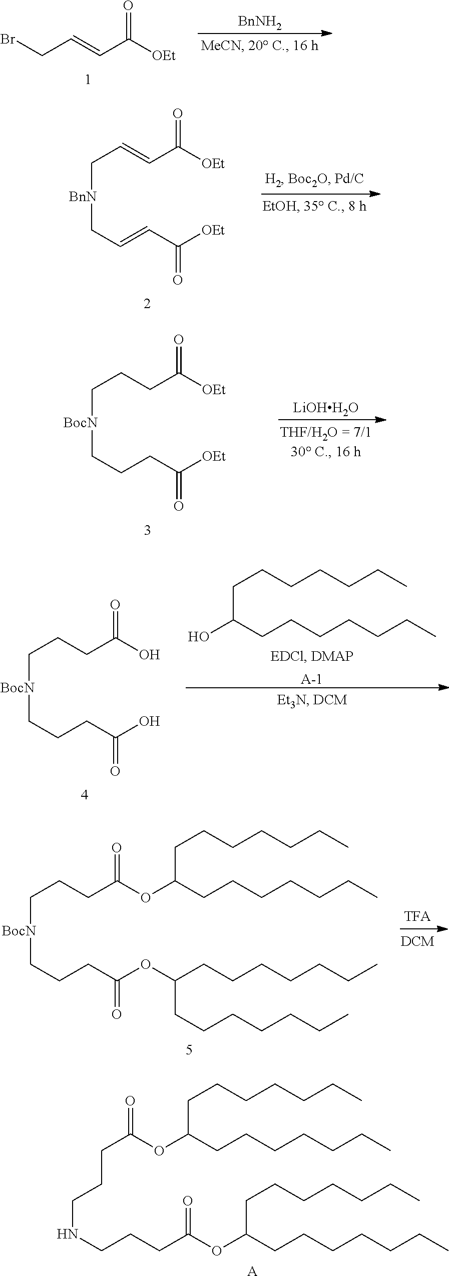

### Step 1: (2E, 2′E)-diethyl 4,4′-(benzylazanediyl)bis(but-2-enoate) (2)

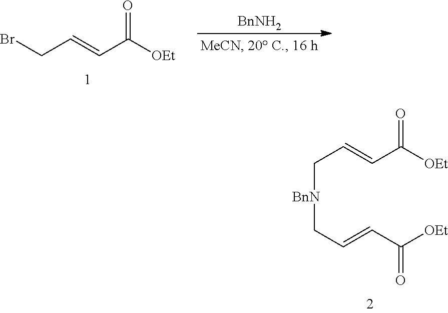 [SMILES: CCOC(=O)/C=C/CBr ; "BnNH2" ; CCOC(=O)/C=C/CN(C/C=C/C(=O)OCC)Cc1ccccc1]

To a solution of phenylmethanamine (6.94 g, 64.75 mmol, 0.5 eq) in MeCN (300 mL) were added K₂CO₃ (19.69 g, 142.46 mmol, 1.1 eq) and ethyl (E)-4-bromobut-2-enoate (25 g, 129.51 mmol, 1 eq). The mixture was stirred at 20° C. for 16 hr. The reaction mixture was filtered and the filter cake was washed with EtOAc (20 mL*2). The filtrate was concentrated in vacuum to give a residue. The residue was purified by flash silica gel chromatography (120 g SepaFlash® Silica Flash Column, EtOAc/Petroleum Ether (PE): 0˜10%) to give compound 2 (20.3 g, 56.17 mmol, 43.4% yield) as a yellow oil.

¹H NMR (400 MHz, CDCl₃) δ=7.39-7.31 (m, 4H), 7.30-7.23 (m, 1H), 6.99-6.93 (m, 2H), 6.07-6.03 (m, 2H), 4.22 (q, J=7.2, 4H), 3.63 (s, 2H), 3.24-3.23 (m, 4H), 1.32 (t, J=7.2, 6H).

### Step 2: diethyl 4,4′-((tert-butoxycarbonyl)azanediyl)dibutanoate (3)

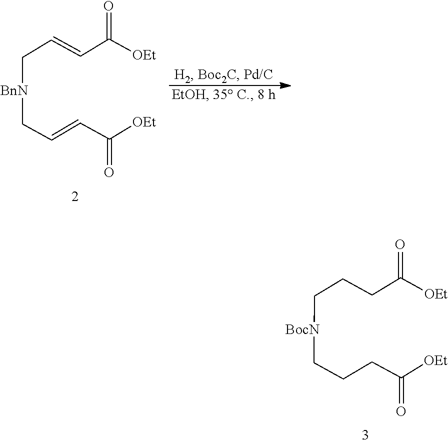 [SMILES: CCOC(=O)/C=C/CN(C/C=C/C(=O)OCC)Cc1ccccc1 ; CCOC(=O)CCCN(CCCC(=O)OCC)C(=O)OC(C)(C)C]

To a solution of ethyl (E)-4-[benzyl-[(E)-4-ethoxy-4-oxo-but-2-enyl]amino]but-2-enoate (20 g, 60.35 mmol, 1 eq) in EtOH (400 mL) was added (Boc)₂O (19.76 g, 90.52 mmol, 20.80 mL, 1.5 eq) and Pd/C (3 g, 60.35 mmol, 10% purity) under N₂. The suspension was degassed under vacuum and purged with H₂ several times. The mixture was stirred under H₂ (50 psi) at 35° C. for 8 hours. The reaction mixture was filtered and the filter cake was washed with ethanol (80 mL*2). The filtrate was concentrated in vacuum to give residue. The residue was purified by flash silica gel chromatography (120 g SepaFlash® Silica Flash Column, EtOAc/Petroleum ether (PE): 0˜15%) to give compound 3 (13.2 g, 38.21 mmol, 63.3% yield) as a yellow oil.

¹H NMR (400 MHz, CDCl₃) δ=4.17-4.12 (m, 4H), 3.25-3.21 (m, 4H), 2.34-2.29 (m, 4H), 1.89-1.82 (m, 4H), 1.47 (s, 9H), 1.30-1.25 (m, 6H).

### Step 3: 4,4′-((tert-butoxycarbonyl)azanediyl)dibutanoic acid (4)

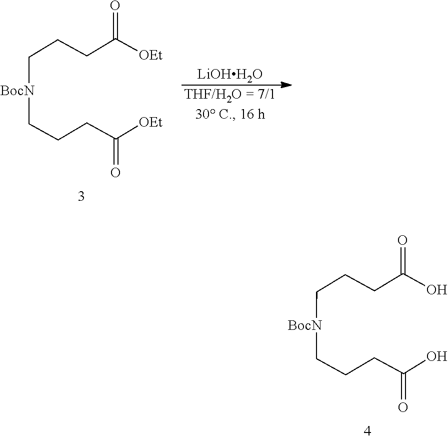 [SMILES: CCOC(=O)CCCN(CCCC(=O)OCC)C(=O)OC(C)(C)C ; CC(C)(C)OC(=O)N(CCCC(=O)O)CCCC(=O)O]

To a solution of ethyl 4-[tert-butoxycarbonyl-(4-ethoxy-4-oxo-butyl)amino]butanoate (12.7 g, 36.77 mmol, 1 eq) in THF (150 mL) was added LiOH·H₂O (5.40 g, 128.68 mmol, 3.5 eq) in H₂O (20 mL). The mixture was stirred at 30° C. for 16 hr. The reaction mixture was diluted with H₂O (120 mL). The aqueous phase was extracted with EtOAc (50 mL*2). Then the aqueous phase was neutralized to pH=4-5 with aq. HCl (1 N) and extracted with EtOAc (150 mL*3). The combined organic phase was washed with brine (120 mL), dried with anhydrous Na₂SO₄, filtered and concentrated in vacuum to give compound 4 (8.5 g, 29.38 mmol, 79.9% yield) as a yellow oil. The crude product was used for next step without further purification.

¹H NMR (400 MHz, CDCl₃) δ=11.88-9.58 (brs, 2H), 3.35-3.15 (m, 4H), 2.37 (t, J=7.2 Hz, 4H), 1.90-1.83 (m, 4H), 1.46 (s, 9H).

### Step 4: di(pentadecan-8-yl) 4,4′-((tert-butoxycarbonyl)azanediyl)dibutanoate (5)

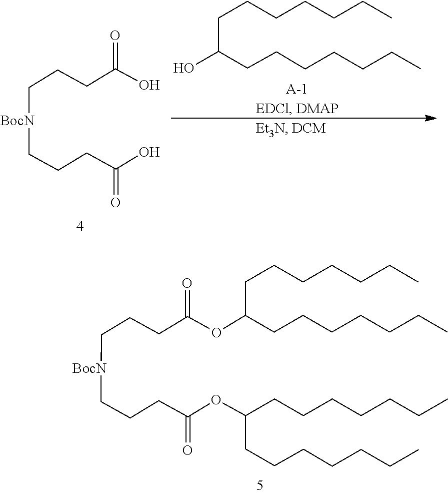

A solution of 4-[tert-butoxycarbonyl(3-carboxypropyl)amino]butanoic acid (2 g, 6.91 mmol, 1.2 eq) dissolved in DCM (30 mL), EDCI (3.31 g, 17.28 mmol, 3 eq), TEA (2.91 g, 28.80 mmol, 4.01 mL, 5 eq) and DMAP (703.8 mg, 5.76 mmol, 1 eq) were added at 0° C. under N₂. After addition, the mixture was stirred at 20° C. for 1 hr, and then pentadecan-8-ol (2.63 g, 11.52 mmol, 2 eq) in DCM (20 mL) was added dropwise. The resulting mixture was stirred at 20° C. for 15 hr. The reaction mixture was diluted with EtOAc (100 mL) and successively washed with saturated aqueous NaHCO₃(50 mL*2), brine (50 mL), dried with anhydrous Na₂SO₄, filtered and concentrated in vacuum to give a residue. The residue was purified by flash silica gel chromatography (80 g SepaFlash® Silica Flash Column, EtOAc/PE: 0˜10%) to give compound 5 (1.5 g, 2.11 mmol, 36.7% yield) as a colorless oil.

¹H NMR (400 MHz, CDCl₃) δ=4.90-4.87 (m, 2H), 3.24-3.21 (m, 4H), 2.30-2.26 (m, 4H), 1.88-1.81 (m, 4H), 1.54-1.18 (m, 8H), 1.46 (s, 9H), 1.34-1.21 (m, 40H), 0.92-0.85 (m, 12H).

### Step 5: di(pentadecan-8-yl) 4,4′-azanediyldibutanoate (A)

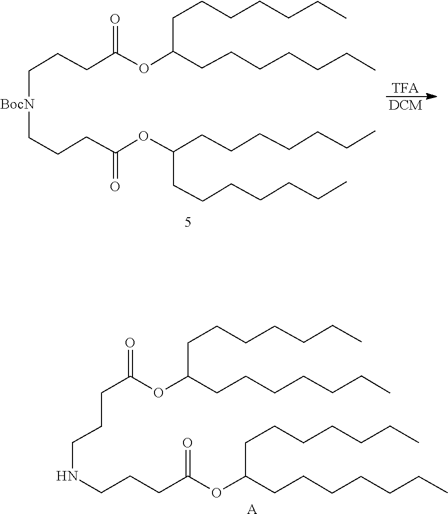

To a solution of 1-heptyloctyl 4-[tert-butoxycarbonyl-[4-(1-heptyloctoxy)-4-oxo-butyl]amino]butanoate (1.3 g, 1.83 mmol, 1 eq) in DCM (20 mL) was added TFA (3.08 g, 27.01 mmol, 2 mL) at 0° C. under N₂. After addition, the mixture was stirred at 20° C. for 4 hr. Then, iced water (20 mL) was added and the mixture was neutralized to pH=8-9 with saturated aqueous NaHCO₃. The aqueous phase was extracted with EtOAc (50 mL*3). The combined organic phase was washed with brine (40 mL), dried with anhydrous Na₂SO₄, filtered and concentrated in vacuum to give compound A (1.06 g, crude) as a yellow oil. The crude product was used for next step without further purification.

¹H NMR (400 MHz, CDCl₃) δ=4.89-4.83 (m, 2H), 2.86-2.82 (m, 4H), 2.42-2.38 (m, 4H), 1.96-1.90 (m, 4H), 1.52-1.50 (m, 8H), 1.32-1.20 (m, 40H), 0.90-0.86 (m, 12H).

Synthesis of intermediate A: Route 2

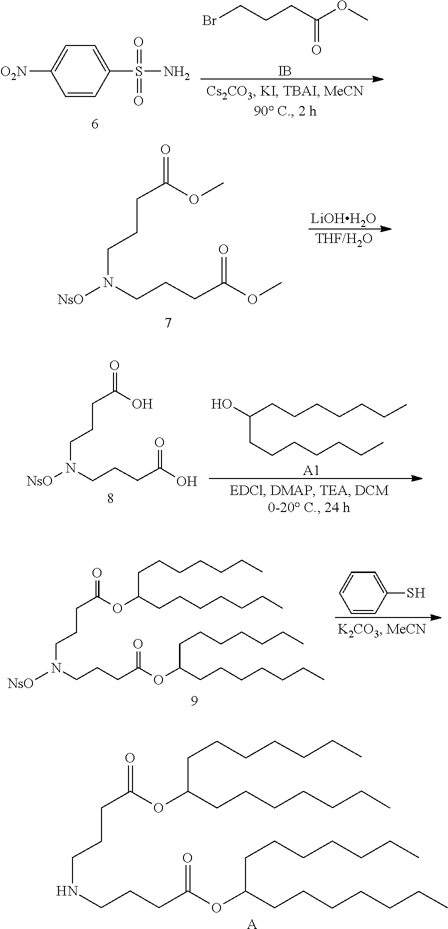

Step 1 dimethyl 4,4′-(((4-nitrophenyl)sulfonyl)azanediyl)dibutanoate (7))

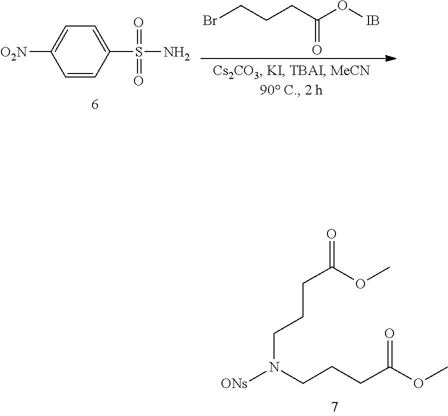

To a solution of methyl 4-bromobutanoate (89.53 g, 494.59 mmol, 4 eq) and 4-nitrobenzenesulfonamide (25 g, 123.65 mmol, 1 eq) in MeCN (500 mL) were added Cs₂CO₃ (80.57 g, 247.30 mmol, 2 eq), KI (10.26 g, 61.82 mmol, 0.5 eq) and TBAI (456.72 mg, 1.24 mmol, 0.01 eq). The mixture was stirred at 90° C. for 12 hours. The reaction mixture was quenched with saturated aqueous NH₄C₁ (1000 mL) and then diluted with EtOAc (500 mL). The aqueous phase was extracted with EtOAc (1000 mL×3). The combined organic phases were washed with brine (600 mL), dried with anhydrous Na₂SO₄, filtered and concentrated in vacuum to give residue, it was purified by silica gel chromatography (PE/EtOAc=10/1 to 3/1) to give compound methyl 4-[(4-methoxy-4-oxo-butyl)-(4-nitrophenyl)sulfonyl-amino]butanoate (48 g, 119.28 mmol, 96.47% yield) as a yellow solid.

¹H NMR (400 MHz, CDCl₃) δ=8.34 (d, J=8.8 Hz, 2H), 7.98 (d, J=8.8 Hz, 2H), 3.67 (s, 6H), 3.21 (t, J=7.6 Hz, 4H), 2.34 (t, J=7.2 Hz, 4H), 1.89-1.82 (m, 4H).

Step 2: 4,4′-(((4-nitrophenyl)sulfonyl)azanediyl)dibutanoic acid (8)

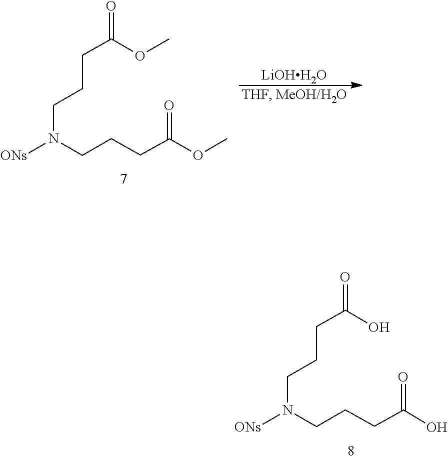

To a solution of methyl 4-[(4-methoxy-4-oxo-butyl)-(4-nitrophenyl)sulfonyl-amino]butanoate (48 g, 119.28 mmol, 1 eq) in THF (300 mL), MeOH (100 mL) and H₂O (100 mL) were added LiOH·H₂O (25.03 g, 596.39 mmol, 5 eq). The mixture was stirred at 25° C. for 12 hours. The reaction mixture was adjusted pH=6 with HCl (2N, aq.), then the solid was filtered and concentrated in vacuum to give compound 4-[3-carboxypropyl-(4-nitrophenyl)sulfonyl-amino]butanoic acid (42 g, 112.19 mmol, 94.06% yield) as a yellow solid.

¹H NMR (400 MHz, DMSO-d₆) δ=8.41-8.36 (m, 2H), 8.10-8.01 (m, 2H), 3.18-3.12 (m, 4H), 2.24-2.18 (m, 4H), 1.75-1.68 (m, 4H).

### Step 3: pentadecan-8-ol (A1)

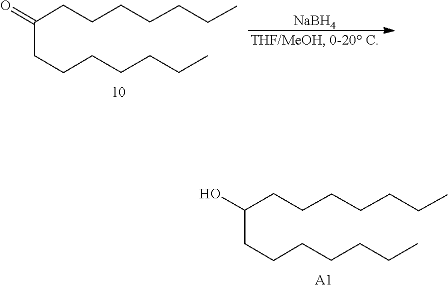

To a solution of pentadecan-8-one (25 g, 110.43 mmol, 1 eq) in THF (300 mL) and MeOH (50 mL) was added NaBH₄ (12.53 g, 331.28 mmol, 3 eq) at 0° C. slowly. The mixture was stirred at 20° C. for 2 hours under N₂. The reaction mixture was quenched with saturated aqueous NH₄Cl (400 mL) and then diluted with EtOAc (500 mL). The aqueous phase was extracted with EtOAc (500 mL×3). The combined organic phases were washed with brine (200 mL), dried with anhydrous Na₂SO₄, filtered and concentrated in vacuum to give residue, it was purified by silica gel chromatography (PE/EtOAc=10/1 to 3/1) to give compound pentadecan-8-ol (23 g, 100.69 mmol, 91.19% yield) as a white solid.

¹H NMR (400 MHz, CDCl₃) δ=3.65-3.56 (m, 1H), 1.55-1.36 (m, 8H), 1.33-1.26 (m, 16H), 0.95-0.82 (m, 6H).

### Step 4: di(pentadecan-8-yl) 4,4′-(((4-nitrophenyl)sulfonyl)azanediyl)dibutanoate (9)

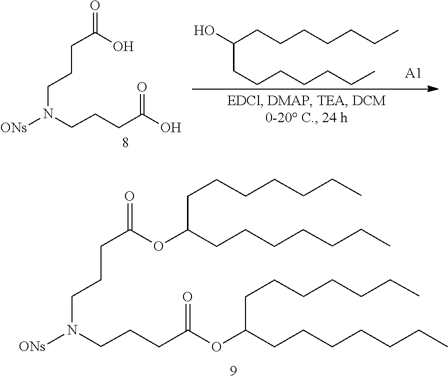

To a solution of 4-[3-carboxypropyl-(4-nitrophenyl)sulfonyl-amino]butanoic acid (12 g, 32.05 mmol, 1 eq) and pentadecan-8-ol (14.64 g, 64.11 mmol, 2 eq) in CH₂Cl₂ (100 mL) were added EDCI (18.43 g, 96.16 mmol, 3 eq), DMAP (3.92 g, 32.05 mmol, 1 eq) and TEA (9.73 g, 96.16 mmol, 13.38 mL, 3 eq). The mixture was stirred at 25° C. for 12 hours. The reaction mixture was quenched by the addition of saturated aqueous NH₄Cl (300 mL) and then extracted with EtOAc (500 mL×3). The combined organic layers were washed with brine (200 mL), dried over sodium sulfate, filtered and concentrated under reduced pressure, it was purified by silica gel chromatography (PE/EtOAc=10/1 to 3/1) to give compound 1-heptyloctyl 4-[[4-(1-heptyloctoxy)-4-oxo-butyl]-(4-nitrophenyl)sulfonyl-amino]butanoate (9 g, 11.32 mmol, 35.31% yield) as a yellow oil.

### Step 5: di(pentadecan-8-yl) 4,4′-azanediyldibutanoate (A): (EC1090-45)

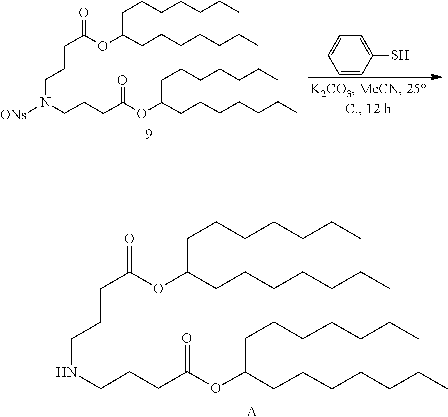

A mixture of 1-heptyloctyl 4-[[4-(1-heptyloctoxy)-4-oxo-butyl]-(4-nitrophenyl)sulfonyl-amino]butanoate (10 g, 12.58 mmol, 1 eq), benzenethiol (1.52 g, 13.83 mmol, 1.41 mL, 1.1 eq), Cs₂CO₃ (8.20 g, 25.15 mmol, 2 eq) in DMF (100 mL) was degassed and purged with N₂ 3 times, and then the mixture was stirred at 25° C. for 12 hours under N₂ atmosphere. The reaction mixture was quenched by the addition of water (500 mL) and then extracted with EtOAc (500 mL×3). The combined organic layers were washed with brine (500 mL), dried over sodium sulfate, filtered and concentrated under reduced pressure. The residue was purified by silica gel chromatography (PE/EtOAc=10/1 to 3/1) to give compound 1-heptyloctyl 4-[[4-(1-heptyloctoxy)-4-oxo-butyl]amino]butanoate (5.6 g, 9.18 mmol, 73.00% yield) as a yellow oil.

¹H NMR (400 MHz, CDCl₃) δ=4.88-4.85 (m, 2H), 2.73-2.70 (m, 4H), 2.38-2.35 (m, 4H), 1.87-1.84 (m, 4H), 1.52-1.50 (m, 8H), 1.32-1.20 (m, 40H), 0.90-0.86 (m, 12H).

### Example 1.1: Synthesis of CAT1

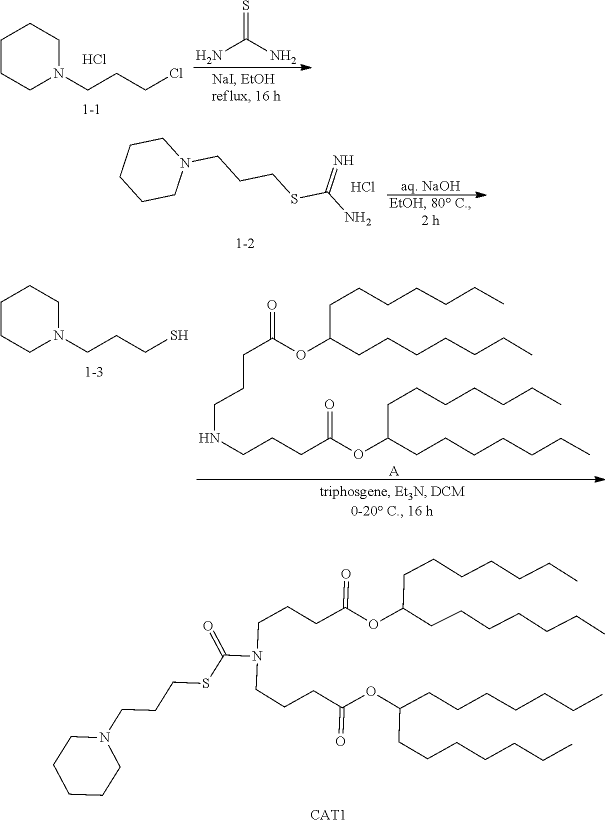

### Step 1: 3-(piperidin-1-yl)propyl carbamimidothioate hydrochloride (1-2)

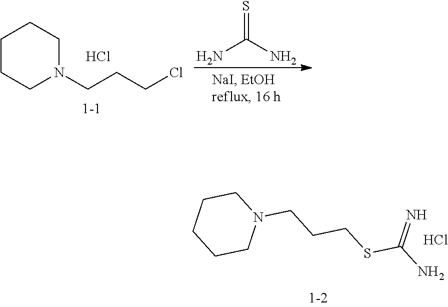

To a solution of 1-(3-chloropropyl)piperidine (10 g, 50.47 mmol, 1 eq, HCl) in EtOH (120 mL) were added NaI (378.3 mg, 2.52 mmol, 0.05 eq) and thiourea (3.84 g, 50.47 mmol, 1 eq). The mixture was stirred at 75° C. for 16 hr. The reaction mixture was cooled to 10° C. and a precipitate formed. The reaction mixture was filtered and the filter cake was washed with EtOAc (30 mL*2). The filter cake and concentrated in vacuum to give compound 1-2 (10.4 g, crude, HCl) as a white solid. The crude product was used for next step without further purification.

### Step 2: 3-(piperidin-1-yl)propane-1-thiol (1-3)

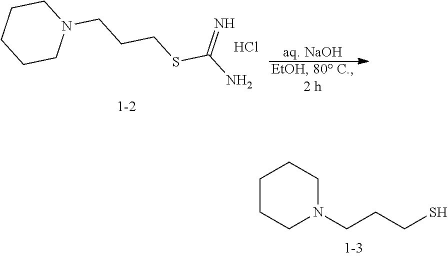 [SMILES: N=C(N)SCCCN1CCCCC1 ; Cl ; SCCCN1CCCCC1]

To a solution of 2-[3-(1-piperidyl)propyl]isothiourea (4 g, 16.82 mmol, 1 eq, HCl) in EtOH (40 mL) was added NaOH (1.01 g, 25.23 mmol, 1.5 eq) in H₂O (5 mL). The mixture was stirred at 80° C. for 2 hr. The reaction mixture was diluted with EtOAc (150 mL). Solid Na₂SO₄ (10 g) was added the reaction mixture. The reaction mixture was filtered and the filter cake was washed with EtOAc (30 mL*2). The filtrate was washed with brine (30 mL*2), dried with anhydrous Na₂SO₄, filtered and concentrated in vacuum to give compound 1-3 (2.1 g, 13.18 mmol, 78.4% yield) as yellow oil. The crude product was used for the next step without further purification.

¹H NMR (400 MHz, CDCl₃) δ=2.71 (t, J=7.6 Hz, 2H), 2.41-2.34 (m, 6H), 1.91-1.84 (m, 2H), 1.60-1.55 (m, 4H), 1.47-1.41 (m, 2H).

### Step 3: di(pentadecan-8-yl) 4,4′-((((3-(piperidin-1-yl)propyl)thio)carbonyl)azanediyl) dibutanoate (CAT1)

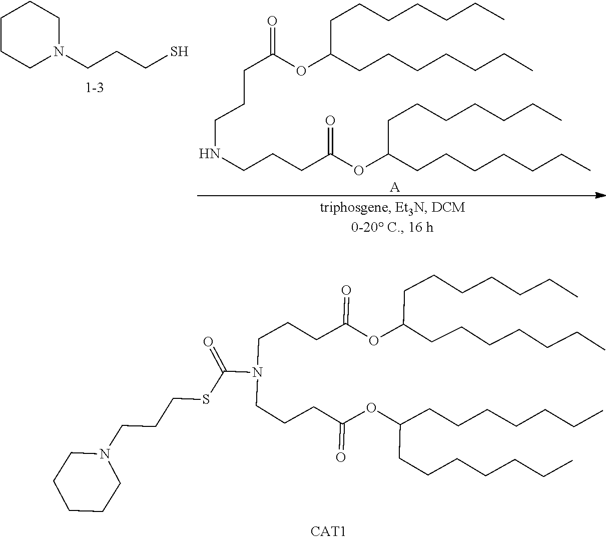

To a solution of 1-heptyloctyl 4-[[4-(1-heptyloctoxy)-4-oxo-butyl]amino]butanoate (700 mg, 1.15 mmol, 1 eq) dissolved in dry DCM (15 mL) were added TEA (348.4 mg, 3.44 mmol, 0.48 mL, 3 eq) and triphosgene (204.3 mg, 0.69 mmol, 0.6 eq) at 0° C. under nitrogen atmosphere. The resulting solution was stirred at 20° C. under nitrogen atmosphere for 1 hour. The resulting reaction mixture was concentrated under reduced pressure and kept under nitrogen atmosphere. NaOH (321.29 mg, 8.03 mmol, 7 eq) was dissolved in dry THF (12 mL) at 0° C., and then 3-(1-piperidyl)propane-1-thiol (913.9 mg, 5.74 mmol, 5 eq) was added under nitrogen atmosphere. To this resulting solution, carbamoyl chloride in THF (10 mL) was added via syringe slowly under nitrogen atmosphere at 0° C. The resulting solution was stirred at 20° C. for 15 hr. The reaction mixture was quenched by NH₄Cl (50 mL) at 0° C. and then diluted with EtOAc (30 mL). The aqueous phase was extracted with EtOAc (40 mL*3). The combined organic phase was washed with brine (50 mL), dried with anhydrous Na₂SO₄, filtered and concentrated in vacuum to give a residue. The residue was purified by flash silica gel chromatography (40 g SepaFlash® Silica Flash Column, DCM: MeOH: 0˜17.5%, 2% NH₃·H₂O in MeOH) to give compound CAT1 (1.02 g, crude) as a yellow oil. Then, the crude product was purified again by flash silica gel chromatography (25 g SepaFlash® Silica Flash Column, PE: EtOAc: 0˜12.5%, 5% NH₃·H₂O in EtOAc) to afford pure compound CAT1 (522 mg, 0.64 mmol, 50.2% yield, 98% purity) as a yellow oil. LCMS: [M+H]⁺: 796.5;

¹H NMR (400 MHz, CDCl₃) δ=4.90-4.84 (m, 2H), 3.38-3.37 (m, 4H), 2.91 (t, J=7.2 Hz, 2H), 2.45-2.22 (m, 10H), 1.94-1.86 (m, 4H), 1.84-1.77 (m, 2H), 1.63-1.47 (m, 12H), 1.46-1.38 (m, 2H), 1.34-1.21 (m, 40H), 0.89 (t, J=7.2 Hz, 12H).

### Example 1.2: Synthesis of CAT6

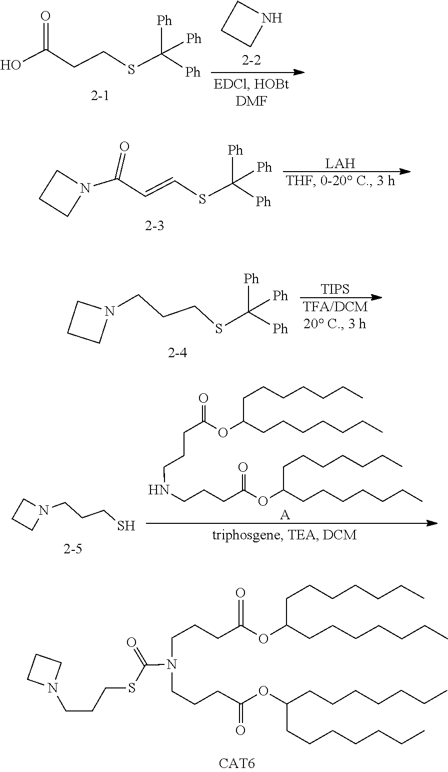

### Step 1: 1-(azetidin-1-yl)-3-(tritylthio)propan-J-one (2-3

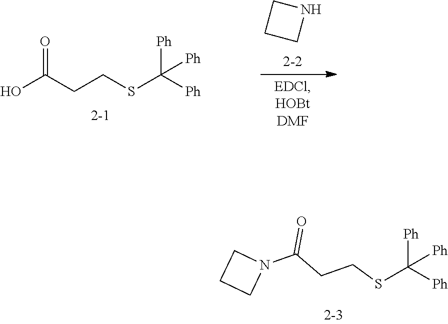

A mixture of 3-tritylsulfanylpropanoic acid (20 g, 57.40 mmol, 1.23 mL, 1 eq), EDCI (16.50 g, 86.09 mmol, 1.5 eq), HOBt (11.63 g, 86.09 mmol, 1.5 eq) in DMF (100 mL) was degassed and purged with N₂ 3 times, and then the mixture was stirred at 20° C. for 1 hr under N₂ atmosphere, and then azetidine (3.93 g, 68.88 mmol, 4.65 mL, 1.2 eq) in DMF (5 mL) was added dropwise at 0° C. The resulting mixture was stirred at 20° C. for 15 hr. After completion, the reaction mixture was diluted with H₂O (150 mL) and extracted with EtOAc (200 mL×3). The combined organic layers were washed with saturated brine (100 mL×2), dried over Na₂SO₄, filtered and concentrated under reduced pressure to give a residue. The residue was purified by flash silica gel chromatography (120 g SepaFlash® Silica Flash Column, EtOAc/PE: 0˜50%) to give compound 2-3 (15.1 g, 38.96 mmol, 67.9% yield) as a white solid. ¹H NMR (400 MHz, CDCl₃) δ=7.45-7.43 (m, 6H), 7.33-7.26 (m, 6H), 7.26-7.19 (m, 3H), 4.03-3.93 (m, 4H), 2.51 (t, J=7.2 Hz, 2H), 2.26-2.18 (m, 2H), 1.98-1.95 (m, 2H).

### Step 2: 1-(3-(tritylthio)propyl)azetidine (2-4)

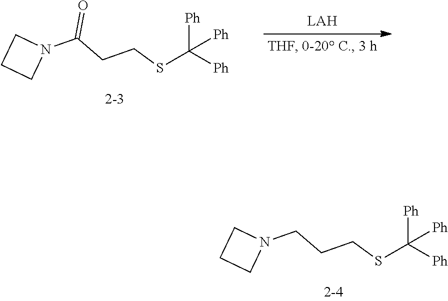 [SMILES: O=C(CCSC(c1ccccc1)(c1ccccc1)c1ccccc1)N1CCC1 ; c1ccc(C(SCCCN2CCC2)(c2ccccc2)c2ccccc2)cc1]

To a solution of 1-(azetidin-1-yl)-3-tritylsulfanyl-propan-1-one (7 g, 18.06 mmol, 1 eq) in THF (120 mL) was added LAH (822.67 mg, 21.68 mmol, 1.2 eq) in portions at 0° C. under N₂. After addition, the resulting mixture was stirred at 20° C. for 3 hr. After completion, the reaction mixture was diluted with THF (60 mL), then successively was added H₂O (0.82 mL), aq.NaOH (0.82 mL, 4M), H₂O (2.5 mL) and Na₂SO₄ (25 g) at 0° C. under N₂. The reaction mixture was filtered and the filtrate was concentrated in vacuum to give crude product. The crude product was triturated with MTBE (50 mL) at 20° C. for 30 min to give compound 2-4 (5.2 g, 13.92 mmol, 77.1% yield) as a light yellow solid.

¹H NMR (400 MHz, DMSO-d₆) δ=7.33-7.29 (m, 12H), 7.26-7.23 (m, 3H), 2.91 (t, J=6.8 Hz, 4H), 2.18 (t, J=6.8 Hz, 2H), 2.10 (t, J=7.6 Hz, 2H), 1.89-1.84 (m, 2H), 1.27-1.22 (m, 2H).

### Step 3: 3-(azetidin-1-yl)propane-1-thiol (2-5)

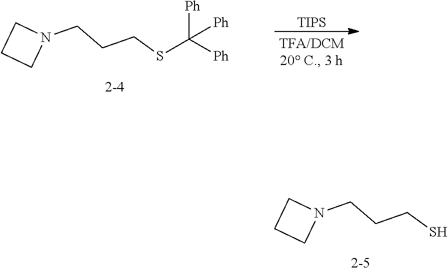 [SMILES: c1ccc(C(SCCCN2CCC2)(c2ccccc2)c2ccccc2)cc1 ; SCCCN1CCC1 ; "TIPS"]

To a solution of 1-(3-tritylsulfanylpropyl)azetidine (4 g, 10.71 mmol, 1 eq) in DCM (30 mL) were added TFA (23.10 g, 202.59 mmol, 15 mL, 18.92 eq) and TIPS (4.20 g, 21.42 mmol, 2 eq) at 0° C. under N₂. After addition, the resulting mixture was stirred at 20° C. for 3 hr. After completion, the reaction mixture was concentrated under reduced pressure to remove TFA. The residue was diluted with MeOH (100 mL) and extracted with PE (50 mL×5). The MeOH layers was concentrated under reduced pressure to give compound 2-5 (2.4 g, crude, TFA) as a yellow oil.

¹H NMR (400 MHz, DMSO-d₆) δ=4.12-4.09 (m, 2H), 3.99-3.97 (m, 2H), 3.22-3.17 (m, 2H), 2.51-2.50 (m, 2H), 2.40-2.38 (m, 1H), 2.32-2.22 (m, 1H), 1.74-1.70 (m, 2H).

### Step 4: di(pentadecan-8-yl) 4,4′-((((3-(azetidin-1-yl)propyl)thio)carbonyl)azanediyl)dibutanoate (CAT6)

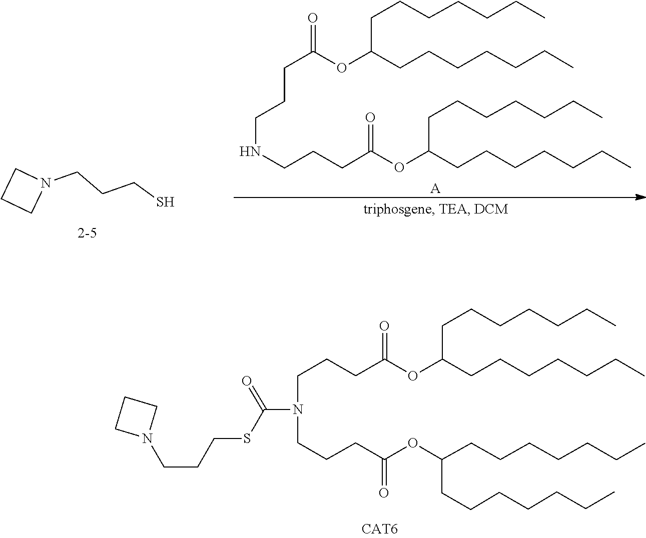

To a solution of 1-heptyloctyl 4-[[4-(1-heptyloctoxy)-4-oxo-butyl]amino]butanoate (1.50 g, 2.46 mmol, 1 eq) dissolved in dry dichlormethane (30.0 mL) were added triethylamine (746.48 mg, 7.38 mmol, 1.03 mL, 3 eq) and triphosgene (437.83 mg, 1.48 mmol, 0.6 eq) at 0° C. under nitrogen atmosphere. The resulting solution was stirred at 20° C. for 1 hr. After that, the resulting reaction was concentrated under reduced pressure. At the same time, to a solution of 3-(azetidin-1-yl)propane-1-thiol (2.11 g, 8.61 mmol, 3.5 eq, TFA) dissolved in dry tetrahydrofuran (30.0 mL) was added NaOH (688.52 mg, 17.22 mmol, 7 eq) at 0° C. under nitrogen atmosphere. Then carbamoyl chloride, which was dissolved in tetrahydrofuran (15 mL), was added to this resulting solution via syringe slowly at 0° C. under nitrogen atmosphere. After that, the resulting solution was stirred at 20° C. for 15 hrs under nitrogen atmosphere. After completion, the reaction mixture was quenched by NH₄Cl (60 mL) at 0° C. and then diluted with EtOAc (50 mL). The aqueous phase was extracted with EtOAc (60 mL*3). The combined organic phase was washed with brine (50 mL), dried with anhydrous Na₂SO₄, filtered and concentrated in vacuum to give residue. The residue was purified by prep-HPLC (column: Welch Ultimate XB-SiOH 250*50*10 um; mobile phase: [Hexane-EtOH];B %: 0%-30%, 10 min) to give compound CAT6 (322 mg, 419.69 umol, 49.54% yield, 100% purity) as a light yellow oil.

### LCMS [M+1]⁺: 767.5;

¹H NMR (400 MHz, CDCl₃) δ=4.90-4.84 (m, 2H), 3.42-3.31 (m, 4H), 3.19 (t, J=6.8 Hz, 4H), 2.90 (t, J=7.2 Hz, 2H), 2.47 (t, J=8.0 Hz, 2H), 2.36-2.26 (m, 4H), 2.08-2.05 (m, 2H), 1.95-1.85 (m, 4H), 1.67-1.65 (m, 2H), 1.52-1.50 (m, 8H), 1.30-1.26 (m, 40H), 0.90-0.86 (m, 12H).

### Example 1.3: Synthesis of CAT7

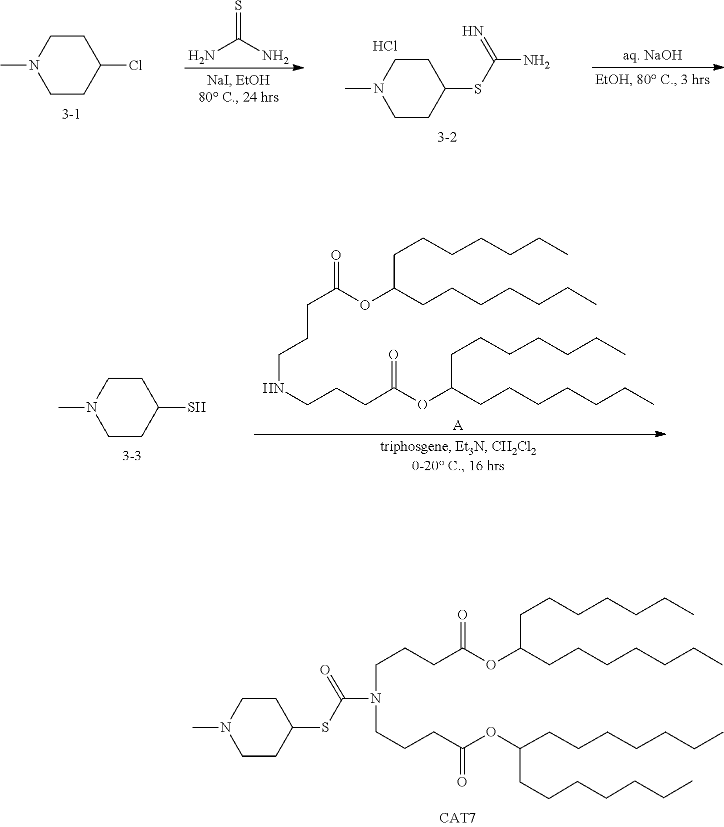

### Step 1: I-methylpiperidin-4-yl carbamimidothioate (3-2)

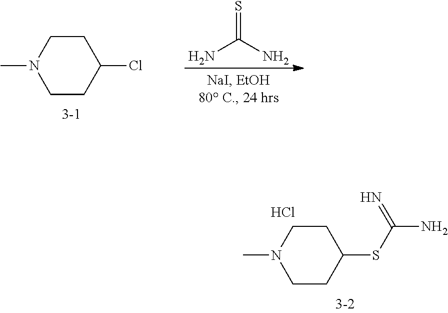

To a solution of 4-chloro-1-methylpiperidine (20.0 g, 150 mmol, 1.00 eq.) and thiourea (28.5 g, 74.2 mmol, 2.50 eq.) in ethanol (100 mL) was added sodium iodide (2.24 g, 15.0 mmol, 0.10 eq.). The mixture was degassed and purged with nitrogen three times, then the mixture was stirred at 80° C. for 24 hours under nitrogen atmosphere to give compound 3-2 (60.0 g, crude, hydrochloric acid salt) as a yellow gum.

¹H NMR (400 MHz, CDCl₃) δ=3.06-3.02 (m, 1H), 2.70 (s, 3H), 2.67-2.54 (m, 4H), 1.91-1.73 (m, 4H)

### Step 2: 1-methylpiperidine-4-thiol (3-3)

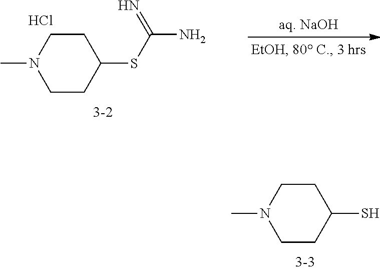

To a solution of 1-methylpiperidin-4-yl carbamimidothioate (16.0 g, 76.3 mmol, 1.00 eq., hydrochloric acid salt) in ethanol (80.0 mL) was added sodium hydroxide (18.3 g, 458 mmol, 6.00 eq.) which dissolved in water (10.0 mL). The mixture was degassed and purged with nitrogen three times, and then the mixture was stirred at 80° C. for 3 hours under nitrogen atmosphere. After completion, the mixture was concentrated and then extracted with ethyl acetate (200 mL×3). The combined organic layers were dried with anhydrous sodium sulfate and filtered. The filtrate was concentrated under reduced pressure to compound 3-3 (4.20 g, crude) as a yellow gum.

### Step 3: di(pentadecan-8-yl) 4,4′-((((1-methylpiperidin-4-yl)thio)carbonyl)azanediyl)dibutanoate (CAT7)

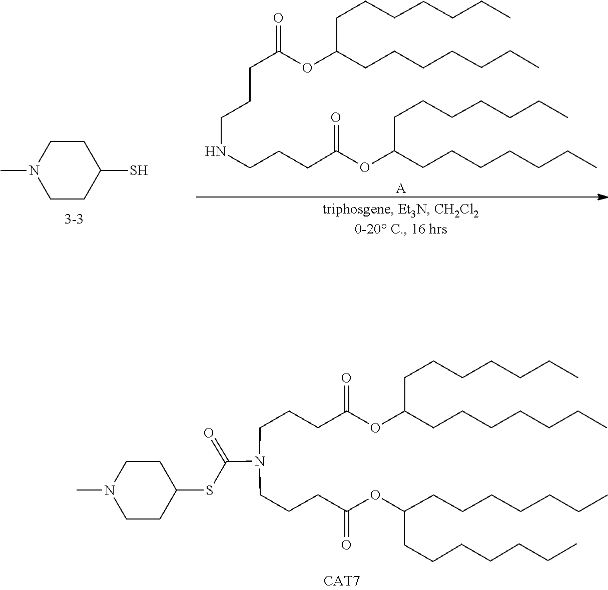

To a solution of di(pentadecan-8-yl) 4,4′-azanediyldibutanoate (2.00 g, 3.28 mmol, 1.00 eq.) dissolved in dry dichloromethane (30.0 mL) were added triethylamine (995 mg, 9.84 mmol, 1.37 mL, 3.00 eq.) and triphosgene (584 mg, 1.97 mmol, 0.60 eq.) at 0° C. under nitrogen atmosphere. The resulting solution was stirred at 20° C. for 1 hour. After that, the resulting reaction was concentrated under reduced pressure. At the same time, to a solution of 1-methylpiperidine-4-thiol (2.15 g, 16.4 mmol, 5.00 eq.) dissolved in dry tetrahydrofuran (20.0 mL) was added sodium hydroxide (918 mg, 23.0 mmol, 7.00 eq.) at 0° C. under nitrogen atmosphere. Finally, carbamoyl chloride, which was dissolved in tetrahydrofuran (20.0 mL), was added to this resulting solution via syringe slowly at 0° C. under nitrogen atmosphere. The resulting solution was stirred at 20° C. for 15 hours under nitrogen atmosphere. After completion, the mixture was quenched by saturated ammonium chloride aqueous solution (200 mL) at 0° C. and then extracted with ethyl acetate (200 mL×3), dried with anhydrous sodium sulfate and filtered. The filtrate was concentrated under reduced pressure. The residue was purified by column chromatography (silica gel, petroleum ether/ethyl acetate/NH₃·H₂O=50/I/0.05 to 2/I/0.05) and prep-HPLC (neutral condition; column: Welch Ultimate XB-CN 250*50*10 μm; mobile phase: [Hexane-EtOH]; B %: 0%-10%, 12 min) to give -CAT7 (350 mg, 452 umol, 51.7% yield, 99.6% purity) as a yellow oil.

### LCMS [M+1]⁺: 768.4;

¹H NMR (400 MHz, CDCl₃) δ=4.91-4.84 (m, 2H), 3.46-3.33 (m, 4H), 2.93-2.81 (m, 2H), 2.36 (s, 3H), 2.34-2.28 (m, 5H), 2.14-2.04 (m, 2H), 1.93-1.79 (m, 6H), 1.55-1.49 (m, 8H), 1.31-1.24 (m, 42H), 0.91-0.86 (m, 12H).

### Example 1.4: Synthesis of CAT8

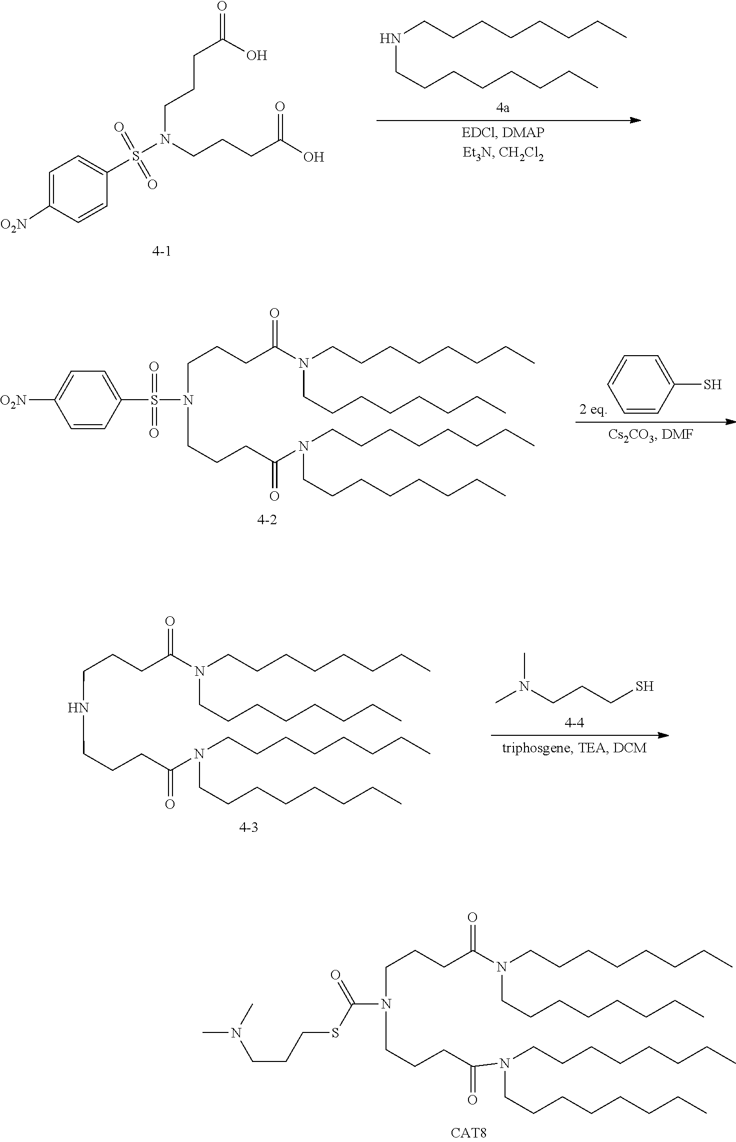

### Step 1: 4,4′-(((4-nitrophenyl)sulfonyl)azanediyl)bis(N,N-dioctylbutanamide) (4-2)

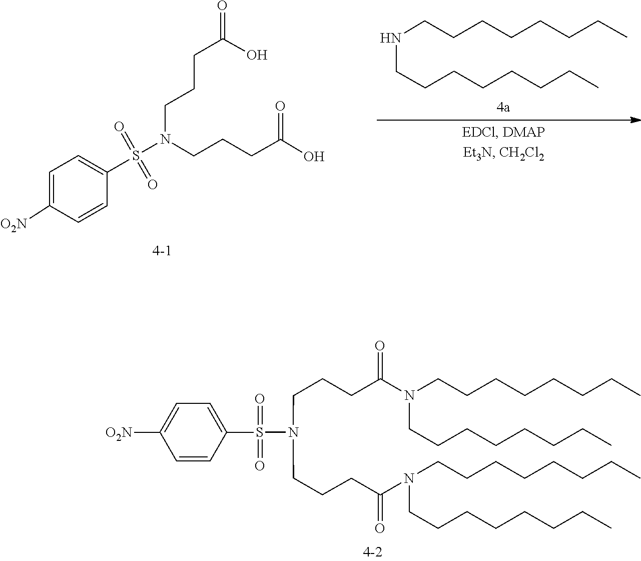 [SMILES: O=C(O)CCCN(CCCC(=O)O)S(=O)(=O)c1ccc([N+](=O)[O-])cc1 ; CCCCCCCCNCCCCCCCC ; CCCCCCCCN(CCCCCCCC)C(=O)CCCN(CCCC(=O)N(CCCCCCCC)CCCCCCCC)S(=O)(=O)c1ccc([N+](=O)[O-])cc1]

To a solution of 4-[3-carboxypropyl-(4-nitrophenyl)sulfonyl-amino]butanoic acid (6.00 g, 16.0 mmol, 1 eq) in DCM (50 mL) were added EDCI (9.22 g, 48.1 mmol, 3 eq), TEA (4.87 g, 48.1 mmol, 6.69 mL, 3 eq) and DMAP (979 mg, 8.01 mmol, 0.5 eq) at 0° C. under N₂. After addition, the mixture was stirred at 20° C. for 1 hour, and then a solution of N-octyloctan-1-amine (8.13 g, 33.7 mmol, 2.1 eq) in DCM (10 mL) was added to dropwise. The resulting mixture was stirred at 20° C. for 6 hours. The reaction mixture was quenched by the addition of water (100 mL), and then extracted with ethyl acetate (200 mL×3). The combined organic layers were washed with brine (100 mL), dried over sodium sulfate, filtered and concentrated under reduced pressure. The residue was purified by prep-HPLC (column: Welch Ultimate XB-CN 250*50*10 um; mobile phase: [Hexane-EtOH]; B %: 0%-15%, 12 min) to yield compound 4-2 (9.00 g, 11.0 mmol, 68% yield) as a yellow oil.

### LCMS: [M+H]⁺: 821.6.

### Step 2: 4,4′-azanediylbis(N,N-dioctylbutanamide) (4-3)

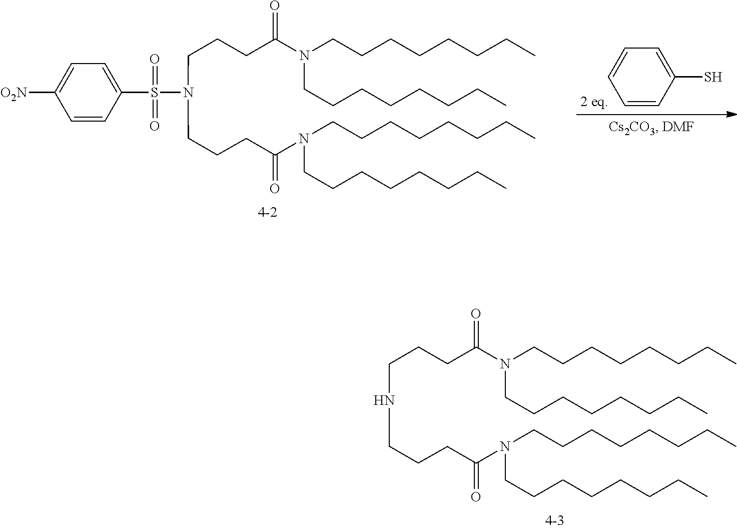 [SMILES: CCCCCCCCN(CCCCCCCC)C(=O)CCCN(CCCC(=O)N(CCCCCCCC)CCCCCCCC)S(=O)(=O)c1ccc([N+](=O)[O-])cc1 ; Sc1ccccc1 ; CCCCCCCCN(CCCCCCCC)C(=O)CCCNCCCC(=O)N(CCCCCCCC)CCCCCCCC]

To a solution of 4-[[4-(dioctylamino)-4-oxo-butyl]-(4-nitrophenyl)sulfonyl-amino]-N,N-dioctyl-butanamide (8.00 g, 9.74 mmol, 1 eq) and benzenethiol (2.15 g, 19.5 mmol, 1.99 mL, 2 eq) in DMF (100 mL) was added Cs₂CO₃ (6.35 g, 19.5 mmol, 2.0 eq). The mixture was stirred at 20° C. for 12 hours under N₂. The reaction mixture was quenched by the addition of water (100 mL), and then extracted with ethyl acetate (300 mL×3). The combined organic layers were washed with brine (500 mL), dried over sodium sulfate, filtered and concentrated under reduced pressure. The residue was purified by prep-HPLC (column: Welch Ultimate XB-CN 250*50*10 μm; mobile phase: [Hexane-EtOH]; B %: 5%-50%, 30 min) to yield compound 4-3 (2.90 g, 4.56 mmol, 47% yield) as a yellow oil.

### LCMS: [M+H]⁺: 637.4.

### Step 3: S-(3-(dimethylamino)propyl) bis(4-(dioctylamino)-4-oxobutyl)carbamothioate (CAT8)

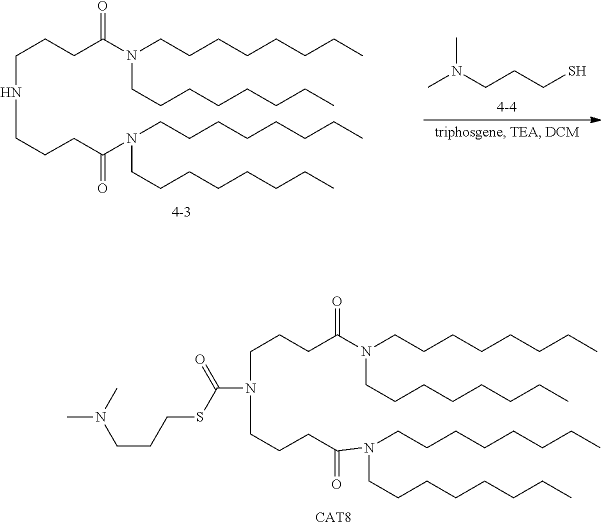

To a solution of 4-[[4-(dioctylamino)-4-oxo-butyl]amino]-N,N-dioctyl-butanamide (2.00 3.14 mmol, 1 eq) dissolved in dry DCM (20 mL) were added TEA (955 mg, 9.43 mmol, 1.31 mL, 3 eq) and bis(trichloromethyl) carbonate (467 mg, 1.57 mmol, 0.5 eq) at 0° C. under N₂. The resulting solution was stirred at 20° C. for 1 hour. The resulting reaction was concentrated under reduced pressure and kept under N₂. To a solution of 3-(dimethylamino)propane-1-thiol (1.87 g, 15.7 mmol, 5 eq) in dry THF (20 mL) was added NaOH (880 mg, 22.0 mmol, 7 eq) at 0° C. under N₂. To this resulting solution, carbamoyl chloride was added via syringe slowly under N₂ at 0° C. The resulting solution was stirred at 20° C. for 15 hours. The reaction mixture was quenched with saturated aqueous NH₄C₁ (100 mL) and then diluted with ethyl acetate (100 mL). The aqueous phase was extracted with ethyl acetate (100 mL×3). The combined organic phase was washed with brine (100 mL), dried with anhydrous Na₂SO₄, filtered and concentrated under vacuum to give residue. The residue was purified by column chromatography (SiO₂, Dichloromethane/Methanol=50/1 to 10/1). Compound S-[3-(dimethylamino)propyl] N,N-bis[4-(dioctylamino)-4-oxo-butyl]carbamothioate (4.10 g, crude) was obtained as a yellow oil.

### LCMS: [M+H]⁺: 756.1;

¹H NMR (400 MHz, CDCl₃) δ: 4.82-4.77 (m, 2H), 3.39-3.29 (m, 4H), 2.84 (t, J=7.2 Hz, 2H), 2.31-2.22 (m, 6H), 2.17-2.15 (m, 6H), 1.85-1.70 (m, 6H), 1.46-1.42 (m, 8H), 1.25-1.10 (m, 40H), 0.86-0.72 (m, 12H).

### Example 1.5: Synthesis of CAT3

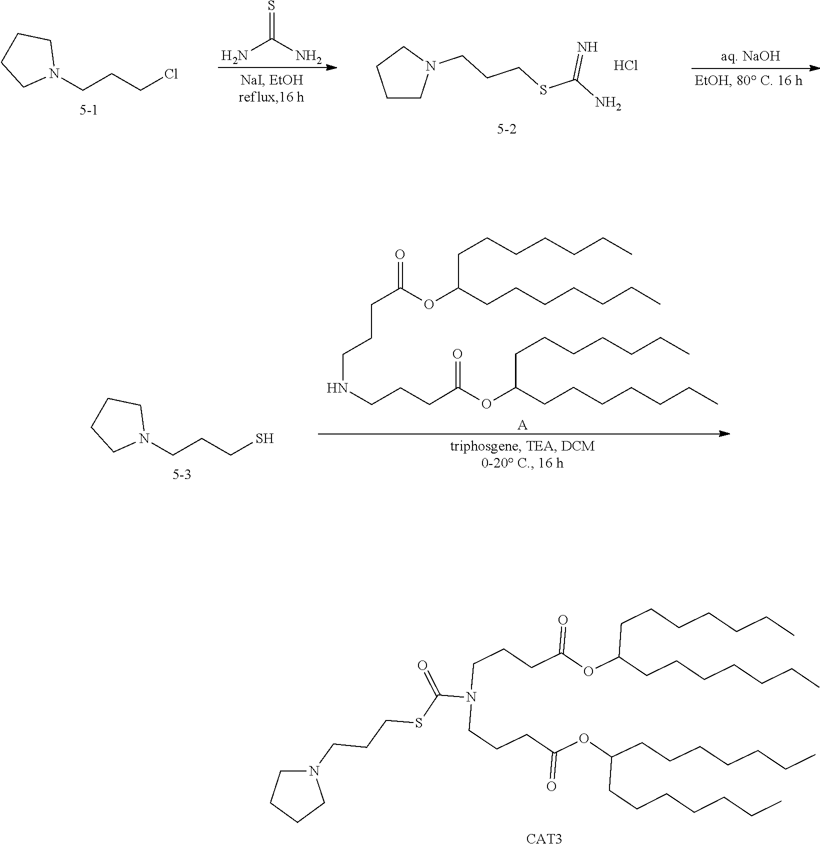

### Step 1: 3-(pyrrolidin-1-yl)propyl carbamimidothioate hydrochloride (5-2)

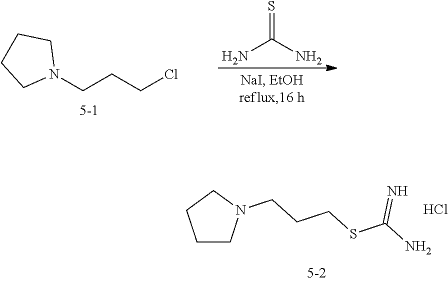

To a solution of 1-(3-chloropropyl)pyrrolidine (25 g, 169.32 mmol, 1 eq, HCl) in EtOH (300 mL) were added NaI (1.27 g, 8.47 mmol, 0.05 eq) and thiourea (13.53 g, 177.79 mmol, 1.05 eq). The mixture was stirred at 75° C. for 16 hr. The reaction mixture was cooled to 0° C. and precipitate formed. The reaction mixture was filtered and the filter cake was washed with EtOAc (50 mL*3). The filter cake was concentrated in vacuum to give compound 5-2 (22.5 g, 100.55 mmol, 59.4% yield, HCl) as a white solid. The crude product was used for next step without further purification.

¹H NMR (400 MHz, DMSO-d₆) δ=11.24 (s, 1H), 9.37 (s, 3H), 3.52-3.44 (m, 2H), 3.33-3.31 (m, 2H), 3.22-3.14 (m, 2H), 3.02-2.92 (m, 2H), 2.09-2.00 (m, 2H), 2.00-1.92 (m, 2H), 1.91-1.82 (m, 2H).

### Step 2: 3-(pyrrolidin-1-yl)propane-1-thiol (5-3)

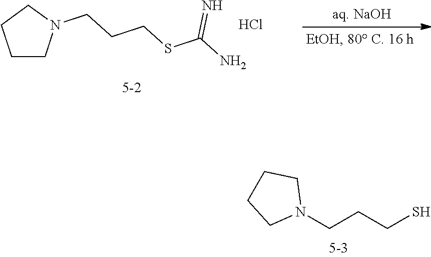 [SMILES: N=C(N)SCCCN1CCCC1 ; Cl ; SCCCN1CCCC1]

To a solution of 2-(3-pyrrolidin-1-ylpropyl)isothiourea (5.2 g, 23.24 mmol, 1 eq, HCl) in EtOH (80 mL) was added NaOH (2.79 g, 69.72 mmol, 3 eq) in H₂O (10 mL). The mixture was stirred at 80° C. for 16 hr. The reaction mixture was diluted with EtOAc (150 mL). Then, the mixture was washed with brine (30 mL*2), dried with anhydrous Na₂SO₄, filtered, and concentrated in vacuum to give compound 5-3 (2.8 g, 19.28 mmol, 82.9% yield) as a yellow oil. The crude product was used for next step without further purification.

¹H NMR (400 MHz, CDCl₃) δ=2.73 (t, J=7.2 Hz, 1H), 2.62-2.53 (m, 2H), 2.50-2.48 (m, 6H), 1.83-1.75 (m, 6H).

### Step 3: di(pentadecan-8-yl) 4,4′-((((3-(pyrrolidin-1-yl)propyl)thio)carbonyl) azanediyl)dibutanoate (CAT3)

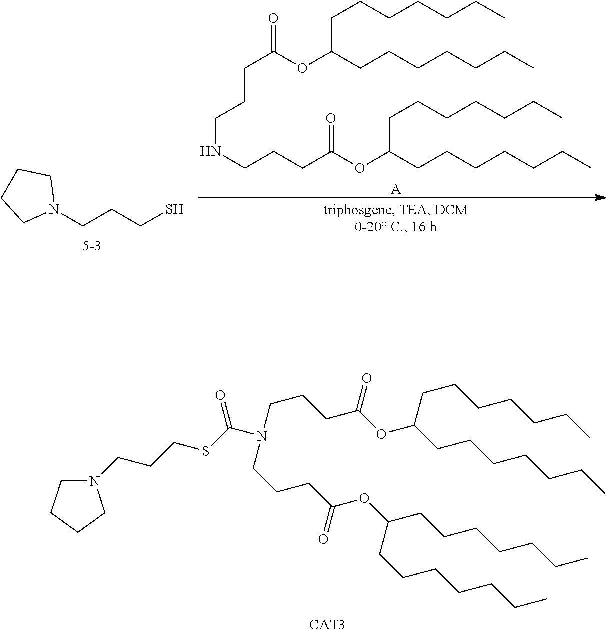

To a solution of 1-heptyloctyl 4-[[4-(1-heptyloctoxy)-4-oxo-butyl]amino]butanoate (1.2 g, 1.97 mmol, 1 eq) in dry DCM (15 mL) were added TEA (597.2 mg, 5.90 mmol, 0.82 mL, 3 eq) and triphosgene (350.3 mg, 1.18 mmol, 0.6 eq) at 0° C. under nitrogen atmosphere. The resulting solution was stirred at 20° C. under nitrogen atmosphere for 1 hour. The resulting reaction mixture was concentrated under reduced pressure and kept under nitrogen atmosphere. To a solution of 3-pyrrolidin-1-ylpropane-1-thiol (1.00 g, 6.89 mmol, 3.5 eq) in dry THF (12 mL) at 0° C. under nitrogen atmosphere was added NaOH (550.8 mg, 13.77 mmol, 7 eq) under nitrogen atmosphere. A solution of carbamoyl chloride in THF (10 mL) was added via syringe slowly under nitrogen atmosphere at 0° C. to the resulting solution which was stirred at 200 C for 15 hr. The reaction mixture was quenched by NH₄Cl (60 mL) at 0° C. and then diluted with EtOAc (40 mL). The aqueous phase was extracted with EtOAc (50 mL*3). The combined organic phase was washed with brine (50 mL), dried with anhydrous Na₂SO₄, filtered, and concentrated in vacuum to give a residue. The residue was purified by flash silica gel chromatography (40 g SepaFlash® Silica Flash Column, PE: EtOAc: 0˜15%, 5% NH₃·H₂O in EtOAc) to give compound CAT3 (1.1 g, crude) as a yellow oil. Then the crude product was purified again by flash silica gel chromatography (25 g SepaFlash® Silica Flash Column, EtOAc: PE: 0˜12%, 5% NH₃·H₂O in EtOAc) to afford pure compound CAT3 (395 mg, 0.50 mmol, 30.0% yield, 98.7% purity) as a yellow oil.

### LCMS: [M+H]⁺: 781.6;

¹H NMR (400 MHz, CDCl₃) δ=4.90-4.84 (m, 2H), 3.39-3.37 (m, 4H), 2.94 (t, J=7.2 Hz, 2H), 2.56-2.44 (m, 6H), 2.33-2.28 (m, 4H), 1.97-1.81 (m, 6H), 1.80-1.74 (m, 4H), 1.56-1.46 (m, 8H), 1.35-1.24 (m, 40H), 0.88 (t, J=7.2 Hz, 12H).

### Example 1.6: Synthesis of CAT4

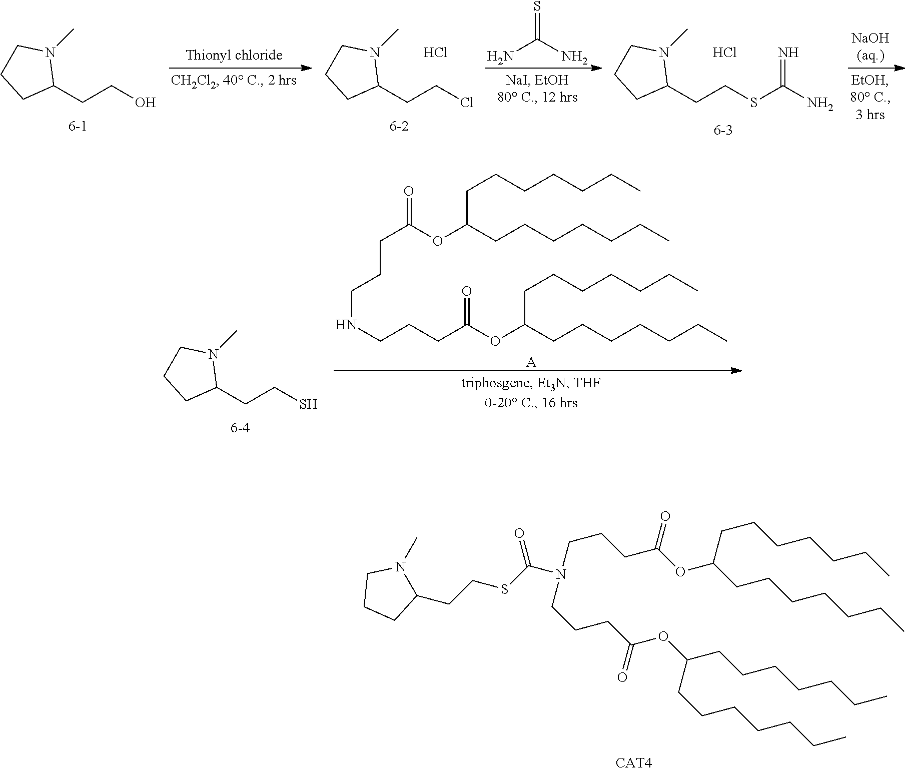

### Step 1: 2-(2-chloroethyl)-1-methylpyrrolidine hydrochloride (6-2)

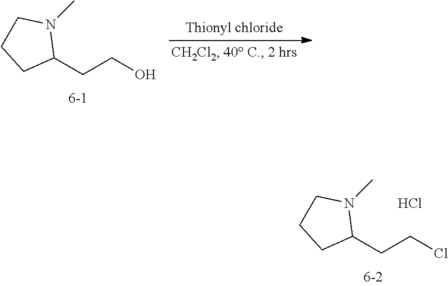

To a solution of 2-(1-methylpyrrolidin-2-yl)ethanol (2.00 g, 15.5 mmol, 2.10 mL, 1.00 eq.) in dichlormethane (20.0 mL) was added thionyl chloride (5.52 g, 46.4 mmol, 3.37 mL, 3.00 eq.) drop-wise. Then, the mixture was stirred at 40° C. for 2 hours. After completion, the reaction mixture was filtered and concentrated under reduced pressure to give compound 6-2 (2.20 g, crude) as a yellow solid.

¹H NMR (400 MHz, DMSO-d₆) δ=11.32 (s, 1H), 3.86-3.79 (m, 1H), 3.71-3.63 (m, 1H), 3.53-3.44 (m, 1H), 3.40-3.29 (m, 1H), 3.06-2.96 (m, 1H), 2.74 (d, J=4.8 Hz, 3H), 2.40-2.31 (m, 1H), 2.26-2.10 (m, 2H), 2.02-1.83 (m, 2H), 1.74-1.63 (m, 1H).

### Step 2: 2-(1-methylpyrrolidin-2-yl)ethyl carbamimidothioate hydrochloride (6-3)

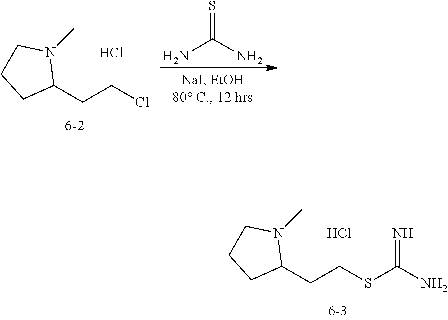

A mixture of 2-(2-chloroethyl)-1-methylpyrrolidine hydrochloride (14.0 g, 76.0 mmol, 1.00 eq.), thiourea (5.90 g, 77.6 mmol, 1.02 eq.) and sodium iodide (2.28 g, 15.2 mmol, 0.20 eq.) in ethanol (100 mL) was degassed and purged with nitrogen three times, then the mixture was stirred at 80° C. for 12 hours under nitrogen atmosphere. After completion, the reaction mixture was cooled down to ambient temperature. Then ethyl acetate (100 mL) was added until permanent opalescence was detected and the mixture was maintained at 4° C. for 12 hours. After that, the mixture was filtered and concentrated under reduced pressure to compound 6-3 (16.0 g, 71.5 mmol, 94.0% yield) as a yellow solid.

¹H NMR (400 MHz, DMSO-d₆) δ=10.99 (s, 1H), 9.32 (s, 3H), 3.50-3.39 (m, 2H), 3.35-3.29 (m, 2H), 3.08-2.98 (m, 1H), 2.77 (d, J=4.8 Hz, 3H), 2.28-2.16 (m, 2H), 2.04-1.93 (m, 2H), 1.92-1.69 (m, 2H).

### Step 3: 2-(1-methylpyrrolidin-2-yl)ethanethiol (6-4)

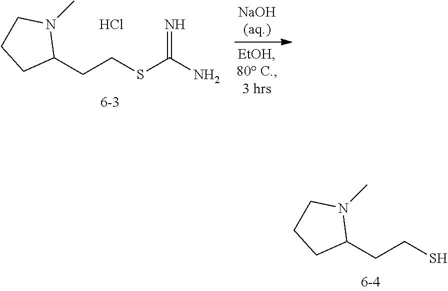

To a solution of 2-(1-methylpyrrolidin-2-yl)ethyl carbamimidothioate hydrochloride (10.0 g, 44.7 mmol, 1.00 eq.) in ethanol (80.0 mL) was added sodium hydroxide (5.36 g, 134 mmol, 3.00 eq.) which dissolved in water (20.0 mL). The mixture was stirred at 80° C. for 3 hours under nitrogen atmosphere. After completion, the mixture was concentrated and then extracted with ethyl acetate (200 mL×3). The combined organic layers were dried with anhydrous sodium sulfate and filtered. The filtrate was concentrated under reduced pressure to compound 6-4 (2.40 g, crude) as a yellow oil.

¹H NMR (400 MHz, CDCl₃) δ=3.08-3.01 (m, 2H), 2.50-2.45 (m, 1H), 2.31 (s, 3H), 2.18-2.09 (m, 4H), 1.79-1.65 (m, 4H).

### Step 4: di(pentadecan-8-yl) 4,4′-((((2-(I-methylpyrrolidin-2-yl)ethyl)thio)carbonyl)azanediyl)dibutanoate (CAT4)

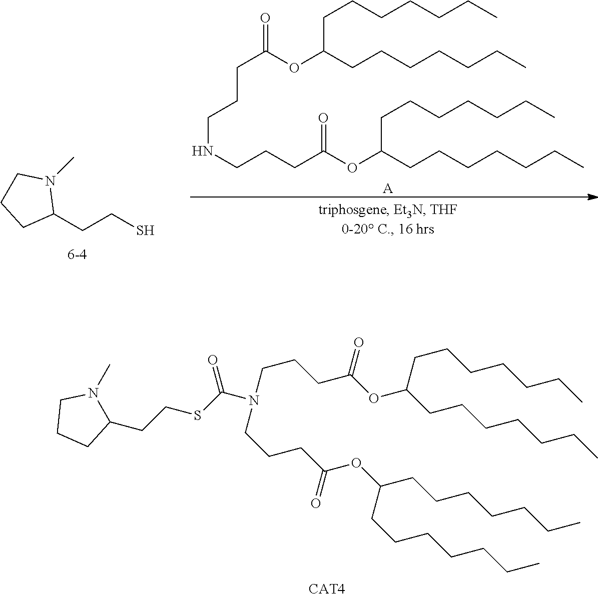

To a solution of di(pentadecan-8-yl) 4,4′-azanediyldibutanoate (2.00 g, 3.28 mmol, 1.00 eq.) dissolved in dry dichlormethane (30.0 mL) were added triethylamine (995 mg, 9.84 mmol, 1.37 mL, 3.00 eq.) and triphosgene (584 mg, 1.97 mmol, 0.60 eq.) at 0° C. under nitrogen atmosphere. The resulting solution was stirred at 20° C. for 1 hour. After that, the resulting reaction was concentrated under reduced pressure. At the same time, to a solution of 2-(1-methylpyrrolidin-2-yl)ethanethiol (2.38 g, 16.4 mmol, 5.00 eq.) dissolved in dry tetrahydrofuran (20.0 mL) was added sodium hydroxide (918 mg, 22.9 mmol, 7.00 eq.) at 0° C. under nitrogen atmosphere. Then, carbamoyl chloride, which was dissolved in tetrahydrofuran (20.0 mL), was added to this resulting solution via syringe slowly at 0° C. under nitrogen atmosphere. After that, the resulting solution was stirred at 20° C. for 15 hours under nitrogen atmosphere. After completion, the mixture was quenched by ammonium chloride (200 mL) at 0° C. and then extracted with ethyl acetate (200 mL×3), dried with anhydrous sodium sulfate and filtered. The filtrate was concentrated under reduced pressure. The residue was purified by column chromatography (silica gel, petroleum ether/ethyl acetate/NH₃·H₂O=I/O/0.05 to 10/I/O. 05) to give CAT4 (451 mg, 576 umol, 17.6% yield, 99.9% purity) as a yellow oil.

### LCMS [M+1]⁺: 781.5;

¹H NMR (400 MHz, CDCl₃) δ=4.91-4.84 (m, 2H), 3.43-3.33 (m, 4H), 3.14-3.03 (m, 1H), 3.00-2.92 (m, 1H), 2.89-2.80 (m, 1H), 2.33 (s, 3H), 2.32-2.29 (m, 2H), 2.23-2.09 (m, 2H), 2.03-1.86 (m, 6H), 1.80-1.67 (m, 2H), 1.58-1.47 (m, 1OH), 1.33-1.22 (m, 42H), 0.92-0.85 (m, 12H).

### Example 1.7: Synthesis of 23 (CAT4)

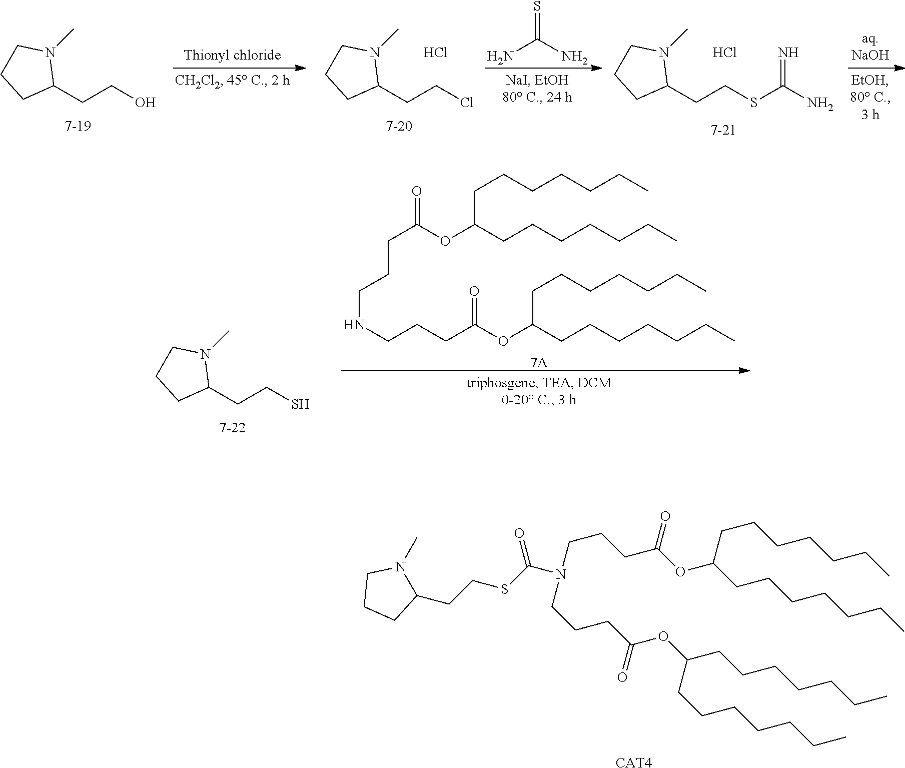

### Step 1: 2-(2-chloroethyl)-1-methylpyrrolidine hydrochloride (20)

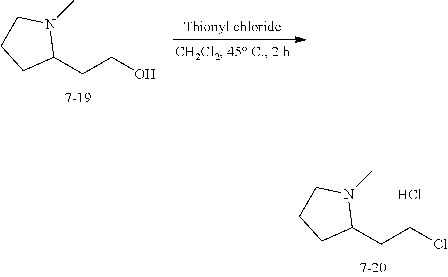

To a solution of 2-(1-methylpyrrolidin-2-yl)ethanol (2 g, 15.48 mmol, 2.10 mL, 1 eq) in CH₂Cl₂ (20 mL) was added SOCl₂ (5.52 g, 46.44 mmol, 3.37 mL, 3 eq) dropwised slowly. The mixture was stirred at 45° C. for 2 hours. The reaction mixture was filtered and concentrated under reduced pressure to give compound 2-(2-chloroethyl)-1-methyl-pyrrolidine (2.2 g, 11.95 mmol, 77.19% yield, hydrochloride salt) as a yellow solid.

¹H NMR (400 MHz, DMSO-d₆) δ=11.32 (s, 1H), 3.88-3.80 (m, 1H), 3.75-3.66 (m, 1H), 3.55-3.45 (m, 1H), 3.43-3.35 (m, 1H),. 3.08-2.97 (m, 1H), 2.75 (s, 3H), 2.48-2.31 (m, 1H), 2.28-2.10 (m, 2H), 2.04-1.88 (m, 2H), 1.82-1.66 ppm (m, 1H).

### Step 2: 2-(1-methylpyrrolidin-2-yl)ethyl carbamimidothioate hydrochloride (21)

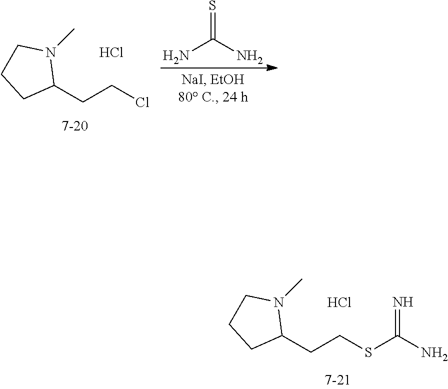

A mixture of 2-(2-chloroethyl)-1-methyl-pyrrolidine (14 g, 76.04 mmol, 1 eq, hydrochloride salt), thiourea (5.90 g, 77.56 mmol, 1.02 eq), NaI (2.28 g, 15.21 mmol, 0.2 eq) in EtOH (100 mL) was degassed and purged with N₂ 3 times, and then the mixture was stirred at 80° C. for 12 hours under N₂ atmosphere. The reaction mixture was cooled to ambient temperature. EtOAc (100 mL) until permanent opalescence was obtained. Then the reaction mixture was stood at 4° C. for 12 hours. The mixture was then filtered and concentrated under reduced pressure to give compound 2-[2-(1-methylpyrrolidin-2-yl)ethyl]isothiourea (16 g, 71.50 mmol, 94.03% yield, hydrochloride salt) as a yellow solid.

### LCMS: [M+H]⁺: 188.1.

### Step 3: 2-(I-methylpyrrolidin-2-yl)ethanethiol (22)

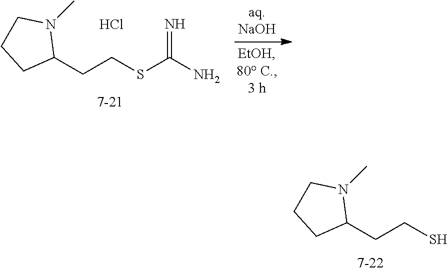

To a solution of 2-[2-(1-methylpyrrolidin-2-yl)ethyl]isothiourea (3 g, 13.41 mmol, 1 eq, hydrochloride salt) in H₂O (1 mL) and EtOH (8 mL) was added NaOH (2.68 g, 67.03 mmol, 5 eq). The mixture was stirred at 90° C. for 2 hours. The mixture was filtered and concentrated under reduced pressure to give compound 2-(1-methylpyrrolidin-2-yl)ethanethiol (1.8 g, 12.39 mmol, 92.42% yield) as a yellow oil which was used next step without purification.

### Step 4: di(pentadecan-8-yl) 4,4′-((((2-(1-methylpyrrolidin-2-yl)ethyl)thio)carbonyl)azanediyl) dibutanoate (23)

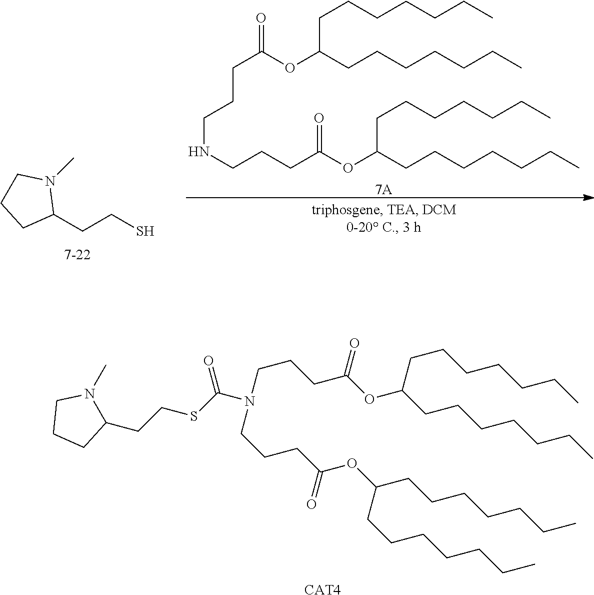

To a solution of 1-heptyloctyl 4-[[4-(1-heptyloctoxy)-4-oxo-butyl]amino]butanoate (1.5 g, 2.46 mmol, 1 eq) dissolved in dry CH₂Cl₂ (15 mL) were added TEA (746.47 mg, 7.38 mmol, 1.03 mL, 3 eq) and triphosgen (364.85 mg, 1.23 mmol, 0.5 eq) at 0° C. under nitrogen atmosphere. The resulting solution was stirred at 20° C. under nitrogen atmosphere for 1 hour. The reaction was concentrated under reduced pressure and kept under nitrogen atmosphere. NaOH (688.47 mg, 17.22 mmol, 7 eq) was dissolved in dry THF (20 mL) at 0° C. under nitrogen atmosphere, then 2-(1-methylpyrrolidin-2-yl)ethanethiol (1.79 g, 12.30 mmol, 5 eq) was added under nitrogen atmosphere. To this resulting solution, carbamoyl chloride dissolved in THF (10 mL) was added slowly under nitrogen atmosphere at 0° C. The mixture was stirred at 20° C. for 12 hours. The reaction mixture was quenched by water (50 mL) and then diluted with EtOAc (50 mL), then extracted with EtOAc (50 mL×3). The combined organic phase was washed with brine (20 mL), dried with anhydrous Na₂SO₄, filtered and concentrated in vacuum to give residue. The residue was purified by silica gel chromatography (PE/EtOAc=20/1 to 0/1, 6% NH₃·H₂O in EtOAc) to give compound CAT4 (551 mg, 695.39 umol, 28.27% yield, 98.6% purity) as a yellow oil.

### LCMS: [M+H]⁺: 781.9;

¹H NMR (400 MHz, CDCl₃) δ=4.95-4.78 (m, 2H), 3.51-3.40 (m, 4H), 3.12-3.03 (m, 1H), 3.01-2.94 (m, 1H), 2.91-2.83 (m, 1H), 2.32 (s, 3H), 2.31-2.26 (m, 2H), 2.22-2.10 (m, 2H), 2.04-1.94 (m, 6H), 1.85-1.62 (m, 4H), 1.59-1.52 (m, 8H), 1.37-1.18 (m, 42H), 0.88 (t, J=6.8 Hz, 12H).

### Example 1.8: Synthesis of CAT5

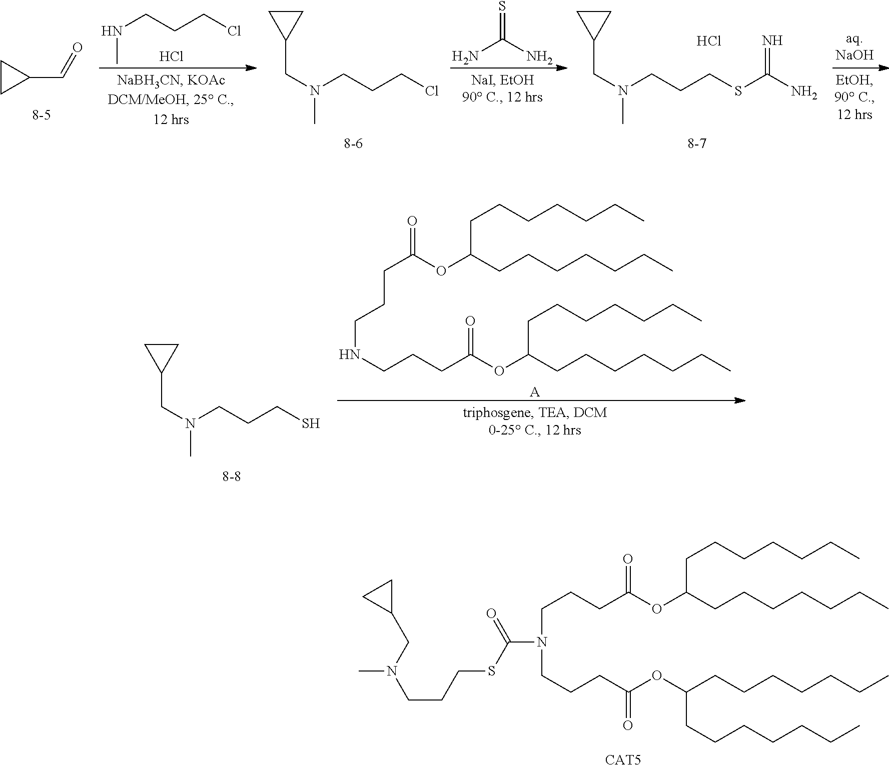

### Step 1: 3-chloro-N-(cyclopropylmethyl)-N-methyl-propan-1-amine (8-6)

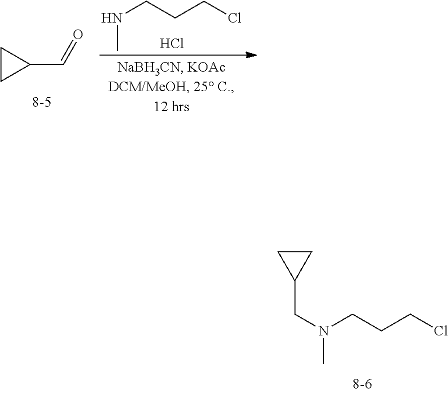

To a solution of cyclopropanecarbaldehyde (19.46 g, 277.70 mmol, 20.75 mL, 2 eq) and 3-chloro-N-methyl-propan-1-amine (20 g, 138.85 mmol, 1 eq, hydrochloride) in dichlormethane (200 mL) were added NaBH₃CN (13.09 g, 208.27 mmol, 1.5 eq) and KOAc (40.88 g, 416.54 mmol, 3 eq). The mixture was stirred at 25° C. for 12 hours. The reaction mixture was quenched with saturated aqueous NH₄C₁ (500 mL) and then diluted with ethyl acetate (300 mL). The aqueous phase was extracted with ethyl acetate (500 mL×3). The combined organic phase was washed with brine (100 mL), dried with anhydrous Na₂SO₄, filtered and concentrated in vacuum to give residue. The residue was purified by silica gel chromatography (petroleum ether/ethyl acetate=10/1 to 3/1 and ethyl acetate/methanol=30/1 to 10/1) to give compound 8-6 (15 g, 92.78 mmol, 66.82% yield) as a yellow oil.

¹H NMR (400 MHz, CDCl₃) δ=3.62-3.55 (m, 2H), 2.54-2.51 (m, 2H), 2.28-2.23 (m, 5H), 2.02-1.97 (m, 2H), 0.90-0.85 (m, 1H), 0.55-0.48 (m, 2H), 0.18-0.08 (m, 2H).

### Step 2: 2-[3-[cyclopropylmethyl(methyl)amino]propyl]isothiourea hydrochloride (8-7)

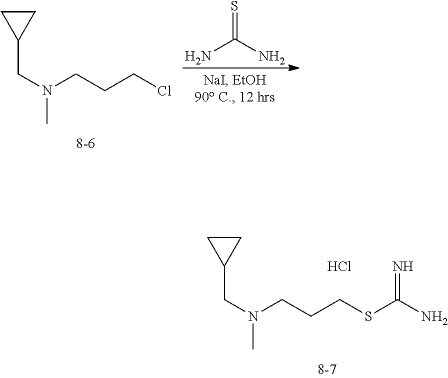

To a solution of 3-chloro-N-(cyclopropylmethyl)-N-methyl-propan-1-amine (7 g, 43.30 mmol, 1 eq) and thiourea (3.96 g, 51.96 mmol, 1.2 eq) in ethanol (15 mL) was added NaI (649.01 mg, 4.33 mmol, 0.1 eq). The mixture was stirred at 90° C. for 12 hours. The reaction mixture was filtered and concentrated under reduced pressure to give compound 8-7 (8 g, 33.64 mmol, 77.70% yield, hydrochloride) as a brown oil.

¹H NMR (400 MHz, DMSO-d₆) (5 7.03-6.95 (m, 4H), 3.28-3.24 (m, 1H), 2.91-2.85 (m, 2H), 2.70-2.66 (m, 2H), 2.53-2.48 (m, 3H), 2.38-2.32 (m, 2H), 1.72-1.58 (m, 2H), 0.98-0.90 (m, 1H), 0.48-0.41 (m, 2H), 0.26-0.12 (m, 2H).

### Step 3: 3-[cyclopropylmethyl(methyl)amino]propane-1-thiol (8-8)

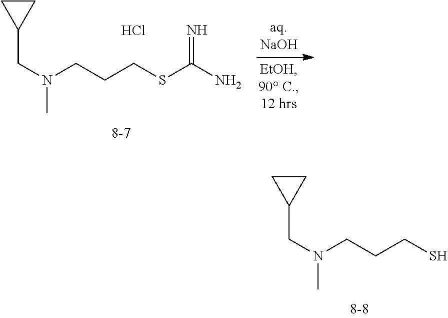

To a solution of 2-[3-[cyclopropylmethyl(methyl)amino]propyl]isothiourea (8 g, 39.74 mmol, 1 eq hydrochloride) in ethanol (16 mL) and water (4 mL) was added NaOH (9.54 g, 238.41 mmol, 6 eq). The mixture was stirred at 90° C. for 12 hours. The reaction mixture was filtered and concentrated under reduced pressure to give compound 8-8 (2.4 g, 15.07 mmol, 37.92% yield) as a yellow oil.

### Step 4: 1-heptyloctyl 4-[3-[cyclopropylmethyl(methyl)amino]propylsulfanylcarbonyl-[4-(]-heptyloctoxy)-4-oxo-butyl]amino]butanoate (CAT5)

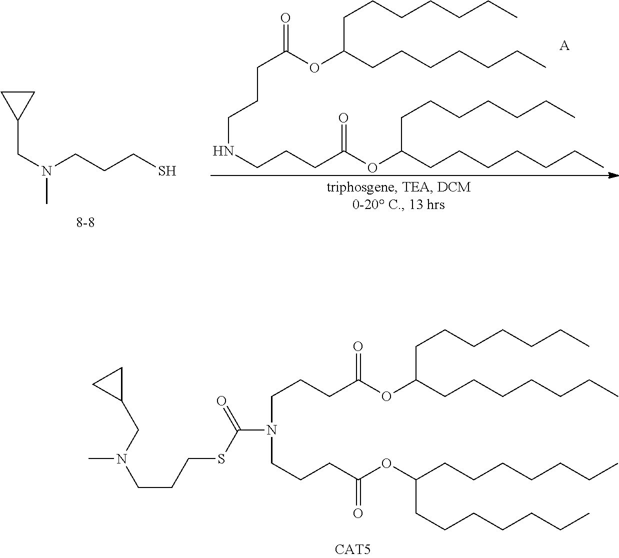

To a solution of 1-heptyloctyl 4-[[4-(1-heptyloctoxy)-4-oxo-butyl]amino]butanoate (2 g, 3.28 mmol, 1 eq) dissolved in dry dichlormethane (20 mL) were added TEA (995.30 mg, 9.84 mmol, 1.37 mL, 3 eq) and triphosgene (486.47 mg, 1.64 mmol, 0.5 eq) at 0° C. under nitrogen atmosphere. The resulting solution was stirred at 20° C. under nitrogen atmosphere for 1 hour. The reaction was concentrated under reduced pressure and kept under nitrogen atmosphere. NaOH (917.96 mg, 22.95 mmol, 7 eq) was dissolved in dry THF (20 mL) at 0° C. under nitrogen atmosphere, then 3-[cyclopropylmethyl(methyl)amino]propane-1-thiol (2.61 g, 16.39 mmol, 5 eq) was added under nitrogen atmosphere. To this resulting solution, carbamoyl chloride dissolved in THF (10 mL) was added slowly under nitrogen atmosphere at 0° C. The mixture was stirred at 200 C for 12 hours. The reaction mixture was quenched with saturated aqueous NH₄C₁ (100 mL) and then diluted with ethyl acetate (100 mL). The aqueous phase was extracted with ethyl acetate (100 mL×3). The combined organic phase was washed with brine (100 mL), dried with anhydrous Na₂SO₄, filtered and concentrated in vacuum to give residue. The residue was purified by silica gel chromatography (petroleum ether/ethyl acetate=10/1 to 1/1 and dichloromethane/methanol=30/1 to 10/1) to give compound CAT5 (1.0 g, 1.26 mmol, 38.31% yield, 99.9% purity) as a yellow oil.

### LCMS: [M+H]⁺: 796.2;

¹H NMR (400 MHz, CDCl₃) δ=4.87-4.85 (m, 2H), 3.49-3.35 (m, 4H), 2.92 (t, J=7.2 Hz, 2H), 2.47 (t, J=7.2 Hz, 2H), 2.42-2.30 (m, 7H), 2.24 (d, J=6.4 Hz, 2H), 1.98-1.94 (m, 4H), 1.80-1.74 (m, 2H), 1.53-1.48 (m, 8H), 1.28-1.20 (m, 40H), 0.98-0.90 (m, 13H), 0.51 (d, J=8.0 Hz, 2H), 0.11-0.010 (m, 2H).

### Example 1.9: Synthesis of CAT9

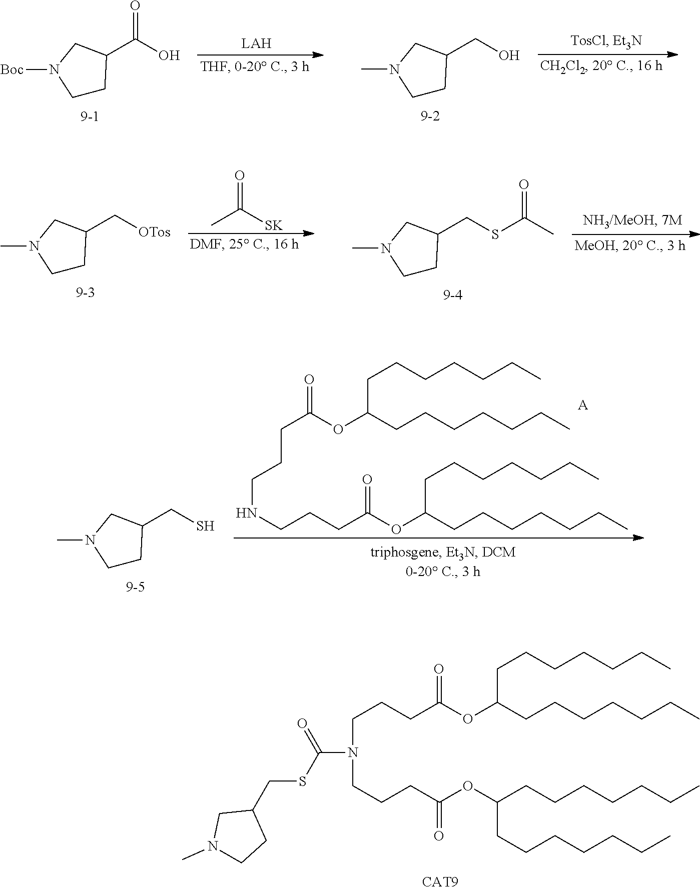

### Step 1: (1-methylpyrrolidin-3-yl)methanol (9-2)

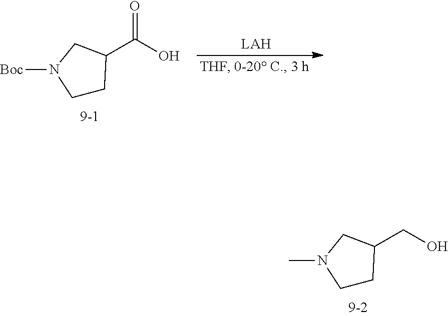

To a solution of 1-tert-butoxycarbonylpyrrolidine-3-carboxylic acid (30 g, 139.38 mmol, 1 eq) in THF (600 mL) was added LAH (15.87 g, 418.13 mmol, 3 eq) in portions at 0° C. under N₂. After addition, the mixture was stirred at 20° C. for 3 hr. After completion, the reaction mixture was diluted with THF (350 mL), then successively was added H₂O (16 mL), aq.NaOH (16 mL, 4M), H₂O (20 mL) and Na₂SO₄ (100 g) at 0° C. under N₂. The reaction mixture was filtered and the filtrate was concentrated in vacuum to give compound 9-2 (11.2 g, 97.25 mmol, 69.8% yield) as a yellow oil.

¹H NMR (400 MHz, CDCl₃) δ=3.67-3.63 (m, 1H), 3.54-3.50 (m, 1H), 2.95-2.68 (m, 2H), 2.58-2.52 (m, 1H), 2.51-2.44 (m, 1H), 2.40-2.33 (m, 1H), 2.32 (s, 3H), 2.02-1.97 (m, 1H), 1.66-1.63 (m, 1H).

### Step 2: (J-methylpyrrolidin-3-yl)methyl 4-methylbenzenesulfonate (9-3)

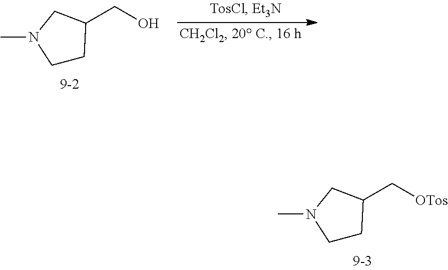

To a solution of (1-methylpyrrolidin-3-yl)methanol (10 g, 86.83 mmol, 1 eq) in DCM (200 mL) were added TEA (17.57 g, 173.65 mmol, 24.17 mL, 2 eq), DMAP (1.06 g, 8.68 mmol, 0.1 eq) and TosCl (19.86 g, 104.19 mmol, 1.2 eq) at 0° C. under N₂. The mixture was stirred at 20° C. for 16 hr. After completion, the reaction mixture was diluted with DCM (150 mL) and washed with brine (100 mL*2), dried with anhydrous Na₂SO₄, filtered and concentrated in vacuum to give residue. The residue was purified by flash silica gel chromatography (120 g SepaFlash® Silica Flash Column, Methanol: Dichloromethane 0˜15%) to give compound 9-3 (10.8 g, 40.10 mmol, 46.2% yield) as a yellow oil.

¹H NMR (400 MHz, CDCl₃) δ=7.79 (d, J=8.4 Hz, 2H), 7.35 (d, J=8.0 Hz, 2H), 3.93 (d, J=7.2 Hz, 2H), 2.60-2.46 (m, 4H), 2.45 (s, 3H), 2.31 (s, 3H), 2.30-2.28 (m, 1H), 1.97-1.95 (m, 1H), 1.45-1.31 (m, 1H).

### Step 3: S-((1-methylpyrrolidin-3-yl)methyl) ethanethioate (9-4)

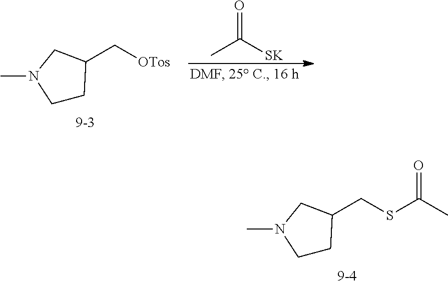

To a solution of (1-methylpyrrolidin-3-yl)methyl 4-methylbenzenesulfonate (10.7 g, 39.72 mmol, 1 eq) in DMF (100 mL) was added acetylsulfanylpotassium (5.44 g, 47.67 mmol, 1.2 eq) under N₂. The mixture was stirred at 25° C. for 16 hr. After completion, The reaction mixture was cooled to 0° C. and quenched by the addition of H₂O (150 mL). Then, the reaction was diluted with EtOAc (100 mL) and extracted with EtOAc (150 mL*3). The combined organic phase was washed with brine (150 mL), dried with anhydrous Na₂SO₄, filtered and concentrated in vacuum. The residue was purified by flash silica gel chromatography (40 g SepaFlash® Silica Flash Column, Dichloromethane: Methanol: 0˜10%) to give compound 9-4 (4.8 g, 27.70 mmol, 69.7% yield) as a yellow oil.

¹H NMR (400 MHz, CDCl₃) δ=2.97-2.94 (m, 2H), 2.72-2.71 (m, 1H), 2.59-2.51 (m, 2H), 2.45-2.41 (m, 1H), 2.34-2.33 (m, 6H), 2.24-2.22 (m, 1H), 2.21-2.03 (m, 1H), 1.52-1.48 (m, 1H).

### Step 4: (1-methylpyrrolidin-3-yl)methanethiol (9-5)

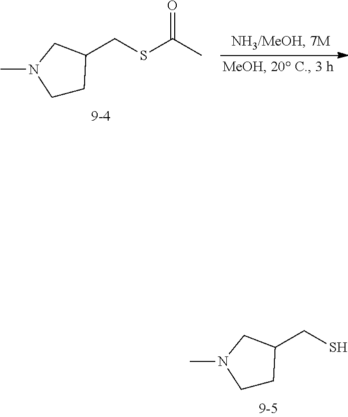

To a solution of S-[(1-methylpyrrolidin-3-yl)methyl] ethanethioate (1.7 g, 9.81 mmol, 1 eq) in MeOH (10 mL) was added NH₃ (7 M in MeOH, 4.20 mL, 3 eq). The mixture was stirred at 20° C. for 3 hr under N₂. After completion, the reaction mixture was concentrated under reduced pressure (air bath, water pump) to remove solvent to give compound 9-5 (1.2 g, crude) as a yellow oil. The crude product was used in the next step without further purification.

¹H NMR (400 MHz, CD30D) δ=2.86-2.81 (m, 1H), 2.69-2.64 (m, 1H), 2.59-2.56 (m, 3H), 2.48-2.41 (m, 1H), 2.38 (s, 3H), 2.34-2.32 (m, 1H), 2.11-2.07 (m, 1H), 1.60-1.57 (m, 1H).

### Step 5: di(pentadecan-8-yl) 4,4′-(((((1-methylpyrrolidin-3-yl)methyl)thio)carbonyl)azanediyl)dibutanoate (CAT9)

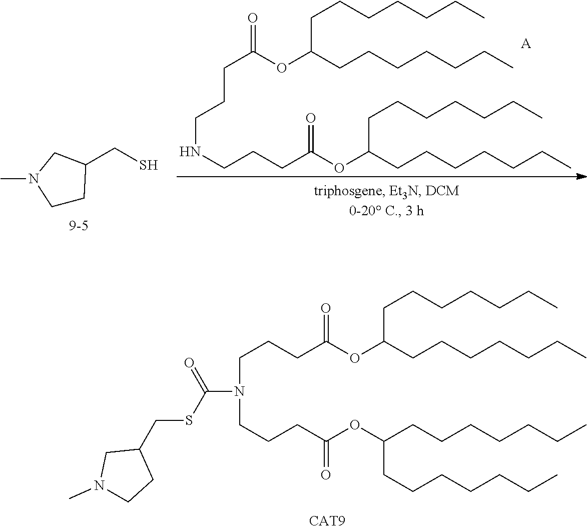

To a solution of 1-heptyloctyl 4-[[4-(1-heptyloctoxy)-4-oxo-butyl]amino]butanoate (1.5 g, 2.46 mmol, 1 eq) dissolved in dry DCM (25 mL) were added TEA (746.48 mg, 7.38 mmol, 1.03 mL, 3 eq) and triphosgene (437.83 mg, 1.48 mmol, 0.6 eq) at 0° C. under N₂. The resulting solution was stirred at 20° C. for 1 hour. The resulting reaction was concentrated under reduced pressure and kept under N₂. To a solution of (1-methylpyrrolidin-3-yl)methanethiol (1.13 g, 8.61 mmol, 3.5 eq) dissolved in dry THF (30 mL) was added NaOH (688.52 mg, 17.21 mmol, 7 eq) at 0° C. under N₂. To this resulting solution, carbamoyl chloride dissolved in THF (25 mL) was added via syringe slowly under N₂ at 0° C. The resulting solution was stirred at 20° C. for 2 hr. After completion, the reaction mixture was quenched by NH₄Cl (60 mL) at 0° C. and then diluted with EtOAc (50 mL). The aqueous phase was extracted with EtOAc (50 mL*3). The combined organic phase was washed with brine (60 mL), dried with anhydrous Na₂SO₄, filtered and concentrated in vacuum to give residue. The residue was purified by flash silica gel chromatography (20 g SepaFlash® Silica Flash Column, Ethyl acetate: Petroleum ether: 0˜13%, 5% NH₃·H₂O in Ethyl acetate) to give 620 mg compound, and then the compound was purified by prep-HPLC (column: Welch Ultimate XB-SiOH 250*50*10 um; mobile phase: [Hexane-EtOH];B %: 0%-30%, 13 min) to give compound CAT9 (325 mg, 0.42 mmol, 17.9% yield, 99.10% purity) as a light yellow oil.

### LCMS [M+1]⁺: 767.9;

¹H NMR (400 MHz, CDCl₃) δ=4.91-4.84 (m, 2H), 3.41-3.36 (m, 4H), 3.05-2.98 (m, 2H), 2.81-2.77 (m, 1H), 2.63-2.59 (m, 2H), 2.51-2.46 (m, 1H), 2.37 (s, 3H), 2.34-2.26 (m, 4H), 2.13-2.04 (m, 1H), 1.95-1.86 (m, 4H), 1.61-1.58 (m, 2H), 1.55-1.46 (m, 8H), 1.32-1.26 (m, 40H), 0.90-0.86 (m, 12H).

### Example 1.10: Synthesis of CAT10

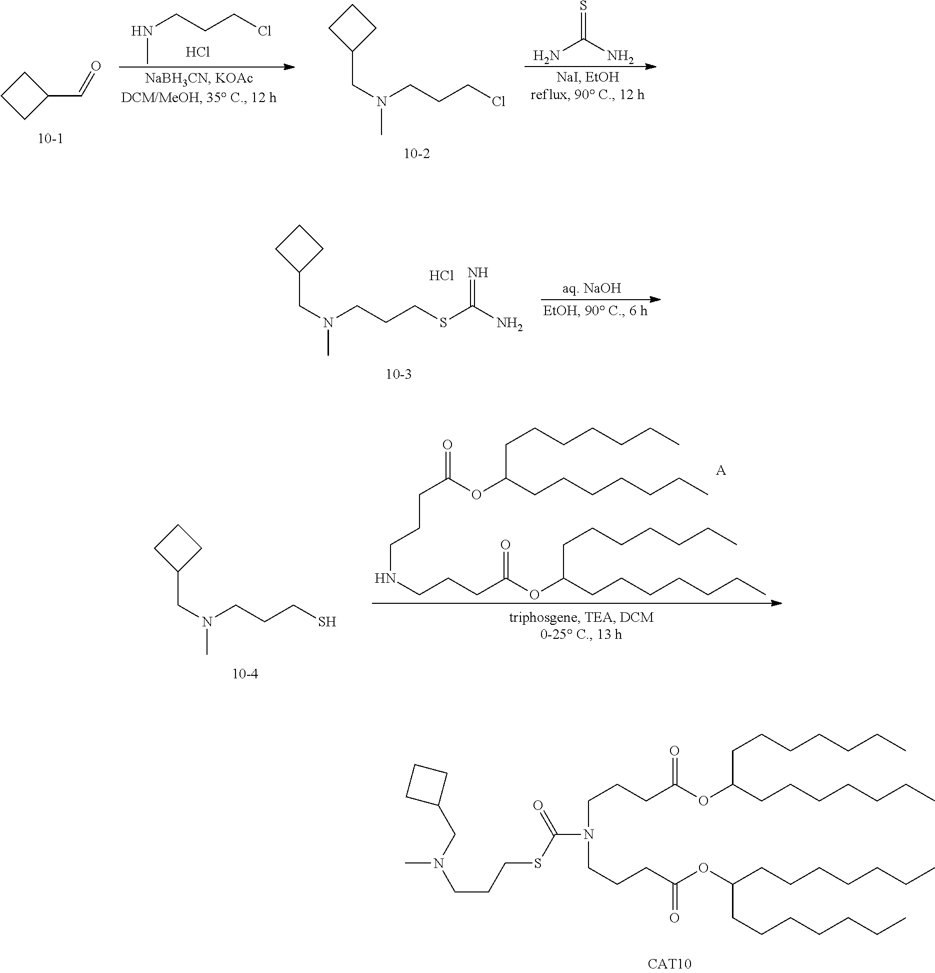

### Step 1: 3-chloro-N-(cyclobutylmethyl)-N-methyl-propan-1-amine (10-2)

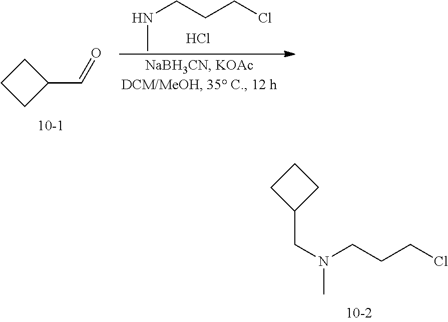

To a solution of cyclobutanecarbaldehyde (29.20 g, 347.12 mmol, 2 eq) and 3-chloro-N-methyl-propan-1-amine;hydrochloride (25 g, 173.56 mmol, 1 eq) in dichlormethane (100 mL) and MeOH (100 mL) were added NaBH₃CN (16.36 g, 260.34 mmol, 1.5 eq) and KOAc (51.10 g, 520.68 mmol, 3 eq). The mixture was stirred at 35° C. for 12 hr. The reaction mixture was quenched by the addition of water (100 mL), and then extracted with ethyl acetate (200 mL×3). The combined organic layers were washed with brine (100 mL), dried over sodium sulfate, filtered and concentrated under reduced pressure. The residue was purified by silica gel chromatography (Petroleum ether/Ethyl acetate=30/1 to 1/1 and Dichloromethane/Methanol=30/1 to 5/1) to give compound 10-2 (27 g, 153.67 mmol, 88.54% yield) as a yellow oil.

¹H NMR (400 MHz, CDCl₃) (5 3.67 (t, J=6.0 Hz, 2H), 3.26-3.20 (m, 2H), 3.12 (d, J=7.2 Hz, 2H), 2.78-2.70 (m, 3H), 2.28-2.18 (m, 4H), 2.10-. 2.05 (m, 1H), 1.90-1.80 (m, 4H).

### Step 2: 2-[3-[cyclobutylmethyl(methyl)amino]propyl]isothiourea (10-3)

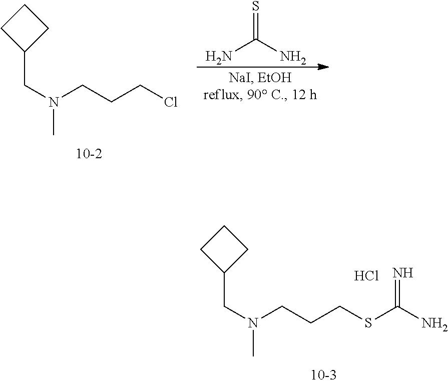

To a solution of 3-chloro-N-(cyclobutylmethyl)-N-methyl-propan-1-amine (10 g, 56.92 mmol, 1 eq) and thiourea (4.77 g, 62.61 mmol, 1.1 eq) in EtOH (100 mL) was added NaI (4.27 g, 28.46 mmol, 0.5 eq). The mixture was stirred at 90° C. for 12 hr under N₂. The reaction mixture was filtered and concentrated under reduced pressure to give compound 10-3 (12 g, 47.65 mmol, 83.73% yield, hydrochloride) as a brown oil.

### Step 3: 3-[cyclobutylmethyl(methyl)amino]propane-1-thiol (10-4)

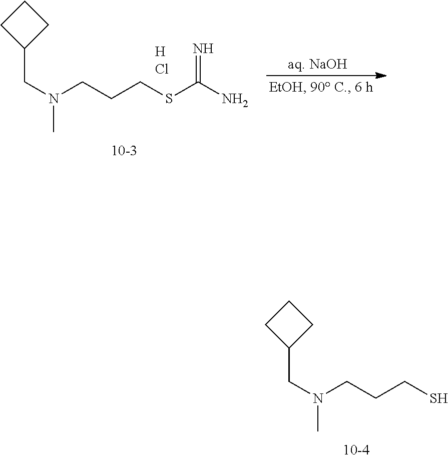

To a solution of 2-[3-[cyclobutylmethyl(methyl)amino]propyl]isothiourea (6 g, 27.86 mmol, 1 eq) in EtOH (30 mL) and water (5 mL) was added NaOH (6.69 g, 167.16 mmol, 6 eq). The mixture was stirred at 90° C. for 6 hr. The reaction mixture was filtered and concentrated under reduced pressure to give compound 10-4 (2.8 g, 16.16 mmol, 57.99% yield) as a yellow oil.

### Step 4: 1-heptyloctyl 4-[3-[cyclobutylmethyl(methyl)amino]propylsulfanylcarbonyl-[4-(]-heptyloctoxy)-4-oxo-butyl]amino]butanoate (CAT10)

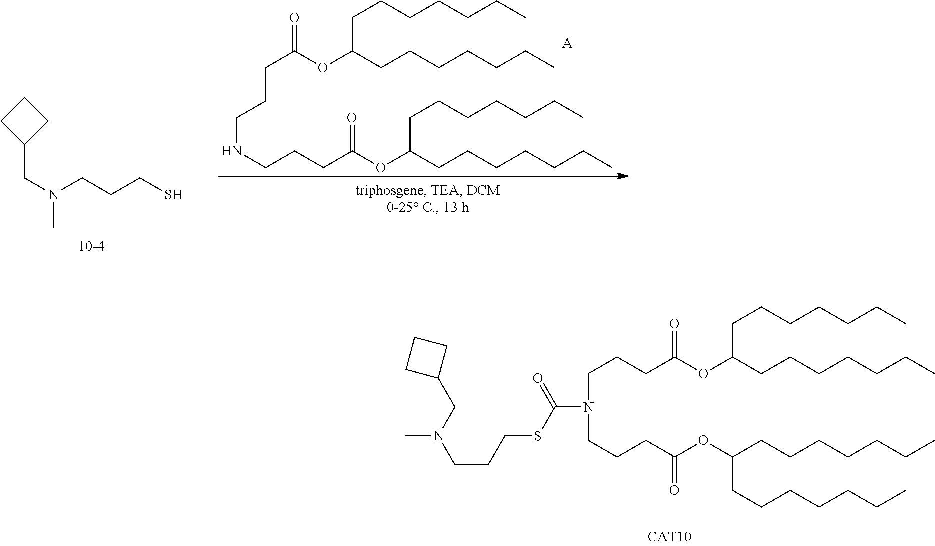

To a solution of 1-heptyloctyl 4-[[4-(1-heptyloctoxy)-4-oxo-butyl]amino]butanoate (1.8 g, 2.95 mmol, 1 eq) dissolved in dry dichlormethane (20 mL) were added TEA (895.77 mg, 8.85 mmol, 1.23 mL, 3 eq) and triphosgene (437.82 mg, 1.48 mmol, 0.5 eq) at 0° C. under nitrogen atmosphere. The resulting solution was stirred at 25° C. under nitrogen atmosphere for 1 hr. The reaction was concentrated under reduced pressure and kept under nitrogen atmosphere. NaOH (826.17 mg, 20.66 mmol, 7 eq) was dissolved in dry THF (60 mL) at 0° C. under nitrogen atmosphere, then 3-[cyclobutylmethyl(methyl)amino]propane-1-thiol (2.56 g, 14.75 mmol, 5 eq) was added under nitrogen atmosphere. To this resulting solution, carbamoyl chloride dissolved in THF (10 mL) was added slowly under nitrogen atmosphere at 0° C. The mixture was stirred at 25° C. for 12 hr. until the reaction was completed. The reaction mixture was quenched by the addition of water (100 mL), and then extracted with ethyl acetate (200 mL×3). The combined organic layers were washed with brine (100 mL), dried over sodium sulfate, filtered and concentrated under reduced pressure, and was purified by silica gel chromatography (Petroleum ether/Ethyl acetate=10/1 to 1/1 and Dichloromethane/Methanol=30/1 to 5/1) and MPLC (Welch Ultimate XB-SiOH 250*50*10 um; mobile phase: [Hexane-EtOH]; B %: 0%-30%, 13 min) to give compound CAT10 (238 mg, 292.02 umol, 11.82% yield, 99.3% purity) as a yellow oil.

### LCMS: [M+H]⁺: 810.0;

¹H NMR (400 MHz, CDCl₃) δ=4.85-4.76 (m, 2H), 3.30-3.25 (m, 4H), 2.84 (t, J=7.2 Hz, 2H), 2.52-2.32 (m, 4H), 2.28-2.12 (m, 7H), 2.05-1.98 (m, 2H), 1.88-1.60 (m, 8H), 1.60-1.52 (m, 3H), 1.48-1.32 (m, 8H), 1.25-1.10 (m, 40H), 0.85-0.78 (m, 12H).

### Example 1.11: Synthesis of CAT11

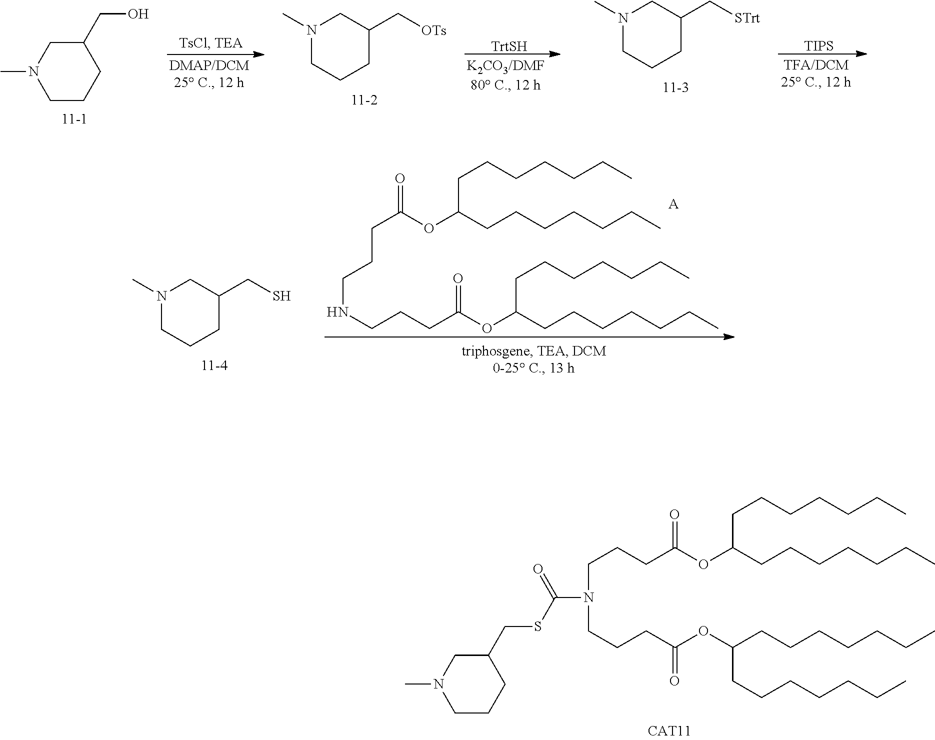

### Step 1: (1-methyl-3-piperidyl)methyl 4-methylbenzenesulfonate (11-2)

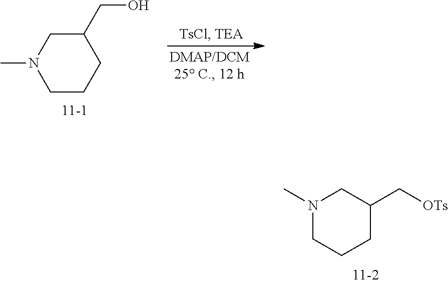

To a solution of (1-methyl-3-piperidyl)methanol (10 g, 77.40 mmol, 1 eq) in dichlormethane (100 mL) were added TosCl (14.76 g, 77.40 mmol, 1 eq) DMAP (945.58 mg, 7.74 mmol, 0.1 eq) and TEA (15.66 g, 154.80 mmol, 21.55 mL, 2 eq). The mixture was stirred at 25° C. for 12 hr. The reaction mixture was quenched by the addition of water (100 mL), and then extracted with ethyl acetate (200 mL×3). The combined organic layers were washed with brine (100 mL), dried over sodium sulfate, filtered and concentrated under reduced pressure, and was purified by silica gel chromatography (Petroleum ether/Ethyl acetate=10/1 to 1/1 and Dichloromethane/Methanol=30/1 to 10/1) to give compound 11-2 as a yellow oil.

¹H NMR (400 MHz, CDCl₃) δ=7.77 (d, J=8.0 Hz, 2H), 7.35 (d, J=8.0 Hz, 2H), 3.96-3.90 (m, 2H), 2.75-2.65 (m, 2H), 2.44 (s, 3H), 2.21 (s, 3H), 1.98-1.90 (m, 2H), 1.70-1.48 (m, 4H), 1.03-0.90 (m, 1H).

### Step 2: 1-methyl-3-(tritylsulfanylmethyl)piperidine (11-3)

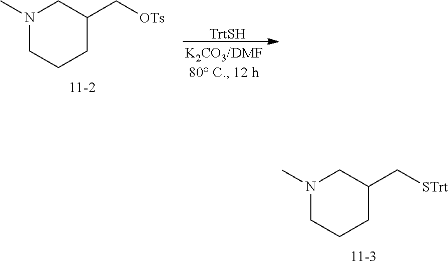

To a solution of (1-methyl-3-piperidyl)methyl 4-methylbenzenesulfonate (7.5 g, 26.47 mmol, 1 eq) and triphenylmethanethiol (8.78 g, 31.76 mmol, 1.2 eq) in DMF (80 mL) was added K₂C₀₃ (10.97 g, 79.40 mmol, 3 eq). The mixture was stirred at 80° C. for 12 hr. The reaction mixture was quenched by the addition of water (200 mL), and then extracted with ethyl acetate (200 mL×3). The combined organic layers were washed with brine (100 mL), dried over sodium sulfate, filtered and concentrated under reduced pressure, and was purified by silica gel chromatography (Petroleum ether/Ethyl acetate=10/1 to 1/1 and Dichloromethane/Methanol=30/1 to 10/1) to give compound 11-3 (7.5 g, 19.35 mmol, 73.12% yield) as a yellow oil.

¹H NMR (400 MHz, CDCl₃) δ=7.49-7.45 (m, 5H), 7.38-7.25 (m, 10H), 2.83-2.80 (m, 2H), 2.32-2.28 (m, 3H), 2.18-2.11 (m, 2H), 1.93-1.90 (m, 1H), 1.77-1.62 (m, 5H), 0.95-0.88 (m, 1H).

### Step 3: (1-methyl-3-piperidyl)methanethiol (11-4)

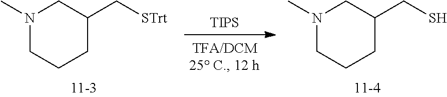

To a solution of 1-methyl-3-(tritylsulfanylmethyl)piperidine (6.5 g, 16.77 mmol, 1 eq) in dichlormethane (50 mL) were added TFA (37.06 g, 325.00 mmol, 30 mL, 19.38 eq) and triisopropylsilane (5.31 g, 33.54 mmol, 6.89 mL, 2 eq) at 0° C. The mixture was stirred at 25° C. for 12 hr. The reaction mixture was concentrated under reduced pressure to remove TFA, it was diluted with MeOH (100 mL) and extracted with Petroleum ether (50 mL×5). The MeOH layers was concentrated under reduced pressure to give compound 11-4 (2.2 g, 15.14 mmol, 90.30% yield) as a yellow oil.

¹H NMR (400 MHz, CDCl₃) δ=3.58-3.52 (m, 2H), 2.79 (s, 3H), 2.60-2.51 (m, 3H), 2.26-2.24 (m, 1H), 2.10-1.75 (m, 4H), 1.40-1.37 (m, 1H), 1.25-1.15 (m, 1H).

### Step 4: 1-heptyloctyl 4-[[4-(1-heptyloctoxy)-4-oxo-butyl]-[(I-methyl-3-piperidyl)methylsulfanylcarbonyl]amino]butanoate (CAT11)

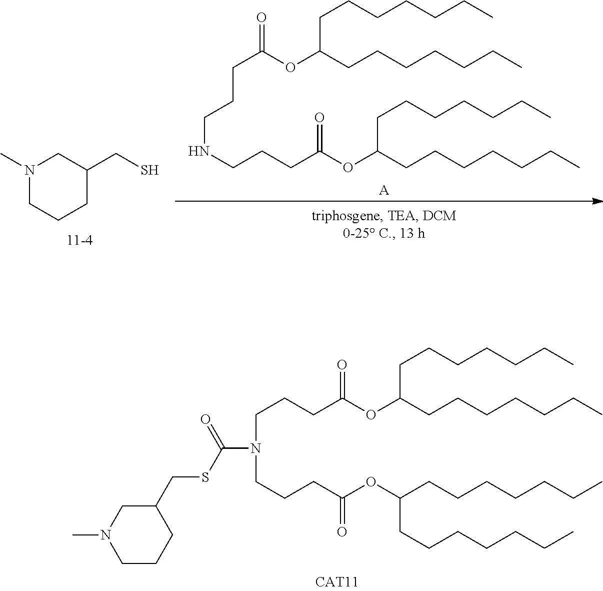

To a solution of 1-heptyloctyl 4-[[4-(1-heptyloctoxy)-4-oxo-butyl]amino]butanoate (1.8 g, 2.95 mmol, 1 eq) dissolved in dry dichlormethane (20 mL) were added TEA (895.77 mg, 8.85 mmol, 1.23 mL, 3 eq) and triphosgene (437.82 mg, 1.48 mmol, 0.5 eq) at 0° C. under nitrogen atmosphere. The resulting solution was stirred at 25° C. under nitrogen atmosphere for 1 hr. The reaction was concentrated under reduced pressure and kept under nitrogen atmosphere. NaOH (826.17 mg, 20.66 mmol, 7 eq) was dissolved in dry THF (30 mL) at 0° C. under nitrogen atmosphere, then (1-methyl-3-piperidyl)methanethiol (2.14 g, 14.75 mmol, 5 eq) was added under nitrogen atmosphere. To this resulting solution, carbamoyl chloride dissolved in THF (10 mL) was added slowly under nitrogen atmosphere at 0° C. The mixture was stirred at 25° C. for 12 hr. The reaction mixture was quenched with saturated aqueous NH₄C₁ (100 mL) and then diluted with ethyl acetate (100 mL). The aqueous phase was extracted with ethyl acetate (100 mL×3). The combined organic phase was washed with brine (100 mL), dried with anhydrous Na₂SO₄, filtered and concentrated in vacuum to give residue. The residue was purified by silica gel chromatography (Petroleum ether/Ethyl acetate=10/1 to 1/1 and Dichloromethane/Methanol=30/1 to 10/1) and purified by column Welch Ultimate XB-SiOH 250*50*10 um; mobile phase: [Hexane-EtOH]; B %: 0%-25%, 20 min to give compound CAT11 (300 mg, 383.61 umol, 16.65% yield, 99.9% purity) as a yellow oil.

### LCMS: [M+H]⁺: 782.1;

¹H NMR (400 MHz, CDCl₃) δ=4.90-4.85 (m, 2H), 3.48-3.40 (m, 4H), 3.10-2.82 (m, 4H), 2.40-2.28 (m, 6H), 2.10-1.70 (m, 8H), 1.60-1.48 (m, 12H), 1.33-1.20 (m, 40H), 0.90-0.86 (m, 12H).

### Example 1.12: Synthesis of CAT12

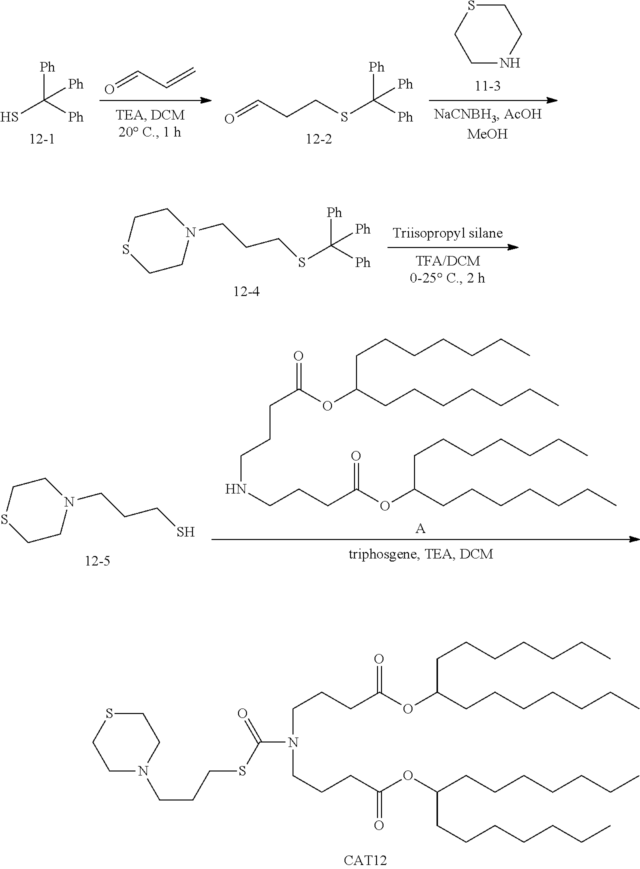

### Step 1: 3-(tritylthio)propanal (12-2)

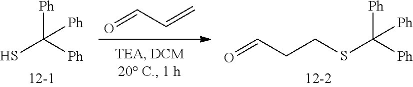

To a mixture of triphenylmethanethiol (10.0 g, 36.2 mmol, 1 eq) in DCM (100 mL) were added TEA (5.13 g, 50.7 mmol, 7.05 mL, 1.4 eq) and prop-2-enal (2.84 g, 50.7 mmol, 3.39 mL, 1.4 eq) successively, the reaction mixture was stirred at 20° C. for 1 hour. The reaction mixture was concentrated under reduced pressure to yield compound 12-2 (12.4 g, crude) as an off-white solid. The reaction residue was used directly for the next step.

### Step 2: 4-(3-(tritylthio)propyl)thiomorpholine (12-4)

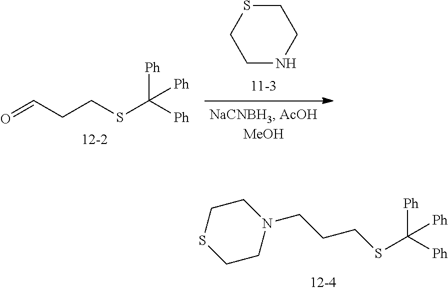 [SMILES: C1CSCCN1 ; O=CCCSC(c1ccccc1)(c1ccccc1)c1ccccc1 ; c1ccc(C(SCCCN2CCSCC2)(c2ccccc2)c2ccccc2)cc1]

To a mixture of 3-tritylsulfanylpropanal (7.40 g, 22.3 mmol, 1 eq) and thiomorpholine (2.53 g, 24.5 mmol, 2.32 mL, 1.1 eq) in MeOH (40 mL) and DCE (40 mL) were added AcOH (134 mg, 2.23 mmol, 0.127 mL, 0.1 eq) and NaBH₃CN (2.80 g, 44.5 mmol, 2 eq) successively, the reaction mixture was stirred at 20° C. for 2 hours. The reaction mixture was quenched by the addition of saturated NH₄Cl solution (50 mL) and extracted by dichloromethane (40 mL×3), then the combined organic phase was dried by anhydrous sodium sulfate, filtered and concentrated under reduced pressure. The crude product was triturated with Petroleum ether/Ethyl acetate=10/1 at 20° C. for 10 min to give compound 12-4 (9.00 g, 21.5 mmol, 96% yield) as a white solid.

### LCMS: [M+H]⁺: 420.0;

¹H NMR (400 MHz, CDCl₃) δ: 7.44-7.28 (m, 12H), 7.24-7.20 (m, 3H), 2.68-2.53 (m, 8H), 2.37-2.25 (m, 2H), 2.19 (t, J=7.2 Hz, 2H), 1.60-1.51 (m, 2H).

### Step 3: 3-thiomorpholinopropane-1-thiol (12-5)

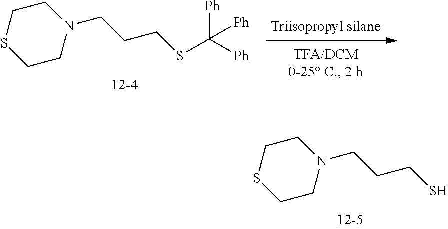 [SMILES: c1ccc(C(SCCCN2CCSCC2)(c2ccccc2)c2ccccc2)cc1 ; SCCCN1CCSCC1]

To a solution of 4-(3-tritylsulfanylpropyl)thiomorpholine (8.00 g, 19.1 mmol, 1 eq) in DCM (10 mL) were added TFA (30.8 g, 270 mmol, 20.0 mL, 14.2 eq) and triisopropylsilane (6.04 g, 38.1 mmol, 7.83 mL, 2 eq) at 0° C. The mixture was stirred at 25° C. for 2 hours. The reaction mixture was concentrated under reduced pressure to obtained a residue, then the reaction residue was added to MeOH (20 mL) and washed with petroleum ether (3×10 mL), dried by anhydrous Na₂SO₄, filtered and concentrated under vacuum to yield compound 12-5 as a yellow oil. The reaction residue was used directly for the next step.

### Step 4: di(pentadecan-8-yl) 4,4′-((((3-thiomorpholinopropyl)thio)carbonyl)azanediyl)dibutanoate (CAT12)

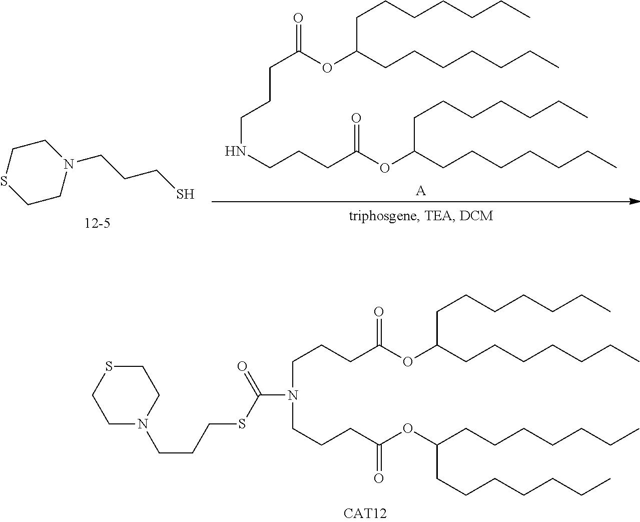

To a solution of 1-heptyloctyl 4-[[4-(1-heptyloctoxy)-4-oxo-butyl]amino]butanoate (2.00 g, 3.28 mmol, 1 eq) dissolved in dry DCM (30 mL) were added TEA (995 mg, 9.84 mmol, 1.37 mL, 3 eq) and bis(trichloromethyl) carbonate (486 mg, 1.64 mmol, 0.5 eq) at 0° C. under N₂. The resulting solution was stirred at 20° C. for 1 hour. The resulting reaction was concentrated under reduced pressure and kept under N₂. To a solution of 3-thiomorpholinopropane-1-thiol (2.33 g, 13.1 mmol, 4 eq) in dry THF (30 mL) was added NaOH (918 mg, 23.0 mmol, 7 eq) at 0° C. under N₂. To this resulting solution, carbamoyl chloride was added via syringe slowly under N₂ at 0° C. The resulting solution was stirred at 20° C. for 3 hours. The reaction mixture was quenched with saturated aqueous NH₄C₁ (60 mL) and then diluted with ethyl acetate (50 mL). The aqueous phase was extracted with ethyl acetate (50 mL×3). The combined organic phase was washed with brine (100 mL), dried with anhydrous Na₂SO₄, filtered and concentrated under vacuum to give a residue. The residue was purified by column chromatography (SiO₂, Petroleum ether/Ethyl acetate=50/1 to 3/1). Compound CAT12 (1.50 g, 1.84 mmol, 56% yield) was obtained as a yellow oil.

### LCMS: [M+H]⁺: 813.6;

¹H NMR (400 MHz, CDCl₃) δ: 4.89-4.86 (m, 2H), 3.40-3.36 (m, 4H), 2.91 (t, J=7.2 Hz, 2H), 2.72-2.68 (m, 6H), 2.48-2.44 (m, 2H), 2.31 (m, 4H), 1.98-1.76 (m, 6H), 1.64-1.45 (m, 1OH), 1.32-1.22 (m, 40H), 0.92-0.83 (m, 12H).

### Example 1.13: Synthesis of CAT13

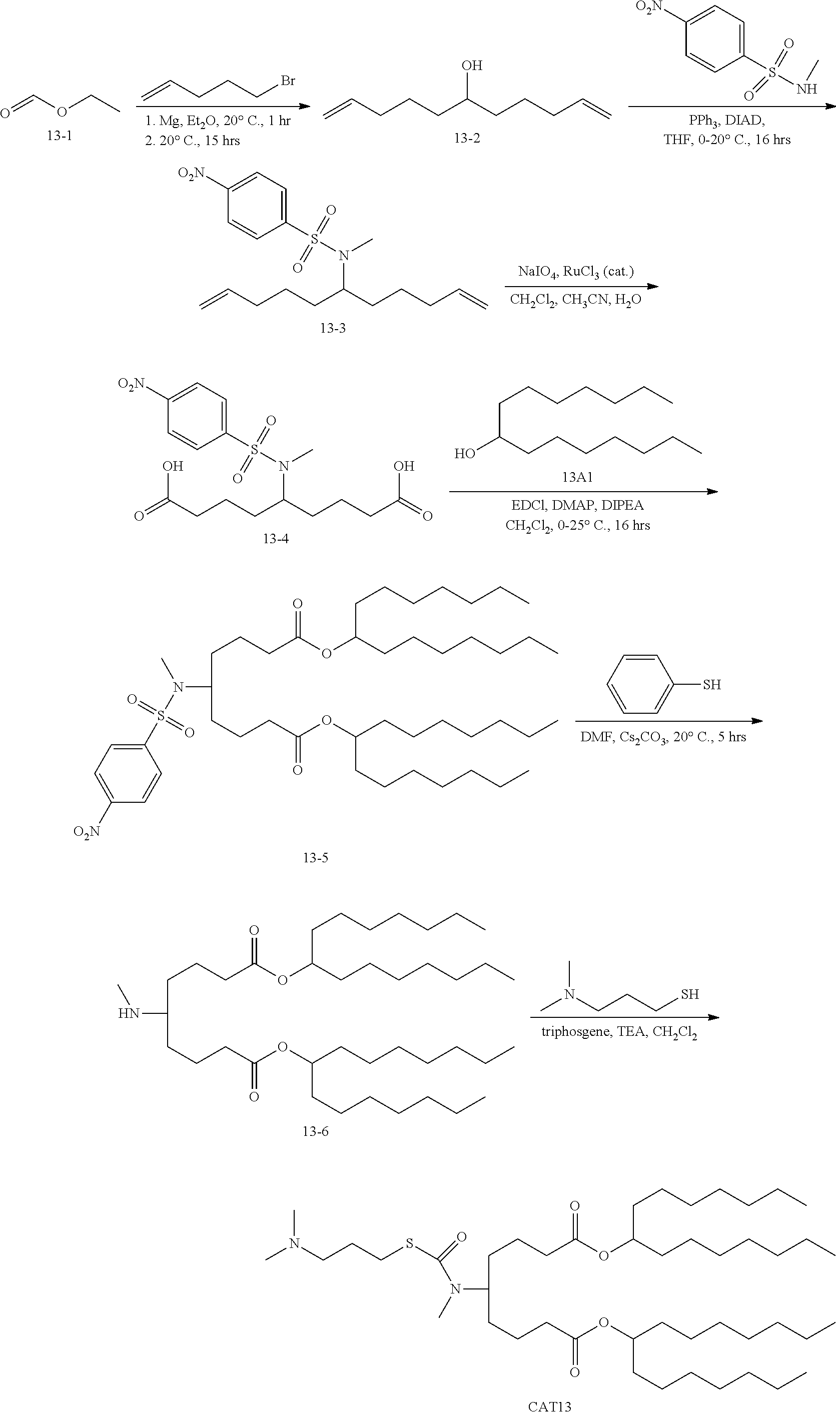

### Step 1: undeca-1,10-dien-6-ol (13-2)

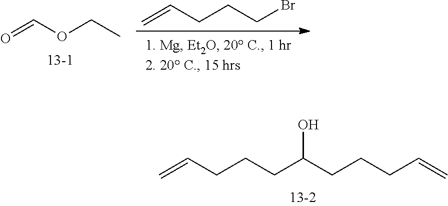

A suspension of Mg (24.61 g, 1.01 mol, 2.5 eq) and 12 (2.06 g, 8.10 mmol, 1.63 mL, 0.02 eq) in dry THF (1500 mL) (2 mL/mmol of bromide) was prepared under nitrogen atmosphere. To this mixture, 5-bromopent-1-ene (150.88 g, 1.01 mol, 2.5 eq) was slowly added at 20° C. While the addition, an increase in the temperature of the reaction mixture confirmed the initiation of the Grignard formation. Once the addition of the bromide was completed, the mixture was stirred at 20° C. for 1 hr, after which it was cooled down to 0° C. for the slow addition of ethyl formate (30 g, 404.98 mmol, 32.6 mL, 1 eq). After the addition, the cold bath was removed and the mixture was stirred at 20° C. for 15 hr. After completion, the reaction was cooled down to 0° C. for quenching by the addition of saturated solution NH₄Cl (1000 mL) and stirred for 30 min. The aqueous phase was extracted with EtOAc (800 mL*3). The combined organic phase was washed with brine (400 mL*2), dried with anhydrous Na₂SO₄, filtered and concentrated in vacuum to give residue. The residue was purified by MPLC (EtOAc: PE: 0˜5%) to give compound 13-2 (53.2 g, 316.15 mmol, 81.9% yield) as a yellow liquid.

¹H NMR (400 MHz, CDCl₃) δ=5.85-5.77 (m, 2H), 5.04-4.95 (m, 4H), 3.62-3.60 (m, 1H), 2.08-2.07 (m, 4H), 1.50-1.42 (m, 8H).

Step 2: N-methyl-4-nitro-N-(undeca-1,10-dien-6-yl)benzenesulfonamide (13-3)

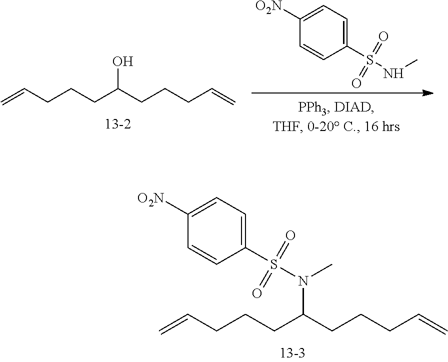 [SMILES: C=CCCCC(O)CCCC=C ; CNS(=O)(=O)c1ccc([N+](=O)[O-])cc1 ; C=CCCCC(CCCC=C)N(C)S(=O)(=O)c1ccc([N+](=O)[O-])cc1]

A solution of undeca-1,10-dien-6-ol (20 g, 118.85 mmol, 1 eq), N-methyl-4-nitro-benzenesulfonamide (28.27 g, 130.74 mmol, 1.1 eq) and PPh3 (37.41 g, 142.62 mmol, 1.2 eq) was stirred in dry THF (200 mL) a(0° C. under N₂ To this mixture was added dropwise DIAD (36.05 g. 178.28 mmol. 34.7 mL, 1.5 eq) in THF (30 mL) over a period of 0.5 hr After addition, the resulting mixture was stirred at 20° C. 15.5 hr. After completion, the reaction mixture was quenched by H₂O (150 mL) and then diluted with EtOAc (100 mL). The aqueous phase was extracted with EtOAc (150 mL*3). The combined organic phase was washed with brine (50 mL), dried with anhydrous Na₂SO₄, filtered and concentrated in vacuum to give a residue. The residue was purified by flash silica gel chromatography (330 g SepaFlash®Silica Flash Column, EtOAc: PE: 0˜10%) to give compound 13-3 (33.2 g, 90.59 mmol, 76.2% yield) as a yellow oil.

¹H NMR (400 MHz, CDCl₃) δ=8.36-8.33 (m, 2H), 8.01-7.98 (m, 2H), 5.74-5.65 (m, 2H), 4.97-4.92 (m, 4H), 3.92-3.87 (m, 1H), 2.70 (s, 3H), 2.01-1.97 (m, 4H), 1.30-1.28 (m, 2H), 1.27-1.23 (m, 6H).

### Step 3: 5-(N-methyl-4-nitrophenylsulfonamido)nonanedioic acid (13-4)

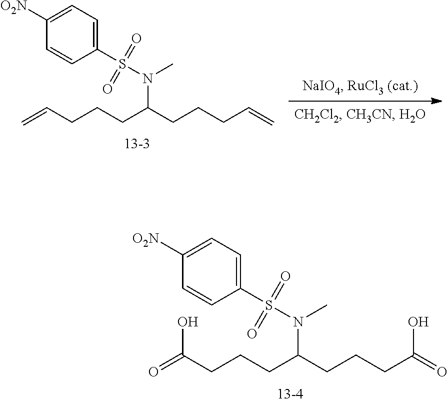 [SMILES: C=CCCCC(CCCC=C)N(C)S(=O)(=O)c1ccc([N+](=O)[O-])cc1 ; CN(C(CCCC(=O)O)CCCC(=O)O)S(=O)(=O)c1ccc([N+](=O)[O-])cc1]

To a solution of N-methyl-4-nitro-N-(1-pent-4-enylhex-5-enyl)benzenesulfonamide (12.5 g, 34.11 mmol, 1 eq) in MeCN (150 mL) and CH₂Cl₂ (150 mL) was added RuCl3 (1.42 g, 6.82 mmol, 0.2 eq) at 20° C. After addition, the mixture was stirred at this temperature for 0.5 hr, and then NaIO₄ (36.48 g, 170.54 mmol, 5 eq) in H₂O (200 mL) was added dropwise at 0° C. The resulting mixture was stirred at 20° C. for 2.5 hr. After completion, the reaction mixture was neutralized to pH=2-3 with aq.HCl (4 M). The aqueous phase was extracted with EtOAc (600 mL*3). The combined organic phase successively was washed with saturated aqueous Na₂S203 (350 mL*3) and saturated brine (350 mL*2), dried over Na₂SO₄, filtered and concentrated in vacuum to give residue. The residue was purified by flash silica gel chromatography (120 g SepaFlash® Silica Flash Column, EtOAc: PE: 0˜50%, 2% AcOH in EtOAc) and prep-HPLC (column: YMC Triart C18 250*50 mm*7 um; mobile phase: [water (0.05% HCl)-ACN]; B %: 25%-55%, 18 min) to give compound 13-4 (5.2 g, 12.92 mmol, 20.8% yield) as a light yellow solid.

¹H NMR (400 MHz, DMSO-d₆) δ=11.98 (s, 2H), 8.41-8.38 (m, 2H), 8.07-8.05 (m, 2H), 3.76-3.74 (m, 1H), 2.67 (s, 3H), 2.16-2.11 (m, 4H), 1.35-1.22 (m, 8H).

### Step 4: di(pentadecan-8-yl) 5-(N-methyl-4-nitrophenylsulfonamido)nonanedioate (13-5)

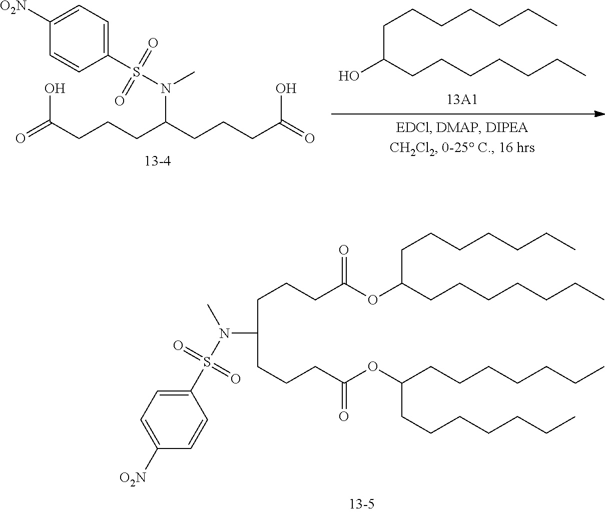 [SMILES: CN(C(CCCC(=O)O)CCCC(=O)O)S(=O)(=O)c1ccc([N+](=O)[O-])cc1 ; CCCCCCCC(O)CCCCCCC ; CCCCCCCC(CCCCCCC)OC(=O)CCCC(CCCC(=O)OC(CCCCCCC)CCCCCCC)N(C)S(=O)(=O)c1ccc([N+](=O)[O-])cc1]

A solution of 5-[methyl-(4-nitrophenyl)sulfonyl-amino]nonanedioic acid (5 g, 12.42 mmol, 1 eq) dissolved in CH₂Cl₂ (80 mL) were added EDCI (7.15 g, 37.27 mmol, 3 eq), TEA (3.77 g, 37.27 mmol, 5.2 mL, 3 eq) and DMAP (1.52 g, 12.42 mmol, 1 eq) at 0° C. under N₂. After addition, the mixture was stirred at 25° C. for 1 hr, and then pentadecan-8-ol (5.96 g, 26.09 mmol, 2.1 eq) in CH₂Cl₂ (50 mL) was added dropwise. The resulting mixture was stirred at 25° C. for 15 hr. After completion, The reaction mixture was quenched by H₂O (100 mL) and then diluted with EtOAc (50 mL). The aqueous phase was extracted with EtOAc (80 mL*3). The combined organic phase was washed with brine (60 mL), dried with anhydrous Na₂SO₄, filtered and concentrated in vacuum to give residue The residue was purified by flash silica gel chromatography (40 g SepaFlash® Silica Flash Column, EtOAc: PE: 0-10% to give compound 13-5 (4.9 g, 5.89 mmol, 47.4% yield, 99% purity) as a yellow oil.

### LCMS [M+Na] *: 845.5;

¹H NMR (400 MHz, CDCl₃) δ=8.36 (d, J=8.8 Hz, 2H), 8.01 (d, J=8.8 Hz, 2H), 4.86-4.80 (m, 2H), 3.96-3.91 (m, 1H), 2.72 (s, 3H), 2.31-2.19 (m, 4H), 1.50-1.45 (m, 14H), 1.28-1.26 (m, 42H), 0.90-0.87 (m, 12H).

### Step 5: di(pentadecan-8-yl) 5-(methylamino)nonanedioate (13-6)

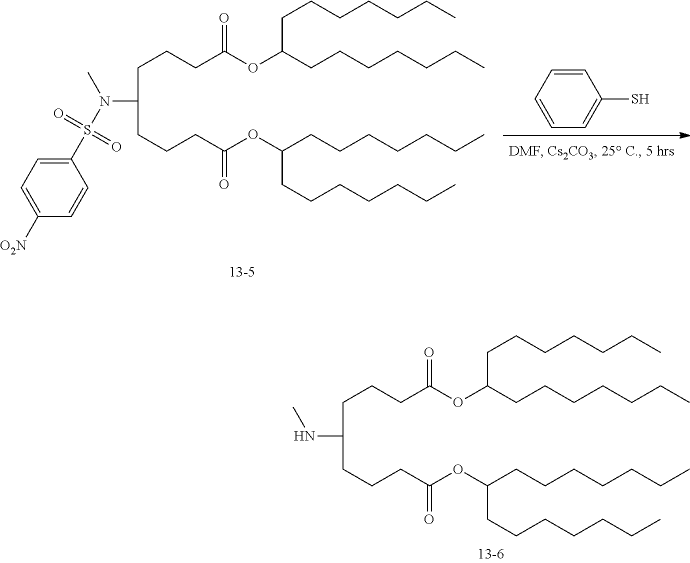

To a solution of bis(1-heptyloctyl) 5-[methyl-(4-nitrophenyl)sulfonyl-amino]nonanedioate (4.9 g, 5.95 mmol, 1 eq) in DMF (40 mL) were added Cs₂CO₃ (3.88 g, 11.90 mmol, 2 eq) and benzenethiol (1.94 g, 17.61 mmol, 1.8 mL, 2.96 eq). The mixture was stirred at 25° C. for 5 hr. After completion, the reaction mixture was quenched by the addition of water (80 mL), and then extracted with EtOAc (100 mL*3). The combined organic layers were washed with brine (60 mL*3), dried over sodium sulfate, filtered and concentrated under reduced pressure. The residue was purified by flash silica gel chromatography (20 g SepaFlash® Silica Flash Column, EtOAc: PE: 0˜60%) to give compound 13-6 (2.6 g, 4.07 mmol, 68.5% yield) as a yellow oil.

¹H NMR (400 MHz, CDCl₃) δ=4.90-4.84 (m, 2H), 2.47-2.45 (m, 1H), 2.40 (s, 3H), 2.32-2.29 (m, 4H), 1.67-1.63 (m, 4H), 1.51-1.45 (m, 12H), 1.43-1.27 (m, 40H), 0.90-0.87 (m, 12H).

### Step 6: di(pentadecan-8-yl) 5-((((3-(dimethylamino)propyl)thio)carbonyl)(methyl)amino)nonanedioate (CAT13)

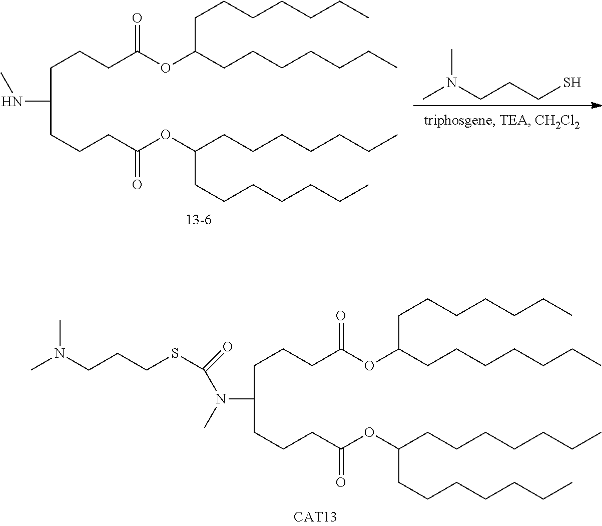

To a solution of bis(1-heptyloctyl) 5-(methylamino)nonanedioate (1.5 g, 2.35 mmol, 1 eq) dissolved in dry CH₂Cl₂ (30 mL) were added TEA (713.7 mg, 7.05 mmol, 0.98 mL, 3 eq) and triphosgene (418.6 mg, 1.41 mmol, 0.6 eq) at 0° C. under N₂. The resulting solution was stirred at 20° C. for 1 hr. The resulting reaction was concentrated under reduced pressure and kept under N₂. To a solution of 3-(dimethylamino)propane-1-thiol (981.0 mg, 8.23 mmol, 3.5 eq) dissolved in dry THF (30 mL) was added NaOH (658.3 mg, 16.46 mmol, 7 eq) at 0° C. under N₂. To this resulting solution, carbamoyl chloride, dissolved in THF (20 mL), was added via syringe slowly under N₂ at 0° C. The resulting solution was stirred at 20° C. for 2 hr. After completion, the reaction mixture was quenched by NH₄Cl (60 mL) at 0° C. and then diluted with EtOAc (50 mL). The aqueous phase was extracted with EtOAc (60 mL*3). The combined organic phase was washed with brine (50 mL), dried with anhydrous Na₂SO₄, filtered and concentrated in vacuum to give residue. The residue was purified by flash silica gel chromatography (20 g SepaFlash® Silica Flash Column, EtOAc: PE: 0˜12%, 5% NH₃·H₂O in Ethyl acetate) to afford compound CAT13 (1.05 g, 1.32 mmol, 56.2% yield, 98.5% purity) as a light yellow oil.

### LCMS [M+H]⁺: 783.6

¹H NMR (400 MHz, CDCl₃) δ=4.86-4.82 (m, 2H), 4.55-3.84 (m, 1H), 2.93-2.92 (m, 2H), 2.80-2.78 (m, 3H), 2.36-2.30 (m, 6H), 2.23 (s, 6H), 1.81-1.77 (m, 3H), 1.50-1.45 (m, 16H), 1.32-1.26 (m, 40H), 0.90-0.87 (m, 12H).

### Example 1.14: Synthesis of CAT14

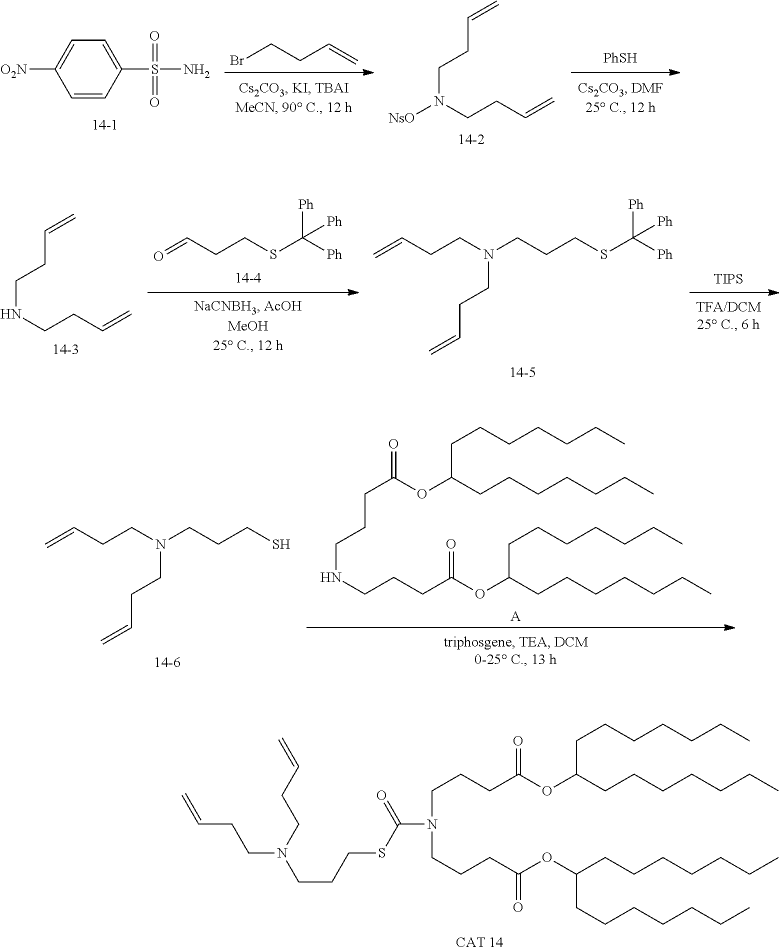

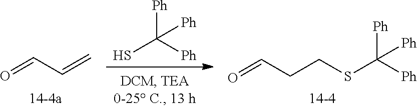

### Step 1: N,N-bis(but-3-enyl)-4-nitro-benzenesulfonamide (14-2)

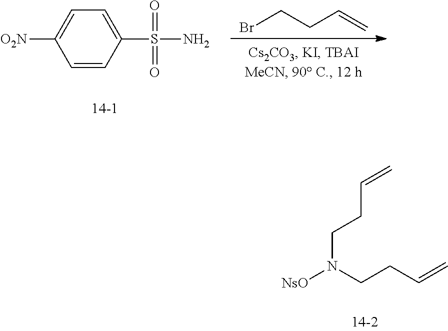

To a solution of 4-nitrobenzenesulfonamide (25 g, 123.65 mmol, 1 eq) and 4-bromobut-1-ene (83.46 g, 618.24 mmol, 62.75 mL, 5 eq) in ACN (50 mL) were added Cs₂CO₃ (80.57 g, 247.30 mmol, 2 eq), TBAI (456.71 mg, 1.24 mmol, 0.01 eq) and KI (10.26 g, 61.82 mmol, 0.5 eq). The mixture was stirred at 90° C. for 12 hr. The reaction mixture was quenched by the addition of water (300 mL), and then extracted with EtOAc (500 mL×3). The combined organic layers were washed with brine (200 mL), dried over sodium sulfate, filtered and concentrated under reduced pressure, and was purified by silica gel chromatography (Petroleum ether/Ethyl acetate=10/1 to 1/1) to give compound 14-2 (37 g, 119.21 mmol, 96.4% yield) as a yellow oil.

¹H NMR (400 MHz, CDCl₃) δ=8.37-8.34 (m, 2H), 8.03-7.99 (m, 2H), 5.73-5.64 (m, 2H), 5.10-5.04 (m, 4H), 3.30-3.25 (m, 4H), 2.35-2.30 (m, 4H)

### Step 2: N-but-3-enylbut-3-en-1-amine: (14-3)

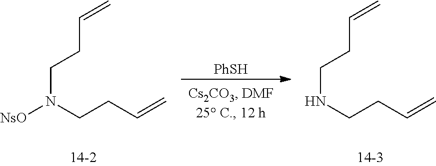

To a solution of N,N-bis(but-3-enyl)-4-nitro-benzenesulfonamide (74 g, 238.43 mmol, 1 eq) and benzenethiol (52.54 g, 476.85 mmol, 48.65 mL, 2 eq) in DMF (200 mL) was added Cs₂CO₃ (155.37 g, 476.85 mmol, 2 eq). The mixture was stirred at 25° C. for 12 hr under N₂. The reaction mixture was quenched by the addition of water (1000 mL), and then extracted with EtOAc (1000 mL×3). The combined organic layers were washed with brine (2000 mL), dried over sodium sulfate, filtered and concentrated under reduced pressure, and was purified by silica gel chromatography (Petroleum ether/Ethyl acetate=10/1 to 1/1) to give compound 14-3 (44 g, crude) as a yellow oil.

¹H NMR (400 MHz, CDCl₃) δ=5.81-5.73 (m, 2H), 5.08-4.99 (m, 4H), 2.66 (t, J=6.8 Hz, 4H), 2.26-2.20 (m, 4H), 1.39-1.36 (m, 1H)

### Step 3: 3-(tritylthio)propanal: (14-4)

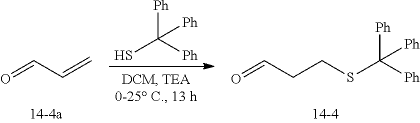

To a solution of triphenylmethanethiol (50 g, 180.90 mmol, 1 eq) in CH₂Cl₂ (300 mL) were added TEA (27.46 g, 271.35 mmol, 37.77 mL, 1.5 eq) and prop-2-enal (15.21 g, 271.35 mmol, 18.0 mL, 1.5 eq). The mixture was stirred at 20° C. for 12 hr. The reaction mixture was concentrated under reduced pressure to give compound 3-tritylsulfanylpropanal (60 g, 180.47 mmol, 99.76% yield) as a yellow solid.

¹H NMR (400 MHz, CDCl₃) δ=9.46 (s, 1H), 7.40-7.20 (m, 15H), 2.46-2.41 (m, 2H), 2.35-2.29 (m, 2H)

### Step 4: N-but-3-enyl-N-(3-tritylsulfanylpropyl)but-3-en-1-amine: (14-5)

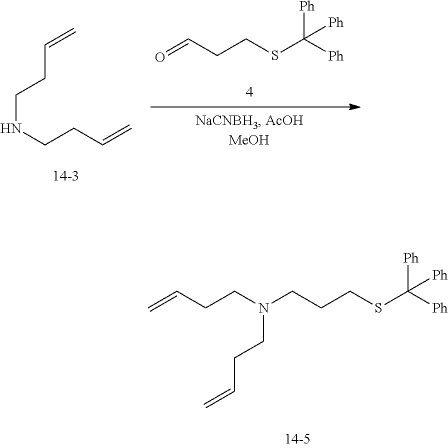

To a solution of N-but-3-enylbut-3-en-1-amine (30 g, 239.60 mmol, 1 eq) and 3-tritylsulfanylpropanal (79.66 g, 239.60 mmol, 1 eq) in CH₂Cl₂ (100 mL) and MeOH (100 mL) were added NaBH₃CN (30.11 g, 479.19 mmol, 2 eq) and AcOH (1.44 g, 23.96 mmol, 1.37 mL, 0.1 eq). The mixture was stirred at 25° C. for 12 hr. The reaction mixture was quenched by the addition of water (300 mL), and then extracted with EtOAC (500 mL×3). The combined organic layers were washed with brine (200 mL), dried over sodium sulfate, filtered and concentrated under reduced pressure, and was purified by silica gel chromatography (Petroleum ether/Ethyl acetate=10/1 to 1/1) to give compound N-but-3-enyl-N-(3-tritylsulfanylpropyl)but-3-en-1-amine (46 g, 104.15 mmol, 43.47% yield) as a yellow oil.

¹H NMR (400 MHz, CDCl₃) δ=7.51-7.29 (m, 15H), 5.86-5.79 (m, 2H), 5.12-5.03 (m, 4H), 2.53-2.45 (m, 6H), 2.28-2.20 (m, 6H), 1.63-1.57 (m, 2H)

### Step 5: N-but-3-enyl-N-(3-tritylsulfanylpropyl)but-3-en-1-amine: (14-6)

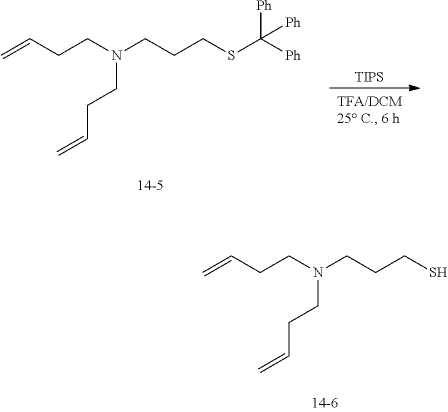 [SMILES: C=CCCN(CCC=C)CCCSC(c1ccccc1)(c1ccccc1)c1ccccc1 ; C=CCCN(CCC=C)CCCS ; "TIPS"]

To a solution of N-but-3-enyl-N-(3-tritylsulfanylpropyl)but-3-en-1-amine (30 g, 67.92 mmol, 1 eq) in CH₂Cl₂ (100 mL) were added TFA (231.00 g, 2.03 mol, 150.00 mL, 29.83 eq) and triisopropylsilane (21.51 g, 135.85 mmol, 27.90 mL, 2 eq). The mixture was stirred at 25° C. for 6 hr. The reaction mixture was concentrated under reduced pressure to remove TFA. The residue was diluted with MeOH (100 mL) and extracted with PE (50 mL×5). The MeOH layers was concentrated under reduced pressure to give crude product 14-6 (9.8 g, 49.16 mmol, 72.37% yield) as a yellow oil.

### Step 6: 1-heptyloctyl 4-[3-[bis(but-3-enyl)amino]propylsulfanylcarbonyl-[4-(1-heptyloctoxy)-4-oxo-butyl]amino]butanoate: (CAT 14)

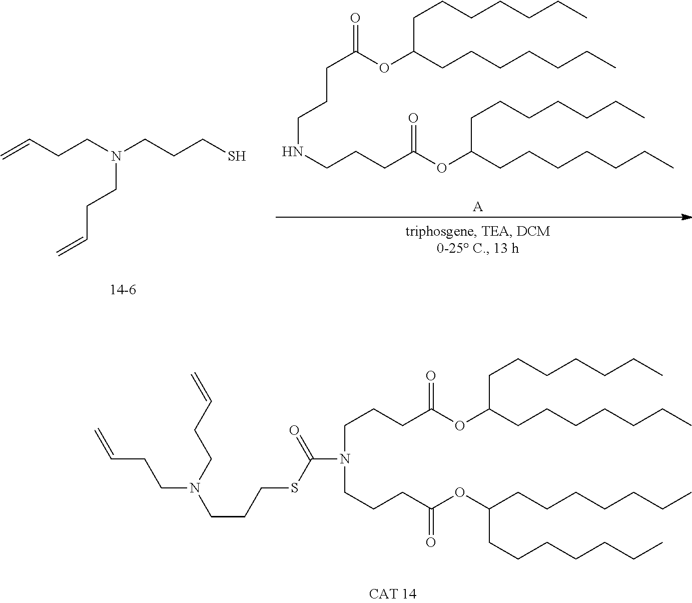

To a solution of 1-heptyloctyl 4-[[4-(1-heptyloctoxy)-4-oxo-butyl]amino]butanoate (6 g, 9.84 mmol, 1 eq) dissolved in dry CH₂Cl₂ (50 mL) were added TEA (2.99 g, 29.51 mmol, 4.11 mL, 3 eq) and triphosgene (1.46 g, 4.92 mmol, 0.5 eq) at 0° C. under nitrogen atmosphere. The resulting solution was stirred at 20° C. under nitrogen atmosphere for 1 hr. The reaction was concentrated under reduced pressure and kept under nitrogen atmosphere. NaOH (2.75 g, 68.85 mmol, 7 eq) was dissolved in dry THF (100 mL) at 0° C. under nitrogen atmosphere, then 3-[bis(but-3-enyl)amino]propane-1-thiol (9.80 g, 49.18 mmol, 5 eq) was added under nitrogen atmosphere. To this resulting solution, carbamoyl chloride dissolved in THF (50 mL) was added slowly under nitrogen atmosphere at 0° C. The mixture was stirred at 25° C. for 12 hr. The reaction mixture was quenched with saturated aqueous NH₄C₁ (200 mL) and then diluted with EtOAC (300 mL). The aqueous phase was extracted with EtOAC (200 mL×3). The combined organic phase was washed with brine (100 mL), dried with anhydrous Na₂SO₄, filtered and concentrated in vacuum to give residue, and was purified by silica gel chromatography (Petroleum ether/Ethyl acetate=10/1 to 1/1) and Phenomenex Luna C8 250*50 mm*10 um; mobile phase: [water (HCl)-MeOH]; B %: 80%-100%, 10 min to give compound CAT14 (240 mg, 284.46 umol, 23.76% yield, 99.01% purity) as a yellow oil.

### LCMS: [M+H]⁺: 836.2;

¹H NMR (400 MHz, CDCl₃) δ=5.83-5.76 (m, 2H), 5.09-5.02 (m, 4H), 4.99-4.86 (m, 2H), 3.40-3.38 (m, 4H), 2.92 (t, J=7.2 Hz, 2H), 2.53-2.31 (m, 6H), 2.30-2.21 (m, 6H), 1.90-1.78 (m, 6H), 1.58-1.51 (m, 1OH), 1.32-1.27 (m, 40H), 0.90-0.87 (m, 12H).

### Example 1.15: Synthesis of CAT15

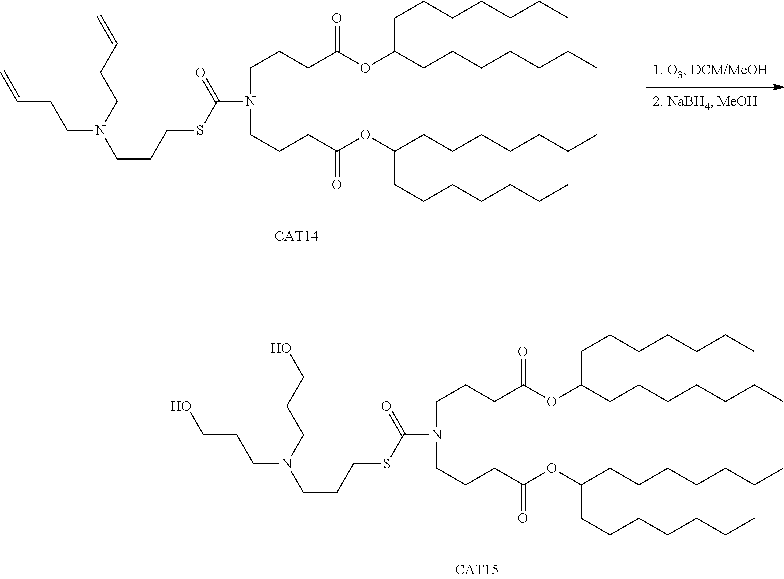 [SMILES: C=CCCN(CCC=C)CCCSC(=O)N(CCCC(=O)OC(CCCCCCC)CCCCCCC)CCCC(=O)OC(CCCCCCC)CCCCCCC ; CCCCCCCC(CCCCCCC)OC(=O)CCCN(CCCC(=O)OC(CCCCCCC)CCCCCCC)C(=O)SCCCN(CCCO)CCCO]

### Step 1: 1-heptyloctyl 4-[3-[bis(3-hydroxypropyl)amino]propylsulfanylcarbonyl-[4-(1-heptyloctoxy)-4-oxo-butyl]amino]butanoate: (CAT15)

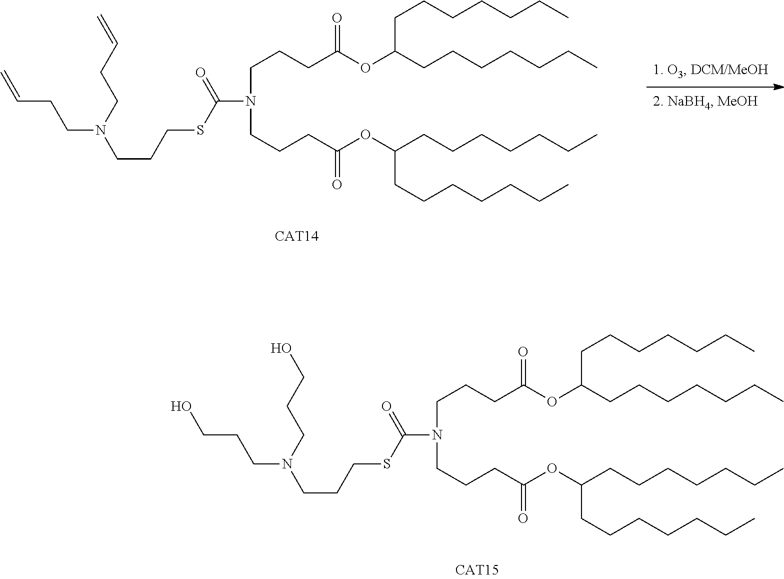 [SMILES: C=CCCN(CCC=C)CCCSC(=O)N(CCCC(=O)OC(CCCCCCC)CCCCCCC)CCCC(=O)OC(CCCCCCC)CCCCCCC ; CCCCCCCC(CCCCCCC)OC(=O)CCCN(CCCC(=O)OC(CCCCCCC)CCCCCCC)C(=O)SCCCN(CCCO)CCCO]

A solution of 1-heptyloctyl 4-[3-[bis(but-3-enyl)amino]propylsulfanylcarbonyl-[4-(1-heptyloctoxy)-4-oxo-butyl]amino]butanoate (2.8 g, 3.35 mmol, 1 eq) in CH₂Cl₂ (50 mL) and MeOH (50 mL) was cooled to −78° C., and a stream of 03 (15 psi) was bubbled into the reaction mixture until a light blue color became evident. Oxygen was then bubbled through the reaction mixture until the blue color disappeared, after 0.5 hr, the NaBH₄ (253.60 mg, 6.70 mmol, 2 eq) was added at 0° C. Then, the mixture was stirred at 25° C. for 2 hr. The reaction mixture was quenched with saturated aqueous NH₄C₁ (100 mL) and then diluted with EtOAc (50 mL). The aqueous phase was extracted with EtOAc (50 mL×3). The combined organic phase was washed with brine (20 mL), dried with anhydrous Na₂SO₄, filtered and concentrated in vacuum to give residue, and was purified by silica gel chromatography (Petroleum ether/Ethyl acetate=10/1 to 1/1) and purified by column: Welch Ultimate XB-SiOH 250*50*10 um; mobile phase: [Hexane-EtOH]; B %: 0%-20%, 20 min to give CAT15 (141 mg, 166.19 umol, 77.86% yield, 99.4% purity) as a yellow oil.

### LCMS: [M+H]⁺: 843.7;

¹H NMR (400 MHz, CDCl₃) δ=4.90-4.65 (m, 2H), 3.82-3.65 (m, 4H), 3.45-3.25 (m, 4H), 3.00-2.90 (m, 6H), 2.38-2.20 (m, 4H), 2.00-1.75 (m, 10H), 1.70-1.55 (m, 10H), 1.30-1.15 (m, 40H), 0.96-0.86 (m, 12H).

### Example 1.16: Synthesis of CAT16

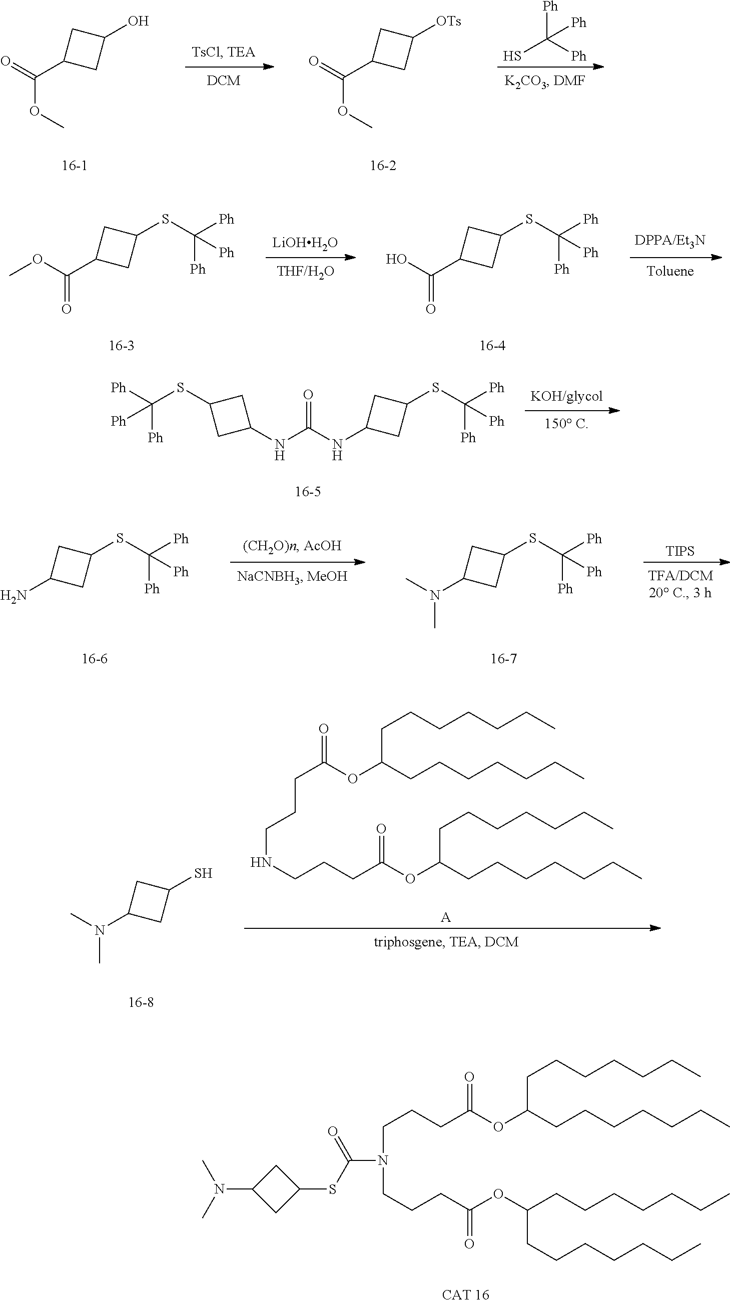

### Step 1: methyl 3-(tosyloxy)cyclobutanecarboxylate (16-2)

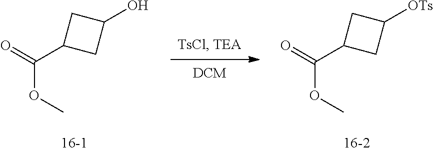

To a solution of methyl 3-hydroxycyclobutanecarboxylate (15.0 g, 115 mmol, 1 eq) in DCM (250 mL) were added TEA (23.3 g, 231 mmol, 32.1 mL, 2 eq), DMAP (704 mg, 5.76 mmol, 0.05 eq) and TosCl (26.4 g, 138 mmol, 1.2 eq) at 0° C. under N₂. The mixture was stirred at 20° C. for 16 hours. The reaction mixture was diluted with DCM (100 mL) and washed with brine (80 mL×3), dried with anhydrous Na₂SO₄, filtered and concentrated under vacuum to give residue. The residue was purified by flash silica gel chromatography (80 g SepaFlash®Silica Flash Column, Ethyl acetate: Petroleum ether: 0˜25%). Compound 16-2 (22.3 g, 78.4 mmol, 68% yield) was obtained as a light yellow oil.

### Step 2: methyl 3-(tritylthio)cyclobutanecarboxylate (16-3)

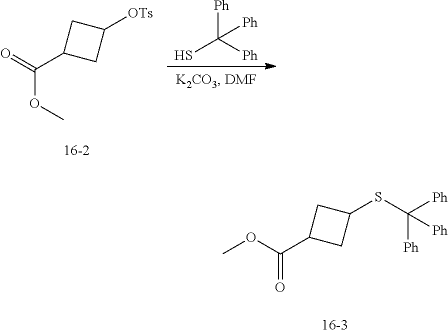 [SMILES: COC(=O)C1CC(OS(=O)(=O)c2ccc(C)cc2)C1 ; SC(c1ccccc1)(c1ccccc1)c1ccccc1 ; COC(=O)C1CC(SC(c2ccccc2)(c2ccccc2)c2ccccc2)C1]

To a solution of methyl 3-(p-tolylsulfonyloxy)cyclobutanecarboxylate (26.0 g, 91.4 mmol, 1 eq) in DMF (300 mL) were added triphenylmethanethiol (37.9 g, 137 mmol, 1.5 eq) and Cs₂CO₃ (59.6 g, 183 mmol, 2 eq). The mixture was stirred at 20° C. for 12 hours. The reaction mixture was quenched by H2O (100 mL) and then diluted with ethyl acetate (200 mL). The aqueous phase was extracted with ethyl acetate (200 mL×2). The combined organic phase was washed with brine (200 mL), dried with anhydrous Na₂SO₄, filtered and concentrated under vacuum to give a crude product. The residue was purified by flash silica gel chromatography (ISCO®; 220 g SepaFlash® Silica Flash Column, Eluent of 0˜15% Ethyl acetate/Petroleum ethergradient @ 40 mL/min). Compound 16-3 (35.0 g, 90.1 mmol, 98% yield) was obtained as a yellow oil.

¹H NMR (400 MHz, CDCl₃) δ: 7.28-7.17 (m, 15H), 3.59 (s, 3H), 3.35-3.30 (m, 1H), 3.00-2.93 (m, 1H), 2.19-2.13 (m, 2H), 2.04-1.99 (m, 2H).

### Step 3: 3-(tritylthio)cyclobutanecarboxylic acid (16-4)

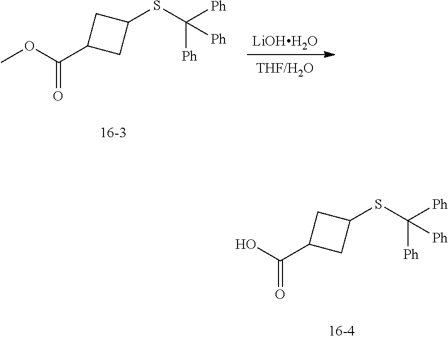

To a mixture of methyl 3-tritylsulfanylcyclobutanecarboxylate (25.0 g, 64.4 mmol, 1 eq) in THF (200 mL) was added LiOH-H₂O (8.10 g, 193.1 mmol, 3 eq), the reaction mixture was stirred at 40° C. for 12 hours. The reaction mixture was adjusted to pH 5 with 4 M HCl, then the reaction mixture was extracted with ethyl acetate (200 mL×3). The combined organic layers were washed with brine (200 mL×2), dried over Na₂SO₄, filtered and concentrated under reduced pressure to give a residue. The residue was purified by flash silica gel chromatography (ISCO®; 20 g SepaFlash® Silica Flash Column, Eluent of 0˜50% Ethyl acetate/Petroleum ethergradient @ 40 mL/min). Compound 16-4 (17.8 g, 47.5 mmol, 74% yield) was obtained as a yellow solid.

¹H NMR (400 MHz, DMSO-d₆) δ:12.07 (s, 1H), 7.38-7.27 (m, 12H), 7.26-7.19 (m, 3H), 3.21-3.13 (m, 1H), 2.87-2.80 (m, 1H), 2.02-1.81 (m, 4H).

### Step 4: 1,3-bis(3-(tritylthio)cyclobutyl)urea (16-5)

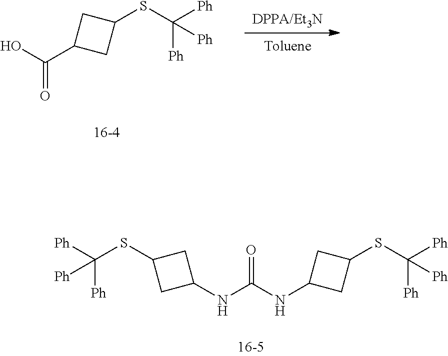 [SMILES: O=C(O)C1CC(SC(c2ccccc2)(c2ccccc2)c2ccccc2)C1 ; "DPPA/Et3N" ; O=C(NC1CC(SC(c2ccccc2)(c2ccccc2)c2ccccc2)C1)NC1CC(SC(c2ccccc2)(c2ccccc2)c2ccccc2)C1]

To a mixture of 3-tritylsulfanylcyclobutanecarboxylic acid (10.0 g, 26.7 mmol, 1 eq) and TEA (4.05 g, 40.1 mmol, 5.57 mL, 1.5 eq) in toluene (100 mL) was added DPPA (8.82 g, 32.0 mmol, 6.94 mL, 1.2 eq) at 20° C., then the reaction mixture was heated to 100° C. and stirred for 4 hours. The reaction mixture was quenched by the addition of 10% NaOH solution, then the reaction mixture was filtered and the cake filter was concentrated under vacuum to give crude product. The reaction residue was used directly for the next step. Compound 16-5 (14.0 g, crude) was obtained as a white solid.

### LCMS: [M+H]⁺: 717.3

### Step 5: 3-(tritylthio)cyclobutanamine (16-6)

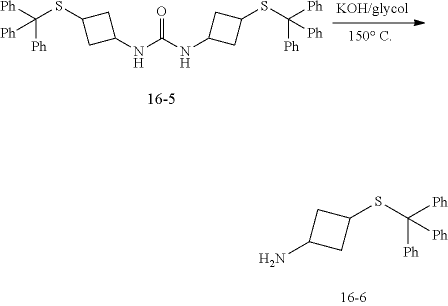

1,3-bis(3-tritylsulfanylcyclobutyl)urea (2.00 g, 2.79 mmol, 1 eq), KOH (313 mg, 5.58 mmol, 2 eq) were taken up into a microwave tube in ethylene glycol (10 mL). The sealed tube was heated at 150° C. for 1 hour under microwave. The reaction mixture was quenched by H₂O (30 mL) and extracted with ethyl acetate (30 mL×2). The combined organic phase was washed with brine (50 mL), dried with anhydrous Na₂SO₄, filtered and concentrated under vacuum to give a crude product. The reaction residue was used directly for the next step. Compound 16-6 (10.0 g, crude) was obtained as a brown oil.

### LCMS: [M+H]⁺: 346.1

### Step 6: N,N-dimethyl-3-(tritylthio)cyclobutanamine (16-7)

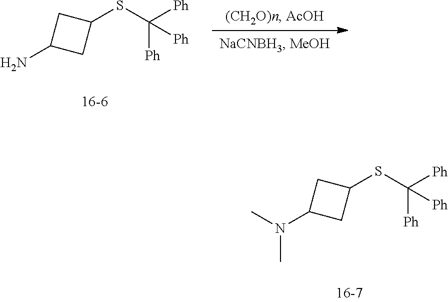 [SMILES: NC1CC(SC(c2ccccc2)(c2ccccc2)c2ccccc2)C1 ; "(CH2O)n, AcOH" ; CN(C)C1CC(SC(c2ccccc2)(c2ccccc2)c2ccccc2)C1]

To a mixture of 3-tritylsulfanylcyclobutanamine (10.0 g, 28.9 mmol, 1 eq) in MeOH (10 mL) were added (HCHO)ₙ (10.0 g, 145 mmol, 5 eq), AcOH (3.48 g, 57.9 mmol, 3.31 mL, 2 eq), NaBH₃CN (3.64 g, 57.9 mmol, 2 eq) successively at 0° C., then the reaction mixture was stirred at 25° C. for 3 hours. The reaction mixture was quenched by the addition of saturated NH₄Cl solution (20 mL) and extracted by ethyl acetate (30 mL×3), then the combined organic phase was dried by anhydrous sodium sulfate, filtered and concentrated under reduced pressure. The reaction residue was used directly for the next step. Compound 16-7 (6.00 g, crude) was obtained as a yellow oil.

### LCMS: [M+H]⁺: 374.3

### Step 7: 3-(dimethylamino)cyclobutanethiol (16-8)

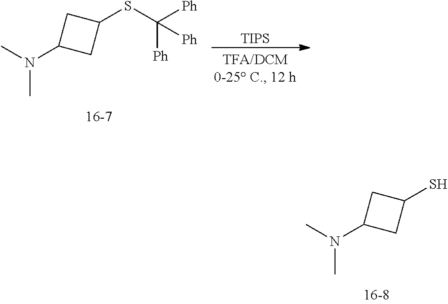

To a solution of N,N-dimethyl-3-tritylsulfanyl-cyclobutanamine (6.00 g, 16.1 mmol, 1 eq) in DCM (20 mL) were added triisopropylsilane (5.09 g, 32.1 mmol, 6.60 mL, 2 eq) and TFA (4.62 g, 40.5 mmol, 3 mL, 2.52 eq) at 0° C. The mixture was stirred at 25° C. for 12 hours. The reaction mixture was concentrated under reduced pressure to obtained a residue, then the reaction residue was added to MeOH (20 mL) and washed with Petroleum ether (3×10 mL), concentrated under vacuum. The reaction residue was used directly for the next step. Compound 16-8 (1.40 g, crude) was obtained as a yellow oil.

### Step 8: di(pentadecan-8-yl) 4,4′-((((3-(dimethylamino)cyclobutyl)thio)carbonyl)azanediyl)dibutanoate (CAT16)

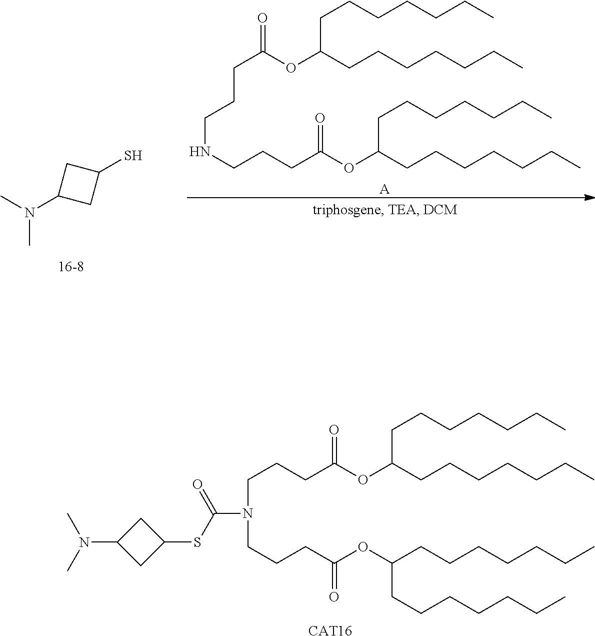

To a solution of 1-heptyloctyl 4-[[4-(1-heptyloctoxy)-4-oxo-butyl]amino]butanoate (2.00 g, 3.28 mmol, 1 eq) dissolved in dry DCM (30 mL) were added TEA (995 mg, 9.84 mmol, 1.37 mL, 3 eq) and bis(trichloromethyl) carbonate (486 mg, 1.64 mmol, 0.5 eq) at 0° C. under N₂. The resulting solution was stirred at 20° C. for 1 hour. The resulting reaction mixture was concentrated under reduced pressure and kept under N₂. To a solution of 3-(dimethylamino)cyclobutanethiol (1.72 g, 13.1 mmol, 4 eq) in dry THF (30 mL) was added NaOH (918 mg, 22.9 mmol, 7 eq) at 0° C. under N₂. To this resulting solution was added carbamoyl chloride via syringe slowly under N₂ at 0° C. The resulting solution was stirred at 20° C. for 1 hour. The reaction mixture was quenched with saturated aqueous NH₄C₁ (100 mL) and then diluted with ethyl acetate (100 mL). The aqueous phase was extracted with ethyl acetate (100 mL×3). The combined organic phase was washed with brine (100 mL), dried with anhydrous Na₂SO₄, filtered and concentrated under vacuum to give residue. The residue was purified by column chromatography (SiO₂, Petroleum ether/Ethyl acetate=50/1 to 3/1). Compound CAT16 (1.60 g, 2.09 mmol, 64% yield) was obtained as a yellow oil.

### LCMS: [M+H]⁺: 767.6

¹H NMR (400 MHz, CDCl₃) δ: 4.93-4.83 (m, 2H), 3.86-3.82 (m, 1H), 3.34-3.32 (m, 4H), 3.00-2.90 (m, 1H), 2.54-2.42 (m, 2H), 2.33-2.30 (m, 4H), 2.13 (s, 6H), 1.93-1.80 (m, 6H), 1.60-1.44 (m, 8H), 1.30-1.20 (m, 40H), 0.98-0.78 (m, 12H).

### Example 1.17: Synthesis of CAT17

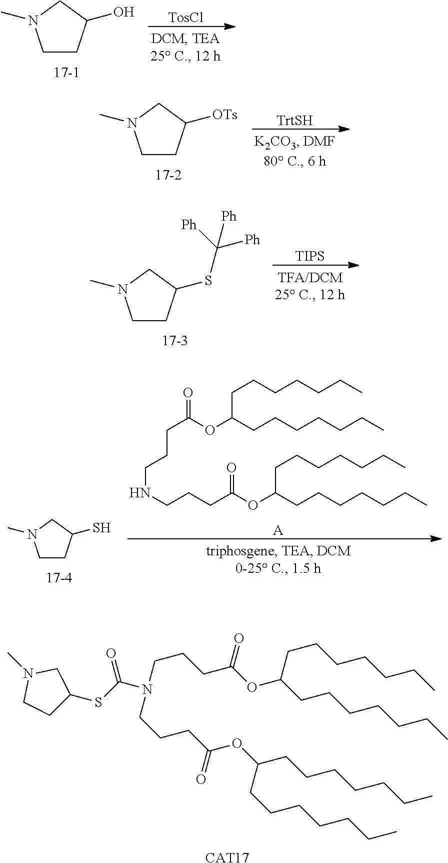

### Step 1: (1-methylpyrrolidin-3-yl) 4-methylbenzenesulfonate (17-2)

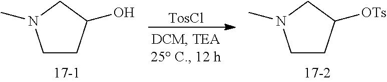

To a solution of 1-methylpyrrolidin-3-ol (20 g, 197.73 mmol, 1 eq) in CH₂Cl₂ (300 mL) was added TosCl (45.24 g, 237.28 mmol, 1.2 eq), TEA (60.03 g, 593.20 mmol, 82.57 mL, 3 eq) and DMAP (12.08 g, 98.87 mmol, 0.5 eq). The mixture was stirred at 25° C. for 12 hr. The reaction mixture was quenched with saturated aqueous water (300 mL) and then diluted with CH₂Cl₂ (100 mL). The aqueous phase was extracted with CH₂Cl₂ (100 mL×3). The combined organic phase was washed with brine (100 mL), dried with anhydrous Na₂SO₄, filtered and concentrated in vacuum to give a residue. The residue was purified by silica gel chromatography (Petroleum ether/Ethyl acetate=10/1 to 1/1) to give compound 17-2 (39 g, 152.74 mmol, 77.25% yield) as a yellow oil.

¹H NMR (400 MHz, CDCl₃) δ=7.79 (d, J=8.0 Hz, 2H), 7.34 (d, J=8.0 Hz, 2H), 5.10-4.96 (m, 1H), 2.70-2.58 (m, 2H), 2.44 (s, 3H), 2.38-2.32 (m, 2H), 2.31 (s, 3H), 2.18-2.12 (m, 1H), 1.95-1.90 (m, 1H)

### Step 2: 1-methyl-3-tritylsulfanyl-pyrrolidine: (17-3)

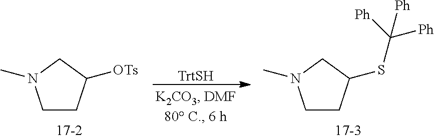 [SMILES: Cc1ccc(S(=O)(=O)OC2CCN(C)C2)cc1 ; "TrtSH" ; CN1CCC(SC(c2ccccc2)(c2ccccc2)c2ccccc2)C1]

To a solution of (1-methylpyrrolidin-3-yl) 4-methylbenzenesulfonate (15 g, 58.75 mmol, 1 eq) and triphenylmethanethiol (19.48 g, 70.50 mmol, 1.2 eq) in DMF (100 mL) was added K₂C₀₃ (24.36 g, 176.24 mmol, 3 eq). The mixture was stirred at 80° C. for 6 hr. The reaction mixture was quenched with saturated aqueous NH₄Cl (100 mL) and then diluted with EtOAc (300 mL). The aqueous phase was extracted with EtOAc (100 mL×3). The combined organic phase was washed with brine (200 mL), dried with anhydrous Na₂SO₄, filtered and concentrated in vacuum to give a residue. The residue was purified by silica gel chromatography (Petroleum ether/Ethyl acetate=10/1 to 0/1) to give compound 17-3(20 g, 55.63 mmol, 94.69% yield) as a yellow oil

¹H NMR (400 MHz, CDCl₃) δ=7.60-7.30 (m, 15H), 2.65-2.48 (m, 4H), 2.40-2.35 (m, 3H), 2.28-1.60 (m, 3H)

### Step 3: 1-methylpyrrolidine-3-thiol: (17-4)

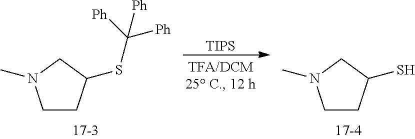

To a solution of 1-methyl-3-tritylsulfanyl-pyrrolidine (20 g, 55.63 mmol, 1 eq) in CH₂Cl₂ (300 mL) were added TFA (46.20 g, 405.18 mmol, 30.00 mL, 7.28 eq) and triisopropylsilane (26.43 g, 166.89 mmol, 34.28 mL, 3 eq). The mixture was stirred at 25° C. for 12 hr. The reaction mixture was concentrated under reduced pressure to remove TFA. The residue was diluted with MeOH (100 mL) and extracted with PE (50 mL×5). The MeOH layers was concentrated under reduced pressure to give compound 17-4 (5.4 g, 46.07 mmol, 82.82% yield) as a yellow oil.

### Step 4: 1-heptyloctyl 4-[[4-(1-heptyloctoxy)-4-oxo-butyl]-(1-methylpyrrolidin-3-yl)sulfanylcarbonyl-amino]butanoate: (CAT17)

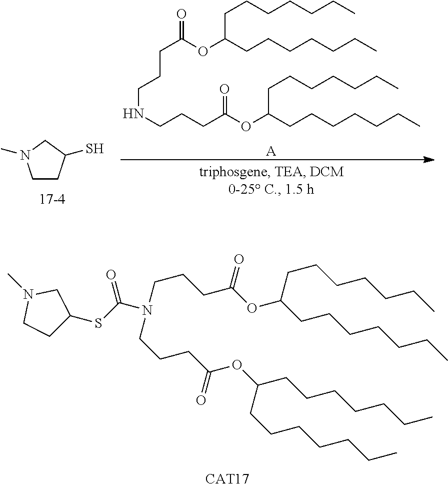

To a solution of 1-heptyloctyl 4-[[4-(1-heptyloctoxy)-4-oxo-butyl]amino]butanoate (1.5 g, 2.46 mmol, 1 eq) dissolved in dry CH₂Cl₂ (40 mL) were added TEA (746.47 mg, 7.38 mmol, 1.03 mL, 3 eq) and bis(trichloromethyl) carbonate (364.85 mg, 1.23 mmol, 0.5 eq) at 0° C. under nitrogen atmosphere. The resulting solution was stirred at 20° C. under nitrogen atmosphere for 1 hr. The reaction was concentrated under reduced pressure and kept under nitrogen atmosphere. NaOH (688.47 mg, 17.21 mmol, 7 eq) was dissolved in THF (50 mL) at 0° C. under nitrogen atmosphere, then 1-methylpyrrolidine-3-thiol (1.44 g, 12.30 mmol, 5 eq) was added under nitrogen atmosphere. To this resulting solution, carbamoyl chloride dissolved in THF (10 mL) was added slowly under nitrogen atmosphere at 0° C. The mixture was stirred at 25° C. for 0.5 hr. The reaction mixture was quenched with saturated aqueous NH₄C₁ (100 mL) and then diluted with EtOAc (200 mL). The aqueous phase was extracted with EtOAc (100 mL×3). The combined organic phase was washed with brine (100 mL), dried with anhydrous Na₂SO₄, filtered and concentrated in vacuum to give a residue. The residue was purified by silica gel chromatography (Petroleum ether/Ethyl acetate=10/1 to 0/1 and purified by column: Welch Ultimate XB-SiOH 250*50*10 μm; mobile phase: [Hexane-EtOH]; B %: 0%-13%, 10 min to give CAT17 (395 mg, 516.03 μmol, 64.78% yield, 98.4% purity) as a yellow oil.

### LCMS: [M+H]⁺: 753.8

¹H NMR (400 MHz, CDCl₃) δ=4.90-4.80 (m, 2H), 4.00-3.90 (m, 1H), 3.50-3.38 (m, 4H), 3.05-2.90 (m, 1H), 2.82-2.75 (m, 1H), 2.68-2.60 (m, 1H), 2.48-2.40 (m, 2H), 2.37 (s, 3H), 2.30 (t, J=7.2 Hz, 4H), 1.98-1.70 (m, 5H), 1.52-1.48 (m, 8H), 1.32-1.24 (m, 40H), 0.92-0.86 (m, 12H)

### Example 1.18: Synthesis of CAT18

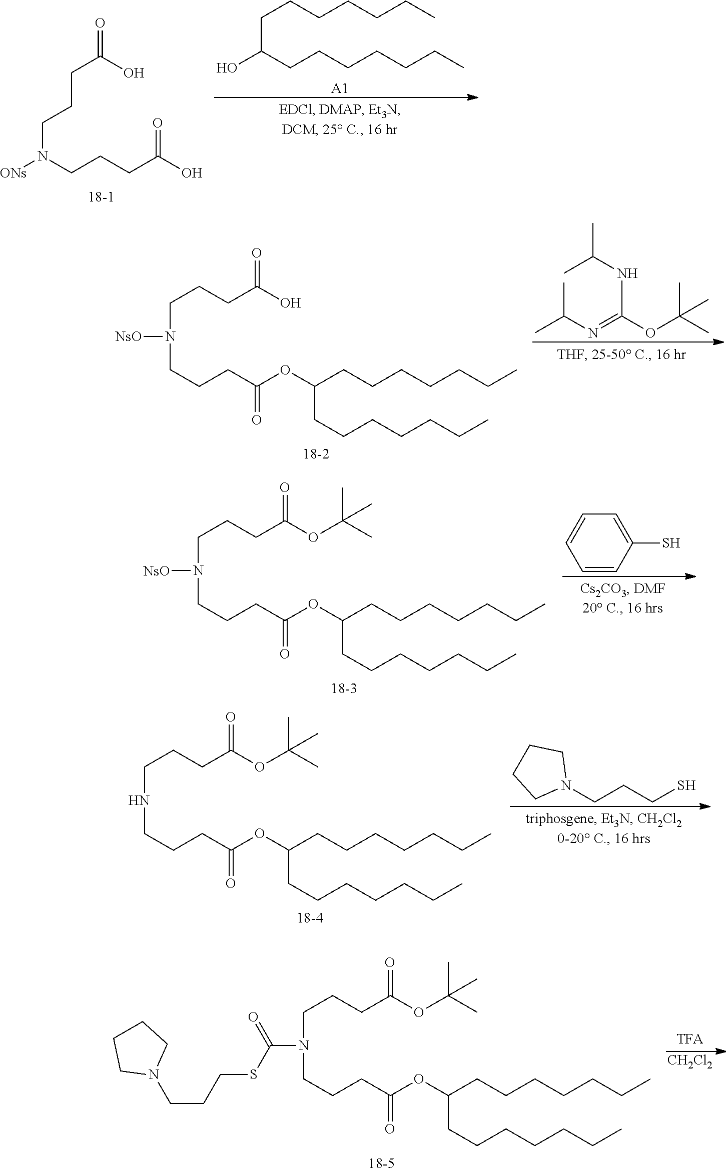

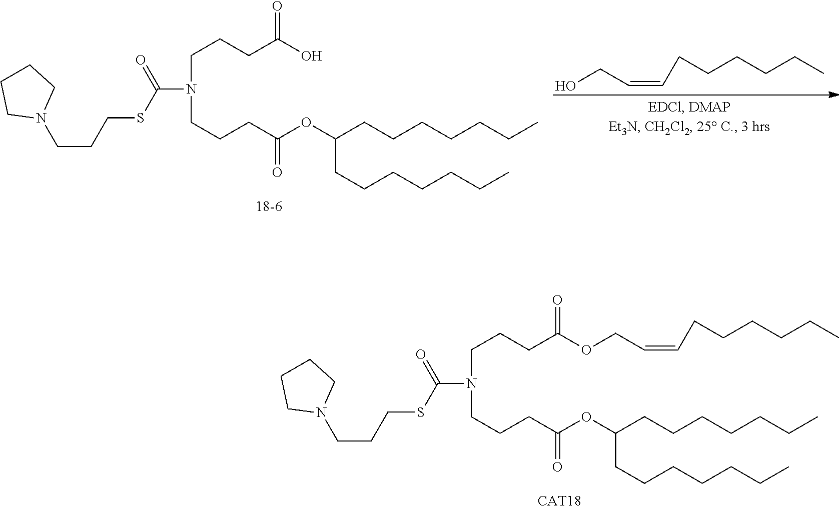

### Step 1: 4-(4-nitro-N-(4-oxo-4-(pentadecan-8-yloxy)butyl)phenylsulfonamido)butanoic acid (18-2)

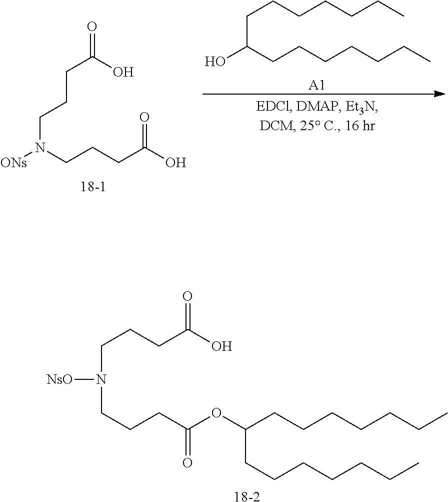

A mixture of 4-[3-carboxypropyl-(4-nitrophenyl)sulfonyl-amino]butanoic acid (25 g, 66.78 mmol, 1.04 eq), pentadecan-8-ol (8.09 g, 35.40 mmol, 0.55 eq), EDCI (6.79 g, 35.40 mmol, 0.55 eq), DMAP (786.38 mg, 6.44 mmol, 0.1 eq) and DIPEA (4.99 g, 38.62 mmol, 6.7 mL, 0.6 eq) in CH₂Cl₂ (200 mL) was degassed and purged with N₂ 3 times, and then the mixture was stirred at 25° C. for 16 hr under N₂ atmosphere. After completion, the reaction mixture was quenched by H₂O (150 mL) and then diluted with EtOAc (150 mL). The aqueous phase was extracted with EtOAc (150 mL*3). The combined organic phase was washed with brine (200 mL), dried with anhydrous Na₂SO₄, filtered and concentrated in vacuum to give residue. The residue was purified by flash silica gel chromatography (80 g SepaFlash®Silica Flash Column, EtOAc/PE: 0˜10%) to give compound 18-2 (12.8 g, 21.89 mmol, 34.0% yield) as a yellow solid.

¹H NMR (400 MHz, CDCl₃) δ=8.37-8.35 (m, 2H), 8.02-7.99 (m, 2H), 4.90-4.84 (m, 1H), 3.27-3.23 (m, 4H), 2.43 (t, J=7.2 Hz, 2H), 2.35 (t, J=7.2 Hz, 2H), 1.91-1.86 (m, 4H), 1.52-1.50 (m, 4H), 1.27-1.25 (m, 20H), 0.89-0.86 (m, 6H).

### Step 2: tert-butyl 4-(4-nitro-N-(4-oxo-4-(pentadecan-8-yloxy)butyl)phenylsulfonamido)butanoate (18-3)

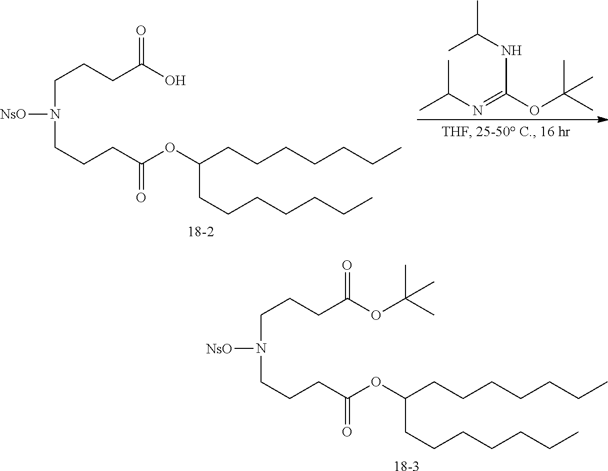

To a solution of 4-[[4-(1-heptyloctoxy)-4-oxo-butyl]-(4-nitrophenyl)sulfonyl-amino]butanoic acid (12.5 g, 21.38 mmol, 1 eq) in THF (150 mL) was added dropwise 2-tert-butyl-1,3-diisopropyl-isourea (12.85 g, 64.13 mmol, 3 eq) at 25° C. under N₂. After addition, the mixture was stirred at 50° C. for 16 hr. After addition, the reaction mixture was concentrated under reduced pressure to remove solvent to give a residue. The residue was purified by flash silica gel chromatography (80 g SepaFlash® Silica Flash Column, EtOAc/PE: 0˜10%) to give compound 3 (11.6 g, 18.10 mmol, 84.7% yield) as yellow oil.

¹H NMR (400 MHz, CDCl₃) δ=8.37-8.34 (m, 2H), 8.02-7.99 (m, 2H), 4.88-4.85 (m, 1H), 3.26-3.21 (m, 4H), 2.32 (t, J=7.2 Hz, 2H), 2.26 (t, J=7.2 Hz, 2H), 1.91-1.79 (m, 4H), 1.52-1.50 (m, 4H), 1.45 (s, 9H), 1.30-1.25 (m, 20H), 0.90-0.86 (m, 6H).

### Step 3: tert-butyl 4-((4-oxo-4-(pentadecan-8-yloxy)butyl)amino)butanoate (18-4)

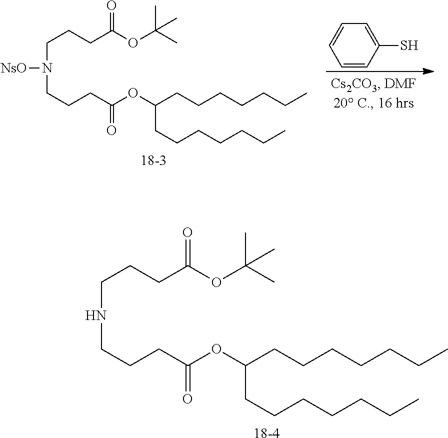

To a solution of 1-heptyloctyl 4-[(4-tert-butoxy-4-oxo-butyl)-(4-nitrophenyl)sulfonyl-amino]butanoate (8 g, 12.48 mmol, 1 eq) in DMF (50 mL) were added Cs₂CO₃ (8.13 g, 24.97 mmol, 2 eq) and benzenethiol (3.73 g, 33.85 mmol, 3.45 mL, 2.71 eq). The mixture was stirred at 25° C. for 16 hr under N₂. After completion, the reaction mixture was quenched by the addition of H2O (120 mL), and then extracted with EtOAc (150 mL*3). The combined organic layers were washed with brine (60 mL*3), dried over sodium sulfate, filtered and concentrated under reduced pressure. The residue was purified by flash silica gel chromatography (40 g SepaFlash® Silica Flash Column, EtOAc/PE: 0˜35%) to give compound 18-4 (4.1 g, 9.00 mmol, 72.1% yield) as yellow oil.

¹H NMR (400 MHz, CDCl₃) δ=4.90-4.84 (m, 1H), 2.65-2.61 (m, 4H), 2.35 (t, J=7.2 Hz, 2H), 2.27 (t, J=7.2 Hz, 2H), 1.80-1.76 (m, 4H), 1.52-1.48 (m, 4H), 1.44 (s, 9H), 1.30-1.26 (m, 20H), 0.90-0.86 (m, 6H).

### Step 4: tert-butyl 4-((4-oxo-4-(pentadecan-8-yloxy)butyl)(((3-(pyrrolidin-1-yl)propyl)thio)carbonyl)amino)butanoate (18-5)

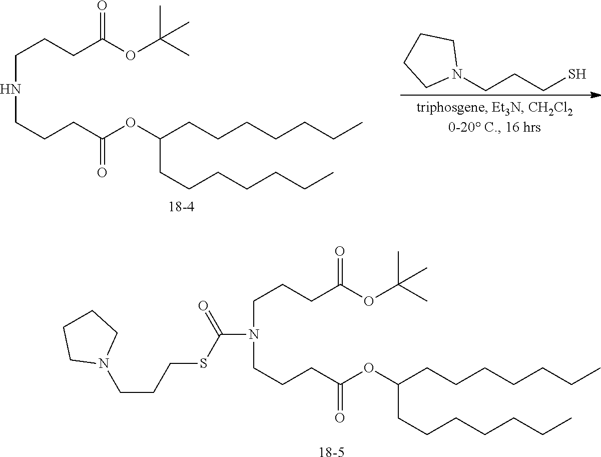

To a solution of 1-heptyloctyl 4-[(4-tert-butoxy-4-oxo-butyl)amino]butanoate (1.5 g, 3.29 mmol, 1 eq) dissolved in dry CH₂Cl₂ (30 mL) were added TEA (999.2 mg, 9.87 mmol, 1.4 mL, 3 eq) and triphosgene (586.1 mg, 1.97 mmol, 0.6 eq) at 0° C. under N₂. The resulting solution was stirred at 20° C. for 1 hour. The resulting reaction was concentrated under reduced pressure and kept under N₂. To a solution of 3-pyrrolidin-1-ylpropane-1-thiol (2.99 g, 11.52 mmol, 3.5 eq, TFA) dissolved in dry THF (30 mL) was added NaOH (921.63 mg, 23.04 mmol, 7 eq) at 0° C. under N₂. To this resulting solution, carbamoyl chloride, dissolved in THF (20 mL), was added via syringe slowly under N₂ at 0° C. The resulting solution was stirred at 20° C. for 15 hr. After completion, the reaction mixture was quenched by NH₄Cl (60 mL) at 0° C. and then diluted with EtOAc (50 mL). The aqueous phase was extracted with EtOAc (60 mL* 3). The combined organic phase was washed with brine (50 mL), dried with anhydrous Na₂SO₄, filtered and concentrated in vacuum to give a residue. The residue was purified by flash silica gel chromatography (20 g SepaFlash® Silica Flash Column, EtOAc: PE: 0˜13%, 5% NH₃·H₂O in EtOAc) to give compound 18-5 (1.05 g, 1.26 mmol, 41.5% yield, 75% purity) as a colorless oil.

¹H NMR (400 MHz, CDCl₃) δ=4.89-4.86 (m, 1H), 2.96-2.92 (m, 2H), 2.55-2.51 (m, 8H), 2.32-2.30 (m, 2H), 2.26-2.23 (m, 2H), 1.86-1.82 (m, 6H), 1.80-1.78 (m, 4H), 1.53-1.50 (m, 4H), 1.45 (s, 9H), 1.30-1.26 (m, 20H), 1.18-1.16 (m, 2H), 0.90-0.86 (m, 6H).

### Step 5: 4-((4-oxo-4-(pentadecan-8-yloxy)butyl)(((3-(pyrrolidin-1-yl)propyl)thio)carbonyl)amino)butanoic acid (18-6)

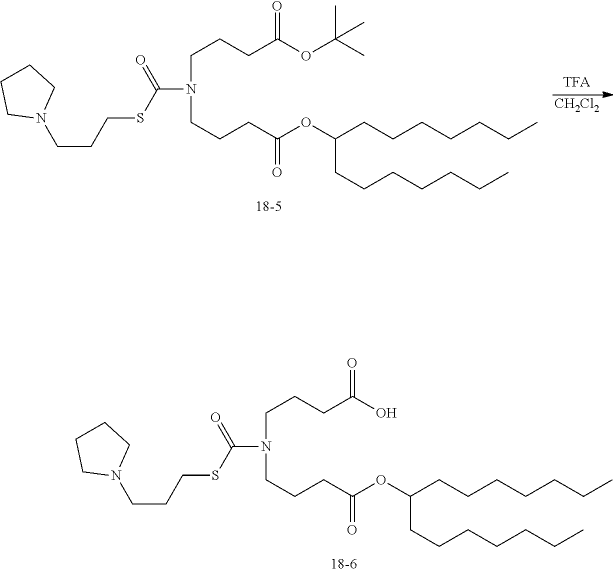

To a solution of 1-heptyloctyl 4-[(4-tert-butoxy-4-oxo-butyl)-(3-pyrrolidin-1-ylpropylsulfanylcarbonyl)amino]butanoate (950 mg, 1.52 mmol, 1 eq) in CH₂Cl₂ (10 mL) was added TFA (3.44 g, 30.19 mmol, 2.5 mL) under N₂. The mixture was stirred at 25° C. for 16 hr. After completion, the reaction mixture was concentrated under reduced pressure to remove solvent to give compound 18-6 (1.02 g, crude, TFA) as a yellow oil. The crude product was used in the next step without further purification.

### Step 6: (Z)-non-2-en-1-yl 4-((4-oxo-4-(pentadecan-8-yloxy)butyl)(((3-(pyrrolidin-1-yl)propyl)thio)carbonyl)amino)butanoate (CAT18)

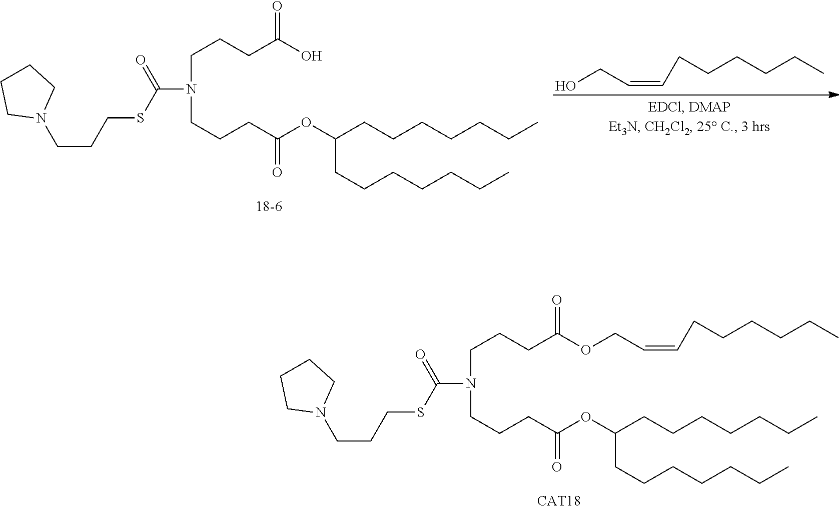

A mixture of 4-[[4-(1-heptyloctoxy)-4-oxo-butyl]-(3-pyrrolidin-1-ylpropylsulfanylcarbonyl)amino]butanoic acid (1.0 g, 1.46 mmol, 1 eq, TFA), (Z)-non-2-en-1-ol (415.4 mg, 2.92 mmol, 2 eq), EDCI (419.9 mg, 2.19 mmol, 1.5 eq), DMAP (17.8 mg, 0.15 mmol, 0.1 eq) and DIPEA (566.1 mg, 4.38 mmol, 0.76 mL, 3 eq) in CH₂Cl₂ (20 mL) was degassed and purged with N₂ 3 times, and then the mixture was stirred at 25° C. for 3 hr under N₂ atmosphere. After completion, the reaction mixture was quenched with H₂O (60 mL) and then diluted with EtOAc (50 mL). The aqueous phase was extracted with EtOAc (50 mL*3). The combined organic phase was washed with brine (60 mL), dried with anhydrous Na₂SO₄, filtered and concentrated in vacuum to give a residue. The residue was purified by flash silica gel chromatography (20 g SepaFlash® Silica Flash Column, EtOAc: PE: 0˜13%, 5% NH₃·H₂O in EtOAc) to yield compound CAT18 (413 mg, 0.58 mmol, 40.5% yield, 98.1% purity) as a light yellow oil.

### LCMS [M+H]⁺: 695.5

¹H NMR (400 MHz, CDCl₃) δ=5.68-5.62 (m, 1H), 5.54-5.51 (m, 1H), 4.89-4.86 (m, 1H), 4.64 (d, J=6.8 Hz, 2H), 3.39-3.37 (m, 4H), 2.94 (t, J=7.2 Hz, 2H), 2.52-2.50 (m, 6H), 2.36 -2.34 (m, 4H), 2.12-2.09 (m, 2H), 1.84-1.79 (m, 6H), 1.78-1.76 (m, 4H), 1.52-1.50 (m, 4H), 1.40-1.35 (m, 2H), 1.30-1.25 (m, 26H), 0.90-0.87 (m, 9H).

### Example 1.19: Synthesis of CAT19

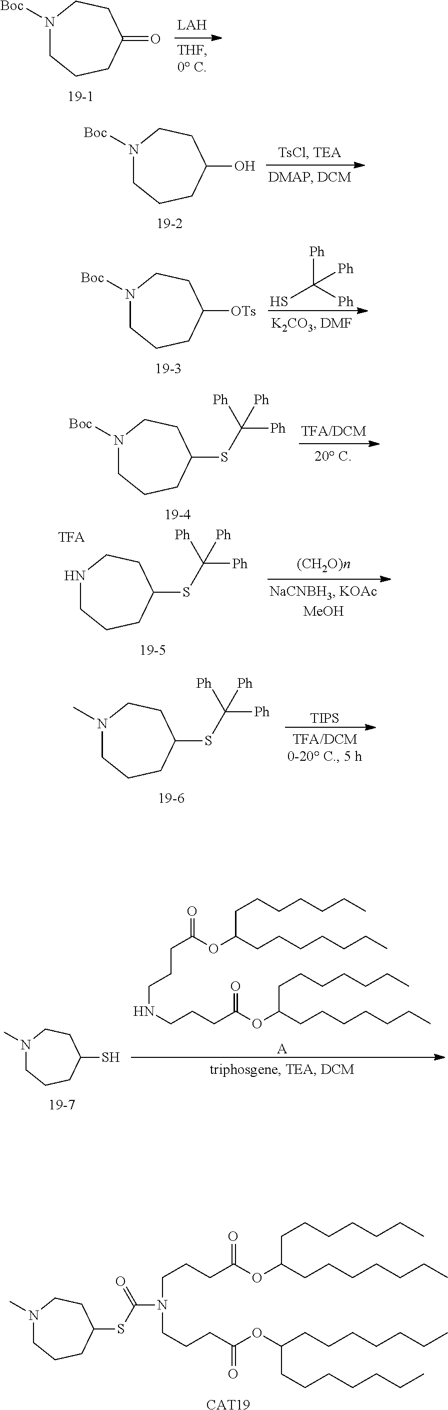

### Step 1: tert-butyl 4-hydroxyazepane-1-carboxylate (19-2)

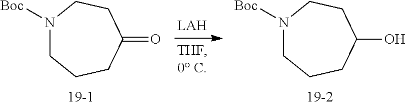 [SMILES: CC(C)(C)OC(=O)N1CCCC(=O)CC1 ; CC(C)(C)OC(=O)N1CCCC(O)CC1]

To a mixture of tert-butyl 4-oxoazepane-1-carboxylate (30.0 g, 141 mmol, 1 eq) in THF (300 mL) was added LiAlH₄ (5.87 g, 155 mmol, 1.1 eq) in portions at 0° C., then the reaction mixture was stirred at the same temperature for 2 h. The reaction mixture was quenched with H₂O (5.8 mL), aq.NaOH (17.4 mL, 4M), H₂O (5.8 mL) successively at 0° C. Then, anhydrous Na₂SO₄ (20.0 g) was added to the mixture which was stirred at the same temperature for 0.5 h. The reaction mixture was filtered and the filtrate was concentrated under vacuum to give a crude product. The reaction residue was used directly for the next step. Compound 19-2 (30.0 g, crude) was obtained as a yellow oil.

¹H NMR (400 MHz, DMSO-d₆) δ: 4.50 (t, J=4.0 Hz, 1H), 3.69-3.55 (m, 1H), 3.31-3.05 (m, 4H), 1.84-1.70 (m, 2H), 1.70-1.41 (m, 2H), 1.39 (s, 9H).

### Step 2: tert-butyl 4-(tosyloxy)azepane-1-carboxylate (19-3)

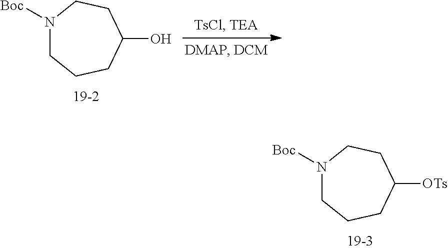 [SMILES: CC(C)(C)OC(=O)N1CCCC(O)CC1 ; Cc1ccc(S(=O)(=O)OC2CCCN(C(=O)OC(C)(C)C)CC2)cc1]

To a solution of tert-butyl 4-hydroxyazepane-1-carboxylate (30.0 g, 139 mmol, 1 eq) in DCM (500 mL) were added TEA (42.3 g, 418 mmol, 58.2 mL, 3 eq), DMAP (8.51 g, 69.7 mmol, 0.5 eq) and TosCl (39.9 g, 209 mmol, 1.5 eq) successively, then the mixture was stirred at 25° C. for 5 h. The reaction mixture was quenched by the addition of water (100 mL) and extracted with DCM (100 mL×3). The combined organic phase was washed with brine (100 mL), dried with anhydrous Na₂SO₄, filtered and concentrated under vacuum to give a residue. The residue was purified by flash silica gel chromatography (ISCO®; 220 g SepaFlash® Silica Flash Column, Eluent of 0˜20% Ethyl acetate/Petroleum ethergradient @ 100 mL/min). Compound 19-3(43.0 g, 116 mmol, 84% yield) was obtained as a brown oil.

### LCMS: [M-Boc]*: 270.0;

¹H NMR (400 MHz, CDCl₃) δ: 7.80-7.75 (m, 2H), 7.35-7.32 (m, 2H), 4.75-4.59 (m, 1H), 3.60-3.36 (m, 2H), 3.31-3.23 (m, 2H), 2.45 (s, 3H), 1.93-1.81 (m, 4H), 1.78-1.65 (m, 2H), 1.43 (s, 9H).

### Step 3: tert-butyl 4-(tritylthio)azepane-1-carboxylate (19-4)

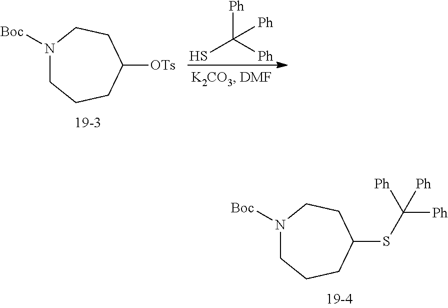 [SMILES: Cc1ccc(S(=O)(=O)OC2CCCN(C(=O)OC(C)(C)C)CC2)cc1 ; SC(c1ccccc1)(c1ccccc1)c1ccccc1 ; CC(C)(C)OC(=O)N1CCCC(SC(c2ccccc2)(c2ccccc2)c2ccccc2)CC1]

To a solution of tert-butyl 4-(p-tolylsulfonyloxy)azepane-1-carboxylate (21.0 g, 56.8 mmol, 1 eq) and triphenylmethanethiol (20.4 g, 73.9 mmol, 1.3 eq) in DMF (200 mL) was added Cs₂CO₃ (37.0 g, 114 mmol, 2 eq). The mixture was stirred at 80° C. for 6 h. The reaction mixture was quenched by the addition of water (100 mL) and extracted with ethyl acetate (150 mL×3). The combined organic phase was washed with brine (200 mL), dried with anhydrous Na₂SO₄, filtered and concentrated under vacuum to give residue. The residue was purified by flash silica gel chromatography (ISCO®; 330 g SepaFlash® Silica Flash Column, Eluent of 0 30% Ethyl acetate/Petroleum ethergradient @ 100 mL/min). Compound 19-4(21.0 g, 44.3 mmol, 39% yield) was obtained as a yellow oil.

### LCMS: [M+Na]⁺: 496.2

### Step 4: 4-(tritylthio)azepane (19-5)

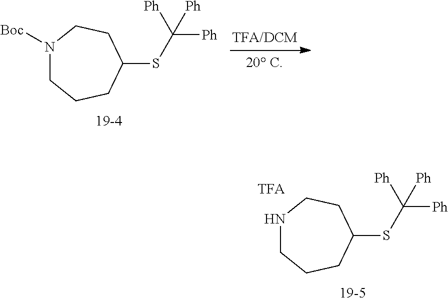 [SMILES: CC(C)(C)OC(=O)N1CCCC(SC(c2ccccc2)(c2ccccc2)c2ccccc2)CC1 ; c1ccc(C(SC2CCCNCC2)(c2ccccc2)c2ccccc2)cc1 ; "TFA"]

To a solution of tert-butyl 4-tritylsulfanylazepane-1-carboxylate (20.0 g, 42.2 mmol, 1 eq) in DCM (200 mL) was added TFA (61.6 g, 540 mmol, 40.0 mL, 12.8 eq), the reaction mixture was stirred at 20° C. for 3 h. The reaction mixture was concentrated under vacuum to obtain a brown oil. The reaction residue was used directly for the next step. Compound 19-5 (27.0 g, crude, TFA) was obtained as a brown oil.

### LCMS: [M+H]⁺: 374.1;

### Step 5: 3-(tritylthio)cyclobutanamine (19-6)

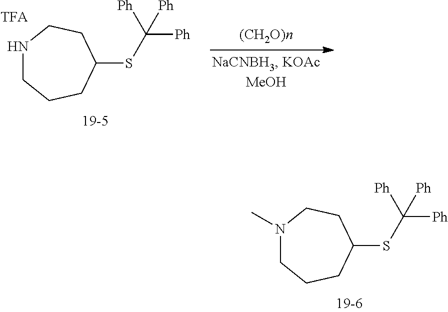 [SMILES: c1ccc(C(SC2CCCNCC2)(c2ccccc2)c2ccccc2)cc1 ; "TFA" ; CN1CCCC(SC(c2ccccc2)(c2ccccc2)c2ccccc2)CC1]

To a mixture of 4-tritylsulfanylazepane (10.0 g, 20.5 mmol, 1 eq, TFA) in MeOH (60 mL) were added (HCHO)ₙ (10.0 g, 20.5 mmol, 1 eq), KOAc (3.02 g, 30.8 mmol, 1.5 eq) and NaBH₃CN (2.58 g, 41.0 mmol, 2 eq) at 0° C. successively, then the reaction was stirred at 20° C. for 3 h. The reaction mixture was quenched by the addition of saturated NH₄Cl solution (20 mL) and extracted by ethyl acetate (30 mL×3), then the combined organic phase was dried by anhydrous sodium sulfate, filtered and concentrated under reduced pressure. The residue was purified by flash silica gel chromatography (ISCO®; 80 g SepaFlash® Silica Flash Column, Eluent of 0˜20% Dichloromethane: Methanol gradient @ 80 mL/min). Compound 19-6 (4.30 g, 11.1 mmol, 54% yield) was obtained as a yellow oil.

### LCMS: [M+H]⁺: 388.2;

### Step 6: 1-methylazepane-4-thiol (19-7)

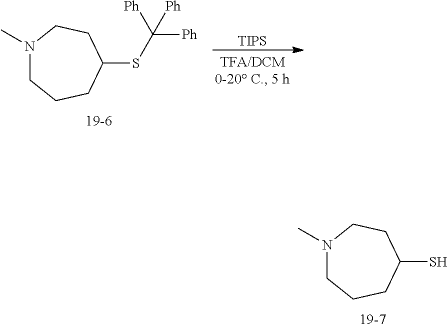

To a solution of 1-methyl-4-tritylsulfanyl-azepane (4.30 g, 11.1 mmol, 1 eq) in DCM (40 mL) were added triisopropylsilane (3.51 g, 22.2 mmol, 4.56 mL, 2 eq) and TFA (9.93 g, 87.1 mmol, 6.45 mL, 7.85 eq) at 0° C. The mixture was stirred at 25° C. for 5 h. The reaction mixture was concentrated under reduced pressure to obtain a residue, then the reaction residue was added to MeOH (20 mL) and washed with Petroleum ether three times (3×10 mL), concentrated under vacuum. The reaction residue was used directly for the next step. Compound 19-7 (2.10 g, crude) was obtained as a brown oil.

### Step 7: di(pentadecan-8-yl) 4,4′-((((1-methylazepan-4-yl)thio)carbonyl)azanediyl)dibutanoate (CA T19)

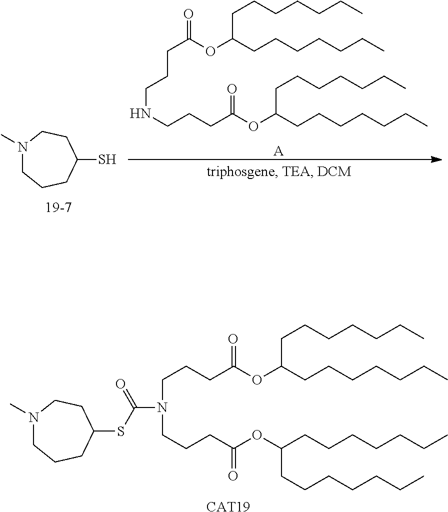

To a solution of 1-heptyloctyl 4-[[4-(1-heptyloctoxy)-4-oxo-butyl]amino]butanoate (1.50 g, 2.46 mmol, 1 eq) dissolved in dry DCM (30 mL) were added TEA (a746 mg, 7.38 mmol, 1.03 mL, 3 eq) and bis(trichloromethyl) carbonate (320 mg, 1.08 mmol, 4.39e-1 eq) at 0° C. under N₂. The resulting solution was stirred at 20° C. for 1 h. The resulting reaction was concentrated under reduced pressure and kept under N₂. To a solution of 1-methylazepane-4-thiol (1.43 g, 9.84 mmol, 4 eq) in dry THF (30 mL) was added NaOH (688 mg, 17.2 mmol, 7 eq) at 0° C. under N₂. To this resulting solution was added carbamoyl chloride via syringe slowly under N₂ at 0° C. The resulting solution was stirred at 20° C. for 1 h. The reaction mixture was quenched with saturated aqueous NH₄C₁ (100 mL) and then diluted with ethyl acetate (100 mL). The aqueous phase was extracted with ethyl acetate (100 mL×3). The combined organic phase was washed with brine (100 mL), dried with anhydrous Na₂SO₄, filtered and concentrated under vacuum to give residue. The residue was purified by column chromatography (SiO₂, Petroleum ether/Ethyl acetate=10/1 to 2/1). Compound CAT19 (700 mg, 0.896 mmol, 36% yield) was obtained as a yellow oil.

### LCMS: [M+H]⁺: 781.5;

¹H NMR (400 MHz, CDCl₃) δ: 4.90-4.84 (m, 2H), 3.79-3.62 (m, 1H), 3.46-3.27 (m, 4H), 2.77-2.48 (m, 4H), 2.36 (s, 3H), 2.33-2.29 (m, 4H), 2.20-2.06 (m, 2H), 1.94-1.86 (m, 4H), 1.82-1.74 (m, 2H), 1.68-1.63 (m, 2H), 1.53-1.50 (m, 8H), 1.33-1.22 (m, 40H), 0.95-0.86 (m, 12H).

### Example 1.20: Synthesis of CAT20

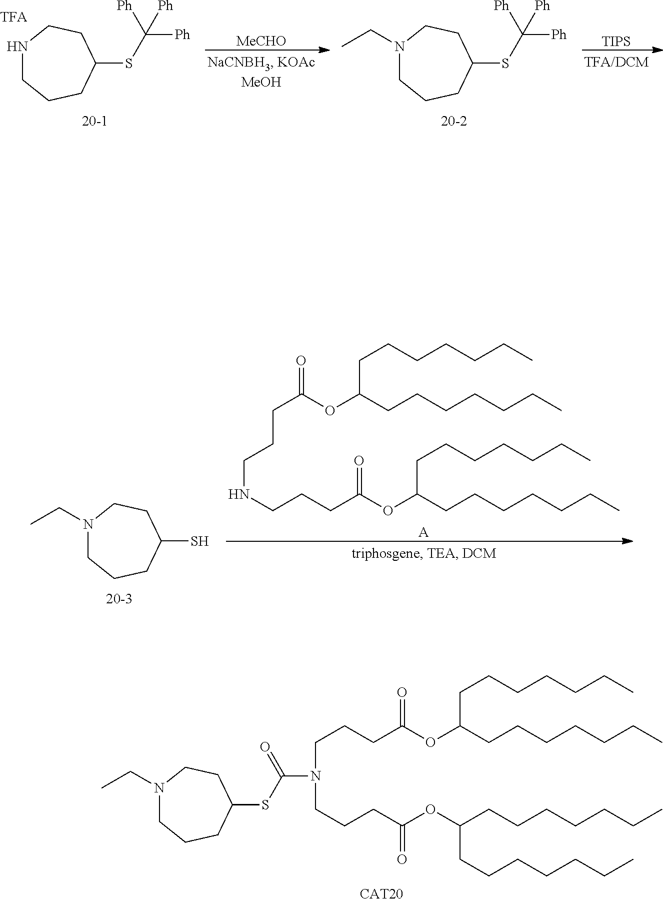

### Step 1: I-ethyl-4-(tritylthio)azepane (20-2)

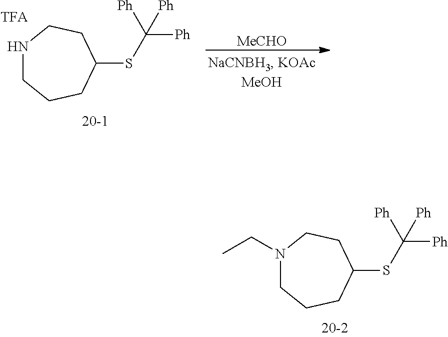 [SMILES: "MeCHO" ; c1ccc(C(SC2CCCNCC2)(c2ccccc2)c2ccccc2)cc1 ; "TFA" ; CCN1CCCC(SC(c2ccccc2)(c2ccccc2)c2ccccc2)CC1]

To a mixture of 4-tritylsulfanylazepane (10.0 g, 20.5 mmol, 1 eq, TFA) in MeOH (10 mL) were added KOAc (3.02 g, 30.8 mmol, 1.5 eq), MeCHO (4.52 g, 41.0 mmol, 5.75 mL, 40% purity, 2 eq) and NaBH₃CN (2.58 g, 41.0 mmol, 2 eq) successively, then the reaction mixture was stirred at 20° C. for 3 hours. The reaction mixture was quenched by the addition of saturated NH₄Cl solution (20 mL) and extracted by ethyl acetate (30 mL×3), then the combined organic phase was dried by anhydrous sodium sulfate, filtered and concentrated under reduced pressure. The residue was purified by column chromatography (SiO₂, Petroleum ether/Ethyl acetate=10/1 to 2/1). Compound 20-2(8.00 g, crude) was obtained as a yellow oil.

### LCMS: [M+H]⁺: 402.2

### Step 2: I-ethylazepane-4-thiol (20-3)

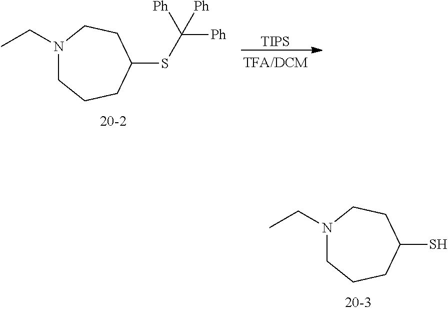

To a solution of 1-ethyl-4-tritylsulfanyl-azepane (8.00 g, 19.9 mmol, 1 eq) in DCM (40 mL) were added triisopropylsilane (6.31 g, 39.8 mmol, 8.18 mL, 2 eq) and TFA (17.8 g, 156 mmol, 11.6 mL, 7.85 eq) at 0° C. The mixture was stirred at 25° C. for 12 hours. The reaction mixture was concentrated under reduced pressure to obtained a residue, then the reaction residue was added to MeOH (20 mL) and washed with Petroleum ether three times (3×10 mL), and concentrated under vacuum. The reaction residue was used directly for the next step. Compound 20-3 (2.30 g, crude) was obtained as a brown oil.

### Step 3: di(pentadecan-8-yl) 4,4′-((((1-ethylazepan-4-yl)thio)carbonyl)azanediyl)dibutanoate (CAT20)

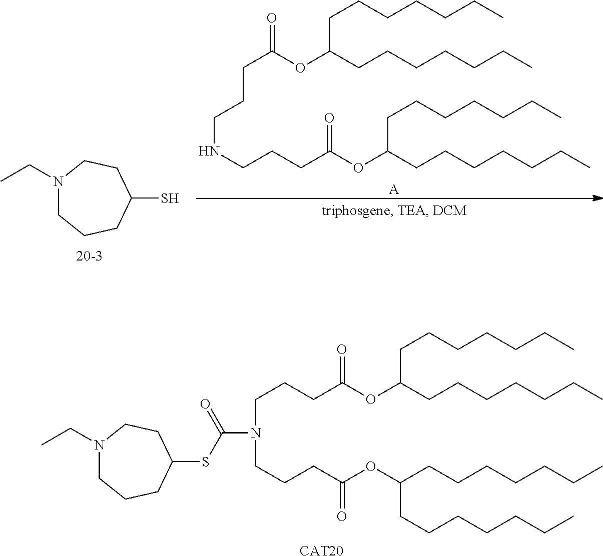

To a solution of 1-heptyloctyl 4-[[4-(1-heptyloctoxy)-4-oxo-butyl]amino]butanoate (2.00 g, 3.28 mmol, 1 eq) dissolved in dry DCM (30 mL) were added TEA (995 mg, 9.84 mmol, 1.37 mL, 3 eq) and bis(trichloromethyl) carbonate (300 mg, 1.01 mmol, 3.08e-1 eq) at 0° C. under N₂. The resulting solution was stirred at 20° C. for 1 hour. The resulting reaction was concentrated under reduced pressure and kept under N₂. To a solution of 1-ethylazepane-4-thiol (2.09 g, 13.1 mmol, 4 eq) in dry THF (30 mL) was added NaOH (918 mg, 23.0 mmol, 7 eq) at 0° C. under N₂. To this resulting solution was added carbamoyl chloride via syringe slowly under N₂ at 0° C. The resulting solution was stirred at 20° C. for 1 hour. The reaction mixture was quenched with saturated aqueous NH₄C₁ (100 mL) and then diluted with ethyl acetate (100 mL). The aqueous phase was extracted with ethyl acetate (100 mL×3). The combined organic phase was washed with brine (100 mL), dried with anhydrous Na₂SO₄, filtered and concentrated under vacuum to give a residue. The residue was purified by column chromatography (SiO₂, Petroleum ether/Ethyl acetate=10/1 to 2/1). Compound CAT20 (1.80 g, 2.26 mmol, 69% yield) was obtained as a yellow oil.

### LCMS: [M+H]⁺: 796.4;

¹H NMR (400 MHz, CDCl₃) δ: 4.90-4.84 (m, 2H), 3.70-3.63 (m, 1H), 3.44-3.29 (m, 4H), 2.84-2.70 (m, 2H), 2.62-2.52 (m, 2H), 2.34-2.27 (m, 4H), 2.18-2.07 (m, 2H), 1.96-1.73 (m, 1OH), 1.53-1.50 (m, 8H), 1.32-1.25 (m, 40H), 1.08 (t, J=7.2 Hz, 3H), 0.91-0.86 (m, 12H).

### Example 1.21: Synthesis of CAT21

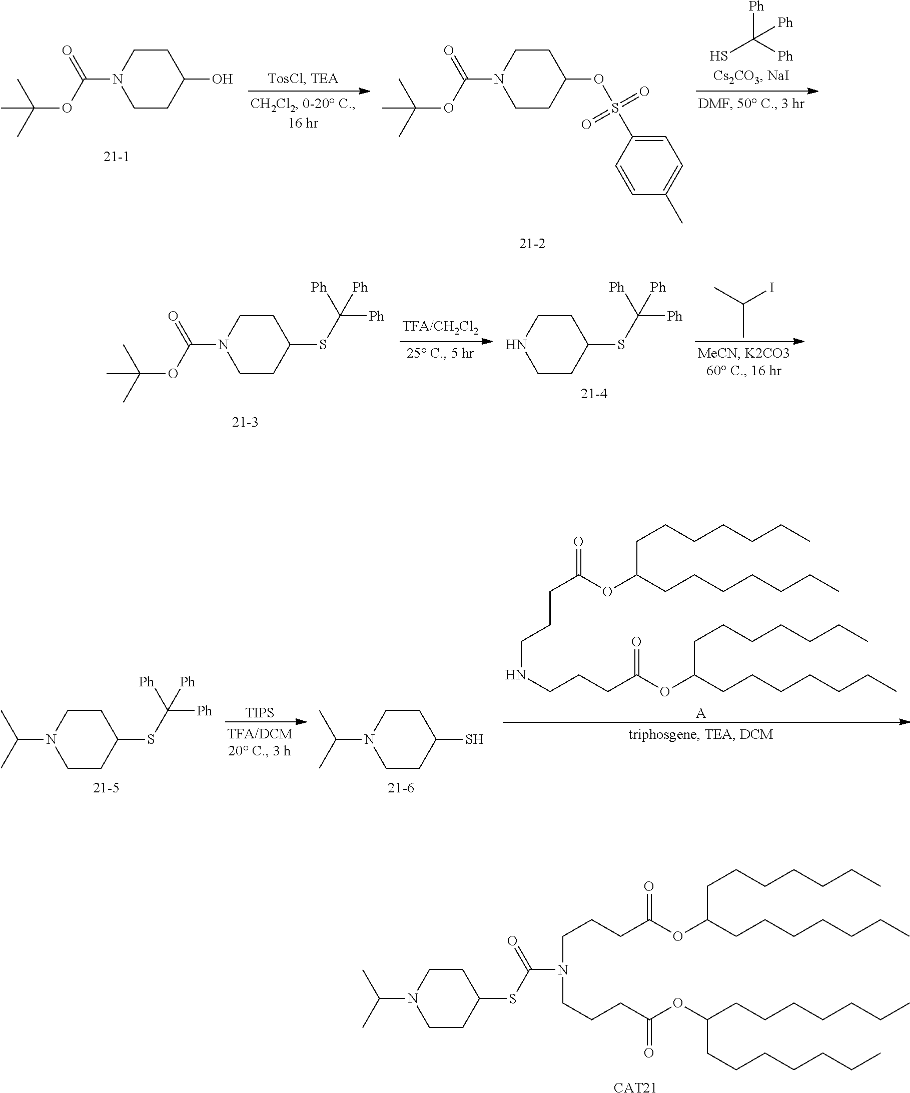

### Step 1: tert-butyl 4-(tosyloxy)piperidine-1-carboxylate (21-2)

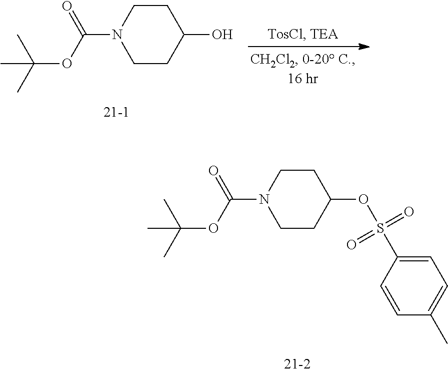 [SMILES: CC(C)(C)OC(=O)N1CCC(O)CC1 ; Cc1ccc(S(=O)(=O)OC2CCN(C(=O)OC(C)(C)C)CC2)cc1]

To a solution of tert-butyl 4-hydroxypiperidine-1-carboxylate (50 g, 248.43 mmol, 1 eq) in CH₂Cl₂ (500 mL) were added TEA (50.28 g, 496.86 mmol, 69.2 mL, 2 eq), DMAP (1.52 g, 12.42 mmol, 0.05 eq) and TosCl (71.04 g, 372.65 mmol, 1.5 eq) at 0° C. under N₂. The mixture was stirred at 20° C. for 16 hr. After completion, the reaction mixture was diluted with CH₂Cl₂ (300 mL) and washed with brine (300 mL*3), dried with anhydrous Na₂SO₄, filtered and concentrated in vacuum to give residue. The residue was purified by flash silica gel chromatography (220 g SepaFlash® Silica Flash Column, EtOAc: PE: 0˜25%) to give compound 21-2 (82.6 g, 232.38 mmol, 91.8% yield) as a yellow solid.

¹H NMR (400 MHz, CDCl₃) δ=7.80 (d, J=8.4 Hz, 2H), 7.34 (d, J=8.0 Hz, 2H), 4.70-4.65 (m, 1H), 3.59-3.57 (m, 2H), 3.28-3.23 (m, 2H), 2.45 (s, 3H), 1.77-1.74 (m, 2H), 1.70-1.67 (m, 2H), 1.43 (s, 9H).

### Step 2: tert-butyl 4-(tritylthio)piperidine-1-carboxylate (21-3)

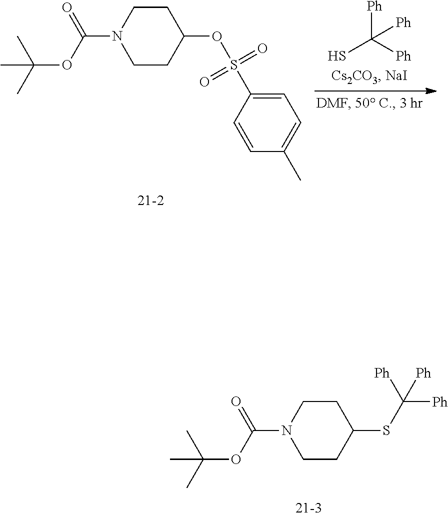

A mixture of tert-butyl 4-(p-tolylsulfonyloxy)piperidine-1-carboxylate (40 g, 112.53 mmol, 1 eq), triphenylmethanethiol (37.32 g, 135.04 mmol, 1.2 eq), NaI (843.39 mg, 5.63 mmol, 0.05 eq), Cs₂CO₃ (55.00 g, 168.80 mmol, 1.5 eq) in DMF (300 mL) was degassed and purged with N₂ 3 times, and then the mixture was stirred at 50° C. for 3 hr under N₂ atmosphere. After the addition, the reaction mixture was quenched with H₂O (600 mL) and then diluted with EtOAc (500 mL). The aqueous phase was extracted with EtOAc (500 mL*3). The combined organic phase was washed with brine (500 mL*3), dried with anhydrous Na₂SO₄, filtered and concentrated in vacuum to give a crude product. The crude product was purified by flash silica gel chromatography (330 g SepaFlash® Silica Flash Column, EtOAc: PE: 0˜5%) to give compound 21-3 (75.8 g, 164.91 mmol, 75.1% yield) as a yellow oil.

¹H NMR (400 MHz, CD30D-d₄) δ=7.33-7.31 (m, 5H), 7.29-7.26 (m, 1OH), 3.70-3.67 (m, 2H), 2.69-2.64 (m, 2H), 2.40-2.35 (m, 1H), 1.57-1.48 (m, 2H), 1.45 (s, 9H), 1.42-1.34 (m, 2H).

### Step 3: 4-(tritylthio)piperidine (21-4)

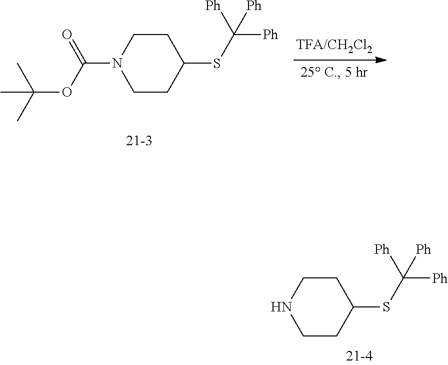 [SMILES: CC(C)(C)OC(=O)N1CCC(SC(c2ccccc2)(c2ccccc2)c2ccccc2)CC1 ; c1ccc(C(SC2CCNCC2)(c2ccccc2)c2ccccc2)cc1]

To a solution of tert-butyl 4-tritylsulfanylpiperidine-1-carboxylate (75 g, 163.17 mmol, 1 eq) in DCM (500 mL) was added TFA (154.00 g, 1.35 mol, 100 mL, 8.28 eq) at 25° C. under N₂. After addition, the mixture was stirred at 25° C. for 5 hr. After completion, the mixture was concentrated in vacuo. Most of the TFA was removed by rotary evaporation, and the residual TFA was co-evaporated with MeOH. The residue was triturated with PE (500 mL) at 25° C. for 0.5 hr. The residue mixture was filtered and the filter cake was washed with PE (100 mL*2). The filter cake was concentrated in vacuum to give compound 21-4 (56.8 g, crude, TFA) as a white solid.

¹H NMR (400 MHz, CDCl₃) δ=9.00-8.85 (m, 1H), 7.41-7.38 (m, 6H), 7.23-7.20 (m, 6H), 7.19-7.13 (m, 3H), 3.05-3.03 (m, 2H), 2.64-2.63 (m, 2H), 2.36-2.32 (m, 1H), 1.54-1.42 (m, 4H).

### Step 4: 1-isopropyl-4-(tritylthio)piperidine (21-5)

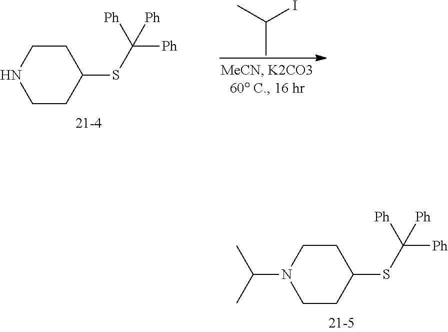

To a solution of 4-tritylsulfanylpiperidine (15 g, 31.68 mmol, 1 eq, TFA) in MeCN (150 mL) were added K₂CO₃ (13.13 g, 95.03 mmol, 3 eq) and 2-iodopropane (5.92 g, 34.84 mmol, 3.48 mL, 1.1 eq). The mixture was stirred at 60° C. for 16 hr. After completion, the reaction mixture was filtered and the filtrate was concentrated in a vacuum to give a residue. The residue was purified by flash silica gel chromatography (80 g SepaFlash® Silica Flash Column, MeOH/EtOAc: 0˜5%) to give compound 21-5 (8.2 g, 20.42 mmol, 64.46% yield) as a yellow oil.

¹H NMR (400 MHz, CDCl₃) δ=7.55-7.50 (m, 6H), 7.32-7.27 (m, 6H), 7.24-7.18 (m, 3H), 2.67-2.61 (m, 2H), 2.61-2.53 (m, 1H), 2.25-2.15 (m, 1H), 1.94 (t, J=9.0 Hz, 2H), 1.50-1.40 (m, 4H), 0.96 (d, J=6.4 Hz, 6H).

### Step 5: 1-isopropylpiperidine-4-thiol (21-6)

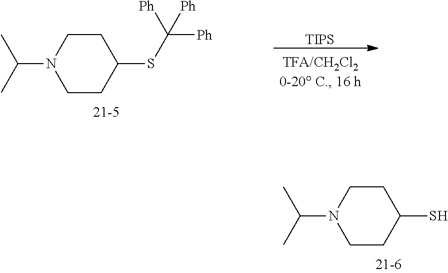

To a solution of 1-isopropyl-4-tritylsulfanyl-piperidine (8.1 g, 20.17 mmol, 1 eq) in CH₂Cl₂ (80 mL) were added TFA (30.80 g, 270.13 mmol, 20 mL, 13.39 eq) and TIPS (7.91 g, 40.34 mmol, 2 eq) at 0° C. under N₂. After addition, the resulting mixture was stirred at 20° C. for 16 hr. After completion, the reaction mixture was concentrated under reduced pressure to remove TFA and filtered. The filtrate was diluted with MeOH (150 mL) and extracted with PE (50 mL*5). The MeOH layers was concentrated under reduced pressure to give compound 21-6 (5.6 g, crude, TFA) as a yellow oil. The crude product was used in the next step without further purification.

### Step 6: ddi(pentadecan-8-yl) 4,4′-((((1-isopropylpiperidin-4-yl)thio)carbonyl)azanediyl)dibutanoate (CAT21)

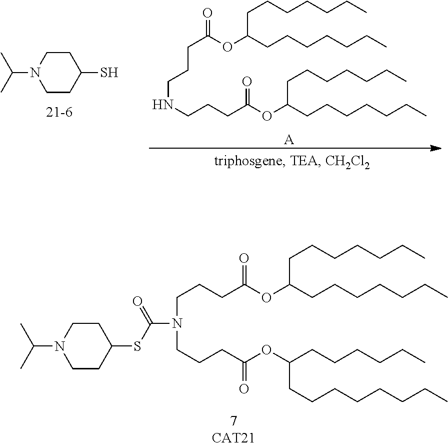

To a solution of 1-heptyloctyl 4-[[4-(1-heptyloctoxy)-4-oxo-butyl]amino]butanoate (2 g, 3.28 mmol, 1 eq) dissolved in dry CH₂Cl₂ (25 mL) were added TEA (995.31 mg, 9.84 mmol, 1.37 mL, 3 eq) and triphosgene (583.8 mg, 1.97 mmol, 0.6 eq) at 0° C. under N₂. The resulting solution was stirred at 20° C. for 1 hr. The resulting reaction was concentrated under reduced pressure. To a solution of 1-isopropylpiperidine-4-thiol (3.14 g, 11.48 mmol, 3.5 eq, TFA) dissolved in dry THF (30 mL) was added NaOH (918.03 mg, 22.95 mmol, 7 eq) at 0° C. under N₂. To this resulting solution, carbamoyl chloride, dissolved in THF (20 mL) was added via syringe slowly under N₂ at 0° C. The resulting solution was stirred at 20° C. for 15 hr. After completion, the reaction mixture was quenched by NH₄Cl (60 mL) at 0° C. and then diluted with EtOAc (50 mL). The aqueous phase was extracted with EtOAc (60 mL*3). The combined organic phase was washed with brine (50 mL), dried with anhydrous Na₂SO₄, filtered and concentrated in vacuum to give a residue. The residue was purified by flash silica gel chromatography (20 g SepaFlash® Silica Flash Column, EtOAc: PE: 0˜12%, 5% NH₃·H2O in Ethyl acetate) and purified by positive prep-HPLC (column: Welch Ultimate XB-NH2 250*50*10 um; mobile phase: [Hexane-EtOH];B %: 0%-20%, 15 min) to afford CAT21 (428 mg, 0.52 mmol, 57.7% yield, 97% purity) as a yellow oil.

### LCMS [M+H]⁺: 795.6;

¹H NMR (400 MHz, CDCl₃) δ=4.90-4.84 (m, 2H), 3.40-3.37 (m, 4H), 2.82-2.79 (m, 2H), 2.70-2.67 (m, 1H), 2.35-2.30 (m, 6H), 2.04-2.01 (m, 2H), 1.95-1.85 (m, 4H), 1.72-1.66 (m, 3H), 1.52-1.50 (m, 8H), 1.32-1.26 (m, 40H), 1.03 (d, J=6.4 Hz 6H), 0.90-0.86 (m, 12H).

### Example 1.22: Synthesis of CAT22

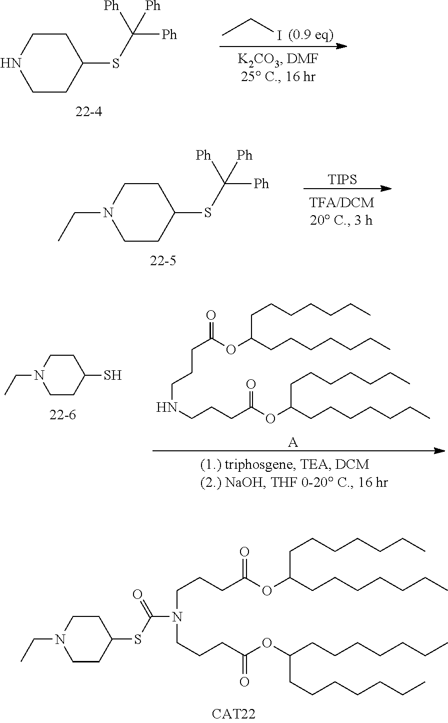

### Step 1: 1-ethyl-4-(tritylthio)piperidine (22-5)

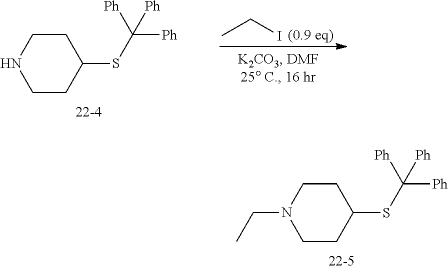

To a solution of 4-tritylsulfanylpiperidine (15.0 g, 31.9 mmol, 1.00 eq, TFA) in DMF (100 mL) were added K₂C₀₃ (13.1 g, 95.0 mmol, 3.00 eq) and iodoethane (4.45 g, 28.5 mmol, 2.28 mL, 0.90 eq). The mixture was stirred at 25° C. for 16 hours. The reaction mixture was quenched by water (150 mL) and then diluted with ethyl acetate (100 mL). The aqueous phase was extracted with ethyl acetate (150 mL×3). The combined organic phase was washed with brine (100 mL×3), dried with anhydroussodium sulfate, filtered and concentrated in vacuum to give a residue. The residue was purified by flash silica gel chromatography (80 g SepaFlash® Silica Flash Column, Ethyl acetate: Petroleumether: 0˜40%), then the residue was purified by inverted MPLC (MeCN: H₂O: 0˜40%) to give compound 22-5 (8.60 g, 22.0 mmol, 78.8% yield, 99% purity) as a yellow oil.

¹H NMR (400 MHz, MeOD-d₄) δ=7.53-7.50 (m, 6H), 7.35-7.31 (m, 6H), 7.28-7.23 (m, 3H), 3.39-3.32 (m, 2H), 3.16-3.00 (m, 3H), 2.70-2.63 (m, 2H), 2.48-2.40 (m, 1H), 1.87-1.63 (m, 2H), 1.57-1.47 (m, 2H), 1.33-1.24 (m, 3H).

### Step 2: 1-ethylpiperidine-4-thiol (22-6)

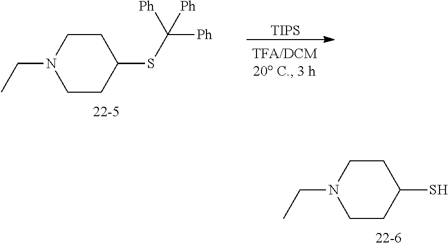

A mixture of 1-ethyl-4-tritylsulfanyl-piperidine (4.50 g, 11.6 mmol, 1.00 eq) in TFA (15.0 mL) and dichlormethane (50.0 mL), the mixture was degassed and purged with nitrogen atmosphere three times, then triisopropylsilane (3.68 g, 23.2 mmol, 4.77 mL, 2.00 eq) was added slowly at 0° C., and then the mixture was stirred at 20° C. for 3 hours under nitrogen atmosphere. The reaction mixture was concentrated under reduced pressure to remove TFA and filtered. The filtrate was diluted with methyl alcohol (50.0 mL) and extracted with petroleum ether (50.0 mL×5). The methyl alcohol layers was concentrated under reduced pressure to give a crude product to yield compound 22-6 (3.01 g, crude, TFA salt) as a yellow oil.

¹H NMR (400 MHz, DMSO-d₆) δ=3.43-3.40 (m, 2H), 3.18-3.10 (m, 1H), 3.08-2.97 (m, 2H), 2.94-2.87 (m, 2H), 2.08 (d, J=14 Hz, 2H), 1.84-1.73 (m, 2H), 1.24-1.18 (m, 3H).

### Step 3: di(pentadecan-8-yl) 4,4′-((((1-ethylpiperidin-4-yl)thio)carbonyl)azanediyl)dibutanoate (CAT22)

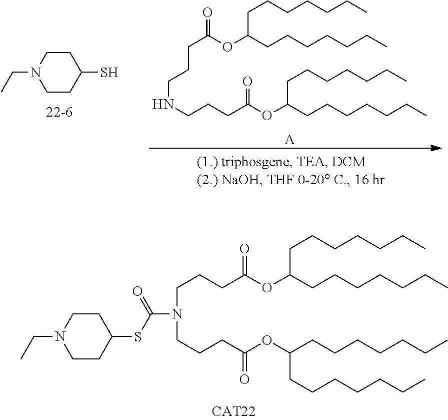

To a solution of 1-heptyloctyl 4-[[4-(1-heptyloctoxy)-4-oxo-butyl]amino]butanoate (2.00 g, 3.30 mmol, 1.00 eq) dissolved in dry dichloromethane (25.0 mL) were added TEA (995 mg, 9.80 mmol, 1.37 mL, 3.00 eq) and triphosgene (540 mg, 1.80 mmol, 0.50 eq) at 0° C. under N₂. The resulting solution was stirred at 20° C. for 1 hour. The resulting reaction was concentrated under reduced pressure. To 1-ethylpiperidine-4-thiol (2.98 g, 11.5 mmol, 3.50 eq, TFA salt) dissolved in dry THF (30.0 mL) was added NaOH (918 mg, 23.0 mmol, 7.00 eq) at 0° C. under nitrogen atmosphere. To this resulting solution, carbamoyl chloride, dissolved in THF (25.0 mL) was added via syringe slowly under N₂ at 0° C. The resulting solution was stirred at 20° C. for 15 hours. The reaction mixture was quenched by NH₄Cl (60.0 mL) at 0° C. and then diluted with ethyl acetate (60.0 mL). The aqueous phase was extracted with ethyl acetate (60.0 mL×3). The combined organic phase was washed with brine (100 mL), dried with anhydrous sodium sulfate, filtered and concentrated in vacuum to give a residue. The residue was purified by flash silica gel chromatography (ISCO®; 120 g SepaFlash® Silica Flash Column, Eluent of 0˜50% Ethyl acetate/Petroleum ethergradient @ 100 mL/min) to give compound CAT22 (268 mg, 0.34 mmol, 10.3% yield, 98.8% purity) as a light yellow oil.

### LCMS [M+1]⁺: 781.7;

¹H NMR (400 MHz, CDCl₃) δ=4.92-4.86 (m, 2H), 3.46-3.32 (m, 4H), 2.87-2.84 (m, 2H), 2.45-2.40 (m, 2H), 2.36-2.31 (m, 4H), 2.16 (t, J=9.6 Hz, 2H), 2.07-2.04 (m, 2H), 1.91 (s, 4H), 1.77-1.68 (m, 2H), 1.63-1.62 (m, 1H), 1.54-1.53 (m, 8H), 1.34-1.28 (m, 40H), 1.10 (t, J=7.2 Hz, 3H), 0.92-0.88 (m, 12H).

### Example 1.22: Synthesis of CAT23

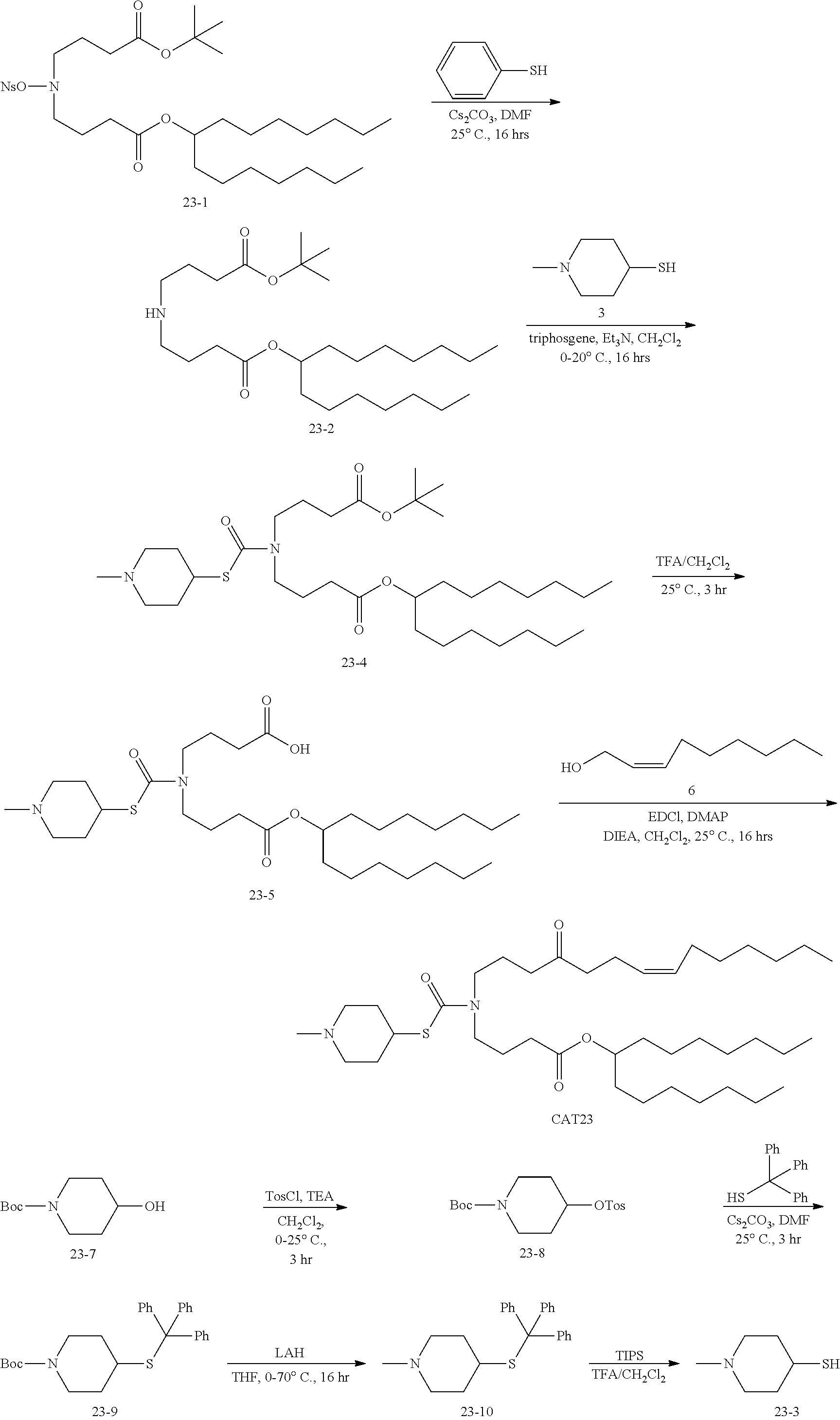

### Step 1: tert-butyl 4-(tosyloxy)piperidine-1-carboxylate (23-8)

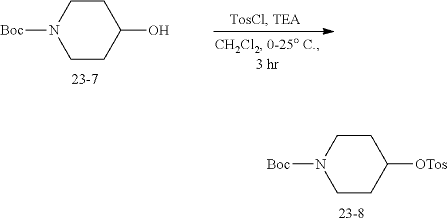

To a solution of tert-butyl 4-hydroxypiperidine-1-carboxylate (70 g, 347.81 mmol, 1 eq) in CH₂Cl₂ (750 mL) were added TEA (70.39 g, 695.61 mmol, 96.82 mL, 2 eq) and DMAP (2.12 g, 17.39 mmol, 0.05 eq) at 20° C. under N₂. After addition, the mixture was stirred at 20° C. for 0.5 hr, and then was added TosCl (79.57 g, 417.37 mmol, 1.2 eq) in portions at 0° C. under N₂. The resulting mixture was stirred at 20° C. for 16 hr. After completion, the reaction mixture was diluted with CH₂Cl₂ (800 mL) and washed with H₂O (500 mL*3), brine (500 mL), dried with anhydrous Na₂SO₄, filtered and concentrated in vacuum to give a crude product. The crude product was triturated with (PE/EtOAc=10/1, 500 mL*2) at 25° C. for 1 hr to give compound 23-8 (230.6 g, 648.76 mmol, 93.3% yield) as a light yellow solid.

¹H NMR (400 MHz, CDCl₃) δ=7.82 (d, J=8.0 Hz, 2H), 7.36 (d, J=8.0 Hz, 2H), 4.72-4.66 (m, 1H), 3.64-3.57 (m, 2H), 3.32-3.24 (m, 2H), 2.47 (s, 3H), 1.82-1.75 (m, 2H), 1.74-1.68 (m, 2H), 1.45 (s, 9H).

### Step 2: tert-butyl 4-(tritylthio)piperidine-1-carboxylate (23-9)

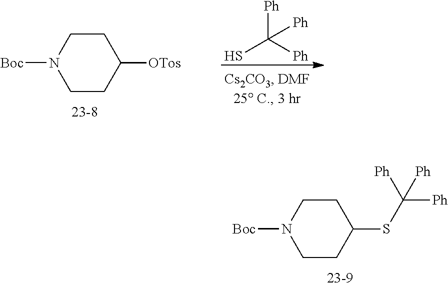

A mixture of tert-butyl 4-(p-tolylsulfonyloxy)piperidine-1-carboxylate (115 g, 323.54 mmol, 1 eq), triphenylmethanethiol (107.31 g, 388.24 mmol, 1.2 eq), NaI (2.42 g, 16.18 mmol, 0.05 eq), Cs₂CO₃ (158.12 g, 485.30 mmol, 1.5 eq) in DMF (700 mL) was degassed and purged with N₂ 3 times, and then the mixture was stirred at 50° C. for 3 hr under N₂ atmosphere. After completion, the reaction mixture was quenched by H₂O (1000 mL) and then diluted with EtOAc (800 mL). The aqueous phase was extracted with EtOAc (800 mL*3). The combined organic phase was washed with brine (600 mL*3), dried with anhydrous Na₂SO₄, filtered and concentrated in vacuum to give a residue. The residue was purified by column chromatography (SiO₂, PE/EtOAc=20/1 to 5/1) to give compound 23-9 (178.8 g, 389.00 mmol, 66.7% yield) as a yellow oil.

¹H NMR (400 MHz, CDCl₃) δ=7.43-7.40 (m, 6H), 7.21-7.17 (m, 6H), 7.13-7.11 (m, 3H), 3.62-3.60 (m, 2H), 2.60-2.53 (m, 2H), 2.42-2.31 (m, 1H), 2.26-2.14 (m, 1H), 2.10-2.01 (m, 1H), 1.48-1.43 (m, 2H), 1.32 (s, 9H).

### Step 3: 1-methyl-4-(tritylthio)piperidine (23-10)

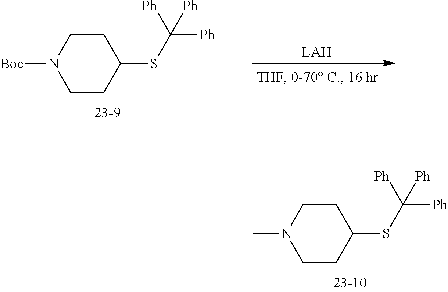 [SMILES: CC(C)(C)OC(=O)N1CCC(SC(c2ccccc2)(c2ccccc2)c2ccccc2)CC1 ; CN1CCC(SC(c2ccccc2)(c2ccccc2)c2ccccc2)CC1]

To a solution of tert-butyl 4-tritylsulfanylpiperidine-1-carboxylate (75 g, 163.17 mmol, 1 eq) in THF (1000 mL) was added LAH (9.29 g, 244.76 mmol, 1.5 eq) in portions at 0° C. under N₂. After addition, the mixture was stirred at 70° C. for 16 hr. After completion, the reaction mixture was diluted with THF (500 mL), then successively was added H₂O (9.3 mL), aq.NaOH (9.3 mL, 4M), H₂O (28 mL) and Na₂SO₄ (100 g) at 0° C. under N₂. The reaction mixture was filtered and the filtrate was concentrated in vacuum to give a residue. The residue was purified by flash silica gel chromatography (330 g SepaFlash® Silica Flash Column, MeOH/CH₂Cl₂: 0˜5%, 1% NH₃ in MeOH) to give compound 23-10 (47.8 g, 120.28 mmol, 44.10% yield, 94% purity) as a yellow oil.

¹H NMR (400 MHz, CDCl₃) δ=7.43-7.41 (m, 6H), 7.21-7.17 (m, 6H), 7.13-7.09 (m, 3H), 2.49-2.45 (m, 2H), 2.12-2.07 (m, 1H), 2.05 (s, 3H), 1.76-1.71 (m, 2H), 1.41-1.33 (m, 4H).

### Step 4: 1-methylpiperidine-4-thiol (23-3)

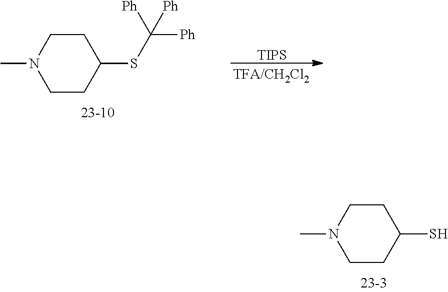

To a solution of 1-methyl-4-tritylsulfanyl-piperidine (7 g, 18.74 mmol, 1 eq) in CH₂Cl₂ (60 mL) were added TFA (30.80 g, 270.13 mmol, 20 mL, 14.42 eq) and TIPS (7.34 g, 37.48 mmol, 2 eq) at 0° C. under N₂. After addition, the resulting mixture was stirred at 20° C. for 16 hr. After completion, the reaction mixture was concentrated under reduced pressure to remove TFA and filtered. The filtrate was diluted with MeOH (100 mL) and extracted with PE (50 mL*5). The MeOH layers was concentrated under reduced pressure to give compound 23-3 (4.5 g, crude, TFA) as a yellow oil. The crude product was used in the next step without further purification.

¹H NMR (400 MHz, CDCl₃) δ=3.74-3.71 (m, 2H), 3.51-3.48 (m, 1H), 3.33-3.27 (m, 1H), 2.89-2.85 (m, 3H), 2.01-2.76 (m, 1H), 2.51-2.39 (m, 1H), 2.28-2.25 (m, 1H), 2.08-1.96 (m, 1H), 1.91-1.87 (m, 1H).

### Step 5: tert-butyl 4-((4-oxo-4-(pentadecan-8-yloxy)butyl)amino)butanoate (23-2)

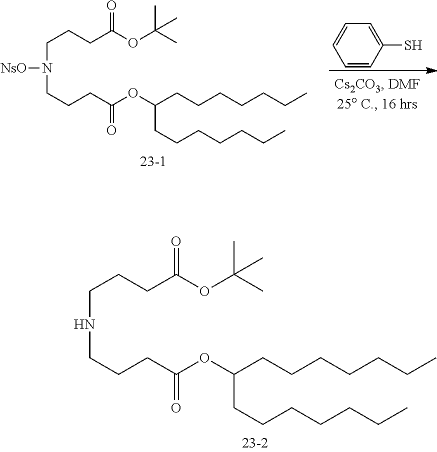

To a solution of 1-heptyloctyl 4-[(4-tert-butoxy-4-oxo-butyl)-(4-nitrophenyl)sulfonyl-amino]butanoate (15.6 g, 24.34 mmol, 1 eq) in DMF (100 mL) were added Cs₂CO₃ (15.86 g, 48.68 mmol, 2 eq) and benzenethiol (6.18 g, 56.09 mmol, 5.72 mL, 2.30 eq). The mixture was stirred at 25° C. for 16 hr under N₂. After completion, the reaction mixture was quenched by the addition of a solution of NaOH (150 mL, 1M), and then extracted with EtOAc (150 mL*3). The combined organic layers were washed with brine (60 mL*3), dried over sodium sulfate, filtered and concentrated under reduced pressure. The residue was purified by flash silica gel chromatography (40 g SepaFlash® Silica Flash Column, EtOAc: PE: 0˜35%) to give compound 23-2 (8.7 g, 19.09 mmol, 78.4% yield) as a yellow oil.

¹H NMR (400 MHz, CDCl₃) δ=4.90-4.84 (m, 1H), 2.64-2.61 (m, 4H), 2.33 (t, J=7.6 Hz, 2H), 2.25 (t, J=7.6 Hz, 2H), 1.80-1.76 (m, 4H), 1.53-1.48 (m, 4H), 1.45 (s, 9H), 1.30-1.26 (m, 20H), 0.90-0.86 (m, 6H).

### Step 6: tert-butyl 4-((((I-methylpiperidin-4-yl)thio)carbonyl)(4-oxo-4-(pentadecan-8-yloxy)butyl)amino)butanoate (23-4)

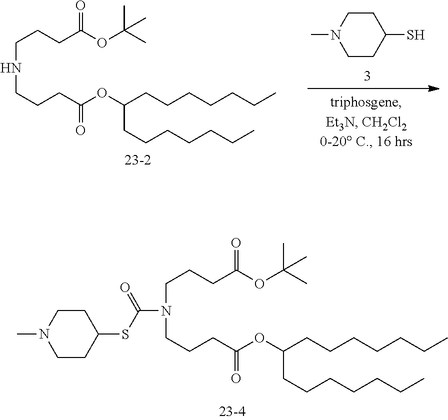

To a solution of 1-heptyloctyl 4-[(4-tert-butoxy-4-oxo-butyl)amino]butanoate (2 g, 4.39 mmol, 1 eq) dissolved in dry CH₂Cl₂ (30 mL) were added Et₃N (1.33 g, 13.17 mmol, 1.8 mL, 3 eq) and triphosgene (781.41 mg, 2.63 mmol, 0.6 eq) at 0° C. under N₂. The resulting solution was stirred at 20° C. for 1 hr. The resulting reaction was concentrated under reduced pressure and kept under N₂. To a solution of 1-methylpiperidine-4-thiol (3.77 g, 15.36 mmol, 3.5 eq, TFA) dissolved in dry THF (40 mL) was added NaOH (1.23 g, 30.72 mmol, 7 eq) at 0° C. under N₂. To this resulting solution, carbamoyl chloride, dissolved in THF (20 mL) was added via syringe slowly under N₂ at 0° C. The resulting solution was stirred at 20° C. for 15 hr. After completion, the reaction mixture was quenched by NH₄Cl (60 mL) at 0° C. and then diluted with EtOAc (50 mL). The aqueous phase was extracted with EtOAc (60 mL*3). The combined organic phase was washed with brine (50 mL), dried with anhydrous Na₂SO₄, filtered and concentrated in vacuum to give a residue. The residue was purified by flash silica gel chromatography (40 g SepaFlash® Silica Flash Column, EtOAc: PE: 0˜25%) to give compound 23-4 (1.5 g, 1.81 mmol, 42.7% yield, 74% purity) as a yellow oil.

### LCMS [M+H]⁺: 613.3

### Step 7: 4-((((1-methylpiperidin-4-yl)thio)carbonyl)(4-oxo-4-(tetradecan-7-yloxy)butyl)amino)butanoic acid (23-5)

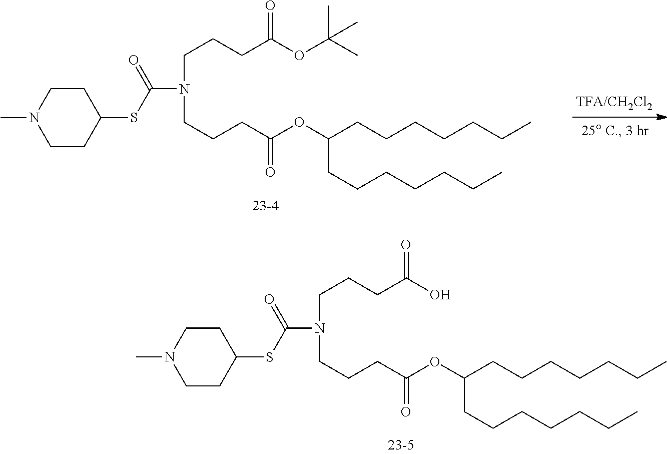

To a solution of 1-heptyloctyl 4-[(4-tert-butoxy-4-oxo-butyl)-[(1-methyl-4-piperidyl)sulfanylcarbonyl]amino]butanoate (1.5 g, 2.45 mmol, 1 eq) in CH₂Cl₂ (15 mL) was added TFA (7.70 g, 67.53 mmol, 5 mL) under N₂. The mixture was stirred at 25° C. for 3 hr. After completion, the reaction mixture was concentrated under reduced pressure to remove solvent to give compound 23-5 (1.6 g, crude, TFA) as a yellow oil. The crude product was used directly in the next step without further purification.

### Step 8: (Z)-non-2-en-1-yl 4-((((I-methylpiperidin-4-yl)thio)carbonyl)(4-oxo-4-(pentadecan-8-yloxy)butyl)amino)butanoate (CAT23)

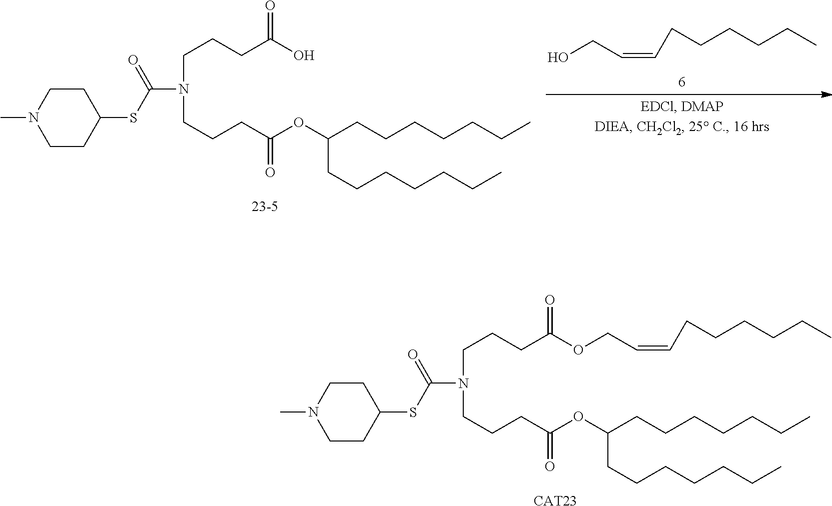

To a solution of 4-[[4-(1-heptyloctoxy)-4-oxo-butyl]-[(1-methyl-4-piperidyl)sulfanylcarbonyl]amino]butanoic acid (1.4 g, 2.09 mmol, 1 eq, TFA) in CH₂Cl₂ (20 mL) were added EDCI (1.20 g, 6.26 mmol, 3 eq), HOBt (845.9 mg, 6.26 mmol, 3 eq) and DIPEA (809.1 mg, 6.26 mmol, 1.1 mL, 3 eq) at 0° C. under N₂. After addition, the mixture was stirred at this temperature for 0.5 hr, and then (Z)-non-2-en-1-ol (890.5 mg, 6.26 mmol, 3 eq) was added dropwise. The resulting mixture was stirred at 20° C. for 15.5 hr. After completion, the reaction mixture was quenched by H₂O (60 mL) and then diluted with EtOAc (50 mL). The aqueous phase was extracted with EtOAc (50 mL*3). The combined organic phase was washed with brine (60 mL), dried with anhydrous Na₂SO₄, filtered and concentrated in vacuum to give a residue. The residue was purified by positive prep-HPLC (column: Welch Ultimate XB-CN 250*50*10 um; mobile phase: [Hexane-EtOH]; B %: 0%-15%, 8 min) to give CAT23 (682 mg, 0.98 mmol, 47.7% yield, 98% purity) as a light yellow oil.

### LCMS [M+H]⁺: 682.3

¹H NMR (400 MHz, CDCl₃) δ=5.66-5.61 (m, 1H), 5.55-5.50 (m, 1H), 4.88-4.85 (m, 1H), 4.63 (br d, J=6.4 Hz, 2H), 3.45-3.32 (m, 5H), 2.78-2.72 (m, 2H), 2.33-2.30 (m, 4H), 2.26 (s, 3H), 2.16-2.08 (m, 4H), 2.03-1.99 (m, 2H), 1.92-1.85 (m, 4H), 1.73-1.65 (m, 2H), 1.55-1.48 (m, 4H), 1.37-1.34 (m, 2H), 1.30-1.22 (m, 26H), 0.89-0.86 (m, 9H).

### Example 1.24: Synthesis of CAT24

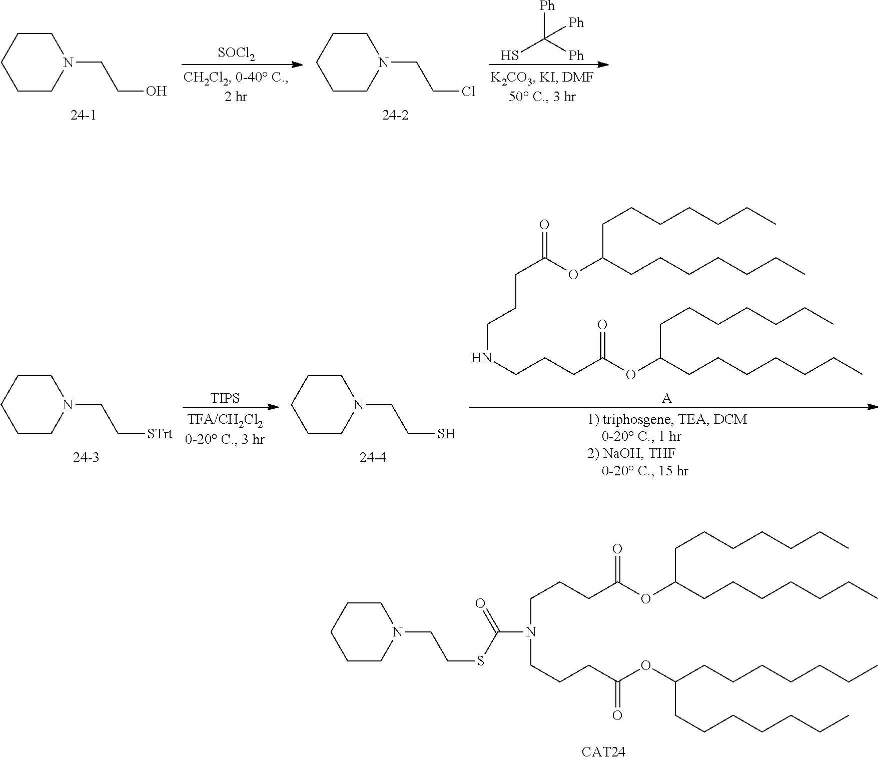

### Step 1: 1-(2-chloroethyl)piperidine (2): (EC2098-19)

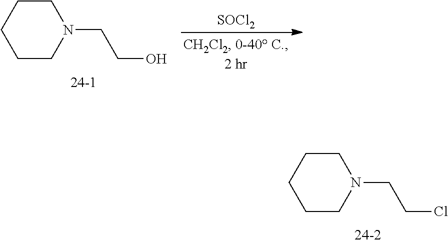 [SMILES: OCCN1CCCCC1 ; "SOCl2" ; ClCCN1CCCCC1]

To a solution of 2-(1-piperidyl)ethanol (5.00 g, 38.7 mmol, 5.14 mL, 1 eq) in dichloromethane (50.0 mL) was added SOCl₂ (13.8 g, 116 mmol, 8.42 mL, 3.00 eq), dropwise, slowly at 0° C. Then the mixture was stirred at 40° C. for 2 hours. The reaction mixture was concentrated under reduced pressure to give compound 24-2 (7.17 g, crude, HCl salt) as a white solid.

¹H NMR (400 MHz, DMSO-d₆) δ=11.0 (s, 1H), 4.06 (t, J=6.8 Hz, 2H), 3.42-3.40 (m, 4H), 2.95-2.89 (m, 2H), 1.86-1.78 (m, 4H), 1.70-1.67 (m, 1H), 1.41-1.31 (m, 1H).

### Step 2: 1-(2-(tritylthio)ethyl)piperidine (24-3)

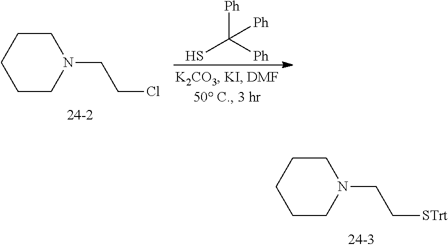

A mixture of 1-(2-chloroethyl)piperidine (5.00 g, 33.9 mmol, 1.00 eq), triphenylmethanethiol (11.2 g, 40.6 mmol, 1.20 eq), potassium carbonate (18.7 g, 135 mmol, 4.00 eq), potassium iodide (562 mg, 3.39 mmol, 0.10 eq) in DMF (50.0 mL) was degassed and purged with N₂ 3 times, and then the mixture was stirred at 50° C. for 3 hours under N₂ atmosphere. The reaction mixture was partitioned between ethyl acetate (100 mL) and water (100 mL). The organic phase was separated, washed with brine (60.0 mL×3), dried over anhydrous sodium sulfate, filtered and concentrated under reduced pressure to give a residue. The residue was purified by flash silica gel chromatography (ISCO®; 80 g SepaFlash® Silica Flash Column, Eluent of 0˜10% Ethyl acetate/Petroleum ethergradient @ 100 mL/min) to give compound 24-3 (5.70 g, 13.7 mmol, 40.6% yield, 93.4% purity) and (2.20 g, 4.92 mmol, 14.5% yield, 86.7% purity) as a white solid.

### LCMS [M+1]⁺: 388.2

¹H NMR (400 MHz, CDCl₃-d) δ=7.44-7.42 (m, 6H), 7.31-7.27 (m, 6H), 7.25-7.20 (m, 3H), 2.39-2.34 (m, 2H), 2.31-2.26 (m, 2H), 2.22 (s, 4H), 1.54-1.49 (m, 4H), 1.40-1.37 (m, 2H).

### Step 3: 2-(piperidin-1-yl)ethanethiol (24-4)

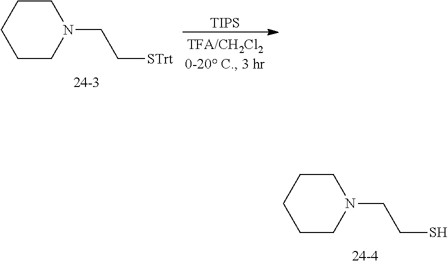

A mixture of 1-(2-tritylsulfanylethyl)piperidine (6.50 g, 16.8 mmol, 1.00 eq) in TFA (20.0 mL) and dichloromethane (60.0 mL), the mixture was degassed and purged with N₂ 3 times, then triisopropylsilane (5.31 g, 33.5 mmol, 6.89 mL, 2 eq) was added slowly at 0° C., and then the mixture was stirred at 20° C. for 3 hours under N₂ atmosphere. The reaction mixture was concentrated under reduced pressure to remove TFA and filtered. The filtrate was diluted with methanol (150 mL) and extracted with petroleum ether (50.0 mL×5). The methanol layers were concentrated under reduced pressure to give a crude product to give compound 24-4 (4.30 g, 16.6 mmol, 98.9% yield, TFA salt) as a light yellow oil.

¹H NMR (400 MHz, DMSO-d₆) δ=3.45-3.42 (m, 2H), 3.19-3.12 (m, 2H), 2.89-2.80 (m, 5H), 1.80-1.77 (m, 2H), 1.66-1.63 (m, 3H), 1.38-1.35 (m, 1H).

### Step 4: 2-(piperidin-1-yl)ethanethiol (CAT24)

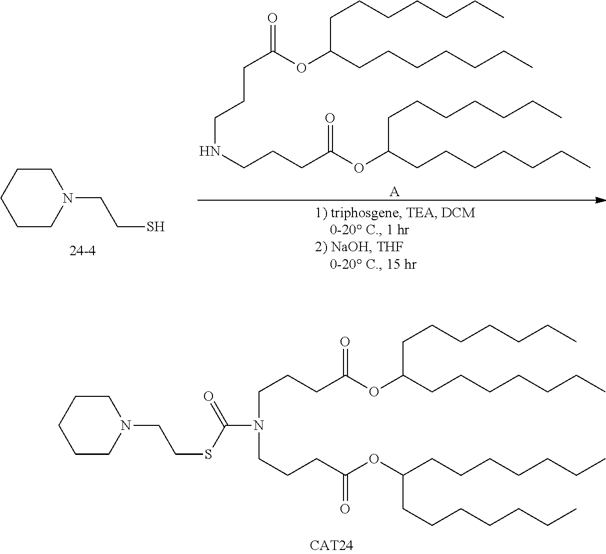

To a solution of 1-heptyloctyl 4-[[4-(1-heptyloctoxy)-4-oxo-butyl]amino]butanoate (2.00 g, 3.28 mmol, 1.00 eq) dissolved in dry dichloromethane (20.0 mL) were added TEA (995 mg, 9.84 mmol, 1.37 mL, 3.00 eq) and triphosgene (876 mg, 2.95 mmol, 0.90 eq) at 0° C. under N₂. The resulting solution was stirred at 20° C. for 1 hour. The resulting reaction was concentrated under reduced pressure. To a 2-(1-piperidyl)ethanethiol (2.98 g, 11.5 mmol, 3.50 eq, TFA salt) dissolved in dry THF (25.0 mL) was added NaOH (1.97 g, 49.2 mmol, 15.0 eq) at 0° C. under nitrogen atmosphere,. To this resulting solution, carbamoyl chloride, dissolved in THF (20.0 mL), was added via syringe slowly under N₂ at 0° C. The resulting solution was stirred at 20° C. for 15 hours. The reaction mixture was quenched by ammonium chloride (20.0 mL) at 0° C. and then diluted with ethyl acetate (60.0 mL). The aqueous phase was extracted with ethyl acetate (50.0 mL×3). The combined organic phase was washed with brine (60.0 mL), dried with anhydrous sodium sulfate, filtered and concentrated in vacuum to give a residue. The residue was purified by flash silica gel chromatography (ISCO®; 120 g SepaFlash® Silica Flash Column, Eluent of 0˜50% Ethyl acetate/Petroleum ethergradient @100 mL/min) to give compound CAT24 (1.09 g, 1.38 mmol, 43.3% yield, 99.2% purity) as a light yellow oil.

### LCMS [M+1]⁺: 782.4

¹H NMR (400 MHz, CDCl₃-d) δ=4.90-4.84 (m, 2H), 3.38 (s, 4H), 3.05-3.01 (m, 2H), 2.57-2.53 (m, 2H), 2.46 (s, 4H), 2.31 (s, 4H), 1.90 (s, 4H), 1.61-1.56 (m, 4H), 1.52-1.51 (m, 8H), 1.46-1.43 (m, 2H), 1.32-1.27 (m, 40H), 0.90-0.87 (m, 12H).

### Example 1.25: Synthesis of CAT25

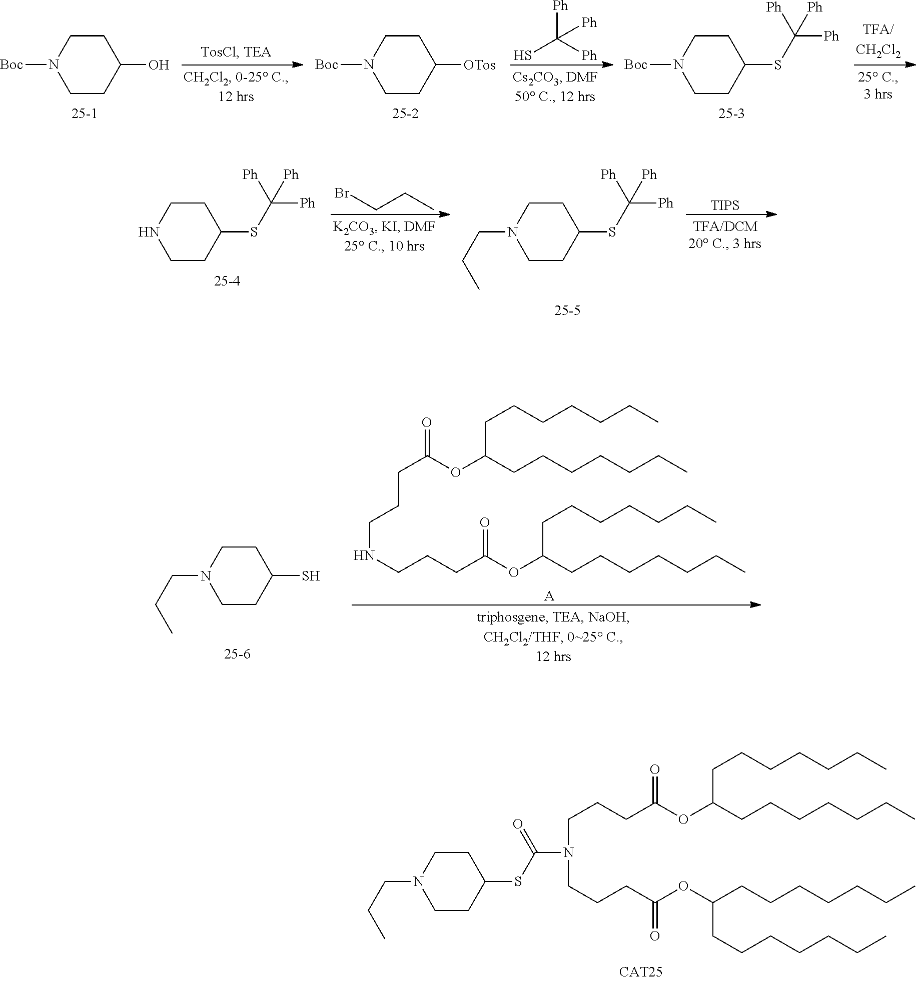

### Step 1: tert-butyl 4-(tosyloxy)piperidine-1-carboxylate (25-2)

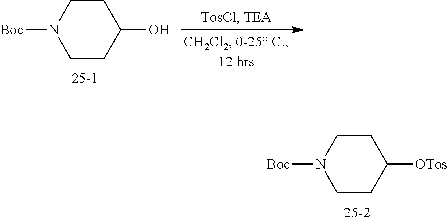

To a solution of tert-butyl 4-hydroxypiperidine-1-carboxylate (50.0 g, 248 mmol, 1 eq.) in CH₂Cl₂ (1000 mL) were added TEA (50.3 g, 497 mmol, 69.2 mL, 2 eq.), DMAP (1.52 g, 12.4 mmol, 0.05 eq.) and 4-methylbenzenesulfonyl chloride (71.0 g, 373 mmol, 1.5 eq.) under N₂ at 0° C. The mixture was stirred at 25° C. for 12 hours. The reaction mixture was diluted with CH₂Cl₂ (500 mL), and extracted with water (500 mL×3) and brine (500 mL), dried with anhydrous Na₂SO₄, filtered and concentrated under reduced pressure to give a residue. The crude product was triturated with petroleum ether: ethyl acetate (10: 1, 500 mL) at 25° C. for 10 min to give compound 25-2 (320 g, 900 mmol, 91% yield) as a white solid.

¹H NMR (400 MHz, CDCl₃) δ=7.79 (d, J=8.4 Hz, 2H), 7.34 (d, J=8.0 Hz, 2H), 4.75-4.60 (m, 1H), 3.65-3.52 (m, 2H), 3.32-3.19 (m, 2H), 2.45 (s, 3H), 1.83-1.72 (m, 2H), 1.71-1.62 (m, 2H), 1.43 (s, 9H)

### Step 2: tert-butyl 4-(tritylthio)piperidine-1-carboxylate (25-3)

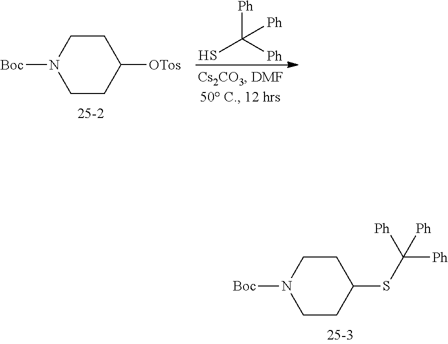

A mixture of tert-butyl 4-(tosyloxy)piperidine-1-carboxylate (160 g, 450 mmol, 1 eq.), triphenylmethanethiol (149 g, 540 mmol, 1.2 eq.), NaI (3.37 g, 22.5 mmol, 0.05 eq.), Cs₂CO₃ (219 g, 675 mmol, 1.5 eq.) in DMF (1600 mL) was degassed and purged with N₂ 3 times, and then the mixture was stirred at 50° C. for 12 hours under N₂ atmosphere. The reaction mixture was filtered, and the filtrate was extracted with ethyl acetate (1000 mL×3) and water (1000 mL), The combined organic layers were washed with brine (1000 mL×3), dried over Na₂SO₄, filtered and concentrated under reduced pressure to give compound 25-3 (410 g, crude) as a yellow solid.

### Step 3: 4-(tritylthio)piperidine (25-4)

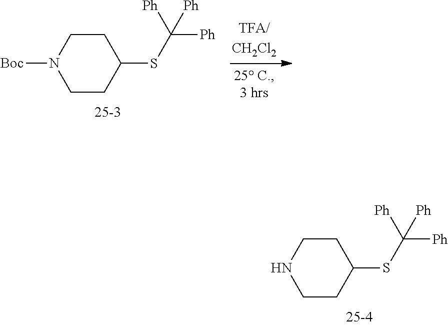 [SMILES: CC(C)(C)OC(=O)N1CCC(SC(c2ccccc2)(c2ccccc2)c2ccccc2)CC1 ; c1ccc(C(SC2CCNCC2)(c2ccccc2)c2ccccc2)cc1]

To a solution of tert-butyl 4-(tritylthio)piperidine-1-carboxylate (100 g, 218 mmol, 1 eq.) in CH₂Cl₂ (1000 mL) was added TFA (308 g, 2.70 mol, 200 mL, 12.4 eq.). The mixture was stirred at 25° C. for 3 hours. The reaction was washed and concentrated with CH₂Cl₂ (500 mL) for 4 times. The residue was triturated with MTBE at 25° C. for 1 hour to give compound 25-4 (75.0 g, 121 mmol, 48.2% yield, 57.8% purity) as a white solid.

¹H NMR (400 MHz, CDCl₃) δ=9.06-8.93 (m, 1H), 7.45-7.34 (m, 6H), 7.26-7.17 (m, 7H), 7.16-7.09 (m, 2H), 3.05 (s, 2H), 2.63 (s, 2H), 2.51-2.39 (m, 1H), 2.38-2.28 (m, 1H), 1.71-1.61 (m, 1H), 1.49-1.33 (m, 2H).

### Step 4: 1-propyl-4-(tritylthio)piperidine (25-5)

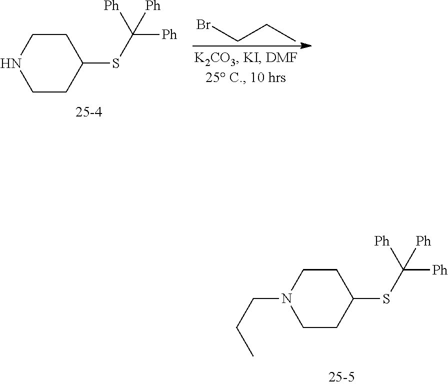

To a solution of 4-(tritylthio)piperidine (15 g, 41.7 mmol, 1 eq.) and 1-bromopropane (4.62 g, 37.6 mmol, 3.42 mL, 0.9 eq.) in DMF (150 mL) were added K₂CO₃ (28.83 g, 209 mmol, 5 eq.) and KI (693 mg, 4.17 mmol, 0.1 eq.). The mixture was stirred at 25° C. for 10 hours. The reaction mixture was quenched by the addition of 300 mL at 25° C., and extracted with ethyl acetate (100 mL×3). The combined organic layers were washed with brine, dried over Na₂SO₄, filtered and concentrated under reduced pressure to give a residue. The residue was purified by flash silica gel chromatography (ISCO®; 330 g SepaFlash® Silica Flash Column, Eluent of 0˜30% Ethyl acetate/Petroleum ether gradient @ 100 mL/min). The residue was purified by MPLC(Column I.D. 100 mm*H300 mm Welch Ultimate XB_C18 20-40 μm; 120 A; Flow rate 200 ml/min; Mobile phase H₂O+ACN; Gradient B % 10-45% 20 min; 45% 5 min) to give compound 25-5(6.58 g, 12.5 mmol, 29.9% yield, 98% purity, TFA) as a white solid.

### LCMS [M+1]⁺: 402.3

¹H NMR (400 MHz, CDCl₃) δ=12.69-11.85 (m, 1H), 7.60-7.35 (m, 6H), 7.27-7.18 (m, 6H), 7.16-7.03 (m, 3H), 3.41-3.13 (m, 2H), 2.88-2.60 (m, 4H), 2.24-1.82 (m, 3H), 1.77-1.53 (m, 2H), 1.42-1.14 (m, 2H), 0.94-0.74 (m, 3H).

### Step 5: 1-propylpiperidine-4-thiol (25-6)

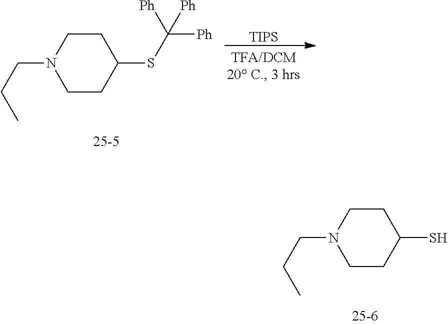

To a solution of 1-propyl-4-(tritylthio)piperidine (6.50 g, 16.2 mmol, 1 eq.) in TFA (20.0 mL) and CH₂Cl₂ (60.0 mL) was added triisopropylsilane (5.13 g, 32.4 mmol, 6.65 mL, 2 eq.). The mixture was stirred at 25° C. for 3 hours. The reaction mixture was concentrated under reduced pressure to give a residue. The residue was dissolved in methanol (10.0 mL), and extrated with petroleum ether (10.0 mL×5), The combined methanol layers was concentrated under reduced pressure to give 1-propylpiperidine-4-thiol (4.42 g, crude, TFA) as a yellow oil.

¹H NMR (400 MHz, DMSO-d₆) δ=3.50-3.13 (m, 3H), 3.10-2.83 (m, 5H), 2.20-2.03 (m, 2H), 1.86-1.55 (m, 4H), 0.95-0.85 (m, 3H).

### Step 6: di(pentadecan-8-yl) 4,4′-((((1-propylpiperidin-4-yl)thio)carbonyl)azanediyl)dibutanoate (CAT25)

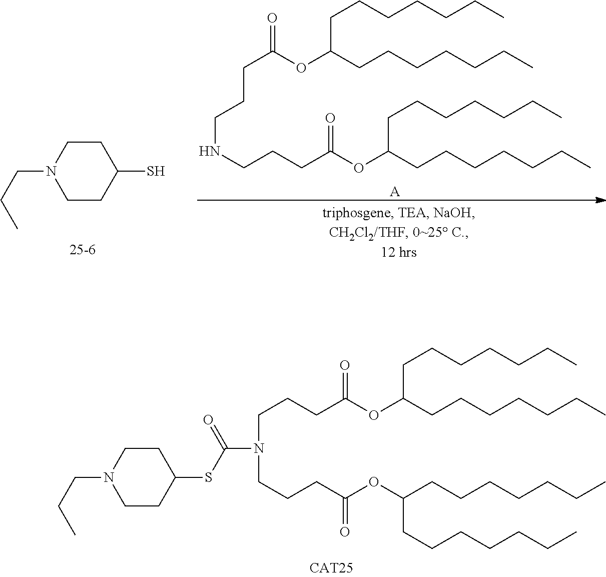

To a solution of di(pentadecan-8-yl) 4,4′-azanediyldibutanoate (2.80 g, 4.59 mmol, 1 eq.) dissolved in dry CH₂Cl₂ (40.0 mL) were added TEA (1.39 g, 13.8 mmol, 1.92 mL, 3 eq.) and triphosgene (1.24 g, 4.18 mmol, 0.91 eq.) at 0° C. under N₂. The resulting solution was stirred at 20° C. for 1 hour. To a 1-propylpiperidine-4-thiol (4.39 g, 16.1 mmol, 3.50 eq., TFA) dissolved in dry THF (40.0 mL) at 0° C. under nitrogen atmosphere, was added NaOH (1.84 g, 45.9 mmol, 10.0 eq.) under nitrogen atmosphere. To this resulting solution, carbamoyl chloride, dissolved in THF (10.0 mL), was added via syringe slowly under N₂ at 0° C. The resulting solution was stirred at 20° C. for 11 hours. The reaction mixture was quenched by NH₄Cl (50.0 mL) at 0° C. and then diluted with ethyl acetate (50.0 mL). The aqueous phase was extracted with ethyl acetate (50.0 mL×3). The combined organic phase was washed with brine (30.0 mL), dried with anhydrous Na₂SO₄, filtered and concentrated in vacuum to give a residue. The residue was purified by flash silica gel chromatography (ISCO®; 220 g SepaFlash® Silica Flash Column, Eluent of 0˜35% Ethyl acetate/Petroleum ether gradient @100 mL/min) to give CAT25 (0.85 g, 1.06 mmol, 23.1% yield, 99.1% purity) as a yellow oil.

### LCMS [M+1]⁺: 796.4

¹H NMR (400 MHz, CDCl₃) δ=4.90-4.80 (m, 2H), 3.37 (s, 5H), 2.83 (d, J=9.2, 2H), 2.40-2.23 (m, 6H), 2.21-2.08 (m, 2H), 2.06-1.97 (m, 2H), 1.90 (s, 4H), 1.76-1.67 (m, 2H), 1.52 (s, 1OH), 1.27 (s, 40H), 0.98-0.76 (m, 15H).

### Example 1.26: Synthesis of CAT26

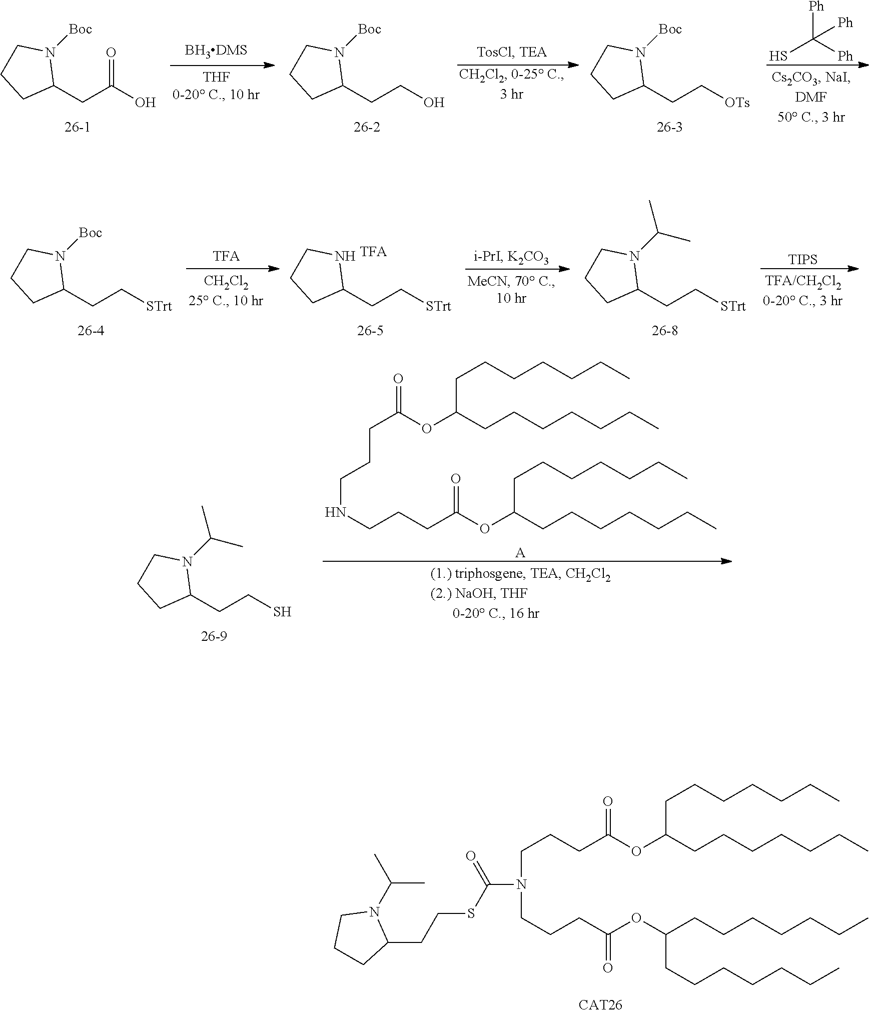

### Step 1: tert-butyl 2-(2-hydroxyethyl)pyrrolidine-1-carboxylate (26-2)

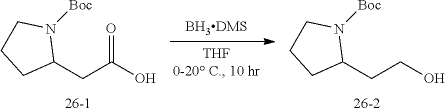 [SMILES: CC(C)(C)OC(=O)N1CCCC1CC(=O)O ; CC(C)(C)OC(=O)N1CCCC1CCO]

To a solution of 2-(1-tert-butoxycarbonylpyrrolidin-2-yl)acetic acid (50.0 g, 218 mmol, 1.00 eq) in THF (600 mL) was added BH₃-Me₂S (10.0 M, 32.7 mL, 1.50 eq) at 0° C. via Syringe dropwise over 30 min under a nitrogen atmosphere, then the mixture was stirred at 20° C. for 9.5 h under nitrogen atmosphere. The reaction was quenched by meihanol (100 mL) and concentrated, then the residue was diluted with ethyl acetate (300 mL) and H₂O (350 mL), extracted with ethyl acetate (200 mL×3), washed by brine (500 mL), dried over anhydrous sodium sulfate, filtered and concentrated under reduced pressure to give a residue. The aqueous phase quenched by sodium hypochlorite solution and discarded. The residue was purified by column chromatography (SiO₂, Petroleum ether/Ethyl acetate=50/1 to 3/1) to give compound 2 (30.0 g, 132 mmol, 60.7% yield, 95.0% purity) as a colorless oil.

### LCMS [M+23]⁺: 238.1

¹H NMR (400 MHz, DMSO-d₆) δ=4.37 (t, J=5.2 Hz, 1H), 3.73 (s, 1H), 3.42-3.38 (m, 2H), 3.23-3.19 (m, 2H), 1.83-1.66 (m, 6H), 1.39 (s, 9H).

### Step 2: tert-butyl 2-[2-(p-tolylsulfonyloxy)ethyl]pyrrolidine-1-carboxylate (26-3)

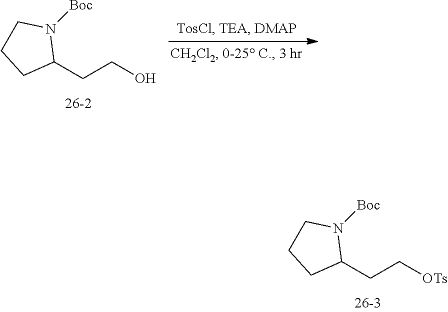 [SMILES: CC(C)(C)OC(=O)N1CCCC1CCO ; "CH2Cl2, 0-25° C., 3 hr" ; Cc1ccc(S(=O)(=O)OCCC2CCCN2C(=O)OC(C)(C)C)cc1]

A mixture of tert-butyl 2-(2-hydroxyethyl)pyrrolidine-1-carboxylate (27.0 g, G12-273, C₃ mmol, 1.00 eq), TEA (25.4 g, 251 mmol, 34.9 mL, 2.00 eq) and DMAP (766 mg, 6.27 mmol, 0.05 eq) in dichloromethane (450 mL) was degassed and purged with N₂₃ times, then TosCl (35.9 g, 188 mmol, 1.50 eq) was added slowly at 0° C., and then the mixture was stirred at 25° C. for 3 hours under N₂ atmosphere. The residue was diluted with dichloromethane (200 mL), the combined organic layers were washed with H₂O (450 mL) and brine (450 mL), dried over anhydrous sodium sulfate, filtered and concentrated under reduced pressure to give a residue. The residue was purified by column chromatography (SiO₂, Petroleum ether/Ethyl acetate=50/1 to 3/1)to give compound 26-3 (23.2 g, 49.5 mmol, 39.5% yield, 78.9% purity) as a yellow oil.

### LCMS [M-100+1]⁺: 270.1

¹H NMR (400 MHz, MeOD-d₄) δ=7.80 (d, J=2.0 Hz, 1H) 7.81-7.79 (m, 1H), 7.72 (s, 1H), 7.46 (s, 1H), 7.25 (s, 1H), 4.09-4.03 (m, 2H), 3.79-3.77 (m, 1H), 3.49-3.43 (m, 2H), 2.37 (s, 3H), 2.00-1.95 (m, 2H), 1.66-1.49 (m, 4H), 1.42 (s, 9H).

### Step 3: tert-butyl 2-(2-tritylsulfanylethyl)pyrrolidine-1-carboxylate (26-4)

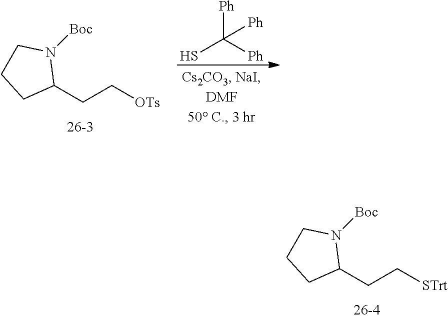

A mixture of tert-butyl 2-[2-(p-tolylsulfonyloxy)ethyl]pyrrolidine-1-carboxylate (23.0 g, 62.3 mmol, 1 eq), triphenylmethanethiol (20.7 g, 74.7 mmol, 1.20 eq), Cs₂CO₃ (30.4 g, 93.4 mmol, 1.5 eq), NaI (933 mg, 6.23 mmol, 0.10 eq) in DMF (200 mL) was degassed and purged with N₂ 3 times, and then the mixture was stirred at 50° C. for 3 hours under N₂ atmosphere. The reaction mixture was partitioned between ethyl acetate (1500 mL) and H₂O (1000 mL). The organic phase was separated, washed with brine (300 mL×2), dried over anhydrous sodium sulfate, filtered and concentrated under reduced pressure to give a residue. The residue was purified by column chromatography (SiO₂, Petroleum ether/Ethyl acetate=1/0 to 5/1) to give compound 26-4 (20.6 g, 39.3 mmol, 63.2% yield, 90.6% purity) as a yellow oil.

¹H NMR (400 MHz, CDCl₃-d) δ=7.46-7.41 (m, 6H), 7.33-7.27 (m, 6H), 7.25-7.20 (m, 3H), 3.68 (s, 1H), 3.31 (s, 1H), 3.20 (s, 1H), 2.15 (s, 2H), 1.76-1.65 (m, 4H), 1.43 (s, 9H), 1.39-1.34 (m, 2H)

### Step 4: 2-(2-tritylsulfanylethyl)pyrrolidine (26-5)

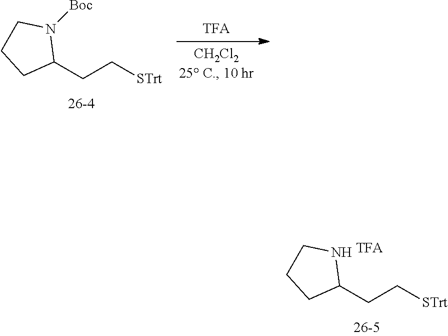

To a solution of tert-butyl 2-(2-tritylsulfanylethyl)pyrrolidine-1-carboxylate (20.6 g, 43.4 mmol, 1 eq) in dichloromethane (200 mL) was added TFA (61.6 g, 540 mmol, 40.0 mL, 12.5 eq). The mixture was stirred at 25° C. for 10 hours. The reaction mixture was concentrated under reduced pressure to remove dichloromethane and TFA. The residue was purified by prep-MPLC (MeCN: H₂O: 0˜45%) to give compound 26-5 (16.2 g, 32.2 mmol, 74.3% yield, 97.0% purity, TFA salt) as a yellow solid.

### LCMS [M+1]⁺: 374.1

¹H NMR (400 MHz, CDCl₃-d) δ=7.32-7.30 (m, 6H), 7.21-7.17 (m, 6H), 7.14-7.13 (m, 3H), 3.31 (s, 1H), 3.11 (s, 2H), 2.20-2.12 (m, 2H), 1.84-1.79 (m, 3H), 1.50-1.43 (m, 1H), 1.33-1.31 (m, 1H), 1.20-1.16 (m, 1H).

### Step 5: 1-isopropyl-2-(2-tritylsulfanylethyl)pyrrolidine (26-6)

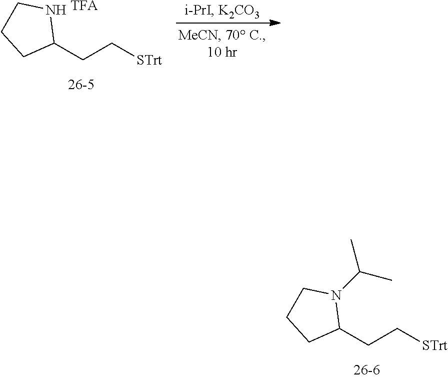

To a solution of 2-(2-tritylsulfanylethyl)pyrrolidine (8.00 g, 16.4 mmol, 1.00 eq, TFA) and 2-iodopropane (3.07 g, 18.1 mmol, 1.80 mL, 1.10 eq) in MeCN (80.0 mL) was added K₂CO₃ (6.80 g, 49.2 mmol, 3.00 eq). The mixture was stirred at 70° C. for 10 hours. The reaction mixture was filtered and concentrated under reduced pressure to give a residue. The residue was purified by flash silica gel chromatography (ISCO®; 220 g SepaFlash® Silica Flash Column, Eluent of 0˜10% Methanol/Dichloromethanegradient @ 100 mL/min) to give compound 26-6 (4.60 g, 11.1 mmol, 67.7% yield) as a brown red solid.

### LCMS [M+1]⁺: 416.5

¹H NMR (400 MHz, CDCl₃-d) δ=7.40-7.37 (m, 6H), 7.27-7.22 (m, 6H), 7.20-7.16 (m, 3H), 3.01-2.92 (m, 2H), 2.79-2.75 (m, 1H), 2.53-2.47 (m, 1H), 2.29-2.23 (m, 1H), 2.12-2.05 (m, 1H), 1.75-1.61 (m, 4H), 1.56-1.49 (m, 1H), 1.38-1.30 (m, 1H), 1.14 (d, J=6.4 Hz, 3H), 0.97 (d, J=6.4 Hz, 3H)

### Step 6: 2-(I-isopropylpyrrolidin-2-yl)ethanethiol (26-9)

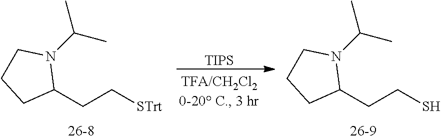

A mixture of 1-isopropyl-2-(2-tritylsulfanylethyl)pyrrolidine (4.10 g, 9.86 mmol, 1.00 eq) in TFA (14.0 mL) and dichloromethane (42.0 mL), the mixture was degassed and purged with N₂ 3 times, then triisopropylsilane (3.12 g, 19.7 mmol, 4.05 mL, 2.00 eq) was added slowly at 0° C., and then the mixture was stirred at 20° C. for 3 hours under N₂ atmosphere. The reaction mixture was concentrated under reduced pressure to remove TFA and filtered. The filtrate was diluted with methanol (70.0 mL) and extracted with petroleum ether (50.0 mL×5). The methanol layers was concentrated under reduced pressure to give compound 26-9 (2.69 g, crude, TFA) as a yellow oil.

¹H NMR (400 MHz, CDCl₃-d) δ=3.76-3.69 (m, 3H), 3.09-3.00 (m, 1H), 2.92-2.84 (m, 1H), 2.46-2.41 (m, 1H), 2.27-2.12 (m, 5H), 2.04-1.88 (m, 2H), 1.47 (d, J=6.4 Hz, 3H), 1.36 (d, J=6.8 Hz, 3H)

### Step 7: 1-heptyloctyl 4-[[4-(I-heptyloctoxy)-4-oxo-butyl]-[2-(I-isopropylpyrrolidin-2-yl)ethylsulfanylcarbonyl]amino]butanoate (ONC-SM-027-NX-1): (ECI092-33 34)

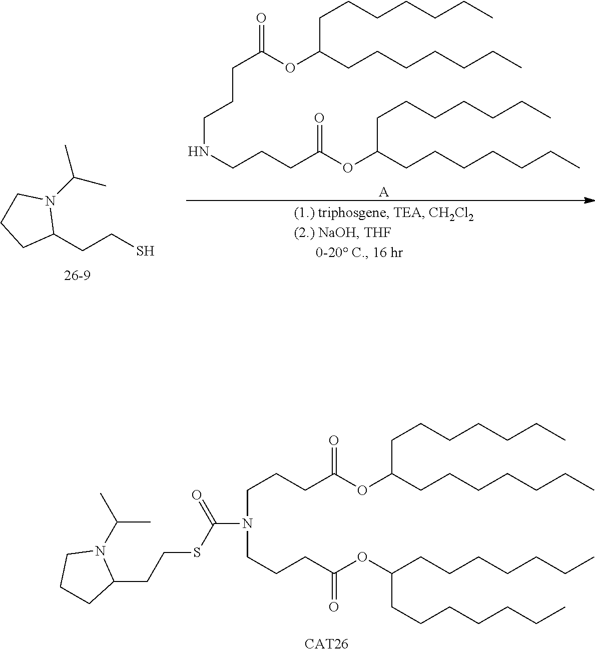

To a solution of 1-heptyloctyl 4-[[4-(1-heptyloctoxy)-4-oxo-butyl]amino]butanoate (1.90 g, 3.11 mmol, 1.00 eq) dissolved in dry dichloromethane (20.0 mL) were added TEA (946 mg, 9.34 mmol, 1.30 mL, 3.00 eq) and triphosgene (760 mg, 2.56 mmol, 0.82 eq) at 0° C. under N₂. The resulting solution was stirred at 20° C. for 1 hour. The resulting reaction was concentrated under reduced pressure. To 2-(1-isopropylpyrrolidin-2-yl)ethanethiol (2.68 g, 9.34 mmol, 3.00 eq, TFA) dissolved in dry THF (25.0 mL) was added NaOH (1.87 g, 46.7 mmol, 15.0 eq) at 0° C. under nitrogen atmosphere. To this resulting solution, carbamoyl chloride, dissolved in THF, (20.0 mL) was added via syringe slowly under N₂ at 0° C. The resulting solution was stirred at 20° C. for 15 hours. The reaction mixture was quenched by NH₄Cl (50.0 mL) at 0° C. and then diluted with ethyl acetate (60.0 mL). The aqueous phase was extracted with ethyl acetate (60.0 mL×3). The combined organic phase was washed with brine (50.0 mL), dried with anhydrous sodium sulfate, filtered and concentrated in vacuum to give a residue. The residue was purified by flash silica gel chromatography (ISCO®; 120 g SepaFlash® Silica Flash Column, Eluent of 0˜50% Ethyl acetate/Petroleum ethergradient @100 mL/min) to give compound CAT26 (610 mg, 0.742 mmol, 21.3% yield, 98.4% purity) as a yellow oil.

### LCMS [M+1]⁺: 810.6

¹H NMR (400 MHz, CDCl₃-d) δ=4.90-4.84 (m, 2H), 3.38 (s, 4H), 2.99-2.92 (m, 2H), 2.90-2.80 (m, 2H), 2.79-2.75 (m, 1H), 2.52-2.46 (m, 1H), 2.31 (s, 4H), 1.89 (d, J=4.8 Hz, 6H), 1.79-1.67 (m, 4H), 1.52 (d, J=5.2 Hz, 8H), 1.27 (s, 40H), 1.12 (d, J=6.8 Hz, 3H), 0.97 (d, J=6.4 Hz, 3H), 0.88 (t, J=6.8 Hz, 12H).

### Example 1.27: Synthesis of CAT27

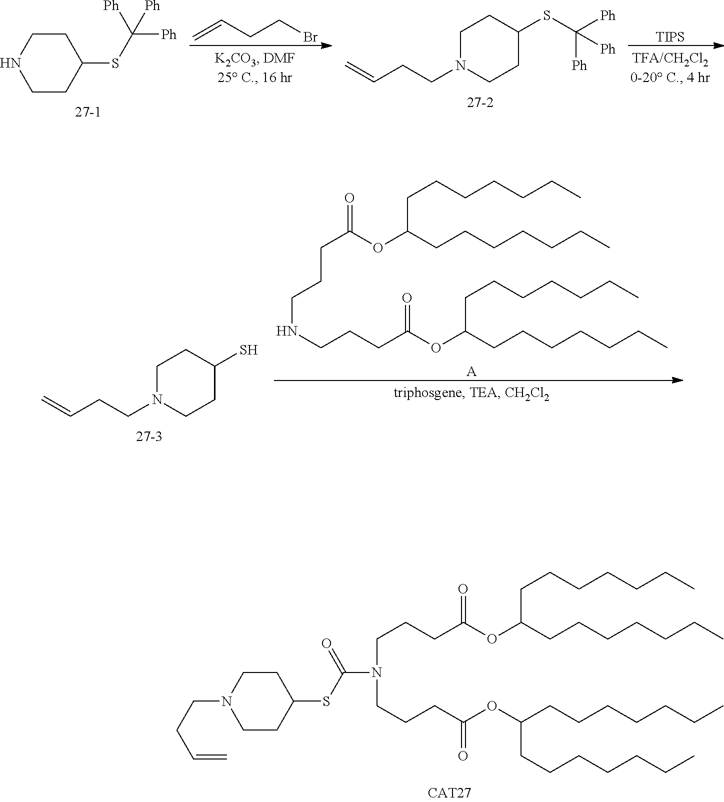

### Step 1: 1-(but-3-en-1-yl)-4-(tritylthio)piperidine (27-2)

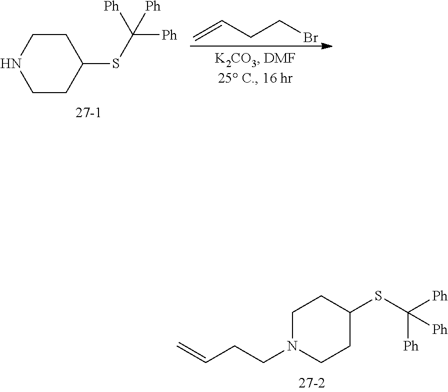

To a solution of 4-tritylsulfanylpiperidine (20 g, 42.23 mmol, 1 eq, TFA) in DMF (120 mL) were added K₂CO₃ (17.51 g, 126.70 mmol, 3 eq) and 4-bromobut-1-ene (5.13 g, 38.01 mmol, 3.86 mL, 0.9 eq). The mixture was stirred at 25° C. for 16 hr. After completion, the reaction mixture was quenched by H₂O (150 mL) and then diluted with EtOAc (100 mL). The aqueous phase was extracted with EtOAc (150 mL*3). The combined organic phase was washed with brine (100 mL*3), dried with anhydrous Na₂SO₄, filtered and concentrated in vacuum to give a residue. The residue was purified by flash silica gel chromatography (80 g SepaFlash® Silica Flash Column, EtOAc: PE: 0˜20%, 1% NH₃·H₂O in EtOAc) and inverted MPLC (MeCN: H₂O: 0˜45%) to give compound 27-2 (9.6 g, 22.51 mmol, 65.6% yield, 97% purity) as a yellow solid.

### LCMS [M+H]⁺: 414.6

¹H NMR (400 MHz, CDCl₃) δ=12.27-12.21 (m, 1H), 7.42-7.36 (m, 6H), 7.23-7.18 (m, 6H), 7.16-7.10 (m, 3H), 5.64-5.55 (m, 1H), 5.09-5.00 (m, 2H), 3.40-3.25 (m, 3H), 2.87-2.79 (m, 4H), 2.42-2.36 (m, 2H), 2.21-2.16 (m, 1H), 2.07-1.99 (m, 2H), 1.25-1.18 (m, 1H).

### Step 2: 1-(but-3-en-1-yl)piperidine-4-thiol (27-3)

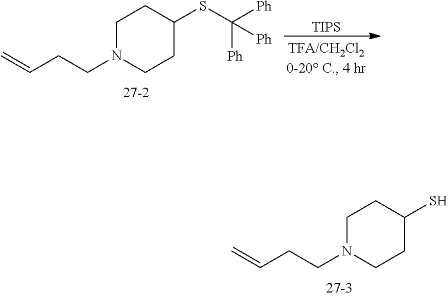 [SMILES: C=CCCN1CCC(SC(c2ccccc2)(c2ccccc2)c2ccccc2)CC1 ; C=CCCN1CCC(S)CC1 ; "TIPS"]

To a solution of 1-but-3-enyl-4-tritylsulfanyl-piperidine (9.5 g, 22.97 mmol, 1 eq) in CH₂Cl₂ (80 mL) were added TFA (36.58 g, 320.78 mmol, 23.8 mL, 13.97 eq) and TIPS (9.00 g, 45.94 mmol, 2 eq) at 0° C. under N₂. After addition, the resulting mixture was stirred at 20° C. for 4 hr. After completion, the reaction mixture was concentrated under reduced pressure to remove TFA and filtered. The filtrate was diluted with MeOH (150 mL) and extracted with PE (50 mL*5). The MeOH layers was concentrated under reduced pressure to give compound 27-3 (6.4 g, crude, TFA) as a yellow oil. The crude product was used in the next step without further purification.

### Step 6: di(pentadecan-8-yl) 4,4′-((((1-(but-3-en-1-yl)piperidin-4-yl)thio)carbonyl)azanediyl)dibutanoate (CAT27)

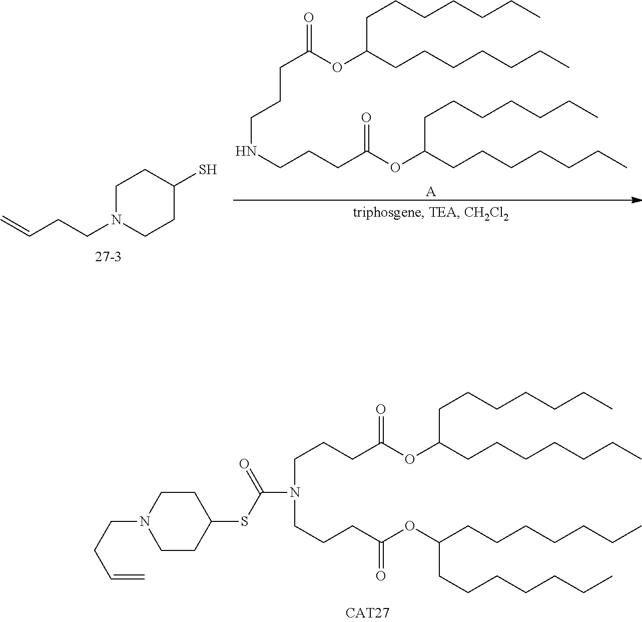

To a solution of 1-heptyloctyl 4-[[4-(1-heptyloctoxy)-4-oxo-butyl]amino]butanoate (4.5 g, 7.38 mmol, 1 eq) dissolved in dry CH₂Cl₂ (50 mL)were added TEA (2.24 g, 22.13 mmol, 3.1 mL, 3 eq) and triphosgene (1.31 g, 4.43 mmol, 0.6 eq) at 0° C. under N₂. The resulting solution was stirred at 20° C. for 1 hr. The resulting reaction was concentrated under reduced pressure. To a solution of 1-but-3-enylpiperidine-4-thiol (6.31 g, 22.13 mmol, 3 eq, TFA) dissolved in dry THF (35 mL) was added NaOH (2.07 g, 51.64 mmol, 7 eq) at 0° C. under N₂. To this resulting solution, carbamoyl chloride, dissolved in THF (30 mL), was added via syringe slowly under N₂ at 0° C. The resulting solution was stirred at 20° C. for 15 hr. After completion, the reaction mixture was quenched by NH₄Cl (100 mL) at 0° C. and then diluted with EtOAc (100 mL). The aqueous phase was extracted with EtOAc (100 mL*3). The combined organic phase was washed with brine (120 mL), dried with anhydrous Na₂SO₄, filtered and concentrated in vacuum to give a residue. The residue was purified by flash silica gel chromatography (40 g SepaFlash® Silica Flash Column, EtOAc: PE: 0˜40%) and positive prep-HPLC (column: Welch Ultimate XB-CN 250*50*10 um; mobile phase: [Neu-ETOH];B %: 0%-10%, 8 min) to afford CAT27 (488 mg, 0.59 mmol, 59.9% yield, 98.2% purity) as a light yellow oil.

### LCMS [M+H]⁺: 808.3

¹H NMR (400 MHz, CD30D-d₄) δ=5.86-5.79 (m, 1H), 5.13-5.01 (m, 2H), 4.93-4.89 (m, 2H), 3.48-3.38 (m, 5H), 2.89-2.86 (m, 2H), 2.48-2.43 (m, 2H), 2.35-2.23 (m, 8H), 2.08-2.03 (m, 2H), 1.93-1.88 (m, 4H), 1.76-1.67 (m, 2H), 1.61-1.56 (m, 8H), 1.35-1.25 (m, 40H), 0.94-0.90 (m, 12H).

### Example 1.28: Synthesis of CAT28

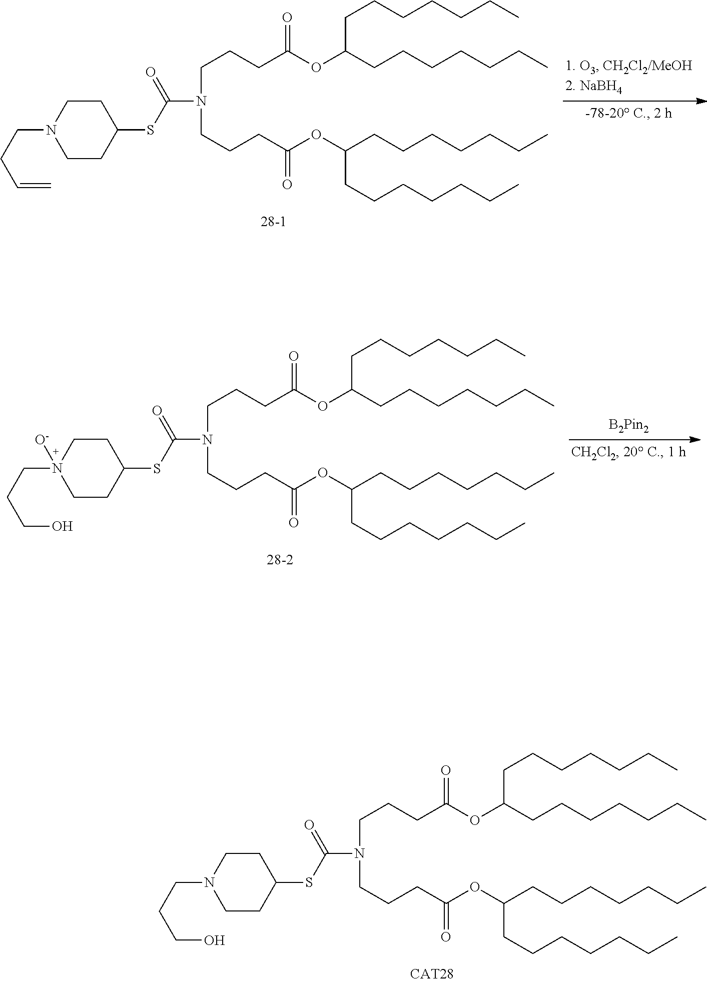

### Step 1: 4-((bis(4-oxo-4-(pentadecan-8-yloxy)butyl)carbamoyl)thio)-1-(3-hydroxypropyl)piperidine 1-oxide (28-2)

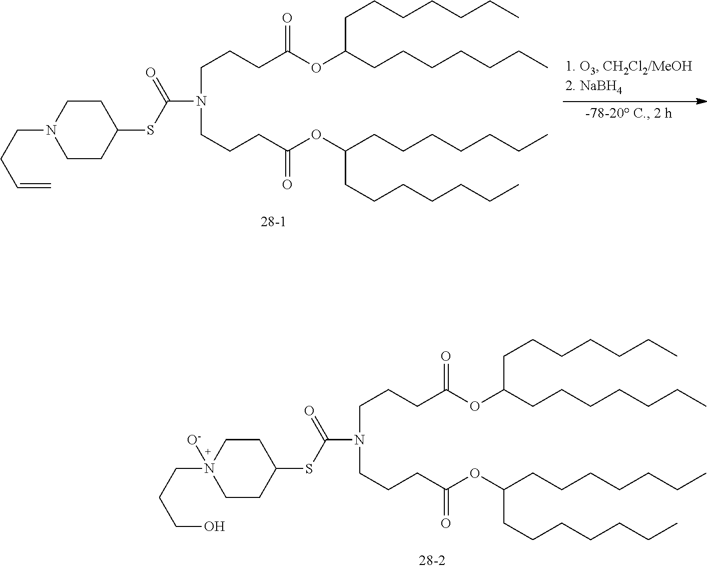

To a solution of 1-heptyloctyl 4-[(1-but-3-enyl-4-piperidyl)sulfanylcarbonyl-[4-(1-heptyloctoxy)-4-oxo-butyl]amino]butanoate (1.2 g, 1.49 mmol, 1 eq) in CH₂Cl₂ (20 mL) and MeOH (10 mL) was cooled to −78° C., and a stream of Ozone (71.35 mg, 1.49 mmol, 1 eq) (15 Psi) was bubbled into the reaction mixture until a light blue color became evident. 02 was then bubbled through the reaction mixture until the blue color disappeared and then was added NaBH₄ (112.47 mg, 2.97 mmol, 2 eq). The reaction mixture was stirred at 20° C. for 2 hr. After completion, the reaction mixture gave compound 28-2 (1.23 g, crude) as a yellow liquid. The reaction mixture was used directly in the next step without further purification.

Step 2: di(pentadecan-8-yl) 4,4′-((((1-(3-hydroxypropyl)piperidin-4-yl)thio)carbonyl)azanediyl)dibutanoate (CAT28)

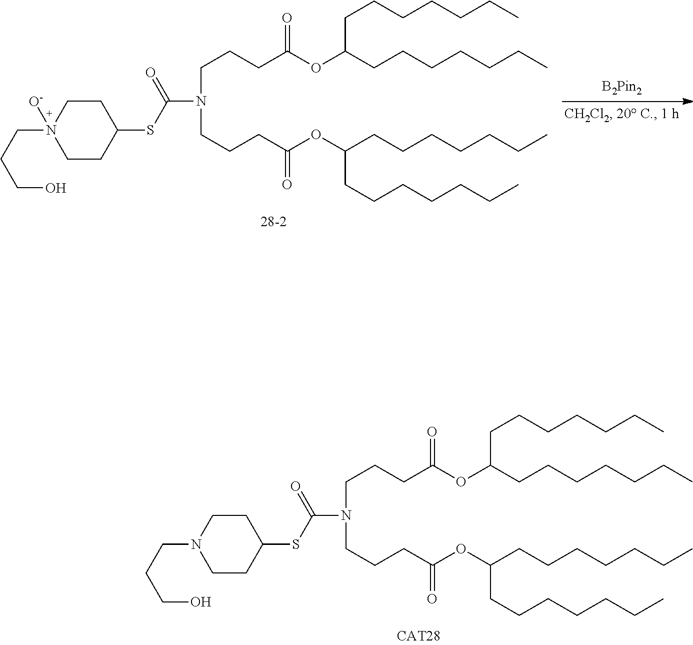

To a solution of 1-heptyloctyl 4-[[4-(1-heptyloctoxy)-4-oxo-butyl]-[1-(3-hydroxypropyl)-1-oxido-piperidin-1-ium-4-yl]sulfanylcarbonyl-amino]butanoate (1.23 g, 1.49 mmol, 1 eq) in CH₂Cl₂ (10 mL) was added BPD (755.11 mg, 2.97 mmol, 2 eq). The mixture was stirred at 25° C. for 1 hr. After completion, the reaction mixture was quenched by H₂O (60 mL) and then diluted with EtOAc (50 mL). The aqueous phase was extracted with EtOAc (50 mL*3). The combined organic phase was washed with brine (60 mL), dried with anhydrous Na₂SO₄, filtered and concentrated in vacuum to give a residue. The residue was purified by flash silica gel chromatography (40 g SepaFlash® Silica Flash Column, Ethyl acetate: Petroleum ether: 0˜20%) and positive prep-HPLC (column: Welch Ultimate XB-CN 250*50 *10 um; mobile phase: [Hexane-EtOH]; B %: 0%-30%, 10 min) to afford CAT28 (248 mg, 296.82 umol, 20.07% yield, 97.1% purity) as a yellow oil.

### LCMS [M+H]⁺: 811.6

¹H NMR (400 MHz, CDCl₃) δ=4.90-4.84 m, 2H), 3.80 (t, J=5.2 Hz, 2H), 3.38-3.34 (m, 5H), 2.95-2.90 (m, 2H), 2.60 (t, J=5.6 Hz, 2H), 2.33-2.27 (m, 4H), 2.23-2.16 (m, 2H), 2.08-2.01 (m, 2H), 1.93-1.85 (m, 4H), 1.74-1.62 (m, 4H), 1.55-1.48 (br s, 8H), 1.33-1.25 (m, 40H), 0.91-0.86 (m, 12H).

### Example 1.29: Synthesis of CAT29

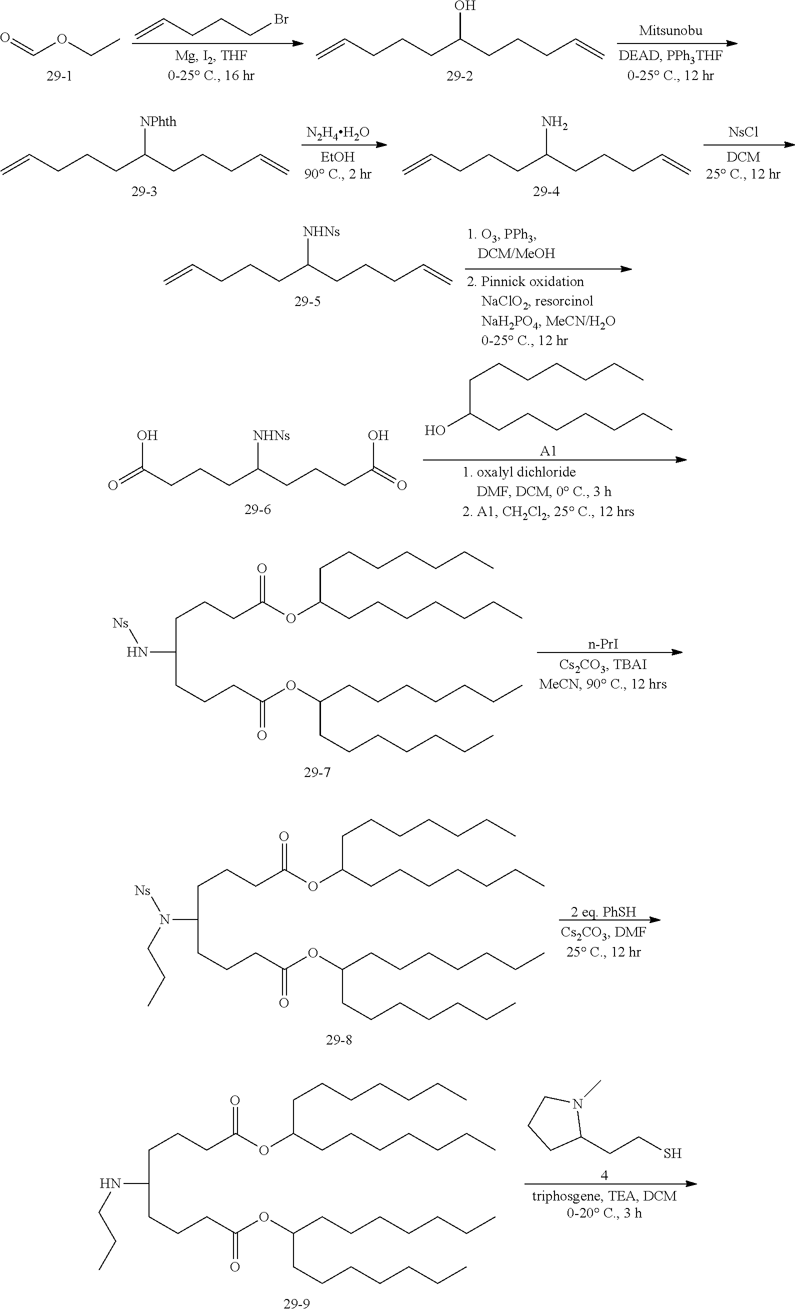

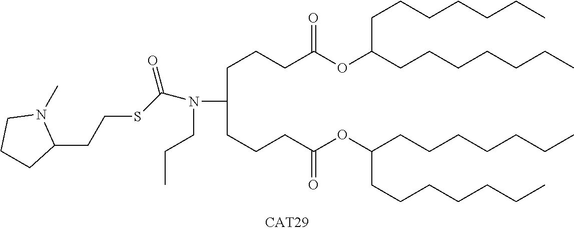 [SMILES: CCCCCCCC(CCCCCCC)OC(=O)CCCC(CCCC(=O)OC(CCCCCCC)CCCCCCC)N(CCC)C(=O)SCCC1CCCN1C]

### Step 1: undeca-1,10-dien-6-ol (29-2)

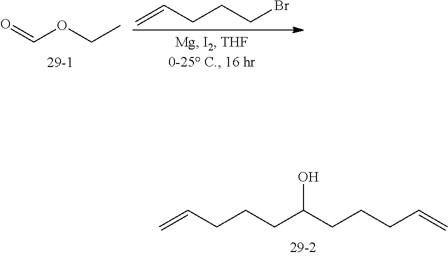

A suspension of I₂ (3.43 g, 13.50 mmol, 2.72 mL, 0.02 eq) and Mg (41.83 g, 1.72 mol, 2.55 eq) in dry THF (1500 mL) was prepared under nitrogen atmosphere. To this mixture, 5-bromopent-1-ene (251.47 g, 1.69 mol, 2.5 eq) was added slowly at 25° C. During the addition, an increase in the temperature of the reaction mixture confirmed the initiation of the Grignard formation. Once the addition of the bromide was completed, the mixture was stirred at 25° C. for 1 hr, after which it was cooled down to 0° C. for the slow addition of ethyl formate (50 g, 674.96 mmol, 54.29 mL, 1 eq). After the addition, the cold bath was removed and the mixture was stirred at 25° C. for 15 hr. The reaction was cooled down to 0° C. for quenching by the addition of saturated solution NH₄Cl (1000 mL) and stirred for 30 minutes. The aqueous phase was extracted with EtOAc (1000 mL×3). The combined organic phase was washed with brine (400×2 mL), dried with anhydrous Na₂SO₄, filtered and concentrated in vacuum to give a crude product. The crude product was purified by silica gel chromatography (Petroleum ether/Ethyl acetate=30/1 to 5/1) to give compound 29-2 (105 g, 623.98 mmol, 92.45% yield) as a yellow oil.

¹H NMR (400 MHz, CDCl₃) δ=5.82-5.76 (m, 2H), 5.01-4.92 (m, 4H), 3.58-3.57 (m, 1H), 2.06-2.02 (m, 4H), 1.53-1.50 (m, 1H), 1.48-1.41 (m, 8H).

### Step 2: 2-(1-pent-4-enylhex-5-enyl)isoindoline-1,3-dione (29-3)

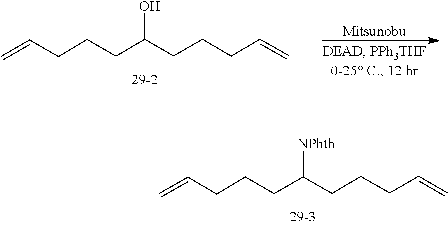

To a solution of undeca-1,10-dien-6-ol (66 g, 392.21 mmol, 1 eq) and isoindoline-1,3-dione (69.25 g, 470.66 mmol, 1.2 eq) in THF (800 mL) was added PPh3 (154.31 g, 588.32 mmol, 1.5 eq), then DIAD (237.93 g, 1.18 mol, 228.78 mL, 3 eq) was added, dropwise, at 0° C. The mixture was stirred at 25° C. for 12 hr. The reaction was quenched by the addition of saturated solution NH₄Cl (1000 mL) and the aqueous phase was extracted with EtOAc (1000 mL×3). The combined organic phase was washed with brine (500×2 mL), dried with anhydrous Na₂SO₄, filtered and concentrated in vacuum to give a crude product. The crude product was purified by silica gel chromatography (Petroleum ether/Ethyl acetate=30/1 to 5/1) to give compound 29-3 (100 g, 336.26 mmol, 85.73% yield) as a yellow oil.

¹H NMR (400 MHz, CDCl₃) δ=7.84-7.82 (m, 2H), 7.73-7.71 (m, 2H), 5.78-5.71 (m, 2H), 5.31-5.23 (m, 4H), 4.24-4.14 (m, 1H), 2.15-2.05 (m, 4H), 1.76-1.70 (m, 2H), 1.33-1.28 (m, 6H).

### Step 3: undeca-1,10-dien-6-amine (29-4)

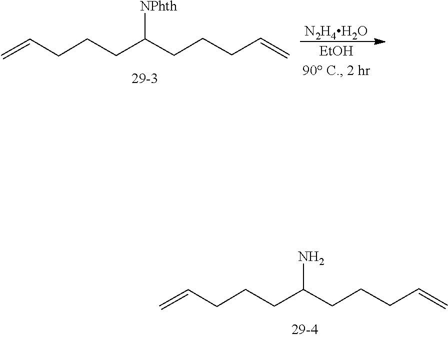

To a solution of 2-(1-pent-4-enylhex-5-enyl)isoindoline-1,3-dione (250 g, 840.65 mmol, 1 eq) in EtOH (1000 mL) was added N₂H₄·H₂O (85.88 g, 1.68 mol, 83.38 mL, 98% purity, 2 eq). The mixture was stirred at 95° C. for 2 hr. The reaction mixture was filtered three times and the filtrate was concentrated. The crude was dissolved in EtOAc (500 mL) and the organic phase was washed with water (500 mL×3), dried over Na₂SO₄, filtered and concentrated in vacuum to give compound 29-4 (130 g, 777.09 mmol, 92.44% yield) as a yellow oil.

¹H NMR (400 MHz, CDCl₃) δ=5.83-5.76 (m, 2H), 5.01-4.92 (m, 4H), 2.71-2.68 (m, 1H), 2.05-2.02 (m, 4H), 1.45-1.27 (m, 8H).

### Step 4: 4-nitro-N-(1-pent-4-enylhex-5-enyl)benzenesulfonamide (29-5)

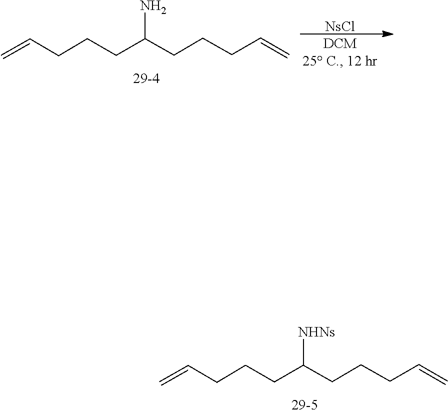

To a solution of undeca-1,10-dien-6-amine (60 g, 358.66 mmol, 1 eq) and 4-nitrobenzenesulfonyl chloride (87.43 g, 394.52 mmol, 1.1 eq) in CH₂Cl₂ (500 mL) was added TEA (72.58 g, 717.32 mmol, 99.84 mL, 2 eq). The mixture was stirred at 25° C. for 12 hr. The reaction mixture was quenched by the addition of water (500 mL) and then extracted with CH₂Cl₂ (1000 mL×3). The combined organic layers were washed with brine (500 mL), dried over sodium sulfate, filtered and concentrated under reduced pressure. The residue was purified by silica gel chromatography (Petroleum ether/Ethyl acetate=20/1 to 3/1) to give compound 29-5 (60 g, 170.24 mmol, 47.47% yield) as a yellow oil.

¹H NMR (400 MHz, CDCl₃) δ=8.37 (d, J=8.8 Hz, 2H), 8.08 (d, J=8.8 Hz, 2H), 5.71-5.62 (m, 2H), 4.93-4.89 (m, 4H), 3.35-3.30 (m, 1H), 1.97-1.91 (m, 4H), 1.33-1.25 (m, 8H).

### Step 5: 5-[(4-nitrophenyl)sulfonylamino]nonanedioic acid (29-6)

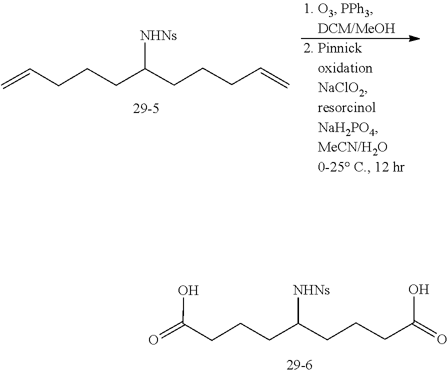

First, a solution of 4-nitro-N-(1-pent-4-enylhex-5-enyl)benzenesulfonamide (20 g, 56.75 mmol, 1 eq) in CH₂Cl₂ (200 mL) and MeOH (200 mL) was cooled to −70° C., and OZONE (136.19 mg, 2.84 mmol) was bubbled into the reaction mixture until a light blue color became evident. N₂ was then bubbled through the reaction mixture until the blue color disappeared. Then PPh3 (44.65 g, 170.24 mmol, 3 eq) was added, the reaction was stirred at 20° C. for 12 hr. The reaction mixture was concentrated in vacuum to give a residue. The residue was purified by silica gel chromatography (Petroleum ether/Ethyl acetate=20/1 to 1/1) to give compound 4-nitro-N-[5-oxo-1-(4-oxobutyl)pentyl]benzenesulfonamide (12.6 g, 35.35 mmol, 62.30% yield) as a yellow oil. Second, to a solution of 4-nitro-N-[5-oxo-1-(4-oxobutyl)pentyl]benzenesulfonamide (12 g, 33.67 mmol, 1 eq) in ACN (150 mL) were added benzene-1,3-diol (18.54 g, 168.35 mmol, 28.09 mL, 5 eq) and sodium; dihydrogen phosphate (1 M, 101.01 mL, 3 eq), then sodium chlorite (1 M, 168.35 mL, 5 eq) in water (150 mL) was added dropwise at 0° C. The mixture was stirred at 25° C. for 12 hr. The reaction mixture was neutralized to pH=2-3 with aq.HCl (4 M). The aqueous phase was extracted with EtOAc (500 mL×3). The combined organic phase successively was washed with saturated aqueous Na₂SO₃ (200 mL×3) and brine (100 mL×2), dried over Na₂SO₄, filtered and concentrated in vacuum to give a residue. The residue was purified by silica gel chromatography (Petroleum ether/Ethyl acetate=20/1 to 0/1) to give compound 29-6 (5.8 g, 14.93 mmol, 44.35% yield) as a yellow solid.

¹H NMR (400 MHz, DMSO-d₆) δ=11.94 (s, 2H), 8.39 (d, J=8.8 Hz, 2H), 8.04 (d, J=8.8 Hz, 2H), 7.95 (d, J=8.0 Hz, 1H), 3.15-3.14 (m, 1H), 2.03 (t, J=5.6 Hz, 4H), 1.33-1.23 (m, 8H).

### Step 6: bis(1-heptyloctyl) 5-[(4-nitrophenyl)sulfonylamino]nonanedioate (29-7)

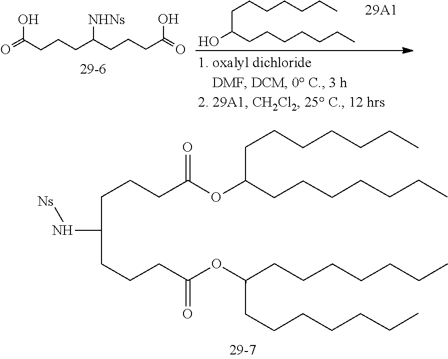

First, to a solution of 5-[(4-nitrophenyl)sulfonylamino]nonanedioic acid (2 g, 5.15 mmol, 1 eq) in CH₂Cl₂ (20 mL) were added oxalyl dichloride (1.96 g, 15.45 mmol, 1.35 mL, 3 eq) and DMF (3.76 mg, 51.49 umol, 3.96 uL, 0.01 eq). The mixture was stirred at 0° C. for 2 hr. The reaction mixture was concentrated under reduced pressure to give compound 5-[(4-nitrophenyl)sulfonylamino]nonanedioyl dichloride (2 g, 4.70 mmol, 91.33% yield) as a yellow oil. Second, to a solution of pentadecan-8-ol (2.15 g, 9.41 mmol, 2 eq) in CH₂Cl₂ (30 mL) was added 5-[(4-nitrophenyl)sulfonylamino]nonanedioyl dichloride (2 g, 4.70 mmol, 1 eq). The mixture was stirred at 25° C. for 12 hr. The reaction mixture was concentrated under reduced pressure to give a residue. The residue was purified by silica gel chromatography (Petroleum ether/Ethyl acetate=20/1 to 1/1) to give compound 29-7 (2.5 g, 3.09 mmol, 65.70% yield) as a yellow oil.

¹H NMR (400 MHz, CDCl₃) δ=8.36 (d, J=8.8 Hz, 2H), 8.08 (d, J=8.8 Hz, 2H), 4.87-4.82 (m, 2H), 3.31-3.30 (m, 1H), 2.21 (t, J=6.0 Hz, 4H), 1.50-1.41 (m, 16H), 1.32-1.26 (m, 40H), 0.90-0.87 (m, 12H).

### Step 7: bis(1-heptyloctyl) 5-[(4-nitrophenyl)sulfonyl-propyl-amino]nonanedioate (29-8)

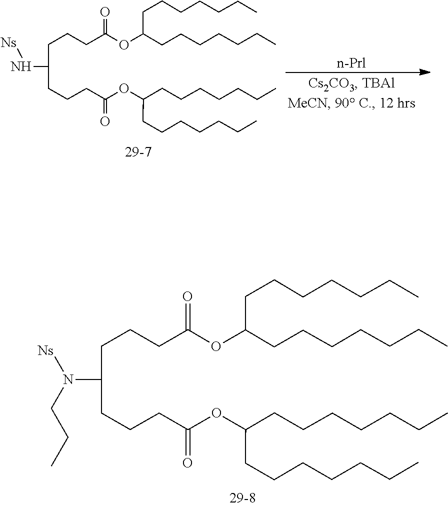

To a solution of bis(1-heptyloctyl) 5-[(4-nitrophenyl)sulfonylamino]nonanedioate (5 g, 6.18 mmol, 1 eq) and 1-iodopropane (3.15 g, 18.54 mmol, 1.81 mL, 3 eq) in DMF (80 mL) were added Cs₂CO₃ (6.04 g, 18.54 mmol, 3 eq), KI (512.86 mg, 3.09 mmol, 0.5 eq) and TBAI (1.14 g, 3.09 mmol, 0.5 eq). The mixture was stirred at 120° C. for 12 hr. The reaction mixture was quenched with saturated aqueous water (200 mL) and then diluted with EtOAC (200 mL). The aqueous phase was extracted with EtOAC (200 mL×3). The combined organic phase was washed with brine (300 mL), dried with anhydrous Na₂SO₄, filtered and concentrated in vacuum to give a residue. The residue was purified by silica gel chromatography (Petroleum ether/Ethyl acetate=40/1 to 5/1) to give compound 29-8 (5 g, 5.87 mmol, 95.06% yield) as a yellow oil.

### LCMS: [M+Na]+: 873.6;

### Step 8: bis(1-heptyloctyl) 5-(propylamino)nonanedioate (29-9)

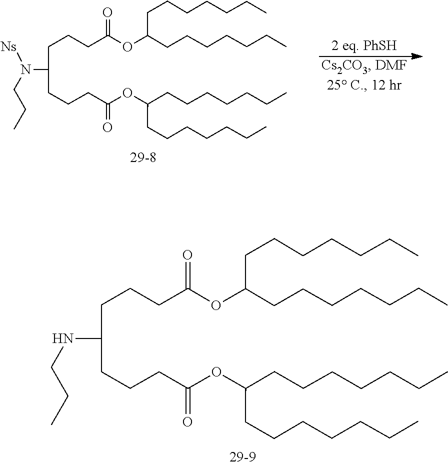

To a solution of bis(1-heptyloctyl) 5-[(4-nitrophenyl)sulfonyl-propyl-amino]nonanedioate (5 g, 5.87 mmol, 1 eq) in DMF (100 mL) was added Cs₂CO₃ (3.83 g, 11.74 mmol, 2 eq), then benzenethiol (1.86 g, 16.88 mmol, 1.72 mL, 2.88 eq) was added dropwise. The mixture was stirred at 25° C. for 12 hr under N₂. The reaction mixture was quenched by the addition of water (400 mL), and then extracted with EtOAC (500 mL×2). The combined organic layers were washed with brine (300 mL), dried over sodium sulfate, filtered and concentrated under reduced pressure. The residue was purified by silica gel chromatography (Petroleum ether/Ethyl acetate=10/1 to 0/1) to give compound 29-9 (1.7 g, 2.55 mmol, 43.48% yield) as a yellow oil.

¹H NMR (400 MHz, CDCl₃) δ=4.91-4.84 (m, 2H), 2.56-2.54 (m, 2H), 2.30 (t, J=7.6 Hz, 4H), 1.66-1.51 (m, 4H), 1.48-1.46 (m, 16H), 1.30-1.26 (m, 40H), 0.94-0.87 (m, 15H).

### Step 9: bis(1-heptyloctyl) 5-[2-(I-methylpyrrolidin-2-yl)ethylsulfanylcarbonyl-propyl-amino]nonanedioate (CAT29)

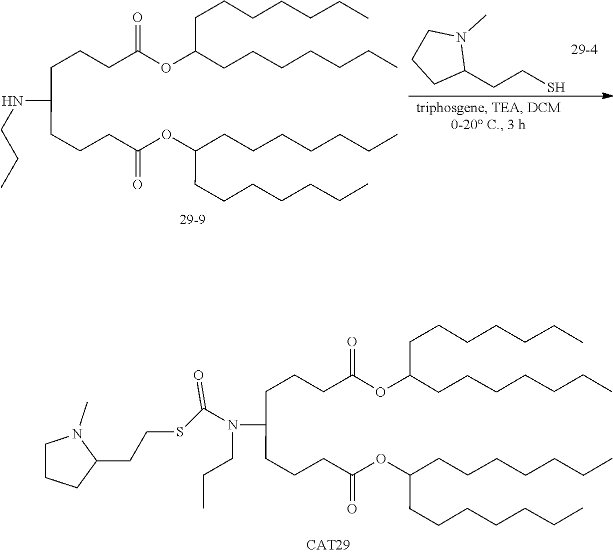

To a solution of bis(1-heptyloctyl) 5-(propylamino)nonanedioate (1.5 g, 2.25 mmol, 1 eq) in dry CH₂Cl₂ (20 mL) were added TEA (683.60 mg, 6.76 mmol, 940.30 uL, 3 eq) and bis(trichloromethyl) carbonate (334.12 mg, 1.13 mmol, 0.5 eq) at 0° C. under N₂ atmosphere. The resulting solution was stirred at 20° C. for 1 hr. The reaction was concentrated under reduced pressure and kept under N₂ atmosphere. NaOH (630.48 mg, 15.76 mmol, 7 eq) was dissolved in dry THF (50 mL) at 0° C., then 2-(1-methylpyrrolidin-2-yl)ethanethiol (1.64 g, 11.26 mmol, 5 eq) was added under N₂ atmosphere. To this resulting solution, carbamoyl chloride in THF (50 mL) was added slowly at 0° C. The mixture was stirred at 25° C. for 2 hr. The reaction mixture was quenched with saturated aqueous NH₄C₁ (100 mL) and then diluted with EtOAC (100 mL). The aqueous phase was extracted with EtOAC (100 mL×3). The combined organic phase was washed with brine (50 mL), dried with anhydrous Na₂SO₄, filtered and concentrated in vacuum to give a residue The residue was purified by silica gel chromatography (Petroleum ether/Ethyl acetate=10/1 to 1/2) to give compound CAT29 (530 mg, 630.40 umol, 35.19% yield, 99.6% purity) as a yellow oil.

### LCMS: [M+H]⁺: 838.3;

¹H NMR (400 MHz, CDCl₃) δ=4.88-4.83 (m, 2H), 4.25-3.81 (m, 1H), 3.11-2.86 (m, 5H), 2.33-2.30 (m, 6H), 2.10-1.97 (m, 4H), 1.58-1.50 (m, 23H), 1.32-1.22 (m, 40H), 0.90-0.87 (m, 15H).

### Example 1.30: Synthesis of CAT30

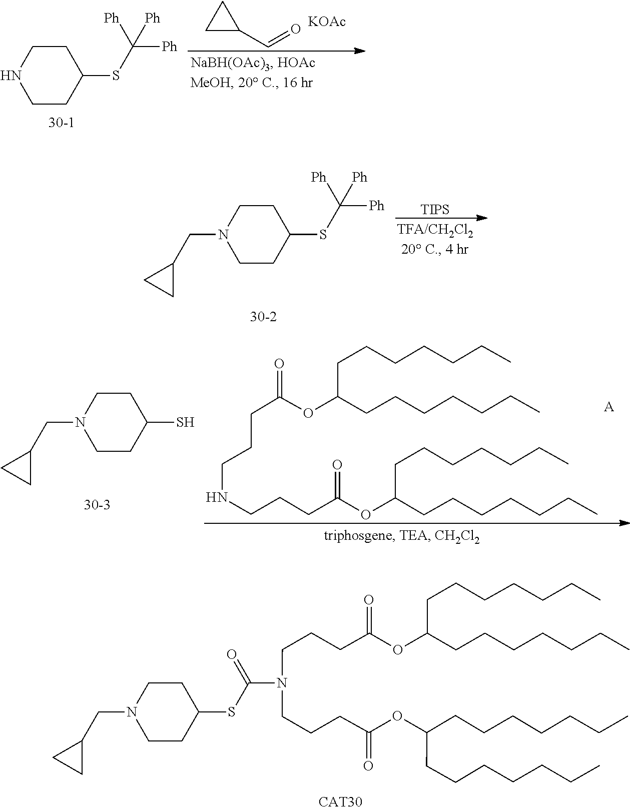

### Step 1: 1-(cyclopropylmethyl)-4-(tritylthio)piperidine (30-2)

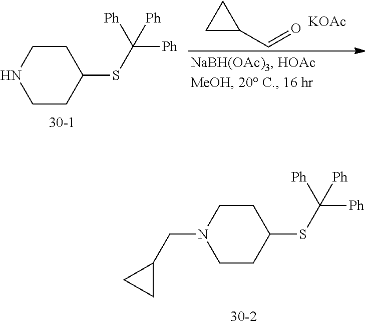

A mixture of 4-tritylsulfanylpiperidine (15 g, 31.68 mmol, 1 eq, TFA), cyclopropanecarbaldehyde (16.65 g, 95.03 mmol, 17.8 mL, 40% purity, 3 eq), HOAc (3.80 g, 63.35 mmol, 3.6 mL, 2 eq), KOAc (6.22 g, 63.35 mmol, 2 eq) in MeOH (50 mL) was degassed and purged with N₂ 3 times, the mixture was stirred at 20° C. for 2 hr under N₂ atmosphere. Then, NaBH(OAc)₃ (13.43 g, 63.35 mmol, 2 eq) was added. The resulting mixture was stirred at 20° C. for 14 hr. After completion, iced water (50 mL) was added and the mixture was neutralized to pH 8-9 with saturated NaHCO₃ solution. The aqueous phase was extracted with EtOAc (150 mL*3). The combined organic phase was washed with brine (100 mL), dried with anhydrous Na₂SO₄, filtered and concentrated in vacuum to give a crude product. The residue was purified by inverted MPLC (MeCN: H₂O: 0˜45%) to give compound 29-2 (7.2 g, 15.67 mmol, 50.1% yield, 90% purity) as a yellow solid.

### LCMS [M+H]⁺: 414.5

¹H NMR (400 MHz, CDCl₃) δ=7.56-7.39 (m, 6H), 7.38-7.30 (m, 9H), 3.65-3.48 (m, 2H), 3.02-2.79 (m, 4H), 2.47-2.31 (m, 3H), 2.28-2.17 (m, 2H), 1.43-1.37 (m, 1H), 1.25-1.01 (m, 1H), 0.83-0.76 (m, 2H), 0.45-0.35 (m, 2H).

### Step 2: 1-(cyclopropylmethyl)piperidine-4-thiol (29-3)

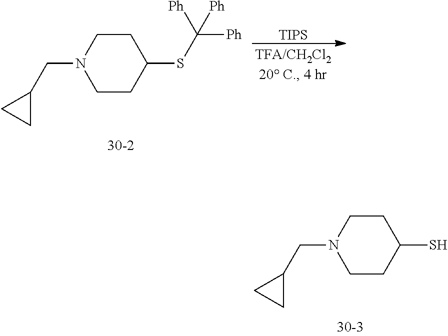 [SMILES: c1ccc(C(SC2CCN(CC3CC3)CC2)(c2ccccc2)c2ccccc2)cc1 ; SC1CCN(CC2CC2)CC1]

To a solution of 1-(cyclopropylmethyl)-4-tritylsulfanyl-piperidine (6 g, 14.51 mmol, 1 eq) in DCM (80 mL) were added TFA (30.80 g, 270.13 mmol, 20 mL, 18.62 eq) and TIPS (5.69 g, 29.01 mmol, 2 eq) at 0° C. under N₂. After addition, the resulting mixture was stirred at 20° C. for 4 hr. After completion, the reaction mixture was concentrated under reduced pressure to remove TFA and filtered. The filtrate was diluted with MeOH (150 mL) and extracted with PE (50 mL*5). The MeOH layers was concentrated under reduced pressure to give compound 30-3 (4.1 g, crude, TFA) as a yellow oil. The crude product was used in the next step without further purification.

### Step 3: di(pentadecan-8-yl) 4,4′-((((1-(cyclopropylmethyl)piperidin-4-yl)thio)carbonyl)azanediyl)dibutanoate (CAT30)

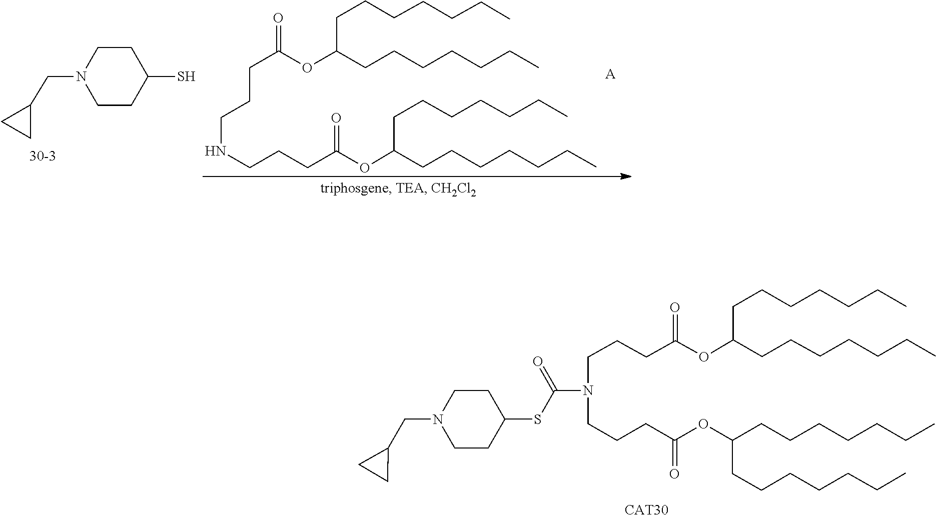

To a solution of 1-heptyloctyl 4-[[4-(1-heptyloctoxy)-4-oxo-butyl]amino]butanoate (1.8 g, 2.95 mmol, 1 eq) dissolved in dry CH₂Cl₂ (30 mL) were added TEA (895.78 mg, 8.85 mmol, 1.23 mL, 3 eq) and triphosgene (525.39 mg, 1.77 mmol, 0.6 eq) at 0° C. under N₂. The resulting solution was stirred at 20° C. for 1 hr. The resulting reaction was concentrated under reduced pressure. To a solution of 1-(cyclopropylmethyl)piperidine-4-thiol (2.53 g, 8.85 mmol, 3 eq, TFA) dissolved in dry THF (25 mL) was added NaOH (826.22 mg, 20.66 mmol, 7 eq) at 0° C. under N₂. To this resulting solution, carbamoyl chloride, dissolved in THF (20 mL), was added via syringe slowly under N₂ at 0° C. The resulting solution was stirred at 20° C. for 15 hr. After completion, the reaction mixture was quenched by NH₄Cl (80 mL) at 0° C. and then diluted with EtOAc (50 mL). The aqueous phase was extracted with EtOAc (60 mL* 3). The combined organic phase was washed with brine (50 mL), dried with anhydrous Na₂SO₄, filtered and concentrated in vacuum to give a residue. The residue was purified by flash silica gel chromatography (20 g SepaFlash® Silica Flash Column, EtOAc: PE: 0˜20%) and positive prep-HPLC (column: Welch Ultimate XB-SiOH 250*50*10 um; mobile phase: [Hexane-EtOH]; B %: 0%-20%, 10 min) to yield CAT30 (235 mg, 0.28 mmol, 44.3% yield, 98% purity) as a light yellow oil.

### LCMS [M+H]⁺: 808.4

¹H NMR (400 MHz, CDCl₃) δ=4.89-4.85 (m, 2H), 3.48-3.31 (m, 5H), 2.97-2.93 (m, 2H), 2.33-2.28 (m, 4H), 2.25-2.18 (m, 4H), 2.08-2.01 (m, 2H), 1.91-1.87 (m, 4H), 1.77-1.68 (m, 3H), 1.55-1.18 (m, 8H), 1.32-1.26 (m, 40H), 0.91-0.86 (m, 12H), 0.53-0.50 (m, 2H), 0.11-0.08 (m, 2H).

### Example 1.31: Synthesis of CAT31

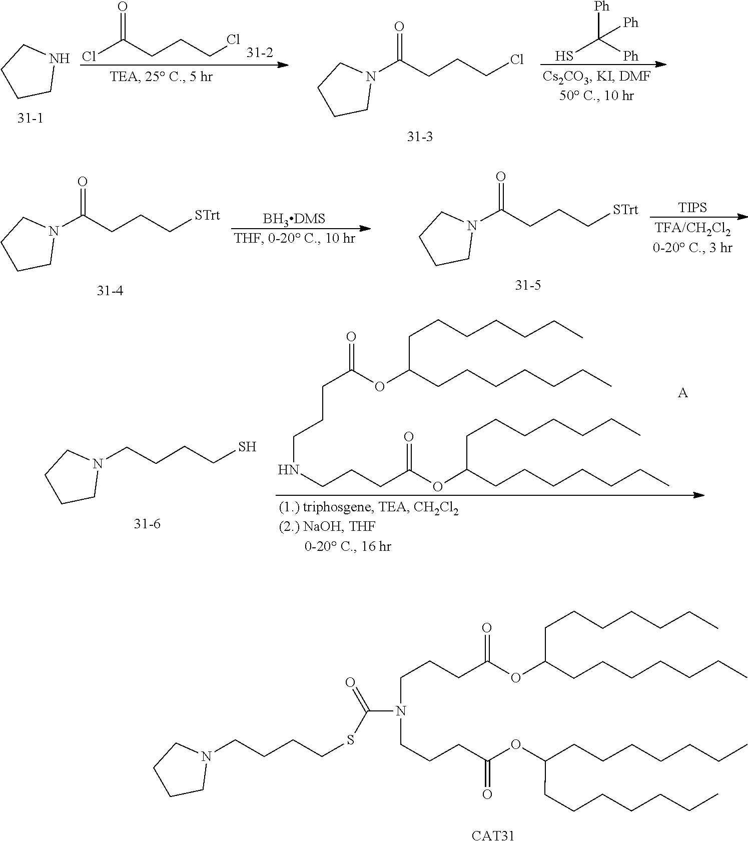

### Step 1: 4-chloro-1-(pyrrolidin-1-yl)butan-1-one (31-3)

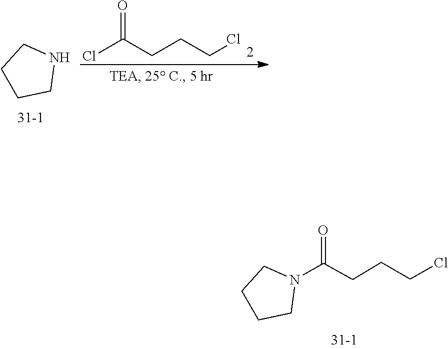

To a solution of pyrrolidine (5.00 g, 70.3 mmol, 5.87 mL, 1.00 eq) in THF (120 mL) was added TEA (14.2 g, 141 mmol, 19.6 mL, 2.00 eq), then 4-chlorobutanoyl chloride (11.9 g, 84.4 mmol, 9.44 mL, 1.20 eq) was added slowly. The mixture was stirred at 25° C. for 5 hours. The reaction mixture was quenched by the addition of water (100 mL) at 25° C., and then extracted with EtOAc (100 mL×3). The combined organic layers were washed with brine (100 mL×2), dried over anhydrous sodium sulfate, filtered and concentrated under reduced pressure to give a residue. The residue was purified by column chromatography (SiO₂, Petroleum ether/EtOAc=I/O to 3/1). Compound 31-3 (5.60 g, 28.3 mmol, 40.2% yield, 88.8% purity) was obtained as a yellow oil.

### LCMS [M+1]⁺: 175.9.

¹H NMR (400 MHz, CDCl₃) δ=3.64 (t, J=6.0 Hz, 2H), 3.46-3.40 (m, 4H), 2.43 (t, J=6.8 Hz, 2H), 2.115-2.09 (m, 2H), 1.98-1.91 (m, 2H), 1.88-1.81 (m, 2H).

### Step 2: 1-(pyrrolidin-1-yl)-4-(tritylthio)butan-1-one (31-4)

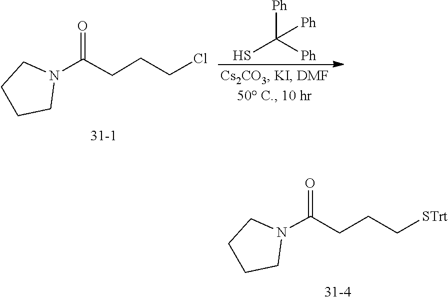

A mixture of 4-chloro-1-pyrrolidin-1-yl-butan-1-one (5.00 g, 28.5 mmol, 1.00 eq), triphenylmethanethiol (9.44 g, 34.2 mmol, 1.20 eq), K₂C₀₃ (15.7 g, 114 mmol, 4.00 eq), KI (473 mg, 2.85 mmol, 0.10 eq) in DMF (50 mL) was degassed and purged with N₂ 3 times, and then the mixture was stirred at 50° C. for 10 hours under N₂ atmosphere. The reaction mixture was partitioned between EtOAc (100 mL) and H₂O (100 mL). The organic phase was separated, washed with brine (60 mL×3), dried over anhydrous sodium sulfate, filtered and concentrated under reduced pressure to give a residue. The residue was purified by flash silica gel chromatography (ISCO®; 80 g SepaFlash® Silica Flash Column, Eluent of 0˜10% EtOAc/Petroleum ethergradient @ 100 mL/min). Compound 31-4 (9.32 g, 17.7 mmol, 62.2% yield, 79.0% purity) was obtained as a white solid.

### LCMS [2M+1]⁺: 831.4.

¹H NMR (400 MHz, CDCl₃) δ=7.37-7.32 (m, 6H), 7.23-7.18 (m, 6H), 7.16-7.11 (m, 3H), 3.33 (t, J=6.8 Hz, 2H), 3.25 (t, J=6.8 Hz, 2H), 2.18 (t, J=6.8 Hz, 2H), 2.13 (t, J=7.6 Hz, 2H), 1.87-1.81 (m, 2H), 1.78-1.73 (m, 2H), 1.67 (t, J=7.6 Hz, 2H).

### Step 3: 1-(4-(tritylthio)butyl)pyrrolidine (31-5)

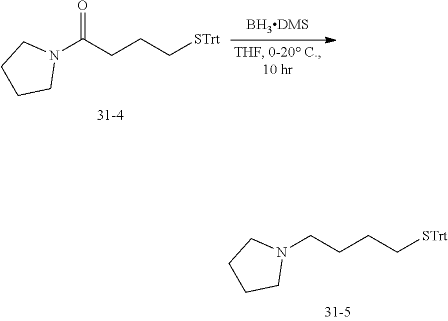

To a solution of 1-pyrrolidin-1-yl-4-tritylsulfanyl-butan-1-one (9.00 g, 21.7 mmol, 1.00 eq) in THF (120 mL) was added BH₃-Me₂S (10.0 M, 10.8 mL, 5.00 eq) at 0° C. via syringe, dropwise, under N₂ atmosphere, then the mixture was stirred at 20° C. for 10 hours under N₂ atmosphere. The reaction was quenched by methanol (100 mL) and concentrated. Then the residue was diluted with EtOAc (100 mL) and H₂O (100 mL), extracted with EtOAc (100 mL×3), washed by brine (200 mL), dried over anhydrous sodium sulfate, filtered and concentrated under reduced pressure to give a residue. The aqueous phase was quenched by sodium hypochlorite solution and discarded. Compound 31-5 (7.60 g, 12.5 mmol, 57.7% yield, 66.0% purity) was obtained as a yellow solid.

¹H NMR (400 MHz, CDCl₃) δ=7.39-7.37 (m, 6H), 7.27-7.23 (m, 6H), 7.20-7.16 (m, 3H), 3.13-3.08 (m, 2H), 2.63-2.51 (m, 4H), 2.17-2.10 (m, 4H), 1.82-1.79 (m, 2H), 1.75-1.69 (m, 2H), 1.33-1.25 (m, 2H).

### Step 4: 4-(pyrrolidin-1-yl)butane-1-thiol (31-6)

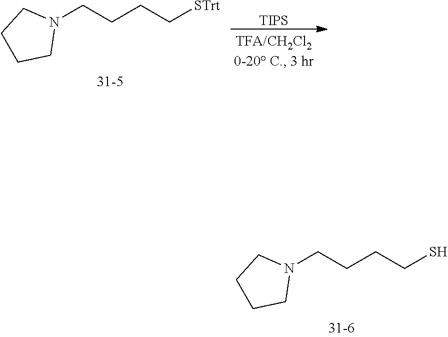

A mixture of 1-(4-tritylsulfanylbutyl)pyrrolidine (5.00 g, 12.5 mmol, 1.00 eq) in TFA (16.0 mL) and DCM (52.0 mL) was degassed and purged with N₂ 3 times, then triisopropylsilane (3.94 g, 24.9 mmol, 5.11 mL, 2.00 eq) was added slowly at 0° C. The mixture was stirred at 20° C. for 3 hours under N₂ atmosphere. The reaction mixture was concentrated under reduced pressure. The mixture was diluted with methanol (50 mL) and washed with petroleum ether (60 mL×5). The methanol layer was concentrated under reduced pressure to give compound 31-6 (3.40 g, crude, TFA salt) as a yellow oil.

¹H NMR (400 MHz, CDCl₃) δ=3.34-3.28 (m, 1H), 3.15-3.10 (m, 1H), 2.95-2.82 (m, 4H), 2.60-2.54 (m, 2H), 2.13-2.08 (m, 2H), 1.94-1.78 (m, 3H), 1.72-1.62 (m, 2H), 1.41-1.35 (m, 1H).

### Step 5: di(pentadecan-8-yl) 4,4′-((((4-(pyrrolidin-1-yl)butyl)thio)carbonyl)azanediyl)dibutanoate (CAT31)

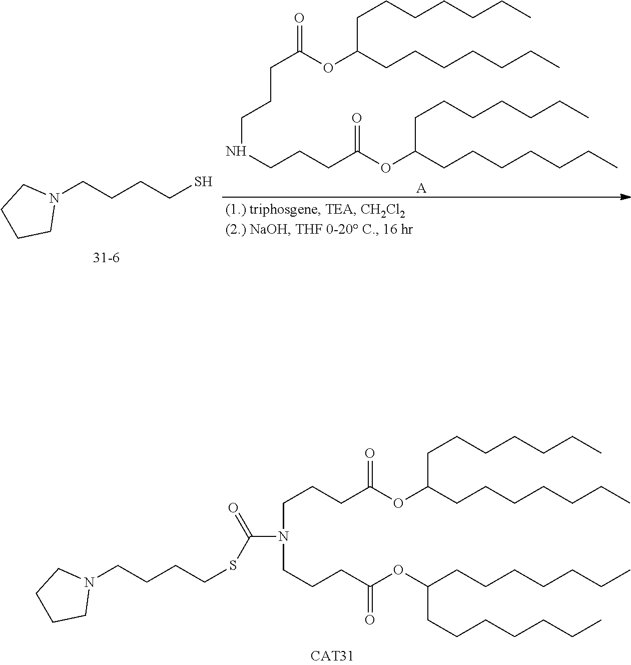

To a solution of 1-heptyloctyl 4-[[4-(1-heptyloctoxy)-4-oxo-butyl]amino]butanoate (2.00 g, 3.28 mmol, 1.00 eq) in dry dichloromethane (25.0 mL) were added TEA (995 mg, 9.84 mmol, 1.37 mL, 3.00 eq) and triphosgene (920 mg, 3.10 mmol, 0.90 eq) at 0° C. under N₂ atmosphere. The resulting solution was stirred at 20° C. for 1 hour. The resulting reaction was concentrated under reduced pressure. To a solution of 4-pyrrolidin-1-ylbutane-1-thiol (3.14 g, 11.5 mmol, 3.50 eq, TFA salt) in dry THF (25.0 mL) was added NaOH (1.31 g, 32.8 mmol, 10.0 eq) at 0° C. under N₂ atmosphere. To this resulting solution was added carbamoyl chloride in THF (15.0 mL) at 0° C. under N₂ atmosphere. The resulting solution was stirred at 20° C. for 15 hours. The reaction mixture was quenched by NH₄Cl (60.0 mL) at 0° C. and then diluted with EtOAc (60.0 mL). The aqueous phase was extracted with EtOAc (60.0 mL×3). The combined organic phase was washed with brine (70.0 mL), dried with anhydrous sodium sulfate, filtered and concentrated in vacuum to give a residue. The residue was purified by flash silica gel chromatography (ISCO®; 120 g SepaFlash® Silica Flash Column, Eluent of 0˜50% EtOAc/Petroleum ethergradient @ 100 mL/min), then was purified by positive prep-HPLC(column: Welch Ultimate XB -CN 250*50*10 um; mobile phase: [Hexane -EtOH]; B %: 0%-35%, 20 min). Compound CAT31 (260 mg, 0.322 mmol, 10.2% yield, 98.5% purity) was obtained as a yellow oil.

### LCMS [M+1]⁺: 796.1.

¹H NMR (400 MHz, CDCl₃) δ=4.90-4.84 (m, 2H), 3.58-3.53 (m, 1H), 3.38-3.34 (m, 4H), 3.26-3.20 (m, 1H), 2.95-2.89 (m, 2H), 2.53-2.52 (m, 2H), 2.49-2.46 (m, 2H), 2.33-2.29 (m, 4H), 2.14-2.07 (m, 1H), 2.00-1.97 (m, 1H), 1.89-1.87 (m, 4H), 1.80-1.77 (m, 2H), 1.65-1.63 (m, 2H), 1.52-1.51 (m, 8H), 1.32-1.27 (m, 42H), 0.88 (t, J=6.4 Hz, 12H).

### Example 1.32: Synthesis of CAT32

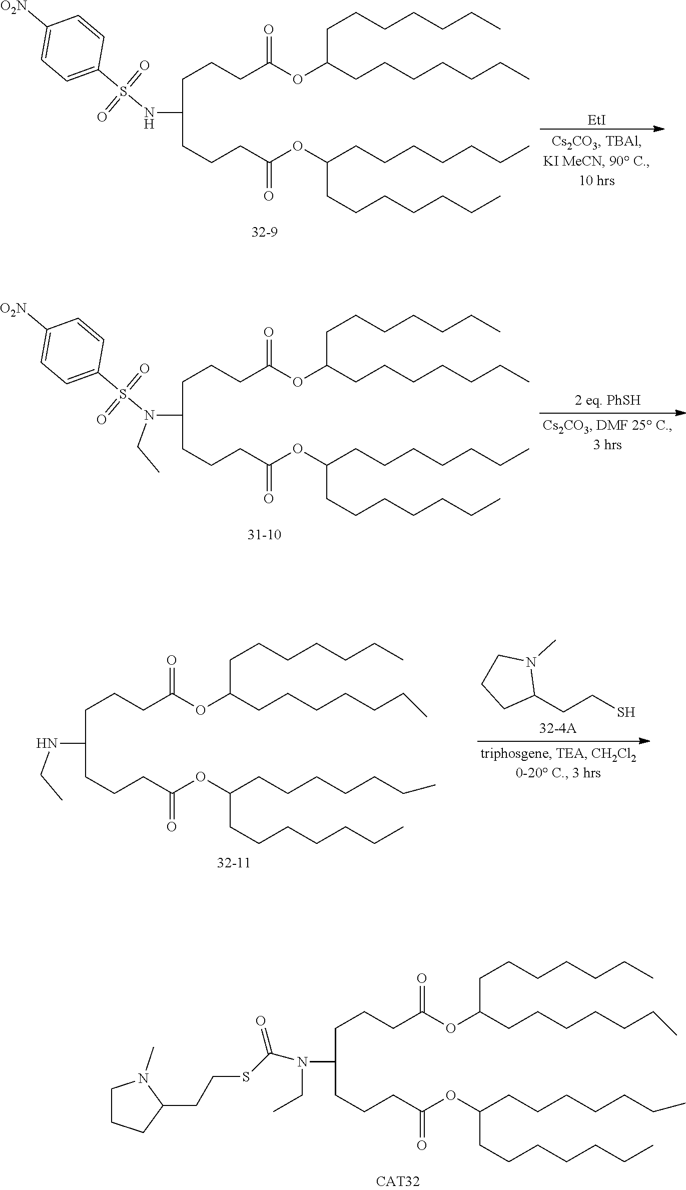

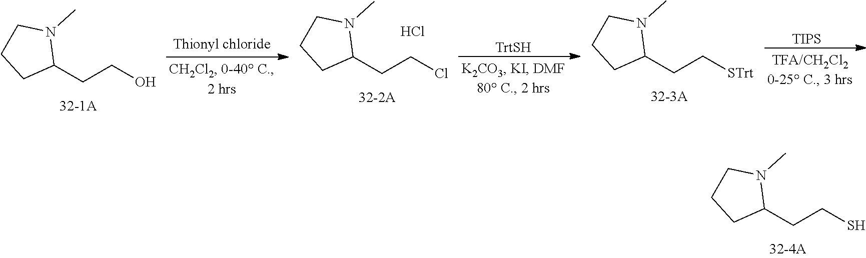

### Step 1: di(pentadecan-8-yl) 5-(N-ethyl-4-nitrophenylsulfonamido)nonanedioate (32-10)

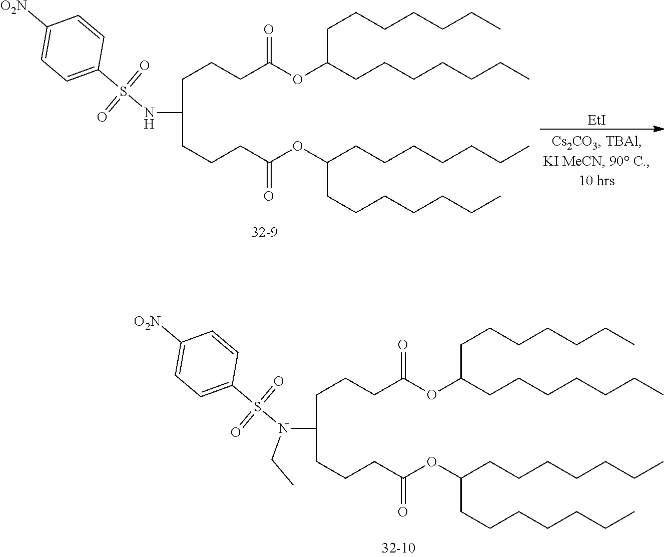 [SMILES: "EtI" ; CCCCCCCC(CCCCCCC)OC(=O)CCCC(CCCC(=O)OC(CCCCCCC)CCCCCCC)NS(=O)(=O)c1ccc([N+](=O)[O-])cc1 ; CCCCCCCC(CCCCCCC)OC(=O)CCCC(CCCC(=O)OC(CCCCCCC)CCCCCCC)N(CC)S(=O)(=O)c1ccc([N+](=O)[O-])cc1]

To a solution of di(pentadecan-8-yl) 5-(4-nitrophenylsulfonamido)nonanedioate (5.00 g, 6.18 mmol, 1 eq) and iodoethane (1.16 g, 7.41 mmol, 0.593 mL, 1.2 eq) in MeCN (50 mL) were added Cs₂CO₃ (6.04 g, 18.5 mmol, 3 eq), TBAI (22.8 mg, 61.8 umol, 0.01 eq) and KI (513 mg, 3.09 mmol, 0.5 eq). The mixture was stirred at 90° C. for 10 hours. The reaction mixture was filtered. The filtrate was diluted with water (50 mL), extrated with and ethyl acetate (30 mL×3). The combined organic layers were washed with brine(30 mL), dried over Na₂SO₄, filtered and concentrated under reduced pressure to give a residue. The residue was purified by flash silica gel chromatography (ISCO®; 120 g SepaFlash® Silica Flash Column, Eluent of 0˜10% Ethyl acetate/Petroleum ethergradient @ 100 mL/min) to give di(pentadecan-8-yl) 5-(N-ethyl-4-nitrophenylsulfonamido)nonanedioate (4.20 g, 4.67 mmol, 75.5% yield, 93% purity) as a yellow oil.

### LCMS [M+23]+: 859.5

¹H NMR (400 MHz, CDCl₃) δ=8.35 (d, J=8.8 Hz, 2H), 8.03 (d, J=8.8 Hz, 2H), 4.86-4.80 (m, 2H), 3.23-3.18 (m, 2H), 2.28-2.19 (m, 4H), 1.54-1.44 (m, 17H), 1.26-1.23 (s, 40H), 0.90-0.87 (m, 15H).

### Step 2: di(pentadecan-8-yl) 5-(ethylamino)nonanedioate (31-11)

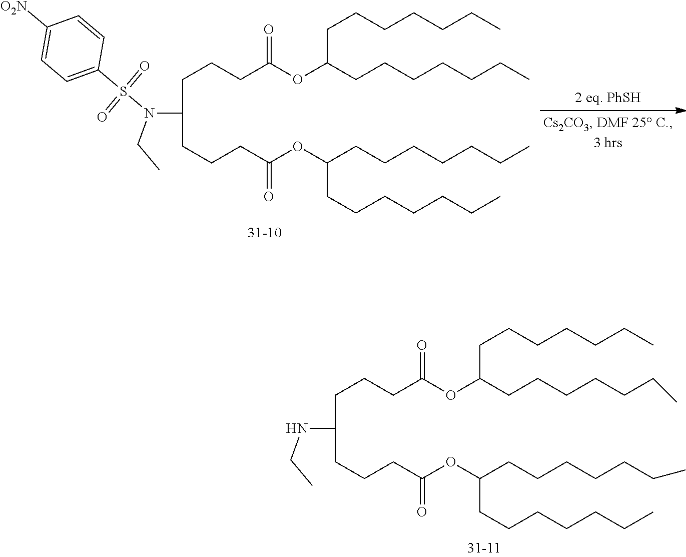 [SMILES: CCCCCCCC(CCCCCCC)OC(=O)CCCC(CCCC(=O)OC(CCCCCCC)CCCCCCC)N(CC)S(=O)(=O)c1ccc([N+](=O)[O-])cc1 ; CCCCCCCC(CCCCCCC)OC(=O)CCCC(CCCC(=O)OC(CCCCCCC)CCCCCCC)NCC]

To a solution of di(pentadecan-8-yl) 5-(N-ethyl-4-nitrophenylsulfonamido)nonanedioate (4.20 g, 5.02 mmol, 1 eq) and Cs₂CO₃ (3.27 g, 10.0 mmol, 2 eq) in DMF (50 mL) was added benzenethiol (1.67 g, 15.2 mmol, 1.55 mL, 3.02 eq) and then the mixture was stirred at 25° C. for 3 hours under N₂ atmosphere. The reaction mixture was quenched by the addition of water (100 mL), and then extracted with ethyl acetate (200 mL×3). The combined organic layers were washed with brine (100 mL), dried over Na₂SO₄, filtered and concentrated under reduced pressure. The residue was purified by flash silica gel chromatography (ISCO®; 120 g SepaFlash® Silica Flash Column, Eluent of 0˜50% Ethyl acetate/Petroleum ethergradient @ 100 mL/min) to give di(pentadecan-8-yl) 5-(ethylamino)nonanedioate (2.50 g, 3.83 mmol, 76.4% yield) as a yellow oil.

¹H NMR (400 MHz, CDCl₃) δ=4.90-4.84 (m, 2H), 2.65-2.60 (m, 2H), 2.54-2.52 (m, 1H), 2.30 (t, J=7.6 Hz, 4H), 1.61-1.40 (m, 16H), 1.27-1.24 (m, 40H), 1.11 (t, J=7.2 Hz, 3H), 0.95-0.87 (m, 12H).

### Step 3: 2-(2-chloroethyl)-1-methylpyrrolidine (32-2A)

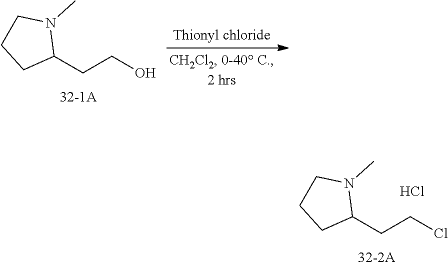

To a solution of 2-(1-methylpyrrolidin-2-yl)ethanol (45.0 g, 348 mmol, 47.3 mL, 1 eq) in CH₂Cl₂ (500 mL) was added SOCl₂ (124 g, 1.04 mol, 75.8 mL, 3 eq) dropwise slowly at 0° C. Then the mixture was stirred at 40° C. for 2 hours. The reaction mixture was filtered and concentrated under reduced pressure to give compounds 32-2A (53.0 g, crude, HCl) as a brown solid. The compound was used directly for the next step.

¹H NMR (400 MHz, DMSO-d₆) δ=11.13 (s, 1H), 3.84-3.80 (m, 1H), 3.71-3.64 (m, 1H), 3.52-3.48 (m, 1H), 3.39-3.30 (m, 1H), 3.06-2.97 (m, 1H), 2.75 (d, J=4.8 Hz, 3H), 2.37-2.33 (m, 1H), 2.24-2.11 (m, 2H), 1.99-1.84 (m, 2H), 1.74-1.64 (m, 1H).

### Step 4: 1-methyl-2-(2-(tritylthio)ethyl)pyrrolidine (32-3A)

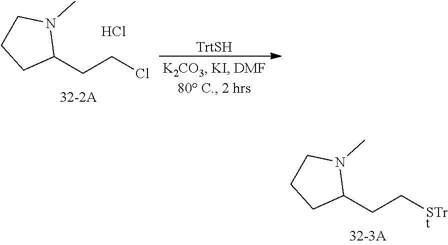

To a solution of 2-(2-chloroethyl)-1-methylpyrrolidine (53.0 g, 359 mmol, 1 eq) and triphenylmethanethiol (119 g, 431 mmol, 1.2 eq) in DMF (500 mL) were added K₂CO₃ (198 g, 1.44 mol, 4 eq) and KI (5.96 g, 35.9 mmol, 0.1 eq). The mixture was stirred at 80° C. for 2 hours. The reaction mixture was filtered and extracted with water (1000 mL) and EtOAc (300×3 mL). The combined organic layers were washed with brine, dried over Na₂SO₄, filtered and concentrated under reduced pressure to give a residue. The residue was purified by flash silica gel chromatography (ISCO®; 330 g SepaFlash® Silica Flash Column, Eluent of 0˜50% Ethyl acetate/Petroleum ethergradient @ 100 mL/min) to give compound 32-3A (12.0 g, 30.7 mmol, 18.3% yield, 99% purity) as a yellow oil.

### LCMS [M+1]⁺: 388.2.

¹H NMR (400 MHz, CDCl₃) δ=7.44-7.41 (m, 6H), 7.31-7.27 (m, 6H), 7.23-7.20 (m, 3H), 2.20 (s, 3H), 2.18-2.08 (m, 2H), 1.98-1.92 (m, 1H), 1.78-1.71 (m, 3H), 1.65-1.56 (m, 2H), 1.35-1.30 (M, 1H), 1.23-1.15 (m, 1H).

### Step 5: 2-(I-methylpyrrolidin-2-yl)ethanethiol (32-4A)

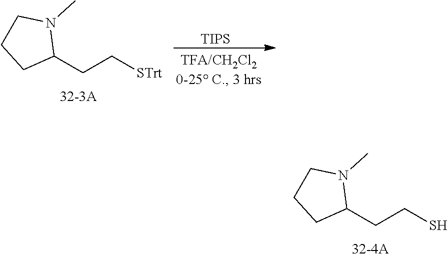

To a solution of 1-methyl-2-(2-(tritylthio)ethyl)pyrrolidine (5.50 g, 14.2 mmol, 1 eq) in TFA (10 mL) and CH₂Cl₂ (30 mL) was added triisopropylsilane (4.49 g, 28.4 mmol, 5.83 mL, 2 eq) at 0° C. The mixture was stirred at 25° C. for 3 hours. The reaction mixture was concentrated under reduced pressure to give a residue, the residue was dissolved in methanol (10 mL), and extracted with petroleum ether (10 mL×5). The combined methanol layers was concentrated under reduced pressure to give compound 32-4A (3.68 g, crude, TFA) was obtained as a yellow oil. The compound was used directly for the next step.

¹H NMR (400 MHz, DMSO-d₆) δ=3.57 (m, 1H), 3.33 (m, 1H), 3.06 (m, 1H), 2.82 (s, 3H), 2.64 (d, J=15.8 Hz, 2H), 2.24 (m, 1H), 2.16-2.04 (m, 1H), 1.98 (m, 1H), 1.93-1.71 (m, 2H), 1.62 (m, 1H).

### Step 6: di(pentadecan-8-yl) 5-(ethyl(((2-(I-methylpyrrolidin-2-yl)ethyl)thio)carbonyl)amino)nonanedioate (CAT32)

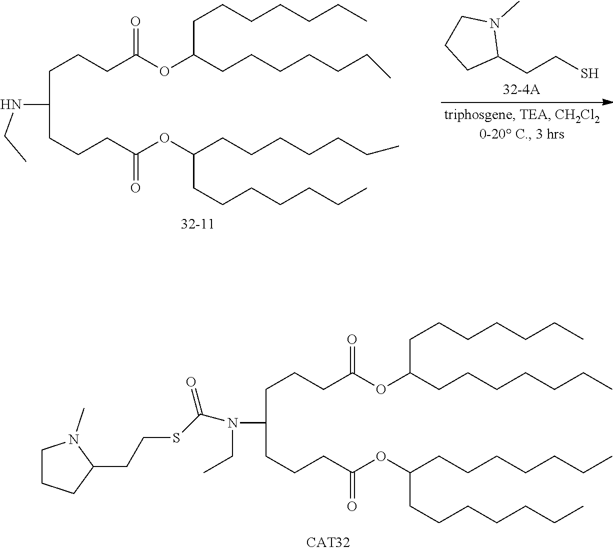

To a solution of di(pentadecan-8-yl) 5-(ethylamino)nonanedioate (2.50 g, 3.83 mmol, 1 eq) dissolved in dry CH₂Cl₂ (20 mL) were added TEA (1.16 g, 11.5 mmol, 1.60 mL, 3 eq) and triphosgene (1.07 g, 3.61 mmol, 0.94 eq) at 0° C. under N₂. The resulting solution was stirred at 20° C. for 1 hour. The resulting reaction was concentrated under reduced pressure. To a 2-(1-methylpyrrolidin-2-yl)ethanethiol (3.48 g, 13.4 mmol, 3.5 eq, TFA) in dry THF (30 mL) at 0° C. was added NaOH (1.53 g, 38.34 mmol, 10 eq) under nitrogen atmosphere. To this resulting solution was added carbamoyl chloride in THF (10 mL) via syringe at 0° C. under N₂. The resulting solution was stirred at 20° C. for 2 hours. The reaction mixture was quenched by NH₄Cl (50 mL) at 0° C. and then diluted with ethyl acetate (50 mL). The aqueous phase was extracted with ethyl acetate (50 mL×3). The combined organic phase was washed with brine (30 mL), dried with anhydrous Na₂SO₄, filtered and concentrated in vacuum to give a residue. The residue was purified by flash silica gel chromatography (ISCO®; 120 g SepaFlash® Silica Flash Column, Eluent of 0-50% Ethyl acetate/Petroleum ethergradient @ 100 mL/min) and prep-HPLC (column: Welch Ultimate C_{18 150*25} mm*5 um; mobile phase: [water(HCl)—MeOH];B %: 70%-100%, 10 min) to give CAT32 (400 mg, 480.48 umol, 73.26% yield, 98.9% purity) as a yellow oil.

### LCMS [M+1]⁺: 824.4.

¹H NMR (400 MHz, CDCl₃) δ=4.88-4.85 (m, 2H), 4.30-3.82 (m, 1H), 3.27-3.26 (m, 2H), 3.05-3.03 (m, 1H), 2.89-3.00 (m, 1H), 2.78-2.89 (m, 1H), 2.32 (s, 3H), 2.31-2.25 (m, 3H), 2.16-2.12 (m, 2H), 2.00-1.97 (m, 2H), 1.59-1.50 (m, 21H), 1.27-1.25 (m, 43H), 0.90-0.87 (m, 12H).

### Example 1.33: Synthesis of CAT33

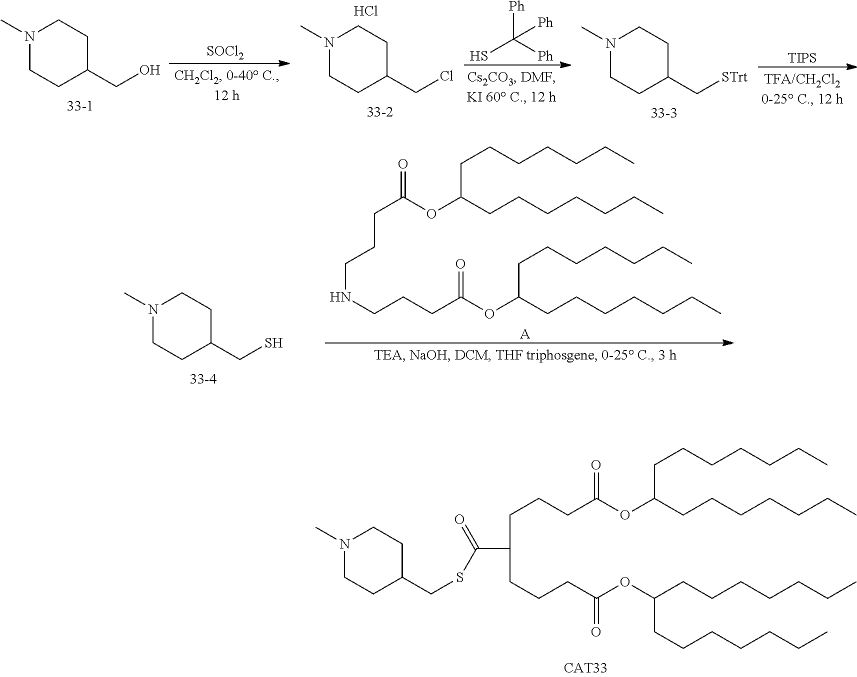

### Step 1: 4-(chloromethyl)-1-methyl-piperidine (33-2)

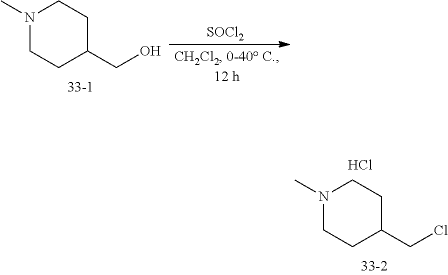

To a solution of (1-methyl-4-piperidyl) methanol (20 g, 154.80 mmol, 1 eq) in CH₂Cl₂ (200 mL) was added SOCl₂ (22.10 g, 185.76 mmol, 13.48 mL, 1.2 eq) at 0° C. The mixture was stirred at 40° C. for 12 hr. The reaction mixture was concentrated under reduced pressure to give compound 33-2 (20 g, 108.63 mmol, 70.18% yield) as a brown solid.

¹H NMR (400 MHz, DMSO-d₆) δ=10.96 (s, 1H), 3.56 (d, J=5.6 Hz, 2H), 3.67-3.33 (m, 2H), 2.97-2.94 (m, 2H), 2.68 (s, 3H), 1.97-1.62 (m, 5H).

### Step 2: 1-methyl-4-(tritylsulfanylmethyl)piperidine (33-3)

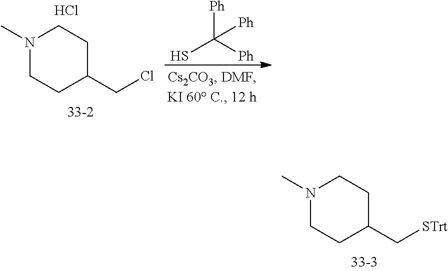

To a solution of 4-(chloromethyl)-1-methyl-piperidine (20 g, 108.63 mmol, 1 eq) and triphenylmethanethiol (45.04 g, 162.95 mmol, 1.5 eq) in DMF (200 mL) were added Cs₂CO₃ (70.79 g, 217.27 mmol, 2 eq) and KI (9.02 g, 54.32 mmol, 0.5 eq). The mixture was stirred at 60° C. for 12 hr. The reaction mixture was diluted with water (300 mL×3) and extracted with EtOAC (400 mL×3). The combined organic layers were washed with brine, dried over sodium sulfate, filtered and concentrated under reduced pressure. The residue was purified by column chromatography (PE/EtOAc=20/1 to 0/1) to give compound 33-3(17 g, 43.86 mmol, 40.38% yield) as a brown oil.

¹H NMR (400 MHz, CDCl₃) (7.50-7.47 (m, 6H), 7.33-7.27 (m, 9H), 2.64-2.58 (m, 2H), 2.32 (s, 3H), 1.91-1.88 (m, 2H), 1.66-1.61 (m, 3H), 1.44-1.28 (m, 4H).

### Step 3: (1-methyl-4-piperidyl)methanethiol: (33-4)

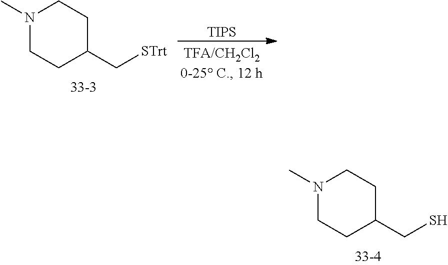

To a solution of 1-methyl-4-(tritylsulfanylmethyl)piperidine (17 g, 43.86 mmol, 1 eq) and triisopropylsilane (20.84 g, 131.59 mmol, 27.03 mL, 3 eq) in CH₂Cl₂ (200 mL), and then TFA (32.73 g, 287.00 mmol, 21.25 mL, 6.54 eq) was added at 0° C. The mixture was stirred at 25° C. for 12 hr. The reaction mixture was concentrated under reduced pressure to remove TFA, it was diluted with methanol (300 mL×3) and washed with PE (200 mL×3). The combined organic layers were dried over sodium sulfate, the methanol layers was concentrated under reduced pressure to give compound 33-4 (4 g, 27.54 mmol, 62.78% yield) as a brown oil.

### Step 4: 1-heptyloctyl 4-[[4-(1-heptyloctoxy)-4-oxo-butyl]-[(1-methyl-4-piperidyl)methylsulfanylcarbonyl]amino]butanoate: (CAT33)

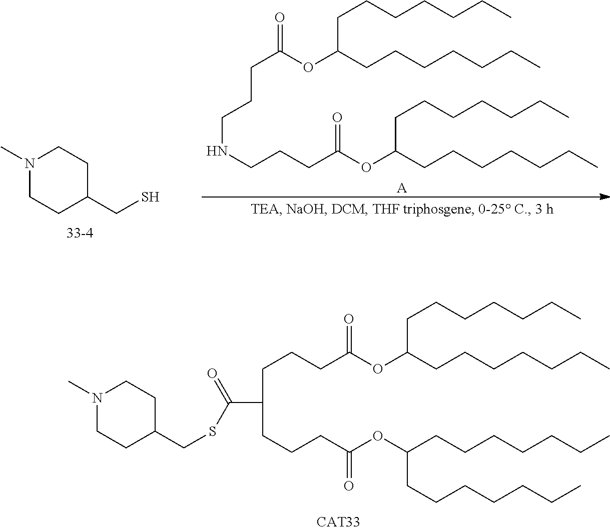

To a solution of 1-heptyloctyl 4-[[4-(1-heptyloctoxy)-4-oxo-butyl]amino]butanoate (3 g, 4.92 mmol, 1 eq) dissolved in dry CH₂Cl₂ (30 mL) were added TEA (1.49 g, 14.75 mmol, 2.05 mL, 3 eq) and triphosgene (729.70 mg, 2.46 mmol, 0.5 eq) at 0° C. under N₂ atmosphere. The resulting solution was stirred at 20° C. under N 2 for 1 hr. The reaction was concentrated under reduced pressure and kept under N 2. NaOH (1.38 g, 34.43 mmol, 7 eq) was dissolved in dry THF (50 mL) at 0° C. under N₂, then (1-methyl-4-piperidyl)methanethiol (3.57 g, 24.59 mmol, 5 eq) was added. To this resulting solution, carbamoyl chloride dissolved in THF (50 mL) was added slowly under N₂ at 0° C. The mixture was stirred at 25° C. for 2 hr. The reaction mixture was quenched with saturated aqueous NH₄C₁ (100 mL) and then diluted with EtOAC (100 mL). The aqueous phase was extracted with EtOAC (100 mL×3). The combined organic phase was washed with brine (50 mL), dried with anhydrous sodium sulfate, filtered and concentrated in vacuum to give a residue. The residue purified by silica gel chromatography (PE/EtOAc=10/1 to 1/2) and purified by prep-HPLC (column: Welch Ultimate C_{18 150*25} mm*5 um; mobile phase: [water(HCl)-MeOH]; B %: 70%-100%, 10 min) to give compound CAT33 (137.8 mg, 184.39 umol, 62.63% yield, 98% purity) as a yellow oil.

### LCMS: [M+H]⁺: 781.6

¹H NMR (400 MHz, CDCl₃) (5 4.88-4.86 (m, 2H), 3.54-3.51 (m, 2H), 3.46-3.38 (m, 5H), 2.83-2.85 (m, 2H), 2.75 (s, 3H), 2.67-2.62 (m, 2H), 2.40-2.32 (m, 4H), 2.01-1.89 (m, 8H), 1.55-1.51 (m, 8H), 1.35-1.27 (m, 40H), 0.90-0.84 (m, 12H).

### Example 1.34: Synthesis of CAT34

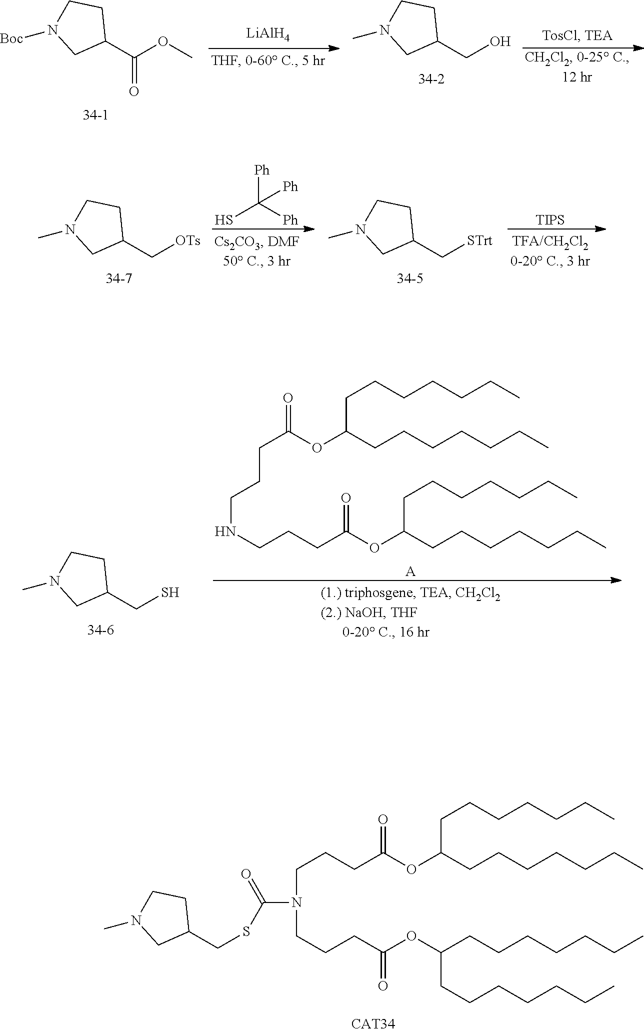

### Step 1: (1-methylpyrrolidin-3-yl)methanol (34-2)

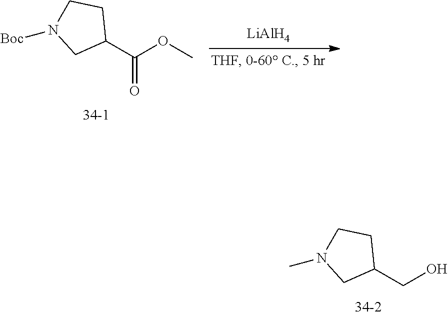

To a solution of 01-tert-butyl 03-methyl pyrrolidine-1,3-dicarboxylate (20.0 g, 87.2 mmol, 1.00 eq) in THF (350 mL) was added LiAlH₄ (8.28 g, 218 mmol, 2.50 eq) in portion at 0° C. under N₂. The mixture was stirred at 60° C. for 5 hours under N₂. The reaction mixture was quenched by the addition of water (8 mL) at 0° C. and 15% of NaOH solution (8 mL), then water (24 mL) was added slowly, the mixture stirred for 30 min, dried over anhydrous sodium sulfate, the filtered cake washed with EtOAc (100 mL×3), the filtrate concentrated under reduced pressure to give compound 34-2 (18.3 g, crude) as a yellow oil.

¹H NMR (400 MHz, DMSO-d₆) δ=4.51 (s, 1H), 3.30-3.22 (m, 2H), 2.44-2.29 (m, 3H), 2.25-2.21 (m, 1H), 2.19 (s, 3H), 2.17-2.10 (m, 1H), 1.82-1.73 (m, 1H), 1.37-1.29 (m, 1H).

### Step 2: (1-methylpyrrolidin-3-yl)methyl 4-methylbenzenesulfonate (34-7)

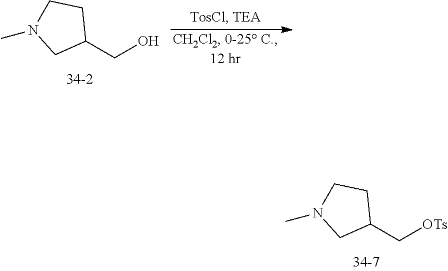

A mixture of (1-methylpyrrolidin-3-yl)methanol (18.0 g, 156 mmol, 1.00 eq), TEA (31.6 g, 313 mmol, 43.5 mL, 2.00 eq) and DMAP (1.91 g, 15.6 mmol, 0.10 eq) in CH₂Cl₂ (250 mL) was degassed and purged with N₂ 3 times, TosCl (44.7 g, 234 mmol, 1.50 eq) was added slowly at 0° C., and then the mixture was stirred at 25° C. under N₂ for 12 hours. The residue was diluted with CH₂Cl₂ (100 mL). The combined organic layers were washed with water (350 mL) and brine (250 mL), dried over anhydrous sodium sulfate, filtered and concentrated under reduced pressure to give a residue. The residue was purified by flash silica gel chromatography (ISCO®; 330 g SepaFlash® Silica Flash Column, Eluent of 0˜100% EtOAc/PE gradient @100 mL/min) to give compound 34-7 (12.7 g, 47.2 mmol, 30.2% yield) as a yellow oil.

### LCMS [M+1]⁺: 270.1.

¹H NMR (400 MHz, CDCl₃) δ=7.79-7.77 (m, 2H), 7.34 (d, J=8.0 Hz, 2H), 3.93 (d, J=7.2 Hz, 2H), 2.55-2.47 (m, 4H), 2.45 (s, 3H), 2.42-2.38 (m, 1H), 2.28 (s, 3H), 1.98-1.89 (m, 1H), 1.44-1.35 (m, 1H).

### Step 3: 1-methyl-3-((tritylthio)methyl)pyrrolidine (34-5)

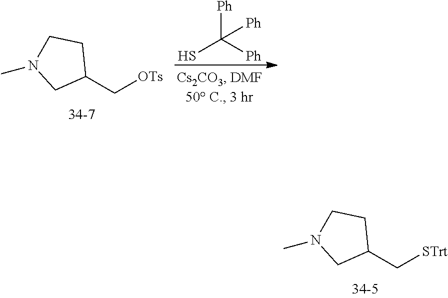

A mixture of (1-methylpyrrolidin-3-yl)methyl 4-methylbenzenesulfonate (12.7 g, 47.2 mmol, 1.00 eq), triphenylmethanethiol (15.6 g, 56.6 mmol, 1.20 eq), Cs₂CO₃ (23.0 g, 70.7 mmol, 1.50 eq), NaI (707 mg, 4.71 mmol, 0.10 eq) in DMF (90 mL) was degassed and purged with N₂ 3 times, and then the mixture was stirred at 50° C. for 3 hours under N₂. The reaction mixture was partitioned between EtOAc (500 mL×2) and water (500 mL×3). The organic phase was separated, washed with brine (500 mL), dried over anhydrous sodium sulfate, filtered and concentrated under reduced pressure to give a residue. The residue was purified by column chromatography (SiO₂, PE/EtOAc=3/1 to CH₂Cl₂/MeOH=10/1) to give compound 34-5 (16.9 g, 37.1 mmol, 81.5% yield, 82.0% purity) as a yellow oil.

¹H NMR (400 MHz, CDCl₃) δ=7.40-7.37 (m, 6H), 7.27-7.22 (m, 6H), 7.21-7.16 (m, 3H), 2.68-2.64 (m, 1H), 2.54-2.48 (m, 1H), 2.39-2.33 (m, 1H), 2.26 (s, 3H), 2.22-2.12 (m, 3H), 2.08-2.04 (m, 1H), 1.97-1.89 (m, 1H), 1.39-1.31 (m, 1H).

### Step 4: (1-methylpyrrolidin-3-yl)methanethiol (34-6)

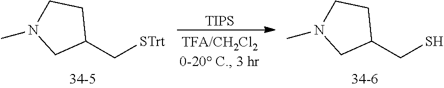

A mixture of 1-methyl-3-(tritylsulfanylmethyl)pyrrolidine (8.00 g, 21.4 mmol, 1.00 eq) in TFA (27 mL) and CH₂Cl₂ (80 mL), the mixture was degassed and purged with N₂ 3 times, then triisopropylsilane (6.78 g, 42.8 mmol, 8.80 mL, 2.00 eq) was added slowly at 0° C., and then the mixture was stirred at 20° C. for 3 hours under N₂. The reaction mixture was concentrated under reduced pressure. The filtrate was diluted with MeOH (20 mL) and extracted with PE (30 mL×5). The MeOH layers was concentrated under reduced pressure to give compound 34-6 (5.00 g, crude, TFA salt) as a light yellow oil.

¹H NMR (400 MHz, CDCl₃) δ=3.95-3.91 (m, 1H), 3.82-3.69 (m, 1H), 3.15-2.99 (m, 2H), 2.93 (s, 3H), 2.76-2.64 (m, 4H), 2.39-2.31 (m, 1H), 1.97-1.88 (m, 1H)

### Step 5: di(pentadecan-8-yl) 4,4′-(((((1-methylpyrrolidin-3-yl)methyl)thio)carbonyl)azanediyl)dibutanoate (CAT34)

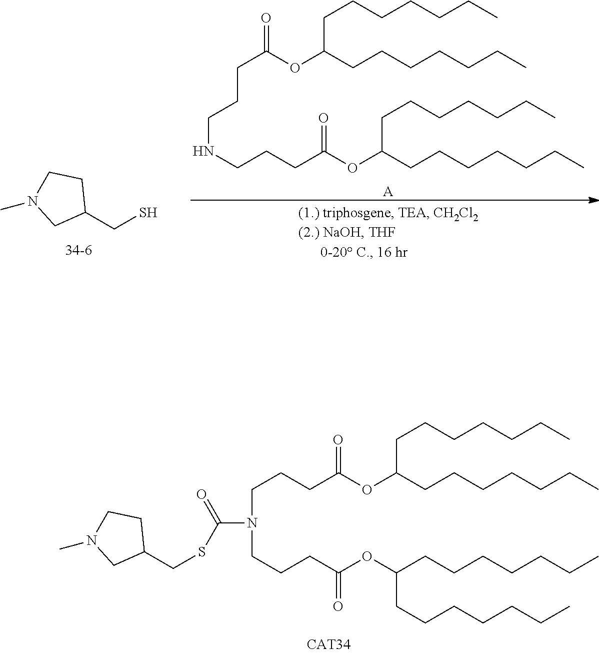

To a solution of 1-heptyloctyl 4-[[4-(1-heptyloctoxy)-4-oxo-butyl]amino]butanoate (2.50 g, 4.10 mmol, 1.00 eq) dissolved in dry CH₂Cl₂ (30 mL) were added TEA (1.24 g, 12.3 mmol, 1.71 mL, 3.00 eq) and triphosgene (1.09 g, 3.69 mmol, 0.90 eq) at 0° C. under N₂. The resulting solution was stirred at 20° C. for 1 hour. The resulting reaction was concentrated under reduced pressure. To a solution of (1-methylpyrrolidin-3-yl)methanethiol (3.02 g, 12.30 mmol, 3 eq, TFA salt) in dry THF (35 mL) was added NaOH (2.46 g, 61.48 mmol, 15 eq) at 0° C. under N₂. To this resulting solution was added carbamoyl chloride in THF (35.0 mL) at 0° C. under N₂. The resulting solution was stirred at 20° C. for 15 hours. The reaction mixture was quenched by NH₄Cl (100 mL) at 0° C. and then diluted with EtOAc (100 mL). The aqueous phase was extracted with EtOAc (100 mL×3). The combined organic phase was washed with brine (200 mL), dried with anhydrous sodium sulfate, filtered and concentrated in vacuum to give a residue. The residue was purified by flash silica gel chromatography (ISCO®; 120 g SepaFlash® Silica Flash Column, Eluent of 0˜37% EtOAc/PE gradient @ 100 mL/min), then was purified by prep-HPLC (HCl condition; column: Welch Ultimate C_{18 150*25} mm*5 um; mobile phase: [water (HCl) -MeOH]; B %: 70%-100%, 10 min) to give compound CAT34 (179 mg, 0.227 mmol, 5.8% yield, 97.2% purity) as a yellow oil.

### LCMS [M+1]⁺: 767.6

¹H NMR (400 MHz, CDCl₃) δ=4.90-4.84 (m, 2H), 3.26-3.48 (m, 4H), 3.03-2.92 (m, 2H), 2.78-2.74 (m, 1H), 2.60-2.51 (m, 2H), 2.50-2.44 (m, 1H), 2.35 (s, 3H), 2.32-2.25 (m, 4H), 2.12-2.03 (m, 1H), 1.96-1.84 (m, 4H), 1.78-1.67 (m, 2H), 1.48-1.55 (m, 8H), 1.32-1.22 (m, 40H), 0.88 (t, J=6.8 Hz, 12H).

### Example 1.35: Synthesis of CAT35

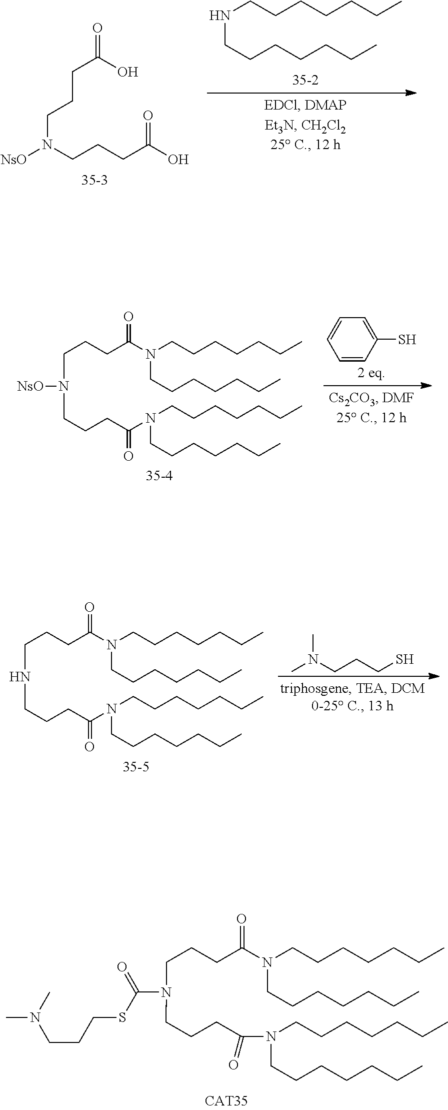

### Step 1: Synthesis of intermediate 2 (N-heptylheptan-1-amine) (35-2)

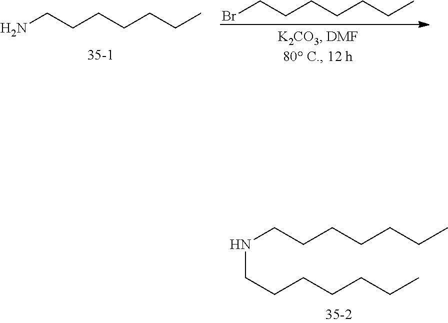

To a solution of heptan-1-amine (30 g, 260.38 mmol, 38.81 mL, 1 eq) and 1-bromoheptane (46.63 g, 260.38 mmol, 40.91 mL, 1 eq) in DMF (100 mL) was added K₂C₀₃ (35.99 g, 260.38 mmol, 1 eq). The mixture was stirred at 80° C. for 12 hr under N₂. The reaction mixture was quenched by the addition of water (500 mL), and then extracted with ethyl acetate (500 mL×3). The combined organic layers were washed with brine (200 mL), dried over sodium sulfate, filtered and concentrated under reduced pressure. The residue was purified by silica gel chromatography (Petroleum ether/Ethyl acetate=10/1 to 1/1) to give compound 35-2 (15 g, 70.29 mmol, 27.00% yield) as a yellow oil.

¹H NMR (400 MHz, CDCl₃) δ=2.38-2.34 (m, 4H), 1.48-1.42 (m, 4H), 1.38-1.22 (m, 16H), 0.90-0.84 (m, 6H).

### Step 2: 4-[[4-(diheptylamino)-4-oxo-butyl]-(4-nitrophenyl)sulfonyl-amino]-N,N-diheptyl-butanamide (35-4)

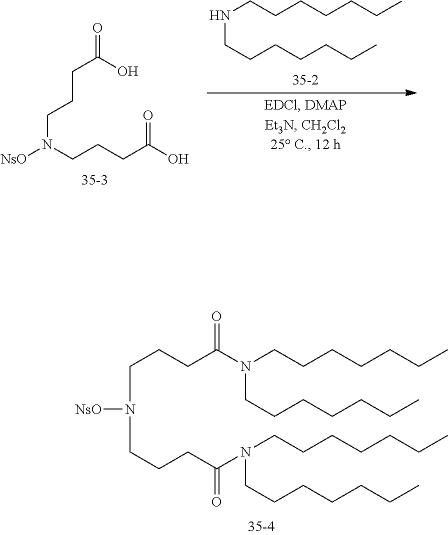

To a solution of 4-[3-carboxypropyl-(4-nitrophenyl)sulfonyl-amino]butanoic acid (3 g, 8.01 mmol, 1 eq) in dichlormethane (30 mL) was added EDCI (4.61 g, 24.04 mmol, 3 eq), then DMAP (489.50 mg, 4.01 mmol, 0.5 eq) and TEA (2.43 g, 24.04 mmol, 3.35 mL, 3 eq) were added. After 30 minutes, the N-heptylheptan-1-amine (3.59 g, 16.83 mmol, 2.1 eq) was added. Then, the mixture was stirred at 25° C. for 12 hr. The reaction mixture was quenched by the addition of water (100 mL), and then extracted with ethyl acetate (200 mL×3). The combined organic layers were washed with brine (100 mL), dried over sodium sulfate, filtered and concentrated under reduced pressure. The residue was purified by silica gel chromatography (Petroleum ether/Ethyl acetate=30/1 to 1/1) to give compound 35-4 (2.4 g, 3.14 mmol, 39.14% yield) as a yellow oil.

¹H NMR (400 MHz, CDCl₃) (5 8.27-8.24 (m, 2H), 8.08-7.80 (m, 2H), 3.23-3.17 (m, 4H), 3.12-3.06 (m, 3H), 2.58-2.52 (m, 1H), 2.27-2.18 (m, 3H), 1.84-1.48 (m, 4H), 1.53-1.28 (m, 8H), 1.20-1.05 (m, 32H), 0.88-0.75 (m, 12H).

### Step 3: 4-[[4-(diheptylamino)-4-oxo-butyl]amino]-N,N-diheptyl-butanamide (35-5)

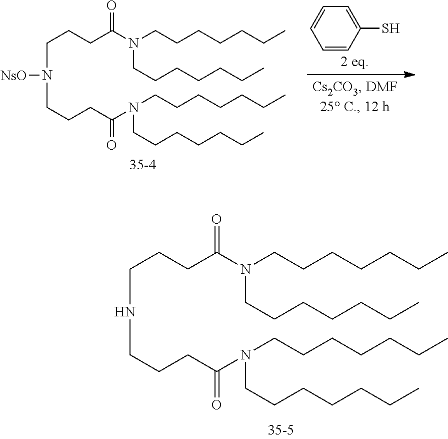

To a solution of 4-[[4-(diheptylamino)-4-oxo-butyl]-(4-nitrophenyl)sulfonyl-amino]-N,N-diheptyl-butanamide (1.8 g, 2.35 mmol, 1 eq) and benzenethiol (518.38 mg, 4.71 mmol, 479.99 uL, 2 eq) in DMF (20 mL) was added Cs₂CO₃ (1.53 g, 4.71 mmol, 2 eq). The mixture was stirred at 25° C. for 12 hr under N₂. The reaction mixture was quenched by the addition of water (100 mL), and then extracted with ethyl acetate (300 mL×3). The combined organic layers were washed with brine (500 mL), dried over sodium sulfate, filtered and concentrated under reduced pressure. The residue was purified by silica gel chromatography (Petroleum ether/Ethyl acetate=10/1 to 1/1 and Dichloromethane/Methanol=30/1 to 5/1) to give compound 35-5 (1 g, 1.72 mmol, 73.29% yield) as a yellow oil.

¹H NMR (400 MHz, CDCl₃) δ=3.32-3.21 (m, 8H), 2.80-2.68 (m, 4H), 2.48-2.25 (m, 4H), 1.96-1.81 (m, 4H), 1.56-1.46 (m, 8H), 1.28-1.10 (m, 32H), 0.93-0.85 (m, 12H).

### Step 4: S-[3-(dimethylamino)propyl] N,N-bis[4-(diheptylamino)-4-oxo-butyl]carbamothinate (CAT35)

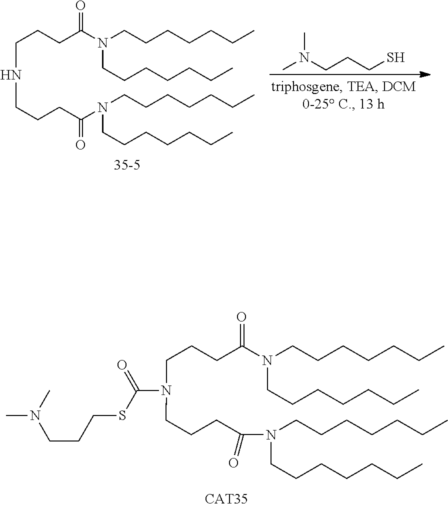

To a solution of 4-[[4-(diheptylamino)-4-oxo-butyl]amino]-N,N-diheptyl-butanamide (1.5 g, 2.59 mmol, 1 eq) dissolved in dry dichlormethane (20 mL) were added TEA (785.11 mg, 7.76 mmol, 1.08 mL, 3 eq) and triphosgene (383.74 mg, 1.29 mmol, 0.5 eq) at 0° C. under nitrogen atmosphere. The resulting solution was stirred at 25° C. under nitrogen atmosphere for 1 hr. The reaction was concentrated under reduced pressure and kept under nitrogen atmosphere. NaOH (724.11 mg, 18.10 mmol, 7 eq) was dissolved in dry THF (50 mL) at 0° C. under nitrogen atmosphere, then 3-(dimethylamino)propane-1-thiol (1.54 g, 12.93 mmol, 5 eq) was added under nitrogen atmosphere. To this resulting solution, carbamoyl chloride dissolved in THF (10 mL) was added slowly under nitrogen atmosphere at 0° C. The mixture was stirred at 35° C. for 12 hr. The reaction mixture was quenched by the addition of water (100 mL), and then extracted with ethyl acetate (200 mL×3). The combined organic layers were washed with brine (100 mL), dried over sodium sulfate, filtered and concentrated under reduced pressure. The residue was purified by silica gel chromatography (Petroleum ether/Ethyl acetate=10/1 to 1/1 and Dichloromethane/Methanol=30/1 to 5/1) and MPLC (column: Welch Ultimate XB-SiOH 250*50*10 um; mobile phase: [Hexane-EtOH]; B %: 0%-28%, 15 min) to give compound CAT35 (181 mg, 247.84 umol, 17.97% yield, 99.3% purity) as a yellow oil.

### LCMS: [M+H]⁺: 725.6

¹H NMR (400 MHz, CDCl₃) δ=3.48-3.41 (m, 4H), 3.28-3.20 (m, 4H), 3.18-3.10 (m, 4H), 2.50-2.10 (m, 4H), 1.96-1.60 (m, 6H), 1.53-1.46 (m, 8H), 1.26-1.10 (m, 32H), 0.95-0.81 (m, 12H).

### Example 1.36: Synthesis of CAT2

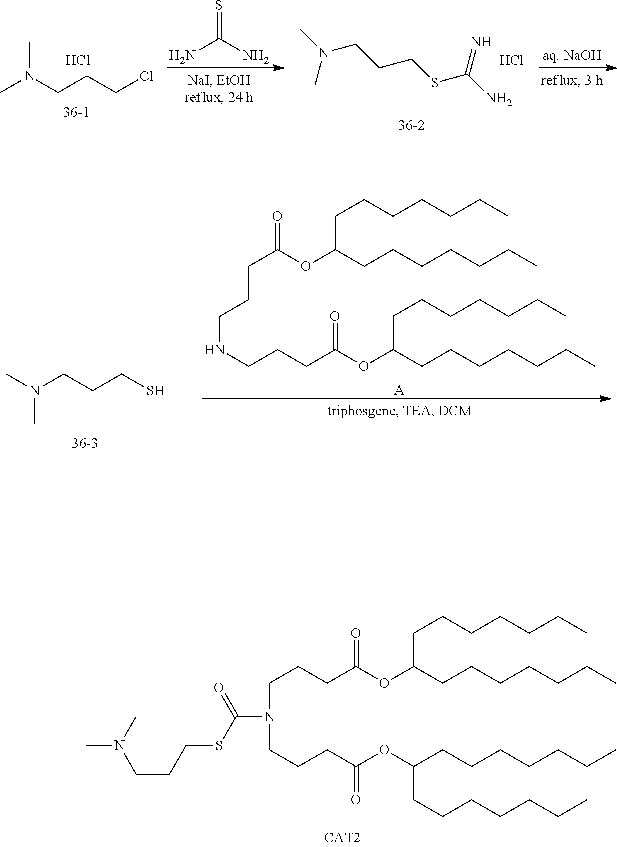

### Step 1: 2-[3-(dimethylamino)propyl]isothiourea hydrochloride (36-2)

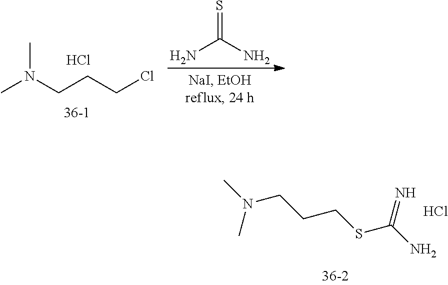

To a solution of 3-chloro-N,N-dimethyl-propan-1-amine (25 g, 158.16 mmol, 1 eq, HCl) in EtOH (500 mL) were added NaI (474.13 mg, 3.16 mmol, 0.02 eq) and thiourea (13.24 g, 173.97 mmol, 1.1 eq). The mixture was stirred at 80° C. for 16 hr. TLC (dichloromethane: methanol=10:1, PMA) indicated the starting material was consumed completely and one new main spot formed. The reaction mixture was cooled to 10° C. and crystal precipitation. The reaction mixture was filtered and the filter cake were washed with ethyl acetate (100 mL×2). The filter cake was concentrated in vacuum to give compound 36-2 (29.1 g, 147.17 mmol, HCl) as a white solid. The crude product was used for next step without further purification.

¹H NMR (400 MHz, CDCl₃) δ: 9.40-9.37 (m, 4H), 3.35 (t, J=7.6 Hz, 2H), 3.12 (t, J=7.6 Hz, 2H), 2.72 (s, 6H), 2.08-2.01 (m, 2H).

### Step 2: 3-(dimethylamino)propane-1-thiol (36-3)

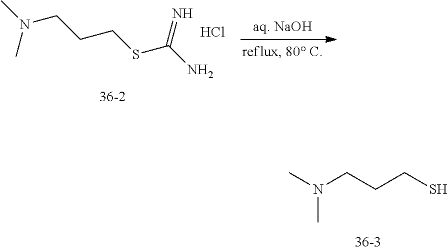

To a solution of 2-[3-(dimethylamino)propyl]isothiourea (10.0 g, 62.0 mmol, 1 eq) in H₂O (10 mL) and EtOH (40 mL) was added NaOH (14.9 g, 372 mmol, 6 eq). The mixture was stirred at 90° C. for 3 hours. The reaction mixture was cooled to 25° C., quenched by the addition of water (20 mL), and then extracted with ethyl acetate (20 mL×3). The combined organic layers were washed with brine (20 mL), dried over sodium sulfate, filtered and concentrated under reduced pressure to give compound 36-3 (2.10 g, crude) as a yellow oil. The reaction residue was used directly for the next step.

### Step 3: 1-heptyloctyl 4-[3-(dimethylamino)propylsulfanylcarbonyl-[4-(1-heptyloctoxy)-4-oxo-butyl]amino]butanoate (CAT2)

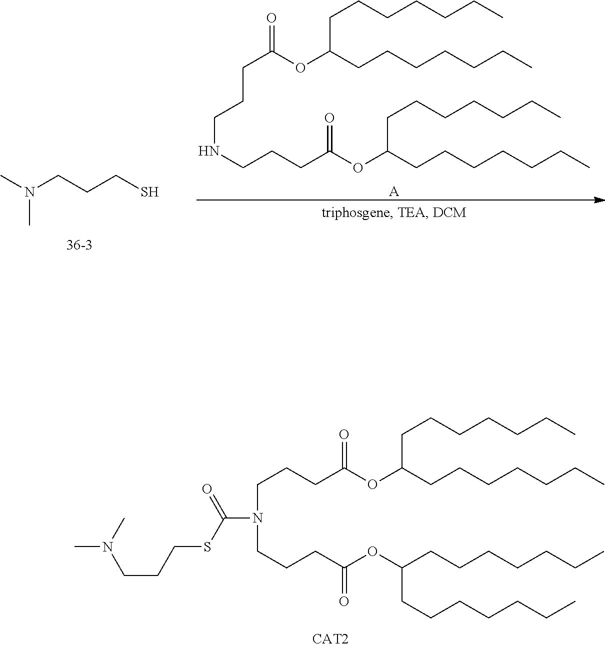

To a solution of 1-heptyloctyl 4-[[4-(1-heptyloctoxy)-4-oxo-butyl]amino]butanoate (2.00 g, 3.28 mmol, 1 eq) in DCM (20 mL) were added bis(trichloromethyl) carbonate (486 mg, 1.64 mmol, 0.5 eq) and TEA (995 mg, 9.84 mmol, 1.37 mL, 3 eq). After addition, the resulting solution was stirred at 20° C. for 1 hour. The resulting reaction was concentrated under reduced pressure. A solution of 3-(dimethylamino)propane-1-thiol (1.95 g, 16.4 mmol, 5 eq) in dry THF (20 mL) was added NaOH (918 mg, 23.0 mmol, 7 eq) at 0° C. under N₂. Carbamoyl chloride dissolved in THF (5 mL) was added at 0° C. under N₂. The resulting solution was stirred at 20° C. for 15 hours. The reaction mixture was quenched with saturated aqueous NH₄Cl (100 mL) and then diluted with ethyl acetate (100 mL). The aqueous phase was extracted with ethyl acetate (100 mL×3). The combined organic phase was washed with brine (100 mL), dried with anhydrous Na₂SO₄, filtered and concentrated in vacuum to give a residue. The residue was purified by column chromatography (SiO₂, petroleum ether: ethyl acetate=10:1 to 1:1) to afford CAT2 (260 mg, 0.340 mmol, 65% yield, 99% purity) was obtained as a yellow oil.

### LCMS: [M+H]⁺: 756.1;

¹H NMR (400 MHz, CDCl₃) δ: 4.82-4.77 (m, 2H), 3.39-3.29 (m, 4H), 2.84 (t, J=7.2 Hz, 2H), 2.31-2.22 (m, 6H), 2.17-2.15 (m, 6H), 1.85-1.70 (m, 6H), 1.46-1.42 (m, 8H), 1.25-1.10 (m, 40H), 0.86-0.72 (m, 12H).

### Example 2: Synthesis of PEG-Lipids

### Example 2.1: Synthesis of CHM-001

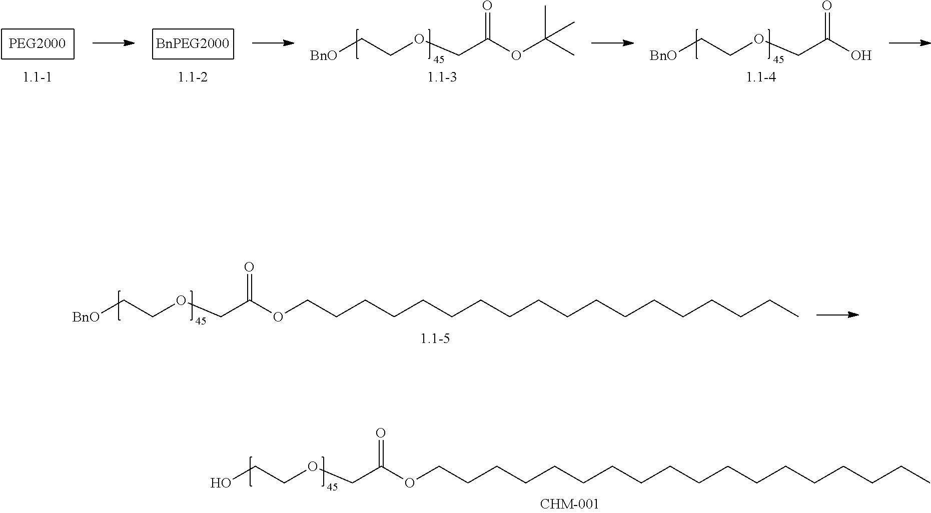

### Step 1: Synthesis of benzyl-poly(ethylene glycol)2000 (1.1-2)

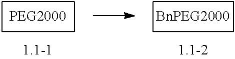

To a solution of PEG2000 (20 g, 10.00 mmol, 1 eq.) in THF (100 mL) at 0° C. was added NaH (599.83 mg, 15.00 mmol, 60% purity, 1.5 eq.), and stirred at 0° C. for 40 min. The reaction mixture was treated with bromomethyl benzene (2.57 g, 15.00 mmol, 1.78 mL, 1.5 eq.). The reaction mixture was then stirred at 25° C. for 18 h. The reaction mixture was quenched with saturated NH₄Cl solution (100 mL), and diluted with DCM (150 mL). The organic layer was washed with H₂O (70 mL×2) and brine (70 mL×2), dried over anhydrous Na₂SO₄. The resulting solution was concentrated at low pressure to afford the crude product as white solid. The crude product was purified by flash silica gel chromatography (0˜5%, MeOH/DCM) to afford the compound 1.1-2 (2.80 g, 1.34 mmol, 13.4% yield) as a white solid.

¹H-NMR (400 MHz, CHLOROFORM-d) δ 7.34-7.29 (m, 5H, PhCH₂—), 4.57 (s, 2H, PhCH₂-), 3.82-3.46 (m, 180H, poly (ethylene glycol) 2000).

### Step 2: Synthesis of tert-Butyl 2-(Benzyl-poly (ethylene glycol) 2000)-acetate (1.1-3)

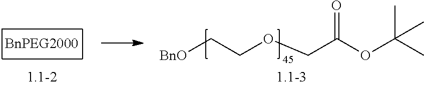

To a mixture of benzyl-poly(ethylene glycol)2000 (1.1-2, 2.8 g, 544.6 μmol, 1 eq.) in THF (25 mL) was added NaH (535.8 mg, 13.39 mmol, 60% purity, 10 eq.) in portions at 0° C. under N₂. The reaction mixture was stirred at 0° C. for 30 min, and tert-butyl 2-bromoacetate (1.83 g, 9.38 mmol, 1.39 mL, 7 eq.) was added to the above mixture. The reaction mixture was stirred at 26° C. for 18 h. The mixture was quenched with H₂O (20 mL) and diluted with DCM (50 mL). The organic layer was separated and the aqueous phase was extracted with DCM (20 mL×2). The combined organic phase was washed with brine (20 mL×2), dried with anhydrous Na₂SO₄, filtered and concentrated in vacuum to afford crude product as white solid. The crude product was purified by flash chromatography (0-5%, DCM/MeOH) to afford the compound 1.1-3 (3.94 g, 1.79 mmol, 74.7% yield) as a white wax-like solid. 1H NMR (400 MHz, CHLOROFORM-d) δ 7.43-7.30, 5H, PhCH₂-), 4.58(s, 2H, PhCH₂-), 4.03, 2H, —O—CH₂—CO₂-), 3.82-3.46 (m, 180H, poly(ethylene glycol)2000), 1.49 (s, 9H, ^{t}Bu).

### Step 3. Synthesis of 2-(benzyl-poly(ethylene glycol)2000)-acetic acid (1.1-4)

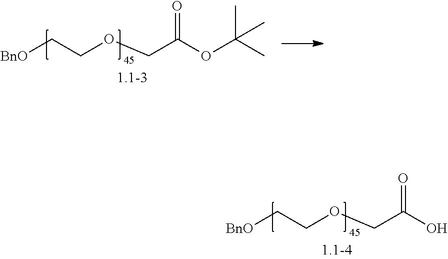

To a solution of tert-butyl-2-(benzyl-poly(ethylene glycol)2000)-acetate (1.20 g, 1.79 mmol, 1 eq.) in DCM (10 mL) was added TFA (7.70 g, 67.53 mmol, 5 mL, 37.79 eq.) in portions at 26° C. The mixture was stirred at 26° C. for 18 h. The mixture was concentered in vacuum to afford the crude product 1.1-4 (1.5 g, crude) as a yellow oil, which was directly used in the next step without further purification.

### Step 4. Synthesis of octadecyl 2-(benzyl-poly(ethylene glycol)2000)-acetate (1.1-5)

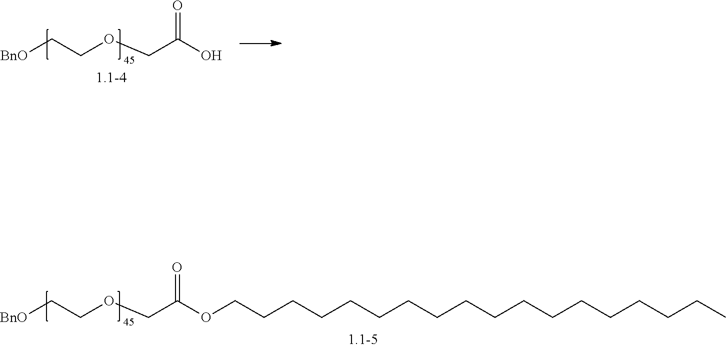

To a solution of 2-(benzyl-poly(ethylene glycol)2000)-acetic acid (1.17 g, crude), octadecan-1-ol (2.95 g, 10.89 mmol, 3.63 mL, 20 eq.) and DMAP (133.06 mg, 1.09 mmol, 2 eq.) in DCM (10 mL) was added EDCI (2.09 g, 10.89 mmol, 20 eq.) in one portion at 26° C. under N₂. The mixture was stirred at 26° C. for 18 hours. TLC (DCM/MeOH=10:1) indicated a new spot with slightly lower polarity was found. The mixture was quenched with H₂O (20 mL) and diluted with DCM (50 mL). The organic layer was separated and the aqueous phase was extracted with DCM (30 mL×2). The combined organic phase was washed with brine (30 mL×2), dried with anhydrous Na₂SO₄, filtered and concentrated in vacuum to afford the crude product as a white solid. The residue was purified by flash chromatography (0-5%, DCM/MeOH) to afford the desired product octadecyl 2-(Benzyl-poly(ethylene glycol)2000)—acetate (1.01 g, ˜76% yield) as a white wax-like solid.

¹H NMR (400 MHz, CHLOROFORM-d) δ 7.34-7.25 (m, 5H, PhCH₂—), 4.54 (s, 2H, PhCH₂—), 4.14-4.09 (4H, —O—CH₂—CO₂—CH₂—), 3.82-3.46 (m, 180H, poly(ethylene glycol)2000), 1.66-1.55 (m, 2H, Me(CH₂)₁₅—CH₂—), 1.23 (s, 22H, Me(CH₂)₁₅—), 0.85 (t, J=6.8 Hz, 3H, Me(CH₂)₁₅—).

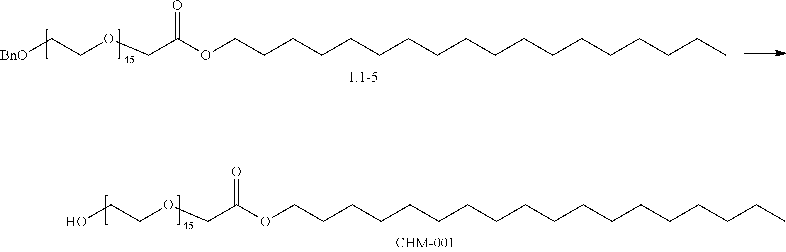

### Step 5. Synthesis of octadecyl 2-(poly(ethylene glycol)2000)-acetate (CHM-001)

To a solution of Octadecyl 2-(benzyl-poly(ethylene glycol)2000)-acetate (1.01 g, 420.66 μmol, 1 eq.) in EtOH (60 mL) was added Pd(OH)₂/C (3.01 g, 10% purity) at 26° C. under H2 (15 psi) atmosphere. The mixture was stirred at 26° C. for 18 h. The reaction mixture was filtered and the filtrate was concentrated at low pressure to afford the crude product as a white solid. The crude product was purified by flash silica gel chromatography (0˜6%, MeOH/DCM) to afford the desired product CHM-001 (0.29 g, 123.60 μmol, 29.38% yield) as a white wax-like solid.

¹H NMR (400 MHz, CHLOROFORM-d) δ 4.18-4.12 (m, 4H, —CH₂—(CO)O—CH₂-), 3.75-3.56 (m, 180H, polyethylene glycol 2000), 1.69-1.60 (m, 2H, —(CO)O—CH₂—CH₂-), 1.27 (s, 30H, Me-(CH₂)₁₅-), 0.89 (t, J=6.8 Hz, 3H, Me-). ¹³C NMR (400 MHz, CHLOROFORM-d) δ 170.59, 72.75, 70.89, 70.55, 70.21, 68.66, 65.00, 61.67, 31.93, 29.70, 29.66, 29.52, 29.37, 25.85, 22.69, 14.13. HPLC (ELSD), Rt=3.36 min, 98.49% purity. IR(vmax/cm), 3491, 2887, 1968, 1752, 1467, 1360, 1343, 1280, 1149, 1112, 963, 842, 720. Melting range, 50.7-51.7° C.

### Example 2.2: Synthesis of CHM-004

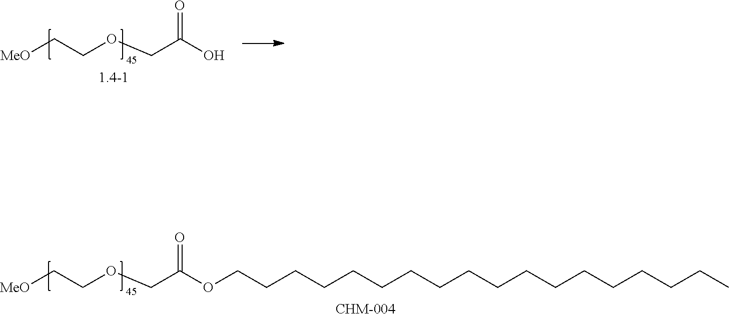

1.4-1 (500 mg, 241.26 μmol, 1 eq.) was dissolved in dry DCM (10 mL). DMAP (58.95 mg, 482.52 μmol, 2 eq.) and EDCI (277.50 mg, 1.45 mmol, 6 eq.) were then added successively, followed by addition of octadecan-1-ol (391.56 mg, 1.45 mmol, 482.21 μL, 6 eq.). The reaction mixture was then stirred at 25° C. for 18 h. The reaction mixture was then concentrated in vacuum to afford the crude product as a white solid. The crude product was purified by flash silica gel chromatography (0˜5% MeOH/DCM) to afford the desired product octadecyl 2-(methyl-poly(ethylene glycol)2000)-acetate as a wax-like solid (CHM-004, 430 mg, 76.6% yield).

¹H-NMR (400 MHz, CHLOROFORM-d) 7 4.19-4.09 (m, 4H, —O—CH₂—CO₂—CH₂—), 3.74-3.60 (m, 180H, poly(ethylene glycol)2000), 3.38 (s, 3H, MeO—), 1.68-1.58 (m, 2H, Me(CH₂)₁₅—CH₂—), 1.25 (s, 30H, Me(CH₂)₁₅—), 0.88 (t, J=6.8 Hz, 3H, Me(CH₂)₁₅—). ¹HPLC (ELSD), Rt=7.88, 99.93% purity. IR (□max/cm⁻¹), 3479, 2887, 1750, 1467, 1360, 1343, 1148, 1112, 963, 842. Melting range, 50.6-51.3° C.

### Example 2.3: Synthesis of CHM-005

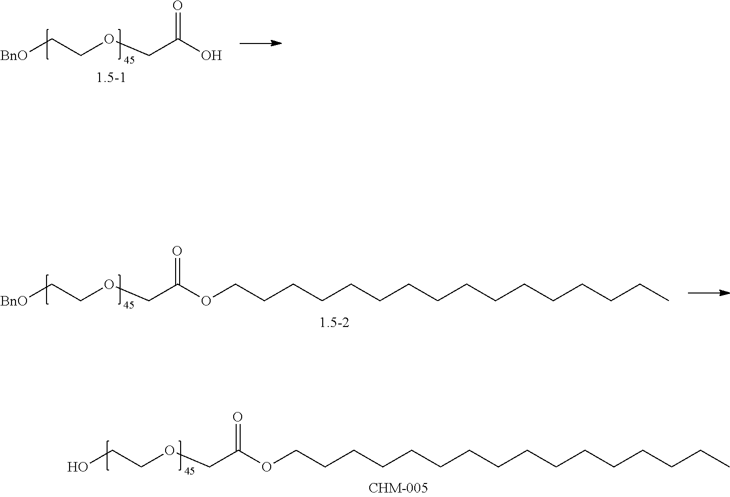

### Step 1: Synthesis of Hexadecyl 2-(benzyl-poly(ethylene glycol)2000)-acetate (1.5-2)

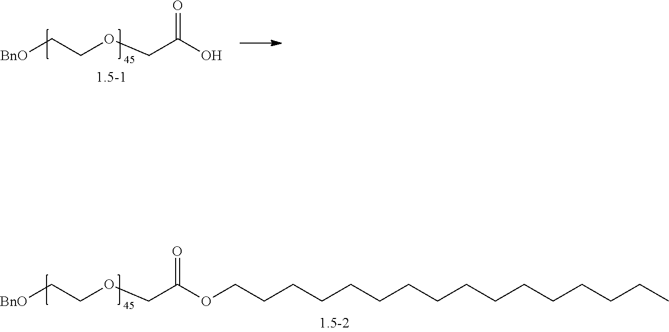

2-(benzyl-poly(ethylene glycol)2000)-acetic acid (1.5-1, 800 mg, 372.35 μmol, 1.00 eq.) was dissolved in DCM (10 mL), and DMAP (90.98 mg, 744.69 μmol, 2.00 eq.) and EDCI (713.80 mg, 3.72 mmol, 10 eq.) were added, followed by addition of hexadecan-1-ol (902.72 mg, 3.72 mmol, 10 eq.). The reaction mixture was stirred at 25° C. for 18 h. The reaction mixture was concentrated in vacuum to afford the crude product as a white solid. The crude product was purified by flash silica gel chromatography (0˜8%, MeOH/DCM) to afford compound 1.5-2 (110 mg, 38.01 μmol, 10.21% yield, 82% purity) as a white solid.

### Step 2. Synthesis of Hexadecyl 2-(poly(ethylene glycol)2000)-acetate (CHM-005)

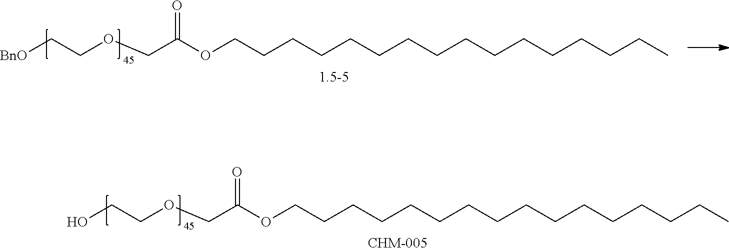

Hexadecyl 2-(benzyl-poly(ethylene glycol)2000)-acetate (1.5-2, 100 mg, 42.14 μmol, 1 eq.) was dissolved in EtOH (5 mL), and Pd(OH)₂ (50 mg, 71.21 μmol, 20% purity) was added. The reaction mixture was stirred at 20° C. under H2 atmosphere for 18 h. The reaction mixture was then filtered and the filtrate was concentrated at low pressure to afford the crude product as a white solid. The crude product was triturated with n-hexane at 20° C. for 30 min, filtered and the filter cake was collected and dried under reduced pressure to afford the compound CHM-005 (60 mg, 26.13 μmol, 61.99% yield, 99.4% purity) as a white solid.

¹H-NMR (400 MHz, CHLOROFORM-d) δ 4.19-4.10 (m, 4H, —CH₂—(CO)O—CH₂—), 3.77-3.57 (m, 180H, polyethylene glycol 2000), 1.70-1.59 (m, 2H, —(CO)O—CH₂—CH₂—), 1.26 (s, 26H, Me-(CH₂)₁₃—), 0.93-0.81 (m, 3H, Me-). HPLC (ELSD), Rt=5.93 min, 99.44% purity. IR(vmax/cm⁻¹), 3474, 2887, 1749, 1740, 1467, 1359, 1343, 1148, 1114, 963, 842. Melting range, 50.6-51.1° C.

### Example 2.4: Synthesis of CHM-006

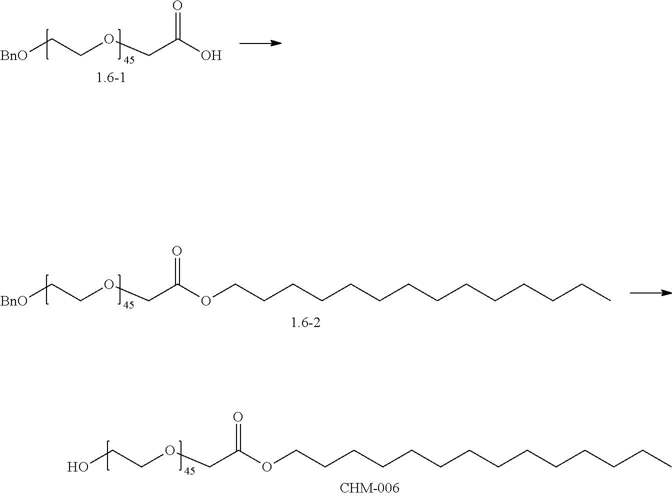

### Step 1: Synthesis of tetradecyl 2-(benzyl-poly(ethylene glycol)2000)-acetate (1.6-2)

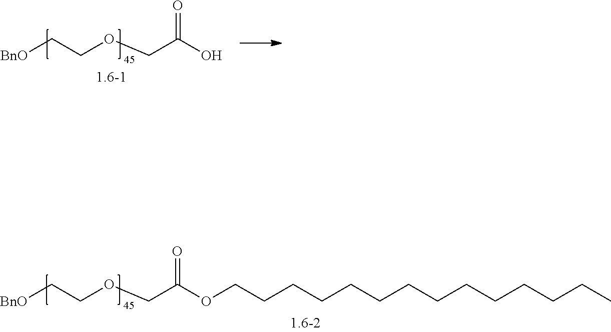

2-(benzyl-poly(ethylene glycol)2000)-acetic acid (1.6-1) (800 mg, 372.35 μmol, 1.00 eq.) was dissolved in DCM (10 mL), and DMAP (90.98 mg, 744.70 μmol, 2 eq.) and EDCI (713.80 mg, 3.72 mmol, 10 eq.) were then added, followed by addition of tetradecan-1-ol (798.26 mg, 3.72 mmol, 10 eq.). The reaction mixture was stirred at 20° C. for 18 h. The reaction mixture was then concentrated in vacuum to afford the crude product as a white solid. The crude product was purified by flash silica gel chromatography (0˜5%, MeOH/DCM) to afford the compound 1.6-2 (130 mg, 23.28 μmol, 14.9% yield) as a white solid.

### Step 2. Synthesis of tetradecyl 2-(poly(ethylene glycol)2000)-acetate (CHM-006)

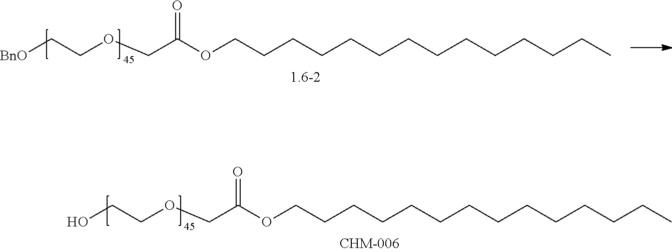

Tetradecyl 2-(benzyl-poly(ethylene glycol)2000)-acetate (1.6-2) (120 mg, 51.2 μmol, 1.00 eq.) was dissolved in EtOH (5 mL), and Pd(OH)₂ (100 mg, 10% purity) was then added. The reaction mixture was then stirred at 20° C. under H2 atmosphere for 18 h. The reaction mixture was then filtered and the filtrate was concentrated at low pressure to afford the crude product as a white solid. The crude product was triturated with n-hexane at 20° C. for 30 min. The solid was collected and dried under vacuum to afford compound CHM-006 (102 mg, 44.69 μmol, 87.33% yield, 98.79% purity) as a white solid.

¹H NMR (400 MHz, CHLOROFORM-d) δ 4.20-4.09 (m, 4H, —CH₂—(CO)O—CH₂—), 3.71-3.57 (m, 180H, polyethylene glycol 2000), 1.64 (q, J=6.8 Hz, 2H, —(CO)O—CH₂—CH₂—), 1.26 (s, 22H, Me—(CH₂)₁₁—), 0.92-0.81 (m, 3H, Me—). HPLC (ELSD), Rt=4.20 min, 98.79% purity. IR(Dₘₐₓ/cm⁻¹), 3447, 2889, 1749, 1740, 1653, 1466, 1358, 1343, 1148, 1113, 963, 843. Melting range, 49.7-50.1° C.

### Example 2.5: Synthesis of CHM-012

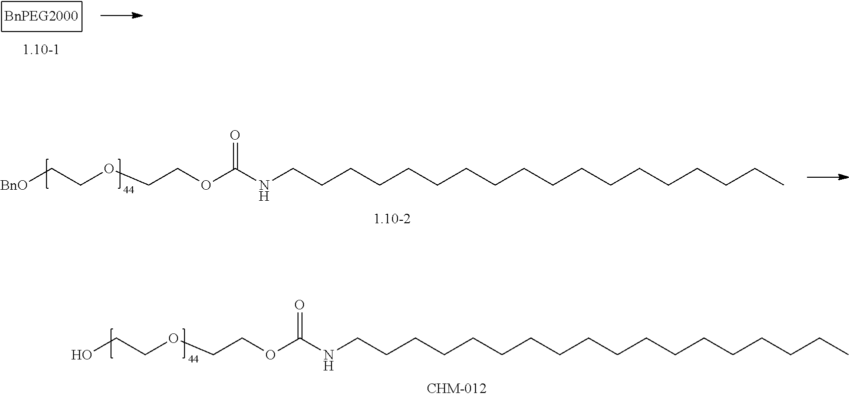

### Step 1: (Benzyl poly (ethylene glycol)2000)N-octadecylcarbamate (1.10-2)

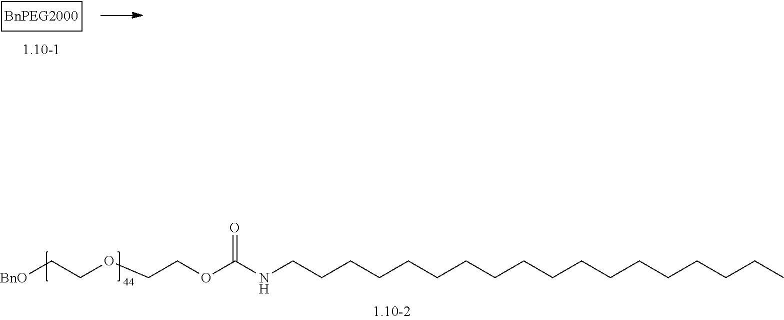

Benzyl-poly(ethylene glycol)2000 (BnPEG2000, 1.00 g, 685.26 μmol, 1.00 eq.) was dissolved in pyridine (10 mL), and 1-isocyanato heptadecane (1.93 g, 6.85 mmol, 10.0 eq) was then added. The reaction mixture was then refluxed at 80° C. for 18 h. The reaction mixture was then concentrated in vacuum to afford the crude product as a white solid. The crude product was purified by flash silica gel chromatography (0˜5%, MeOH/DCM) to afford compound 1.10-2 (850 mg, 326.94 μmol, 47.71% yield, 89% purity) as a white solid. ¹H-NMR (400 MHz, CHLOROFORM-d) δ 7.34 (d, J=4.3 Hz, 5H, PhCH₂—), 4.57 (s, 2H, PhCH₂—), 4.21 (br s, 2H, —CH₂—O(CO)—), 3.65 (s, 167H, poly(ethylene glycol)2000), 3.15 (br d, J=5.7 Hz, 2H, —CH₂—O(CO)NH—CH₂—), 1.48 (br s, 2H, —O(CO)NH—CH₂—CH₂—), 1.26 (s, 30H, Me(CH₂)₁₅—), 0.88 (br s, 3H, Me(CH₂)₁₅—).

### Step 2. Poly(ethylene glycol)2000 N-octadecylcarbamate (CHM-012)

(Benzyl-poly(ethylene glycol)2000)N-octadecylcarbamate (490 mg, 206.6 μmol, 1.00 eq.) was dissolved in EtOH (10 mL), and Pd(OH)₂/C (20 mg, 10% purity) was then added. The reaction mixture was then stirred at 20° C. under H2 atmosphere for 18 h. The reaction mixture was filtered and the filtrate was concentrated in vacuum to afford the crude product as a white solid. The crude product was purified by reversed-phase HPLC (column: Boston Prime C_{18 150*30} mm*5 um; mobile phase: [H₂O-MeOH]; B %: 60%-95%, 9 min) to afford compound CHM-012 (144 mg, 62.61 μmol, 30.31% yield, 99.82% purity) as a white solid. ¹H-NMR (400 MHz, CHLOROFORM-d) δ 4.22 (br s, 2H, —CH₂—O(CO)—), 3.65 (s, 180H, poly(ethylene glycol)2000), 3.16 (q, J=6.5 Hz, 2H, —CH₂—O(CO)NH—CH₂—), 2.77 (br s, 1H, HO— or —NH—), 1.48 (br s, 2H, —O(CO)NH—CH₂—CH₂—), 1.26 (s, 30H, Me(CH₂)₁₅—), 0.90-0.87 (m, 3H, Me(CH₂)₁₅—). HPLC (ELSD), Rt=6.40, 99.82% purity. IR (νₘₐₓ/cm⁻¹): 3307, 2916, 2887, 1963, 1694, 1548, 1467, 1360, 1344, 1149, 1113, 963, 842. Melting range, 45.5-46.3° C.

### Example 3: BRIJ™ S100 Stabilizes Both the Size and Encapsulation of Lipid Nanoparticles after a Freeze/Thaw Cycle at −20° C.

LNPs in this example comprise a lipid composition of SS—OC: Chol: DSPC: PEG2k-DPG at 49: 28.5: 22: 0.5 mol %, and encapsulate the RNA molecule encoding wild-type Seneca Valley virus (SVV) at a lipid-nitrogen-to-phosphate ratio (N:P) of 14. Total lipid concentration was set to 40 mM. RNA acidifying buffer was malic acid pH 3. LNPs were dialyzed overnight into the appropriate buffer (25 mM Tris, 50 mM sucrose, 113 mM NaCl, pH 7.4) and passed through a 0.2 μm filter after dialysis. Each of the cryo-protectants (propylene glycol (PG), BRIJ™ S100 (polyethylene glycol), Tween 80 (T80)) were spiked into LNPs during dilution to the various concentrations. Three concentrations of each cryo-protectant were examined as compared to a no excipient control.

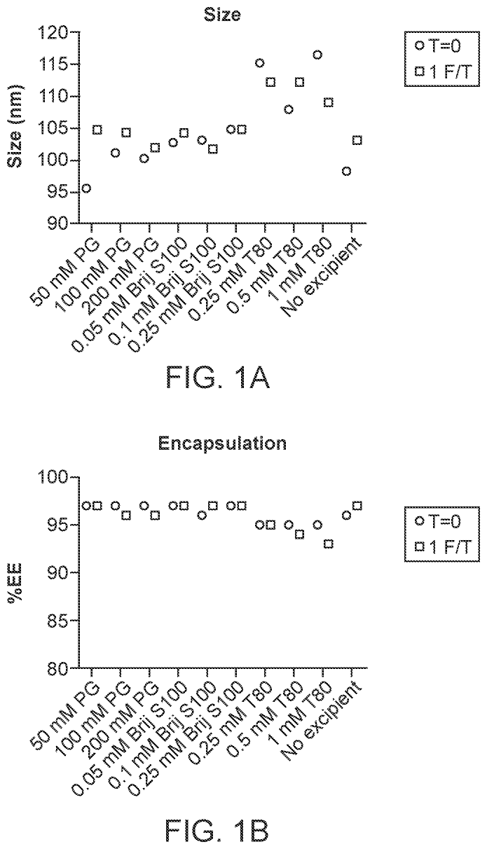

For freeze/thaw experiments, 0.5 mL vial was filled with LNPs at 0.5 mg/mL RNA concentration in 2 mL glass vials. The vials were subject to freezing at −20° C. overnight, then quickly thawed in 25° C. water bath. Time 0 characterization was executed on all samples. 0.5 mL sample volumes were frozen at −20° C. for at least 18 hours and subsequently thawed in a 25° C. water bath for 30 minutes. Upon complete thaw, vials were inverted to mix and post-1 freeze/thaw (F/T) characterization was executed. Size was measured by dynamic light scattering (DLS) (FIG. 1A) and encapsulation efficiency was measured by a fluorescence-based solution assay using RiboGreen RNA quantitation reagent (FIG. 1B).

Among these conditions, addition of 0.25 mM Brij S100 to the buffer (25 mM Tris, 50 mM sucrose, 113 mM NaCl, pH 7.4) worked best at maintaining both particle size and encapsulation after a single freeze/thaw cycle at −20° C.

### Example 4: Comparison of PEG2k-DPG, PEG2k-DMG and BRIJ™ S100 as PEG-lipid Component in LNP Formulation

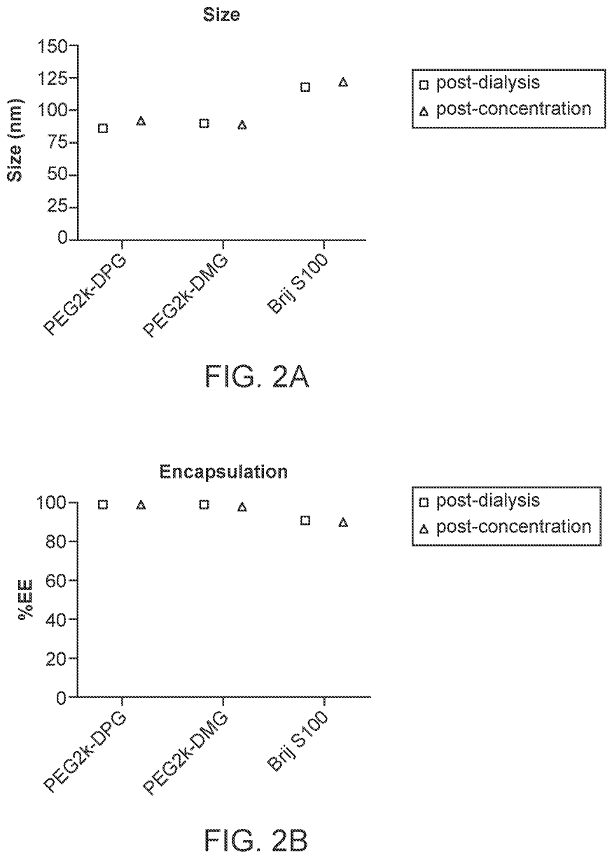

SS—OC: Cholesterol:DSPC: PEG-lipid (49:28.5:22:0.5 mol %) LNPs encapsulating non-replicating SVV RNA (SVV-neg) were prepared following similar procedures as in Example 1. The PEG-lipid was PEG2k-DPG, PEG2k-DMG or Brij S100. The N:P ratio was set to 14. Total lipid concentration was set to 40 mM. Formulations were mixed at a 3:1 aqueous:organic volume ratio at 12 mL/min with 60° C. heat applied to the organic phase syringe. Formulations were dialyzed against 1X PBS pH 7.2 for at least 18 hours. Characterization was executed post-dialysis. Formulations were concentrated using 100kD Amicon centrifugal filtration units. Characterization was executed post-concentration and compared to post-dialysis characterization. Size was measured by dynamic light scattering (FIG. 2A) and encapsulation efficiency was measured by RiboGreen (FIG. 2B).

The results showed that Brij S100 could be used in replacement of PEG2k-DPG or PEG2k-DMG for LNP formulation. In this particular example, the particle size was larger for LNPs comprising Brij S100.

### Example 5: LNPs Comprising Brij Displayed Altered Pharmacokinetic Characteristics in vivo upon Repeat Dosing

SVV-neg/SS—OC:Cholesterol:DSPC:PEG-lipid (49:28.5:22:0.5 mol %) LNPs were prepared according to Table 4 below. The PEG-lipid was either PEG2k-DPG or Brij S100.

TABLE 4

| Formulation parameters | | | | | | | | | |
|---|---|---|---|---|---|---|---|---|---|
| | | | Input | | | | | | |
| | | Lipid | RNA | | | Characterization | | | |
| | SS-OC:Cholesterol:DSPC:PEG- | conc. | conc. | | Flow | Size | | [RNA] | |
| PEG-lipid | lipid (mol %) | (mM) | (mg/mL) | N:P | ratio | (nm) | PDI | (μg/mL) | % EE |
| PEG2k- | 49:28.5:22:0.5 | 40 | 0.298 | 14 | 3 | 94 | 0.12 | 380 | 92 |
| DPG | | | | | | | | | |
| Brij S100 | 49:28.5:22:0.5 | 40 | 0.298 | 14 | 3 | 132 | 0.11 | 235 | 88 |

PDI: polydispersity index; % EE: Encapsulation Efficiency.

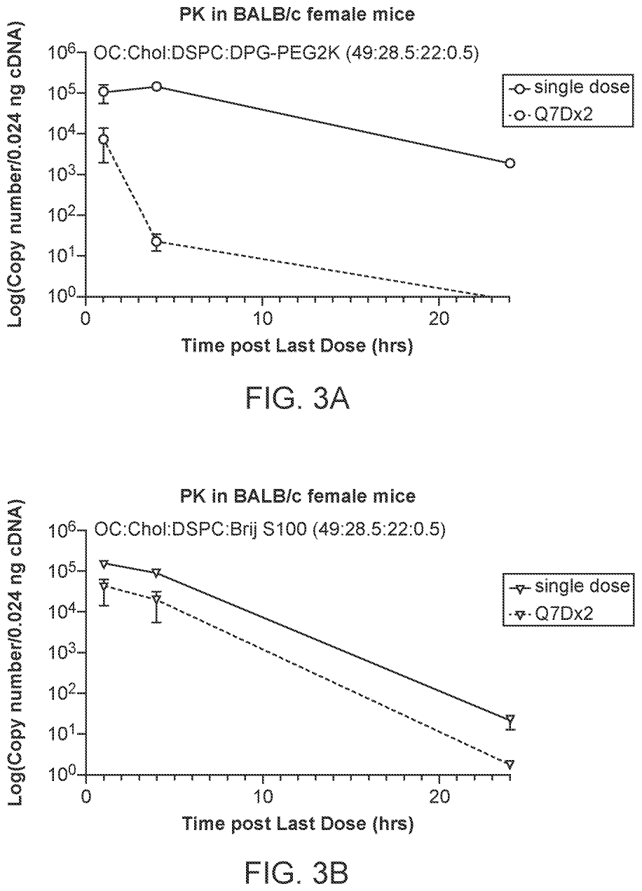

Formulations were used in a repeat dose (weekly dose schedule for 2 weeks, Q7×2) intravenous (IV) mouse PK study. Copy number of RNA in serum post-dose was measured at each time point. The results are shown in FIG. 3A (for PEG2k-DPG) and FIG. 3B (for Brij S100).

LNP comprising PEG2k-DPG exhibited prolonged circulation post-first dose with rapid clearance within 4 hours upon the second dose. LNP comprising Brij 5100 exhibited an intermediate change in exposure post-first dose but maintained similar circulation characteristic and slopes of elimination upon the second dose.

### Example 6: Lower Lipid Concentration and Changing RNA Buffer Reduce Size and Increase Encapsulation Efficiency of LNPs Formulated with Brij Molecules

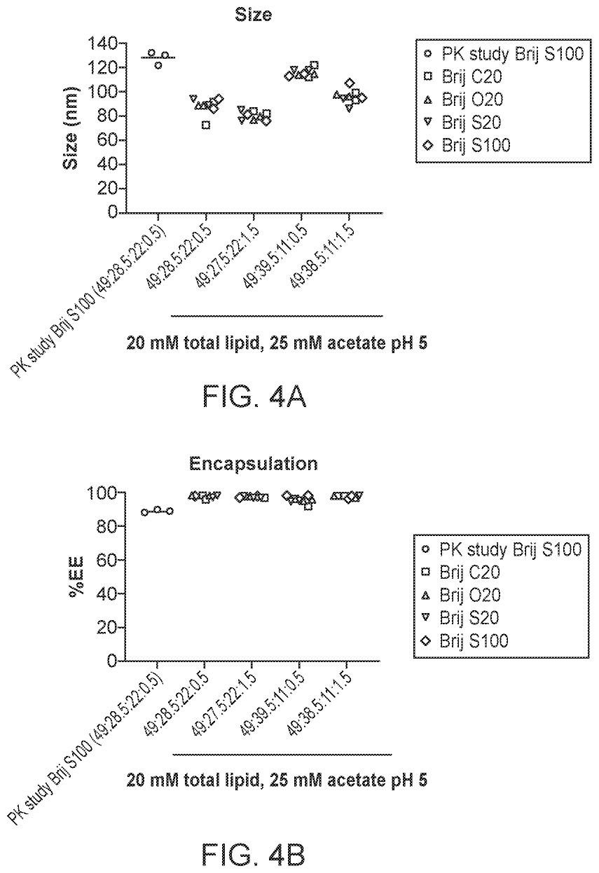

LNPs comprising SVV-neg/SS—OC:Cholesterol:DSPC:Brij were prepared at four different lipid mol % ratios: 49:28.5:22:0.5, 49:27.5:22:1.5, 49:39.5:11:0.5, and 49:38.5:11:1.5. The Brij molecule was Brij C₂₀, Brij 020, Brij S20 or Brij S100. The N:P ratio was set to 14 noting 2 ionizable amines in SS—OC. LNP preparation followed similar procedures as those in the previous examples. However, total lipid concentration was changed from 40 mM to 20 mM, and the RNA acidifying buffer was changed from 20 mM malic acid pH 3 to 25 mM acetate pH 5. Formulations were mixed at a 3:1 aqueous:organic volume ratio at 12 mL/min without any heat during mixing. Formulations were dialyzed against 1X PBS pH 7.2 for at least 18 hours. Formulations were concentrated using 100kD Amicon centrifugal filtration units. Characterization was executed. Size was measured by dynamic light scattering (FIG. 4A) and encapsulation efficiency was measured by RiboGreen (FIG. 4B). Each unique composition was formulated at least two times on separate days to ensure reproducibility.

The results showed that reducing the lipid concentration and changing the RNA acidifying buffer collectively resulted in smaller particle size and higher encapsulation across all Brij molecules and at each molar composition as compared to the previous OC/Brij S100 formulation (40 mM total lipid, 20 mM malic acid pH 3) that was used in Example 3 for the repeat dose mouse PK study.

### Example 7: LNPs Comprising Brij and Oncolytic Viral RNA Demonstrate High Anti-Tumor Efficacy in Animal Models

SVV-wt/SS—OC:Cholesterol:DSPC:PEG-lipid LNPs were prepared and characterized according to Table 5 below. The PEG-lipid was PEG2k-DPG, Brij S100, Brij C20 or Brij S20. PDI: polydispersity index; % EE: Encapsulation Efficiency; ZP: zeta potential.

TABLE 5

| | SS-OC:Chol:DSPC:PEG- | | Size | | ZP | |
|---|---|---|---|---|---|---|
| PEG-Lipid | lipid | Buffer | (nm) | PDI | (mV) | % EE |
| PEG2k-DPG | 49:28.5:22:0.5 | 20 mM malic acid | 88.7 | 0.1 | −3.7 | 100% |
| | | pH 3.0 | | | | |
| Brij S100 | 49:28.5:22:0.5 | 25 mM acetate | 107.8 | 0.12 | −0.6 | 96% |
| | | pH 5.0 | | | | |
| Brij S100 | 49:27.5:22:1.5 | 25 mM acetate | 91.8 | 0.14 | −2.0 | 93% |
| | | pH 5.0 | | | | |
| Brij S100 | 49:38.5:11:1.5 | 25 mM acetate | 81.4 | 0.14 | −0.9 | 98% |
| | | pH 5.0 | | | | |
| Brij C20 | 49:28.5:22:0.5 | 25 mM acetate | 108.7 | 0.14 | −8.8 | 98% |
| | | pH 5.0 | | | | |
| Brij S20 | 49:28.5:22:0.5 | 25 mM acetate | 100.6 | 0.11 | −6.6 | 99% |
| | | pH 5.0 | | | | |

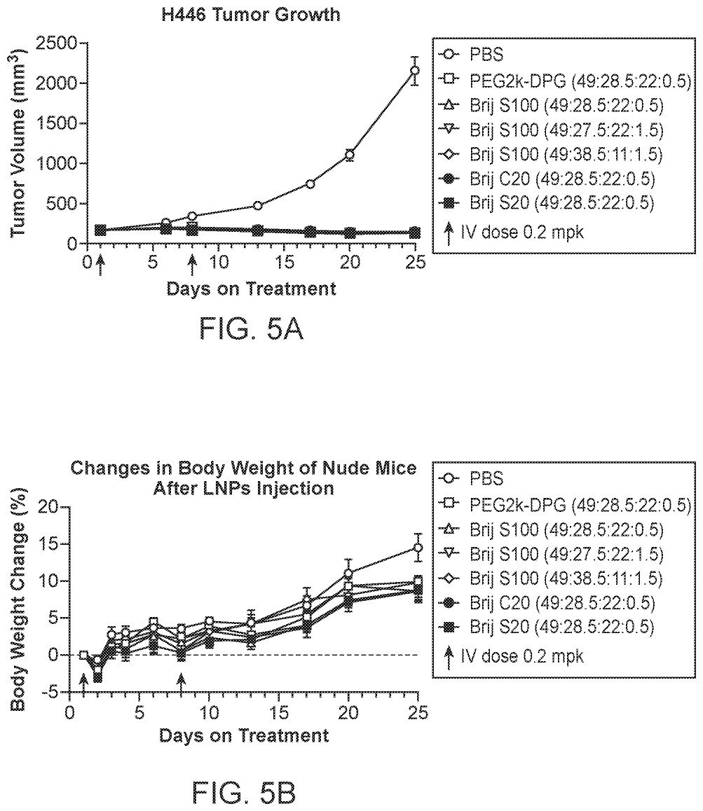

Formulations were used in a repeat dose IV mouse efficacy screen in H446 tumor model. Tumor volume (FIG. 5A) and body weight (FIG. 5B) were measured at each time point. The results showed that all formulations demonstrated high anti-tumor efficacy and were well tolerated. SS—OC/Brij LNPs were similar in efficacy and tolerability as compared to SS—OC/PEG2k-DPG.

In another study, SVV-wt/Ionizable lipid:Cholesterol: DSPC: Brij S100 (49:28.5:22:0.5 or 49:38.5:11:1.5 mol %) LNPs were prepared according to Table 6 below. The ionizable lipid was COATSOME® SS—OC or COATSOME® SS—OP.

TABLE 6

| | | Inoizable | | | |
|---|---|---|---|---|---|
| Ionizable | PEG- | Lipid:Chol:DSPC:Brij | Size | | % |
| Lipid | Lipid | S100 (mol %) | (nm) | PDI | EE |
| SS-OC | Brij S100 | 49:28.5:22:0.5 | 118.4 | 0.11 | 95% |
| SS-OP | Brij S100 | 49:28.5:22:0.5 | 146.2 | 0.11 | 91% |
| SS-OP | Brij S100 | 49:38.5:11:1.5 | 157.9 | 0.13 | 93% |

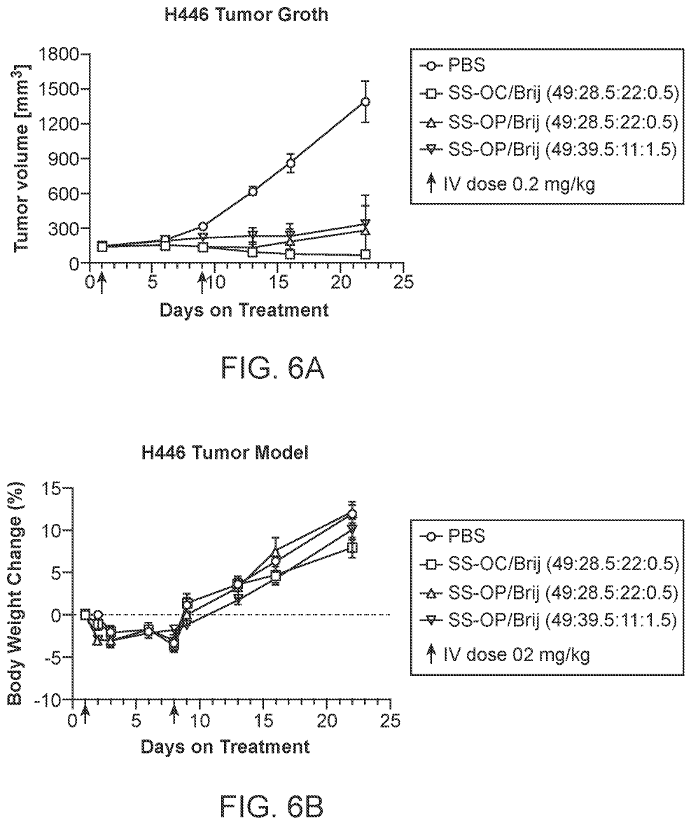

Formulations were used in a repeat dose IV mouse efficacy screen in H446 tumor model. Tumor volume (FIG. 6A) and body weight (FIG. 6B) was measured at each time point. The results showed that all formulations demonstrated high anti-tumor efficacy and were well tolerated. SS—OC/Brij and SS—OP/Brij LNPs were similar in efficacy and tolerability.

### Example 8: Characterization of LNPs comprising Myrj

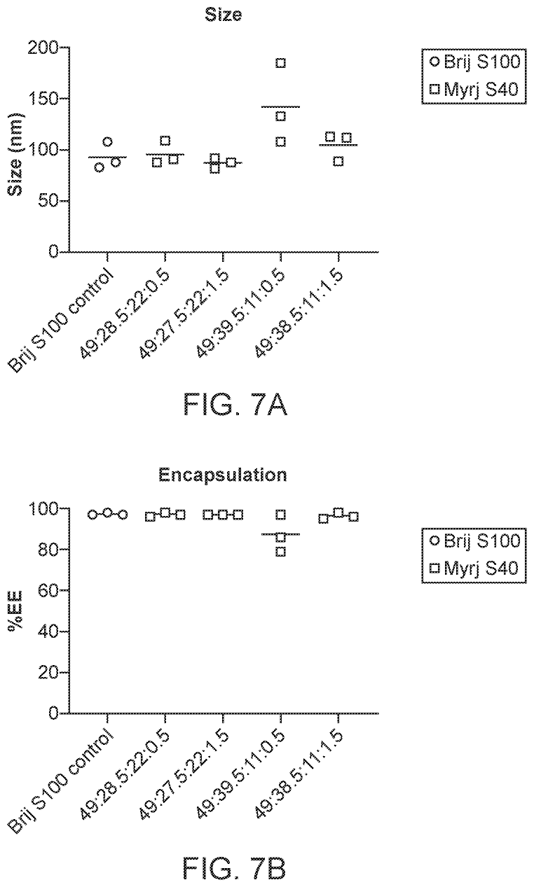

SVV-neg/OC:Cholesterol: DSPC:Myrj S40 (49:28.5:22:0.5 or 49:27.5:22:1.5 or 49:39.5:11:0.5 or 49:38.5:11:1.5 mol %) LNPs were prepared. A Brij S100 control was also included (49:28.5:22:0.5 mol % of OC:Chol:DSPC:Brij 5100). The N:P ratio was 14 noting 2 ionizable amines in SS—OC. Total lipid concentration was to 20 mM and the RNA acidifying buffer was 25 mM acetate pH 5. Formulations were mixed at a 3:1 aqueous:organic volume ratio at 12 mL/min without any heat during mixing. Formulations were dialyzed against 1X PBS pH 7.2 or 25 mM tris, 50 mM sucrose, 113 mM NaCl, pH 7.4 buffer for at least 18 hours. Formulations were concentrated using 100kD Amicon centrifugal filtration units. LNP sizes were measured by dynamic light scattering (FIG. 7A) and encapsulation efficiency was measured by RiboGreen (FIG. 7B). Each unique composition was formulated at least three times on separate days to ensure reproducibility.

The results showed that LNPs formulated using Myrj S40 as the PEG-lipid yielded similar size and encapsulation efficiency as compared to Brij S100 as the PEG-lipid, across the four molar compositions tested.

### Example 9: Formulation of Lipid Nanoparticles for Intravenous Delivery of CVA21-Encoding RNA

Recombinant RNA molecules comprising CVA21 genomes were formulated in lipid nanoparticles for delivery of the RNA in vivo.

Lipid nanoparticle production: Lipids (e.g., cationic lipid, PEG-lipid, helper lipid) used in the formulation of lipid nanoparticles are selected from the following:
D-Lin-MC3-DMA (MC3);
N-(2,3-dioleoyloxy)propyl)-N,N,N-trimethylammonium chloride (DOTAP)
COATSOME® SS-LC (former name: SS-18/4PE-13);
COATSOME® SS-EC (former name: SS-33/4PE-15);
COATSOME® SS—OC;
COATSOME® SS—OP;
Di((Z)-non-2-en-1-yl)₉-((4-dimethylamino)butanoyl)oxy)heptadecanedioate (L-319) cholesterol;
1,2-distearoyl-sn-glycero-3-phosphocholine (DSPC);
1,2-dilauroyl-sn-glycero-3-phosphoethanolamine (DLPE);
1,2-dioleoyl-sn-glycero-3-phosphocholine (DOPC);
1,2-dioleoyl-sn-glycero-3-phosphoethanolamine (DOPE);
1,2-distearoyl-sn-glycero-3-phosphoethanolamine-N-[amino(polyethyleneglycol)—5000] (DSPE-PEG5K);
1,2-dipalmitoyl-rac-glycerol methoxypolyethylene glycol-2000 (PEG2k-DPG);
1,2-distearoyl-rac-glycero-3-methylpolyoxyethylene-2000 (DSG-PEG2K);
1,2-dimyristoyl-rac-glycero-3-methylpolyoxyethylene-2000 (DMG-PEG2K) polyoxyethylene (100) stearyl ether (BRIJ S100; CAS number: 9005-00-9);
polyoxyethylene (20) stearyl ether (BRIJ S20; CAS number: 9005-00-9);
polyoxyethylene (20) oleyl ether (BRIJ 020; CAS number: 9004-98-2);
polyoxyethylene (20) cetyl ether (BRIJ C₂₀, CAS number: 9004-95-9);
Polyoxyethylene (40) stearate (MYRJ S40, CAS number: 9004-99-3).

Lipids were prepared in ethanol at various ratios. RNA lipid nanoparticles were then generated using microfluidic micromixture (Precision NanoSystems, Vancouver, BC) at a combined flow rate of 2 mL/min (0.5 mL/min for ethanol, lipid mix and 1.5 mL/min for aqueous buffer, RNA). The resulting particles were washed by tangential flow filtration with PBS containing Ca and Mg.

Analysis of physical characteristics of lipid nanoparticles: Physical characteristics of lipid nanoparticles were evaluated before and after tangential flow filtration. Particle size distribution and zeta potential measurements were determined by light scattering using a Malvern Nano-ZS Zetasizer (Malvern Instruments Ltd, Worcestershire, UK). Size measurements were performed in HBS at pH 7.4 and zeta potential measurements were performed in 0.01 M HBS at pH 7.4. Percentage of RNA entrapment was measured by Ribogreen assay. Lipid nanoparticles that showed greater than 80 percent RNA entrapment were tested in vivo.

### Example 10: In Vivo Studies of LNPs Comprising CVA21-RNA

The anti-tumor efficacy of Coxsackievirus A21 (CVA21)-RNA encapsulated in LNP was evaluated in vivo using a murine SK-MEL-28 melanoma model. For these experiments, the CVA21-RNA viral genomes were encapsulated in LNPs comprising a molar ratio of SS—OC:DSPC:Chol:BRIJ S100 of 49:22:28.5:0.5 mol %. In some embodiments, the size of LNPs was 94 nm; PDI was 0.19; and % EE was 91%.

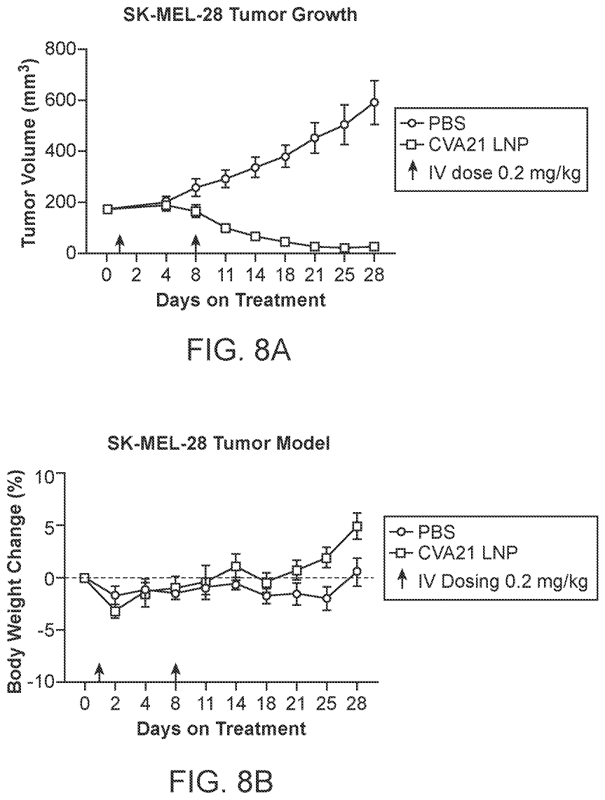

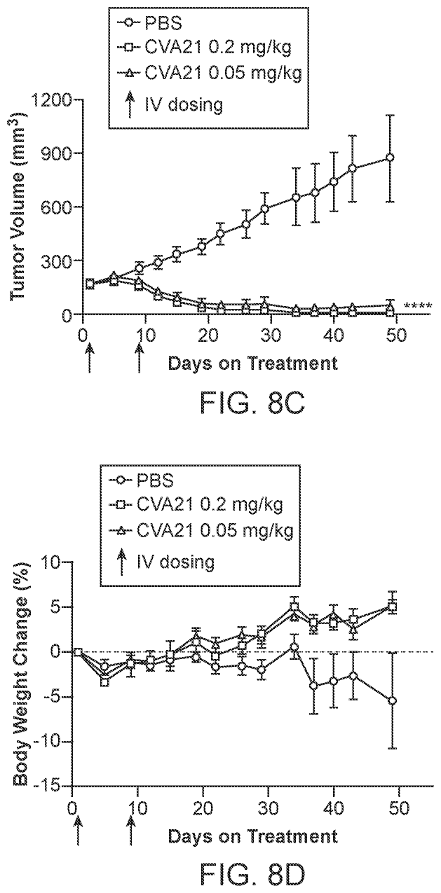

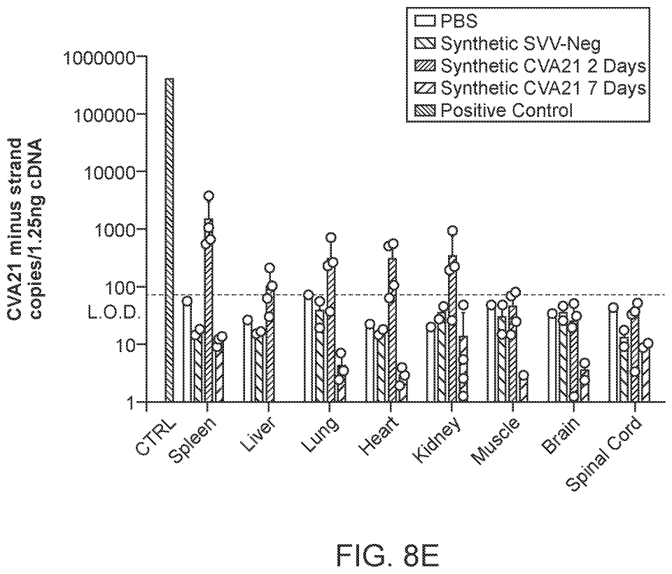

Briefly, athymic nude mice were subcutaneously implanted with SK-MEL-28 human melanoma tumor and treated on days 1 and 8 with IV administration of one of two doses of LNP comprising CVA21-RNA (0.2 mg/kg or 0.05 mg/kg). Tumor growth (FIG. 8A and FIG. 8C) and body weight changes (FIG. 8B and FIG. 8D) were monitored. PBS buffer was used as control.

Complete tumor regression at a dose level as low as 0.05 mg/kg was observed (FIG. 8B). Both doses were well tolerated, as indicated by stable body weight (FIG. 8B and FIG. 8D) and no adverse clinical signs. Low levels of CVA21 replication were detected by Rt-qPCR for CVA21 minus-strand and by plaque titer assay in spleen, liver, lung, heart, and kidney 2 days after injection. However, this was undetectable at 7 days (FIG. 8E) indicating that the mice had cleared the infection. The results showed that CVA21 encapsulated in LNPs comprising Brij S100 demonstrated high anti-tumor efficacy and was well tolerated.

### Example 11: Formulations of LNPs Comprising Different Ionizable Lipids

This example illustrates the encapsulation of non-replicating Seneca Valley virus (SVV) RNA (SVV-Neg) in LNP formulations. LNPs in this example comprise a lipid composition of ionizable lipid (CAT):DSPC:cholesterol:PEG2k-DMG at 50:7:40:3 mol %. The lipid mixture in ethanol was mixed with SVV-Neg in RNA acidifying buffer (50 mM citrate, pH 4) at a lipid-nitrogen-to-phosphate ratio (N:P) of 9 using a microfluidic device (Precision NanoSystems Inc.). Total lipid concentration was set to 20 mM.

LNPs were dialyzed against 50 mM phosphate, pH 6.0, for 12-16 h, and secondary dialysis was performed against 50 mM HEPES, 50 mM NaCl, 263 mM sucrose, pH 7.3, for 4-24 h at room temperature. Post-dialyzed LNPs were concentrated using 100 kDa AMICON® ULTRA CENTRIFUGAL filters (MilliporeSigma) and sterile filtered using 0.2 μm syringe filters. Samples were then characterized and diluted as needed. Upon dilution, a 5 w/v % glycerol spike was added if samples were then stored at −20° C.

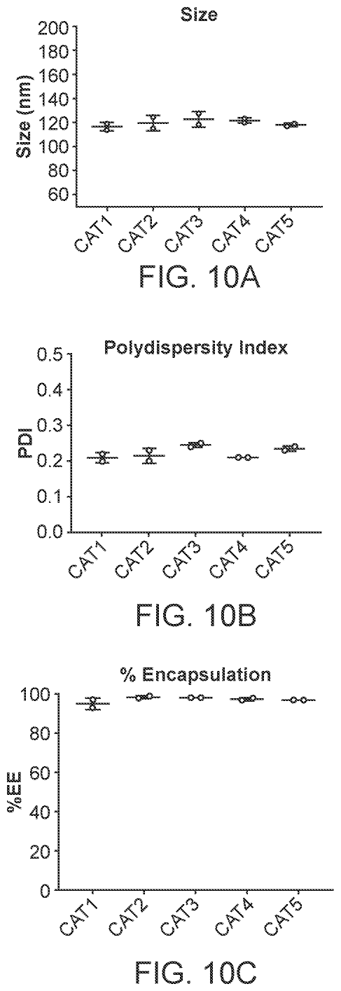

LNPs were characterized for particle size by dynamic light scattering (DLS) (FIG. 10A) and polydispersity index (PDI) (FIG. 10B). Encapsulation efficacy was measured using a fluorescence-based RiboGreen assay (FIG. 10C). Briefly, a standard curve was generated using the appropriate RNA; testing LNP samples were diluted 40X with TE buffer and evaluated to yield the amount of unencapsulated RNA (R_{f}) and diluted with Triton-X to generate the amount of total RNA (Rₜ). The difference between Rₜ and R_{f} over the total RNA (Rₜ) is the encapsulation efficiency (% EE):

% EE=(Rₜ−R_{f})/Rₜ×100%.

TABLE 7
LNP formulations and characterizations

| | Formulation Parameters | | | | | | | |
|---|---|---|---|---|---|---|---|---|
| | Ionizable | Lipid | Input RNA | | Characterization | | | |
| Ionizable | Lipid:Cholesterol:DSPC:PEG₂ₖ- | Conc. | Conc. | | Size | | [RNA] | |
| Lipid | DMG (mol %) | (mM) | (mg/mL) | N:P | (nm) | PDI | (μg/mL) | % EE |
| CAT1 | 50:40:7:3 | 20 | 0.119 | 9 | 114 | 0.20 | 274 | 97% |
| CAT2 | 50:40:7:3 | 20 | 0.119 | 9 | 115 | 0.20 | 265 | 99% |
| CAT3 | 50:40:7:3 | 20 | 0.119 | 9 | 118 | 0.24 | 259 | 98% |
| CAT4 | 50:40:7:3 | 20 | 0.119 | 9 | 123 | 0.21 | 261 | 98% |
| CAT5 | 50:40:7:3 | 20 | 0.119 | 9 | 119 | 0.24 | 254 | 97% |
| CAT1 | 50:40:7:3 | 20 | 0.119 | 9 | 119 | 0.22 | 233 | 93% |
| CAT2 | 50:40:7:3 | 20 | 0.119 | 9 | 124 | 0.23 | 284 | 98% |
| CAT3 | 50:40:7:3 | 20 | 0.119 | 9 | 127 | 0.25 | 236 | 98% |
| CAT4 | 50:40:7:3 | 20 | 0.119 | 9 | 120 | 0.21 | 220 | 97% |
| CAT5 | 50:40:7:3 | 20 | 0.119 | 9 | 117 | 0.23 | 238 | 97% |

### Example 12: Purified RNA Improves LNP Biophysical Properties

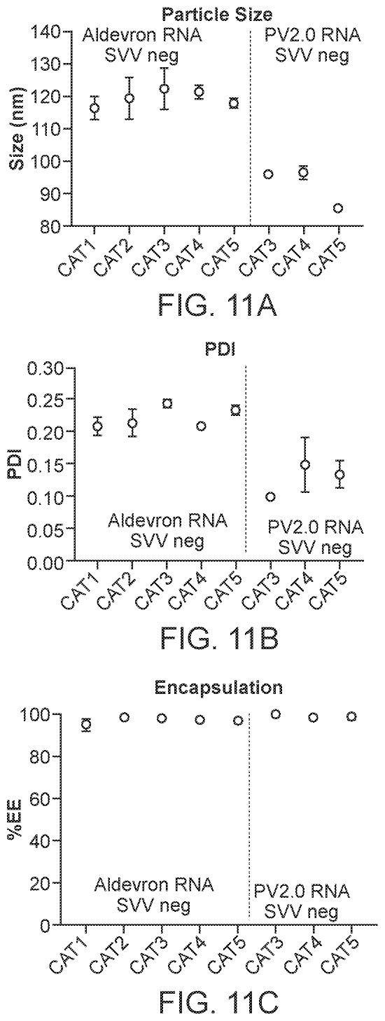

LNP formulations encapsulating SVV-Neg RNA were prepared and characterized as described in Example 11. The SVV-Neg RNA was purified using tangential flow filtration (TFF) or using oligo-dT chromatography and reverse phase chromatography. Tested LNP formulations encapsulating oligo-dT and reverse phase chromatography purified SVV-Neg RNA had reduced particle sizes and PDI (FIGS. 11A and 11B) with comparably high or further improved encapsulation efficiency (FIG. 11C).

### Example 13: Modification of RNA Acidifying Buffer Improves LNP Biophysical Properties

LNP formulations encapsulating SVV-Neg RNA were prepared and characterized as described in Example 11 but varying the RNA acidifying buffer to determine the effect changing the citrate concentration and pH would have on the LNP biophysical properties.

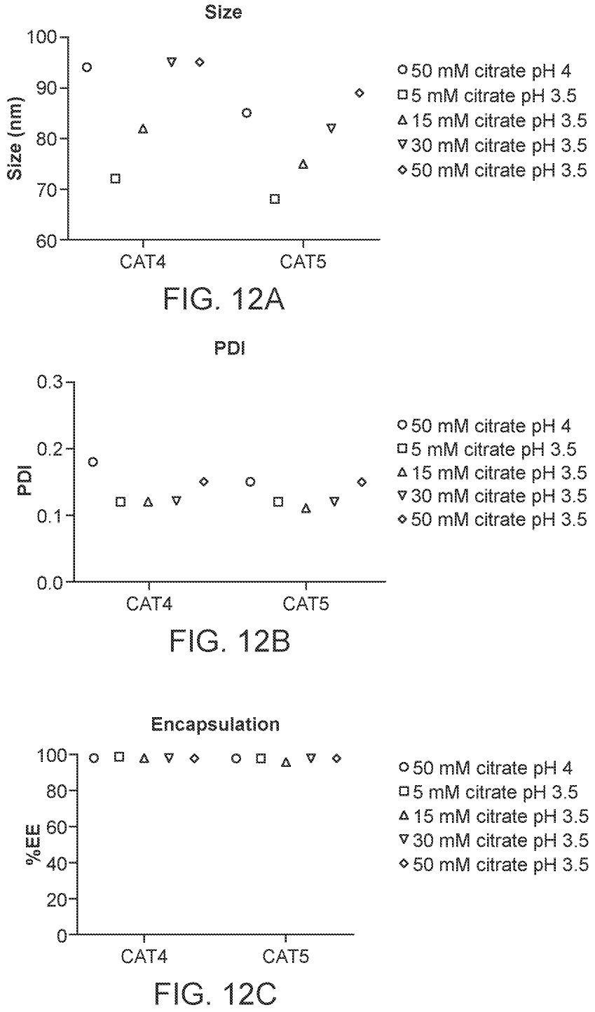

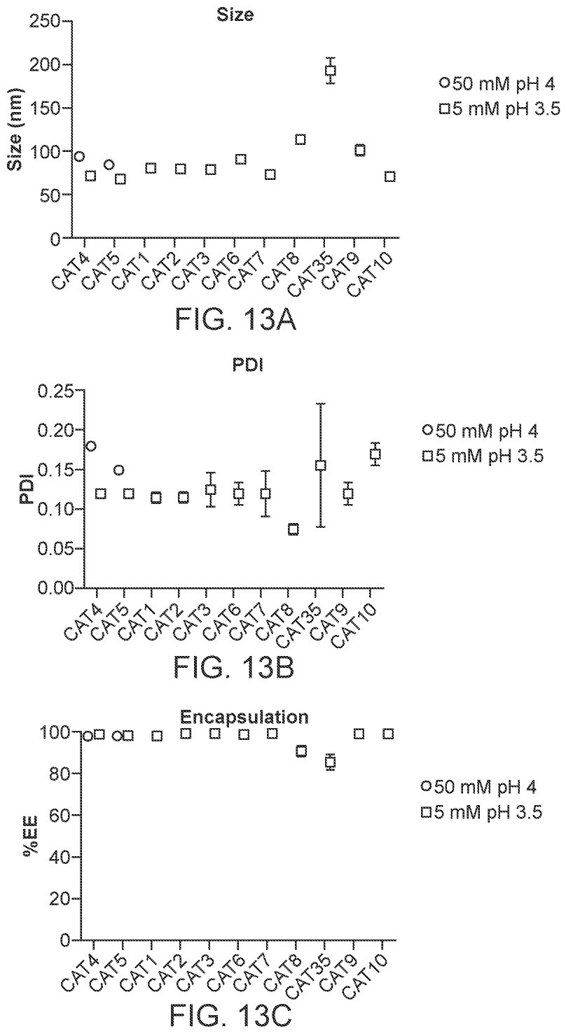

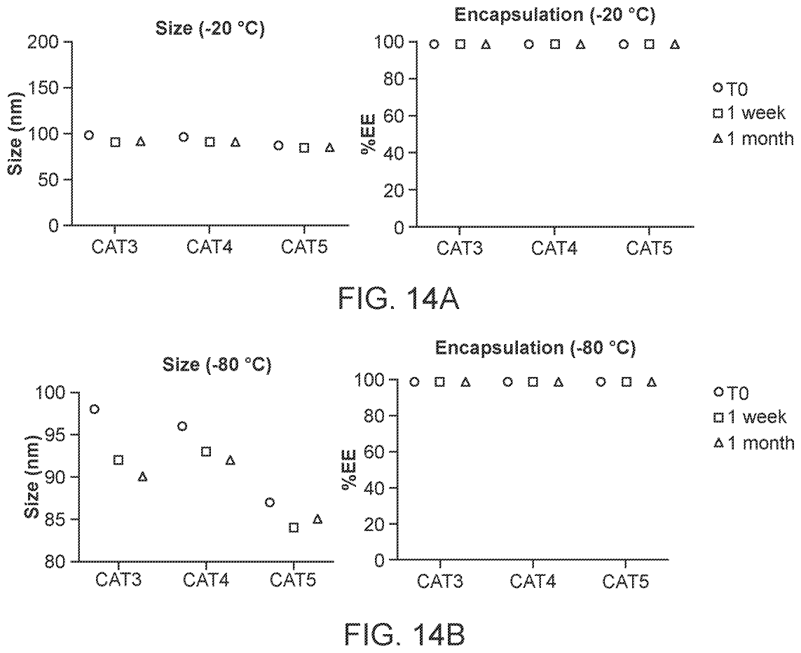

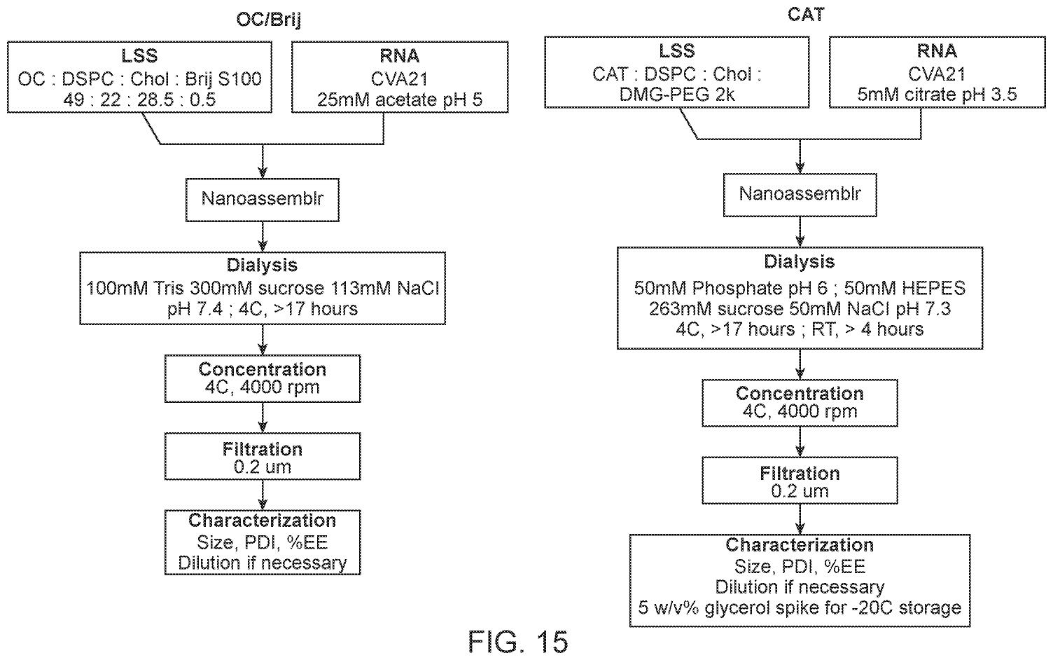

CAT4 and CAT5 formulations were tested with RNA acidifying buffer: (1) 50 nM citrate pH4; (2) 5 mM citrate pH 3.5; (3) 15 mM citrate pH 3.5; (4) 30 mM citrate pH 3.5; and (5) 50 mM citrate pH 3.5. FIGS. 12A, 12B, and 13C depict the particle size, PDI, and encapsulation efficiency of the LNPs. Further, CAT1 to CAT3, CAT6 to CAT10, and CAT35 LNP formulations were made with the 5 mM citrate pH 3.5 buffer (FIGS. 13A, 13B, and 13C).

The results suggested changing the RNA acidifying buffer (e.g., lowering salt concentration) resulted in smaller particle size and PDI.

### Example 14: LNP formulations Are Stable At Both −20° C. and −80° C.

CAT:DSPC:cholesterol:PEG2k-DMG (50:7:40:3 mol %) LNPs encapsulating SVV-neg RNA were prepared following similar procedures as in Example 11. The ionizable lipids tested were CAT3, CAT4, and CAT5. The RNA acidifying buffer used was 5 mM citrate, pH 3.5. Cryo-protectant (5 w/v % glycerol) was spiked into LNPs dilutions. The LNP formulations were then stored at −20° C. or −80° C. for one week or one month before the biophysical parameters were measured.

The results are shown in FIG. 14A (−20° C.) and FIG. 14B (−80° C.). Particle size and encapsulation efficiency remained the same for all formulations at −20° C. at the tested timepoints. Particle size decreased and encapsulation efficiency remained the same for all formulations at −80° C. at the tested timepoints.

### Example 15: In Vivo Studies of LNPs Comprising Different Ionizable Lipids

The in vivo pharmacodynamics and anti-tumor efficacy of Seneca Valley virus (SVV)—RNA encapsulated in LNP was evaluated in a mouse model for small cell lung cancer (SCLC).

In this example, the RNA molecules encoding SVV viral genomes and a NanoLuc luciferase (NLuc) were encapsulated in LNPs prepared according to Table 8 below. NLuc is a luciferase enzyme that produces luminescent signal when provided with the substrate furimazine. The LNPs were dialyzed overnight in 100 mM tris 300 mM sucrose 113 mM NaCl pH 7.4 at 5° C. Alternatively, the LNPs were dialyzed against 50 mM phosphate, pH 6.0, for 12-16 h and secondary dialysis was performed against 50 mM HEPES, 50 mM NaCl, 263 mM sucrose, pH 7.3, for 4-24 h at room temperature. Post-dialyzed LNP formulations were concentrated, filtered, characterized, and optionally diluted.

TABLE 8
LNP formulations for in vivo studies

| | | | Ionizable | | | | |
|---|---|---|---|---|---|---|---|
| | Ionizable | | lipid:Cholesterol:DSPC:PEG- | | Size | | |
| Formulation | Lipid | PEG-Lipid | lipid (mol %) | Acidifying Buffer | (nm) | PDI | % EE |
| CAT1/DMG | CAT1 | PEG₂ₖ-DMG | 50:40:7:3 | 5 mM citrate, | 75 | 0.13 | 97 |
| | | | | pH 3.5 | | | |
| CAT2/DMG | CAT2 | PEG₂ₖ-DMG | 50:40:7:3 | 5 mM citrate, | 73 | 0.18 | 98 |
| | | | | pH 3.5 | | | |
| CAT3/DMG | CAT3 | PEG₂ₖ-DMG | 50:40:7:3 | 5 mM citrate, | 74 | 0.17 | 98 |
| | | | | pH 3.5 | | | |
| CAT4/DMG | CAT4 | PEG₂ₖ-DMG | 50:40:7:3 | 5 mM citrate, | 80 | 0.16 | 97 |
| | | | | pH 3.5 | | | |
| CAT5/DMG | CAT5 | PEG₂ₖ-DMG | 50:40:7:3 | 5 mM citrate, | 73 | 0.18 | 96 |
| | | | | pH 3.5 | | | |
| CAT6/DMG | CAT6 | PEG₂ₖ-DMG | 50:40:7:3 | 5 mM citrate, | 86 | 0.2 | 98 |
| | | | | pH 3.5 | | | |
| CAT7/DMG | CAT7 | PEG₂ₖ-DMG | 50:40:7:3 | 5 mM citrate, | 72 | 0.14 | 97 |
| | | | | pH 3.5 | | | |
| CAT8/DMG | CAT8 | PEG₂ₖ-DMG | 50:40:7:3 | 5 mM citrate, | 120 | 0.07 | 90 |
| | | | | pH 3.5 | | | |
| CAT9/DMG | CAT9 | PEG₂ₖ-DMG | 50:40:7:3 | 5 mM citrate, | 87 | 0.19 | 98 |
| | | | | pH 3.5 | | | |
| CAT10/DMG | CAT10 | PEG₂ₖ-DMG | 50:40:7:3 | 5 mM citrate, | 71 | 0.21 | 97 |
| | | | | pH 3.5 | | | |
| CAT11/DMG | CAT11 | PEG₂ₖ-DMG | 50:40:7:3 | 5 mM citrate, | 81 | 0.17 | 96 |
| | | | | pH 3.5 | | | |
| CAT12/DMG | CAT12 | PEG₂ₖ-DMG | 50:40:7:3 | 5 mM citrate, | 86 | 0.33 | 89 |
| | | | | pH 3.5 | | | |
| CAT13/DMG | CAT13 | PEG₂ₖ-DMG | 50:40:7:3 | 5 mM citrate, | 79 | 0.12 | 97 |
| | | | | pH 3.5 | | | |
| CAT14/DMG | CAT14 | PEG₂ₖ-DMG | 50:40:7:3 | 5 mM citrate, | 81 | 0.32 | 91 |
| | | | | pH 3.5 | | | |
| CAT15/DMG | CAT15 | PEG₂ₖ-DMG | 50:40:7:3 | 5 mM citrate, | 110 | 0.24 | 98 |
| | | | | pH 3.5 | | | |
| CAT16/DMG | CAT16 | PEG₂ₖ-DMG | 50:40:7:3 | 5 mM citrate, | 75 | 0.2 | 95 |
| | | | | pH 3.5 | | | |
| CAT17/DMG | CAT17 | PEG₂ₖ-DMG | 50:40:7:3 | 5 mM citrate, | 73 | 0.52 | 97 |
| | | | | pH 3.5 | | | |
| CAT19/DMG | CAT19 | PEG₂ₖ-DMG | 50:40:7:3 | 5 mM citrate, | 67 | 0.21 | 98% |
| | | | | pH 3.5 | | | |
| CAT20/DMG | CAT20 | PEG₂ₖ-DMG | 50:40:7:3 | 5 mM citrate, | 66 | 0.21 | 98% |
| | | | | pH 3.5 | | | |
| CAT24/DMG | CAT24 | PEG₂ₖ-DMG | 50:40:7:3 | 5 mM citrate, | 64 | 0.37 | 97% |
| | | | | pH 3.5 | | | |
| CAT31/DMG | CAT31 | PEG₂ₖ-DMG | 50:40:7:3 | 5 mM citrate, | 67 | 0.17 | 98% |
| | | | | pH 3.5 | | | |
| CAT7/Brij | CAT7 | Brij S100 | 54.5:25:20:0.5 | 5 mM citrate, | 124 | 0.14 | 82% |
| | | | | pH 3.5 | | | |
| CAT18/DMG | CAT18 | PEG₂ₖ-DMG | 50:40:7:3 | 5 mM citrate, | 92 | 0.19 | 95 |
| | | | | pH 3.5 | | | |
| CAT21/DMG | CAT21 | PEG₂ₖ-DMG | 50:40:7:3 | 5 mM citrate, | 68 | 0.31 | 96 |
| | | | | pH 3.5 | | | |
| CAT22/DMG | CAT22 | PEG₂ₖ-DMG | 50:40:7:3 | 5 mM citrate, | 65 | 0.32 | 96 |
| | | | | pH 3.5 | | | |
| CAT23/DMG | CAT23 | PEG₂ₖ-DMG | 50:40:7:3 | 5 mM citrate, | 95 | 0.16 | 96 |
| | | | | pH 3.5 | | | |
| CAT25/DMG | CAT25 | PEG₂ₖ-DMG | 50:40:7:3 | 5 mM citrate, | 68 | 0.4 | 96 |
| | | | | pH 3.5 | | | |
| CAT26/DMG | CAT26 | PEG₂ₖ-DMG | 50:40:7:3 | 5 mM citrate, | 68 | 0.32 | 97 |
| | | | | pH 3.5 | | | |
| CAT27/DMG | CAT27 | PEG₂ₖ-DMG | 50:40:7:3 | 5 mM citrate, | 62 | 0.38 | 95 |
| | | | | pH 3.5 | | | |
| CAT28/DMG | CAT28 | PEG₂ₖ-DMG | 50:40:7:3 | 5 mM citrate, | 68 | 0.21 | 98 |
| | | | | pH 3.5 | | | |
| CAT29/DMG | CAT29 | PEG₂ₖ-DMG | 50:40:7:3 | 5 mM citrate, | 60 | 0.35 | 97 |
| | | | | pH 3.5 | | | |
| CAT30/DMG | CAT30 | PEG₂ₖ-DMG | 50:40:7:3 | 5 mM citrate, | 64 | 0.34 | 97 |
| | | | | pH 3.5 | | | |
| CAT32/DMG | CAT32 | PEG₂ₖ-DMG | 50:40:7:3 | 5 mM citrate, | 62 | 0.23 | 98 |
| | | | | pH 3.5 | | | |
| CAT34/DMG | CAT34 | PEG₂ₖ-DMG | 50:40:7:3 | 5 mM citrate, | 69 | 0.22 | 99 |
| | | | | pH 3.5 | | | |
| CAT7/DMG | CAT7 | PEG₂ₖ-DMG | 50:40:7:3 | 5 mM citrate, | 72 | 0.47 | 99 |
| | | | | pH 3.5 | | | |

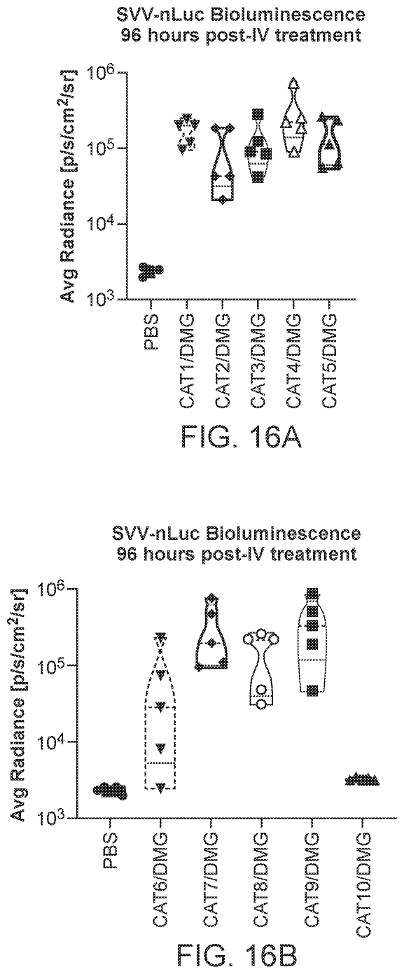

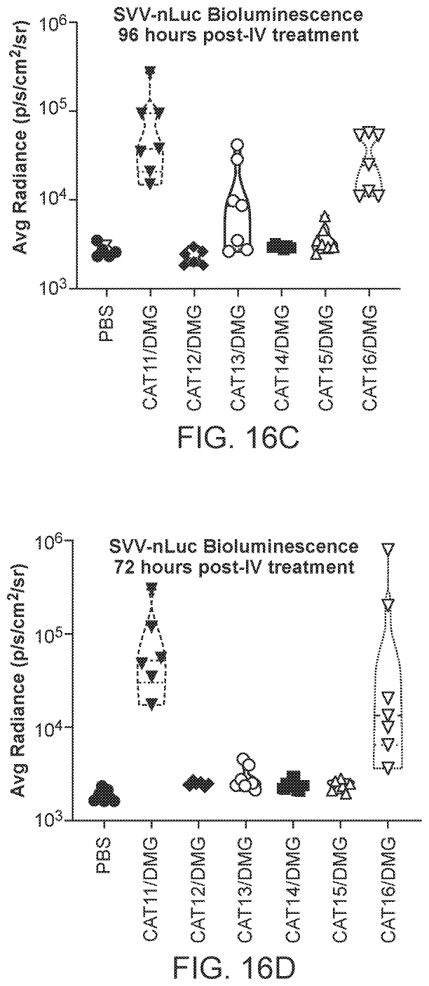

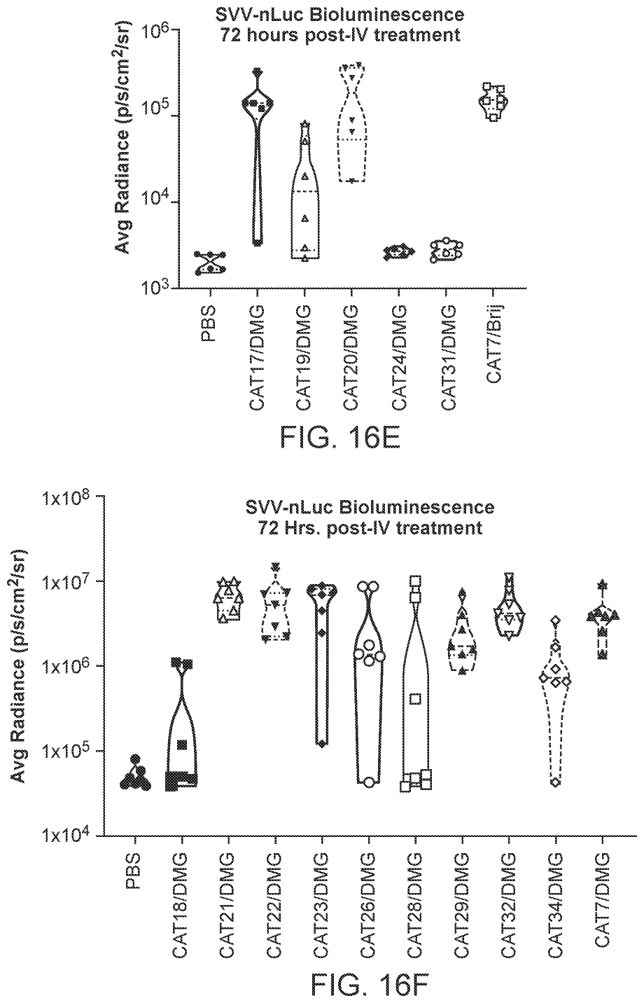

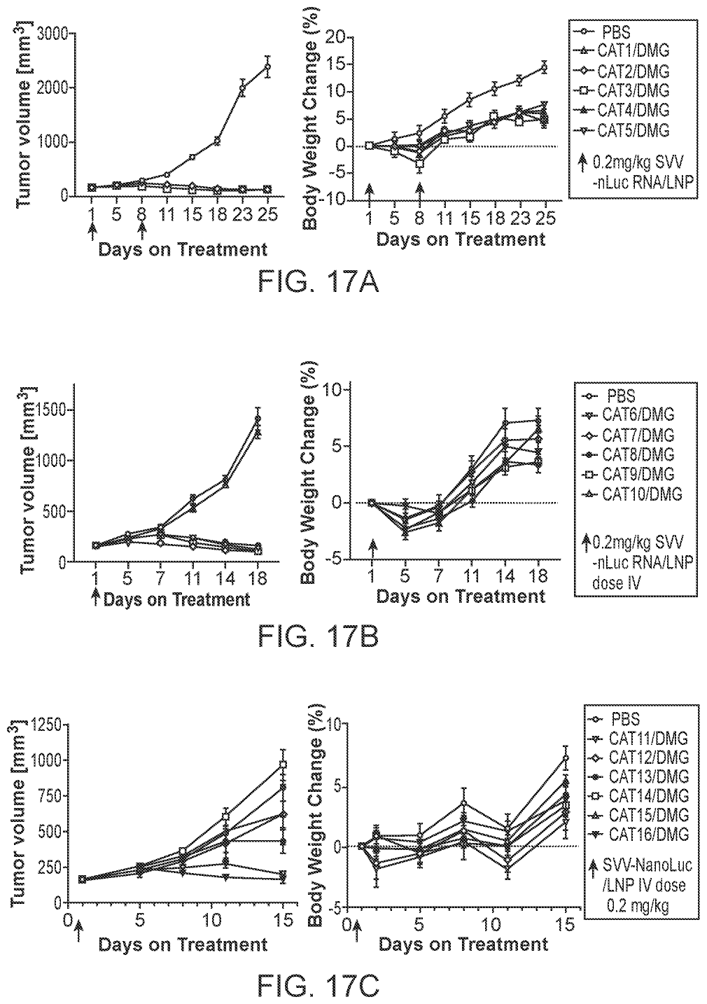

NCI-H446 human SCLC cells (5×10⁶ cells/0.I mL in a 1:1 mixture of serum-free PBS and Matrigel(R) were subcutaneously inoculated in the right flank of 8-week-old female athymic nude mice (Charles River Laboratories). When median tumor size reached approximately 150 mm³ (120-180 mm³ range), mice were intravenously administered 0.2 mg/kg of PBS or the LNPs comprising SVV-RNA on day 1 or on days 1 and 8. Bioluminescence (BLI) was assessed 96 h post-dose utilizing optical imagine IVIS Lumina (PerkinElmer), and the signal was quantified using Molecular Imaging software (FIGS. 16A-16F). Tumor volume and body weight were assessed 3 times per week (FIGS. 17A-17E).

Tumor regression after two 0.2 mg/kg doses was observed for the CAT1 to CAT5 formulations (FIG. 17A, left), and all formulations were well-tolerated (FIG. 17A, right). Tumor regression at a single 0.2 mg/kg dose was observed for the CAT6-CAT9, CAT11, CAT16-CAT17, CAT19-CAT24, CAT26, CAT29, CAT32, and CAT34 formulations (FIGS. 17B-17E, left), and all formulations were well-tolerated (FIGS. 17B-17E, right). Tumor growth inhibition was observed with CAT12-CAT13, CAT15, CAT18, and CAT28 formulations (FIGS. 17B-17E, left), and all formulations were well-tolerated (FIGS. 17B-17E, right).

### Example 16: In vivo Studies of LNPs Comprising CAT7 and Different PEG-Lipids

The in vivo pharmacodynamics and anti-tumor efficacy of SVV-RNA encapsulated in LNP with varying lipid compositions was evaluated in a mouse model for small cell lung cancer (SCLC). The RNA molecule encoding SVV viral genomes and NLuc were encapsulated in LNPs prepared according to Table 9 below, following a similar procedure described in Example 11. Total lipid concentration was set to 20 mM, and the lipid-nitrogen-to-phosphate ratio (N:P) was 9.

TABLE 9
LNP formulations for in vivo studies

| | | | Ionizable | | | | |
|---|---|---|---|---|---|---|---|
| | Ionizable | | lipid:Cholesterol:DSPC:PEG- | | Size | | |
| Formulation | Lipid | PEG-Lipid | lipid (mol %) | Acidifying Buffer | (nm) | PDI | % EE |
| CAT7/DMG_1 | CAT7 | PEG₂ₖ-DMG | 50:40:7:3 | 5 mM citrate, | 60.4 | 0.34 | 98 |
| | | | | pH 3.5 | | | |
| CAT7/CHM1_1 | CAT7 | CHM-001 | 54.6:25.1:20.1:0.25 | 5 mM citrate, | 123.3 | 0.09 | 97 |
| | | | | pH 3.5 | | | |
| CAT7/DMG_2 | CAT7 | PEG₂ₖ-DMG | 44.5:50:5:0.5 | 5 mM citrate, | 116.5 | 0.12 | 97 |
| | | | | pH 3.5 | | | |
| CAT7/DMG_3 | CAT7 | PEG₂ₖ-DMG | 40:50:8.75:1.25 | 5 mM citrate, | 66.3 | 0.14 | 98 |
| | | | | pH 3.5 | | | |
| CAT7/DMG_4 | CAT7 | PEG₂ₖ-DMG | 60:25:14.5:0.5 | 5 mM citrate, | 93.5 | 0.12 | 97 |
| | | | | pH 3.5 | | | |
| CAT7/DMG_5 | CAT7 | PEG₂ₖ-DMG | 60:34.3:5:0.7 | 5 mM citrate, | 117.9 | 0.16 | 97 |
| | | | | pH 3.5 | | | |
| CAT7/CHM6_1 | CAT7 | CHM-006 | 50:42.5:7:0.5 | 5 mM citrate, | 102.3 | 0.13 | 98 |
| | | | | pH 3.5 | | | |
| CAT7/CHM6_2 | CAT7 | CHM-006 | 58:33.5:7:1.5 | 5 mM citrate, | 103.3 | 0.2 | 96 |
| | | | | pH 3.5 | | | |
| CAT7/CHM6_3 | CAT7 | CHM-006 | 58:34.5:7:0.5 | 5 mM citrate, | 117.6 | 0.17 | 98 |
| | | | | pH 3.5 | | | |

The pharmacodynamics (assessed via a bioluminescence assay) and tumor growth inhibition ability of the SVV-NanoLuc-encapsulated LNPs was evaluated as described in Example 15.

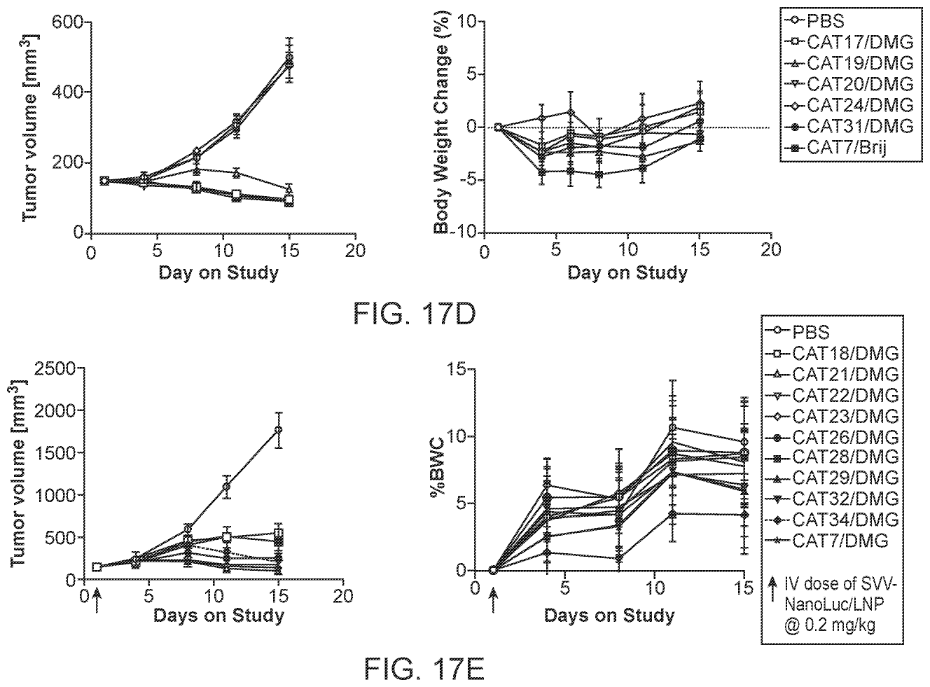

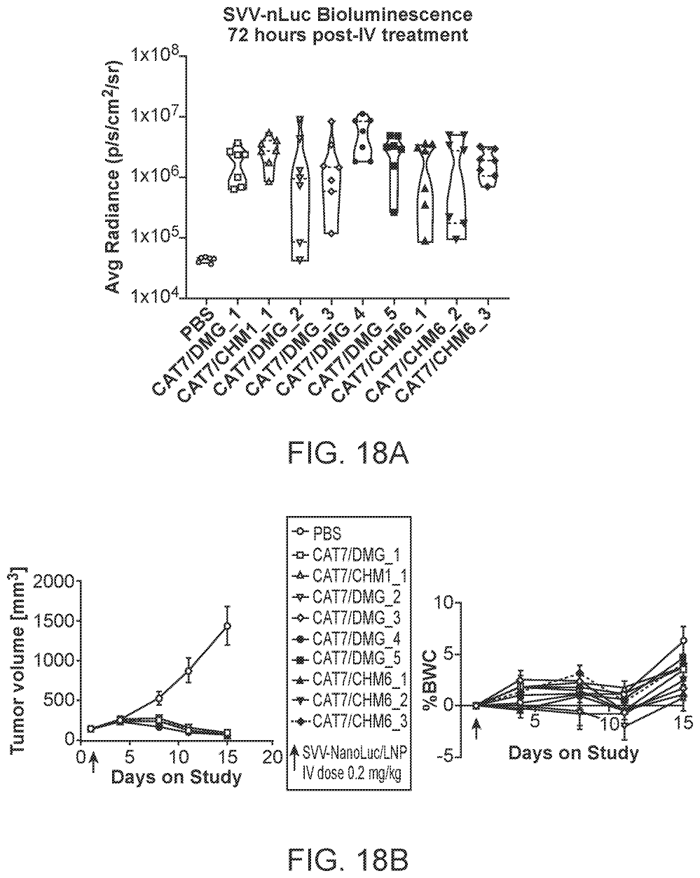

Nanoluciferase is detectable at 72 hours post-injection, indicative of continuous SVV (FIG. 18A). Complete tumor regression at a single 0.2 mg/kg dose was observed for all tested formulations, and all formulations were well-tolerated (FIG. 18B).

### Example 17: Pharmacokinetics Evaluation of LNP Formulations

The pharmacokinetics (PK) of Coxsackievirus A21 (CVA21)-RNA-encapsulating LNP formulations were evaluated in rats.

In this example, the RNA molecules encoding CVA-21 viral genomes were encapsulated in LNPs prepared according to Table 10 below, following the similar procedure as described in Example 11.

TABLE 10
LNP formulations for pharmacokinetics studies

| | | | Ionizable | | | | | | Dosing Schedule |
|---|---|---|---|---|---|---|---|---|---|
| | Ionizable | | lipid:DSPC:Cholesterol:PEG- | Acidifying | Size | | % | | (base on RNA |
| Formulation | Lipid | PEG-Lipid | lipid (mol %) | Buffer | (nm) | PDI | EE | Payload | conc.) |
| OC/CHM1 | Coatsome | CHM-001 | 49:22:28.5:0.5 | 25 mM | 83 | 0.25 | 97 | SVV-NEG- | 1 mg/kg, |
| | SS-OC | | | acetate, pH 5 | | | | RNA | Q2W2 |
| OC/CHM4 | Coatsome | CHM-004 | 49:22:28.5:0.5 | 25 mM | 77 | 0.18 | 98 | SVV-NEG- | 1 mg/kg, |
| | SS-OC | | | acetate, pH 5 | | | | RNA | Q2W2 |
| OC/CHM5 | Coatsome | CHM-005 | 49:22:28.5:0.5 | 25 mM | 74 | 0.2 | 99 | SVV-NEG- | 1 mg/kg, |
| | SS-OC | | | acetate, pH 5 | | | | RNA | Q2W2 |
| OC/CHM6 | Coatsome | CHM-006 | 49:22:28.5:0.5 | 25 mM | 84 | 0.2 | 98 | SVV-NEG- | 1 mg/kg, |
| | SS-OC | | | acetate, pH 5 | | | | RNA | Q2W2 |
| OC/CHM12 | Coatsome | CHM-012 | 49:22:28.5:0.5 | 25 mM | 73 | 0.15 | 99 | SVV-NEG- | 1 mg/kg, |
| | SS-OC | | | acetate, pH 5 | | | | RNA | Q2W2 |
| OC/Brij | Coatsome | Brij S100 | 49:22:28.5:0.5 | 25 mM | 69 | 0.18 | 98 | SVV-NEG- | 1 mg/kg, |
| | SS-OC | | | acetate, pH 5 | | | | RNA | Q1W2; |
| | | | | | | | | | 1 mg/kg, |
| | | | | | | | | | Q2W2 |
| OC/DMG | Coatsome | PEG₂ₖ- | 49:22:28.5:0.5 | 25 mM | 70 | 0.24 | 98 | SVV-NEG- | 1 mg/kg, |
| | SS-OC | DMG | | acetate, pH 5 | | | | RNA | Q2W2 |
| OC/DPG | Coatsome | PEG₂ₖ- | 49:22:28.5:0.5 | 25 mM | 69 | 0.21 | 98 | SVV-NEG- | 1 mg/kg, |
| | SS-OC | DMG | | acetate, pH 5 | | | | RNA | Q2W2 |
| OC/Brij | Coatsome | Brij S100 | 49:22:28.5:0.5 | 25 mM | 142 | 0.25 | 94 | CVA21- | 0.3 mg/kg, |
| | SS-OC | | | acetate, pH 5 | | | | RNA | Q2W2 |
| CAT7/DMG_6 | CAT7 | PEG₂ₖ- | 40:20:39.5:0.5 | 5 mM citrate, | 90.6 | 0.10 | 98 | CVA21- | 0.3 mg/kg, |
| | | DMG | | pH 3.5 | | | | RNA | Q2W2 |
| CAT7/DMG_3 | CAT7 | PEG₂ₖ- | 40:8.75:50:1.25 | 5 mM citrate, | 82.0 | 0.14 | 98 | CVA21- | 0.3 mg/kg, |
| | | DMG | | pH 3.5 | | | | RNA | Q2W2 |
| CAT7/DMG_5 | CAT7 | PEG₂ₖ- | 60:5:34.3:0.7 | 5 mM citrate, | 156.7 | 0.12 | 97 | CVA21- | 0.3 mg/kg, |
| | | DMG | | pH 3.5 | | | | RNA | Q2W2 |
| CAT7/CHM1_1 | CAT7 | CHM-001 | 54.6:20.1:25.1:0.25 | 5 mM citrate, | 106.9 | 0.11 | 97 | CVA21- | 0.3 mg/kg, |
| | | | | pH 3.5 | | | | RNA | Q2W2 |
| CAT7/CHM6_4 | CAT7 | CHM-006 | 50.1:7:42.6:0.25 | 5 mM citrate, | 131.1 | 0.10 | 98 | CVA21- | 0.3 mg/kg, |
| | | | | pH 3.5 | | | | RNA | Q2W2 |
| CAT7/Brij | CAT7 | Brij S100 | 54.5:20:25:0.5 | 5 mM citrate, | 116 | 0.2 | 88 | CVA21- | 0.3 mg/kg, |
| | | | | pH 3.5 | | | | RNA | Q2W2 |
| OC/DMG | Coatsome | PEG₂ₖ- | 49:22:28.5:0.5 | 25 mM | 110 | 0.28 | 94 | CVA21- | 0.3 mg/kg, |
| | SS-OC | DMG | | acetate, pH 5 | | | | RNA | Q2W2 |
| CAT7/CHM6_4 | CAT7 | CHM-006 | 50:7:40:3 | 5 mM citrate, | 102.6 | 0.37 | 98 | CVA21- | 0.3 mg/kg, |
| | | | | pH 3.5 | | | | RNA | Q2W2 |
| CAT11/DMG | CAT11 | PEG₂ₖ- | 50:7:40:3 | 5 mM citrate, | 79.8 | 0.31 | 98 | CVA21- | 0.3 mg/kg, |
| | | DMG | | pH 3.5 | | | | RNA | Q2W2 |
| CAT11/Brij | CAT11 | Brij S100 | 54.5:20:25:0.5 | 5 mM citrate, | 122.9 | 0.18 | 85 | CVA21- | 0.3 mg/kg, |
| | | | | pH 3.5 | | | | RNA | Q2W2 |

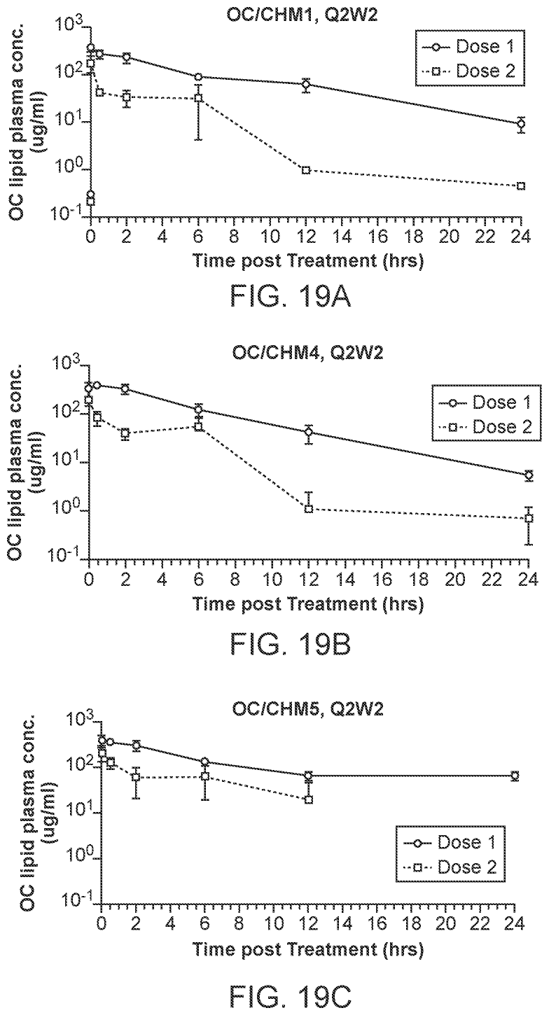

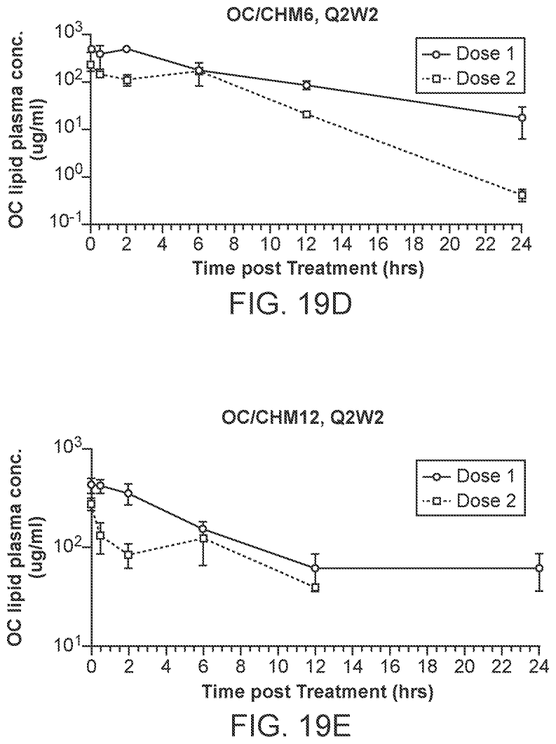

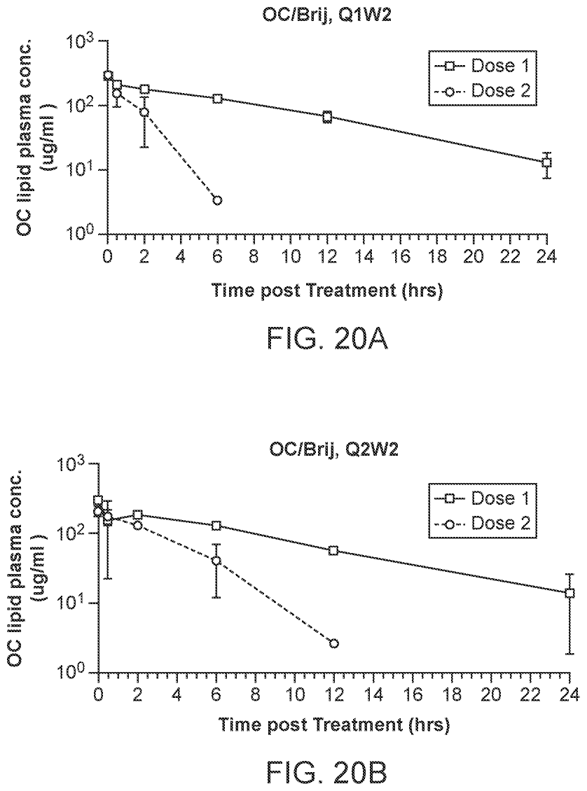

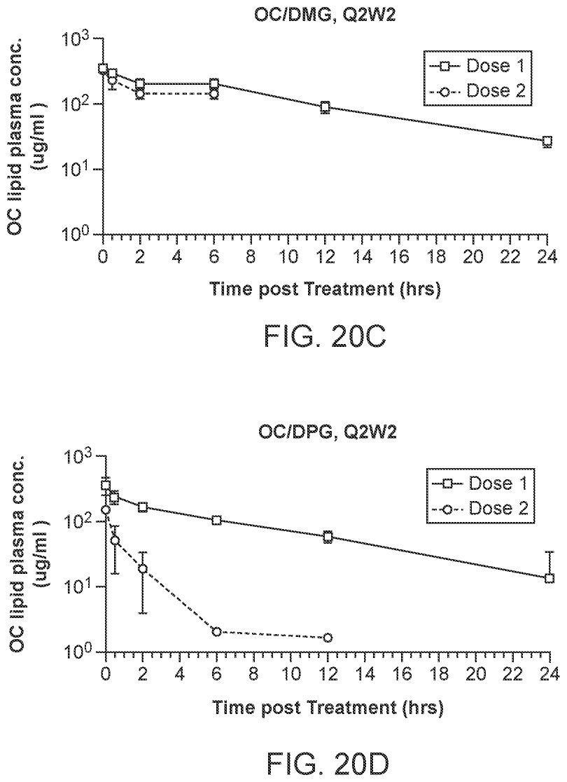

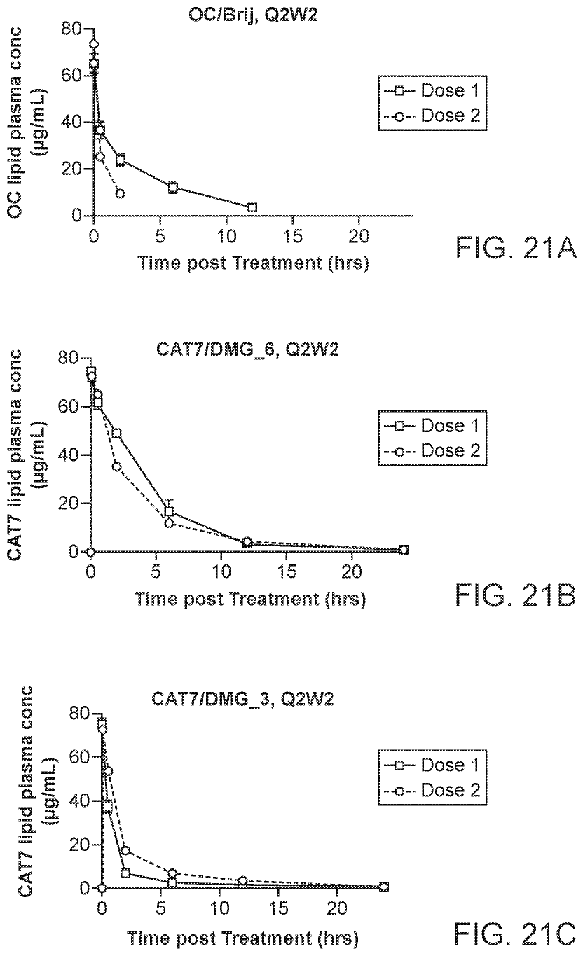

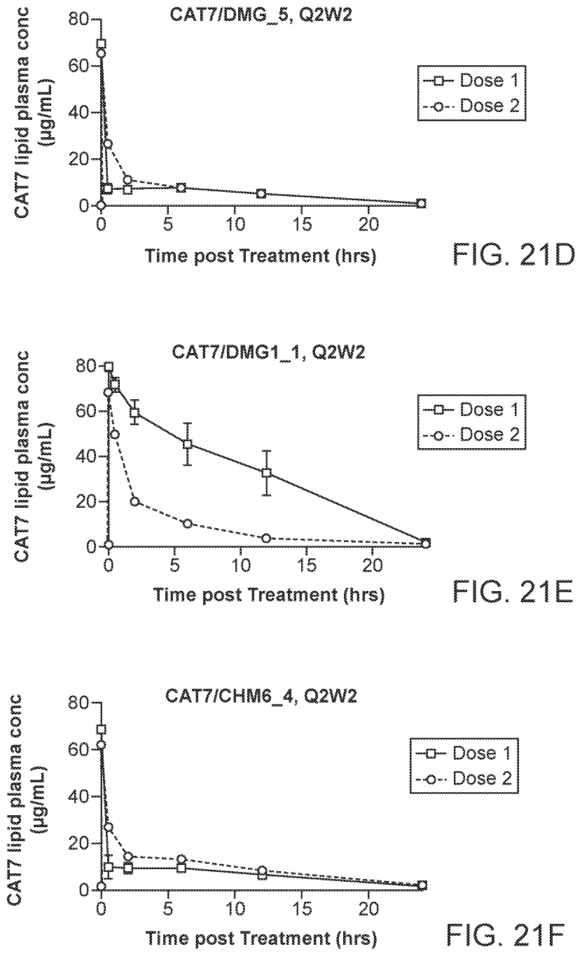

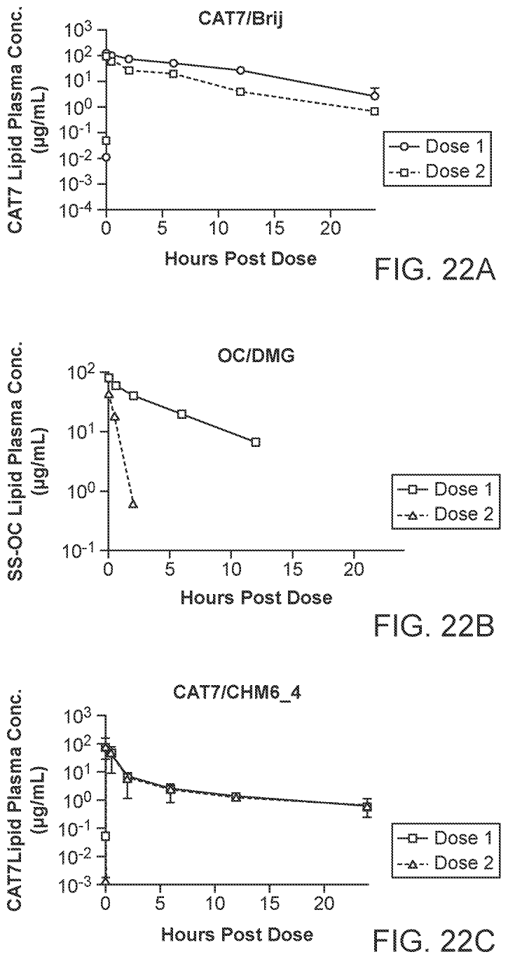

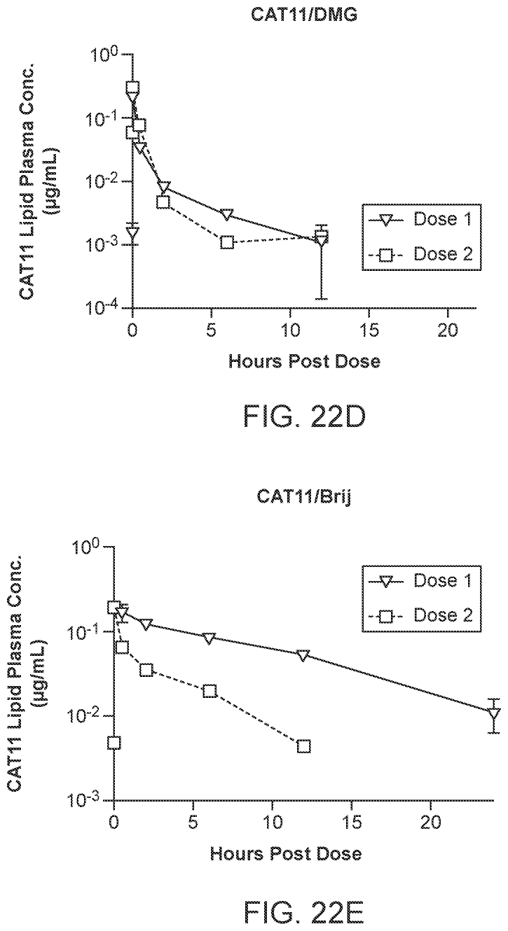

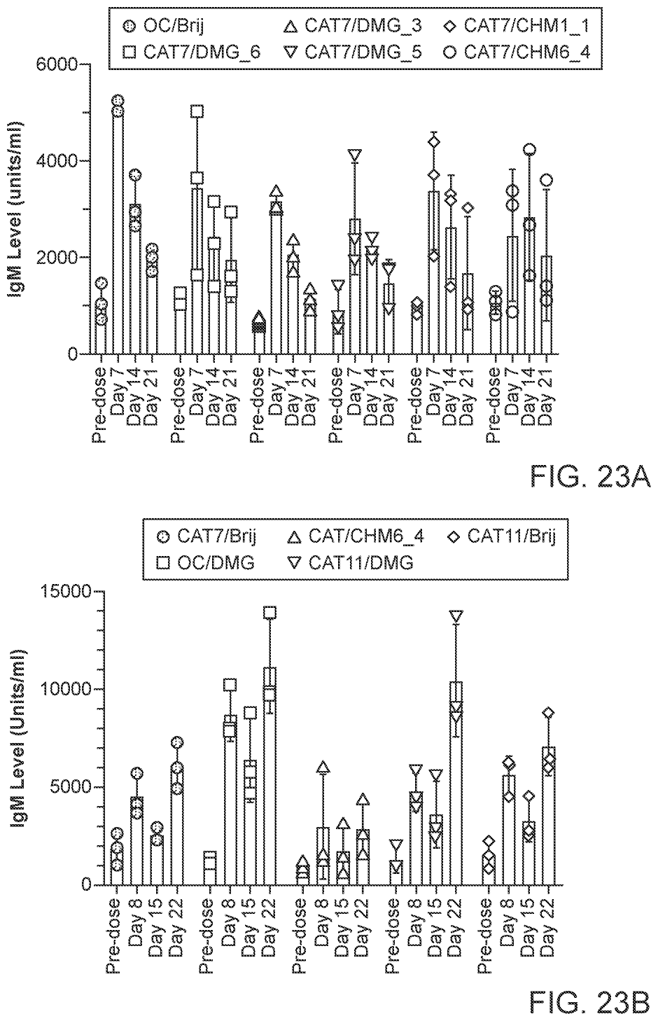

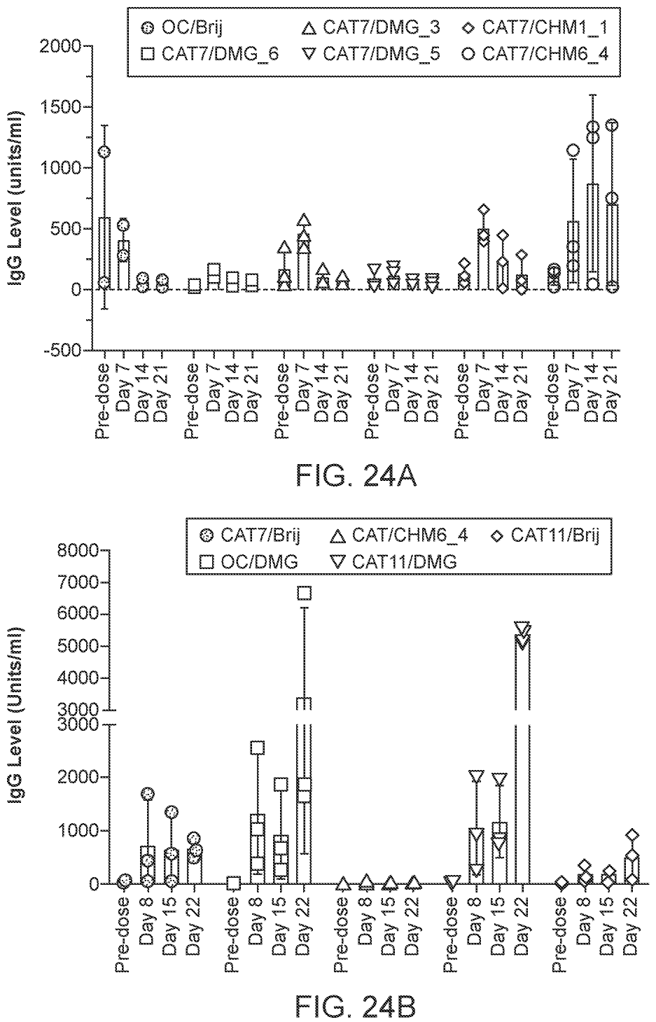

Naive female Sprague Dawley, JVC rats (age: 12 weeks) were intravenously administered 1 or 0.3 mg/kg of viral genomes comprised in the LNPs on days 1 and 15 (Q2W2) or on day 1 and day 8 (Q1W2). Plasma samples were collected at the predetermined times. The concentration of the ionizable lipid comprised in the LNPs (S-OC, CAT7, or CAT11) in plasma were measured by LC-MS (FIGS. 19A-19E, 20A-20D, 21A-21F, and 22A-22E) and the pharmacokinetics parameters were calculated and summarized in Table 11. IgM and IgG levels were analyzed by enzyme-linked immunoassay (ELISA) (FIGS. 23A-23B and FIGS. 24A-24B).

TABLE 11-1
Pharmacokinetics parameters

| | | | T_{1/2} | Tₘₐₓ | AUC_{INF} | AUC_{LAST} | C₀ | CL | Cₘₐₓ | Vₛₛ |
|---|---|---|---|---|---|---|---|---|---|---|
| Formulation | Dose # | Dose # | (h) | (h) | (h*μg/mL) | | (μg/mL) | (mL/h/kg) | (μg/mL) | (mL/kg) |
| OC/CHM1 | 1 | Day 1 | 4.92 | 0.02 | 1980.57 | 1915.72 | 368.81 | 6.06 | 364.94 | 43.17 |
| | 2 | Day 15 | 1.95 | 0.02 | 296.06 | 294.78 | 182.03 | 40.53 | 173.21 | 160.23 |
| OC/CHM4 | 1 | Day 1 | 3.51 | 0.5 | 2313.35 | 2284.79 | 344.1 | 5.19 | 401.04 | 27.54 |
| | 2 | Day 15 | 1.87 | 0.02 | 456.71 | 454.71 | 206.18 | 26.27 | 200.34 | 106.16 |
| OC/CHM5 | 1 | Day 1 | 4.54 | 0.02 | 2565.94 | 2499.28 | 404.68 | 4.68 | 403.35 | 30.67 |
| | 2 | Day 15 | 4.25 | 0.02 | 830.22 | 705.19 | 212.04 | 14.45 | 208.59 | 94.25 |
| OC/CHM6 | 1 | Day 1 | 5.04 | 0.02 | 3252.74 | 3130.05 | 477.19 | 3.69 | 472.62 | 26.03 |
| | 2 | Day 15 | 2.74 | 0.02 | 1240.69 | 1239.1 | 220.02 | 9.67 | 216.1 | 51.98 |
| OC/CHM12 | 1 | Day 1 | 4.3 | 0.02 | 2753.28 | 2692.79 | 431.4 | 4.36 | 431.02 | 26.56 |
| | 2 | Day 15 | 5.92 | 0.02 | 1459.81 | 1122.65 | 282.05 | 8.22 | 274.99 | 43.17 |
| OC/Brij | 1 | Day 1 | 6.25 | 0.02 | 2016.16 | 1890.9 | 306.42 | 5.95 | 299.62 | 51.87 |
| | 2 | Day 8 | 1.92 | 0.02 | 725.02 | 717.72 | 210.78 | 16.55 | 209.51 | 49.68 |
| OC/Brij | 1 | Day 1 | 5.89 | 0.02 | 2109.85 | 1998.58 | 278.63 | 5.69 | 276.04 | 48.38 |
| | 2 | Day 15 | 0.95 | 0.02 | 383.47 | 378.75 | 305.55 | 31.29 | 298.73 | 45.63 |
| OC/DMG | 1 | Day 1 | 7.1 | 0.02 | 2918.23 | 2641.81 | 346.95 | 4.11 | 345.08 | 42.14 |
| | 2 | Day 15 | 2.83 | 0.02 | 1020.25 | 783.99 | 321.7 | 11.76 | 318.13 | 47.55 |
| OC/DPG | 1 | Day 1 | 5.91 | 0.02 | 1929.25 | 1815.2 | 358.99 | 6.22 | 353.9 | 52.8 |
| | 2 | Day 15 | 1.96 | 0.02 | 140.25 | 135.55 | 155.02 | 85.56 | 149.29 | 202.16 |

| | Ionizable | | | | | | | |
|---|---|---|---|---|---|---|---|---|
| | Lipid Dose | | T_{1/2} | AUC₀₋₂ | AUC_{INF} | CL | Vₛₛ | Cₘₐₓ |
| Formulation | (mg/kg) | Dose # | (h) | h*μg/mL | | (mL/h/kg) | (mL/kg) | μg/mL |
| OC/Brij | 3.6 | Dose 1 | 2.8 | 69.01 | 183.79 | 19.59 | 81.85 | 65.06 |
| | 3.6 | Dose 2 | 0.6 | 42.84 | 48.22 | 74.65 | 65.31 | 73.5 |
| CAT7/DMG_6 | 6.5 | Dose 1 | 2.6 | 116.08 | 308.26 | 20.96 | 93.47 | 72.58 |
| | 6.5 | Dose 2 | 2.2 | 107.39 | 263.81 | 24.5 | 117.35 | 74.66 |
| CAT7/DMG_3 | 6.5 | Dose 1 | 0.6 | 54.21 | 95.67 | 67.55 | 275.46 | 75.79 |
| | 6.5 | Dose 2 | 1.8 | 79.42 | 181.21 | 35.66 | 185.34 | 73.04 |
| CAT7/DMG_5 | 6.5 | Dose 1 | 0.8 | 24.98 | 127.23 | 50.79 | 416.32 | 69.77 |
| | 6.5 | Dose 2 | 4.2 | 48.38 | 166.82 | 38.74 | 304.37 | 65.38 |
| CAT7/CHM1_1 | 6.5 | Dose 1 | 5 | 135.22 | 730.39 | 8.85 | 68.69 | 79.67 |
| | 6.5 | Dose 2 | 2.3 | 77.67 | 204.63 | 31.58 | 176.45 | 68.46 |
| CAT7/CHM6_4 | 6.5 | Dose 1 | 0.9 | 29.79 | 162.35 | 39.8 | 338.16 | 68.68 |
| | 6.5 | Dose 2 | 5.5 | 51.38 | 246.32 | 26.23 | 249.88 | 61.97 |
| CAT7/Brij | 6.5 | Dose 1 | 4.55 | 192.83 | 834.37 | 7.74 | 53.92 | 128.22 |
| | 6.5 | Dose 2 | 2.9 | 103.72 | 284.41 | 22.72 | 111.78 | 98.63 |
| OC/DMG | 3.6 | Dose 1 | 3.52 | 107.34 | 329.48 | 10.93 | 57.26 | 79.49 |
| | 3.6 | Dose 2 | 0.32 | 22.39 | 22.68 | 158.73 | 76.28 | 43.77 |
| CAT7/CHM6_4 | 6.5 | Dose 1 | 3.91 | 61.15 | 101.08 | 63.93 | 306.33 | 93.43 |
| | 6.5 | Dose 2 | 2.17 | 67.3 | 109.03 | 59.27 | 242.92 | 81.12 |
| CAT11/DMG | 6.6 | Dose 1 | 1.92 | 0.07 | 0.11 | 60435.32 | 138804.45 | 0.21 |
| | 6.6 | Dose 2 | 1.47 | 0.12 | 0.13 | 50064.5 | 47336.6 | 0.3 |
| CAT11/Brij | 6.6 | Dose 1 | 6.76 | 0.31 | 1.57 | 4203.47 | 38959.22 | 0.19 |
| | 6.6 | Dose 2 | 2.58 | 0.13 | 0.32 | 20537.25 | 82150.98 | 0.2 |

LNP formulations with different ratios and/or types of PEG-lipids display varying Ti/2, exposure, and clearance after multiple doses. These data indicate that the LNP compositions can be adapted to meet the need of various therapeutic payloads for long to short exposure.

Anti-PEG IgM level after dosing the LNP formulations was low and decreased from day 7 to 21 (FIG. 23A and FIG. 23B). Anti-PEG IgG was also low and did not significantly increase with multiple dose, indicating a low potential for immunogenicity (FIG. 24A and FIG. 24B). Among the tested formulations, LNPs comprising CAT7 as the ionizable lipid and CHM-006 as the PEG-lipid were observed with the lowest IgM and IgG levels.

### Example 18: Formulation of LNPs Encapsulating mRNA

SS—OC:Cholesterol:DSPC:PEG-lipid LNPs encapsulating mRNA at a N: P ratio of about 8:1 to 20:1 are prepared. The PEG-lipid is PEG2k-DPG, PEG2k-DMG or Brij S100. Total lipid concentration is about 10 to about 60 mM. Formulations are mixed and dialyzed, and concentrated. Size is measured by dynamic light scattering and encapsulation efficiency is measured by RiboGreen. The results show that Brij S100 could be used in replacement of PEG2k-DPG or PEG2k-DMG for mRNA LNP formulation.

mRNA LNP formulations in this Example are tested for pharmacokinetic characteristics upon repeat dosing via intravenous administration in mice. Copy number of RNA in serum post-dose is measured at predetermined time point. The results show that LNPs formulated using Brij 5100 exhibits a reduced clearance rate upon the second dose compared to LNPs formulated using PEG-2k DPG or PEG2k-DMG.

### Example 19: Formulated of LNPs Encapsulating mRNAs

This example illustrates the encapsulation of mRNAs in lipid nanoparticle (LNP) formulations. LNPs in this example comprise a lipid composition of CAT7: DSPC: cholesterol: CHM-006 at 54.5: 20: 25: 0.5 mol %. The lipid mixture in ethanol was mixed with human erythropoietin (hEPO) mRNAs or bi-specific T cell engager (BiTE)-encoding mRNAs in RNA acidifying buffer (5 mM citrate, pH 3.5). Total lipid concentration was set to 20 mM, and the lipid-nitrogen-to-phosphate ratio (N:P) was 9.

LNPs were dialyzed against 50 mM phosphate, pH 6.0, for 12-16 h and secondary dialysis was performed against 50 mM HEPES, 50 mM NaCl, 263 mM sucrose, pH 7.3, for 4-24 h at room temperature. Post-dialyzed LNPs were concentrated using 100 kDa AMICON® ULTRA CENTRIFUGAL filters (MilliporeSigma) and then sterile concentrated using 0.2 μm syringe filters. Samples were then characterized and diluted as needed. Upon dilution, a 5 w/v % glycerol spike was added if samples were stored at −20° C.

LNP sizes were measured by DLS, and the encapsulation efficacy was measured using a fluorescence-based RiboGreen assay (Table 12).

TABLE 12
LNP-formulated mRNAs

| | Ionizable Lipid | | CAT:DSPC:Cholesterol:PEG₂ₖ- | Size | | |
|---|---|---|---|---|---|---|
| mRNA | (CAT) | PEG-Lipid | DMG (mol %) | (nm) | PDI | % EE |
| hEPO | CAT7 | CHM-006 | 54.5:20:25:0.5 | 85 | 0.14 | 97 |
| BiTE | CAT7 | CHM-006 | 54.5:20:25:0.5 | 86 | 0.13 | 97 |
| hEPO | CAT7 | CHM-006 | 54.5:20:25:0.5 | 87 | 0.16 | 97 |
| BiTE | CAT7 | CHM-006 | 54.5:20:25:0.5 | 88.5 | 0.13 | 98 |

### Example 20: Pharmacokinetics of LNP-formulated mRNA

The PK of mRNA-encapsulating LNP formulations (Table 12) were evaluated in mice.

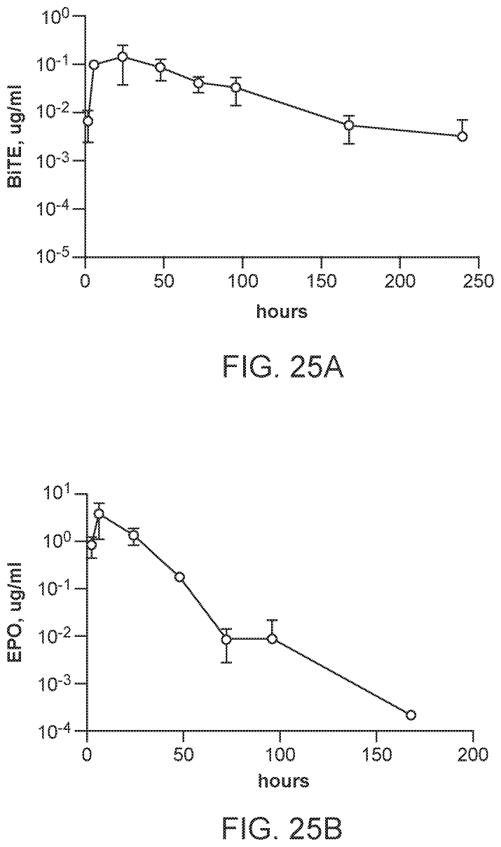

Naive female Balb/c mice were dosed with 1 mg/kg of the LNPs. 3 mice were bled at each predetermined timepoints and plasma was frozen at −80° C. for later analysis. Plasma levels of hEPO and BiTE were measured by Meso Scale Discovery (MSA) electrochemiluminescence (ECL) assays (FIG. 25A and FIG. 25B). High levels of protein expression and prolonged exposure were observed.

### Example 21: LNP-formulated RNAs with varying lengths

LNP formulations encapsulating RNA with various lengths were prepared according to Table 13 below, following a similar procedure as described in Example 11.

TABLE 13
LNP formulations

| RNA | | | | Ionizable | | | | |
|---|---|---|---|---|---|---|---|---|
| length | | Ionizable | | lipid:DSPC:Cholesterol:PEG- | Acidifying | Size | | |
| (kb) | Formulation | Lipid | PEG-Lipid | lipid (mol %) | Buffer | (nm) | PDI | % EE |
| 5.9 | OC/Brij | Coatsome | Brij S100 | 49:22:28.5:0.5 | 25 mM acetate, | 77.9 | 0.26 | 98% |
| | | SS-OC | | | pH 5 | | | |
| 14.2 | OC/Brij | Coatsome | Brij S100 | 49:22:28.5:0.5 | 25 mM acetate, | 93.5 | 0.23 | 95% |
| | | SS-OC | | | pH 5 | | | |
| 12.6 | OC/Brij | Coatsome | Brij S100 | 49:22:28.5:0.5 | 25 mM acetate, | 131.9 | 0.32 | 92% |
| | | SS-OC | | | pH 5 | | | |
| 5.9 | CAT7/DMG | CAT7 | PEG₂ₖ-DMG | 54.5:20:25:0.5 | 5 mM citrate, | 80.4 | 0.05 | 99% |
| | | | | | pH 3.5 | | | |
| 14.2 | CAT7/DMG | CAT7 | PEG₂ₖ-DMG | 54.5:20:25:0.5 | 5 mM citrate, | 99.8 | 0.22 | 93% |
| | | | | | pH 3.5 | | | |
| 12.6 | CAT7/DMG | CAT7 | PEG₂ₖ-DMG | 54.5:20:25:0.5 | 5 mM citrate, | 99.3 | 0.21 | 93% |
| | | | | | pH 3.5 | | | |

The data show that LNPs maintained good biophysical properties (e.g., small size and PDI, high % EE) despite the variable length of the encapsulated RNA.

### Example 22: Formulation Studies and Modeling of LNPs Comprising CAT7

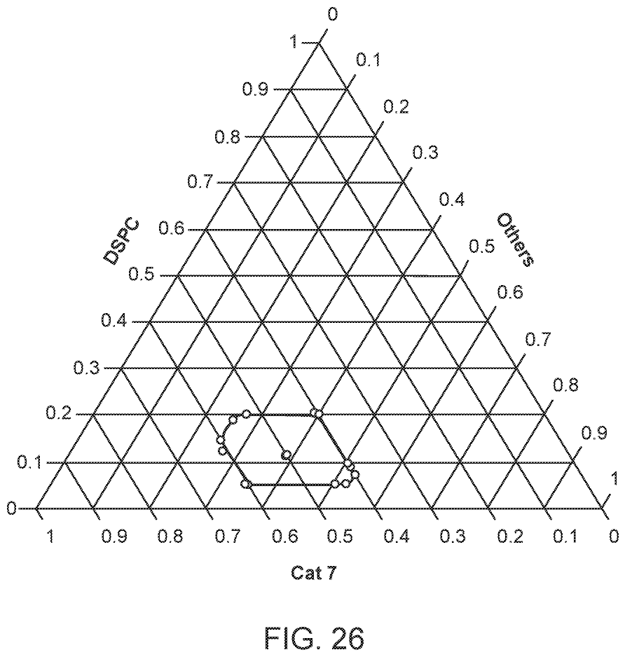

A-optimal criterion (Jones et al. 2021) was used to design formulation studies of LNPs comprising CAT7 (FIG. 26) and yielded 20 design of experiment (DOE) runs (Table 14). The total lipid concentration was set to 20 mM and the N:P ratio to 9. The design space tested LNPs comprising 40-60 mol % ionizable lipid of CAT7, 5-20 mol % helper lipid of DSPC, 25-50 mol % structural lipid of cholesterol, and 0.25-3% PEG-lipid of DMG-PEG2000 or CHM-001.

TABLE 14
Design of Experiment for CAT7 LNPs

| DOE Run | Composition | Mol % |
|---|---|---|
| 1 | ONC-SM-004:DSPC:Cholesterol:PEG_{2K}-DMG | 50:11.25:36.75:2 |
| 2 | ONC-SM-004:DSPC:Cholesterol:PEG₂ₖ-DMG | 40:20:39.5:0.5 |
| 3 | ONC-SM-004:DSPC:Cholesterol:PEG_{2K}-DMG | 60:5:32:3 |
| 4 | ONC-SM-004:DSPC:Cholesterol:CHM-001 | 60.9:12.2:25.4:1.5 |
| 5 | ONC-SM-004:DSPC:Cholesterol:CHM-001 | 40.1:9.5:50.1:0.25 |
| 6 | ONC-SM-004:DSPC:Cholesterol:PEG_{2K}-DMG | 44.5:5:50:0.5 |
| 7 | ONC-SM-004:DSPC:Cholesterol:PEG_{2K}-DMG | 40:8.75:50:1.25 |
| 8 | ONC-SM-004:DSPC:Cholesterol:CHM-001 | 60.5:5.0:33.5:0.88 |
| 9 | ONC-SM-004:DSPC:Cholesterol:PEG_{2K}-DMG | 60:14.5:25:0.5 |
| 10 | ONC-SM-004:DSPC:Cholesterol:CHM-001 | 40.4:20.2:38.6:0.83 |
| 11 | ONC-SM-004:DSPC:Cholesterol:PEG₂ₖ-DMG | 52.75:20:25:2.25 |
| 12 | ONC-SM-004:DSPC:Cholesterol:CHM-001 | 42.6:5.1:50.8:1.52 |
| 13 | ONC-SM-004:DSPC:Cholesterol:CHM-001 | 55.7:18.9:25.1:0.25 |
| 14 | ONC-SM-004:DSPC:Cholesterol:CHM-001 | 50.4:11.1:37.2:1.27 |
| 15 | ONC-SM-004:DSPC:Cholesterol:PEG_{2K}-DMG | 40:7:50:3 |
| 16 | ONC-SM-004:DSPC:Cholesterol:CHM-001 | 60.2:5:34.6:0.25 |
| 17 | ONC-SM-004:DSPC:Cholesterol:PEG_{2K}-DMG | 40:20:37:3 |
| 18 | ONC-SM-004:DSPC:Cholesterol:CHM-001 | 40.1:9.5:50.1:0.25 |
| 19 | ONC-SM-004:DSPC:Cholesterol:PEG_{2K}-DMG | 60:5:34.3:0.7 |
| 20 | ONC-SM-004:DSPC:Cholesterol:PEG_{2K}-DMG | 40.6:20.3:37.6:1.52 |

Within the parameters of the reliable design space, the DOE optimal composition was determined to be CAT7: DSPC: Cholesterol: PEG-lipid with the mol % ratio of 54.5: 20 25: 0.5.

A Self-Validated Ensemble Modeling (SVEM) method (Lemkus et al. 2021) was used to formulate a model for predicting biophysical characteristics of LNPs with varying compositions and identifying and fine-tuning LNP systems for different desired outcomes. In developing the model, the aim was to minimize PDI (weighted as 1) and size (weighted as 0.1).

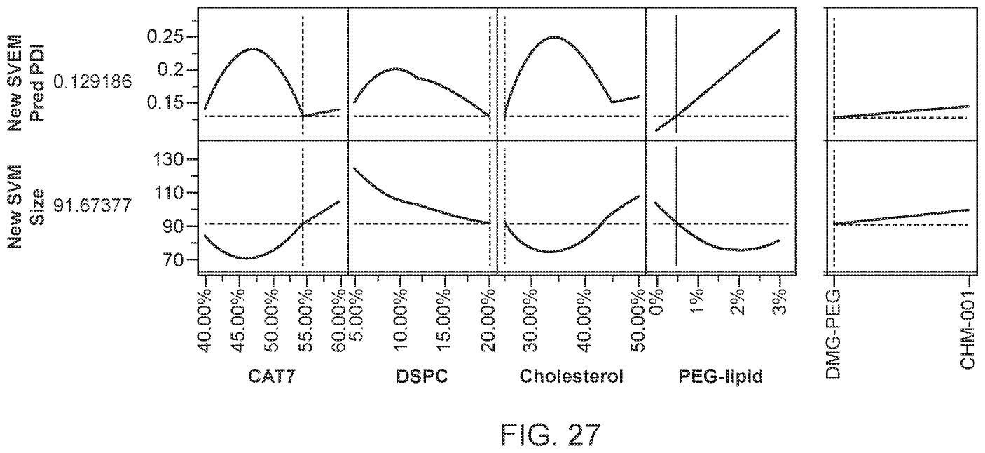

The resulting prediction profilers are shown in FIG. 27. Quadratic (curvature or non-linear) relationships are seen for CAT7, DSPC, and Cholesterol. CAT7 composition seems to significantly impact the PDI, with an increasing trend initially starting from 40 mol %, followed by a downward trend which stabilized at −55 mol %. Higher DSPC seems to favor a drop in both PDI and the size. Cholesterol follows a pattern very similar to CAT7 for both PDI and the size, but the model picks a lower molar composition. Increasing PEG-lipid composition is associated with a steep increase in observed PDI.

### Equivalents and Scope

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The present disclosure includes embodiments in which exactly one member of the group is present in, employed in, or otherwise relevant to a given product or process. The present disclosure includes embodiments in which more than one, or all of the group members are present in, employed in, or otherwise relevant to a given product or process.

Furthermore, the present disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the present disclosure, or aspects of the present disclosure, is/are referred to as comprising particular elements and/or features, certain embodiments of the present disclosure or aspects of the present disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the present disclosure, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the present disclosure can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present disclosure, as defined in the following claims.

1. A compound of Formula (I): 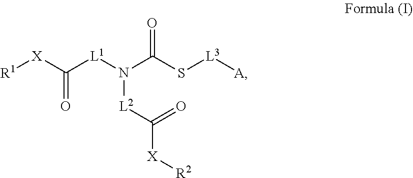
or a pharmaceutically acceptable salt or solvate thereof, wherein:
A is —N(CH₂R^{N1})(CH₂R^{N2}) or a 4-7-membered heterocyclyl ring containing at least one N, wherein the 4-7-membered heterocyclyl ring is optionally substituted with 0-6 R³;
each X is independently —O—, —N(R′)—, or —N(R²)—
R¹ is selected from the group consisting of optionally substituted C₁-C₃₁ aliphatic and steroidyl;
R² is selected from the group consisting of optionally substituted C₁-C₃₁ aliphatic and steroidyl;
R³ is optionally substituted C₁-C₆ aliphatic;
R^{N1} and R^{N2} are each independently hydrogen, hydroxy-C₁-C₆ alkyl, C₂-C₆ alkenyl, or a C₃-C₇ cycloalkyl;
L¹ is selected from the group consisting of an optionally substituted C₁-C₂₀ alkylene chain and a bivalent optionally substituted C₂-C₂₀ alkenylene chain;
L² is selected from the group consisting of an optionally substituted C₁-C₂₀ alkylene chain and a bivalent optionally substituted C₂-C₂₀ alkenylene chain; and
L³ is a bond, an optionally substituted C₁-C₆ alkylene chain, or a bivalent optionally substituted C₃-C₇ cycloalkylene; and
with the proviso that when A is —N(CH₃)(CH₃) and X is 0, L³ is not an C₁-C₆ alkylene chain.

2. The compound of claim 1, wherein R¹ and R² are each independently optionally substituted C₁-C₃₁ alkyl or optionally substituted C₂-C₃₁ alkenyl.

3. The compound of claim 1 or 2, wherein R¹ and R² are the same.

4. The compound of any of claims 1-3, wherein R¹ and R² are each independently optionally substituted C₁₀-C₂₀ alkyl.

5. The compound of any of claims 1-4, wherein R¹ and R² are each independently branched C₁₀-C₂₀ alkyl.

6. The compound of claim 1 or 2, wherein R¹ and R² are the different.

7. The compound of any of claims 1, 2, and 6, wherein R¹ is optionally substituted C₆-C₂₀ alkenyl and R² is optionally substituted C₁₀-C₂₀ alkyl.

8. The compound of any of claims 1, 2, 6, and 7, wherein R¹ is C₆-C₂₀ alkenyl and R² is branched C₁₀-C₂₀ alkyl.

9. The compound of any of claims 1-8, wherein L¹ is an optionally substituted C₁-C₁₀ alkylene chain and L² is an optionally substituted C₁-C₁₀ alkylene chain.

10. The compound of any of claims 1-9, wherein L¹ is an optionally substituted C₁-C₅ alkylene chain and L² is an optionally substituted C₁-C₅ alkylene chain.

11. The compound of any of claims 1-10, wherein L¹ is an optionally substituted C₁-C₃ alkylene chain and L² is an optionally substituted C₁-C₃ alkylene chain.

12. The compound of any of claims 1-11, wherein L¹ and L² are each —CH₂CH₂CH₂—.

13. The compound of any of claims 1-12, wherein L³ is a C₁-C₃ alkylene chain.

14. The compound of any of claims 1-12, wherein L³ is a bond.

15. The compound of any of claims 1-12, wherein L³ is a bivalent C₃-C₇ cycloalkylene.

16. The compound of any of claims 1-15, wherein the number of carbon atoms between the S of the thiolate and the closest N comprised in A is 2-10.

17. The compound of any of claims 1-16, wherein the number of carbon atoms between the S of the thiolate and the closest N comprised in A is 2-8.

18. The compound of any of claims 1-17, wherein the number of carbon atoms between the S of the thiolate and the closest N comprised in A is 2-5.

19. The compound of any of claims 1-18, wherein the number of carbon atoms between the S of the thiolate and the closest N comprised in A is 2-4.

20. The compound of any of claims 1-19, wherein the number of carbon atoms between the S of the thiolate and the closest N comprised in A is 3.

21. The compound of any one of claims 1-20, wherein the compound is a compound of Formula (I-a): 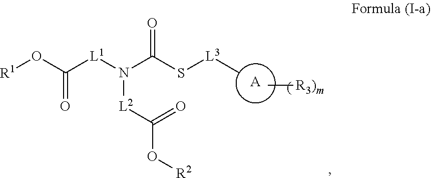
or a pharmaceutically acceptable salt or solvate thereof, wherein:
m is 0, 1, 2, 3, 4, 5, or 6.

22. The compound of claim 21, wherein A contains one or more S.

23. The compound of claim 21 or 22, wherein A is an optionally substituted 4-7-membered heterocyclyl ring containing exactly one N.

24. The compound of any one of claims 21-23, wherein A is an optionally substituted 5-6-membered heterocyclyl ring.

25. The compound of any one of claims 21-24, wherein A is an optionally substituted 6 membered heterocyclyl ring containing exactly one N.

26. The compound of any of claims 21-25, wherein the compound is a compound of Formula (I-b): 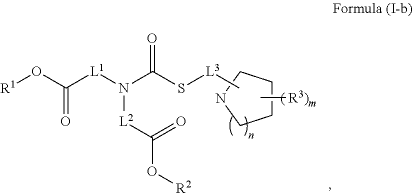
or a pharmaceutically acceptable salt or solvate thereof, wherein:
n is 0, 1, 2, or 3; and
m is 0, 1, 2, 3, 4, 5, or 6.

27. The compound of any of claims 21-26, wherein A is a tertiary amine.

28. The compound of any of claims 21-27, wherein the compound is a compound of Formula (I-bii): 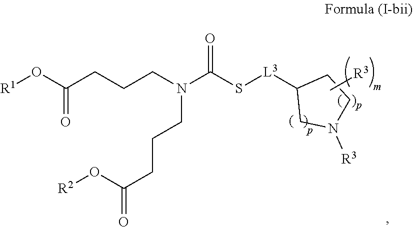
or a pharmaceutically acceptable salt or solvate thereof, wherein:
m is 0, 1, 2, or 3; and
p and q are each independently 0, 1, 2, or 3, wherein q+p is less than or equal to 3.

29. The compound of any of claims 21-28, wherein L³ is a bond.

30. The compound of any of claims 21-28, wherein L³ is —CH₂—.

31. The compound of any of claims 21-30, wherein n is 1.

32. The compound of any of claims 21-30, wherein n is 2.

33. The compound of any of claims 21-30, wherein n is 3.

34. The compound of any of claims 21-33, wherein m is 0 or 1.

35. The compound of any of claims 21-34, wherein R³ is C₁-C₆ alkyl or C₁-C₆ alkenyl, wherein each C₁-C₆ alkyl or C₁-C₆ alkenyl is optionally substituted with 1-3 C₃-C₆ cycloalkyl or —OH.

36. The compound of any of claims 21-35, wherein R³ is C₁-C₃ alkyl.

37. The compound of any of claims 21-36, wherein R³ is —CH₃.

38. The compound of any one of claims 1-20, wherein the compound is a compound of Formula (I-c): 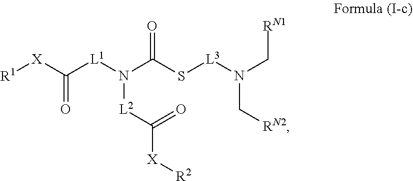
or a pharmaceutically acceptable salt or solvate thereof.

39. The compound of claim 38, wherein X is O.

40. The compound of claim 38, wherein X is NR¹ or NR².

41. The compound of any one of claims 38-40, wherein R^{N1} and R^{N2} are each independently selected from hydrogen, hydroxy-C₁-C₃ alkyl, C₂-C₄ alkenyl, or C₃-C₄ cycloalkyl.

42. The compound of any one of claims 38-41, wherein R^{N1} and R^{N2} are each independently selected from hydrogen, —CH₂CH═CH₂, —CH₂CH₂OH,

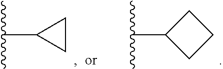

43. The compound of any one of claims 38-42, wherein R^{N1} and R^{N2} are the same.

44. The compound of any one of claims 38-42, wherein R^{N1} and R^{N2} are different.

45. The compound of any one of claims 38-42, wherein one of R^{N1} and R^{N2} is hydrogen and the other one is

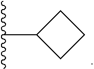 [SMILES: *C1CCC1]

46. A compound, wherein the compound is selected from the group consisting of 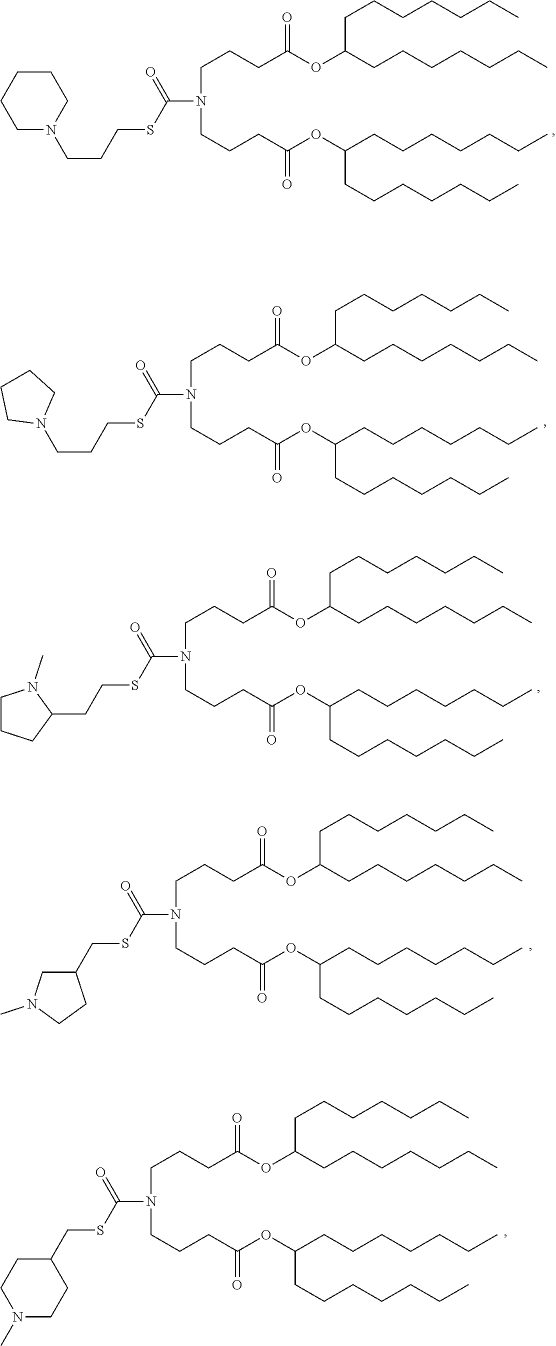 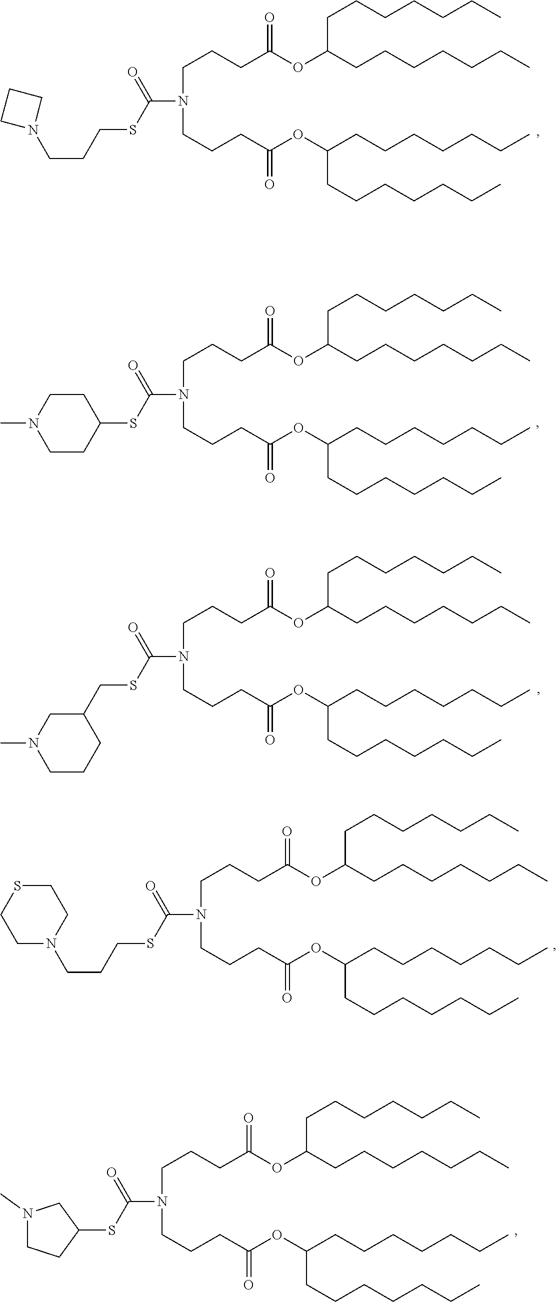 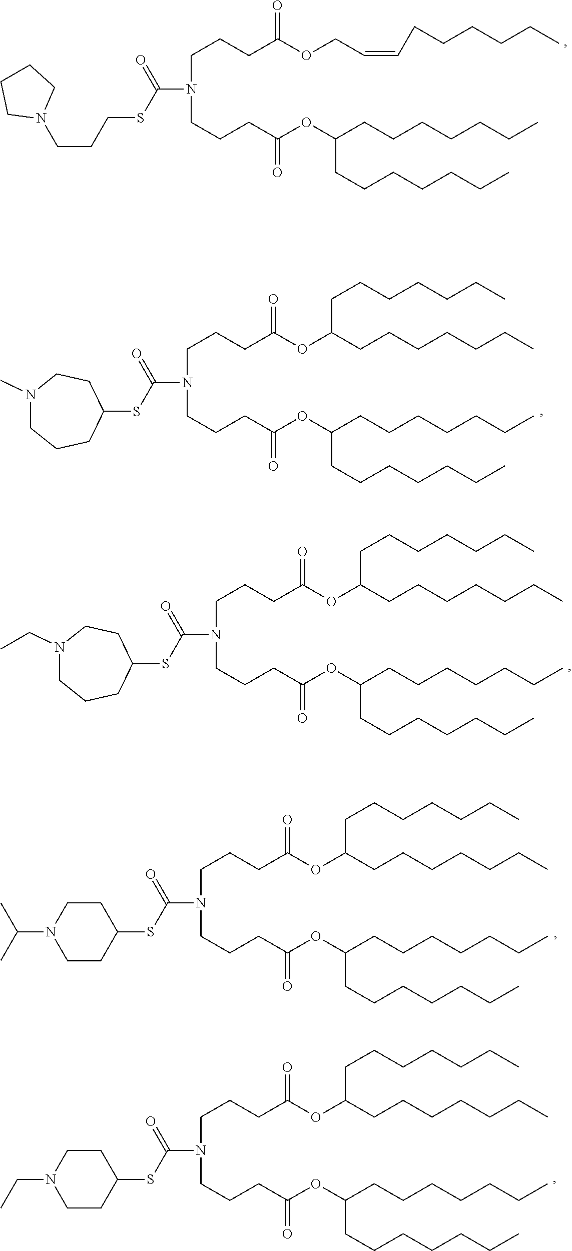 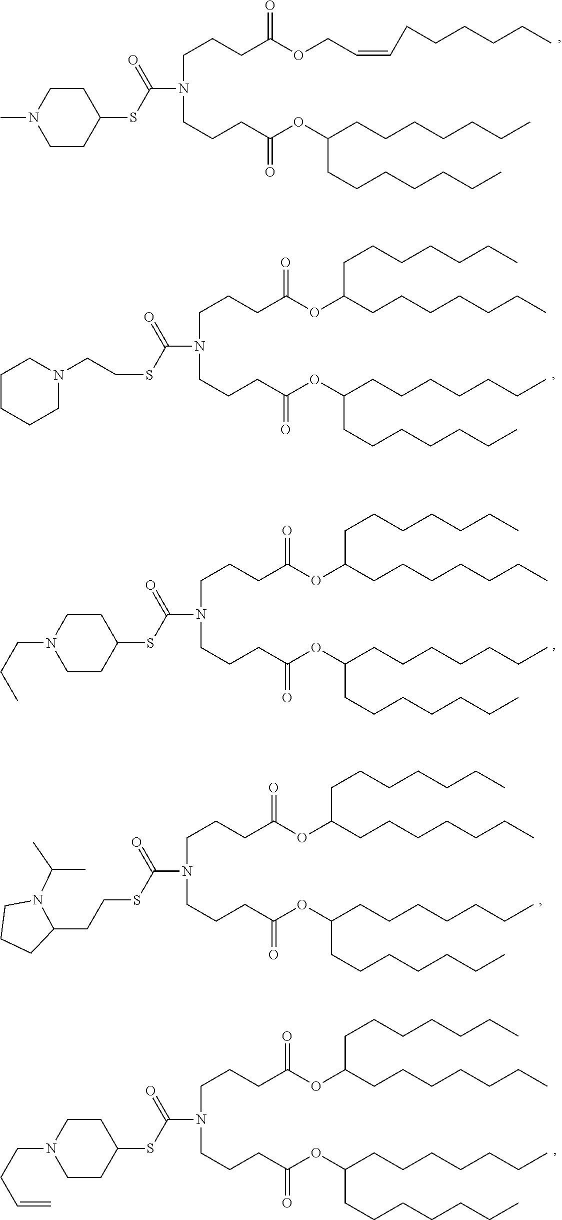 [SMILES: CCCCCC/C=C\COC(=O)CCCN(CCCC(=O)OC(CCCCCCC)CCCCCCC)C(=O)SC1CCN(C)CC1 ; CCCCCCCC(CCCCCCC)OC(=O)CCCN(CCCC(=O)OC(CCCCCCC)CCCCCCC)C(=O)SCCN1CCCCC1 ; CCCCCCCC(CCCCCCC)OC(=O)CCCN(CCCC(=O)OC(CCCCCCC)CCCCCCC)C(=O)SC1CCN(CCC)CC1 ; CCCCCCCC(CCCCCCC)OC(=O)CCCN(CCCC(=O)OC(CCCCCCC)CCCCCCC)C(=O)SCCC1CCCN1C(C)C ; C=CCCN1CCC(SC(=O)N(CCCC(=O)OC(CCCCCCC)CCCCCCC)CCCC(=O)OC(CCCCCCC)CCCCCCC)CC1] 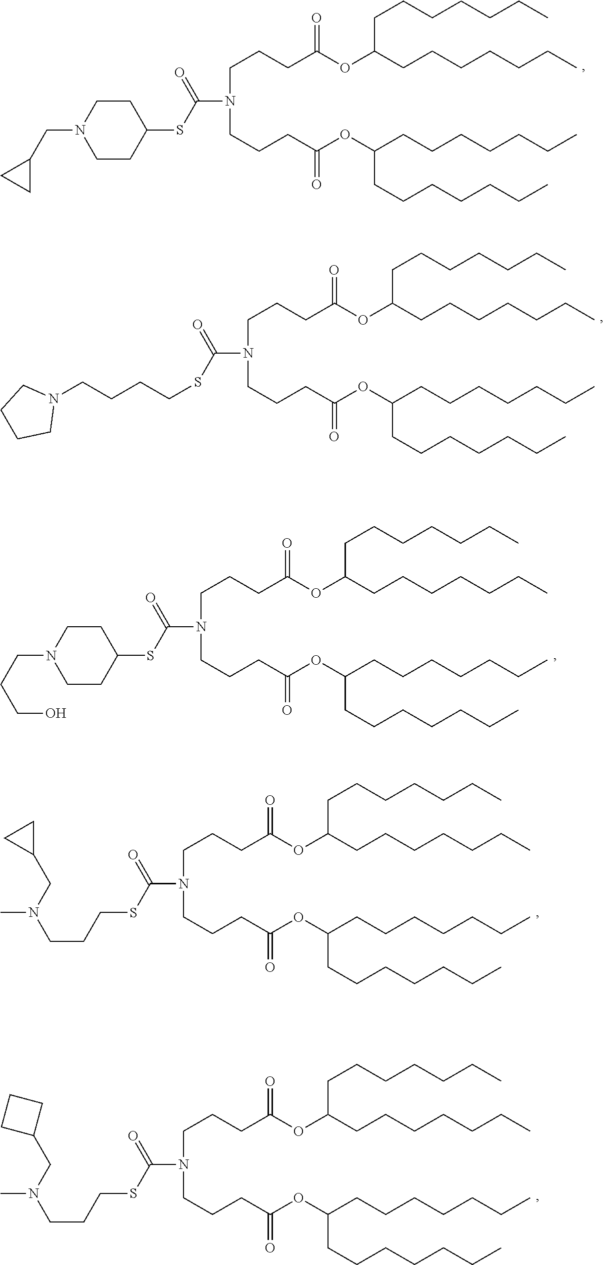 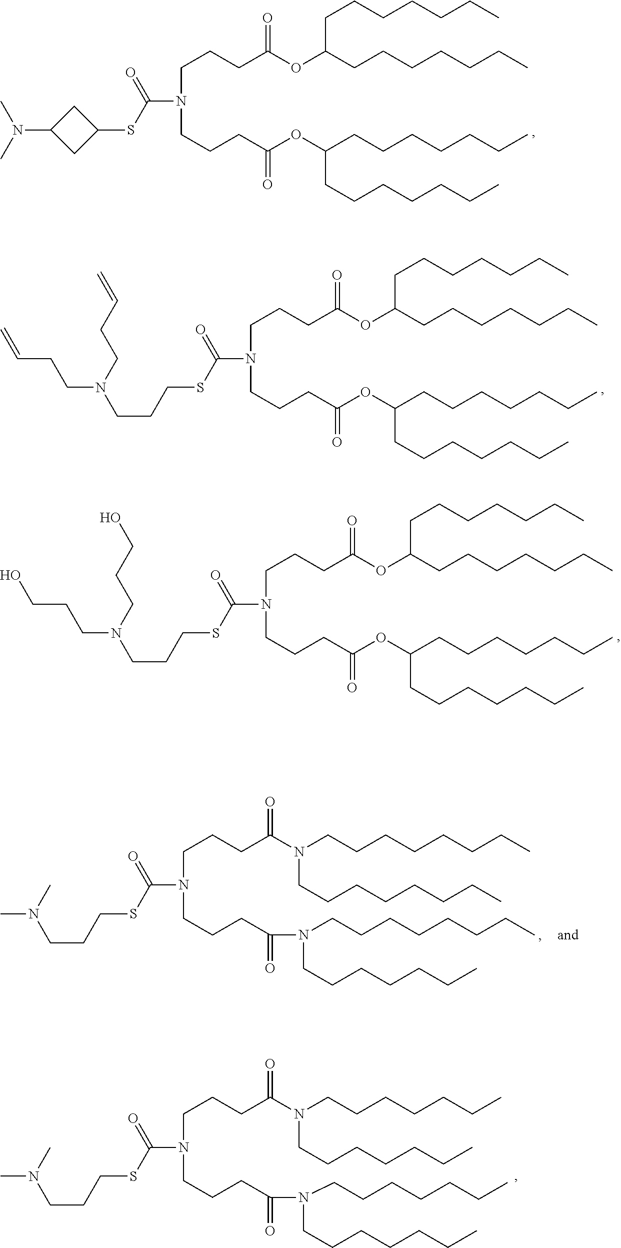
or a pharmaceutically acceptable salt or solvate thereof.

47. The compound of claim 46, wherein the compound is 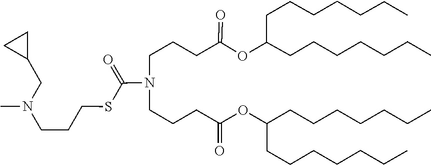 [SMILES: CCCCCCCC(CCCCCCC)OC(=O)CCCN(CCCC(=O)OC(CCCCCCC)CCCCCCC)C(=O)SCCCN(C)CC1CC1]
or a pharmaceutically acceptable salt or solvate thereof.

48. The compound of claim 46, wherein the compound is 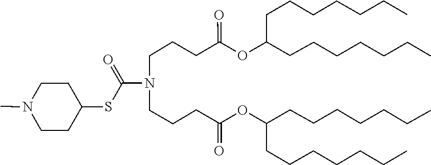 [SMILES: CCCCCCCC(CCCCCCC)OC(=O)CCCN(CCCC(=O)OC(CCCCCCC)CCCCCCC)C(=O)SC1CCN(C)CC1]
or a pharmaceutically acceptable salt or solvate thereof.

49. The compound of claim 46, wherein the compound is 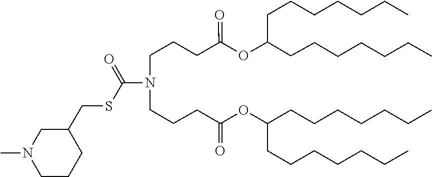 [SMILES: CCCCCCCC(CCCCCCC)OC(=O)CCCN(CCCC(=O)OC(CCCCCCC)CCCCCCC)C(=O)SCC1CCCN(C)C1]
or a pharmaceutically acceptable salt or solvate thereof.

50. A compound, wherein the compound is selected from the group consisting of 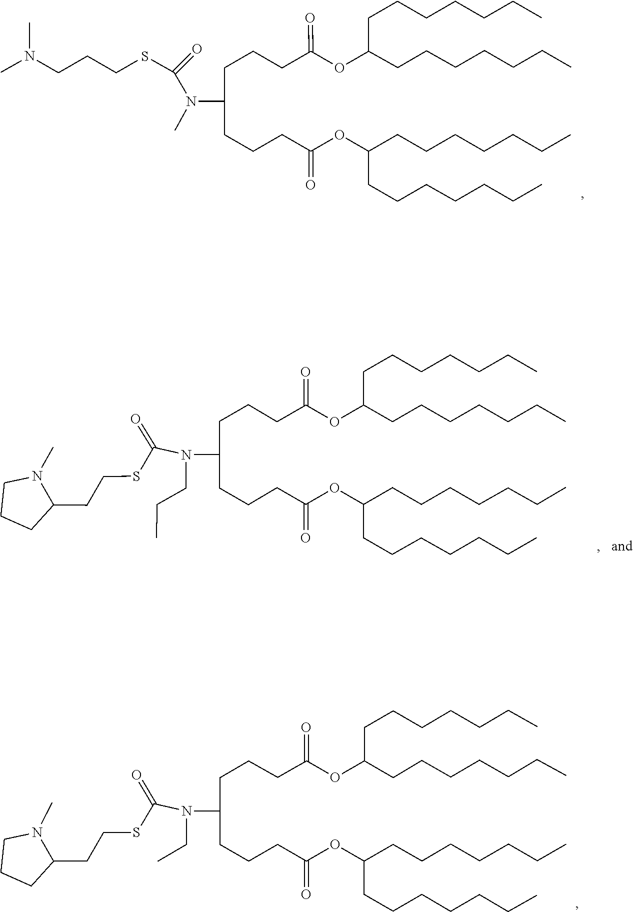 [SMILES: CCCCCCCC(CCCCCCC)OC(=O)CCCC(CCCC(=O)OC(CCCCCCC)CCCCCCC)N(C)C(=O)SCCCN(C)C ; CCCCCCCC(CCCCCCC)OC(=O)CCCC(CCCC(=O)OC(CCCCCCC)CCCCCCC)N(CCC)C(=O)SCCC1CCCN1C ; CCCCCCCC(CCCCCCC)OC(=O)CCCC(CCCC(=O)OC(CCCCCCC)CCCCCCC)N(CC)C(=O)SCCC1CCCN1C]
or a pharmaceutically acceptable salt or solvate thereof.

51. A compound of Formula (A): 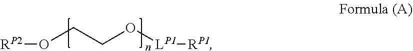
or a pharmaceutically acceptable salt thereof, wherein:
n is an integer between 10 to 200, inclusive of all endpoints;
L^{P1} is —[(CH₂)₀₋₃—C(O)O]₁₋₃—, —(CH₂)₀₋₃—C(O)O—(CH₂)₁₋₃—OC(O)—, or —C(O)N(H)—;
R^{P1} is C₅-C₂₅ alkyl or C₅-C₂₅ alkenyl; and
R^{P2} is hydrogen or —CH₃,
with the proviso that Formula (A) is not HO—(CH₂CH₂O)ₙ—C(O)N(H)—(CH₂)₁₇CH₃.

52. The compound of claim 51, wherein L^{P1} is —CH₂C(O)O—, —CH₂CH₂C(O)O—, —CH₂C(O)OCH₂C(O)O—, —CH₂C(O)OCH₂CH₂OC(O)—, or —C(O)N(H)—.

53. The compound of claim 51 or 52, wherein the compound is a compound of Formula (A-a), Formula (A-b), Formula (A-c), Formula (A-d), or Formula (A-e): 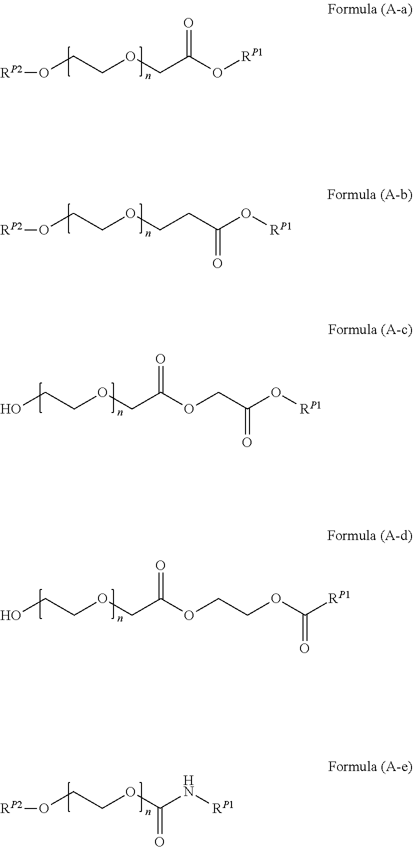
or a pharmaceutically acceptable salt thereof.

54. The compound of any one of claims 51-53, wherein R¹ is C₁₄-Cis alkyl or C₁₄-Cis alkenyl.

55. The compound of any one of claims 51-54, wherein R^{P1} is C₁₄ alkyl, C₁₆ alkyl, or Cis alkyl.

56. The compound of any one of claims 51-55, wherein n is on average about 20, about 40, about 45, about 50, about 68, about 75, or about 100.

57. The compound of any one of claims 46-56, wherein the compound selected from the group consisting of:
HO—(CH₂CH₂O)ₙ—CH₂C(O)O—(CH₂)₁₇CH₃, n is on average about 45;
H₃CO—(CH₂CH₂O)ₙ—CH₂C(O)O—(CH₂)₁₇CH₃, n is on average about 45;
HO—(CH₂CH₂O)ₙ—CH₂C(O)O—(CH₂)₁₅CH₃, n is on average about 45;
HO—(CH₂CH₂O)ₙ—CH₂C(O)O—(CH₂)₁₃CH₃, n is on average about 45; and
HO—(CH₂CH₂O)ₙ—C(O)N(H)—(CH₂)₁₇CH₃, n is on average about 45;
or a pharmaceutically acceptable salt thereof.

58. A lipid nanoparticle (LNP) comprising a compound of any one of claims 1-50.

59. The LNP of claim 58, further comprising a helper lipid, a structural lipid, and a polyethyleneglycol (PEG)-lipid.

60. The LNP of claim 59, wherein the PEG-lipid is a compound of Formula (A′): 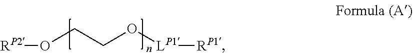
or a pharmaceutically acceptable salt thereof, wherein:
n is an integer between 10 to 200, inclusive of all endpoints;
L^{P1′} is a bond, —C(O)—, —[(CH₂)₀₋₃—C(O)O]₁₋₃—, —(CH₂)₀₋₃—C(O)O—(CH₂)₁₋₃—OC(O)—, or —C(O)N(H)—;
R^{P1′} is C₅-C₂₅ alkyl or C₅-C₂₅ alkenyl; and
R^{P2′} is hydrogen or —CH₃.

61. The LNP of claim 59, wherein the PEG-lipid is a compound of any one of claims 51-57.

62. The LNP of any one of claims 59-61, wherein the PEG-lipid is a compound selected from the group consisting of:
HO—(CH₂CH₂O)ₙ—CH₂C(O)O—(CH₂)₁₇CH₃, n is on average about 45;
H₃CO—(CH₂CH₂O)ₙ—CH₂C(O)O—(CH₂)₁₇CH₃, n is on average about 45;
HO—(CH₂CH₂O)ₙ—CH₂C(O)O—(CH₂)₁₅CH₃, n is on average about 45;
HO—(CH₂CH₂O)ₙ—CH₂C(O)O—(CH₂)₁₃CH₃, n is on average about 45; and
HO—(CH₂CH₂O)ₙ—C(O)N(H)—(CH₂)₁₇CH₃, n is on average about 45;
or a pharmaceutically acceptable salt thereof.

63. The LNP of claim 59 or 60, wherein the PEG-lipid is a compound selected from the group consisting of:
HO—(CH₂CH₂O)ₙ—(CH₂)₁₇CH₃, n is on average about 100;
HO—(CH₂CH₂O)ₙ—(CH₂)₁₇CH₃, n is on average about 20;
HO—(CH₂CH₂O)ₙ—(CH₂)₁₅CH₃, n is on average about 20; and
HO—(CH₂CH₂O)ₙ—C₁₈H₃₅, n is on average about 20;
or a pharmaceutically acceptable salt thereof.

64. The LNP of claim 59 or 60, wherein the PEG-lipid is a compound selected from the group consisting of:
HO—(CH₂CH₂O)ₙ—C(O)—(CH₂)₁₄CH₃, n is on average about 100;
HO—(CH₂CH₂O)ₙ—C(O)—(CH₂)₁₄CH₃, n is on average about 50;
HO—(CH₂CH₂O)ₙ—C(O)—(CH₂)₁₄CH₃, n is on average about 40;
HO—(CH₂CH₂O)ₙ—C(O)—(CH₂)₁₆CH₃, n is on average about 100;
HO—(CH₂CH₂O)ₙ—C(O)—(CH₂)₁₆CH₃, n is on average about 50; and
HO—(CH₂CH₂O)ₙ—C(O)—(CH₂)₁₆CH₃, n is on average about 40;
or a pharmaceutically acceptable salt thereof.

65. The LNP of claim 59, wherein the PEG-lipid is DMG-PEG(2000) or DPG-PEG(2000).

66. A lipid nanoparticle (LNP) comprising a polyethyleneglycol (PEG)-lipid, an ionizable lipid, a helper lipid, and a structural lipid, wherein the LNP has a molar ratio of about 0.001% to about 5% PEG-lipid, and wherein the PEG-lipid is a compound of Formula (A″): 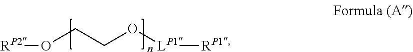
or a pharmaceutically acceptable salt thereof, wherein:
n is an integer between 10 to 200, inclusive of all endpoints;
L^{P1′} is a bond, —[(CH₂)₀₋₃—C(O)O]₁₋₃—, —(CH₂)₀₋₃—C(O)O—(CH₂)₁₋₃—OC(O)—, or —C(O)N(H)—;
R^{1′} is C₅-C₂₅ alkyl or C₅-C₂₅ alkenyl; and
R^{2′} is hydrogen or —CH₃.

67. The LNP of claim 66, wherein L^{P1″} is a bond, —CH₂C(O)O—, —CH₂CH₂C(O)O—, —CH₂C(O)OCH₂C(O)O—, —CH₂C(O)OCH₂CH₂OC(O)—, or —C(O)N(H)—.

68. The LNP of claim 66 or 67, wherein the PEG-lipid is a compound of Formula (A″-a), Formula (A″-b), Formula (A″-c), Formula (A″-cd), Formula (A″-e), or Formula (A″-f): 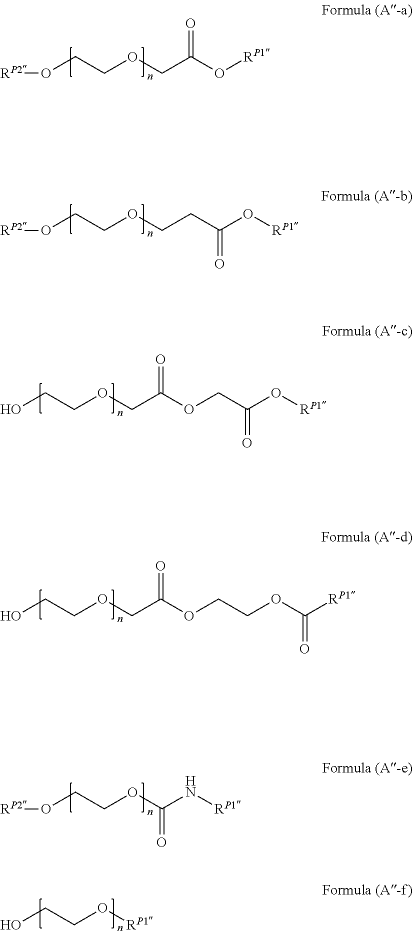
or a pharmaceutically acceptable salt thereof.

69. The LNP of any one of claims 66-68, wherein R^{P1″} is C₁₄-C₁₈ alkyl or C₁₄-C₁₈ alkenyl.

70. The LNP of any one of claims 66-69, wherein R^{P1″} is C₁₄ alkyl, C₁₆ alkyl, or Cis alkyl.

71. The LNP of any one of claims 66-68, wherein the PEG-lipid is a compound of Formula (A″-f1), Formula (A″-f2), or Formula (A″-f3): 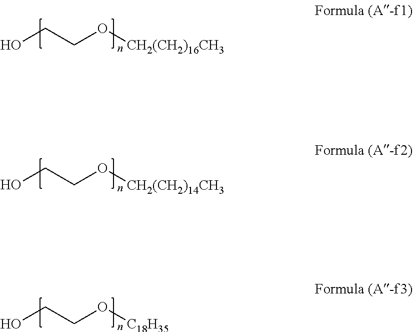
or a pharmaceutically acceptable salt thereof.

72. A lipid nanoparticle (LNP) comprising a polyethyleneglycol (PEG)-lipid, an ionizable lipid, a helper lipid, a structural lipid, and a nucleic acid molecule encoding a viral genome, wherein the LNP has a molar ratio of about 0.001% to about 5% PEG-lipid, and wherein the PEG-lipid is a compound of Formula (B): 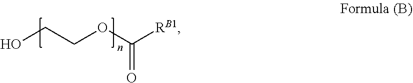
or a pharmaceutically acceptable salt thereof, wherein:
n is an integer between 10 to 200, inclusive of all endpoints; and
R^{B1} IS C₅-C₂₅ alkyl or C₅-C₂₅ alkenyl.

73. The LNP of claim 72, wherein R^{B}1 IS C₁₅-C₁₇ alkyl or C₁₅-C₁₇ alkenyl.

74. The LNP of claim 72 or 73, wherein the PEG-lipid is a compound of Formula (B-a) or Formula (B-b): 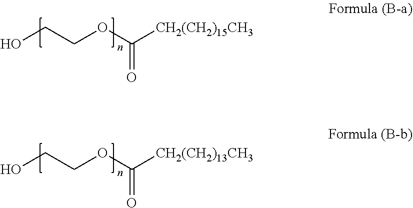
or a pharmaceutically acceptable salt thereof.

75. The LNP of any one of claims 66-74, wherein n is on average about 20, about 40, about 45, about 50, about 68, about 75, or about 100.

76. The LNP of any one of claims 66-75, wherein the PEG-lipid comprises a PEG moiety having an average molecular weight of about 200 daltons to about 10,000 daltons, about 500 daltons to about 7,000 daltons, about 800 daltons to about 6,000 daltons, about 1,000 daltons to about 5,000 daltons, or about 1,500 to about 3,500 daltons.

77. The LNP of any one of claims 66-76, wherein the PEG-lipid comprises a PEG moiety having an average molecular weight of about 800, about 900, about 1,000, about 1,500, about 1,750, about 2,000, about 2,250, about 2,500, about 2,750, about 3,000, about 3,250, about 3,500, about 3,750, about 4,000, about 4,500, or about 5,000 daltons.

78. The LNP of any one of claims 66-77, wherein the PEG-lipid comprises a PEG moiety having an average molecular weight of about 800, about 900, about 1,000 daltons, about 1,500, about 2,000, about 2,500, about 3,000, about 3,500, about 4,000, about 4,500, or about 5,000 daltons.

79. The LNP of any one of claims 66-71 and 75-78, wherein the PEG-lipid is selected from the group consisting of:
HO—(CH₂CH₂O)ₙ—(CH₂)₁₇CH₃, n is on average about 100;
HO—(CH₂CH₂O)ₙ—(CH₂)₁₇CH₃, n is on average about 20;
HO—(CH₂CH₂O)ₙ—(CH₂)₁₅CH₃, n is on average about 20; and
HO—(CH₂CH₂O)ₙ—C₁₈H₃₅, n is on average about 20.

80. The LNP of claim 66-71 and 75-78, wherein the PEG-lipid is a compound selected from the group consisting of:
HO—(CH₂CH₂O)ₙ—CH₂C(O)O—(CH₂)₁₇CH₃, n is on average about 45;
H₃CO—(CH₂CH₂O)ₙ—CH₂C(O)O—(CH₂)₁₇CH₃, n is on average about 45;
HO—(CH₂CH₂O)ₙ—CH₂C(O)O—(CH₂)₁₅CH₃, n is on average about 45;
HO—(CH₂CH₂O)ₙ—CH₂C(O)O—(CH₂)₁₃CH₃, n is on average about 45; and
HO—(CH₂CH₂O)ₙ—C(O)N(H)—(CH₂)₁₇CH₃, n is on average about 45.

81. The LNP of any one of claims 72 to 78, wherein the PEG-lipid is selected from the group consisting of:
HO—(CH₂CH₂O)ₙ—C(O)—(CH₂)₁₄CH₃, n is on average about 100;
HO—(CH₂CH₂O)ₙ—C(O)—(CH₂)₁₄CH₃, n is on average about 50;
HO—(CH₂CH₂O)ₙ—C(O)—(CH₂)₁₄CH₃, n is on average about 40;
HO—(CH₂CH₂O)ₙ—C(O)—(CH₂)₁₆CH₃, n is on average about 100;
HO—(CH₂CH₂O)ₙ—C(O)—(CH₂)₁₆CH₃, n is on average about 50; and
HO—(CH₂CH₂O)ₙ—C(O)—(CH₂)₁₆CH₃, n is on average about 40.

82. The LNP of any one of claims 66-81, wherein the ionizable lipid is selected from DLinDMA, DLin-KC2-DMA, DLin-MC3-DMA (MC3), COATSOME® SS-LC (former name: SS-18/4PE-13), COATSOME® SS-EC (former name: SS-33/4PE-15), COATSOME® SS—OC, COATSOME® SS—OP, Di((Z)-non-2-en-1-yl)₉-((4-dimethylamino)butanoyl)oxy)heptadecanedioate (L-319), N-(2,3-dioleoyloxy)propyl)-N,N,N-trimethylammonium chloride (DOTAP), or a mixture thereof.

83. The LNP of any one of claims 66-81, wherein the ionizable lipid is a compound of Formula (II-1): 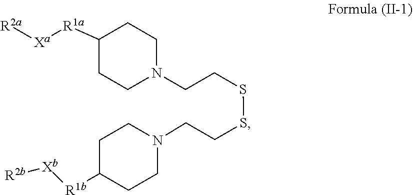
or a pharmaceutically acceptable salt or solvate thereof, wherein:
R^{1a} and R^{1b} are each independently C₁-C₈ aliphatic or —O(C₁-C₈ aliphatic)-, wherein the O atom, when present, is bonded to the piperidine ring;
X^{a} and X^{b} are each independently —C(O)O—*, —OC(O)—*, —C(O)N(Rₓ¹)—*, —N(Rₓ¹)C(O)—*, —O(C═O)N(Rₓ¹)—*, —N(Rₓ¹)(C═O)O—*, or —O—, wherein -* indicates the attachment point to R^{2a} or R^{2b}, respectively, and wherein each occurrence of Rₓ¹ is independently selected from hydrogen and optionally substituted C₁-C₄ alkyl; and
R^{2a} and R^{2b} are each independently a sterol residue, a liposoluble vitamin residue, or an C₁₃-C₂₃ aliphatic.

84. The LNP of any one of claims 66-81, wherein the ionizable lipid is a compound of Formula (II-2): 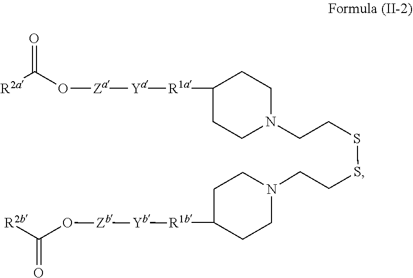
or a pharmaceutically acceptable salt or solvate thereof, wherein:
R^{1a′} and R^{1b′} are each independently C₁-C₈ alkylene or —O(C₁-C₈ alkylene), wherein the O atom, when present, is bonded to the piperidine ring;
Y^{a′} and Y^{b′} are each independently —C(O)O—*, —OC(O)—*, —C(O)N(Rₓ¹)—*, —N(Rₓ¹)C(O)—*, —O(C═O)N(Rₓ¹)—*, —N(Rₓ¹)(C═O)O—*, —N(Rₓ¹)C(O)N(Rₓ¹)—, or —O—, wherein -* indicates the attachment point to R^{2a} or R², and wherein each occurrence of Rₓ¹ is independently selected from hydrogen and optionally substituted C₁-C₄ alkyl;
Z^{a′} and Z^{b′} are each independently optionally substituted arylene-C₀-C₈ alkylene or optionally substituted arylene-C₀-C₈ heteroalkylene, wherein the alkylene or heteroalkylene group is bonded to Y^{a′} and Y^{b′}, respectively;
R^{2a′} and R^{2b′} are each independently a sterol residue, a liposoluble vitamin residue, or an C₁₂-C₂₂ aliphatic.

85. The LNP of claim 83, wherein the ionizable lipid is a compound of Formula (II-1a):

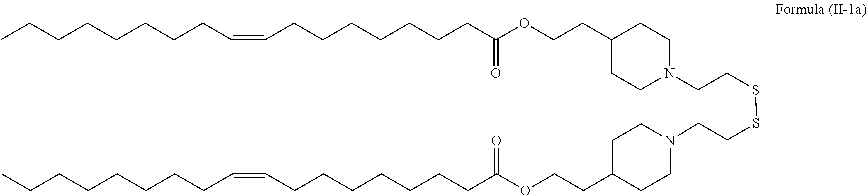 [SMILES: CCCCCCCC/C=C\CCCCCCCC(=O)OCCC1CCN(CCSSCCN2CCC(CCOC(=O)CCCCCCC/C=C\CCCCCCCC)CC2)CC1]

86. The LNP of claim 84, wherein the ionizable lipid is a compound of Formula (II-2a):

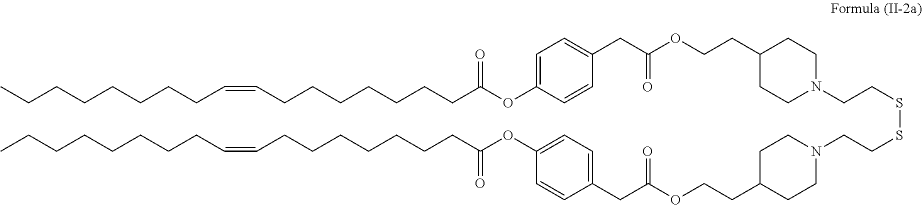 [SMILES: CCCCCCCC/C=C\CCCCCCCC(=O)Oc1ccc(CC(=O)OCCC2CCN(CCSSCCN3CCC(CCOC(=O)Cc4ccc(OC(=O)CCCCCCC/C=C\CCCCCCCC)cc4)CC3)CC2)cc1]

87. The LNP of any one of claims 66-81, wherein the ionizable lipid is a compound of any one of claims 1-50.

88. The LNP of any one of claims 59-87, wherein the helper lipid is selected from distearoyl-sn-glycero-phosphoethanolamine, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoylphosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), monomethyl-phosphatidylethanolamine, dimethylphosphatidylethanolamine, 18-1-trans PE, 1-stearoyl-2-oleoylphosphatidyethanolamine (SOPE), hydrogenated soy phosphatidylcholine (HSPC), egg phosphatidylcholine (EPC), dioleoylphosphatidylserine (DOPS), sphingomyelin (SM), dimyristoyl phosphatidylcholine (DMPC), dimyristoyl phosphatidylglycerol (DMPG), distearoylphosphatidylglycerol (DSPG), dierucoylphosphatidylcholine (DEPC), palmitoyloleyolphosphatidylglycerol (POPG), dielaidoyl-phosphatidylethanolamine (DEPE), lecithin, phosphatidylethanolamine, lysolecithin, lysophosphatidylethanolamine, phosphatidyl serine, phosphatidylinositol, sphingomyelin, egg sphingomyelin (ESM), cephalin, cardiolipin, phosphatidicacid, cerebrosides, dicetylphosphate, lysophosphatidylcholine, dilinoleoylphosphatidylcholine, or a mixture thereof.

89. The LNP of any one of claims 59-88, wherein the helper lipid is DSPC.

90. The LNP of any one of claims 59-89, wherein the structural lipid is a steroid.

91. The LNP of any one of claims 59-90, wherein the structural lipid is cholesterol.

92. The LNP of any one of claims 58-91, wherein the LNP induces a reduced immune response in vivo as compared to a control LNP lacking a PEG-lipid of Formula (A″) or an ionizable lipid of any one of claims 1-50.

93. The LNP of claim 92, wherein the immune response is accelerated blood clearance (ABC) of the LNP.

94. The LNP of claim 92 or 93, wherein the immune response is an IgM response.

95. The LNP of any one of claims 66-71 and 75-94, further comprising a compound of Formula (I), a structural lipid that is cholesterol, a helper lipid that is DSPC, and a PEG-lipid that is a compound of Formula (A″).

96. The LNP of claim 95, wherein the compound of Formula (I) is selected from the group consisting of: 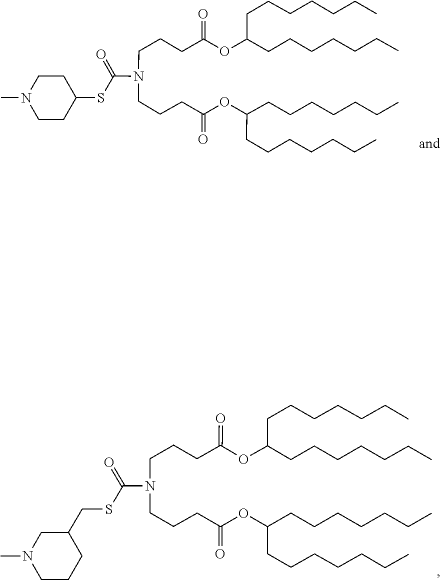
or a pharmaceutically acceptable salt thereof.

97. The LNP of claim 95 or 96, wherein the PEG-lipid is a compound of selected from the group consisting of:
HO—(CH₂CH₂O)ₙ—(CH₂)₁₇CH₃, n is on average about 100;
HO—(CH₂CH₂O)ₙ—CH₂C(O)O—(CH₂)₁₃CH₃, n is on average about 45; and
HO—(CH₂CH₂O)ₙ—CH₂C(O)O—(CH₂)₁₇CH₃, n is on average about 45.

98. The LNP of any one of claims 66-71 and 75-94, comprising a compound of Formula (II-1a), a structural lipid that is cholesterol, a helper lipid that is DSPC, and a PEG-lipid that is a compound of Formula (A″).

99. The LNP of claim 99, wherein the PEG-lipid is selected from the group consisting of:
HO—(CH₂CH₂O)ₙ—CH₂C(O)O—(CH₂)₁₇CH₃, n is on average about 45;
H₃CO—(CH₂CH₂O)ₙ—CH₂C(O)O—(CH₂)₁₇CH₃, n is on average about 45;
HO—(CH₂CH₂O)ₙ—CH₂C(O)O—(CH₂)₁₅CH₃, n is on average about 45;
HO—(CH₂CH₂O)ₙ—CH₂C(O)O—(CH₂)₁₃CH₃, n is on average about 45; and
HO—(CH₂CH₂O)ₙ—C(O)N(H)—(CH₂)₁₇CH₃, n is on average about 45.

100. The LNP of claim 99, wherein the PEG-lipid is HO—(CH₂CH₂O)ₙ—(CH₂)₁₇CH₃, n is on average about 100.

101. The LNP of any one of claims 72-94, comprising a compound of Formula (II-1a), a structural lipid that is cholesterol, a helper lipid that is DSPC, and a PEG-lipid that is a compound of Formula (B).

102. The LNP of claim 101, wherein the PEG-lipid is selected from the group consisting of:
HO—(CH₂CH₂O)ₙ—C(O)—(CH₂)₁₆CH₃, n is on average about 100;
HO—(CH₂CH₂O)ₙ—C(O)—(CH₂)₁₆CH₃, n is on average about 50; and
HO—(CH₂CH₂O)ₙ—C(O)—(CH₂)₁₆CH₃, n is on average about 40.

103. The LNP of any of claims 58-81 and 88-97, wherein the LNP comprises a molar ratio of about 40% O about 70%, such as about 45% to about 55%, or about 49% to about 64% of a compound of any one of claims 1-50.

104. The LNP of any of claims 58-81, 88-97, and 103, wherein the LNP comprises a molar ratio of about 40%, about 45%, about 50 %, about 55%, about 58%, or about 60% of a compound of any of claims 1-50.

105. The LNP of any one of claims 58-104, wherein the LNP comprises a molar ratio of about 40% to about 70%. such as about 45% to about 55%, or about 49% to about 64% ionizable lipid.

106. The LNP of any one of claims 58-105, wherein the LNP comprises a molar ratio of about 40%, about 45%, about 50W % about 55%, about 58%, or about 60% ionizable lipid.

107. The LNP of any one of claims 58-106, wherein the LNP comprises a molar ratio of about 0.10% to about 4%, such as about 0.2% to about 0.8 mol %, about 0.4% to about 0.6 mol %, about 0.7% to about 1.3%, about 1.2% to about 1.8%, or about 1% to about 3.5 mol % PEG-lipid.

108. The LNP of any one of claims 58-107, wherein the LNP comprises a molar ratio of about 0.25%, about 0.5%, about 1.5%, or about 3% PEG-lipid.

109. The LNP of any one of claims 58-108, wherein the LNP comprises a molar ratio of about 5% to about 50%, such as about 5% to about 10%, about 25% to about 35%, or about 35% to about 50% structural lipid.

110. The LNP of any one of claims 58-109, wherein the LNP comprises a molar ratio of about 20%, about 22.5%, about 25%, about 27.5%, about 30%, about 32.5%, about 35%, about 37.5%, about 40%, about 42.5%, about 45%, or about 50% structural lipid.

111. The LNP of any one of claims 58-110, wherein the LNP comprises a molar ratio of about 5% to about 50%, such as about 5% to about 10%, about 10% to about 25%, or about 25% to about 50% helper lipid.

112. The LNP of any one of claims 58-111, wherein the LNP comprises a molar ratio of about 5%, about 7%, about 9%, about 12%, about 15%, about 20%, about 25%, or about 30% helper lipid.

113. The LNP of any one of claims 58-112, wherein the LNP comprises a molar ratio of about 45% to about 55% of ionizable lipid, about 5% to about 9% helper lipid, about 36% to about 44% structural lipid, and about 2.5% to about 3.5% PEG-lipid.

114. The LNP of claim 113, wherein the LNP comprises a molar ratio of about 45% to about 55% of a compound of any one of claims 1-50, about 5% to about 9% DSPC, about 36% to about 44% cholesterol, and about 2.5% to about 3.5% DMG-PEG(2000).

115. The LNP of any one of claims 58-112, wherein the LNP comprises a molar ratio of about 49% to about 60% of ionizable lipid, about 18% to about 22% helper lipid, about 22% to about 28% structural lipid, and about 0.2% to about 0.8% PEG-lipid.

116. The LNP of any one of claim 115, wherein the LNP comprises a molar ratio of about 49% to about 60% of a compound of any one of claims 1-50, about 18% to about 22% helper lipid, about 22% to about 28% structural lipid, and about 0.2% to about 0.8% PEG-lipid, wherein the PEG-lipid is selected from the group consisting of:
HO—(CH₂CH₂O)ₙ—CH₂C(O)O—(CH₂)₁₇CH₃, n is on average about 45;
H₃CO—(CH₂CH₂O)ₙ—CH₂C(O)O—(CH₂)₁₇CH₃, n is on average about 45;
HO—(CH₂CH₂O)ₙ—CH₂C(O)O—(CH₂)₁₅CH₃, n is on average about 45;
HO—(CH₂CH₂O)ₙ—CH₂C(O)O—(CH₂)₁₃CH₃, n is on average about 45; and
HO—(CH₂CH₂O)ₙ—C(O)N(H)—(CH₂)₁₇CH₃, n is on average about 45.

117. The LNP of any one of claims 58-112, wherein the LNP comprises a molar ratio of about 44% to about 54% ionizable lipid, about 19% to about 25% helper lipid, about 25% to about 33% structural lipid, and about 0.2% to about 0.8% PEG-lipid.

118. The LNP of claim 117, wherein the LNP comprises molar ratio of about 44% to about 54% compound of Formula (II-1a), about 19% to about 25% DSPC, about 25% to about 33% cholesterol, and about 0.2% to about 0.8% PEG-lipid, wherein the PEG-lipid is selected from the group consisting of:
HO—(CH₂CH₂O)ₙ—(CH₂)₁₇CH₃, n is on average about 100;
HO—(CH₂CH₂O)ₙ—(CH₂)₁₇CH₃, n is on average about 20;
HO—(CH₂CH₂O)ₙ—(CH₂)₁₅CH₃, n is on average about 20;
HO—(CH₂CH₂O)ₙ—C₁₈H₃₅, n is on average about 20;
HO—(CH₂CH₂O)ₙ—C(O)—(CH₂)₁₄CH₃, n is on average about 100;
HO—(CH₂CH₂O)ₙ—C(O)—(CH₂)₁₄CH₃, n is on average about 50;
HO—(CH₂CH₂O)ₙ—C(O)—(CH₂)₁₄CH₃, n is on average about 40;
HO—(CH₂CH₂O)ₙ—C(O)—(CH₂)₁₆CH₃, n is on average about 100;
HO—(CH₂CH₂O)ₙ—C(O)—(CH₂)₁₆CH₃, n is on average about 50; and
HO—(CH₂CH₂O)ₙ—C(O)—(CH₂)₁₆CH₃, n is on average about 40.

119. The LNP of any one of claims 58-112, wherein the LNP comprises a molar ratio of about 44% to about 54% ionizable lipid, about 19% to about 25% helper lipid, about 24% to about 32% structural lipid, and about 1.2% to about 1.8% PEG-lipid.

120. The LNP of claim 119, wherein the LNP comprises a molar ratio of about 44% to about 54% compound of Formula (II-1a), about 19% to about 25% DSPC, about 24% to about 32% cholesterol, and about 1.2% to about 1.8% PEG-lipid, wherein the PEG-lipid is selected from the group consisting of:
HO—(CH₂CH₂O)ₙ—(CH₂)₁₇CH₃, n is on average about 100;
HO—(CH₂CH₂O)ₙ—(CH₂)₁₇CH₃, n is on average about 20;
HO—(CH₂CH₂O)ₙ—(CH₂)₁₅CH₃, n is on average about 20;
HO—(CH₂CH₂O)ₙ—C₁₈H₃₅, n is on average about 20;
HO—(CH₂CH₂O)ₙ—C(O)—(CH₂)₁₄CH₃, n is on average about 100;
HO—(CH₂CH₂O)ₙ—C(O)—(CH₂)₁₄CH₃, n is on average about 50;
HO—(CH₂CH₂O)ₙ—C(O)—(CH₂)₁₄CH₃, n is on average about 40;
HO—(CH₂CH₂O)ₙ—C(O)—(CH₂)₁₆CH₃, n is on average about 100;
HO—(CH₂CH₂O)ₙ—C(O)—(CH₂)₁₆CH₃, n is on average about 50; and
HO—(CH₂CH₂O)ₙ—C(O)—(CH₂)₁₆CH₃, n is on average about 40.

121. The LNP of any one of claims 58-112, wherein the LNP comprises a molar ratio of about 44% to about 54% ionizable lipid, about 8% to about 14% helper lipid, about 35% to about 43% structural lipid, and about 1.2% to about 1.8% PEG-lipid.

122. The LNP of claim 121 wherein the LNP comprises a molar ratio of about 44% to about 54% compound of Formula (II-1a), about 8% to about 14% DSPC, about 35% to about 43% cholesterol, and about 1.2% to about 18% PEG-lipid, wherein the PEG-lipid is selected from the group consisting of:
HO—(CH₂CH₂O)ₙ—(CH₂)₁₇CH₃, n is on average about 100;
HO—(CH₂CH₂O)ₙ—(CH₂)₁₇CH₃, n is on average about 20;
HO—(CH₂CH₂O)ₙ—(CH₂)₁₅CH₃, n is on average about 20;
HO—(CH₂CH₂O)ₙ—C₁₈H₃₅, n is on average about 20;
HO—(CH₂CH₂O)ₙ—C(O)—(CH₂)₁₄CH₃, n is on average about 100;
HO—(CH₂CH₂O)ₙ—C(O)—(CH₂)₁₄CH₃, n is on average about 50;
HO—(CH₂CH₂O)ₙ—C(O)—(CH₂)₁₄CH₃, n is on average about 40;
HO—(CH₂CH₂O)ₙ—C(O)—(CH₂)₁₆CH₃, n is on average about 100;
HO—(CH₂CH₂O)ₙ—C(O)—(CH₂)₁₆CH₃, n is on average about 50; and
HO—(CH₂CH₂O)ₙ—C(O)—(CH₂)₁₆CH₃, n is on average about 40.

123. The LNP of any one of claims 58-71 and 75-122, wherein the lipid nanoparticle encapsulates a payload molecule.

124. The LNP of claim 123, wherein the payload molecule comprises one or more of nucleic acids, anionic proteins, anionic peptides, or a combination thereof.

125. The LNP of claim 124, wherein the payload molecule comprises a nucleic acid molecule.

126. The LNP of claim 125, wherein the nucleic acid molecule comprises a single-stranded RNA (ssRNA), an siRNA, a microRNA, an mRNA, a circular RNA, a small activating RNA, a guide RNA for CRISPR, a self-amplifying RNA, a viral RNA (vRNA), a single-stranded DNA (ssDNA), a double-stranded DNA (dsDNA), a complementary DNA (cDNA), a closed circular DNA (ccDNA), a replicon, or a combination thereof.

127. The LNP of claim 125 or 126, wherein the nucleic acid molecule comprises a nucleotide sequence encoding one or more therapeutic proteins.

128. The LNP of claim 127, wherein the therapeutic protein is a cytokine (e.g., erythropoietin), a coagulation factor, an antibody, a bispecific T cell engager, or a combination thereof.

129. The LNP of any one of claims 125-128, wherein the nucleic acid molecule comprises a nucleotide sequence derived from a viral genome.

130. The LNP of claim 129, wherein the viral genome is a positive single-stranded RNA viral genome a positive single-stranded RNA viral genome.

131. The LNP of claim 129 wherein the viral genome encodes an oncolytic virus (e.g., Coxsackievirus A21 (CVA21), Seneca Valley virus (SVV), Togaviridae, or Alphavirus (e.g., Sindbis virus, Semliki Forest virus, Ross River virus, or Chikungunya virus)).

132. The LNP of claim 124, wherein the payload molecule comprises a synthetic RNA viral genome encoding a coxsackievirus, and optionally wherein the coxsackievirus is a CVA21 strain.

133. The LNP of claim 124, wherein the payload molecule comprises a synthetic RNA viral genome encoding an SVV.

134. The LNP of claim 132 or 133, wherein the payload molecule further encodes an exogenous protein, wherein the exogenous protein is a fluorescent protein, an enzymatic protein, a cytokine, a chemokine, an antigen-binding molecule capable of binding to a cell surface receptor, or a ligand for a cell-surface receptor.

135. The LNP of any one of claims 72-122, wherein the viral genome is a positive single-stranded RNA viral genome.

136. The LNP of claim 135, wherein the viral genome encodes an oncolytic virus (e.g., Coxsackievirus A21 (CVA21) or Seneca Valley virus (SVV), Togaviridae, or Alphavirus (e.g., Sindbis virus, Semliki Forest virus, Ross River virus, or Chikungunya virus)).

137. The LNP of claim 135, wherein the viral genome is a synthetic RNA viral genome encoding a coxsackievirus, and optionally wherein the coxsackievirus is a CVA21 strain.

138. The LNP of claim 135, wherein the viral genome is a synthetic RNA viral genome encoding an SVV.

139. The LNP of any one of claims 72-122 and 135-138, wherein the viral genome further comprises an exogenous protein, wherein the exogenous protein is a fluorescent protein, an enzymatic protein, a cytokine, a chemokine, an antigen-binding molecule capable of binding to a cell surface receptor, or a ligand for a cell-surface receptor.

140. The LNP of any one of claims 72-122 and 125-139, wherein the LNP has a lipid-nitrogen-to-phosphate (N:P) ratio of about 1 to about 25.

141. The LNP of any one of claims 72-122 and 125-140, wherein the LNP has a N:P ratio of about 14.

142. The LNP of any one of claims 72-122 and 125-140, wherein the LNP has a N:P ratio of about 9.

143. A pharmaceutical composition comprising a compound of any of claims 1-57 or a LNP of any of claims 58-142 and pharmaceutically acceptable excipient, carrier or diluent.

144. A pharmaceutical composition comprising: (1) a payload molecule; and (2) a LNP of any one of claims 66-71 and 75-142.

145. The pharmaceutical composition of claim 143 or 144, wherein the pharmaceutical composition has a half-life in vivo comparable to that of a pre-determined threshold value.

146. The pharmaceutical composition of claim 143 or 144, wherein the pharmaceutical composition has a half-life in vivo greater than that of a pre-determined threshold value.

147. The pharmaceutical composition of claim 143 or 144, wherein the pharmaceutical composition has a half-life in vivo shorter than that of a pre-determined threshold value.

148. The pharmaceutical composition of claim 143 or 144, wherein the pharmaceutical composition has an AUC in vivo comparable to that of a pre-determined threshold value.

149. The pharmaceutical composition of claim 143 or 144, wherein the pharmaceutical composition has an AUC in vivo greater than that of a pre-determined threshold value.

150. The pharmaceutical composition of claim 143 or 144, wherein the pharmaceutical composition has an AUC in vivo less than that of a pre-determined threshold value.

151. The pharmaceutical composition of any one of claims 145-150, wherein the pre-determined threshold value is determined in a control composition comprising the same payload molecule and LNP except that the LNP lacks a PEG-lipid of Formula (A′) or an ionizable lipid of any one of claims 1-50.

152. The pharmaceutical composition of any one of claims 143-151, wherein the LNP has an average diameter of about 50 nm, about 60 nm, about 70 nm, about 80 nm, about 90 nm, about 100 nm, about 110 nm, about 120 nm, or about 125 nm.

153. The pharmaceutical composition of any one of claims 143-152, wherein the encapsulation efficiency of the payload molecule by the LNP is about 70%, about 75%, about 80%, about 85%, about 90%, about 91%, about 92%, about 93%, about 94%, about 95%, about 96%, about 97%, about 98%, about 99%, or 100%.

154. The pharmaceutical composition of any one of claims 143-153, wherein the pharmaceutical composition has a total lipid concentration of about 10 mM, about 20 mM, about 30 mM, about 40 mM, or about 50 mM.

155. The pharmaceutical composition of any one of claims 143-154, wherein the pharmaceutical composition is formulated at a pH of about 2.5, about 3, about 3.5, about 4, about 4.5, about 5, about 5.5, or about 6.

156. The pharmaceutical composition of any one of claims 143-155, wherein the pharmaceutical composition is formulated for multiple administrations.

157. The pharmaceutical composition of claim 156, wherein a subsequent administration is administered at least 3 days, at least 5 days, at least 7 days, at least 9 days, at least 11 days, at least 14 days, or at least 21 days after a first administration.

158. The pharmaceutical composition of any one of claims 144-157, wherein the payload molecule comprises a nucleic acid molecule.

159. The pharmaceutical composition of any one of claims 144-158, wherein the payload molecule comprises a synthetic RNA viral genome encoding a Coxsackievirus or an SVV.

160. The pharmaceutical composition of any one of claims 144-157, wherein the viral genome comprised in the LNP is a synthetic RNA viral genome encoding a Coxsackievirus or an SVV.

161. The pharmaceutical composition of any one of claims 144-160, further comprising a pharmaceutically acceptable carrier.

162. A method of treating a disease or disorder, comprising administering to a patient in need thereof a lipid nanoparticle of any of claims 58-142 or a pharmaceutical composition of any one of claims 143-161.

163. The method of claim 162, wherein the disease or disorder is cancer.

164. The method of claim 163, wherein the cancer is selected from the group consisting of lung cancer, breast cancer, ovarian cancer, cervical cancer, prostate cancer, testicular cancer, colorectal cancer, colon cancer, pancreatic cancer, liver cancer, renal cell carcinoma, gastric cancer, head and neck cancer, thyroid cancer, malignant glioma, glioblastoma, melanoma, B-cell chronic lymphocytic leukemia, multiple myeloma, monoclonal gammopathy of undetermined significance (MGUS), Merkel cell carcinoma, diffuse large B-cell lymphoma (DLBCL), sarcoma, a neuroblastoma, a neuroendocrine cancer, a rhabdomyosarcoma, a medulloblastoma, a bladder cancer, and marginal zone lymphoma (MZL).

165. The method of claim 163, wherein the cancer is selected from the groups consisting of lung cancer, breast cancer, colon cancer, pancreatic cancer, bladder cancer, renal cell carcinoma, ovarian cancer, gastric cancer, and liver cancer.

166. The method of claim 163, wherein the cancer is renal cell carcinoma, lung cancer, or liver cancer.

167. The method of any one of claims 164-166, wherein the lung cancer is small cell lung cancer or non-small cell lung cancer (e.g., squamous cell lung cancer or lung adenocarcinoma).

168. The method of any one of claims 164-166, wherein the liver cancer is hepatocellular carcinoma (HCC) (e.g., Hepatitis B virus associated HCC).

169. The method of claim 164, wherein the prostate cancer is treatment-emergent neuroendocrine prostate cancer.

170. The method of claim 163, wherein the cancer is lung cancer, liver cancer, prostate cancer (e.g., CRPC-NE), bladder cancer, pancreatic cancer, colon cancer, gastric cancer, breast cancer, neuroblastoma, renal cell carcinoma, ovarian cancer, rhabdomyosarcoma, medulloblastoma, neuroendocrine cancer, Merkel cell carcinoma, or melanoma.

171. The method of claim 163, wherein the cancer is small cell lung cancer (SCLC) or neuroblastoma.

172. The method of any one of claims 163-171, wherein the administration of the pharmaceutical composition delivers a payload into tumor cells.

173. The method of any one of claims 163-172, wherein the administration of the pharmaceutical composition inhibits the tumor growth.

174. The method of any one of claims 162-173, wherein the LNP or pharmaceutical composition is administered parenterally.

175. The method of any one of claims 162-174, wherein the LNP or pharmaceutical composition is administered is administered intratumorally and/or intravenously.
